(19) United States
(12) Patent Application Publication
(10) Pub. No.: US 2024/0357926 A1
(43) Pub. Date: Oct. 24, 2024

(54) MATERIALS FOR ORGANIC ELECTROLUMINESCENT DEVICES

(71) Applicant: Merck Patent GmbH, Darmstadt (DE)

(72) Inventors: Amir PARHAM, Frankfurt am Main (DE); Jonas KROEBER, Frankfurt am Main (DE); Christian EHRENEICH, Darmstadt (DE); Jonas ENGELHART, Darmstadt (DE); Christian EICKHOFF, Mannheim (DE); Jens PFALZGRAF, Otzberg (DE)

(21) Appl. No.: 17/414,758

(22) PCT Filed: Dec. 17, 2019
(86) PCT No.: PCT/EP2019/085506

§ 371 Date: Jun. 16, 2021

(30) Foreign Application Priority Data
Dec. 19, 2018 (EP) 18213861.0

(51) Int. Cl. H10K 85/60; C07D 471/06; C07D 519/00; C09K 11/06; H10K 50/12

(52) U.S. Cl. CPC H10K 85/6572; C07D 471/06; C07D 519/00; C09K 11/06; H10K 85/615; H10K 85/657; H10K 85/6574; H10K 85/6576; C09K 2211/1018; H10K 50/12

(57) ABSTRACT

The present invention relates to compounds suitable for use in electronic devices, and to electronic devices, especially organic electroluminescent devices, comprising these compounds.

The present invention relates to materials for use in electronic devices, especially in organic electroluminescent devices, and to electronic devices, especially organic electroluminescent devices comprising these materials.

Emitting materials used in organic electroluminescent devices (OLEDs) are frequently phosphorescent organometallic complexes. In general terms, there is still a need for improvement in OLEDs, especially also in OLEDs which exhibit triplet emission (phosphorescence), for example with regard to efficiency, operating voltage and lifetime. The properties of phosphorescent OLEDs are not just determined by the triplet emitters used. More particularly, the other materials used, such as matrix materials, are also of particular significance here. Improvements to these materials can thus also lead to improvements in the OLED properties.

The problem addressed by the present invention is that of providing compounds that are suitable for use in an OLED, especially as matrix material for phosphorescent emitters, but also as electron transport materials, hole blocker materials or exciton blocker materials, and which lead to an improved lifetime therein.

It has been found that, surprisingly, particular compounds described in detail hereinafter solve this problem and are of good suitability for use in OLEDs. These OLEDs especially have a distinctly improved lifetime, high efficiency and low operating voltage. The present invention therefore provides these compounds and electronic devices, especially organic electroluminescent devices, comprising such compounds.

WO 2011/137951 discloses the use of lactams as matrix materials for phosphorescent emitters. There is no disclosure of lactam derivatives having a substitution pattern as described below.

The present invention provides a compound of formula (1)

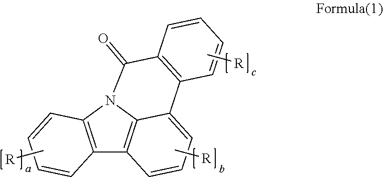

with the proviso that at least one R radical that represents a Y group is present, and where the symbols and indices used are as follows:
Y is the same or different at each instance and is a group of the following formula (2):

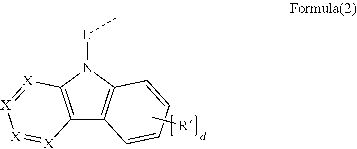

where the dotted bond indicates the linkage of this group in the formula (1);
X is the same or different at each instance and is CR′ or two adjacent X are a group of the following formula (3), and the remaining X are the same or different at each instance and are CR′:

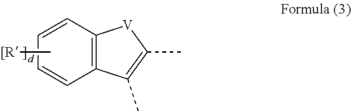

where the dotted bonds indicate the linkage of this group in the formula (2);
V is NR′, C(R′)₂, O or S;
L is an aromatic or heteroaromatic ring system which has 5 to 30 aromatic ring atoms and may be substituted by one or more R′ radicals;
R, R′ is the same or different at each instance and is H, D, F, Cl, Br, I, N(Ar′)₂, N(R¹)₂, OAr′, SAr′, CN, NO₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by Si(R¹)₂, C═O, NR¹, O, S or CONR¹, or an aromatic or heteroaromatic ring system which has 5 to 60 aromatic ring atoms, preferably 5 to 40 aromatic ring atoms, and may be substituted in each case by one or more R¹ radicals; at the same time, two R radicals together may also form an aliphatic, heteroaliphatic, aromatic or heteroaromatic ring system; in addition, two R′ radicals together may also form an aliphatic, heteroaliphatic, aromatic or heteroaromatic ring system; with the proviso that at least one R radical is a Y group;
Ar′ is the same or different at each instance and is an aromatic or heteroaromatic ring system which has 5 to 40 aromatic ring atoms and may be substituted by one or more R¹ radicals;
R¹ is the same or different at each instance and is H, D, F, C, Br, I, N(R²)₂, CN, NO₂, OR², SR², Si(R²)₃, B(OR²)₂, COOR², C(═O)R², P(═O)(R²)₂, S(═O)R², S(═O)₂R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may each be substituted by one or more R² radicals, where one or more nonadjacent CH₂ groups may be replaced by Si(R²)₂, C═O, NR², O, S or CONR² and where one or more hydrogen atoms in the alkyl, alkenyl or alkynyl group may be replaced by D, F, Cl, Br, I or CN, or an aromatic or heteroaromatic ring system which has 5 to 40 aromatic ring atoms and may be substituted in each case by one or more R² radicals; at the same time, two or more R¹ radicals together may form an aliphatic, heteroaliphatic, aromatic or heteroaromatic ring system;
R² is the same or different at each instance and is H, D, F, CN or an aliphatic, aromatic or heteroaromatic organic radical, especially a hydrocarbyl radical, having 1 to 20 carbon atoms, in which one or more hydrogen atoms may also be replaced by F;
a is 0, 1, 2, 3 or 4;
b is the same or different at each instance and is 0, 1, 2 or 3;
c is 0, 1, 2, 3 or 4;
d is the same or different at each instance and is 0, 1, 2, 3 or 4;
with the proviso that a+b+c≥1;
with exclusion of the following compound from the invention:

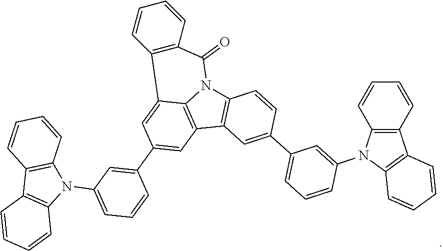 [SMILES: O=c1c2ccccc2c2cc(-c3cccc(-n4c5ccccc5c5ccccc54)c3)cc3c4cc(-c5cccc(-n6c7ccccc7c7ccccc76)c5)ccc4n1c23]

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatic systems joined to one another by a single bond, for example biphenyl, by contrast, are not referred to as an aryl or heteroaryl group but as an aromatic ring system.

An aromatic ring system in the context of this invention contains 6 to 60 carbon atoms, preferably 6 to 40 carbon atoms, in the ring system. A heteroaromatic ring system in the context of this invention contains 2 to 60 carbon atoms, preferably 2 to 40 carbon atoms, and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a nonaromatic unit, for example a carbon, nitrogen or oxygen atom. These shall likewise be understood to mean systems in which two or more aryl or heteroaryl groups are joined directly to one another, for example biphenyl, terphenyl, bipyridine or phenylpyridine. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group. Preferred aromatic or heteroaromatic ring systems are simple aryl or heteroaryl groups and groups in which two or more aryl or heteroaryl groups are joined directly to one another, for example biphenyl or bipyridine, and also fluorene or spirobifluorene.

In the context of the present invention, an aliphatic hydrocarbyl radical or an alkyl group or an alkenyl or alkynyl group which may contain 1 to 40 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group OR¹ having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group SR¹ having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, more preferably F or CN.

An aromatic or heteroaromatic ring system which has 5-60 aromatic ring atoms and may also be substituted in each case by the abovementioned R² radicals or a hydrocarbyl radical and which may be joined to the aromatic or heteroaromatic system via any desired positions is especially understood to mean groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived by combination of these systems.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

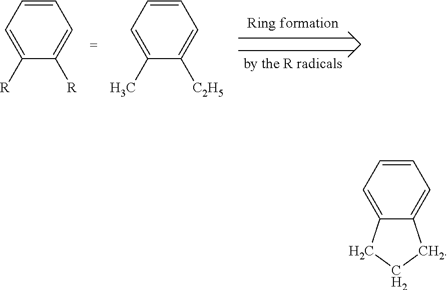

In addition, however, the abovementioned wording should also be understood to mean that, if one of the two radicals is hydrogen, the second radical binds to the position to which the hydrogen atom was bonded, forming a ring. This shall be illustrated by the following scheme:

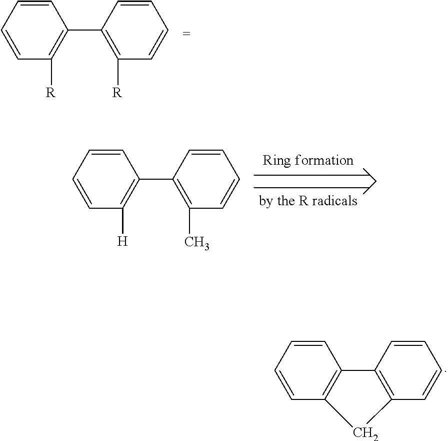

Various isomers arise according to the position of the Y group. These are represented hereinafter by the formulae (4) to (6)

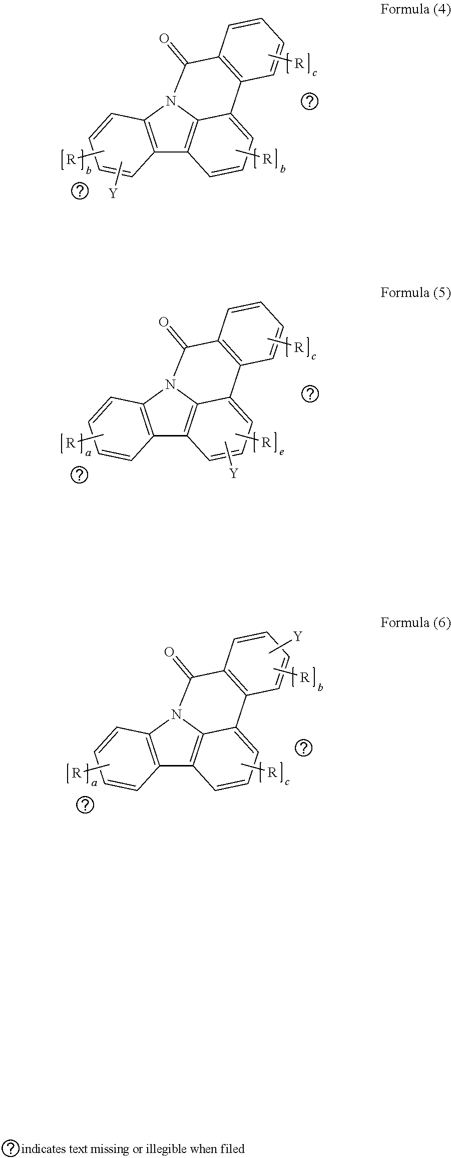

where the symbols and indices used have the definitions given above, and in addition:
e is 0, 1 or 2.

Preference is given to the compounds of the formula (4) or (5), particular preference to the compounds of the formula (4).

Preferred embodiments of the compounds of the formulae (4) to (6) are the compounds of the following formulae (4a) to (6a):

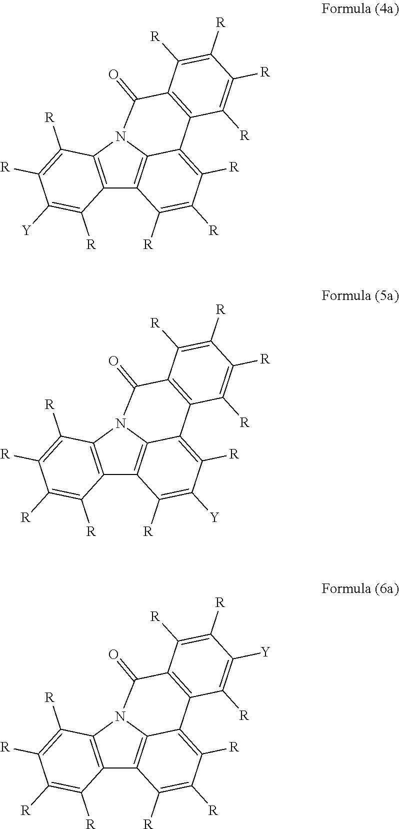

where the symbols used have the meanings given above.

Particular preference is given to the compounds of the following formulae (4b) to (6b):

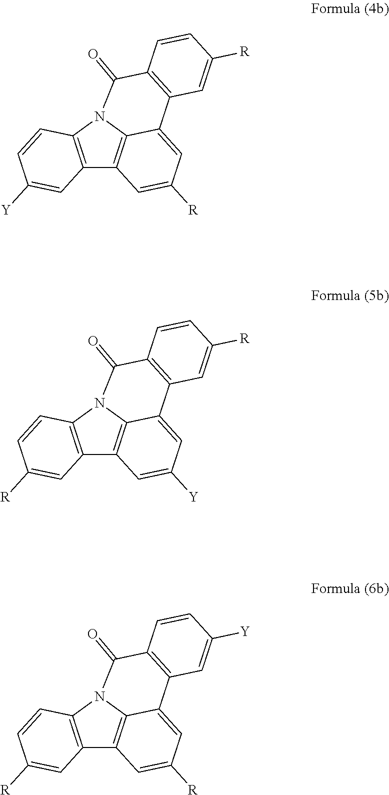

where the symbols used have the meanings given above.

In a further preferred embodiment of the invention, exactly one or two R radicals in the compound of the formula (1) are a Y group, and more preferably exactly one R radical is a Y group.

Preferred embodiments of the Y group are described hereinafter.

In a preferred embodiment of the invention, L is the same or different at each instance and is an aromatic or heteroaromatic ring system which has 6 to 24 aromatic ring atoms and may be substituted by one or more R′ radicals. When L is a heteroaromatic ring system, it preferably does not contain any electron-deficient heteroaryl groups, where the electron-deficient heteroaryl groups are characterized in that they contain a heteroaromatic six-membered ring having at least one nitrogen atom and/or an aromatic five-membered ring having at least two heteroatoms, at least one of which is a nitrogen atom, where aromatic or heteroaromatic groups may also be fused onto these groups. More preferably, L is the same or different at each instance and is an aromatic ring system having 6 to 24 aromatic ring atoms or is dibenzofuran, dibenzothiophene or carbazole, where these groups may each be substituted by one or more R′ radicals. Most preferably, L is the same or different at each instance and is an aromatic ring system having 6 to 12 aromatic ring atoms, especially phenylene, where these groups may each be substituted by one or more R′ radicals, but are preferably unsubstituted.

Examples of preferred L groups are the same or different at each instance and are selected from benzene, biphenyl, especially ortho-, meta- or para-biphenyl, terphenyl, especially ortho-terphenyl, meta-terphenyl, para-terphenyl or branched terphenyl, quaterphenyl, especially ortho-quaterphenyl, meta-quaterphenyl, para-quaterphenyl or branched quaterphenyl, fluorene, spirobifluorene, naphthalene, carbazole, dibenzofuran, dibenzothiophene, indenocarbazole, indolocarbazole, phenanthrene, triphenylene or a combination of two or three of these groups, each of which may be substituted by one or more R′ radicals, preferably nonaromatic R′ radicals.

Examples of suitable L groups are the structures of the following formulae (L-1) to (L-67), where the dotted bonds indicate the linkage of the group and the structures may each be substituted by one or more R′ radicals:

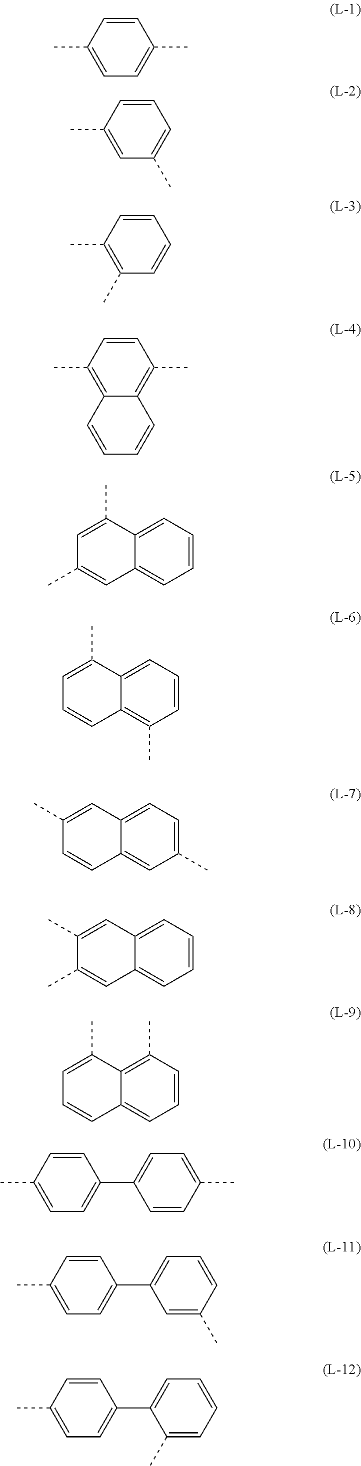

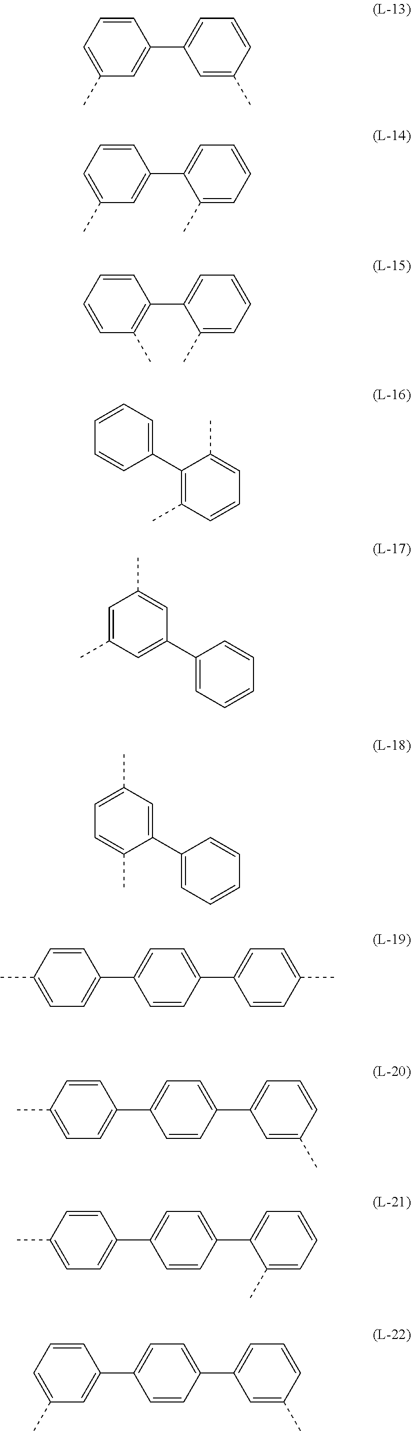

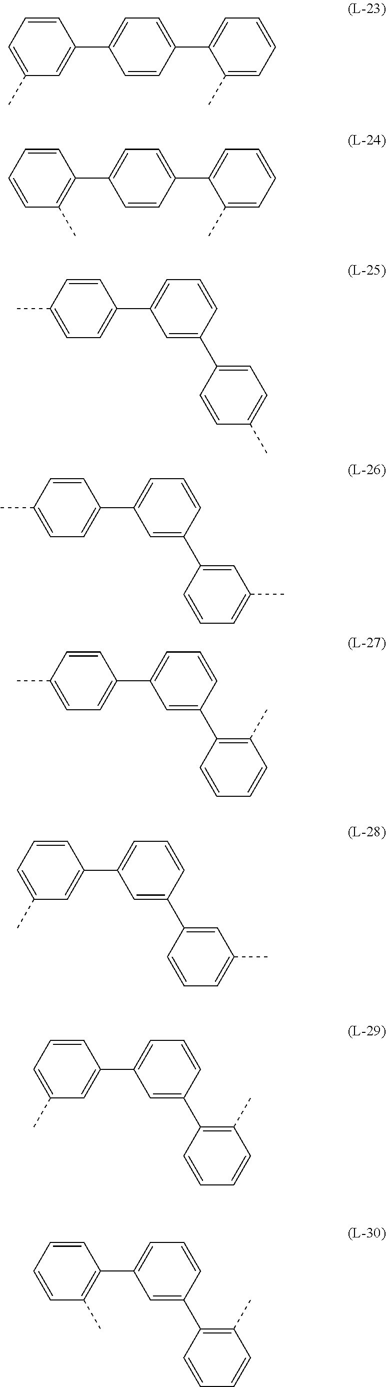

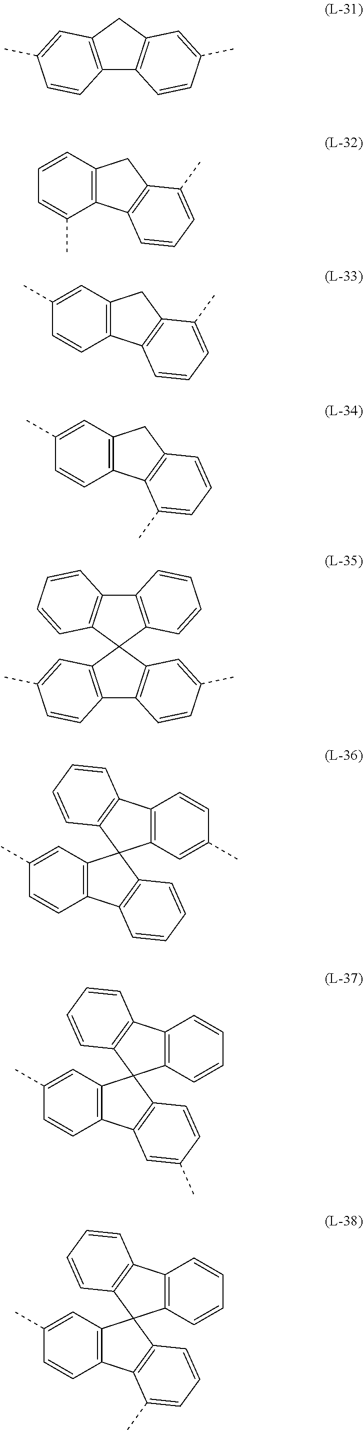

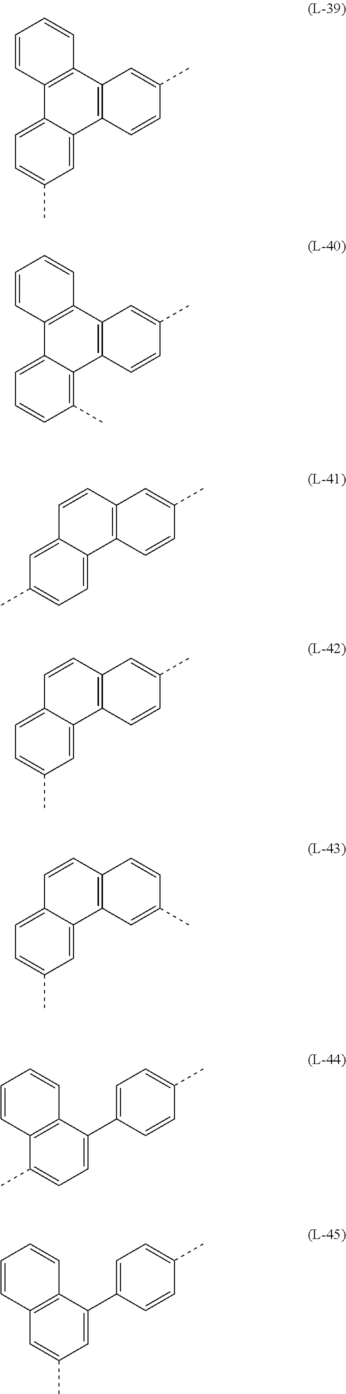

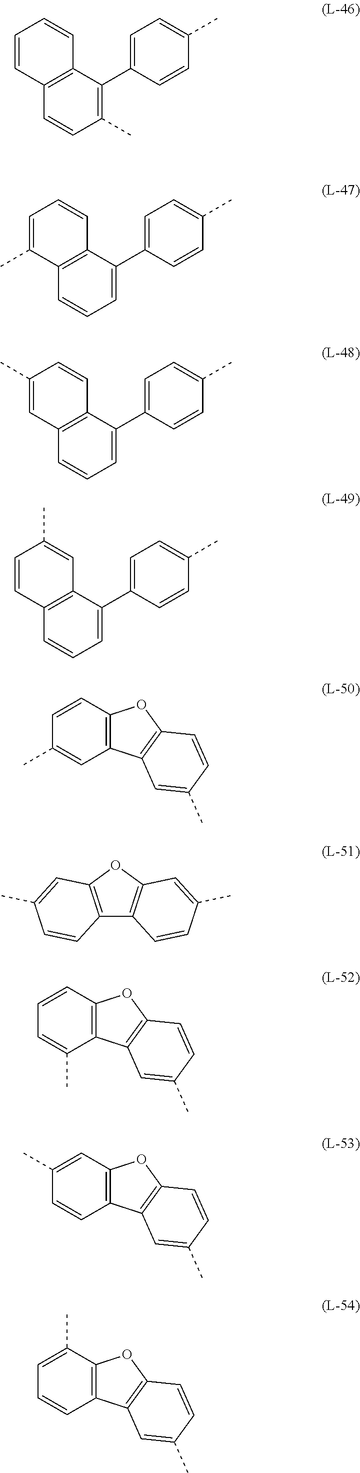

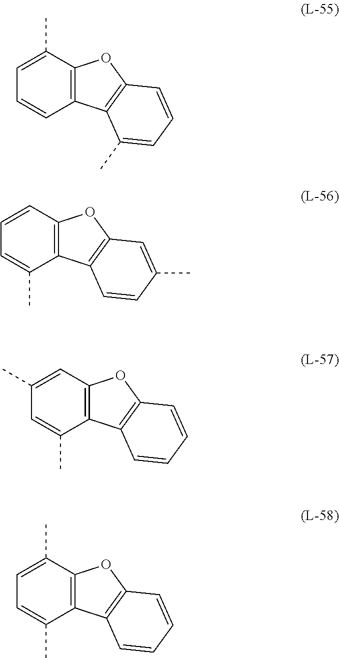

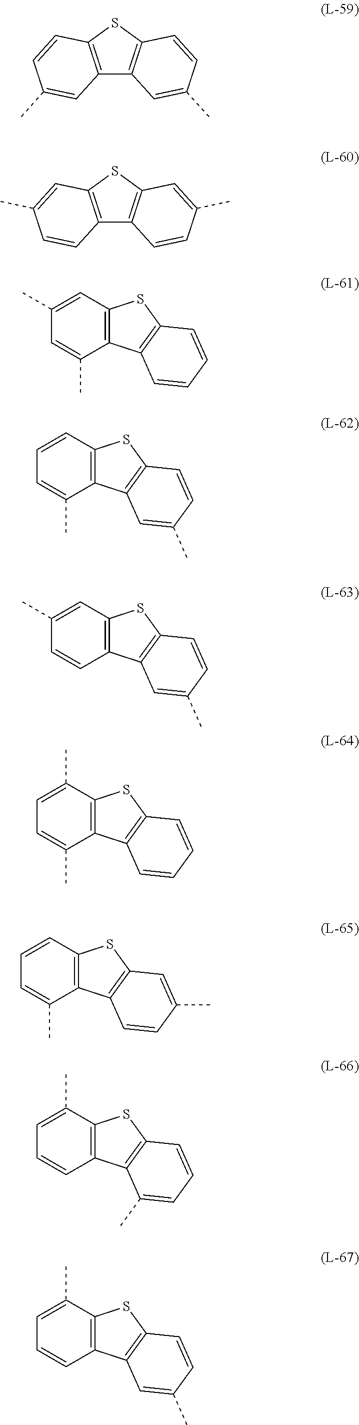

Particular preference is given to the structures (L-1) to (L-3), (L-10) to (L-18), (L-50), (L-52), (L-59) and (L-62).

The Y group is preferably selected from the groups of the following formulae (Y-1) to (Y-7):

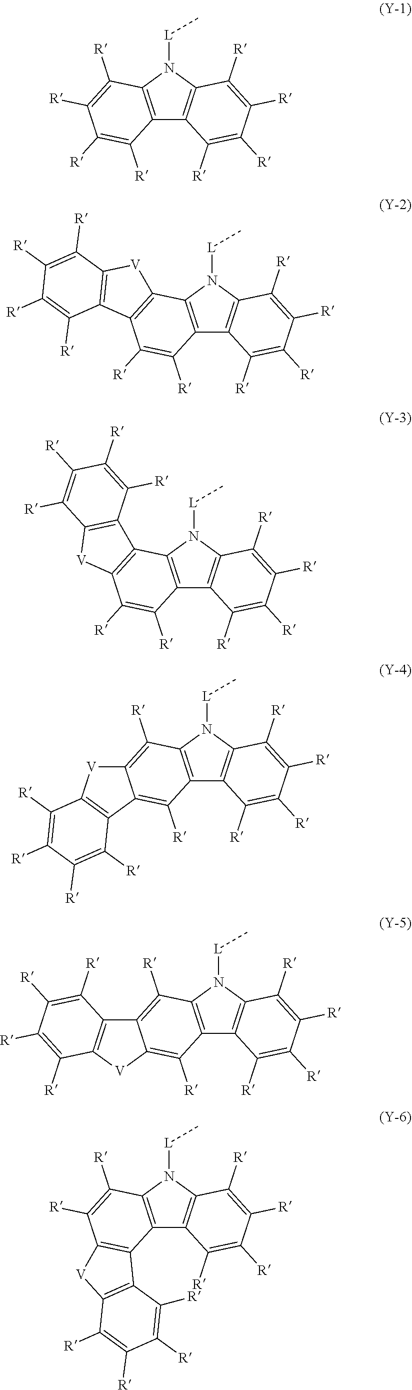

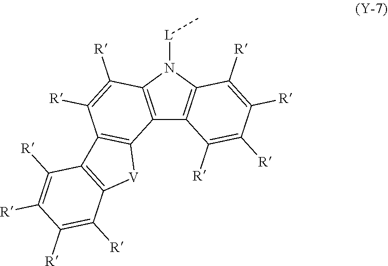

where the symbols used have the meanings given above.

More preferably, the Y group is selected from the structures of the following formulae (Y-1a) to (Y-7a):

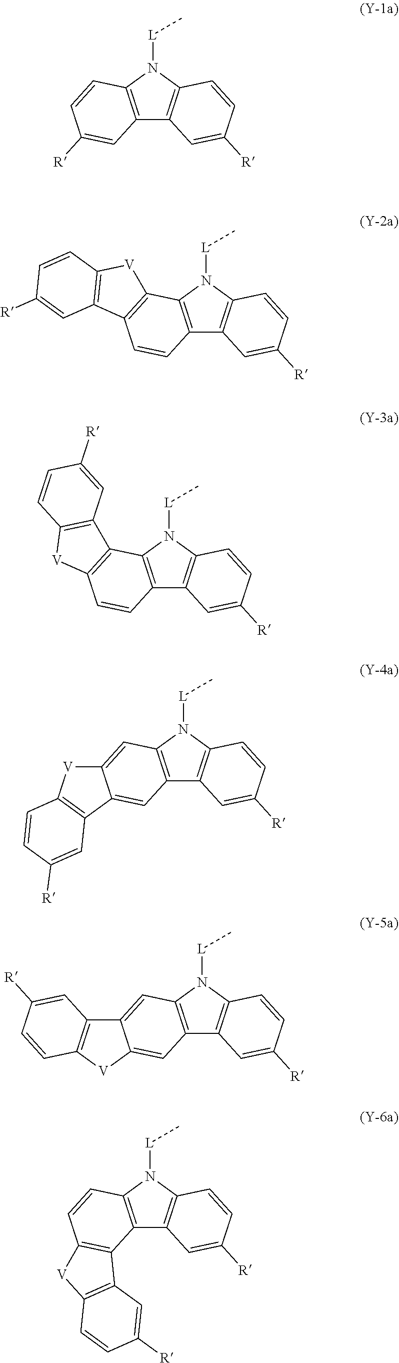

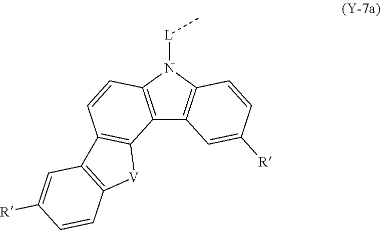

where the symbols used have the meanings given above.

In the formulae (Y-2) to (Y-7) and (Y-2a) to (Y-7a), all R′ radicals are preferably H.

In addition, in formula (Y-1) or (Y-1a), at least one R′ radical bonded in the para position to the nitrogen atom is preferably a group other than hydrogen. These R′ radicals here are preferably the same or different at each instance and are an aromatic or heteroaromatic ring system which has 6 to 24 aromatic ring atoms, more preferably 6 to 13 aromatic ring atoms, and may be substituted in each case by one or more R¹ radicals.

The group (Y-1) is more preferably a group of the following formula (Y-8), and the group (Y-1a) is more preferably a group of the following formula (Y-8a):

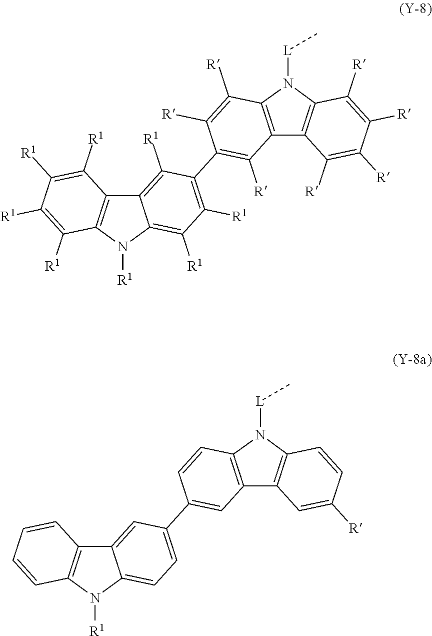

where the R¹ radical bonded to the nitrogen atom is preferably an aromatic or heteroaromatic ring system which has 6 to 30 aromatic ring atoms and may be substituted by one or more R² radicals, and where R′ in formula (Y-8a) is preferably H.

In a preferred embodiment of the invention, apart from the radical that represents the Y group, not more than three of the R and R′ radicals in total, more preferably not more than two R and R′ radicals in total and most preferably not more than one R or R′ radical in the compound of the formula (1) or the preferred structures detailed above are/is a group other than hydrogen.

There follows a description of preferred substituents R, R′, Ar′, R¹ and R² in the compounds of the invention. In a particularly preferred embodiment of the invention, the preferences specified hereinafter for R, R′, Ar′, R¹ and R² occur simultaneously and are applicable to the structures of the formula (1) and to all preferred embodiments detailed above.

In a preferred embodiment of the invention, R and R′ are the same or different at each instance and are selected from the group consisting of H, D, F, N(Ar′)₂, CN, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may each be substituted by one or more R¹ radicals, but is preferably unsubstituted, and where one or more nonadjacent CH₂ groups may be replaced by O, or an aromatic or heteroaromatic ring system which has 6 to 30 aromatic ring atoms and may be substituted in each case by one or more R¹ radicals; at the same time, two R radicals together may also form an aliphatic ring system; in addition, two R′ radicals together may also form an aliphatic or aromatic ring system. More preferably, R and R′ are the same or different at each instance and are selected from the group consisting of H, N(Ar′)₂, a straight-chain alkyl group having 1 to 6 carbon atoms, especially having 1, 2, 3 or 4 carbon atoms, or a branched or cyclic alkyl group having 3 to 6 carbon atoms, where the alkyl group in each case may be substituted by one or more R¹ radicals, but is preferably unsubstituted, or an aromatic or heteroaromatic ring system which has 6 to 24 aromatic ring atoms and may be substituted in each case by one or more R¹ radicals, preferably nonaromatic R¹ radicals. Most preferably, R and R′ are the same or different at each instance and are selected from the group consisting of H or an aromatic or heteroaromatic ring system which has 6 to 24 aromatic ring atoms, preferably 6 to 13 aromatic ring atoms, and may be substituted in each case by one or more R¹ radicals, preferably nonaromatic R¹ radicals. It may additionally be preferable when R or R′ is a triaryl- or -heteroarylamine group which may be substituted by one or more R¹ radicals. This group is one embodiment of an aromatic or heteroaromatic ring system, in which case two or more aryl or heteroaryl groups are joined to one another by a nitrogen atom. When R or R′ is a triaryl- or -heteroarylamine group, this group preferably has 18 to 30 aromatic ring atoms and may be substituted by one or more R¹ radicals, preferably nonaromatic R¹ radicals.

In a further preferred embodiment of the invention, Ar′ is an aromatic or heteroaromatic ring system which has 6 to 30 aromatic ring atoms and may be substituted by one or more R¹ radicals. In a particularly preferred embodiment of the invention, Ar′ is an aromatic or heteroaromatic ring system which has 6 to 24 aromatic ring atoms, especially 6 to 13 aromatic ring atoms, and may be substituted by one or more preferably nonaromatic R¹ radicals.

In a further preferred embodiment of the invention, R¹ is the same or different at each instance and is selected from the group consisting of H, D, F, CN, OR², a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may in each case be substituted by one or more R² radicals, and where one or more nonadjacent CH₂ groups may be replaced by O, or an aromatic or heteroaromatic ring system which has 6 to 30 aromatic ring atoms and may be substituted in each case by one or more R² radicals; at the same time, two or more R¹ radicals together may form an aliphatic ring system. In a particularly preferred embodiment of the invention, R¹ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R² radicals, but is preferably unsubstituted, or an aromatic or heteroaromatic ring system which has 6 to 24 aromatic ring atoms and may be substituted in each case by one or more R² radicals, but is preferably unsubstituted.

In a further preferred embodiment of the invention, R² is the same or different at each instance and is H, F, an alkyl group having 1 to 4 carbon atoms or an aryl group having 6 to 10 carbon atoms, which may be substituted by an alkyl group having 1 to 4 carbon atoms, but is preferably unsubstituted.

Suitable aromatic or heteroaromatic ring systems R, R′ or Ar′ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, quinazoline, benzimidazole, phenanthrene, triphenylene or a combination of two or three of these groups, each of which may be substituted by one or more R¹ radicals. When R or R′ or Ar′ is a heteroaryl group, especially triazine, pyrimidine, quinazoline or carbazole, preference may also be given to aromatic or heteroaromatic R¹ radicals on this heteroaryl group.

The R or R′ groups here, when they are an aromatic or heteroaromatic ring system, or Ar′ are preferably selected from the groups of the following formulae R-1 to R-83:

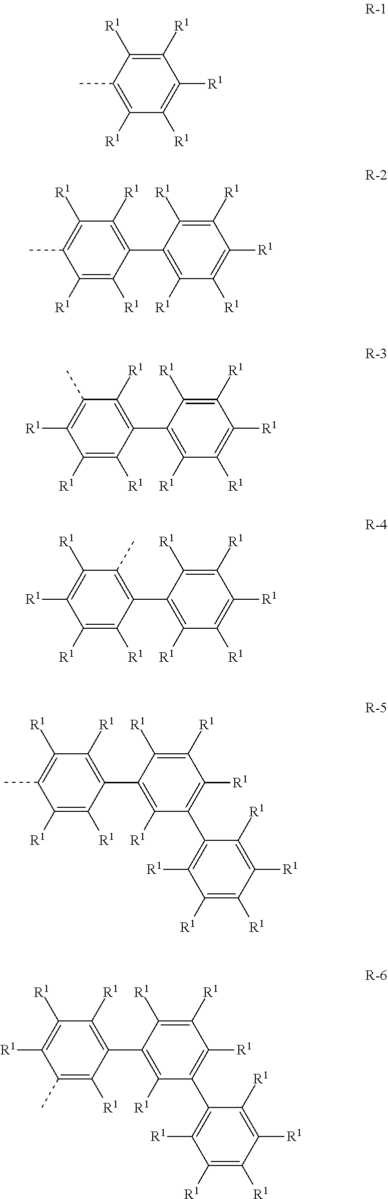

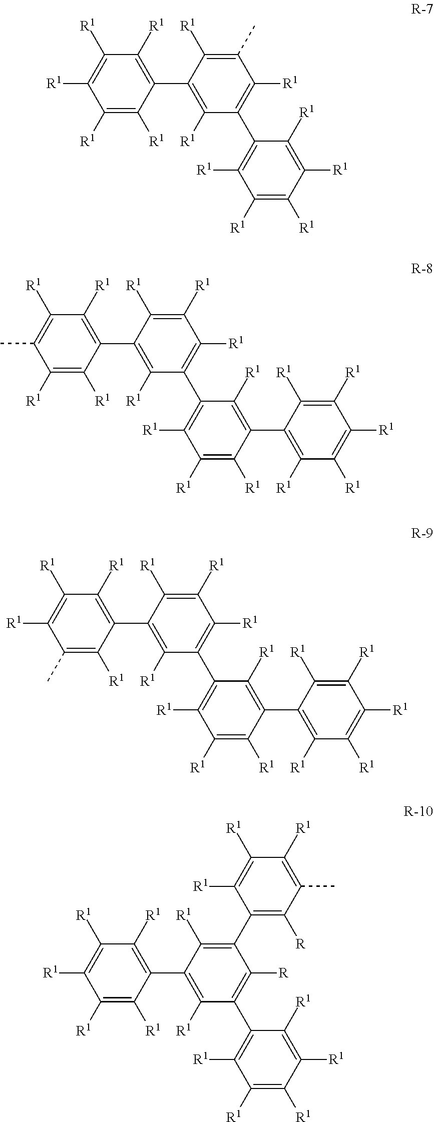

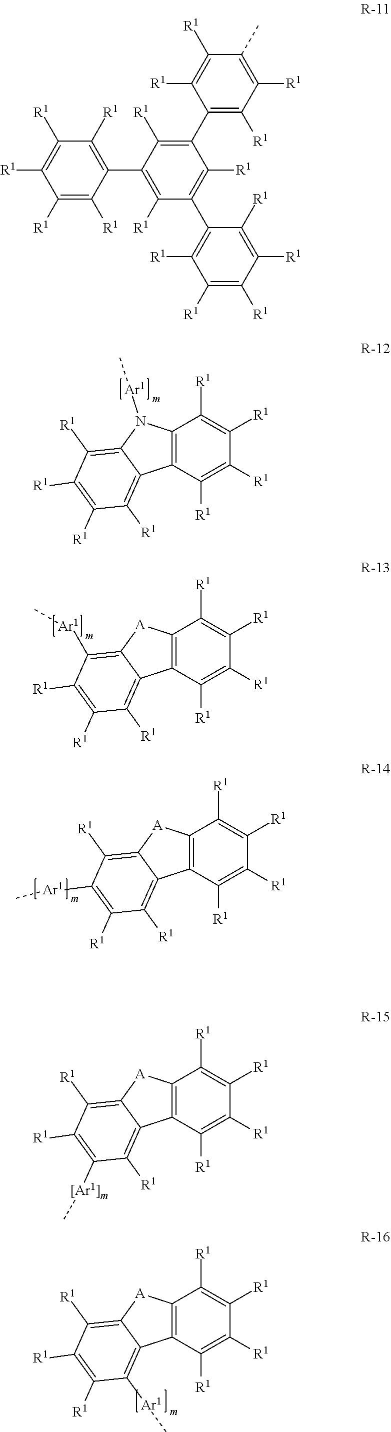

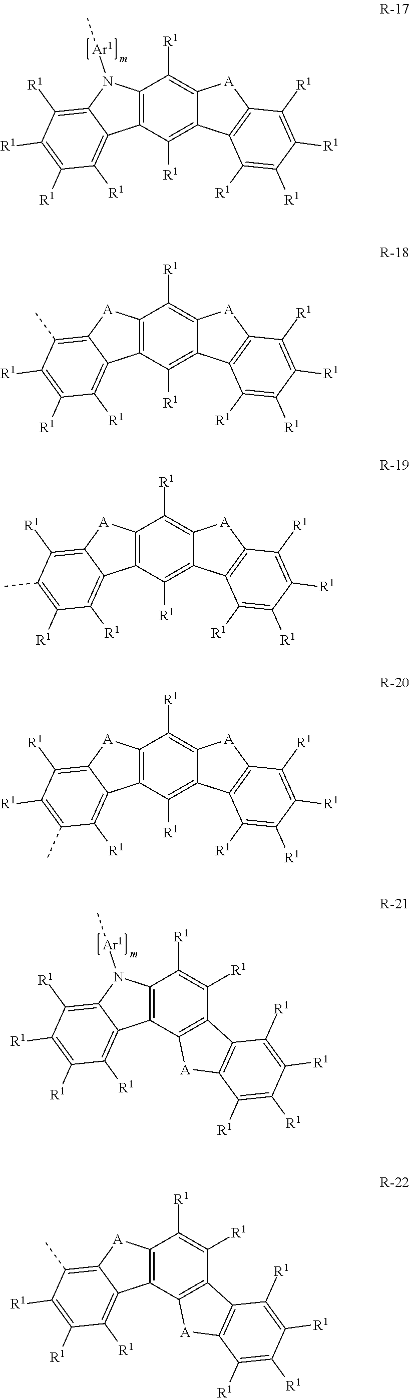

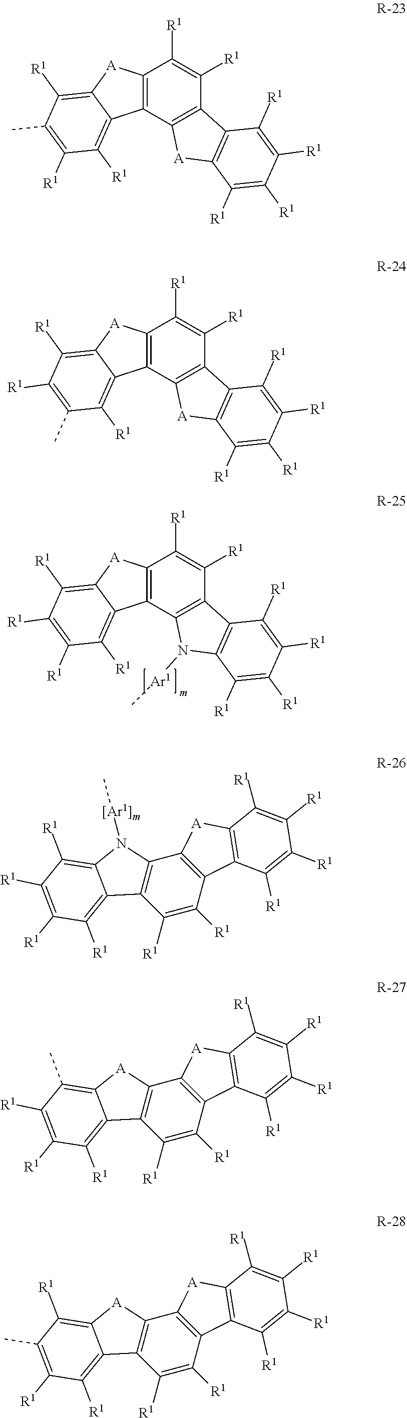

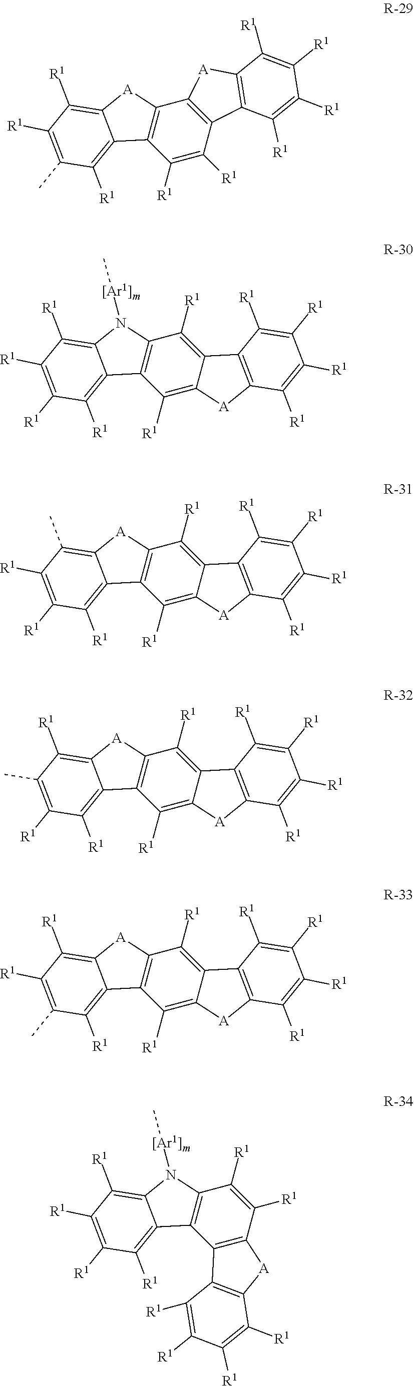

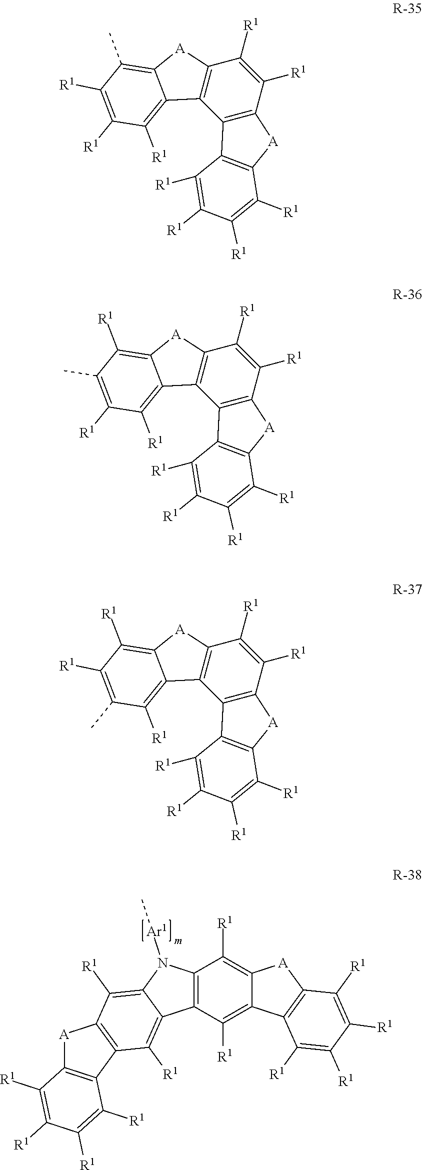

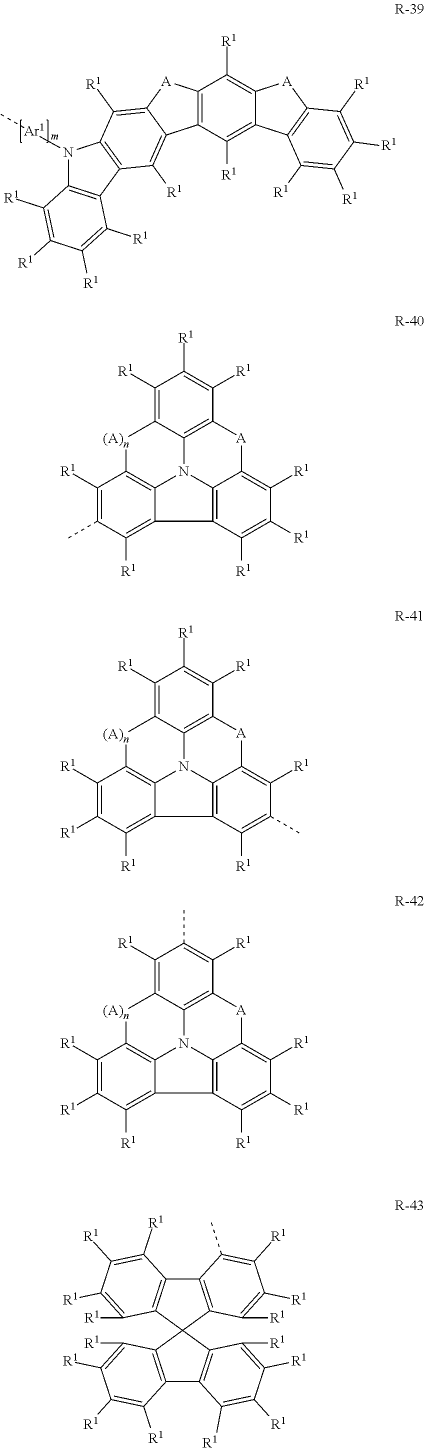

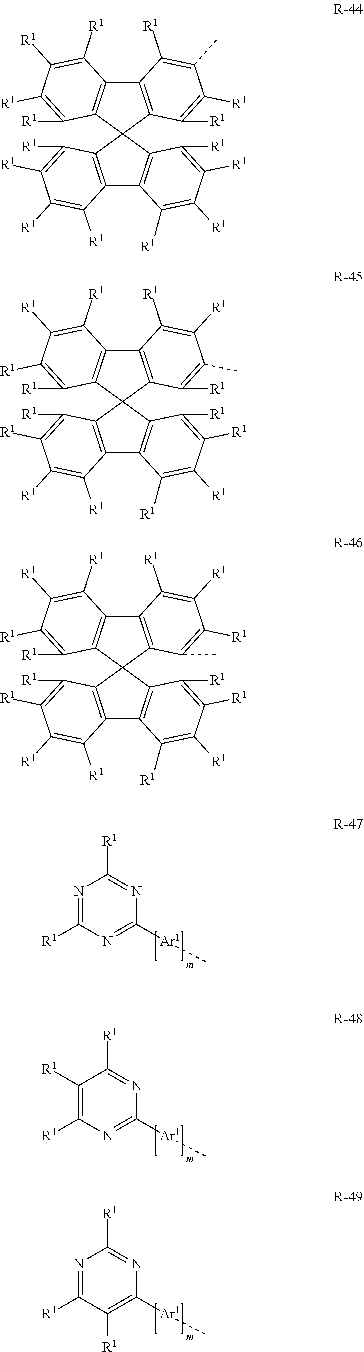

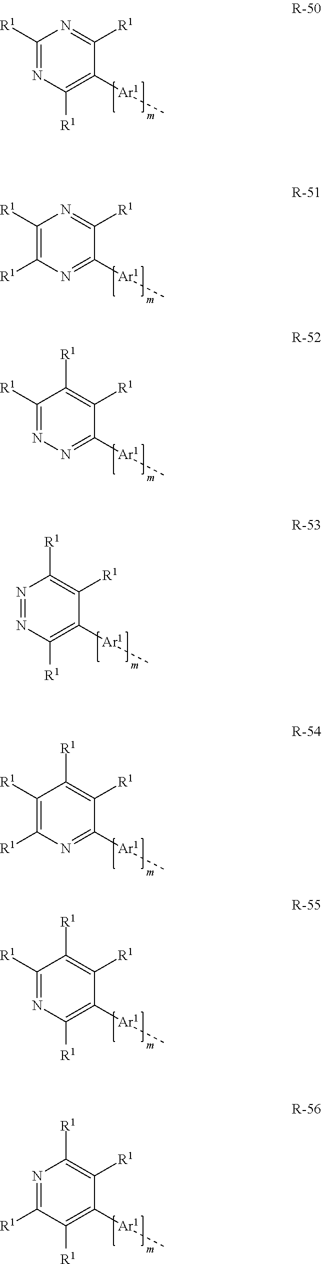

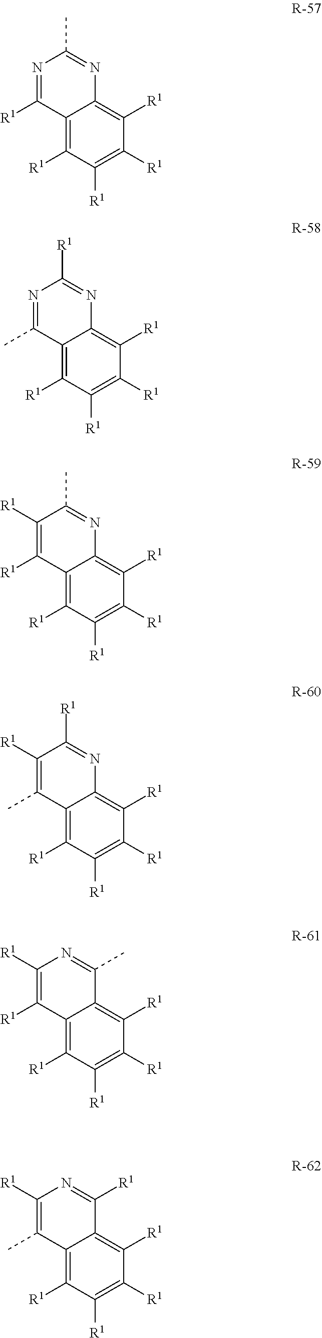

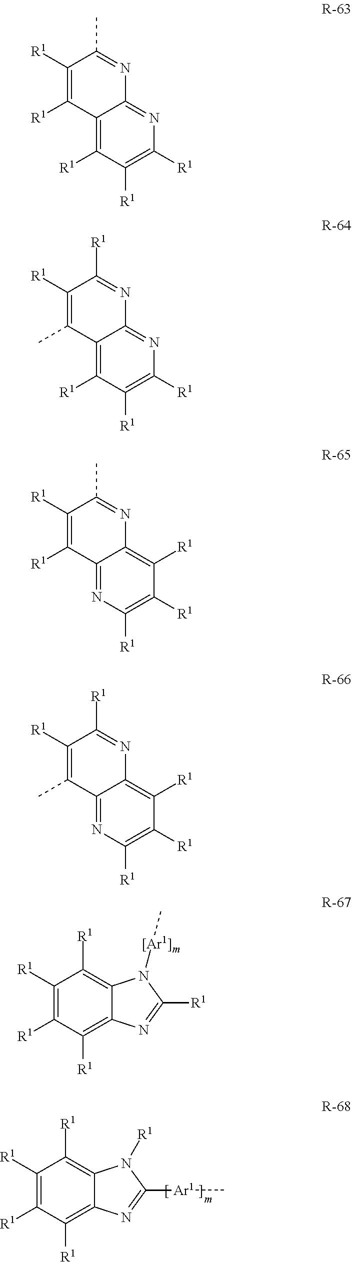

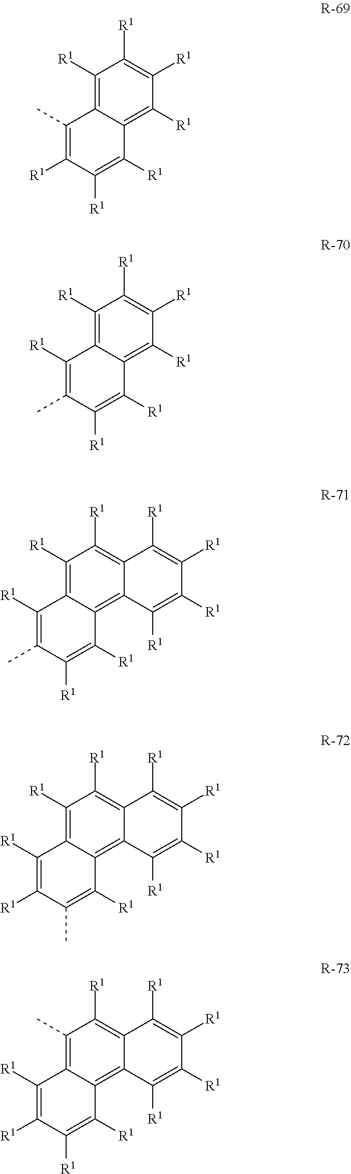

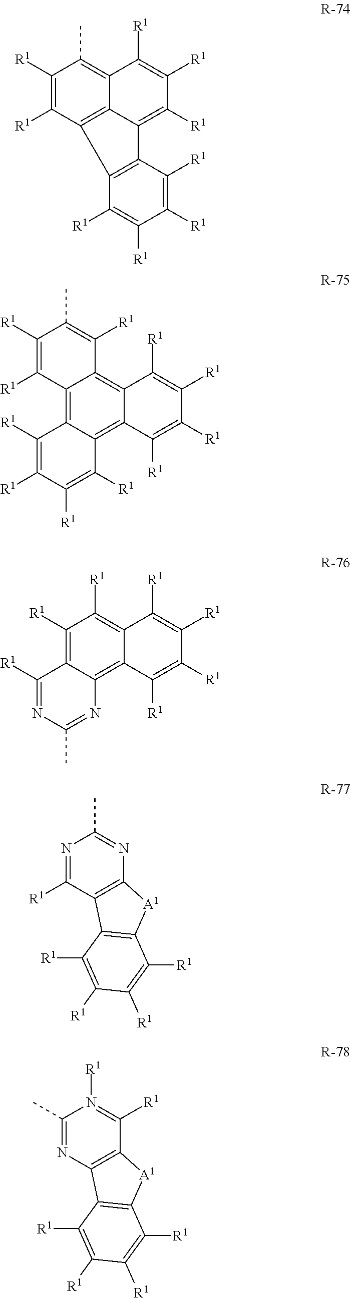

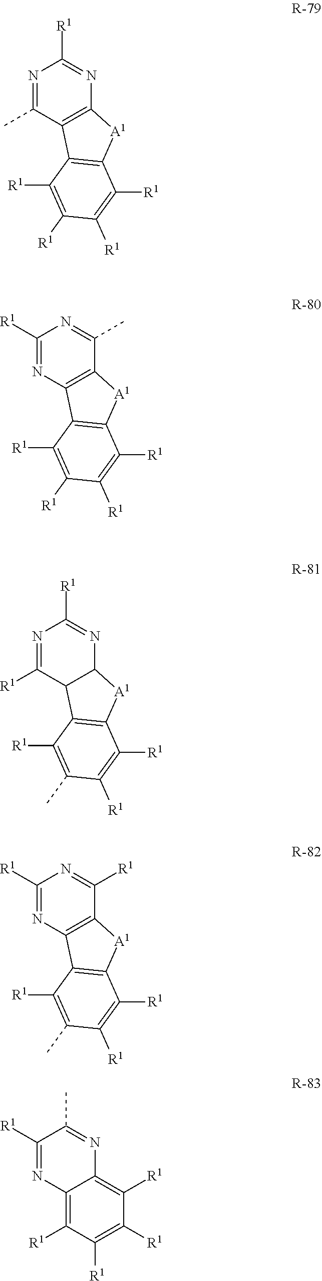

where R¹ has the definitions given above, the dotted bond represents the bond to a carbon atom of the base skeleton in formulae (1), (2) and (3) or in the preferred embodiments or to the nitrogen atom in the N(Ar′)₂ group and, in addition:
Ar¹ is the same or different at each instance and is a bivalent aromatic or heteroaromatic ring system which has 6 to 18 aromatic ring atoms and may be substituted in each case by one or more R¹ radicals;
A is the same or different at each instance and is C(R¹)₂, NR¹, O or S;
n is 0 or 1, where n=0 means that no A group is bonded at this position and R¹ radicals are bonded to the corresponding carbon atoms instead;
m is 0 or 1, where m=0 means that the Ar^{t} group is absent and that the corresponding aromatic or heteroaromatic group is bonded directly to a carbon atom of the base skeleton in formula (1) or in the preferred embodiments, or to the nitrogen atom in the N(Ar′)₂ group; with the proviso that m=1 for the structures (R-12), (R-17), (R-21), (R-25), (R-26), (R-30), (R-34), (R-38) and (R-39) when these groups are embodiments of Ar′.

When the abovementioned R-1 to R-83 groups for R, R′ or Ar′ have two or more A groups, possible options for these include all combinations from the definition of A. Preferred embodiments in that case are those in which one A group is NR¹ and the other A group is C(R¹)₂ or in which both A groups are NR¹ or in which both A groups are O. In a particularly preferred embodiment of the invention, in Ar, R or Ar′ groups having two or more A groups, at least one A group is C(R¹)₂ or is NR¹.

When A is NR¹, the substituent R¹ bonded to the nitrogen atom is preferably an aromatic or heteroaromatic ring system which has 5 to 24 aromatic ring atoms and may also be substituted by one or more R² radicals. In a particularly preferred embodiment, this R¹ substituent is the same or different at each instance and is an aromatic or heteroaromatic ring system which has 6 to 24 aromatic ring atoms, preferably 6 to 12 aromatic ring atoms, and which does not have any fused aryl groups or heteroaryl groups in which two or more aromatic or heteroaromatic 6-membered ring groups are fused directly to one another, and which may also be substituted in each case by one or more R² radicals. Particular preference is given to phenyl, biphenyl, terphenyl and quaterphenyl having bonding patterns as listed above for R-1 to R-11, where these structures may be substituted by one or more R² radicals, but are preferably unsubstituted.

When A is C(R¹)₂, the substituents R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R² radicals. Most preferably, R¹ is a methyl group or a phenyl group. In this case, the R¹ radicals together may also form a ring system, which leads to a spiro system.

Further suitable R, R′ or Ar′ groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R¹ radicals. The total number of aromatic ring atoms in Ar², Ar³ and Ar⁴ here is not more than 60 and preferably not more than 40.

In this case, Ar⁴ and Ar² may also be bonded to one another and/or Ar² and Ar³ to one another via a group selected from C(R¹)₂, NR¹, O and S. Preferably, Ar⁴ and Ar² are joined to one another and Ar² and Ar³ to one another in the respective ortho position to the bond to the nitrogen atom. In a further embodiment of the invention, none of the Ar², Ar³ and Ar⁴ groups are bonded to one another.

Preferably, Ar⁴ is an aromatic or heteroaromatic ring system which has 6 to 24 aromatic ring atoms, especially 6 to 12 aromatic ring atoms, and may be substituted in each case by one or more R¹ radicals. More preferably, Ar⁴ is selected from the group consisting of ortho-, meta- or para-phenylene or ortho-, meta- or para-biphenyl, each of which may be substituted by one or more R¹ radicals, but are preferably unsubstituted. Most preferably, Ar⁴ is an unsubstituted phenylene group. This is especially true when Ar⁴ is bonded to Ar² via a single bond.

Preferably, Ar² and Ar³ are the same or different at each instance and are an aromatic or heteroaromatic ring system which has 6 to 24 aromatic ring atoms and may be substituted in each case by one or more R¹ radicals.

Particularly preferred Ar² and Ar³ groups are the same or different at each instance and are selected from the group consisting of benzene, ortho-, meta- or para-biphenyl, ortho-, meta- or para-terphenyl or branched terphenyl, ortho-, meta- or para-quaterphenyl or branched quaterphenyl, 1-, 2-, 3- or 4-fluorenyl, 1-, 2-, 3- or 4-spirobifluorenyl, 1- or 2-naphthyl, indole, benzofuran, benzothiophene, 1-, 2-, 3- or 4-carbazole, 1-, 2-, 3- or 4-dibenzofuran, 1-, 2-, 3- or 4-dibenzothiophene, indenocarbazole, indolocarbazole, 2-, 3- or 4-pyridine, 2-, 4- or 5-pyrimidine, pyrazine, pyridazine, triazine, phenanthrene, triphenylene or combinations of two, three or four of these groups, each of which may be substituted by one or more R¹ radicals. More preferably, Ar² and Ar³ are the same or different at each instance and are an aromatic ring system which has 6 to 24 aromatic ring atoms and may be substituted by one or more R¹ radicals, especially selected from the groups consisting of benzene, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene, especially 1-, 2-, 3- or 4-fluorene, or spirobifluorene, especially 1-, 2-, 3- or 4-spirobifluorene.

At the same time, the alkyl groups in compounds that are processed by vacuum evaporation preferably have not more than five carbon atoms, more preferably not more than 4 carbon atoms, most preferably not more than 1 carbon atom. For compounds which are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

When the compounds of the formula (1) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. It is especially preferable when the R, R′, Ar′, R¹ and R² radicals do not contain any fused aryl or heteroaryl groups in which two or more six-membered rings are fused directly to one another. An exception to this is formed by phenanthrene, triphenylene and quinazoline, which, because of their high triplet energy, may be preferable in spite of the presence of fused aromatic six-membered rings.

The abovementioned preferred embodiments may be combined with one another as desired within the restrictions defined in claim 1. In a particularly preferred embodiment of the invention, the abovementioned preferences occur simultaneously.

Examples of preferred compounds according to the embodiments detailed above are the compounds detailed in the following table:

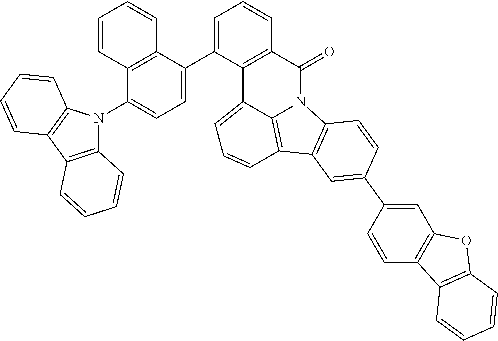 [SMILES: O=c1c2cccc(-c3ccc(-n4c5ccccc5c5ccccc54)c4ccccc34)c2c2cccc3c4cc(-c5ccc6c(c5)oc5ccccc56)ccc4n1c32]

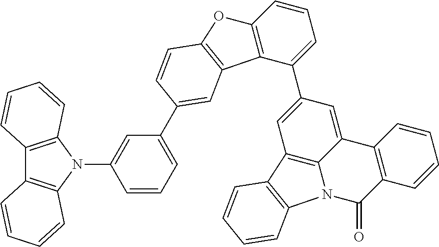 [SMILES: O=c1c2ccccc2c2cc(-c3cccc4oc5ccc(-c6cccc(-n7c8ccccc8c8ccccc87)c6)cc5c34)cc3c4ccccc4n1c23]

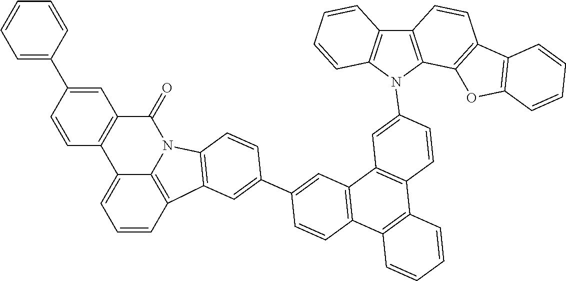 [SMILES: O=c1c2cc(-c3ccccc3)ccc2c2cccc3c4cc(-c5ccc6c7ccccc7c7ccc(-n8c9ccccc9c9ccc%10c%11ccccc%11oc%10c98)cc7c6c5)ccc4n1c23]

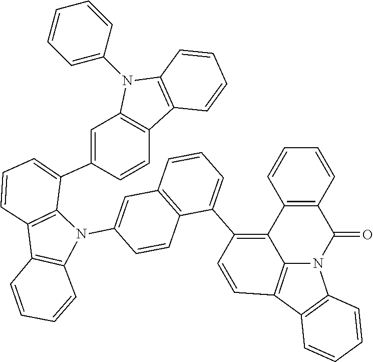 [SMILES: O=c1c2ccccc2c2c(-c3cccc4cc(-n5c6ccccc6c6cccc(-c7ccc8c9ccccc9n(-c9ccccc9)c8c7)c65)ccc34)ccc3c4ccccc4n1c32]

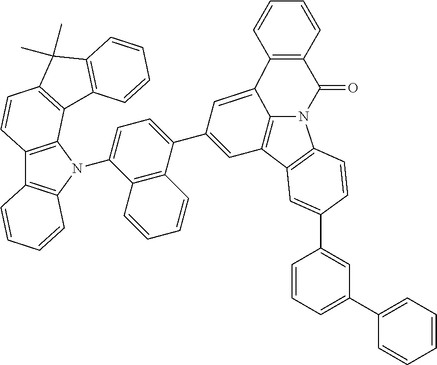 [SMILES: CC1(C)c2ccccc2-c2c1ccc1c3ccccc3n(-c3ccc(-c4cc5c6ccccc6c(=O)n6c7ccc(-c8cccc(-c9ccccc9)c8)cc7c(c4)c56)c4ccccc34)c21]

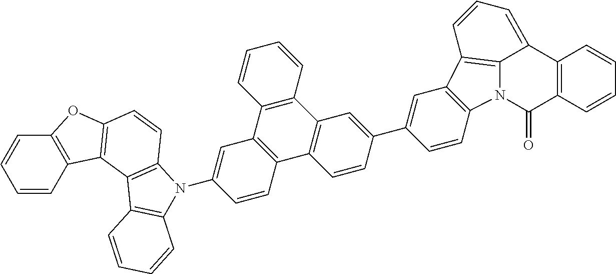 [SMILES: O=c1c2ccccc2c2cccc3c4cc(-c5ccc6c7ccc(-n8c9ccccc9c9c%10c(ccc98)oc8ccccc8%10)cc7c7ccccc7c6c5)ccc4n1c23]

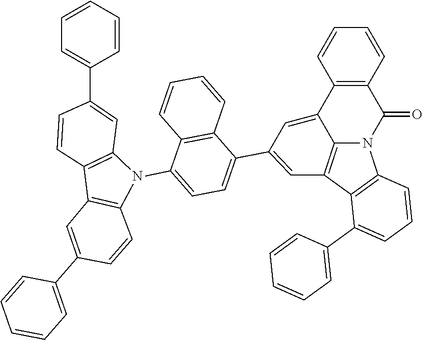 [SMILES: O=c1c2ccccc2c2cc(-c3ccc(-n4c5ccc(-c6ccccc6)cc5c5ccc(-c6ccccc6)cc54)c4ccccc34)cc3c4c(-c5ccccc5)cccc4n1c23]

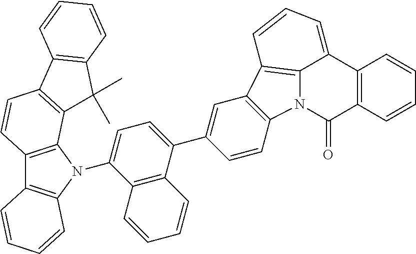 [SMILES: CC1(C)c2ccccc2-c2ccc3c4ccccc4n(-c4ccc(-c5ccc6c(c5)c5cccc7c8ccccc8c(=O)n6c75)c5ccccc45)c3c21]

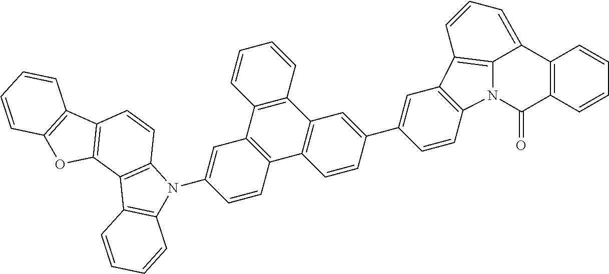 [SMILES: O=c1c2ccccc2c2cccc3c4cc(-c5ccc6c7ccc(-n8c9ccccc9c9c%10oc%11ccccc%11c%10ccc98)cc7c7ccccc7c6c5)ccc4n1c23]

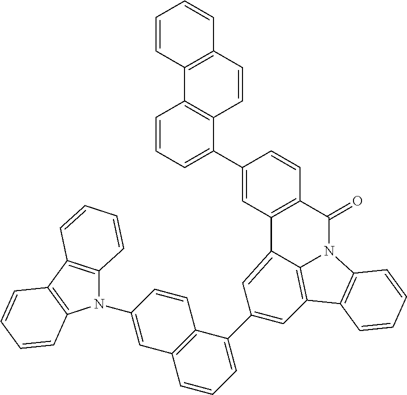 [SMILES: O=c1c2ccc(-c3cccc4c3ccc3ccccc34)cc2c2cc(-c3cccc4cc(-n5c6ccccc6c6ccccc65)ccc34)cc3c4ccccc4n1c23]

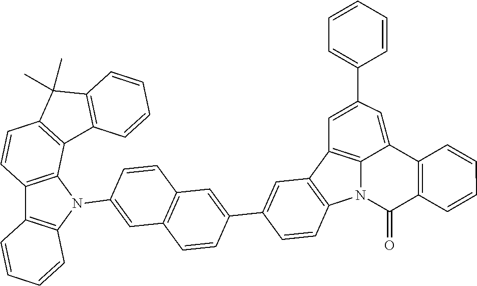 [SMILES: CC1(C)c2ccccc2-c2c1ccc1c3ccccc3n(-c3ccc4cc(-c5ccc6c(c5)c5cc(-c7ccccc7)cc7c8ccccc8c(=O)n6c75)ccc4c3)c21]

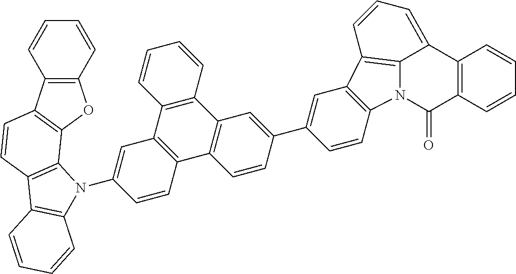 [SMILES: O=c1c2ccccc2c2cccc3c4cc(-c5ccc6c7ccc(-n8c9ccccc9c9ccc%10c%11ccccc%11oc%10c98)cc7c7ccccc7c6c5)ccc4n1c23]

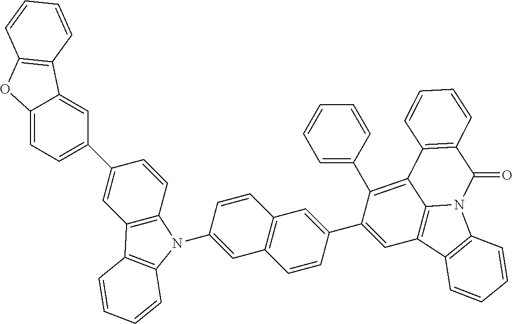 [SMILES: O=c1c2ccccc2c2c(-c3ccccc3)c(-c3ccc4cc(-n5c6ccccc6c6cc(-c7ccc8oc9ccccc9c8c7)ccc65)ccc4c3)cc3c4ccccc4n1c32]

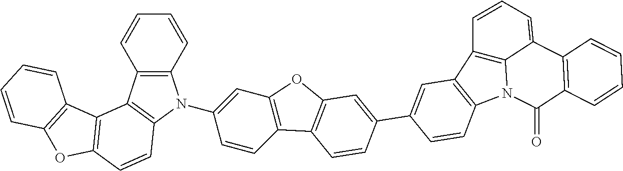 [SMILES: O=c1c2ccccc2c2cccc3c4cc(-c5ccc6c(c5)oc5cc(-n7c8ccccc8c8c9c(ccc87)oc7ccccc79)ccc56)ccc4n1c23]

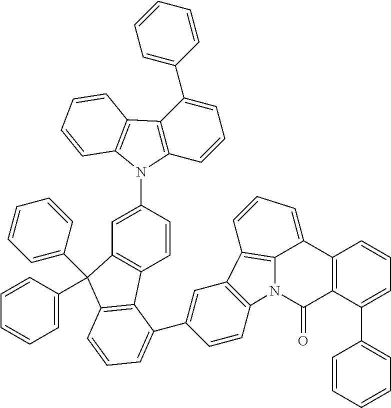 [SMILES: O=c1c2c(-c3ccccc3)cccc2c2cccc3c4cc(-c5cccc6c5-c5ccc(-n7c8ccccc8c8c(-c9ccccc9)cccc87)cc5C6(c5ccccc5)c5ccccc5)ccc4n1c23]

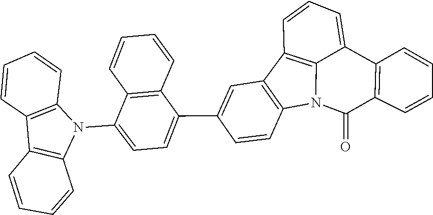 [SMILES: O=c1c2ccccc2c2cccc3c4cc(-c5ccc(-n6c7ccccc7c7ccccc76)c6ccccc56)ccc4n1c23]

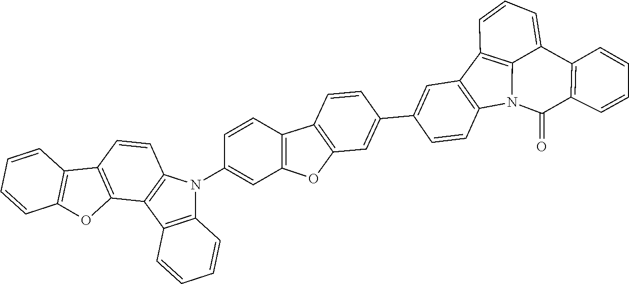 [SMILES: O=c1c2ccccc2c2cccc3c4cc(-c5ccc6c(c5)oc5cc(-n7c8ccccc8c8c9oc%10ccccc%10c9ccc87)ccc56)ccc4n1c23]

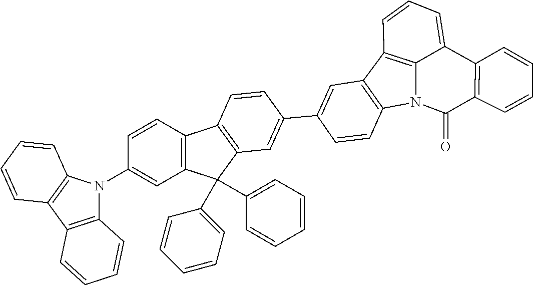 [SMILES: O=c1c2ccccc2c2cccc3c4cc(-c5ccc6c(c5)C(c5ccccc5)(c5ccccc5)c5cc(-n7c8ccccc8c8ccccc87)ccc5-6)ccc4n1c23]

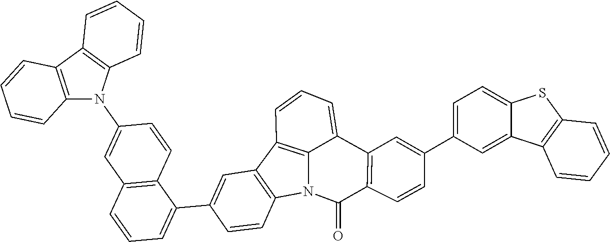 [SMILES: O=c1c2ccc(-c3ccc4sc5ccccc5c4c3)cc2c2cccc3c4cc(-c5cccc6cc(-n7c8ccccc8c8ccccc87)ccc56)ccc4n1c23]

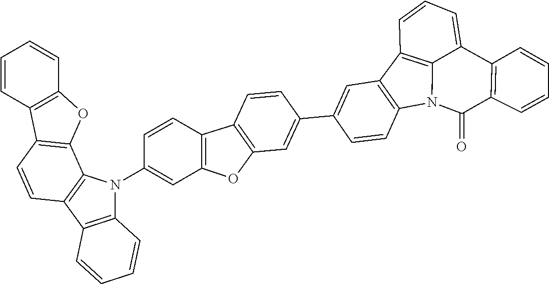 [SMILES: O=c1c2ccccc2c2cccc3c4cc(-c5ccc6c(c5)oc5cc(-n7c8ccccc8c8ccc9c%10ccccc%10oc9c87)ccc56)ccc4n1c23]

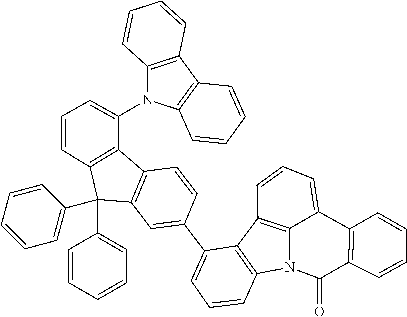 [SMILES: O=c1c2ccccc2c2cccc3c4c(-c5ccc6c(c5)C(c5ccccc5)(c5ccccc5)c5cccc(-n7c8ccccc8c8ccccc87)c5-6)cccc4n1c23]

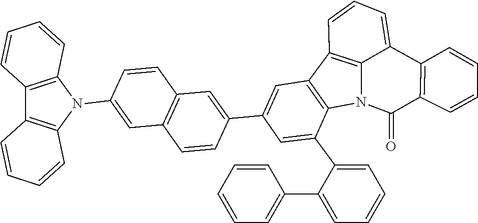 [SMILES: O=c1c2ccccc2c2cccc3c4cc(-c5ccc6cc(-n7c8ccccc8c8ccccc87)ccc6c5)cc(-c5ccccc5-c5ccccc5)c4n1c23]

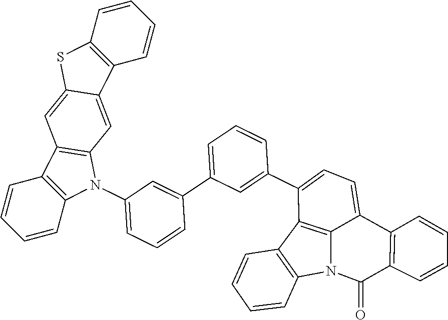 [SMILES: O=c1c2ccccc2c2ccc(-c3cccc(-c4cccc(-n5c6ccccc6c6cc7sc8ccccc8c7cc65)c4)c3)c3c4ccccc4n1c23]

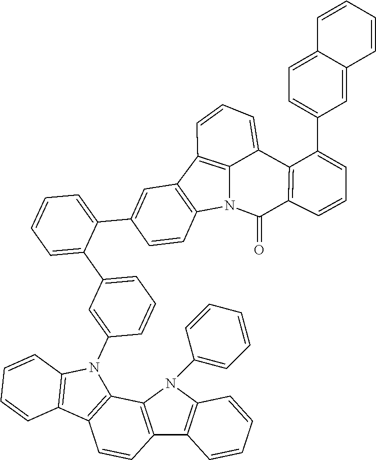 [SMILES: O=c1c2cccc(-c3ccc4ccccc4c3)c2c2cccc3c4cc(-c5ccccc5-c5cccc(-n6c7ccccc7c7ccc8c9ccccc9n(-c9ccccc9)c8c76)c5)ccc4n1c32]

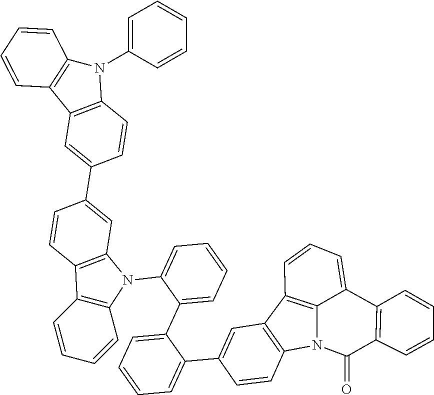 [SMILES: O=c1c2ccccc2c2cccc3c4cc(-c5ccccc5-c5ccccc5-n5c6ccccc6c6ccc(-c7ccc8c(c7)c7ccccc7n8-c7ccccc7)cc65)ccc4n1c23]

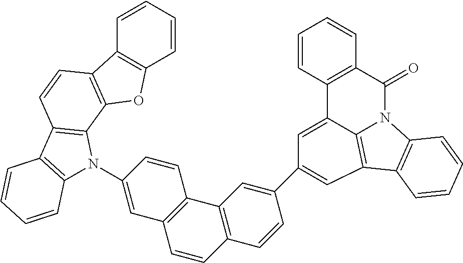 [SMILES: O=c1c2ccccc2c2cc(-c3ccc4ccc5cc(-n6c7ccccc7c7ccc8c9ccccc9oc8c76)ccc5c4c3)cc3c4ccccc4n1c23]

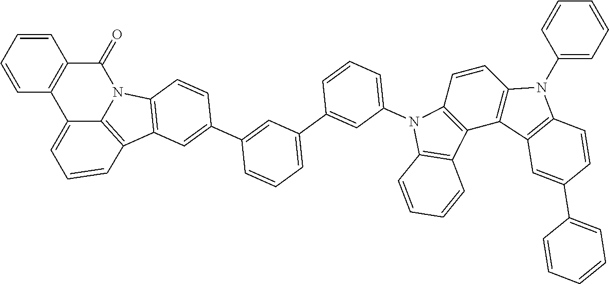 [SMILES: O=c1c2ccccc2c2cccc3c4cc(-c5cccc(-c6cccc(-n7c8ccccc8c8c9c%10cc(-c%11ccccc%11)ccc%10n(-c%10ccccc%10)c9ccc87)c6)c5)ccc4n1c23]

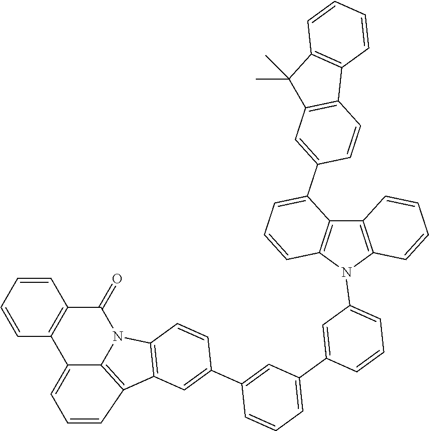 [SMILES: CC1(C)c2ccccc2-c2ccc(-c3cccc4c3c3ccccc3n4-c3cccc(-c4cccc(-c5ccc6c(c5)c5cccc7c8ccccc8c(=O)n6c75)c4)c3)cc21]

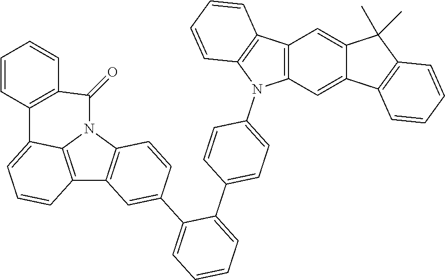 [SMILES: CC1(C)c2ccccc2-c2cc3c(cc21)c1ccccc1n3-c1ccc(-c2ccccc2-c2ccc3c(c2)c2cccc4c5ccccc5c(=O)n3c42)cc1]

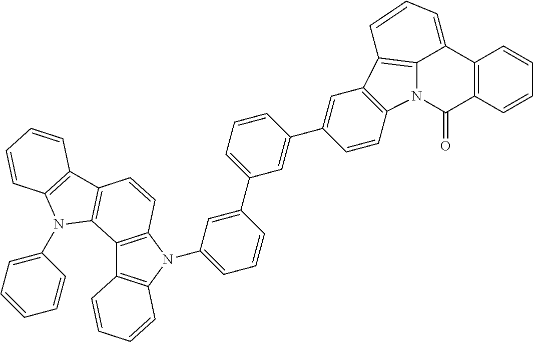 [SMILES: O=c1c2ccccc2c2cccc3c4cc(-c5cccc(-c6cccc(-n7c8ccccc8c8c7ccc7c9ccccc9n(-c9ccccc9)c78)c6)c5)ccc4n1c23]

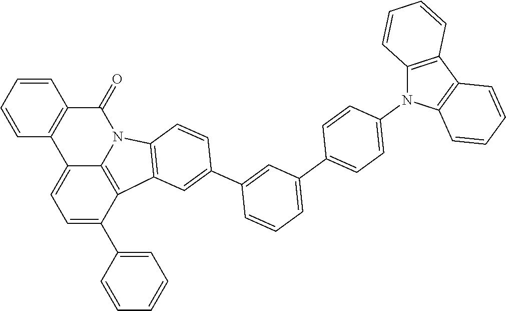 [SMILES: O=c1c2ccccc2c2ccc(-c3ccccc3)c3c4cc(-c5cccc(-c6ccc(-n7c8ccccc8c8ccccc87)cc6)c5)ccc4n1c23]

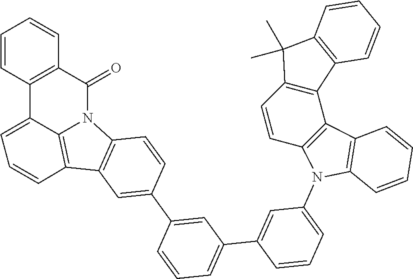 [SMILES: CC1(C)c2ccccc2-c2c1ccc1c2c2ccccc2n1-c1cccc(-c2cccc(-c3ccc4c(c3)c3cccc5c6ccccc6c(=O)n4c53)c2)c1]

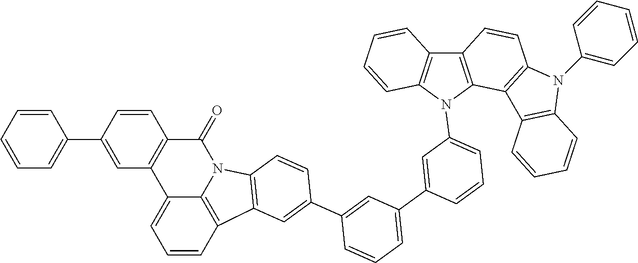 [SMILES: O=c1c2ccc(-c3ccccc3)cc2c2cccc3c4cc(-c5cccc(-c6cccc(-n7c8ccccc8c8ccc9c(c%10ccccc%10n9-c9ccccc9)c87)c6)c5)ccc4n1c23]

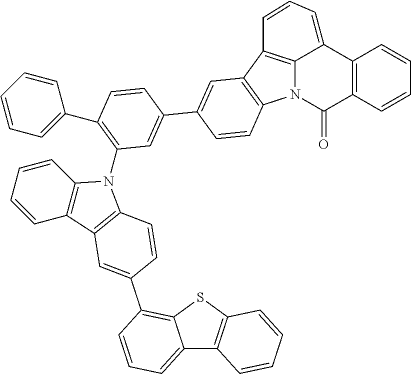 [SMILES: O=c1c2ccccc2c2cccc3c4cc(-c5ccc(-c6ccccc6)c(-n6c7ccccc7c7cc(-c8cccc9c8sc8ccccc89)ccc76)c5)ccc4n1c23]

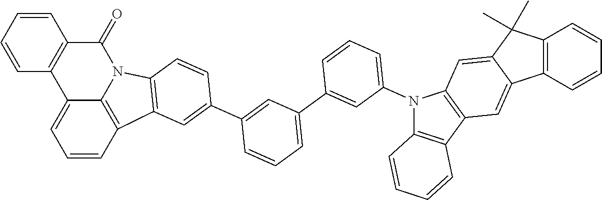 [SMILES: CC1(C)c2ccccc2-c2cc3c4ccccc4n(-c4cccc(-c5cccc(-c6ccc7c(c6)c6cccc8c9ccccc9c(=O)n7c86)c5)c4)c3cc21]

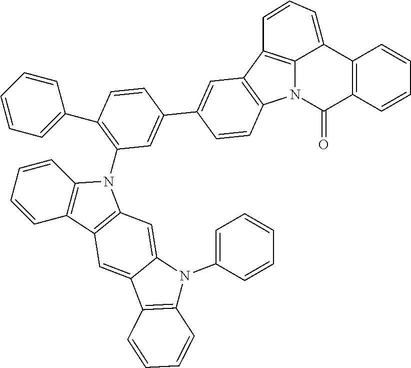 [SMILES: O=c1c2ccccc2c2cccc3c4cc(-c5ccc(-c6ccccc6)c(-n6c7ccccc7c7cc8c9ccccc9n(-c9ccccc9)c8cc76)c5)ccc4n1c23]

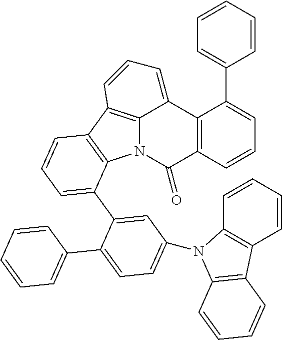 [SMILES: O=c1c2cccc(-c3ccccc3)c2c2cccc3c4cccc(-c5cc(-n6c7ccccc7c7ccccc76)ccc5-c5ccccc5)c4n1c32]

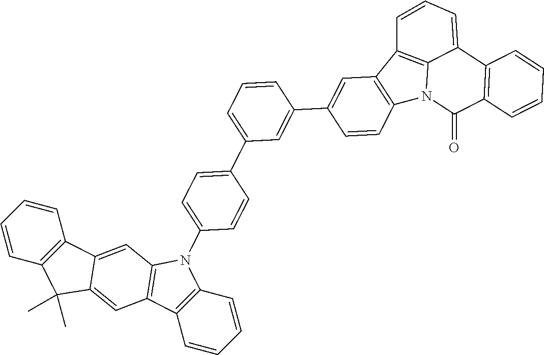 [SMILES: CC1(C)c2ccccc2-c2cc3c(cc21)c1ccccc1n3-c1ccc(-c2cccc(-c3ccc4c(c3)c3cccc5c6ccccc6c(=O)n4c53)c2)cc1]

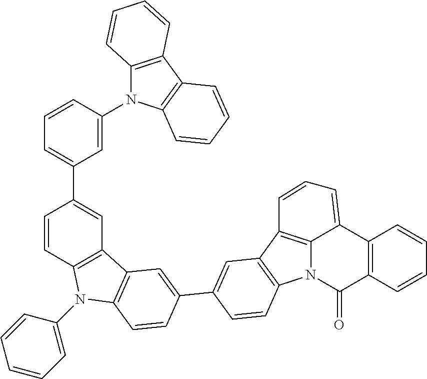 [SMILES: O=c1c2ccccc2c2cccc3c4cc(-c5ccc6c(c5)c5cc(-c7cccc(-n8c9ccccc9c9ccccc98)c7)ccc5n6-c5ccccc5)ccc4n1c23]

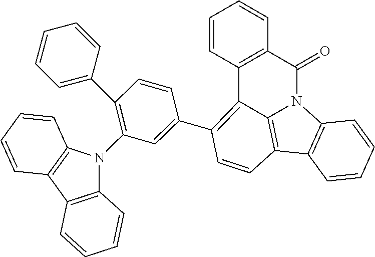 [SMILES: O=c1c2ccccc2c2c(-c3ccc(-c4ccccc4)c(-n4c5ccccc5c5ccccc54)c3)ccc3c4ccccc4n1c32]

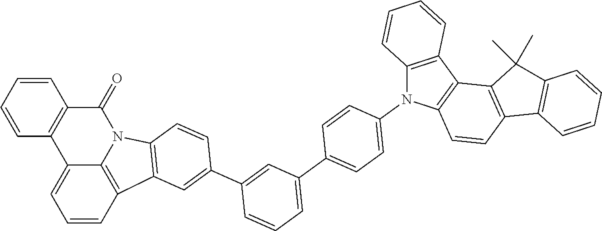 [SMILES: CC1(C)c2ccccc2-c2ccc3c(c21)c1ccccc1n3-c1ccc(-c2cccc(-c3ccc4c(c3)c3cccc5c6ccccc6c(=O)n4c53)c2)cc1]

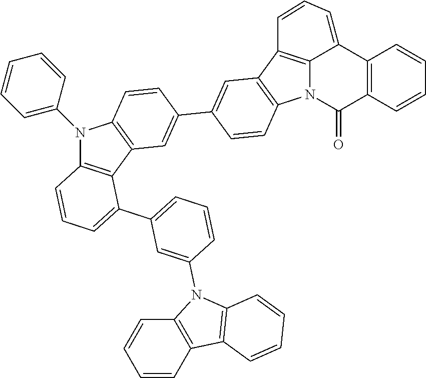 [SMILES: O=c1c2ccccc2c2cccc3c4cc(-c5ccc6c(c5)c5c(-c7cccc(-n8c9ccccc9c9ccccc98)c7)cccc5n6-c5ccccc5)ccc4n1c23]

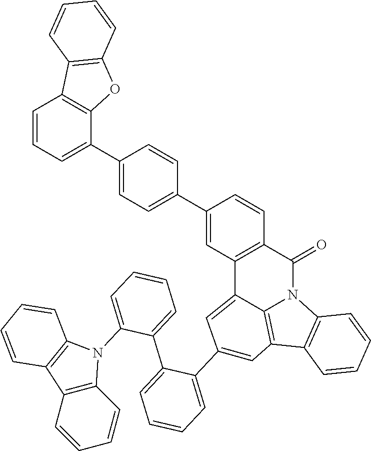 [SMILES: O=c1c2ccc(-c3ccc(-c4cccc5c4oc4ccccc45)cc3)cc2c2cc(-c3ccccc3-c3ccccc3-n3c4ccccc4c4ccccc43)cc3c4ccccc4n1c23]

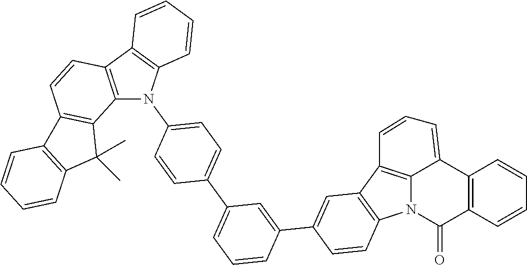 [SMILES: CC1(C)c2ccccc2-c2ccc3c4ccccc4n(-c4ccc(-c5cccc(-c6ccc7c(c6)c6cccc8c9ccccc9c(=O)n7c86)c5)cc4)c3c21]

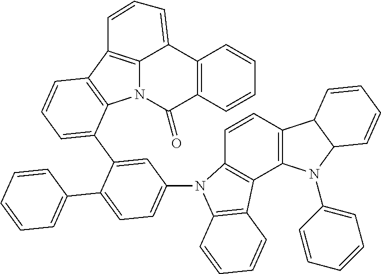 [SMILES: O=c1c2ccccc2c2cccc3c4cccc(-c5cc(-n6c7ccccc7c7c8c(ccc76)C6C=CC=CC6N8c6ccccc6)ccc5-c5ccccc5)c4n1c23]

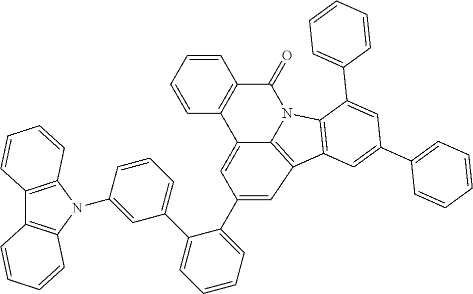 [SMILES: O=c1c2ccccc2c2cc(-c3ccccc3-c3cccc(-n4c5ccccc5c5ccccc54)c3)cc3c4cc(-c5ccccc5)cc(-c5ccccc5)c4n1c23]

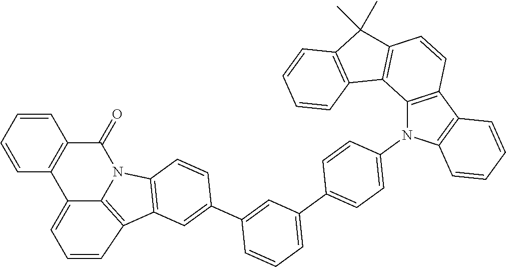 [SMILES: CC1(C)c2ccccc2-c2c1ccc1c3ccccc3n(-c3ccc(-c4cccc(-c5ccc6c(c5)c5cccc7c8ccccc8c(=O)n6c75)c4)cc3)c21]

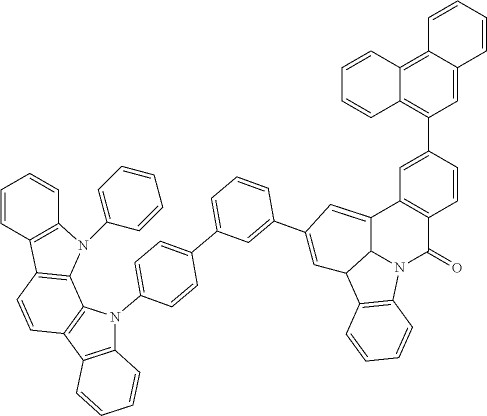 [SMILES: O=C1c2ccc(-c3cc4ccccc4c4ccccc34)cc2C2=CC(c3cccc(-c4ccc(-n5c6ccccc6c6ccc7c8ccccc8n(-c8ccccc8)c7c65)cc4)c3)=CC3c4ccccc4N1C23]

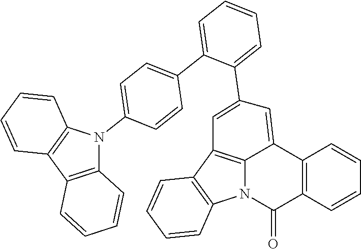 [SMILES: O=c1c2ccccc2c2cc(-c3ccccc3-c3ccc(-n4c5ccccc5c5ccccc54)cc3)cc3c4ccccc4n1c23]

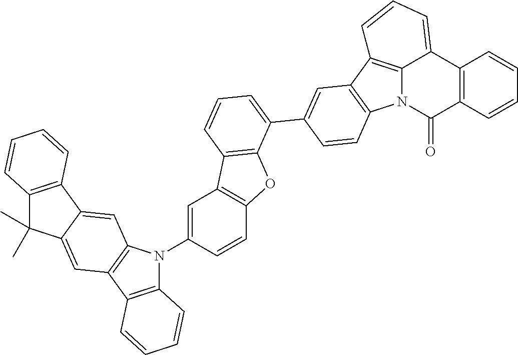 [SMILES: CC1(C)c2ccccc2-c2cc3c(cc21)c1ccccc1n3-c1ccc2oc3c(-c4ccc5c(c4)c4cccc6c7ccccc7c(=O)n5c64)cccc3c2c1]

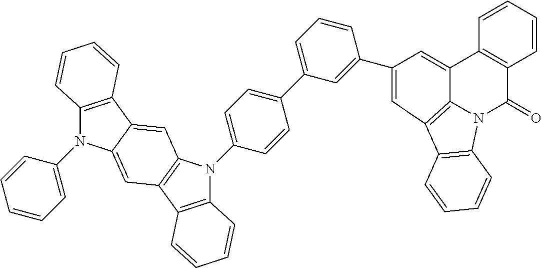 [SMILES: O=c1c2ccccc2c2cc(-c3cccc(-c4ccc(-n5c6ccccc6c6cc7c(cc65)c5ccccc5n7-c5ccccc5)cc4)c3)cc3c4ccccc4n1c23]

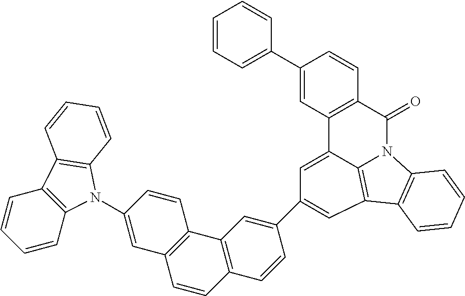 [SMILES: O=c1c2ccc(-c3ccccc3)cc2c2cc(-c3ccc4ccc5cc(-n6c7ccccc7c7ccccc76)ccc5c4c3)cc3c4ccccc4n1c23]

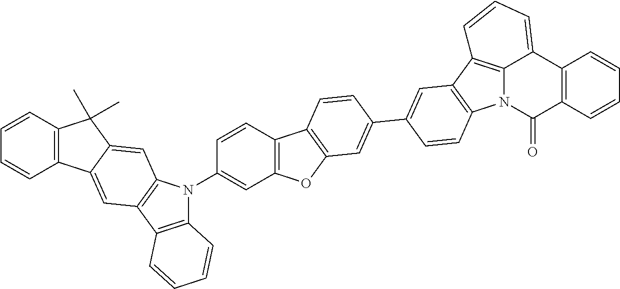 [SMILES: CC1(C)c2ccccc2-c2cc3c4ccccc4n(-c4ccc5c(c4)oc4cc(-c6ccc7c(c6)c6cccc8c9ccccc9c(=O)n7c86)ccc45)c3cc21]

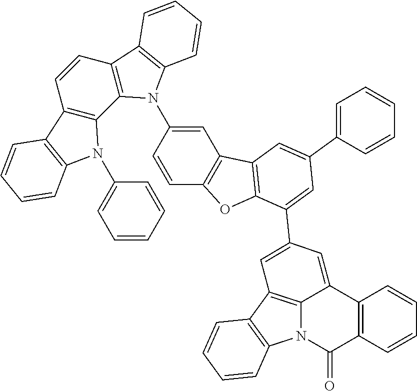 [SMILES: O=c1c2ccccc2c2cc(-c3cc(-c4ccccc4)cc4c3oc3ccc(-n5c6ccccc6c6ccc7c8ccccc8n(-c8ccccc8)c7c65)cc34)cc3c4ccccc4n1c23]

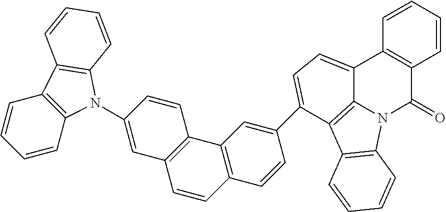 [SMILES: O=c1c2ccccc2c2ccc(-c3ccc4ccc5cc(-n6c7ccccc7c7ccccc76)ccc5c4c3)c3c4ccccc4n1c23]

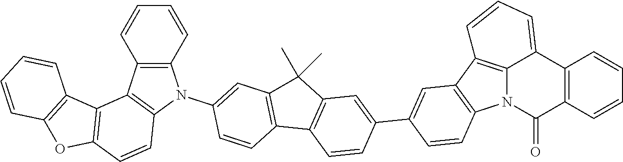 [SMILES: CC1(C)c2cc(-c3ccc4c(c3)c3cccc5c6ccccc6c(=O)n4c53)ccc2-c2ccc(-n3c4ccccc4c4c5c(ccc43)oc3ccccc35)cc21]

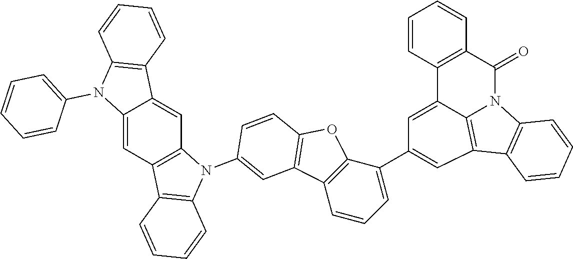 [SMILES: O=c1c2ccccc2c2cc(-c3cccc4c3oc3ccc(-n5c6ccccc6c6cc7c(cc65)c5ccccc5n7-c5ccccc5)cc34)cc3c4ccccc4n1c23]

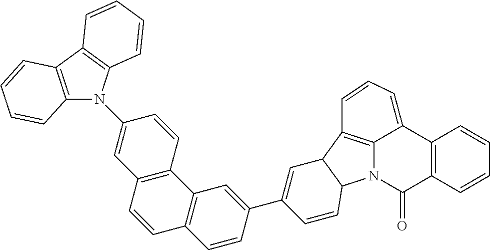 [SMILES: O=c1c2ccccc2c2cccc3c2n1C1C=CC(c2ccc4ccc5cc(-n6c7ccccc7c7ccccc76)ccc5c4c2)=CC31]

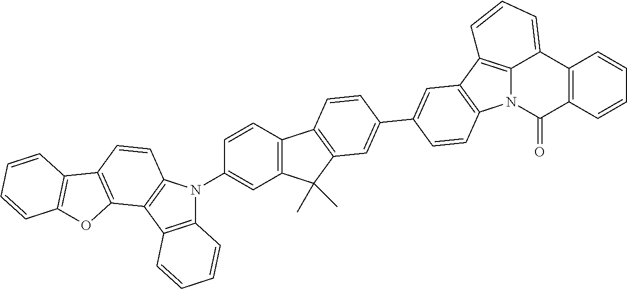 [SMILES: CC1(C)c2cc(-c3ccc4c(c3)c3cccc5c6ccccc6c(=O)n4c53)ccc2-c2ccc(-n3c4ccccc4c4c5oc6ccccc6c5ccc43)cc21]

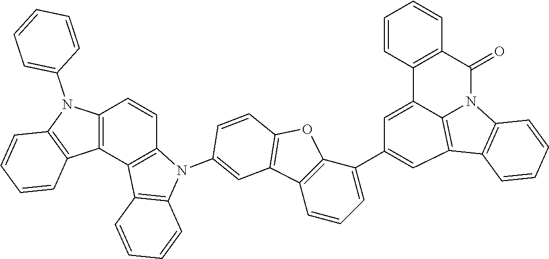 [SMILES: O=c1c2ccccc2c2cc(-c3cccc4c3oc3ccc(-n5c6ccccc6c6c7c8ccccc8n(-c8ccccc8)c7ccc65)cc34)cc3c4ccccc4n1c23]

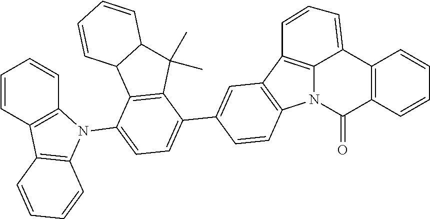 [SMILES: CC1(C)c2c(-c3ccc4c(c3)c3cccc5c6ccccc6c(=O)n4c53)ccc(-n3c4ccccc4c4ccccc43)c2C2C=CC=CC21]

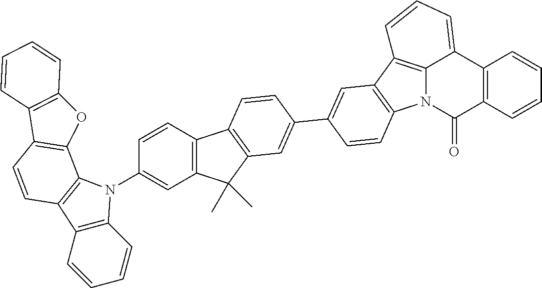 [SMILES: CC1(C)c2cc(-c3ccc4c(c3)c3cccc5c6ccccc6c(=O)n4c53)ccc2-c2ccc(-n3c4ccccc4c4ccc5c6ccccc6oc5c43)cc21]

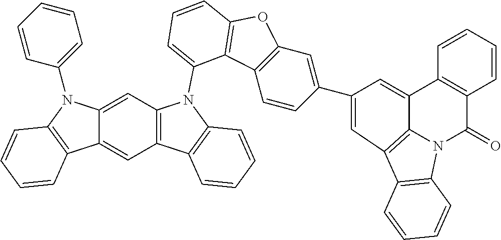 [SMILES: O=c1c2ccccc2c2cc(-c3ccc4c(c3)oc3cccc(-n5c6ccccc6c6cc7c8ccccc8n(-c8ccccc8)c7cc65)c34)cc3c4ccccc4n1c23]

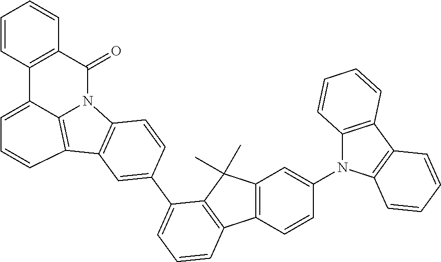 [SMILES: CC1(C)c2cc(-n3c4ccccc4c4ccccc43)ccc2-c2cccc(-c3ccc4c(c3)c3cccc5c6ccccc6c(=O)n4c53)c21]

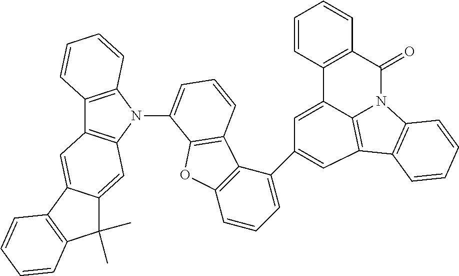 [SMILES: CC1(C)c2ccccc2-c2cc3c4ccccc4n(-c4cccc5c4oc4cccc(-c6cc7c8ccccc8c(=O)n8c9ccccc9c(c6)c78)c45)c3cc21]

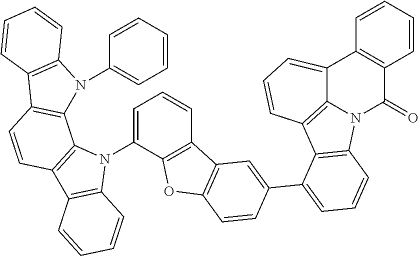 [SMILES: O=c1c2ccccc2c2cccc3c4c(-c5ccc6oc7c(-n8c9ccccc9c9ccc%10c%11ccccc%11n(-c%11ccccc%11)c%10c98)cccc7c6c5)cccc4n1c23]

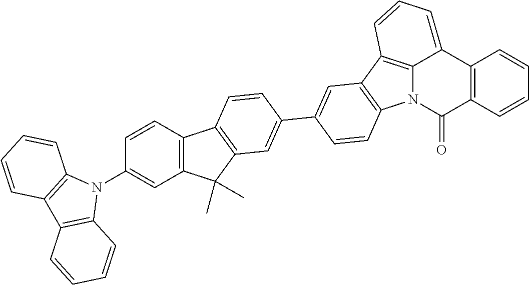 [SMILES: CC1(C)c2cc(-c3ccc4c(c3)c3cccc5c6ccccc6c(=O)n4c53)ccc2-c2ccc(-n3c4ccccc4c4ccccc43)cc21]

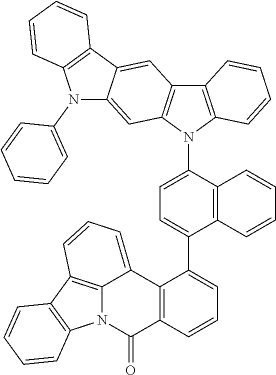 [SMILES: O=c1c2cccc(-c3ccc(-n4c5ccccc5c5cc6c7ccccc7n(-c7ccccc7)c6cc54)c4ccccc34)c2c2cccc3c4ccccc4n1c32]

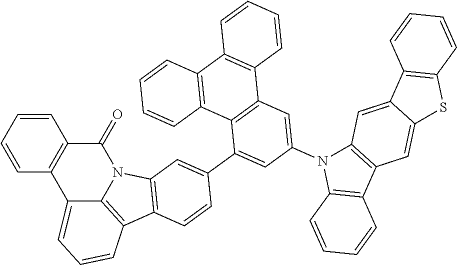 [SMILES: O=c1c2ccccc2c2cccc3c4ccc(-c5cc(-n6c7ccccc7c7cc8sc9ccccc9c8cc76)cc6c7ccccc7c7ccccc7c56)cc4n1c23]

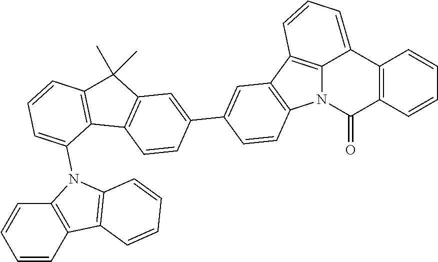 [SMILES: CC1(C)c2cc(-c3ccc4c(c3)c3cccc5c6ccccc6c(=O)n4c53)ccc2-c2c(-n3c4ccccc4c4ccccc43)cccc21]

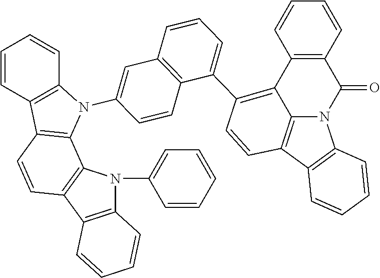 [SMILES: O=c1c2ccccc2c2c(-c3cccc4cc(-n5c6ccccc6c6ccc7c8ccccc8n(-c8ccccc8)c7c65)ccc34)ccc3c4ccccc4n1c32]

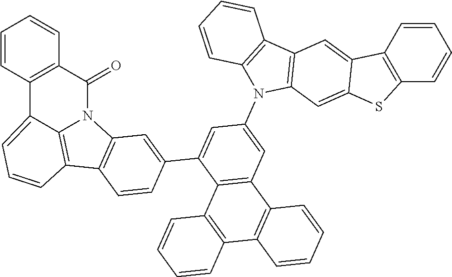 [SMILES: O=c1c2ccccc2c2cccc3c4ccc(-c5cc(-n6c7ccccc7c7cc8c(cc76)sc6ccccc68)cc6c7ccccc7c7ccccc7c56)cc4n1c23]

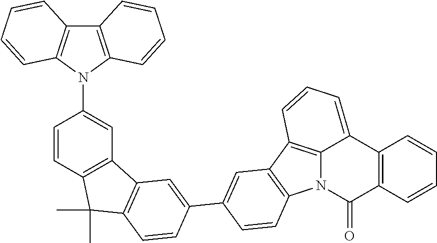 [SMILES: CC1(C)c2ccc(-c3ccc4c(c3)c3cccc5c6ccccc6c(=O)n4c53)cc2-c2cc(-n3c4ccccc4c4ccccc43)ccc21]

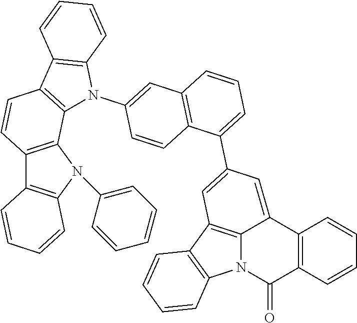 [SMILES: O=c1c2ccccc2c2cc(-c3cccc4cc(-n5c6ccccc6c6ccc7c8ccccc8n(-c8ccccc8)c7c65)ccc34)cc3c4ccccc4n1c23]

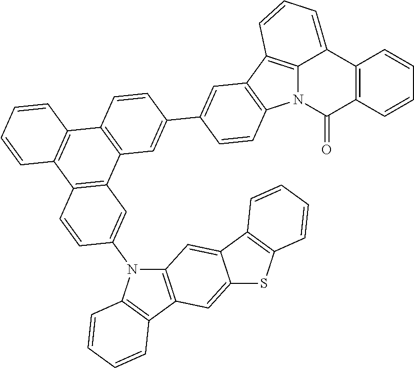 [SMILES: O=c1c2ccccc2c2cccc3c4cc(-c5ccc6c7ccccc7c7ccc(-n8c9ccccc9c9cc%10sc%11ccccc%11c%10cc98)cc7c6c5)ccc4n1c23]

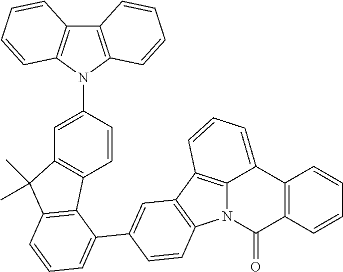 [SMILES: CC1(C)c2cc(-n3c4ccccc4c4ccccc43)ccc2-c2c(-c3ccc4c(c3)c3cccc5c6ccccc6c(=O)n4c53)cccc21]

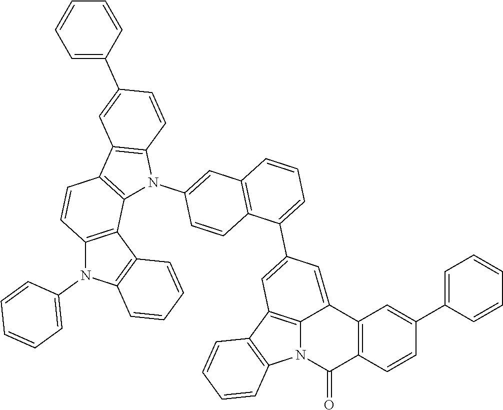 [SMILES: O=c1c2ccc(-c3ccccc3)cc2c2cc(-c3cccc4cc(-n5c6ccc(-c7ccccc7)cc6c6ccc7c(c8ccccc8n7-c7ccccc7)c65)ccc34)cc3c4ccccc4n1c23]

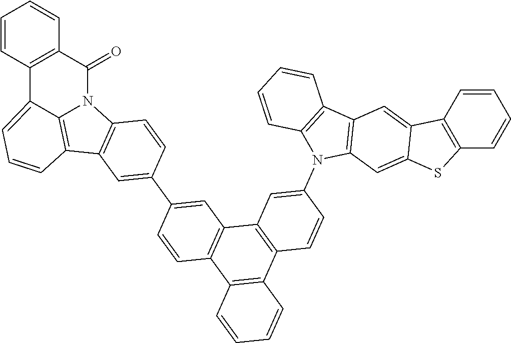 [SMILES: O=c1c2ccccc2c2cccc3c4cc(-c5ccc6c7ccccc7c7ccc(-n8c9ccccc9c9cc%10c(cc98)sc8ccccc8%10)cc7c6c5)ccc4n1c23]

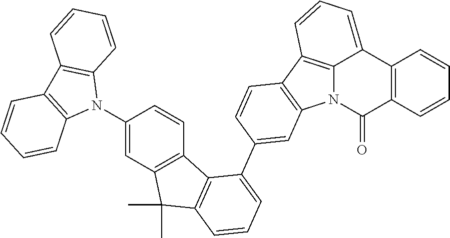 [SMILES: CC1(C)c2cc(-n3c4ccccc4c4ccccc43)ccc2-c2c(-c3ccc4c5cccc6c7ccccc7c(=O)n(c4c3)c65)cccc21]

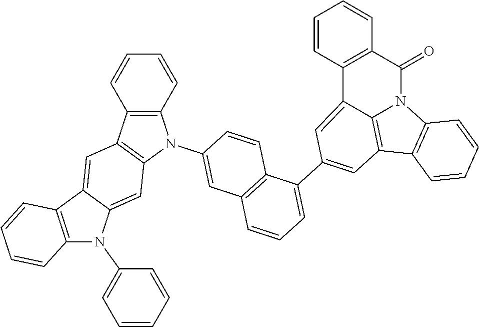 [SMILES: O=c1c2ccccc2c2cc(-c3cccc4cc(-n5c6ccccc6c6cc7c8ccccc8n(-c8ccccc8)c7cc65)ccc34)cc3c4ccccc4n1c23]

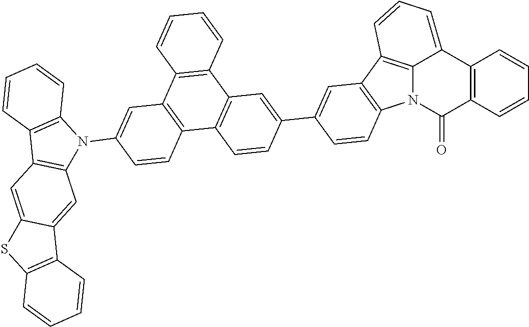 [SMILES: O=c1c2ccccc2c2cccc3c4cc(-c5ccc6c7ccc(-n8c9ccccc9c9cc%10sc%11ccccc%11c%10cc98)cc7c7ccccc7c6c5)ccc4n1c23]

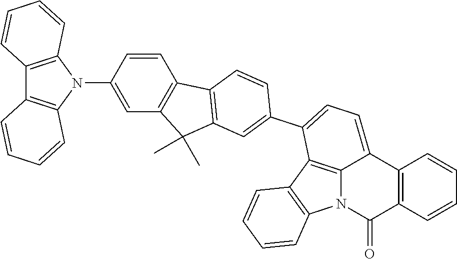 [SMILES: CC1(C)c2cc(-c3ccc4c5ccccc5c(=O)n5c6ccccc6c3c45)ccc2-c2ccc(-n3c4ccccc4c4ccccc43)cc21]

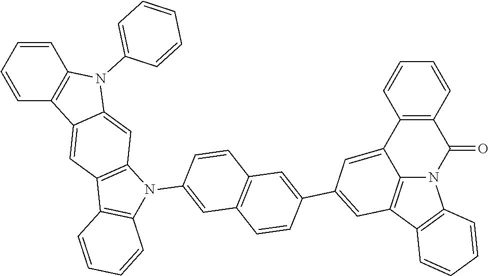 [SMILES: O=c1c2ccccc2c2cc(-c3ccc4cc(-n5c6ccccc6c6cc7c8ccccc8n(-c8ccccc8)c7cc65)ccc4c3)cc3c4ccccc4n1c23]

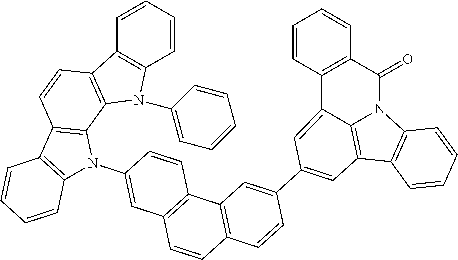 [SMILES: O=c1c2ccccc2c2cc(-c3ccc4ccc5cc(-n6c7ccccc7c7ccc8c9ccccc9n(-c9ccccc9)c8c76)ccc5c4c3)cc3c4ccccc4n1c23]

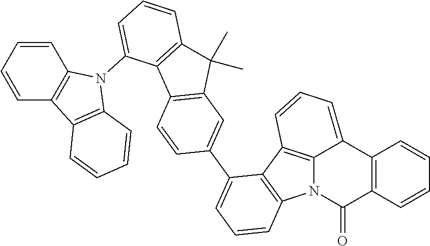 [SMILES: CC1(C)c2cc(-c3cccc4c3c3cccc5c6ccccc6c(=O)n4c53)ccc2-c2c(-n3c4ccccc4c4ccccc43)cccc21]

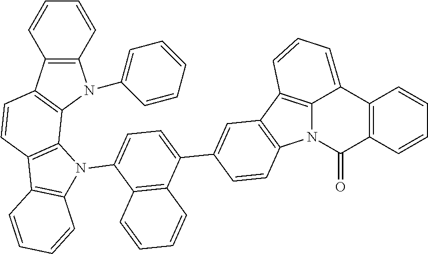 [SMILES: O=c1c2ccccc2c2cccc3c4cc(-c5ccc(-n6c7ccccc7c7ccc8c9ccccc9n(-c9ccccc9)c8c76)c6ccccc56)ccc4n1c23]

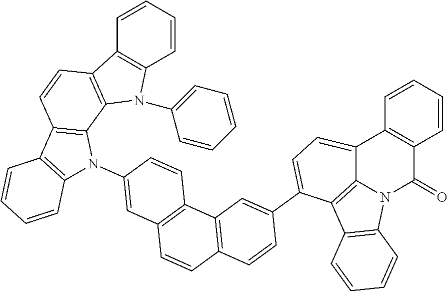 [SMILES: O=c1c2ccccc2c2ccc(-c3ccc4ccc5cc(-n6c7ccccc7c7ccc8c9ccccc9n(-c9ccccc9)c8c76)ccc5c4c3)c3c4ccccc4n1c23]

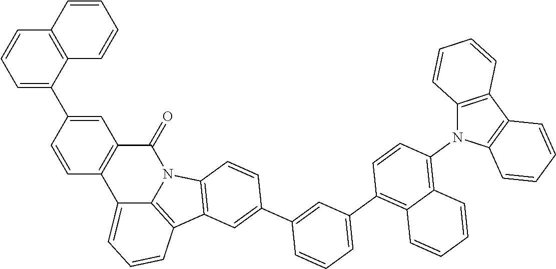 [SMILES: O=c1c2cc(-c3cccc4ccccc34)ccc2c2cccc3c4cc(-c5cccc(-c6ccc(-n7c8ccccc8c8ccccc87)c7ccccc67)c5)ccc4n1c23]

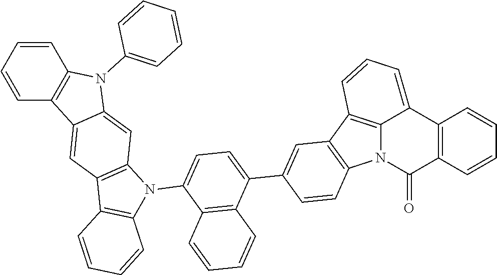 [SMILES: O=c1c2ccccc2c2cccc3c4cc(-c5ccc(-n6c7ccccc7c7cc8c9ccccc9n(-c9ccccc9)c8cc76)c6ccccc56)ccc4n1c23]

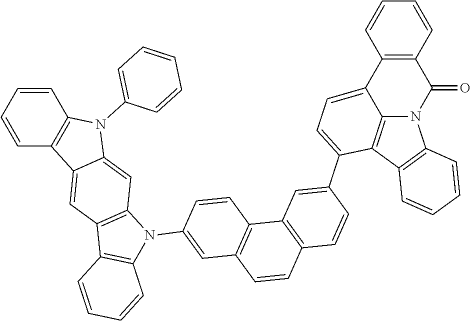 [SMILES: O=c1c2ccccc2c2ccc(-c3ccc4ccc5cc(-n6c7ccccc7c7cc8c9ccccc9n(-c9ccccc9)c8cc76)ccc5c4c3)c3c4ccccc4n1c23]

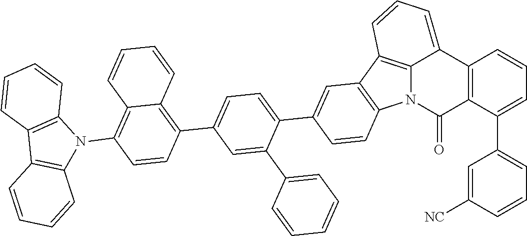 [SMILES: N#Cc1cccc(-c2cccc3c2c(=O)n2c4ccc(-c5ccc(-c6ccc(-n7c8ccccc8c8ccccc87)c7ccccc67)cc5-c5ccccc5)cc4c4cccc3c42)c1]

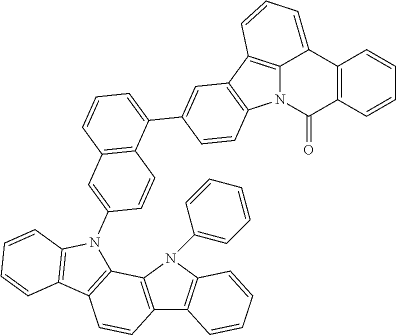 [SMILES: O=c1c2ccccc2c2cccc3c4cc(-c5cccc6cc(-n7c8ccccc8c8ccc9c%10ccccc%10n(-c%10ccccc%10)c9c87)ccc56)ccc4n1c23]

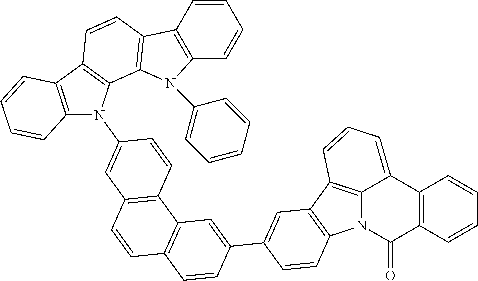 [SMILES: O=c1c2ccccc2c2cccc3c4cc(-c5ccc6ccc7cc(-n8c9ccccc9c9ccc%10c%11ccccc%11n(-c%11ccccc%11)c%10c98)ccc7c6c5)ccc4n1c23]

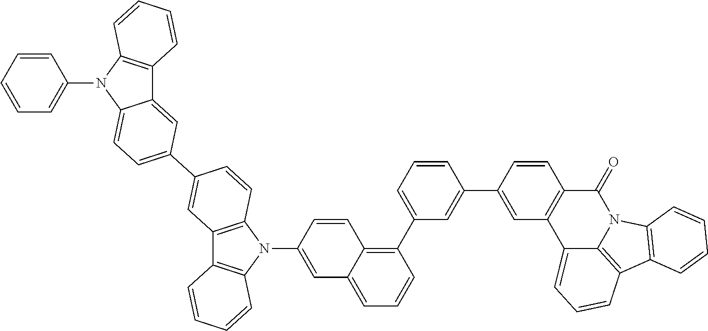 [SMILES: O=c1c2ccc(-c3cccc(-c4cccc5cc(-n6c7ccccc7c7cc(-c8ccc9c(c8)c8ccccc8n9-c8ccccc8)ccc76)ccc45)c3)cc2c2cccc3c4ccccc4n1c23]

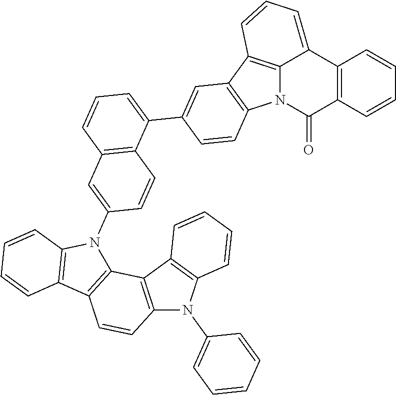 [SMILES: O=c1c2ccccc2c2cccc3c4cc(-c5cccc6cc(-n7c8ccccc8c8ccc9c(c%10ccccc%10n9-c9ccccc9)c87)ccc56)ccc4n1c23]

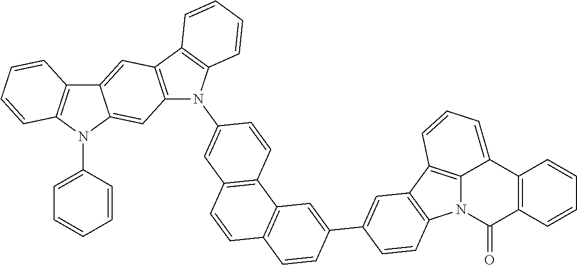 [SMILES: O=c1c2ccccc2c2cccc3c4cc(-c5ccc6ccc7cc(-n8c9ccccc9c9cc%10c%11ccccc%11n(-c%11ccccc%11)c%10cc98)ccc7c6c5)ccc4n1c23]

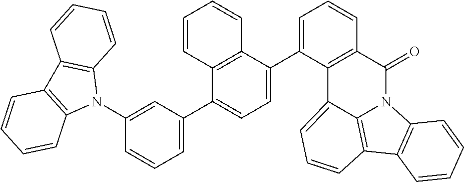 [SMILES: O=c1c2cccc(-c3ccc(-c4cccc(-n5c6ccccc6c6ccccc65)c4)c4ccccc34)c2c2cccc3c4ccccc4n1c32]

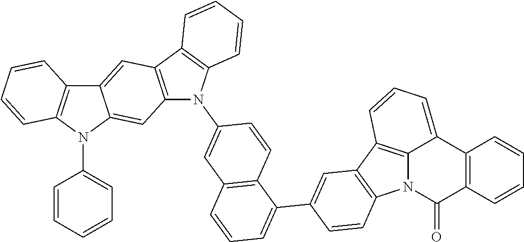 [SMILES: O=c1c2ccccc2c2cccc3c4cc(-c5cccc6cc(-n7c8ccccc8c8cc9c%10ccccc%10n(-c%10ccccc%10)c9cc87)ccc56)ccc4n1c23]

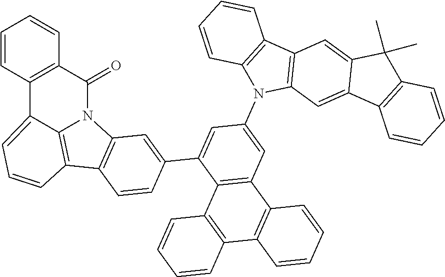 [SMILES: CC1(C)c2ccccc2-c2cc3c(cc21)c1ccccc1n3-c1cc(-c2ccc3c4cccc5c6ccccc6c(=O)n(c3c2)c54)c2c3ccccc3c3ccccc3c2c1]

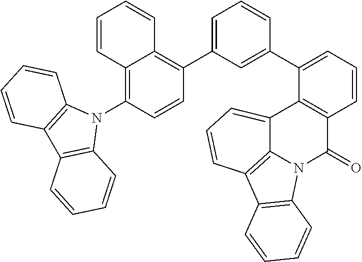 [SMILES: O=c1c2cccc(-c3cccc(-c4ccc(-n5c6ccccc6c6ccccc65)c5ccccc45)c3)c2c2cccc3c4ccccc4n1c32]

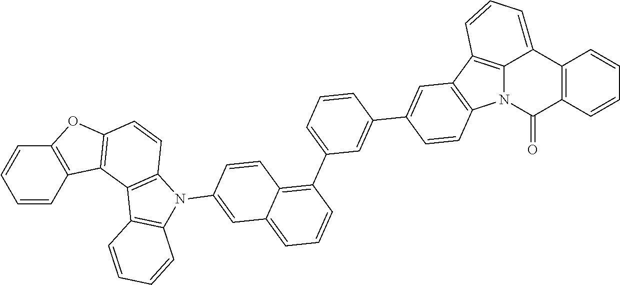 [SMILES: O=c1c2ccccc2c2cccc3c4cc(-c5cccc(-c6cccc7cc(-n8c9ccccc9c9c%10c(ccc98)oc8ccccc8%10)ccc67)c5)ccc4n1c23]

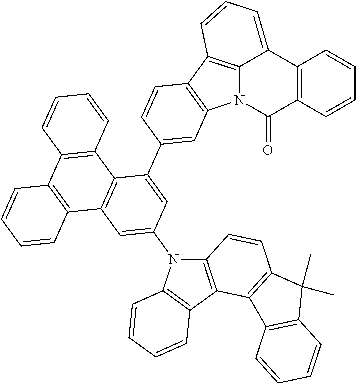 [SMILES: CC1(C)c2ccccc2-c2c1ccc1c2c2ccccc2n1-c1cc(-c2ccc3c4cccc5c6ccccc6c(=O)n(c3c2)c54)c2c3ccccc3c3ccccc3c2c1]

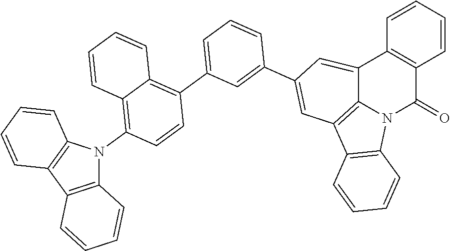 [SMILES: O=c1c2ccccc2c2cc(-c3cccc(-c4ccc(-n5c6ccccc6c6ccccc65)c5ccccc45)c3)cc3c4ccccc4n1c23]

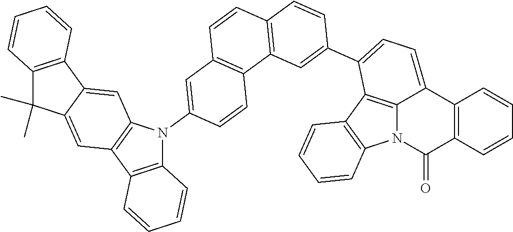 [SMILES: CC1(C)c2ccccc2-c2cc3c(cc21)c1ccccc1n3-c1ccc2c(ccc3ccc(-c4ccc5c6ccccc6c(=O)n6c7ccccc7c4c56)cc32)c1]

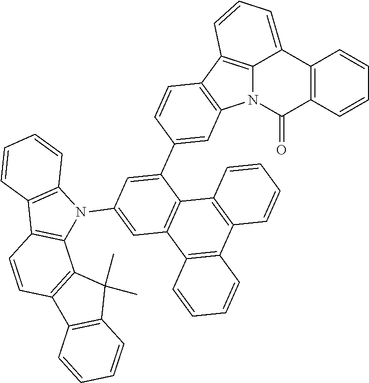 [SMILES: CC1(C)c2ccccc2-c2ccc3c4ccccc4n(-c4cc(-c5ccc6c7cccc8c9ccccc9c(=O)n(c6c5)c87)c5c6ccccc6c6ccccc6c5c4)c3c21]

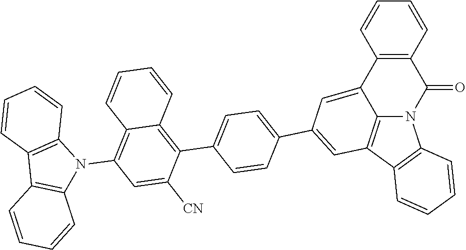 [SMILES: N#Cc1cc(-n2c3ccccc3c3ccccc32)c2ccccc2c1-c1ccc(-c2cc3c4ccccc4c(=O)n4c5ccccc5c(c2)c34)cc1]

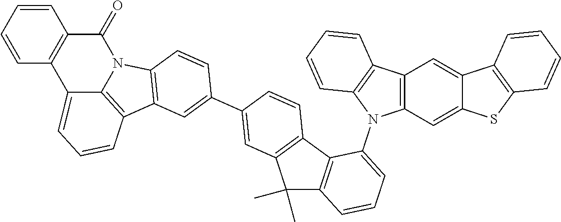 [SMILES: CC1(C)c2cc(-c3ccc4c(c3)c3cccc5c6ccccc6c(=O)n4c53)ccc2-c2c(-n3c4ccccc4c4cc5c(cc43)sc3ccccc35)cccc21]

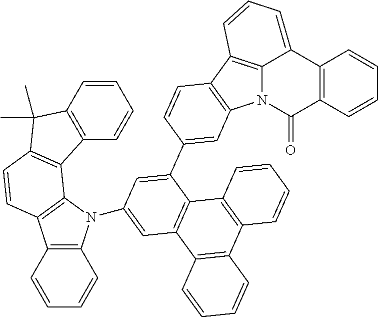 [SMILES: CC1(C)c2ccccc2-c2c1ccc1c3ccccc3n(-c3cc(-c4ccc5c6cccc7c8ccccc8c(=O)n(c5c4)c76)c4c5ccccc5c5ccccc5c4c3)c21]

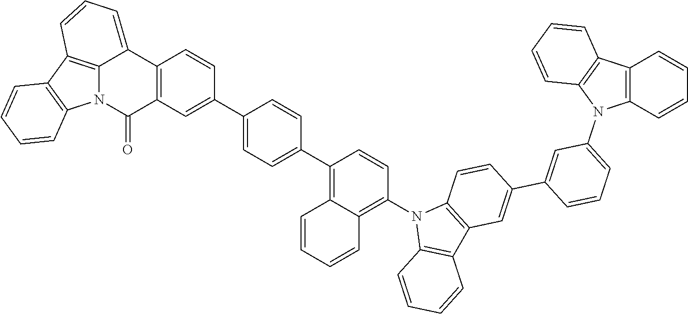 [SMILES: O=c1c2cc(-c3ccc(-c4ccc(-n5c6ccccc6c6cc(-c7cccc(-n8c9ccccc9c9ccccc98)c7)ccc65)c5ccccc45)cc3)ccc2c2cccc3c4ccccc4n1c23]

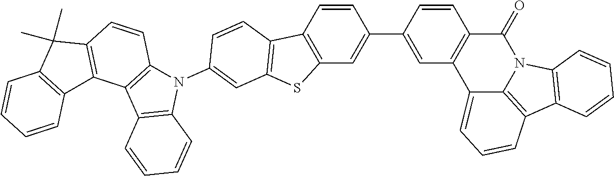 [SMILES: CC1(C)c2ccccc2-c2c1ccc1c2c2ccccc2n1-c1ccc2c(c1)sc1cc(-c3ccc4c(=O)n5c6ccccc6c6cccc(c4c3)c65)ccc12]

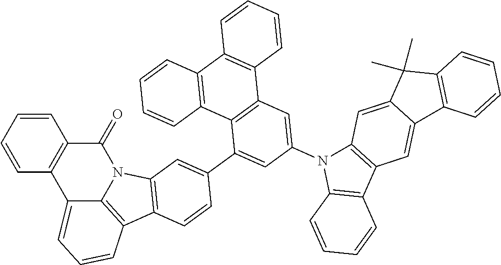 [SMILES: CC1(C)c2ccccc2-c2cc3c4ccccc4n(-c4cc(-c5ccc6c7cccc8c9ccccc9c(=O)n(c6c5)c87)c5c6ccccc6c6ccccc6c5c4)c3cc21]

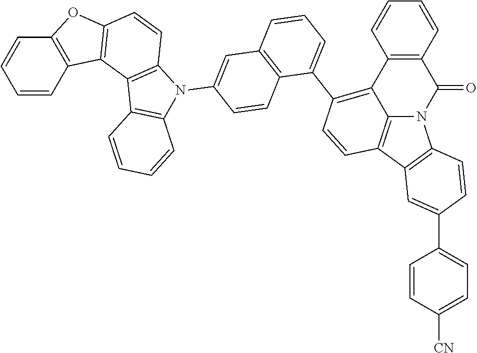 [SMILES: N#Cc1ccc(-c2ccc3c(c2)c2ccc(-c4cccc5cc(-n6c7ccccc7c7c8c(ccc76)oc6ccccc68)ccc45)c4c5ccccc5c(=O)n3c24)cc1]

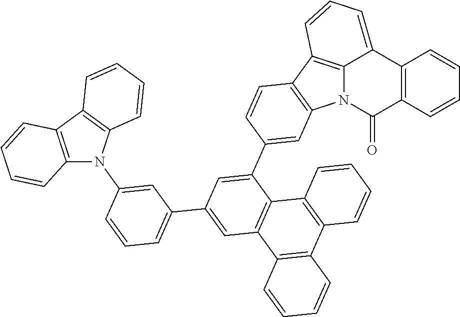 [SMILES: O=c1c2ccccc2c2cccc3c4ccc(-c5cc(-c6cccc(-n7c8ccccc8c8ccccc87)c6)cc6c7ccccc7c7ccccc7c56)cc4n1c23]

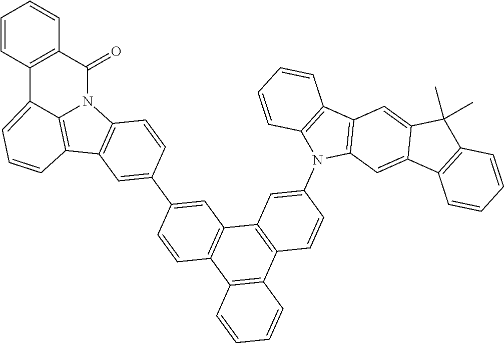 [SMILES: CC1(C)c2ccccc2-c2cc3c(cc21)c1ccccc1n3-c1ccc2c3ccccc3c3ccc(-c4ccc5c(c4)c4cccc6c7ccccc7c(=O)n5c64)cc3c2c1]

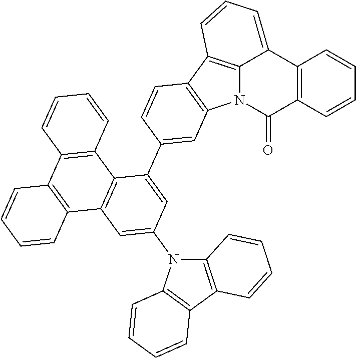 [SMILES: O=c1c2ccccc2c2cccc3c4ccc(-c5cc(-n6c7ccccc7c7ccccc76)cc6c7ccccc7c7ccccc7c56)cc4n1c23]

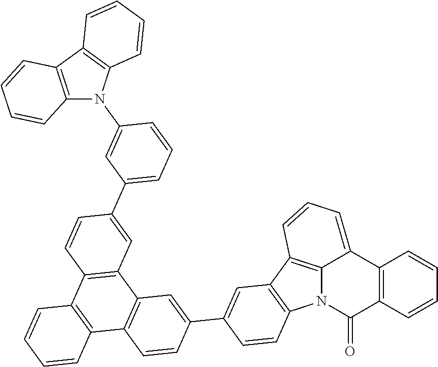 [SMILES: O=c1c2ccccc2c2cccc3c4cc(-c5ccc6c7ccccc7c7ccc(-c8cccc(-n9c%10ccccc%10c%10ccccc%109)c8)cc7c6c5)ccc4n1c23]

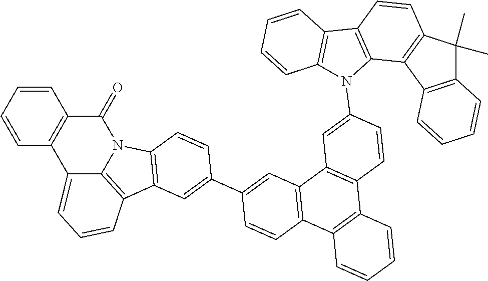 [SMILES: CC1(C)c2ccccc2-c2c1ccc1c3ccccc3n(-c3ccc4c5ccccc5c5ccc(-c6ccc7c(c6)c6cccc8c9ccccc9c(=O)n7c86)cc5c4c3)c21]

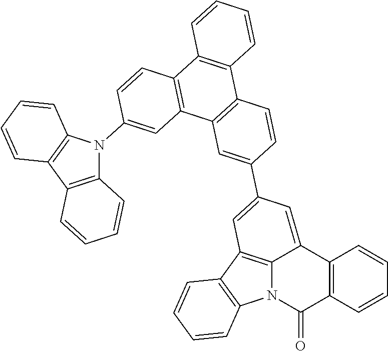 [SMILES: O=c1c2ccccc2c2cc(-c3ccc4c5ccccc5c5ccc(-n6c7ccccc7c7ccccc76)cc5c4c3)cc3c4ccccc4n1c23]

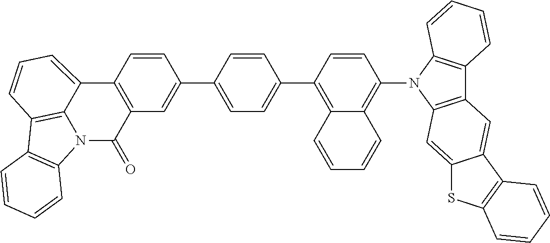 [SMILES: O=c1c2cc(-c3ccc(-c4ccc(-n5c6ccccc6c6cc7c(cc65)sc5ccccc57)c5ccccc45)cc3)ccc2c2cccc3c4ccccc4n1c23]

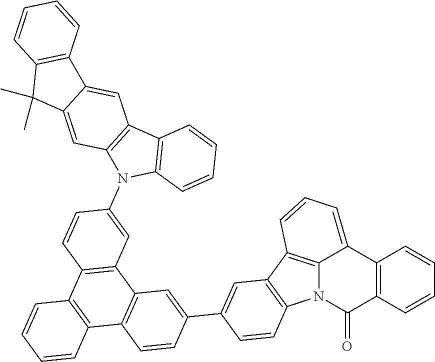 [SMILES: CC1(C)c2ccccc2-c2cc3c4ccccc4n(-c4ccc5c6ccccc6c6ccc(-c7ccc8c(c7)c7cccc9c%10ccccc%10c(=O)n8c97)cc6c5c4)c3cc21]

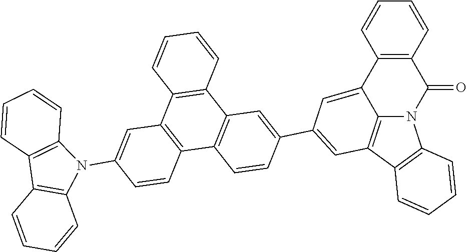 [SMILES: O=c1c2ccccc2c2cc(-c3ccc4c5ccc(-n6c7ccccc7c7ccccc76)cc5c5ccccc5c4c3)cc3c4ccccc4n1c23]

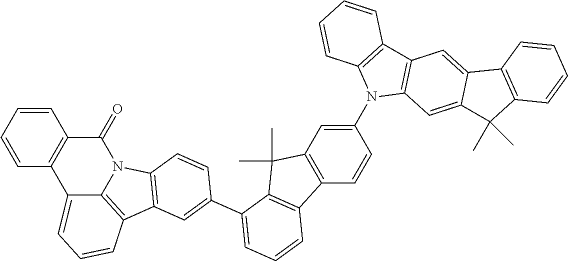 [SMILES: CC1(C)c2ccccc2-c2cc3c4ccccc4n(-c4ccc5c(c4)C(C)(C)c4c(-c6ccc7c(c6)c6cccc8c9ccccc9c(=O)n7c86)cccc4-5)c3cc21]

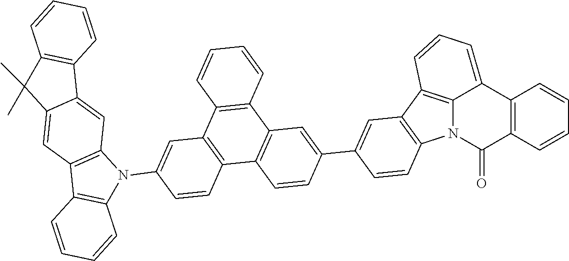 [SMILES: CC1(C)c2ccccc2-c2cc3c(cc21)c1ccccc1n3-c1ccc2c3ccc(-c4ccc5c(c4)c4cccc6c7ccccc7c(=O)n5c64)cc3c3ccccc3c2c1]

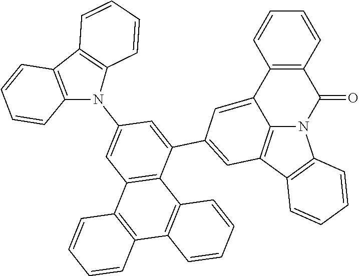 [SMILES: O=c1c2ccccc2c2cc(-c3cc(-n4c5ccccc5c5ccccc54)cc4c5ccccc5c5ccccc5c34)cc3c4ccccc4n1c23]

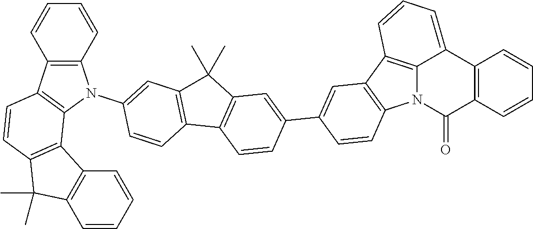 [SMILES: CC1(C)c2cc(-c3ccc4c(c3)c3cccc5c6ccccc6c(=O)n4c53)ccc2-c2ccc(-n3c4ccccc4c4ccc5c(c43)-c3ccccc3C5(C)C)cc21]

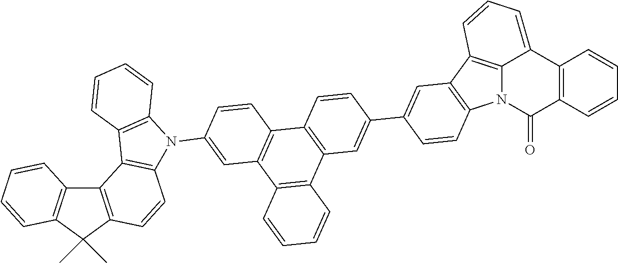 [SMILES: CC1(C)c2ccccc2-c2c1ccc1c2c2ccccc2n1-c1ccc2c3ccc(-c4ccc5c(c4)c4cccc6c7ccccc7c(=O)n5c64)cc3c3ccccc3c2c1]

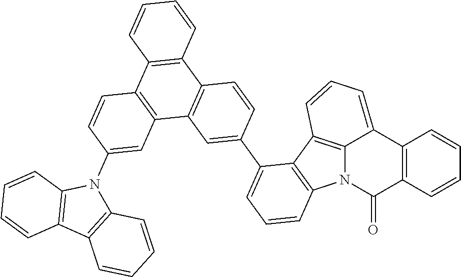 [SMILES: O=c1c2ccccc2c2cccc3c4c(-c5ccc6c7ccccc7c7ccc(-n8c9ccccc9c9ccccc98)cc7c6c5)cccc4n1c23]

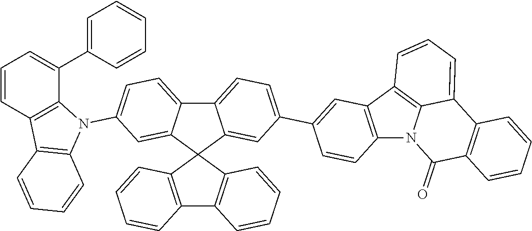 [SMILES: O=c1c2ccccc2c2cccc3c4cc(-c5ccc6c(c5)C5(c7ccccc7-c7ccccc75)c5cc(-n7c8ccccc8c8cccc(-c9ccccc9)c87)ccc5-6)ccc4n1c23]

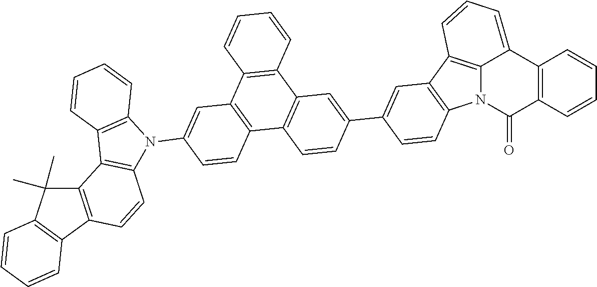 [SMILES: CC1(C)c2ccccc2-c2ccc3c(c21)c1ccccc1n3-c1ccc2c3ccc(-c4ccc5c(c4)c4cccc6c7ccccc7c(=O)n5c64)cc3c3ccccc3c2c1]

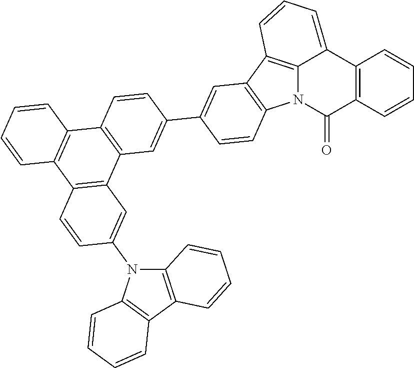 [SMILES: O=c1c2ccccc2c2cccc3c4cc(-c5ccc6c7ccccc7c7ccc(-n8c9ccccc9c9ccccc98)cc7c6c5)ccc4n1c23]

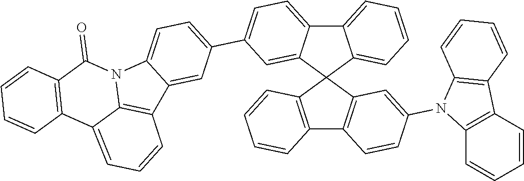 [SMILES: O=c1c2ccccc2c2cccc3c4cc(-c5ccc6c(c5)C5(c7ccccc7-6)c6ccccc6-c6ccc(-n7c8ccccc8c8ccccc87)cc65)ccc4n1c23]

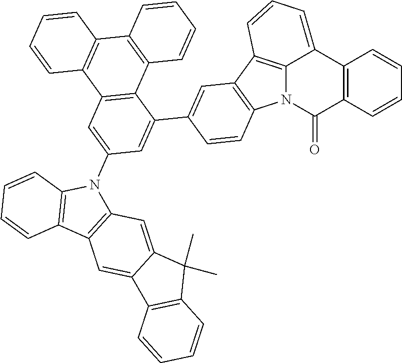 [SMILES: CC1(C)c2ccccc2-c2cc3c4ccccc4n(-c4cc(-c5ccc6c(c5)c5cccc7c8ccccc8c(=O)n6c75)c5c6ccccc6c6ccccc6c5c4)c3cc21]

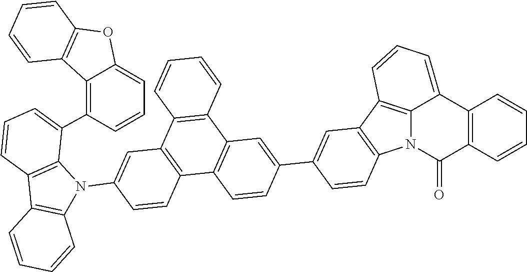 [SMILES: O=c1c2ccccc2c2cccc3c4cc(-c5ccc6c7ccc(-n8c9ccccc9c9cccc(-c%10cccc%11oc%12ccccc%12c%10%11)c98)cc7c7ccccc7c6c5)ccc4n1c23]

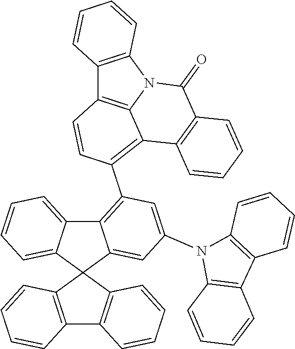 [SMILES: O=c1c2ccccc2c2c(-c3cc(-n4c5ccccc5c5ccccc54)cc4c3-c3ccccc3C43c4ccccc4-c4ccccc43)ccc3c4ccccc4n1c32]

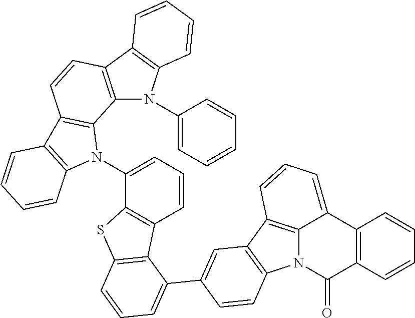 [SMILES: O=c1c2ccccc2c2cccc3c4cc(-c5cccc6sc7c(-n8c9ccccc9c9ccc%10c%11ccccc%11n(-c%11ccccc%11)c%10c98)cccc7c56)ccc4n1c23]

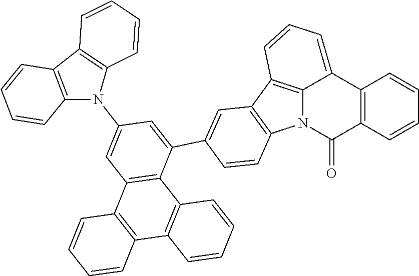 [SMILES: O=c1c2ccccc2c2cccc3c4cc(-c5cc(-n6c7ccccc7c7ccccc76)cc6c7ccccc7c7ccccc7c56)ccc4n1c23]

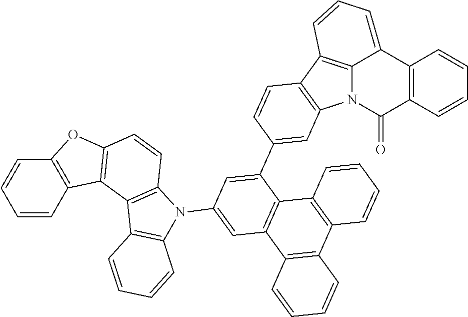 [SMILES: O=c1c2ccccc2c2cccc3c4ccc(-c5cc(-n6c7ccccc7c7c8c(ccc76)oc6ccccc68)cc6c7ccccc7c7ccccc7c56)cc4n1c23]

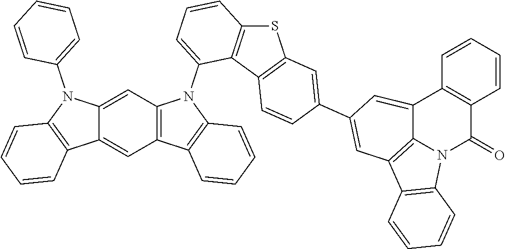 [SMILES: O=c1c2ccccc2c2cc(-c3ccc4c(c3)sc3cccc(-n5c6ccccc6c6cc7c8ccccc8n(-c8ccccc8)c7cc65)c34)cc3c4ccccc4n1c23]

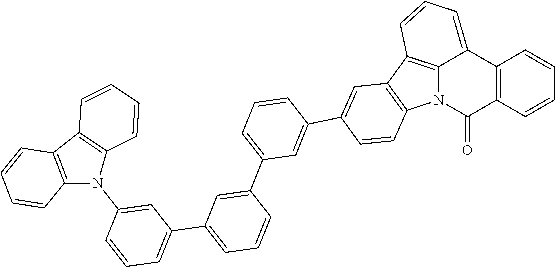 [SMILES: O=c1c2ccccc2c2cccc3c4cc(-c5cccc(-c6cccc(-c7cccc(-n8c9ccccc9c9ccccc98)c7)c6)c5)ccc4n1c23]

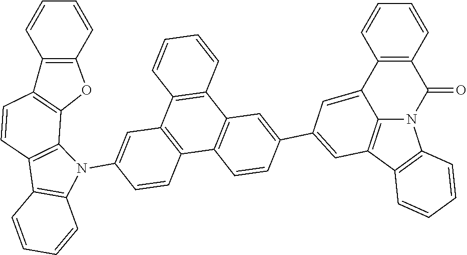 [SMILES: O=c1c2ccccc2c2cc(-c3ccc4c5ccc(-n6c7ccccc7c7ccc8c9ccccc9oc8c76)cc5c5ccccc5c4c3)cc3c4ccccc4n1c23]

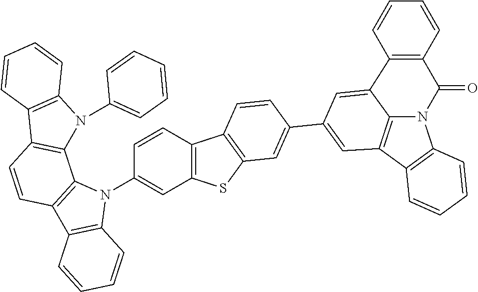 [SMILES: O=c1c2ccccc2c2cc(-c3ccc4c(c3)sc3cc(-n5c6ccccc6c6ccc7c8ccccc8n(-c8ccccc8)c7c65)ccc34)cc3c4ccccc4n1c23]

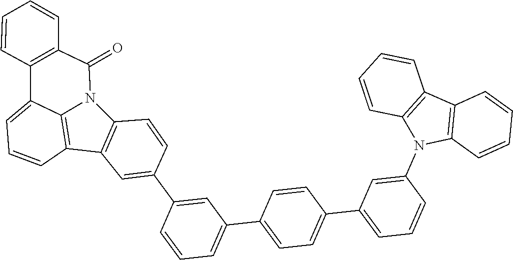 [SMILES: O=c1c2ccccc2c2cccc3c4cc(-c5cccc(-c6ccc(-c7cccc(-n8c9ccccc9c9ccccc98)c7)cc6)c5)ccc4n1c23]

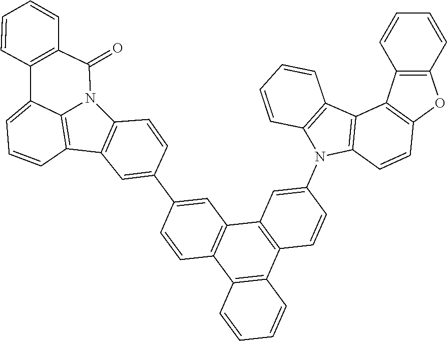 [SMILES: O=c1c2ccccc2c2cccc3c4cc(-c5ccc6c7ccccc7c7ccc(-n8c9ccccc9c9c%10c(ccc98)oc8ccccc8%10)cc7c6c5)ccc4n1c23]

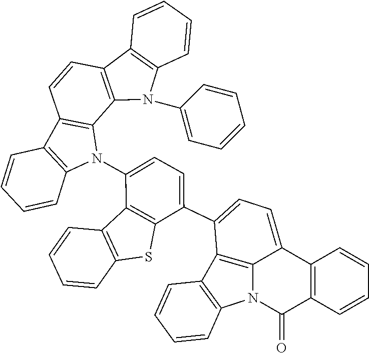 [SMILES: O=c1c2ccccc2c2ccc(-c3ccc(-n4c5ccccc5c5ccc6c7ccccc7n(-c7ccccc7)c6c54)c4c3sc3ccccc34)c3c4ccccc4n1c23]

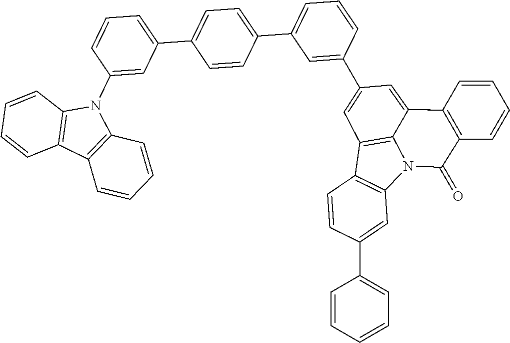 [SMILES: O=c1c2ccccc2c2cc(-c3cccc(-c4ccc(-c5cccc(-n6c7ccccc7c7ccccc76)c5)cc4)c3)cc3c4ccc(-c5ccccc5)cc4n1c23]

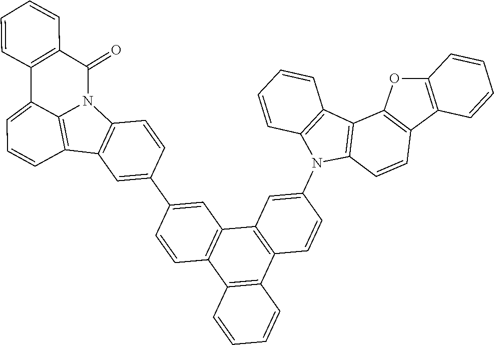 [SMILES: O=c1c2ccccc2c2cccc3c4cc(-c5ccc6c7ccccc7c7ccc(-n8c9ccccc9c9c%10oc%11ccccc%11c%10ccc98)cc7c6c5)ccc4n1c23]

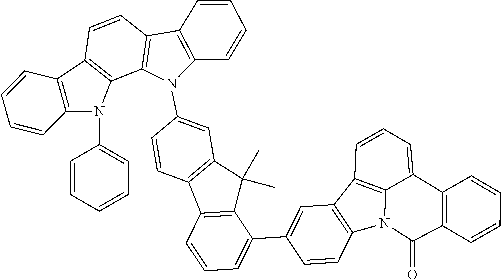 [SMILES: CC1(C)c2cc(-n3c4ccccc4c4ccc5c6ccccc6n(-c6ccccc6)c5c43)ccc2-c2cccc(-c3ccc4c(c3)c3cccc5c6ccccc6c(=O)n4c53)c21]

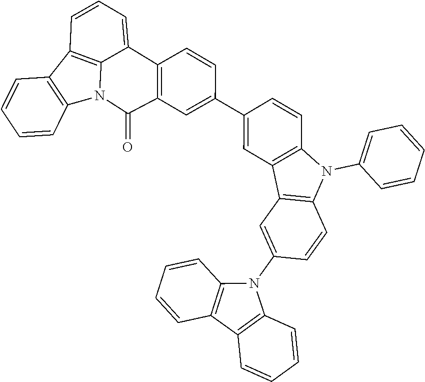 [SMILES: O=c1c2cc(-c3ccc4c(c3)c3cc(-n5c6ccccc6c6ccccc65)ccc3n4-c3ccccc3)ccc2c2cccc3c4ccccc4n1c23]

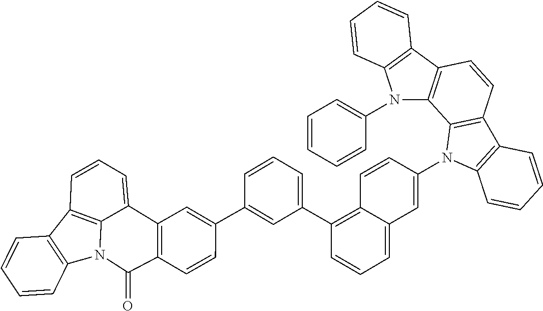 [SMILES: O=c1c2ccc(-c3cccc(-c4cccc5cc(-n6c7ccccc7c7ccc8c9ccccc9n(-c9ccccc9)c8c76)ccc45)c3)cc2c2cccc3c4ccccc4n1c23]

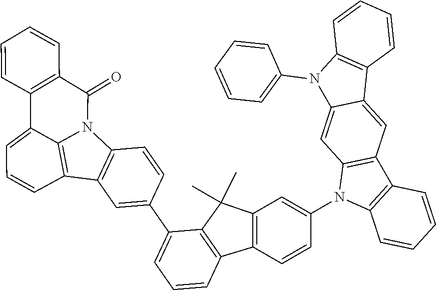 [SMILES: CC1(C)c2cc(-n3c4ccccc4c4cc5c6ccccc6n(-c6ccccc6)c5cc43)ccc2-c2cccc(-c3ccc4c(c3)c3cccc5c6ccccc6c(=O)n4c53)c21]

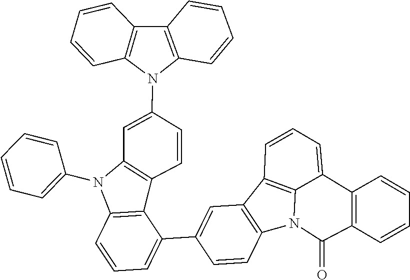 [SMILES: O=c1c2ccccc2c2cccc3c4cc(-c5cccc6c5c5ccc(-n7c8ccccc8c8ccccc87)cc5n6-c5ccccc5)ccc4n1c23]

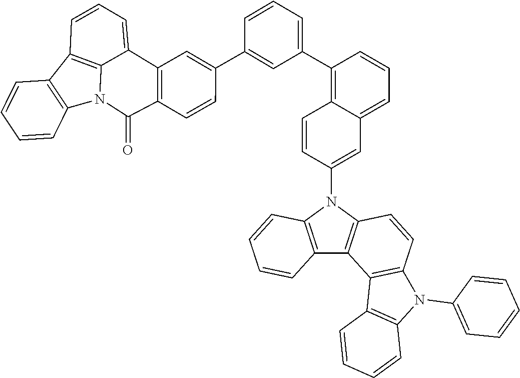 [SMILES: O=c1c2ccc(-c3cccc(-c4cccc5cc(-n6c7ccccc7c7c8c9ccccc9n(-c9ccccc9)c8ccc76)ccc45)c3)cc2c2cccc3c4ccccc4n1c23]

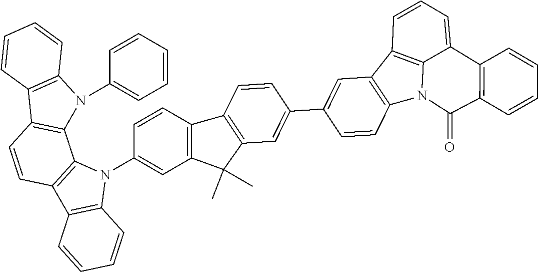 [SMILES: CC1(C)c2cc(-c3ccc4c(c3)c3cccc5c6ccccc6c(=O)n4c53)ccc2-c2ccc(-n3c4ccccc4c4ccc5c6ccccc6n(-c6ccccc6)c5c43)cc21]

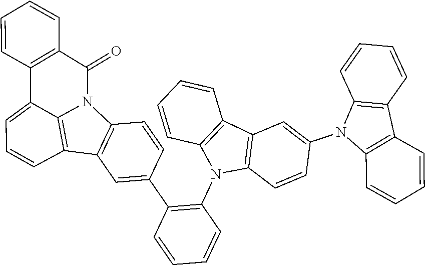 [SMILES: O=c1c2ccccc2c2cccc3c4cc(-c5ccccc5-n5c6ccccc6c6cc(-n7c8ccccc8c8ccccc87)ccc65)ccc4n1c23]

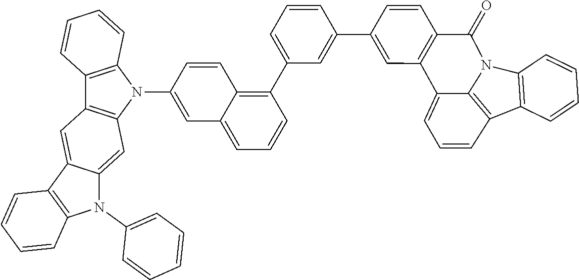 [SMILES: O=c1c2ccc(-c3cccc(-c4cccc5cc(-n6c7ccccc7c7cc8c9ccccc9n(-c9ccccc9)c8cc76)ccc45)c3)cc2c2cccc3c4ccccc4n1c23]

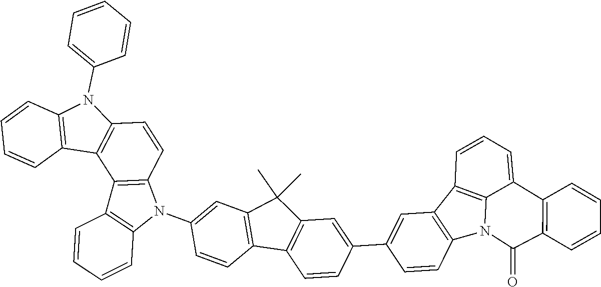 [SMILES: CC1(C)c2cc(-c3ccc4c(c3)c3cccc5c6ccccc6c(=O)n4c53)ccc2-c2ccc(-n3c4ccccc4c4c5c6ccccc6n(-c6ccccc6)c5ccc43)cc21]

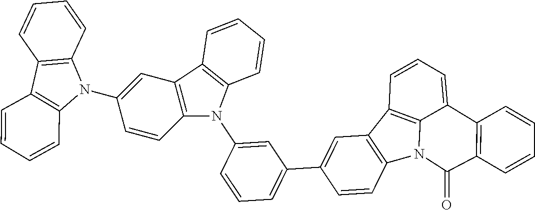 [SMILES: O=c1c2ccccc2c2cccc3c4cc(-c5cccc(-n6c7ccccc7c7cc(-n8c9ccccc9c9ccccc98)ccc76)c5)ccc4n1c23]

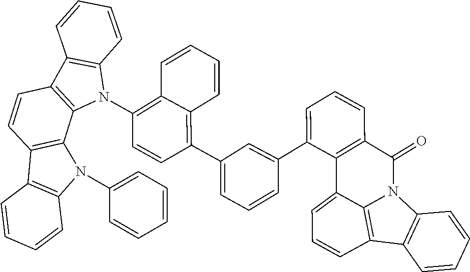 [SMILES: O=c1c2cccc(-c3cccc(-c4ccc(-n5c6ccccc6c6ccc7c8ccccc8n(-c8ccccc8)c7c65)c5ccccc45)c3)c2c2cccc3c4ccccc4n1c32]

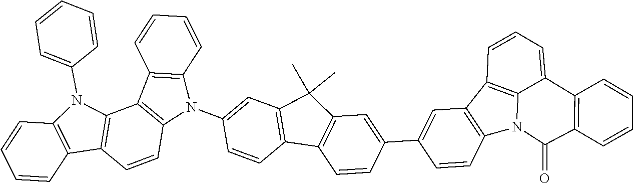 [SMILES: CC1(C)c2cc(-c3ccc4c(c3)c3cccc5c6ccccc6c(=O)n4c53)ccc2-c2ccc(-n3c4ccccc4c4c3ccc3c5ccccc5n(-c5ccccc5)c34)cc21]

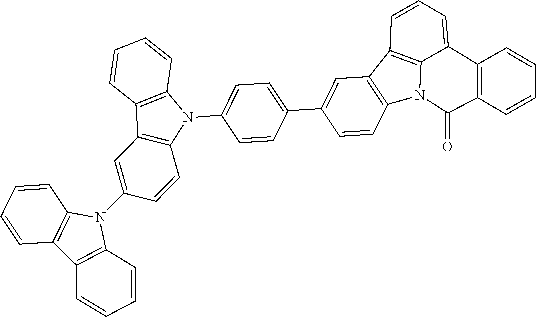 [SMILES: O=c1c2ccccc2c2cccc3c4cc(-c5ccc(-n6c7ccccc7c7cc(-n8c9ccccc9c9ccccc98)ccc76)cc5)ccc4n1c23]

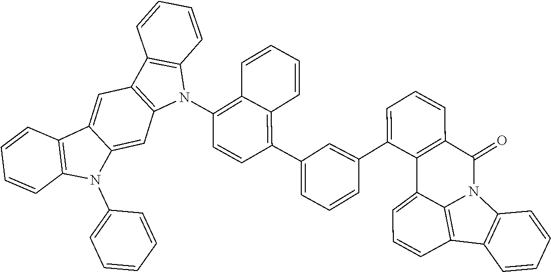 [SMILES: O=c1c2cccc(-c3cccc(-c4ccc(-n5c6ccccc6c6cc7c8ccccc8n(-c8ccccc8)c7cc65)c5ccccc45)c3)c2c2cccc3c4ccccc4n1c32]

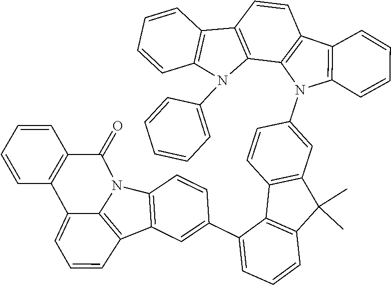 [SMILES: CC1(C)c2cc(-n3c4ccccc4c4ccc5c6ccccc6n(-c6ccccc6)c5c43)ccc2-c2c(-c3ccc4c(c3)c3cccc5c6ccccc6c(=O)n4c53)cccc21]

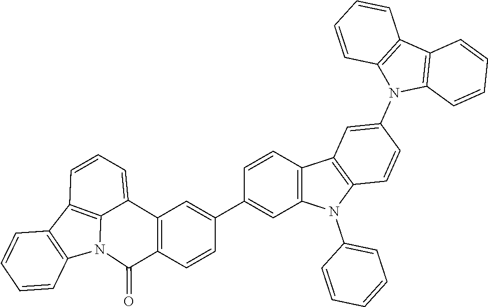 [SMILES: O=c1c2ccc(-c3ccc4c5cc(-n6c7ccccc7c7ccccc76)ccc5n(-c5ccccc5)c4c3)cc2c2cccc3c4ccccc4n1c23]

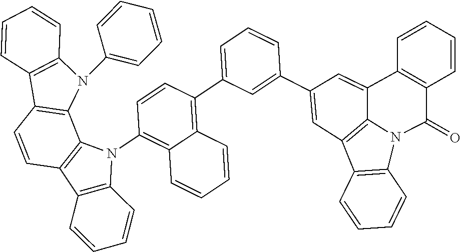 [SMILES: O=c1c2ccccc2c2cc(-c3cccc(-c4ccc(-n5c6ccccc6c6ccc7c8ccccc8n(-c8ccccc8)c7c65)c5ccccc45)c3)cc3c4ccccc4n1c23]

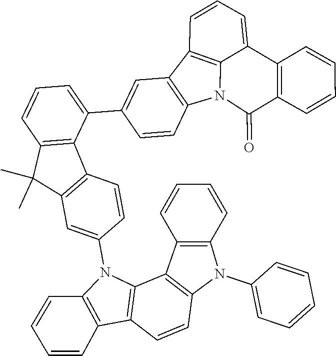 [SMILES: CC1(C)c2cc(-n3c4ccccc4c4ccc5c(c6ccccc6n5-c5ccccc5)c43)ccc2-c2c(-c3ccc4c(c3)c3cccc5c6ccccc6c(=O)n4c53)cccc21]

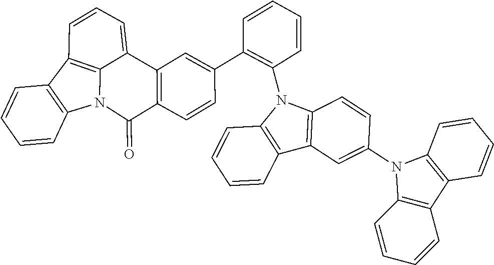 [SMILES: O=c1c2ccc(-c3ccccc3-n3c4ccccc4c4cc(-n5c6ccccc6c6ccccc65)ccc43)cc2c2cccc3c4ccccc4n1c23]

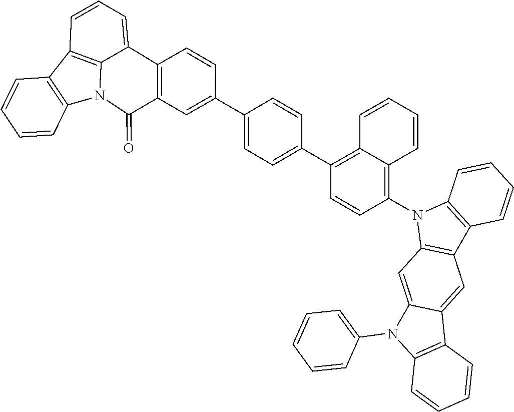 [SMILES: O=c1c2cc(-c3ccc(-c4ccc(-n5c6ccccc6c6cc7c8ccccc8n(-c8ccccc8)c7cc65)c5ccccc45)cc3)ccc2c2cccc3c4ccccc4n1c23]

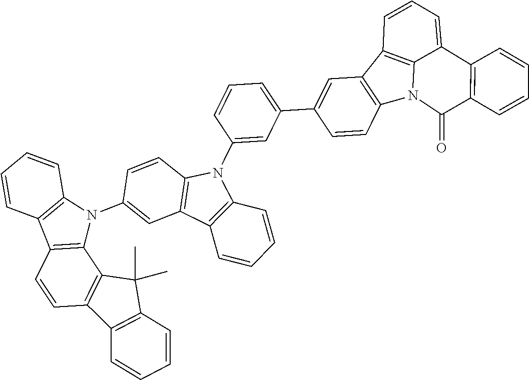 [SMILES: CC1(C)c2ccccc2-c2ccc3c4ccccc4n(-c4ccc5c(c4)c4ccccc4n5-c4cccc(-c5ccc6c(c5)c5cccc7c8ccccc8c(=O)n6c75)c4)c3c21]

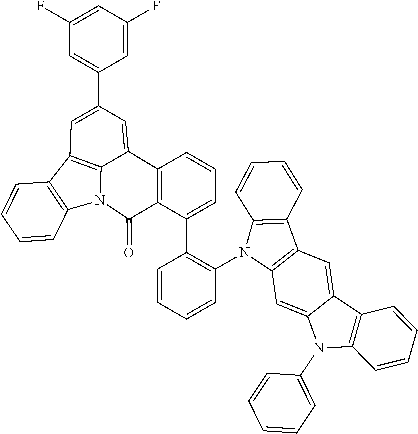 [SMILES: O=c1c2c(-c3ccccc3-n3c4ccccc4c4cc5c6ccccc6n(-c6ccccc6)c5cc43)cccc2c2cc(-c3cc(F)cc(F)c3)cc3c4ccccc4n1c23]

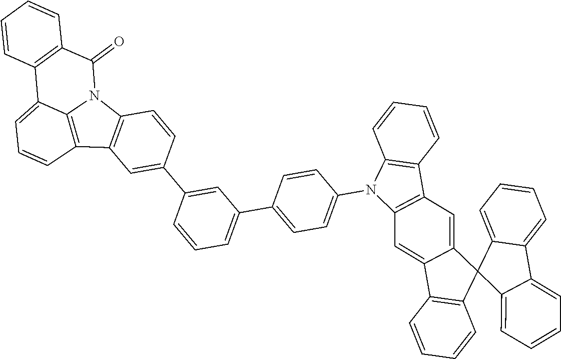 [SMILES: O=c1c2ccccc2c2cccc3c4cc(-c5cccc(-c6ccc(-n7c8ccccc8c8cc9c(cc87)-c7ccccc7C97c8ccccc8-c8ccccc87)cc6)c5)ccc4n1c23]

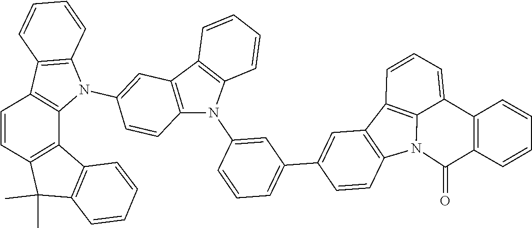 [SMILES: CC1(C)c2ccccc2-c2c1ccc1c3ccccc3n(-c3ccc4c(c3)c3ccccc3n4-c3cccc(-c4ccc5c(c4)c4cccc6c7ccccc7c(=O)n5c64)c3)c21]

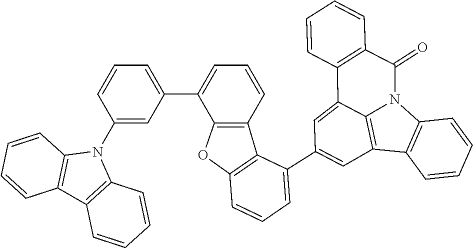 [SMILES: O=c1c2ccccc2c2cc(-c3cccc4oc5c(-c6cccc(-n7c8ccccc8c8ccccc87)c6)cccc5c34)cc3c4ccccc4n1c23]

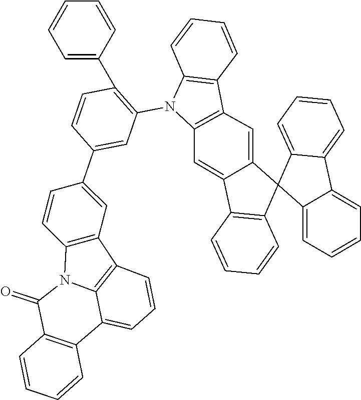 [SMILES: O=c1c2ccccc2c2cccc3c4cc(-c5ccc(-c6ccccc6)c(-n6c7ccccc7c7cc8c(cc76)-c6ccccc6C86c7ccccc7-c7ccccc76)c5)ccc4n1c23]

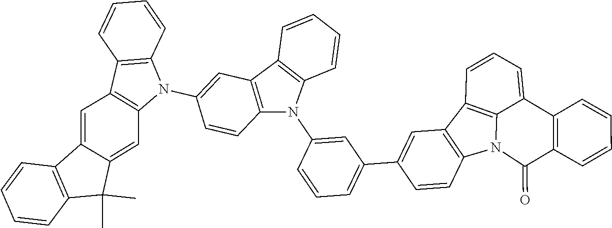 [SMILES: CC1(C)c2ccccc2-c2cc3c4ccccc4n(-c4ccc5c(c4)c4ccccc4n5-c4cccc(-c5ccc6c(c5)c5cccc7c8ccccc8c(=O)n6c75)c4)c3cc21]

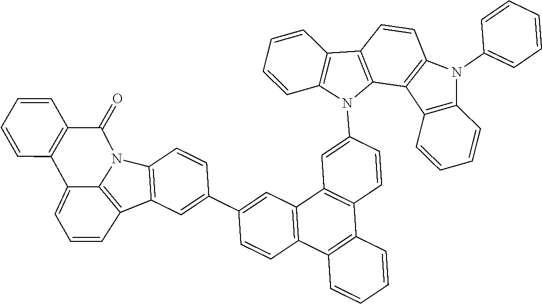 [SMILES: O=c1c2ccccc2c2cccc3c4cc(-c5ccc6c7ccccc7c7ccc(-n8c9ccccc9c9ccc%10c(c%11ccccc%11n%10-c%10ccccc%10)c98)cc7c6c5)ccc4n1c23]

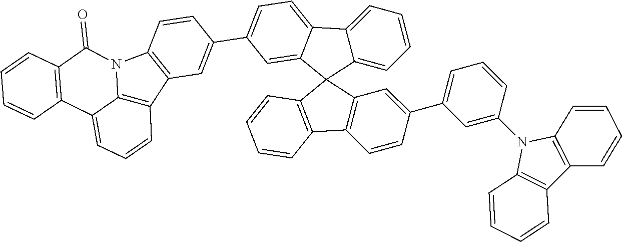 [SMILES: O=c1c2ccccc2c2cccc3c4cc(-c5ccc6c(c5)C5(c7ccccc7-c7ccc(-c8cccc(-n9c%10ccccc%10c%10ccccc%109)c8)cc75)c5ccccc5-6)ccc4n1c23]

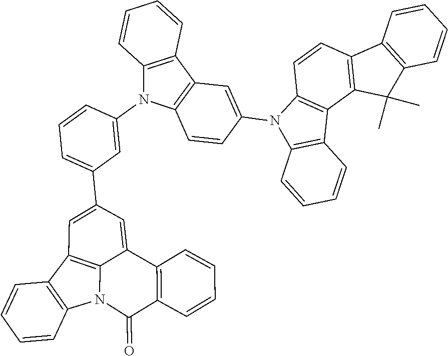 [SMILES: CC1(C)c2ccccc2-c2ccc3c(c21)c1ccccc1n3-c1ccc2c(c1)c1ccccc1n2-c1cccc(-c2cc3c4ccccc4c(=O)n4c5ccccc5c(c2)c34)c1]

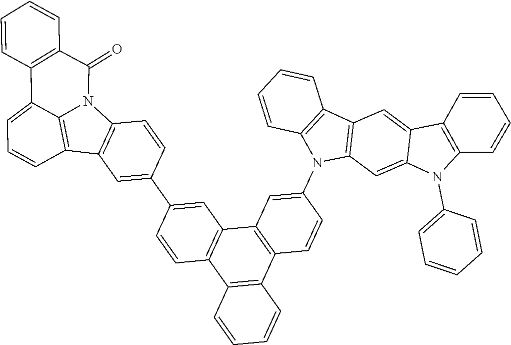 [SMILES: O=c1c2ccccc2c2cccc3c4cc(-c5ccc6c7ccccc7c7ccc(-n8c9ccccc9c9cc%10c%11ccccc%11n(-c%11ccccc%11)c%10cc98)cc7c6c5)ccc4n1c23]

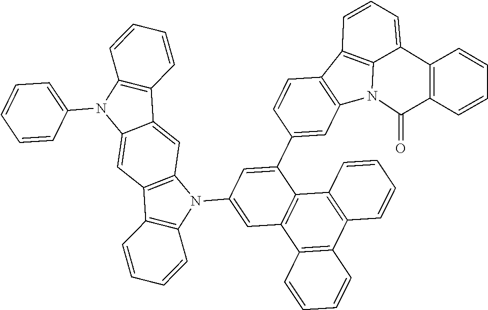 [SMILES: O=c1c2ccccc2c2cccc3c4ccc(-c5cc(-n6c7ccccc7c7cc8c(cc76)c6ccccc6n8-c6ccccc6)cc6c7ccccc7c7ccccc7c56)cc4n1c23]

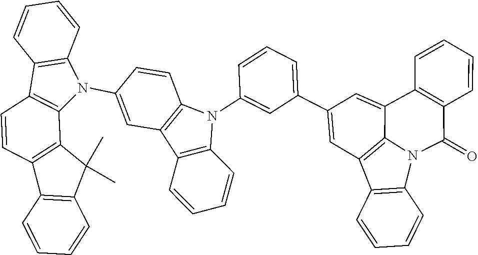 [SMILES: CC1(C)c2ccccc2-c2ccc3c4ccccc4n(-c4ccc5c(c4)c4ccccc4n5-c4cccc(-c5cc6c7ccccc7c(=O)n7c8ccccc8c(c5)c67)c4)c3c21]

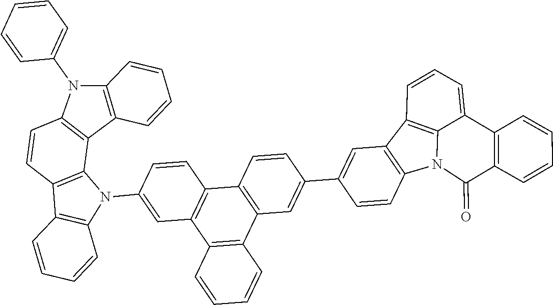 [SMILES: O=c1c2ccccc2c2cccc3c4cc(-c5ccc6c7ccc(-n8c9ccccc9c9ccc%10c(c%11ccccc%11n%10-c%10ccccc%10)c98)cc7c7ccccc7c6c5)ccc4n1c23]

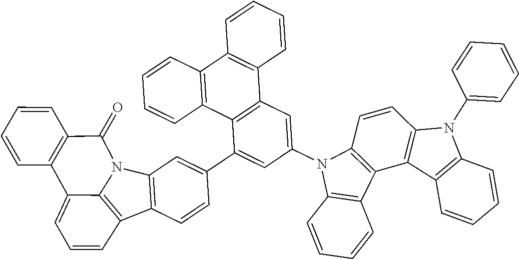 [SMILES: O=c1c2ccccc2c2cccc3c4ccc(-c5cc(-n6c7ccccc7c7c8c9ccccc9n(-c9ccccc9)c8ccc76)cc6c7ccccc7c7ccccc7c56)cc4n1c23]

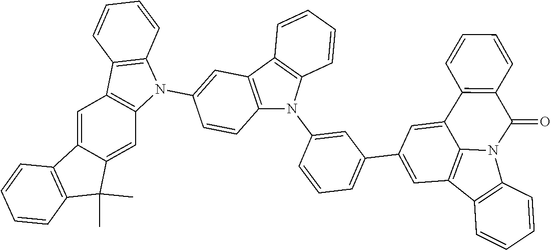 [SMILES: CC1(C)c2ccccc2-c2cc3c4ccccc4n(-c4ccc5c(c4)c4ccccc4n5-c4cccc(-c5cc6c7ccccc7c(=O)n7c8ccccc8c(c5)c67)c4)c3cc21]

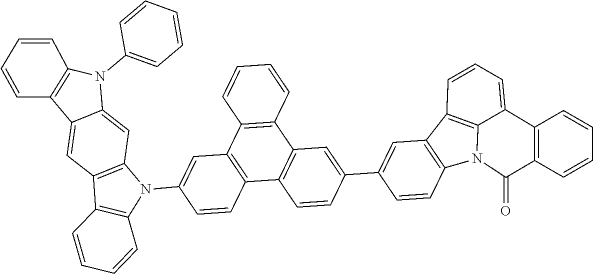 [SMILES: O=c1c2ccccc2c2cccc3c4cc(-c5ccc6c7ccc(-n8c9ccccc9c9cc%10c%11ccccc%11n(-c%11ccccc%11)c%10cc98)cc7c7ccccc7c6c5)ccc4n1c23]

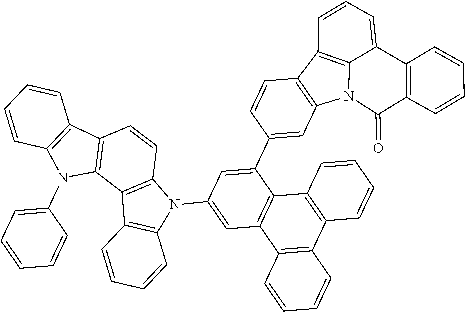 [SMILES: O=c1c2ccccc2c2cccc3c4ccc(-c5cc(-n6c7ccccc7c7c6ccc6c8ccccc8n(-c8ccccc8)c67)cc6c7ccccc7c7ccccc7c56)cc4n1c23]

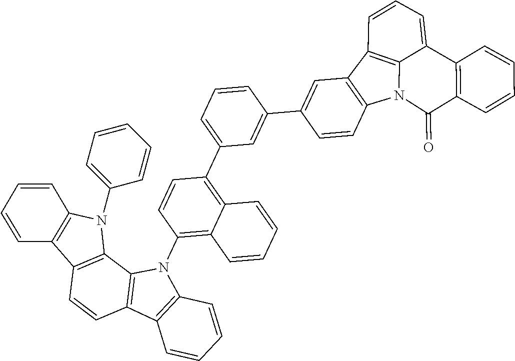 [SMILES: O=c1c2ccccc2c2cccc3c4cc(-c5cccc(-c6ccc(-n7c8ccccc8c8ccc9c%10ccccc%10n(-c%10ccccc%10)c9c87)c7ccccc67)c5)ccc4n1c23]

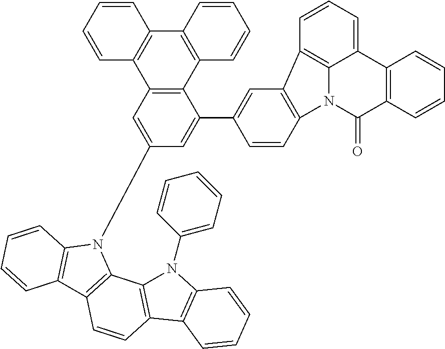 [SMILES: O=c1c2ccccc2c2cccc3c4cc(-c5cc(-n6c7ccccc7c7ccc8c9ccccc9n(-c9ccccc9)c8c76)cc6c7ccccc7c7ccccc7c56)ccc4n1c23]

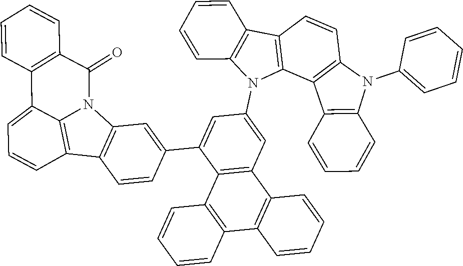 [SMILES: O=c1c2ccccc2c2cccc3c4ccc(-c5cc(-n6c7ccccc7c7ccc8c(c9ccccc9n8-c8ccccc8)c76)cc6c7ccccc7c7ccccc7c56)cc4n1c23]

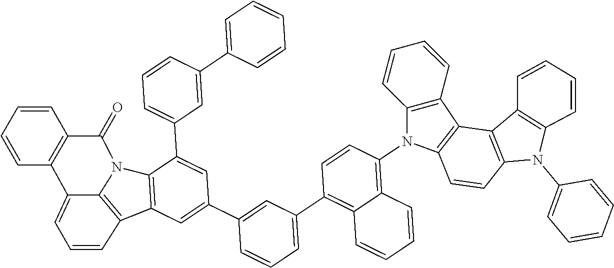 [SMILES: O=c1c2ccccc2c2cccc3c4cc(-c5cccc(-c6ccc(-n7c8ccccc8c8c9c%10ccccc%10n(-c%10ccccc%10)c9ccc87)c7ccccc67)c5)cc(-c5cccc(-c6ccccc6)c5)c4n1c23]

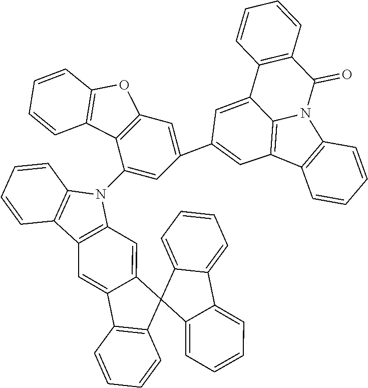 [SMILES: O=c1c2ccccc2c2cc(-c3cc(-n4c5ccccc5c5cc6c(cc54)C4(c5ccccc5-c5ccccc54)c4ccccc4-6)c4c(c3)oc3ccccc34)cc3c4ccccc4n1c23]

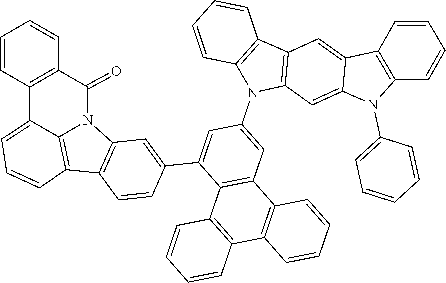 [SMILES: O=c1c2ccccc2c2cccc3c4ccc(-c5cc(-n6c7ccccc7c7cc8c9ccccc9n(-c9ccccc9)c8cc76)cc6c7ccccc7c7ccccc7c56)cc4n1c23]

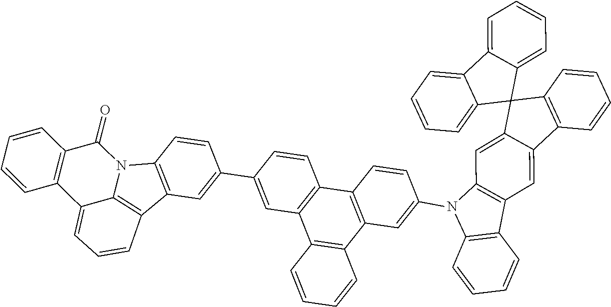 [SMILES: O=c1c2ccccc2c2cccc3c4cc(-c5ccc6c7ccc(-n8c9ccccc9c9cc%10c(cc98)C8(c9ccccc9-c9ccccc98)c8ccccc8-%10)cc7c7ccccc7c6c5)ccc4n1c23]

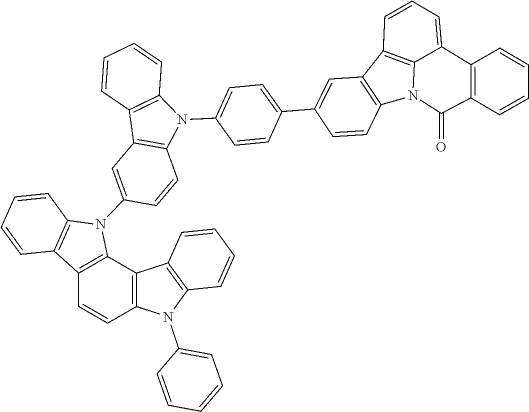 [SMILES: O=c1c2ccccc2c2cccc3c4cc(-c5ccc(-n6c7ccccc7c7cc(-n8c9ccccc9c9ccc%10c(c%11ccccc%11n%10-c%10ccccc%10)c98)ccc76)cc5)ccc4n1c23]

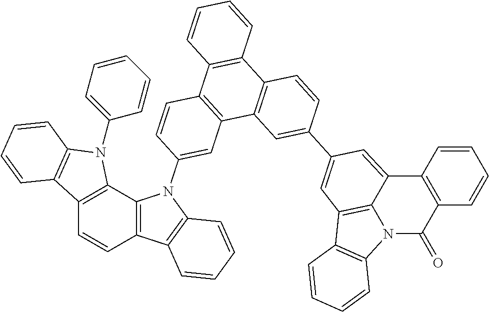 [SMILES: O=c1c2ccccc2c2cc(-c3ccc4c5ccccc5c5ccc(-n6c7ccccc7c7ccc8c9ccccc9n(-c9ccccc9)c8c76)cc5c4c3)cc3c4ccccc4n1c23]

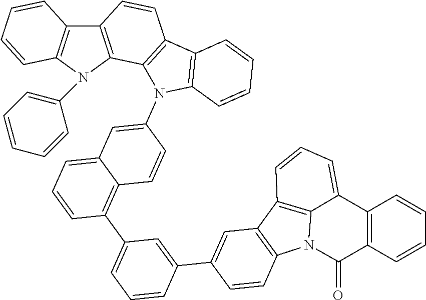 [SMILES: O=c1c2ccccc2c2cccc3c4cc(-c5cccc(-c6cccc7cc(-n8c9ccccc9c9ccc%10c%11ccccc%11n(-c%11ccccc%11)c%10c98)ccc67)c5)ccc4n1c23]

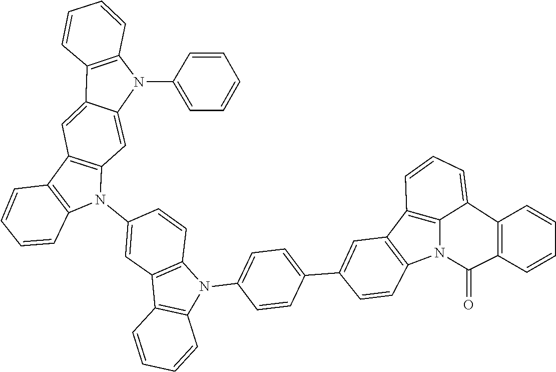 [SMILES: O=c1c2ccccc2c2cccc3c4cc(-c5ccc(-n6c7ccccc7c7cc(-n8c9ccccc9c9cc%10c%11ccccc%11n(-c%11ccccc%11)c%10cc98)ccc76)cc5)ccc4n1c23]

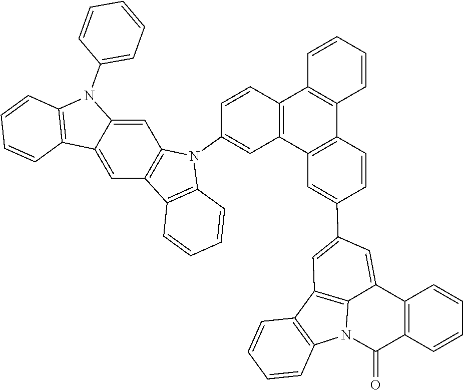 [SMILES: O=c1c2ccccc2c2cc(-c3ccc4c5ccccc5c5ccc(-n6c7ccccc7c7cc8c9ccccc9n(-c9ccccc9)c8cc76)cc5c4c3)cc3c4ccccc4n1c23]

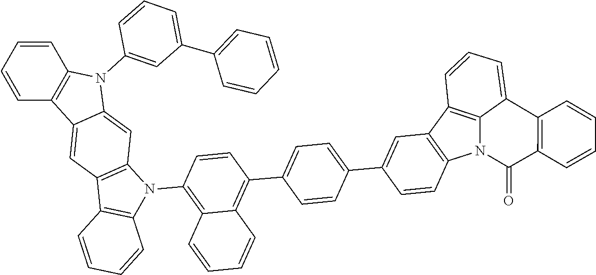 [SMILES: O=c1c2ccccc2c2cccc3c4cc(-c5ccc(-c6ccc(-n7c8ccccc8c8cc9c%10ccccc%10n(-c%10cccc(-c%11ccccc%11)c%10)c9cc87)c7ccccc67)cc5)ccc4n1c23]

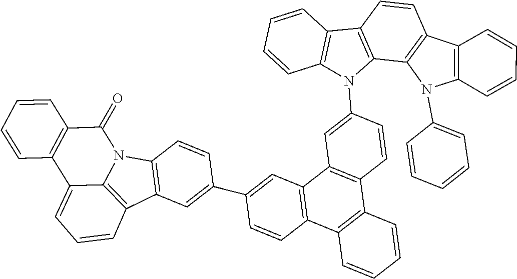 [SMILES: O=c1c2ccccc2c2cccc3c4cc(-c5ccc6c7ccccc7c7ccc(-n8c9ccccc9c9ccc%10c%11ccccc%11n(-c%11ccccc%11)c%10c98)cc7c6c5)ccc4n1c23]

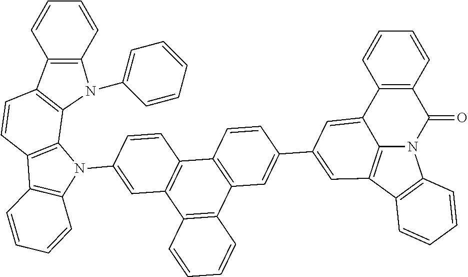 [SMILES: O=c1c2ccccc2c2cc(-c3ccc4c5ccc(-n6c7ccccc7c7ccc8c9ccccc9n(-c9ccccc9)c8c76)cc5c5ccccc5c4c3)cc3c4ccccc4n1c23]

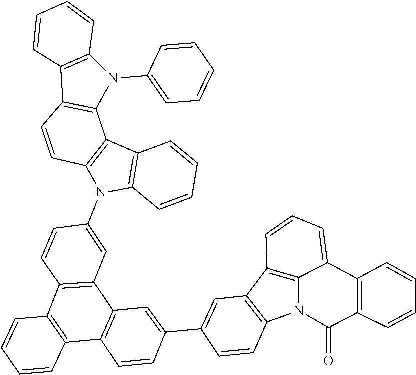 [SMILES: O=c1c2ccccc2c2cccc3c4cc(-c5ccc6c7ccccc7c7ccc(-n8c9ccccc9c9c8ccc8c%10ccccc%10n(-c%10ccccc%10)c89)cc7c6c5)ccc4n1c23]

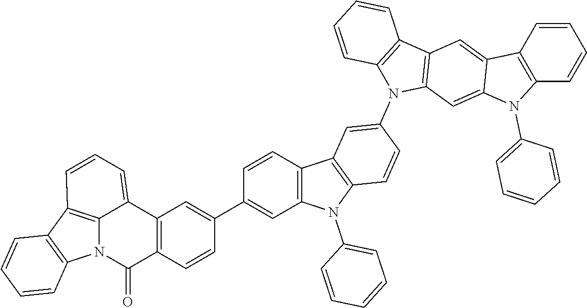 [SMILES: O=c1c2ccc(-c3ccc4c5cc(-n6c7ccccc7c7cc8c9ccccc9n(-c9ccccc9)c8cc76)ccc5n(-c5ccccc5)c4c3)cc2c2cccc3c4ccccc4n1c23]

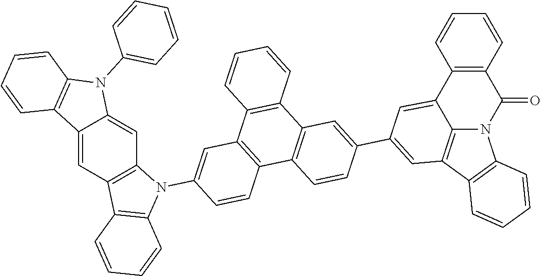 [SMILES: O=c1c2ccccc2c2cc(-c3ccc4c5ccc(-n6c7ccccc7c7cc8c9ccccc9n(-c9ccccc9)c8cc76)cc5c5ccccc5c4c3)cc3c4ccccc4n1c23]

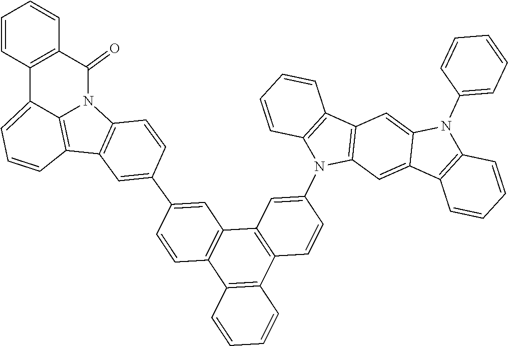 [SMILES: O=c1c2ccccc2c2cccc3c4cc(-c5ccc6c7ccccc7c7ccc(-n8c9ccccc9c9cc%10c(cc98)c8ccccc8n%10-c8ccccc8)cc7c6c5)ccc4n1c23]

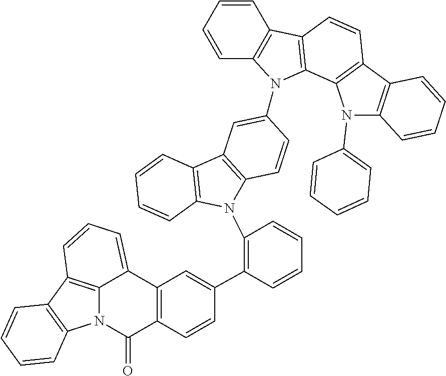 [SMILES: O=c1c2ccc(-c3ccccc3-n3c4ccccc4c4cc(-n5c6ccccc6c6ccc7c8ccccc8n(-c8ccccc8)c7c65)ccc43)cc2c2cccc3c4ccccc4n1c23]

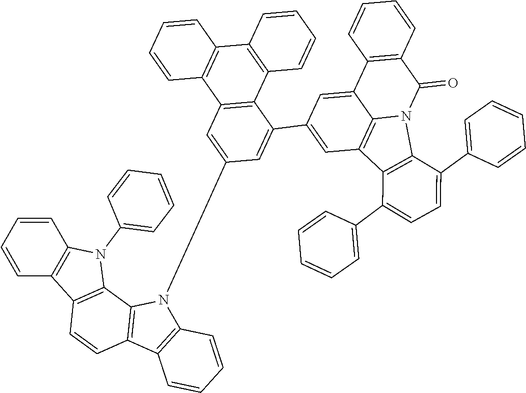 [SMILES: O=c1c2ccccc2c2cc(-c3cc(-n4c5ccccc5c5ccc6c7ccccc7n(-c7ccccc7)c6c54)cc4c5ccccc5c5ccccc5c34)cc3c4c(-c5ccccc5)ccc(-c5ccccc5)c4n1c23]

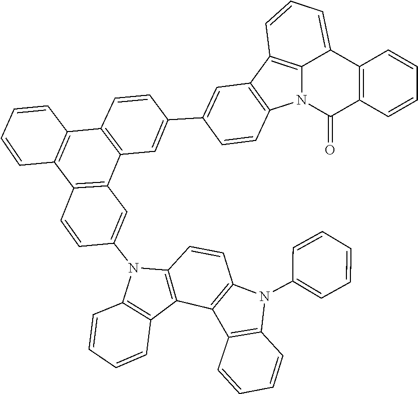 [SMILES: O=c1c2ccccc2c2cccc3c4cc(-c5ccc6c7ccccc7c7ccc(-n8c9ccccc9c9c%10c%11ccccc%11n(-c%11ccccc%11)c%10ccc98)cc7c6c5)ccc4n1c23]

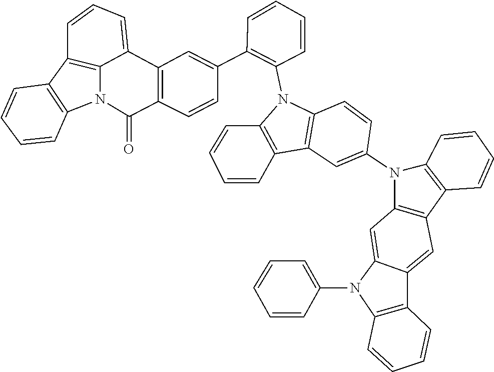 [SMILES: O=c1c2ccc(-c3ccccc3-n3c4ccccc4c4cc(-n5c6ccccc6c6cc7c8ccccc8n(-c8ccccc8)c7cc65)ccc43)cc2c2cccc3c4ccccc4n1c23]

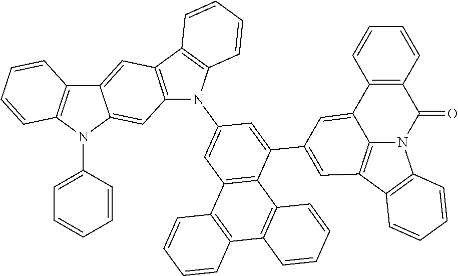 [SMILES: O=c1c2ccccc2c2cc(-c3cc(-n4c5ccccc5c5cc6c7ccccc7n(-c7ccccc7)c6cc54)cc4c5ccccc5c5ccccc5c34)cc3c4ccccc4n1c23]

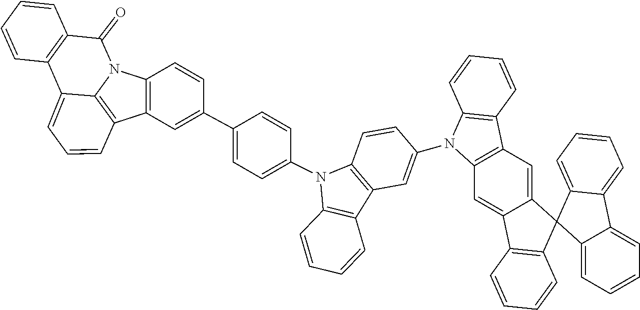 [SMILES: O=c1c2ccccc2c2cccc3c4cc(-c5ccc(-n6c7ccccc7c7cc(-n8c9ccccc9c9cc%10c(cc98)-c8ccccc8C%108c9ccccc9-c9ccccc98)ccc76)cc5)ccc4n1c23]

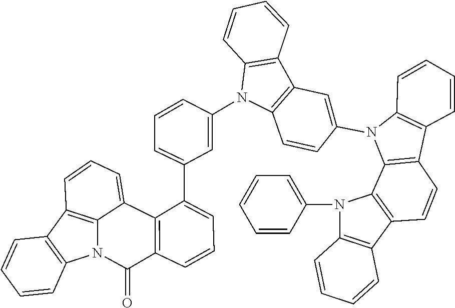 [SMILES: O=c1c2cccc(-c3cccc(-n4c5ccccc5c5cc(-n6c7ccccc7c7ccc8c9ccccc9n(-c9ccccc9)c8c76)ccc54)c3)c2c2cccc3c4ccccc4n1c32]

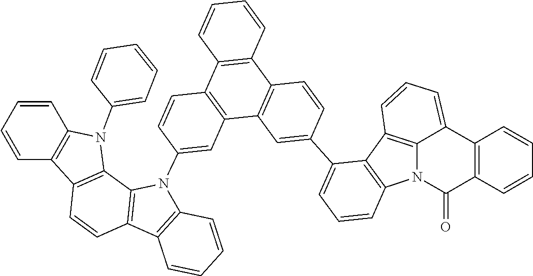 [SMILES: O=c1c2ccccc2c2cccc3c4c(-c5ccc6c7ccccc7c7ccc(-n8c9ccccc9c9ccc%10c%11ccccc%11n(-c%11ccccc%11)c%10c98)cc7c6c5)cccc4n1c23]

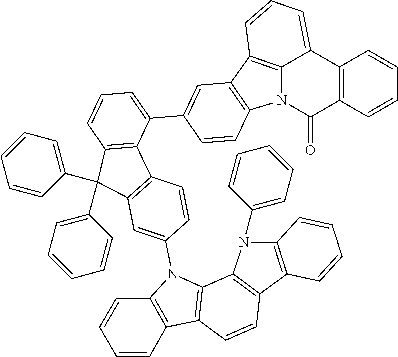 [SMILES: O=c1c2ccccc2c2cccc3c4cc(-c5cccc6c5-c5ccc(-n7c8ccccc8c8ccc9c%10ccccc%10n(-c%10ccccc%10)c9c87)cc5C6(c5ccccc5)c5ccccc5)ccc4n1c23]

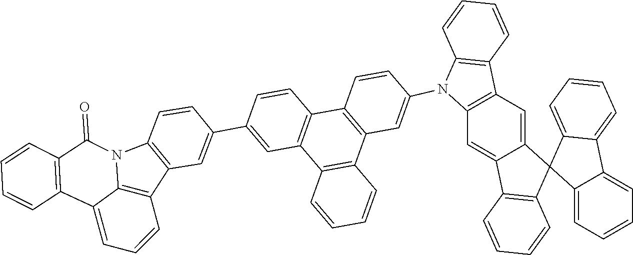 [SMILES: O=c1c2ccccc2c2cccc3c4cc(-c5ccc6c7ccc(-n8c9ccccc9c9cc%10c(cc98)-c8ccccc8C%108c9ccccc9-c9ccccc98)cc7c7ccccc7c6c5)ccc4n1c23]

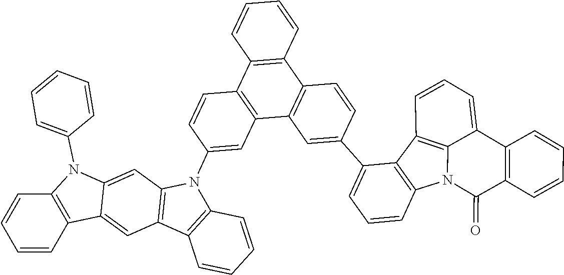 [SMILES: O=c1c2ccccc2c2cccc3c4c(-c5ccc6c7ccccc7c7ccc(-n8c9ccccc9c9cc%10c%11ccccc%11n(-c%11ccccc%11)c%10cc98)cc7c6c5)cccc4n1c23]

The base structure of the compounds of the invention can be prepared by the route outlined in schemes 1 to 3.

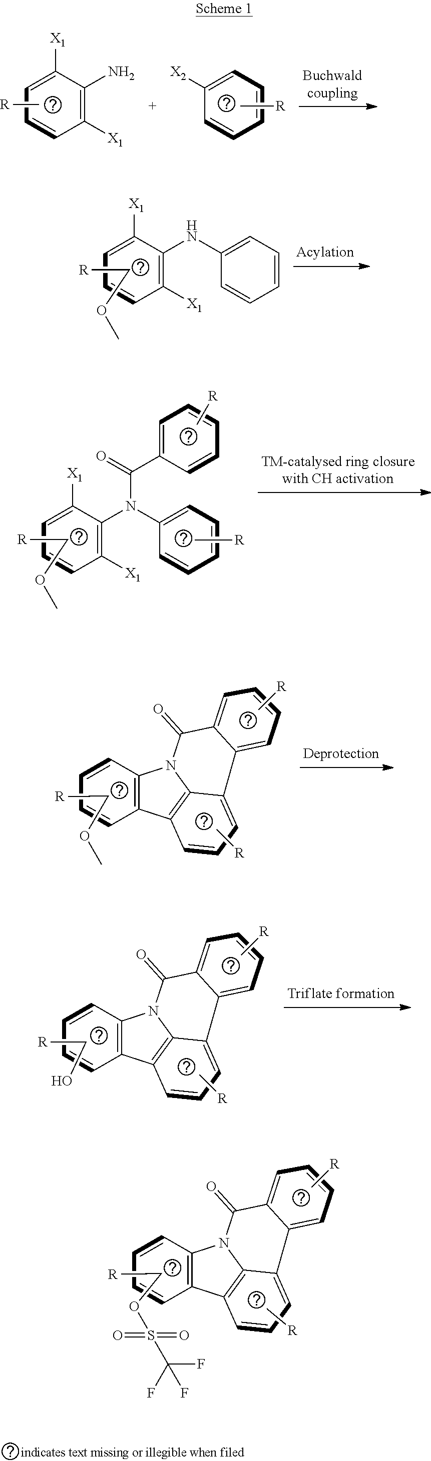

The two other base structures may also be prepared analogously (Schemes 2 and 3).

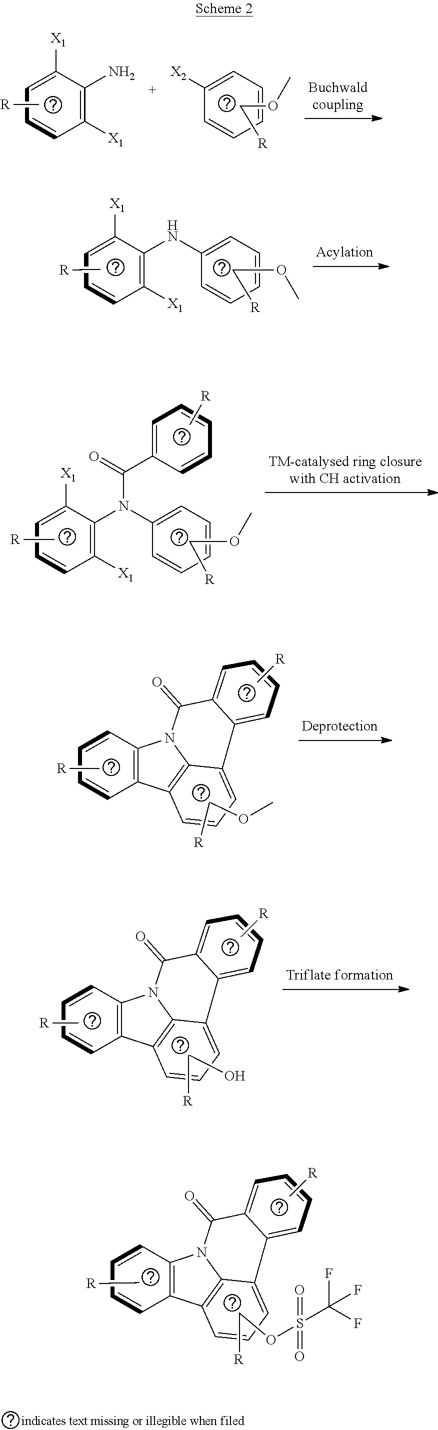

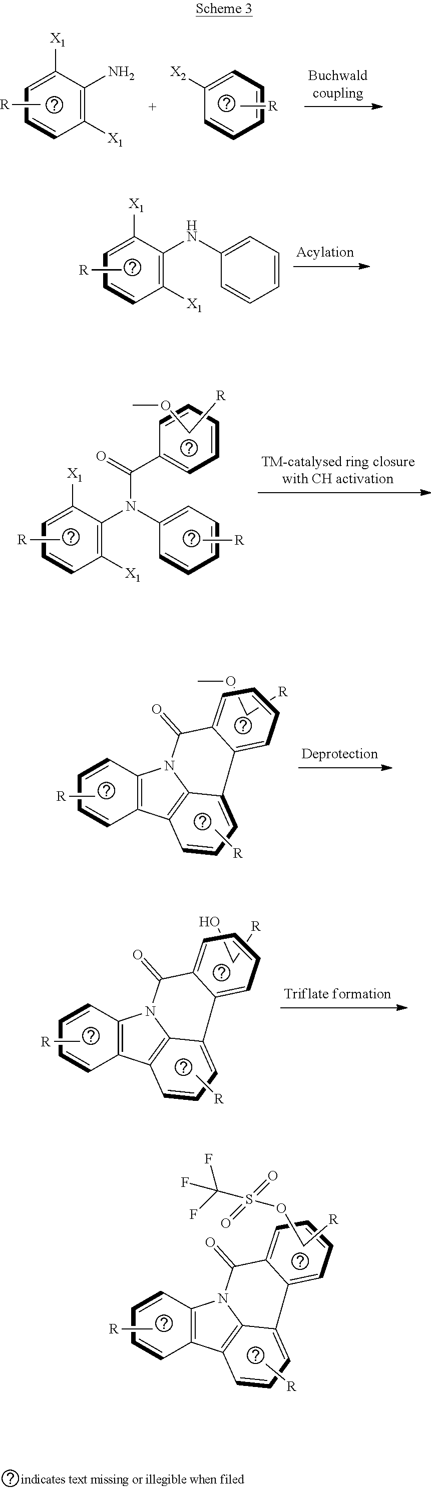

In Schemes 1 to 3, X₁═Cl or Br and X₂═Br or I, with the condition that, when X₁═Cl, X₂═Br and that, when X₁═Br, X₂═I.

The present invention therefore further provides a process for preparing the compounds of the invention, characterized by the following synthesis steps:
a) synthesizing the lactam base skeleton that bears a reactive leaving group, for example triflate or a halogen, rather than the Y group; and
b) introducing the Y group by a coupling reaction, for example a Suzuki coupling.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation comprising a compound of the invention and at least one further compound. The further compound may, for example, be a solvent, especially one of the abovementioned solvents or a mixture of these solvents. The further compound may alternatively be at least one further organic or inorganic compound which is likewise used in the electronic device, for example an emitting compound and/or a further matrix material. Suitable emitting compounds and further matrix materials are listed at the back in connection with the organic electroluminescent device. This further compound may also be polymeric.

The compounds of the invention are suitable for use in an electronic device, especially in an organic electroluminescent device.

The present invention therefore further provides for the use of a compound of the invention in an electronic device, especially in an organic electroluminescent device.

The present invention still further provides an electronic device comprising at least one compound of the invention.

An electronic device in the context of the present invention is a device comprising at least one layer comprising at least one organic compound.

This component may also comprise inorganic materials or else layers formed entirely from inorganic materials. The electronic device is preferably selected from the group consisting of organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), dye-sensitized organic solar cells (DSSCs), organic optical detectors, organic photoreceptors, organic field-quench devices (O-FQDs), light-emitting electrochemical cells (LECs), organic laser diodes (O-lasers) and organic plasmon emitting devices, preferably organic electroluminescent devices (OLEDs), more preferably phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem OLED, especially for white-emitting OLEDs.

The compound of the invention according to the above-detailed embodiments may be used in different layers, according to the exact structure. Preference is given to an organic electroluminescent device comprising a compound of formula (1) or the above-recited preferred embodiments in an emitting layer as matrix material for phosphorescent emitters or for emitters that exhibit TADF (thermally activated delayed fluorescence), especially for phosphorescent emitters. In this case, the organic electroluminescent device may contain an emitting layer, or it may contain a plurality of emitting layers, where at least one emitting layer contains at least one compound of the invention as matrix material. In addition, the compound of the invention can also be used in an electron transport layer and/or in a hole blocker layer and/or in a hole transport layer and/or in an exciton blocker layer.

When the compound of the invention is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the invention and the emitting compound contains between 99% and 1% by volume, preferably between 98% and 10% by volume, more preferably between 97% and 60% by volume and especially between 95% and 80% by volume of the compound of the invention, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

A further preferred embodiment of the present invention is the use of the compound of the invention as matrix material for a phosphorescent emitter in combination with a further matrix material. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, or dibenzofuran derivatives, for example according to WO 2015/169412, WO 2016/015810, WO 2016/023608, WO 2017/148564 or WO 2017/148565. It is likewise possible for a further phosphorescent emitter having shorter-wavelength emission than the actual emitter to be present as co-host in the mixture, or a compound not involved in charge transport to a significant extent, if at all, as described, for example, in WO 2010/108579. Such materials are preferably pure hydrocarbons. Examples of such materials can be found, for example, in WO 2006/130598, WO 2009/021126, WO 2009/124627 and WO 2010/006680.

Preferred matrix materials that can be used as a mixture together with the compounds of the invention are compounds of the following formulae (7) and (8):

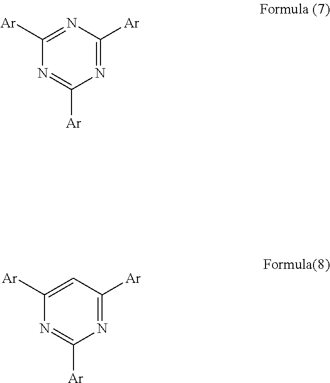

where Ar is the same or different at each instance and is an aromatic or heteroaromatic ring system which has 5 to 40 aromatic ring atoms and may be substituted by one or more R radicals, where R has the definitions given above.

Particular preference is given to the triazine derivatives of the formula (7).

In a preferred embodiment of the invention, the Ar group is the same or different at each instance and is selected from the group consisting of an aromatic or heteroaromatic ring system which has 6 to 30 aromatic ring atoms and may be substituted in each case by one or more R radicals. More preferably, the Ar groups are the same or different at each instance and are selected from the group consisting of an aromatic or heteroaromatic ring system which has 6 to 24 aromatic ring atoms, preferably 6 to 20 aromatic ring atoms, and may be substituted in each case by one or more R radicals, preferably nonaromatic R radicals.

Suitable aromatic or heteroaromatic ring systems Ar are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, quinazoline, benzimidazole, phenanthrene, triphenylene or a combination of two or three of these groups, each of which may be substituted by one or more R radicals.

The Ar groups here are preferably selected from the groups of the following formulae Ar-1 to Ar-83:

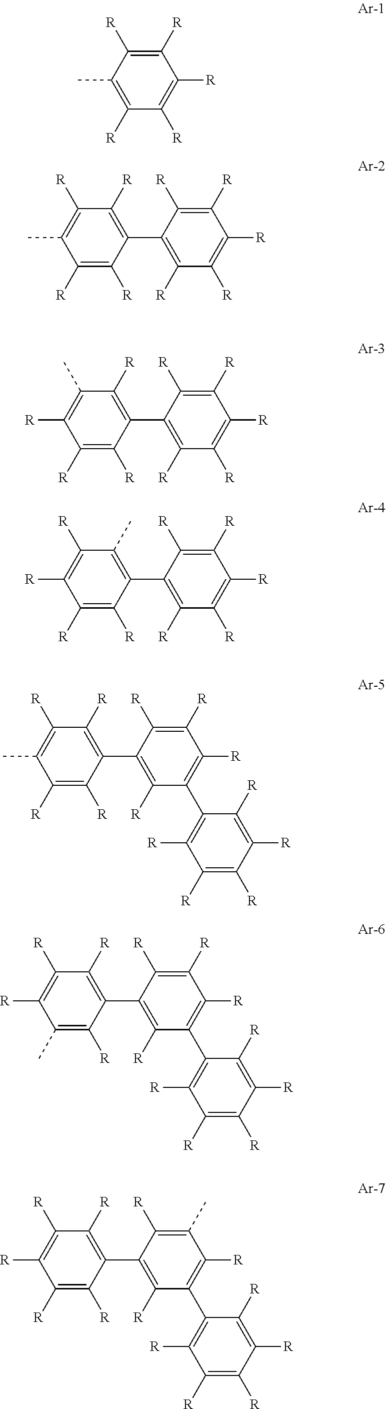

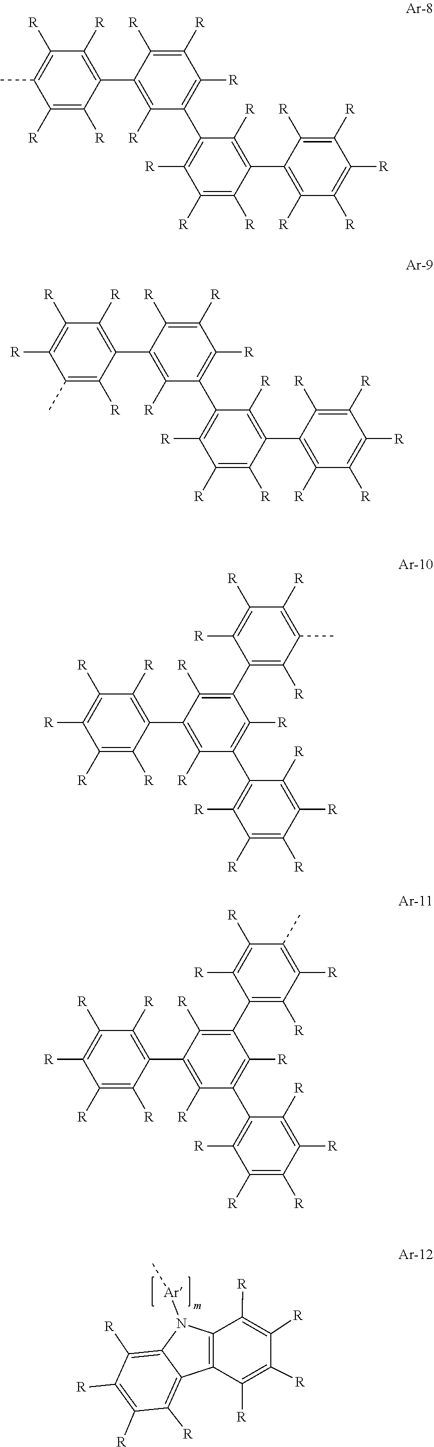

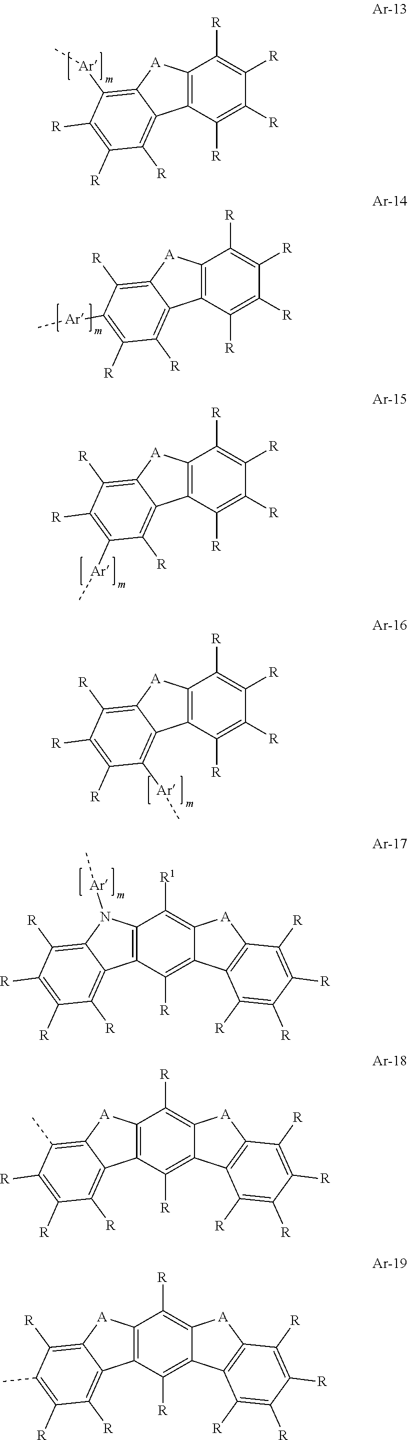

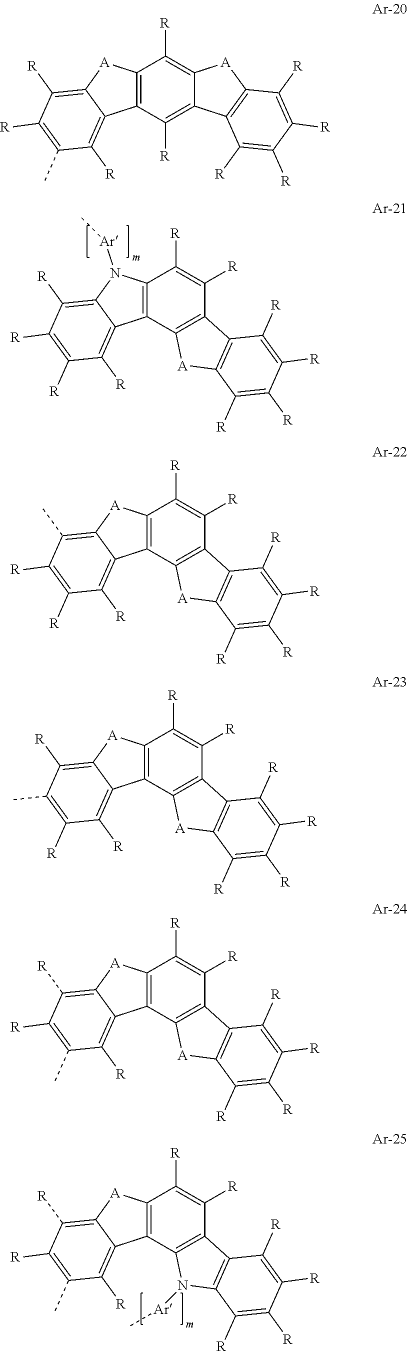

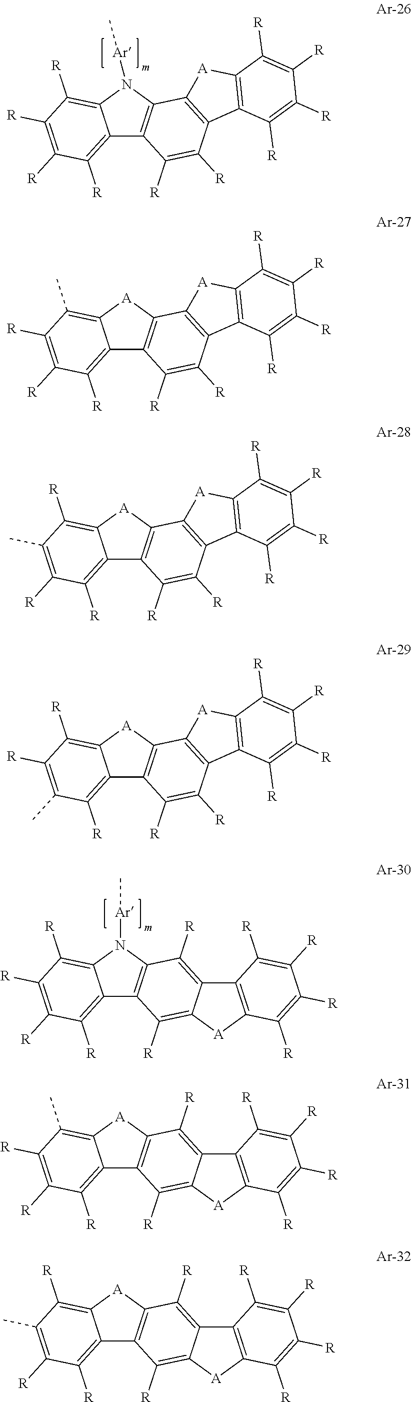

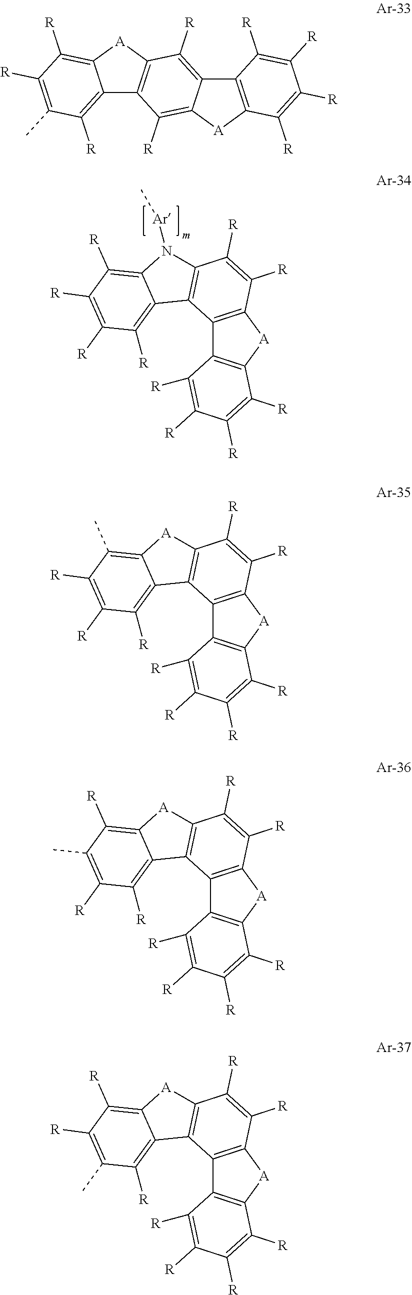

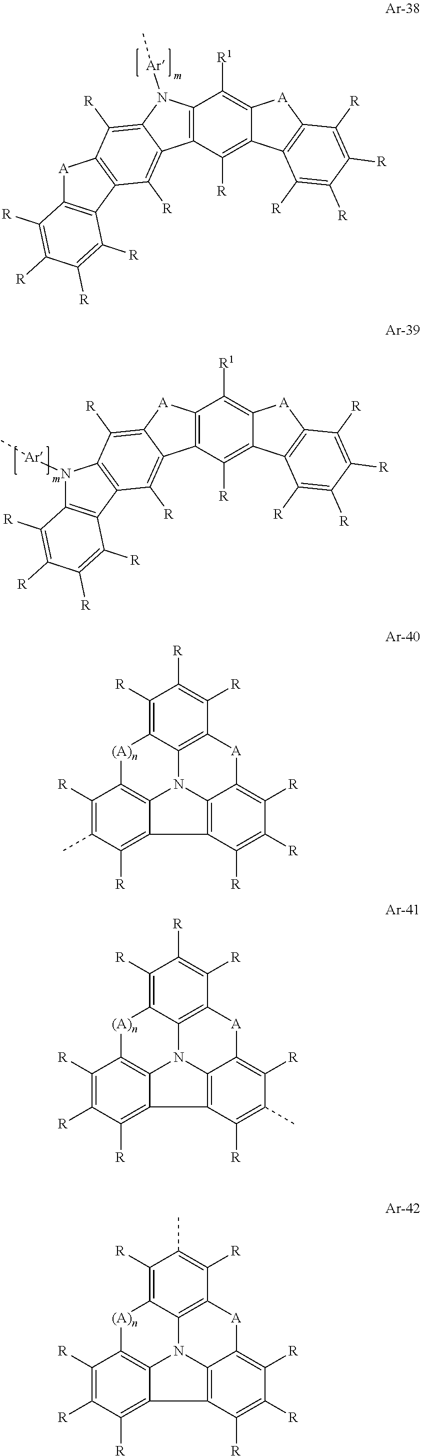

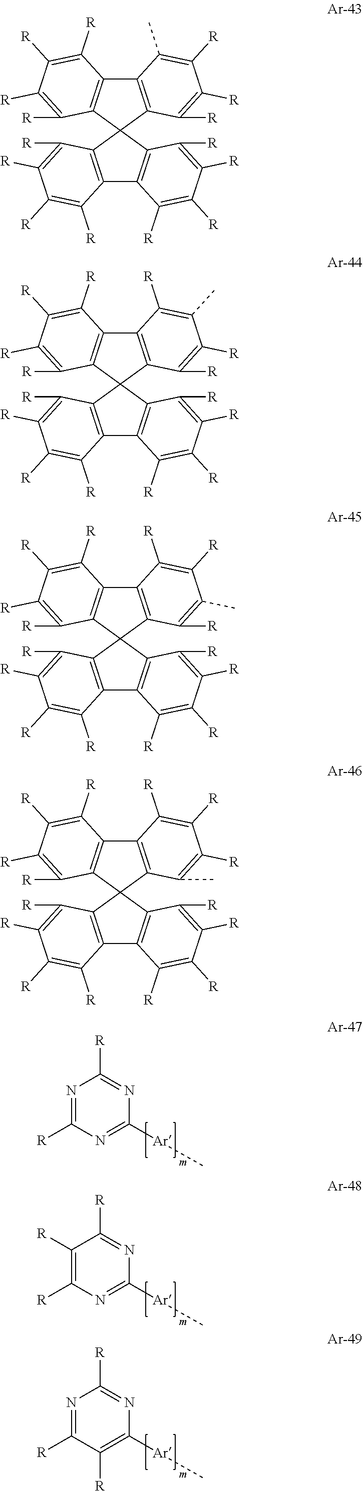

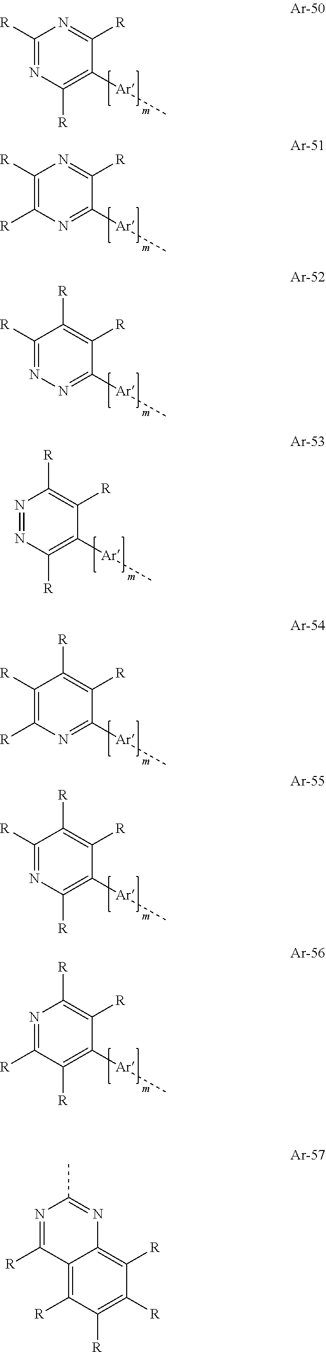

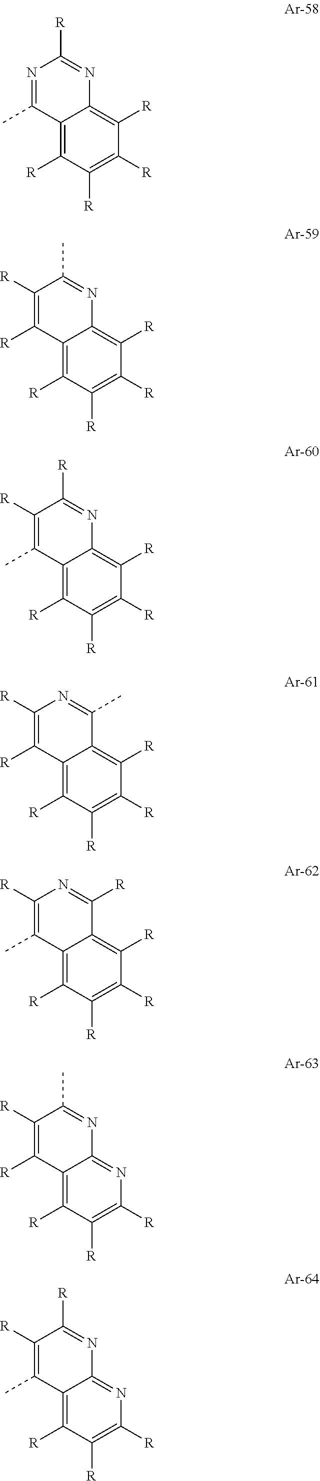

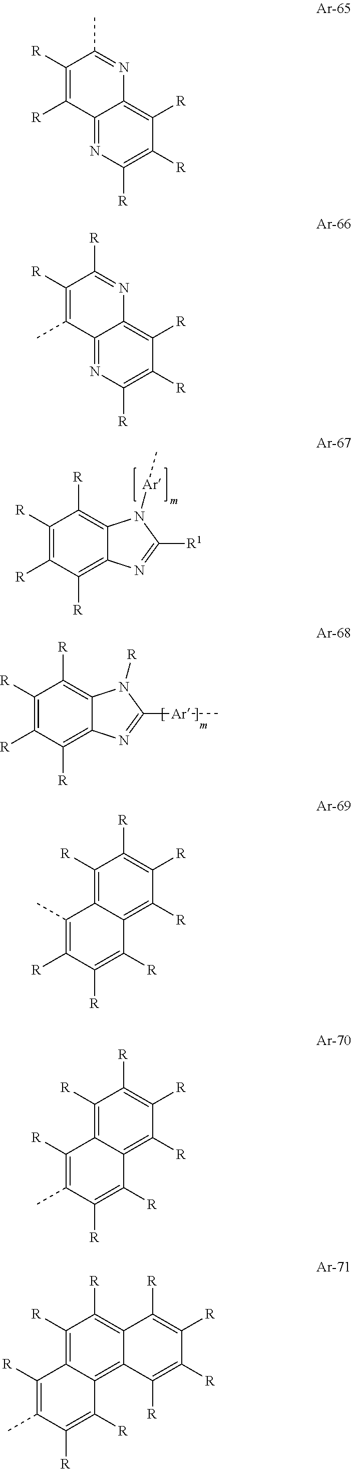

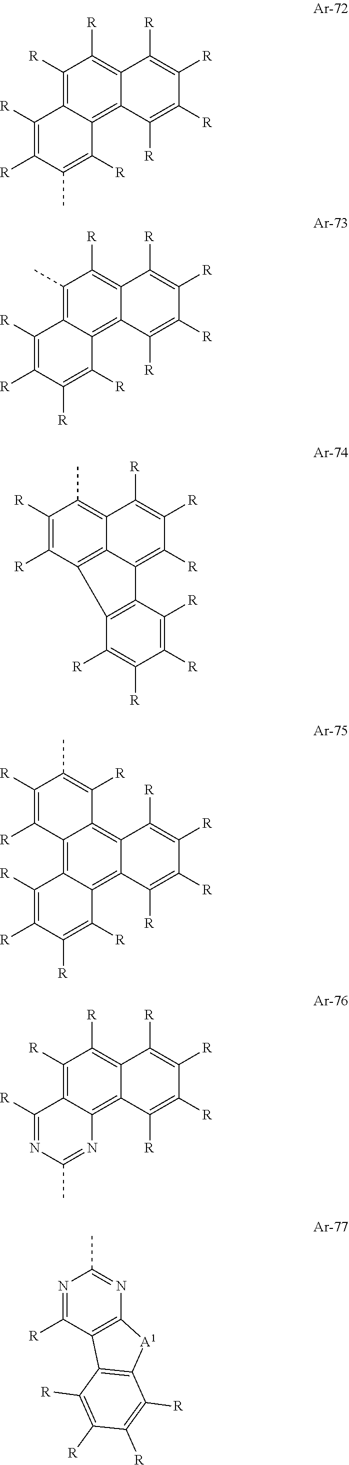

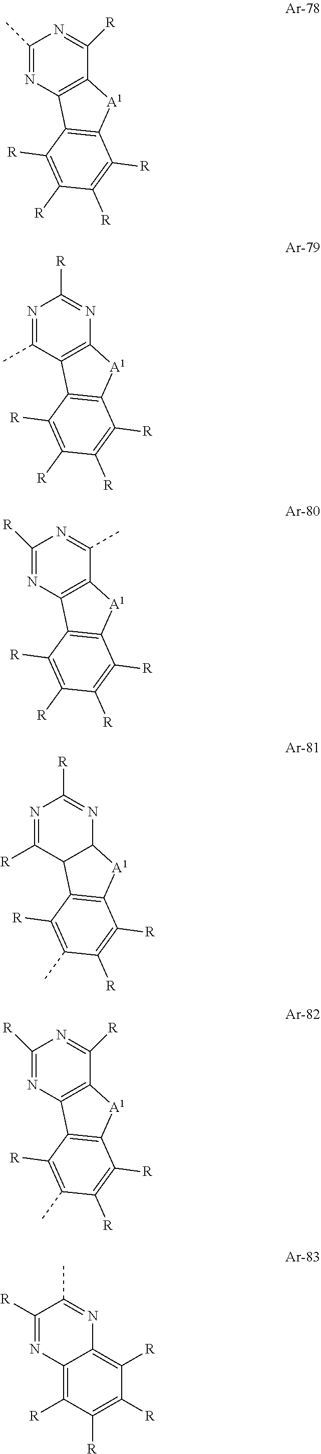

where R has the definitions given above, the dotted bond represents the bond to the triazine or pyrimidine and, in addition:
Ar′ is the same or different at each instance and is a bivalent aromatic or heteroaromatic ring system which has 6 to 18 aromatic ring atoms and may be substituted in each case by one or more R radicals;
A is the same or different at each instance and is CR₂, NR, O or S;
n is 0 or 1, where n=0 means that no A group is bonded at this position and R radicals are bonded to the corresponding carbon atoms instead;
m is 0 or 1, where m=0 means that the Ar′ group is absent and that the corresponding aromatic or heteroaromatic group is bonded directly to the triazine or pyrimidine.

The table that follows shows examples of suitable electron-transporting matrix materials that can be used together with the materials of the invention.

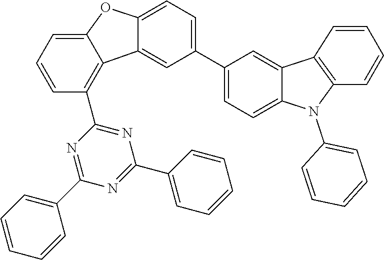 [SMILES: c1ccc(-c2nc(-c3ccccc3)nc(-c3cccc4oc5ccc(-c6ccc7c(c6)c6ccccc6n7-c6ccccc6)cc5c34)n2)cc1]

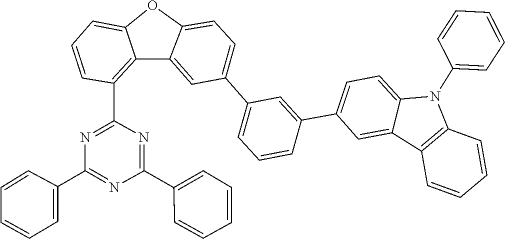 [SMILES: c1ccc(-c2nc(-c3ccccc3)nc(-c3cccc4oc5ccc(-c6cccc(-c7ccc8c(c7)c7ccccc7n8-c7ccccc7)c6)cc5c34)n2)cc1]

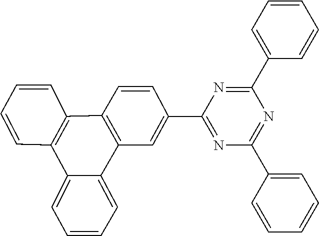 [SMILES: c1ccc(-c2nc(-c3ccccc3)nc(-c3ccc4c5ccccc5c5ccccc5c4c3)n2)cc1]

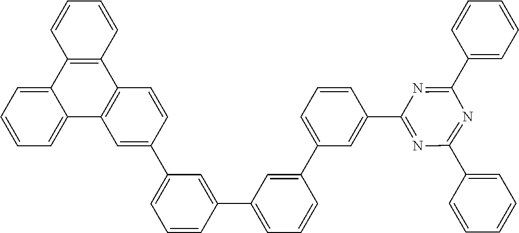 [SMILES: c1ccc(-c2nc(-c3ccccc3)nc(-c3cccc(-c4cccc(-c5cccc(-c6ccc7c8ccccc8c8ccccc8c7c6)c5)c4)c3)n2)cc1]

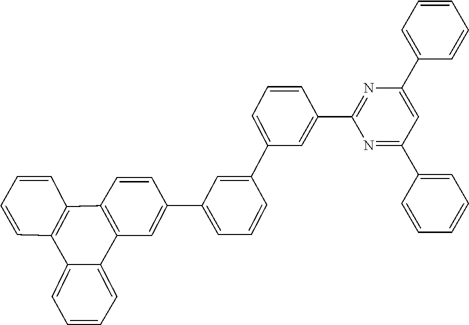 [SMILES: c1ccc(-c2cc(-c3ccccc3)nc(-c3cccc(-c4cccc(-c5ccc6c7ccccc7c7ccccc7c6c5)c4)c3)n2)cc1]

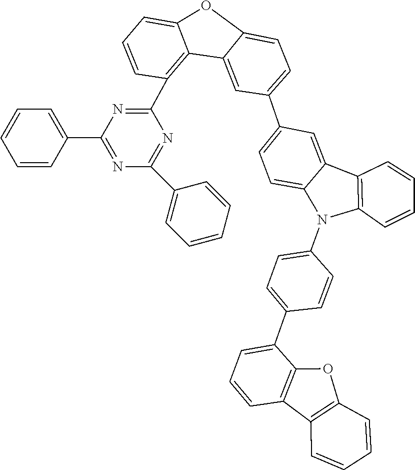 [SMILES: c1ccc(-c2nc(-c3ccccc3)nc(-c3cccc4oc5ccc(-c6ccc7c(c6)c6ccccc6n7-c6ccc(-c7cccc8c7oc7ccccc78)cc6)cc5c34)n2)cc1]

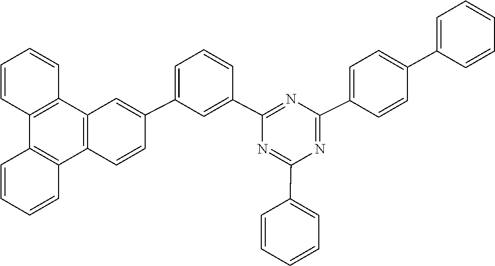 [SMILES: c1ccc(-c2ccc(-c3nc(-c4ccccc4)nc(-c4cccc(-c5ccc6c7ccccc7c7ccccc7c6c5)c4)n3)cc2)cc1]

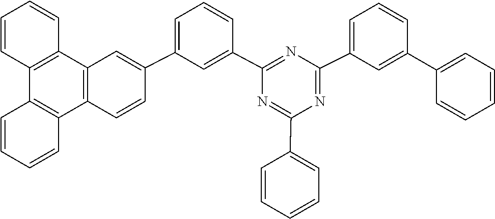 [SMILES: c1ccc(-c2cccc(-c3nc(-c4ccccc4)nc(-c4cccc(-c5ccc6c7ccccc7c7ccccc7c6c5)c4)n3)c2)cc1]

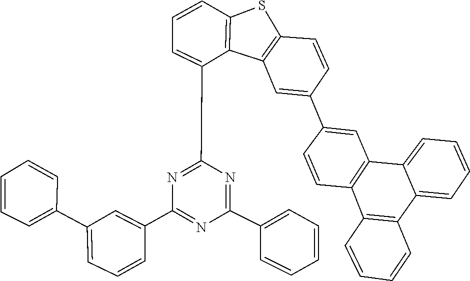 [SMILES: c1ccc(-c2cccc(-c3nc(-c4ccccc4)nc(-c4cccc5sc6ccc(-c7ccc8c9ccccc9c9ccccc9c8c7)cc6c45)n3)c2)cc1]

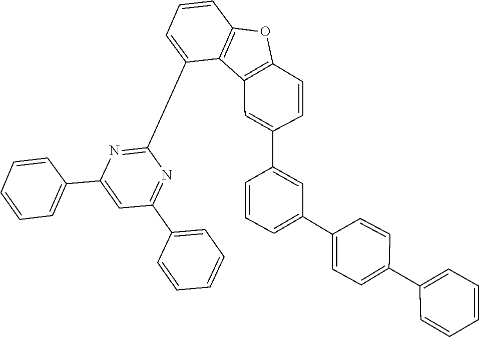 [SMILES: c1ccc(-c2ccc(-c3cccc(-c4ccc5oc6cccc(-c7nc(-c8ccccc8)cc(-c8ccccc8)n7)c6c5c4)c3)cc2)cc1]

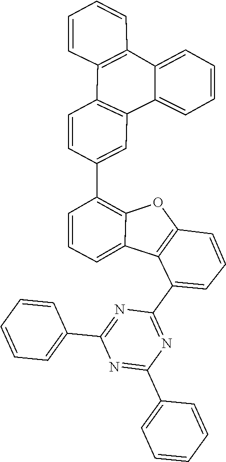 [SMILES: c1ccc(-c2nc(-c3ccccc3)nc(-c3cccc4oc5c(-c6ccc7c8ccccc8c8ccccc8c7c6)cccc5c34)n2)cc1]

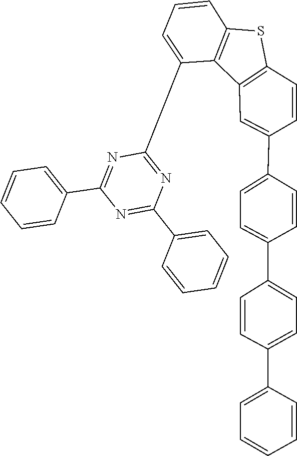 [SMILES: c1ccc(-c2ccc(-c3ccc(-c4ccc5sc6cccc(-c7nc(-c8ccccc8)nc(-c8ccccc8)n7)c6c5c4)cc3)cc2)cc1]

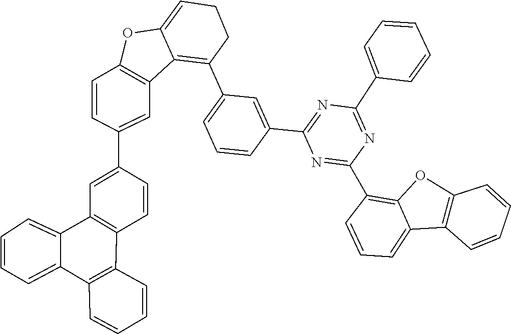 [SMILES: C1=c2oc3ccc(-c4ccc5c6ccccc6c6ccccc6c5c4)cc3c2=C(c2cccc(-c3nc(-c4ccccc4)nc(-c4cccc5c4oc4ccccc45)n3)c2)CC1]

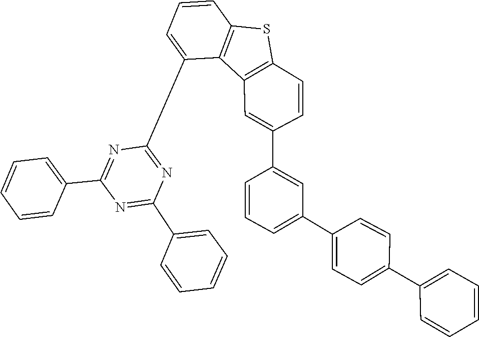 [SMILES: c1ccc(-c2ccc(-c3cccc(-c4ccc5sc6cccc(-c7nc(-c8ccccc8)nc(-c8ccccc8)n7)c6c5c4)c3)cc2)cc1]

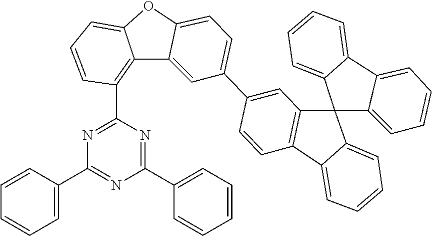 [SMILES: c1ccc(-c2nc(-c3ccccc3)nc(-c3cccc4oc5ccc(-c6ccc7c(c6)C6(c8ccccc8-c8ccccc86)c6ccccc6-7)cc5c34)n2)cc1]

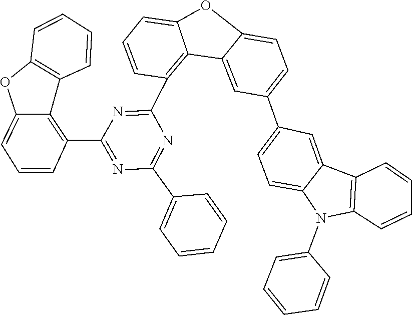 [SMILES: c1ccc(-c2nc(-c3cccc4oc5ccccc5c34)nc(-c3cccc4oc5ccc(-c6ccc7c(c6)c6ccccc6n7-c6ccccc6)cc5c34)n2)cc1]

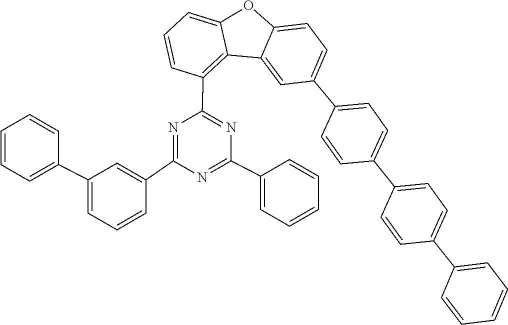 [SMILES: c1ccc(-c2ccc(-c3ccc(-c4ccc5oc6cccc(-c7nc(-c8ccccc8)nc(-c8cccc(-c9ccccc9)c8)n7)c6c5c4)cc3)cc2)cc1]

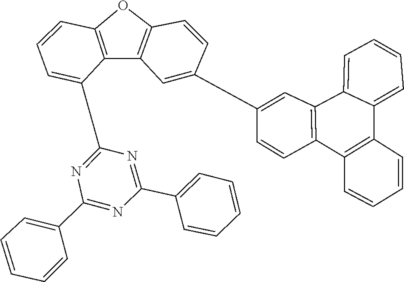 [SMILES: c1ccc(-c2nc(-c3ccccc3)nc(-c3cccc4oc5ccc(-c6ccc7c8ccccc8c8ccccc8c7c6)cc5c34)n2)cc1]

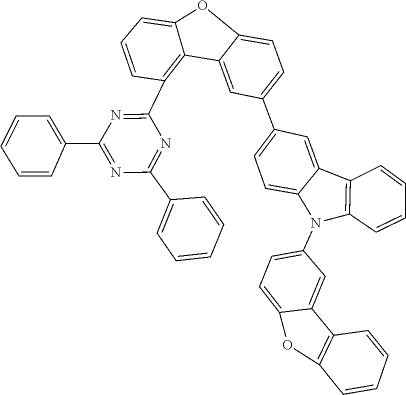 [SMILES: c1ccc(-c2nc(-c3ccccc3)nc(-c3cccc4oc5ccc(-c6ccc7c(c6)c6ccccc6n7-c6ccc7oc8ccccc8c7c6)cc5c34)n2)cc1]

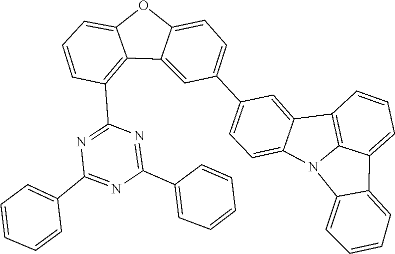 [SMILES: c1ccc(-c2nc(-c3ccccc3)nc(-c3cccc4oc5ccc(-c6ccc7c(c6)c6cccc8c9ccccc9n7c86)cc5c34)n2)cc1]

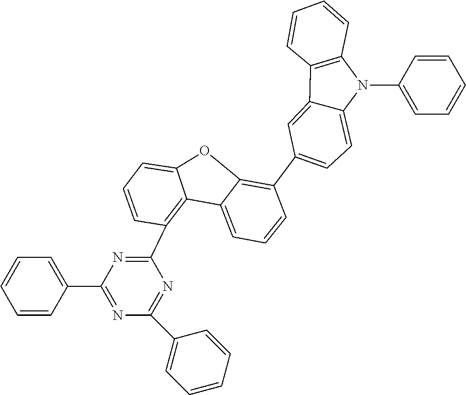 [SMILES: c1ccc(-c2nc(-c3ccccc3)nc(-c3cccc4oc5c(-c6ccc7c(c6)c6ccccc6n7-c6ccccc6)cccc5c34)n2)cc1]

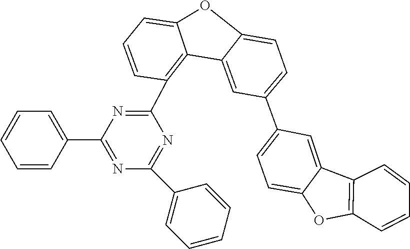

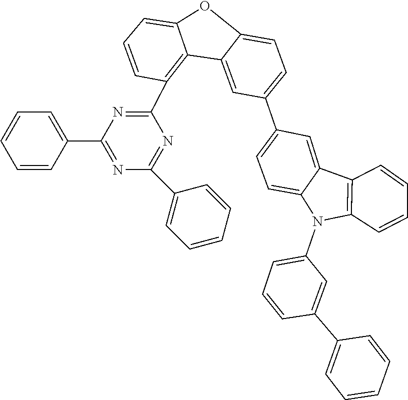

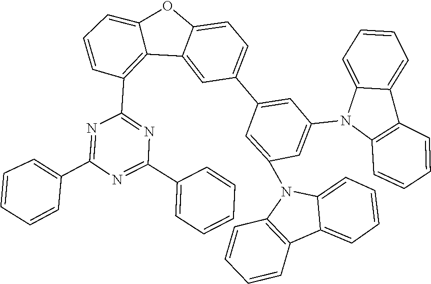 [SMILES: c1ccc(-c2nc(-c3ccccc3)nc(-c3cccc4oc5ccc(-c6cc(-n7c8ccccc8c8ccccc87)cc(-n7c8ccccc8c8ccccc87)c6)cc5c34)n2)cc1]

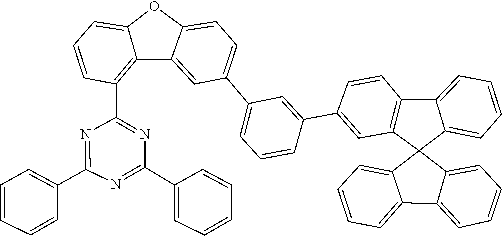 [SMILES: c1ccc(-c2nc(-c3ccccc3)nc(-c3cccc4oc5ccc(-c6cccc(-c7ccc8c(c7)C7(c9ccccc9-c9ccccc97)c7ccccc7-8)c6)cc5c34)n2)cc1]

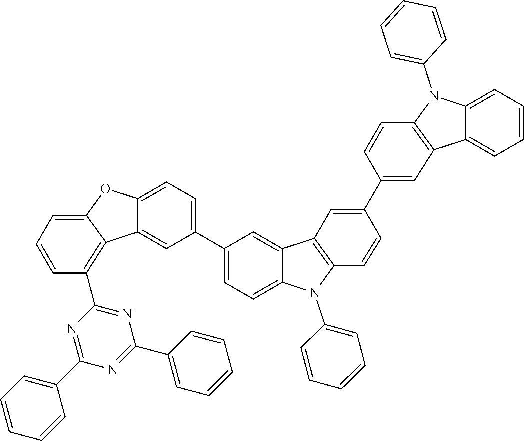 [SMILES: c1ccc(-c2nc(-c3ccccc3)nc(-c3cccc4oc5ccc(-c6ccc7c(c6)c6cc(-c8ccc9c(c8)c8ccccc8n9-c8ccccc8)ccc6n7-c6ccccc6)cc5c34)n2)cc1]

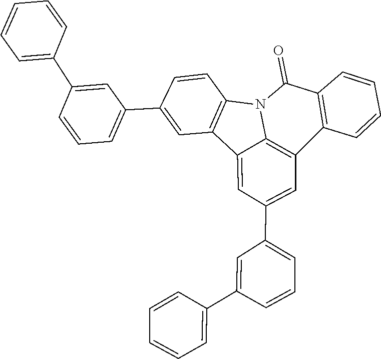 [SMILES: O=c1c2ccccc2c2cc(-c3cccc(-c4ccccc4)c3)cc3c4cc(-c5cccc(-c6ccccc6)c5)ccc4n1c23]

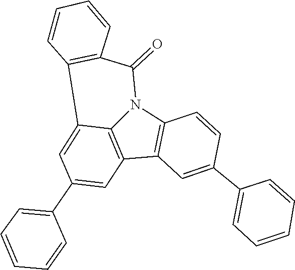 [SMILES: O=c1c2ccccc2c2cc(-c3ccccc3)cc3c4cc(-c5ccccc5)ccc4n1c23]

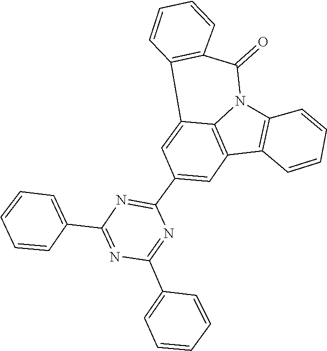 [SMILES: O=c1c2ccccc2c2cc(-c3nc(-c4ccccc4)nc(-c4ccccc4)n3)cc3c4ccccc4n1c23]

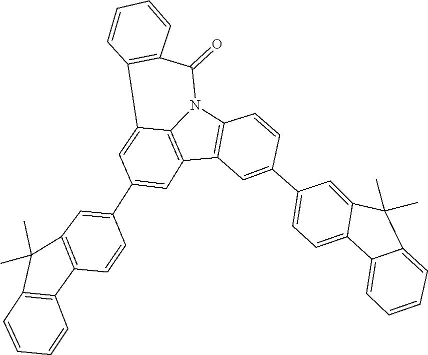 [SMILES: CC1(C)c2ccccc2-c2ccc(-c3ccc4c(c3)c3cc(-c5ccc6c(c5)C(C)(C)c5ccccc5-6)cc5c6ccccc6c(=O)n4c53)cc21]

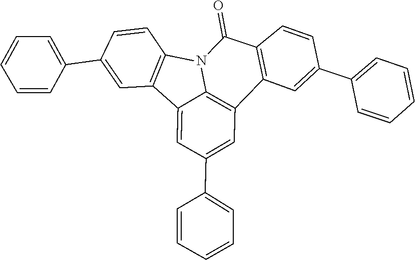 [SMILES: O=c1c2ccc(-c3ccccc3)cc2c2cc(-c3ccccc3)cc3c4cc(-c5ccccc5)ccc4n1c23]

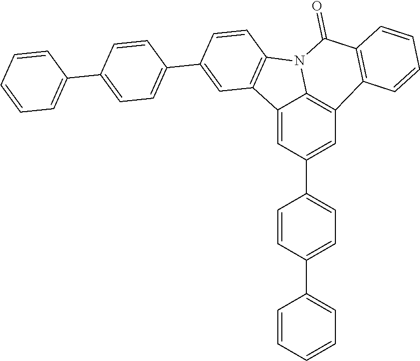 [SMILES: O=c1c2ccccc2c2cc(-c3ccc(-c4ccccc4)cc3)cc3c4cc(-c5ccc(-c6ccccc6)cc5)ccc4n1c23]

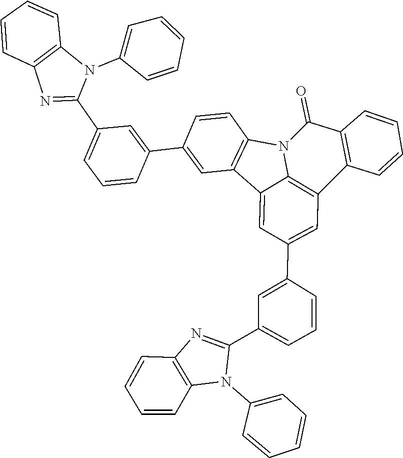 [SMILES: O=c1c2ccccc2c2cc(-c3cccc(-c4nc5ccccc5n4-c4ccccc4)c3)cc3c4cc(-c5cccc(-c6nc7ccccc7n6-c6ccccc6)c5)ccc4n1c23]

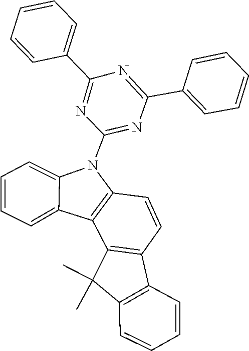 [SMILES: CC1(C)c2ccccc2-c2ccc3c(c21)c1ccccc1n3-c1nc(-c2ccccc2)nc(-c2ccccc2)n1]

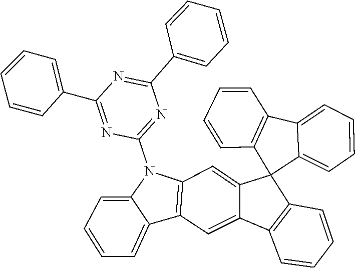 [SMILES: c1ccc(-c2nc(-c3ccccc3)nc(-n3c4ccccc4c4cc5c(cc43)C3(c4ccccc4-c4ccccc43)c3ccccc3-5)n2)cc1]

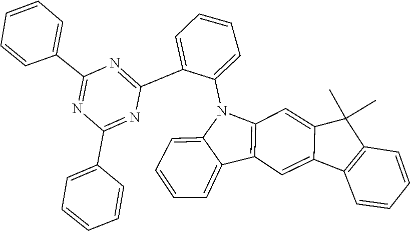 [SMILES: CC1(C)c2ccccc2-c2cc3c4ccccc4n(-c4ccccc4-c4nc(-c5ccccc5)nc(-c5ccccc5)n4)c3cc21]

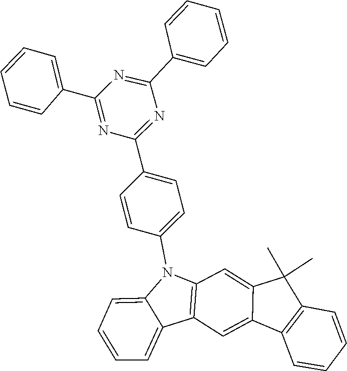 [SMILES: CC1(C)c2ccccc2-c2cc3c4ccccc4n(-c4ccc(-c5nc(-c6ccccc6)nc(-c6ccccc6)n5)cc4)c3cc21]

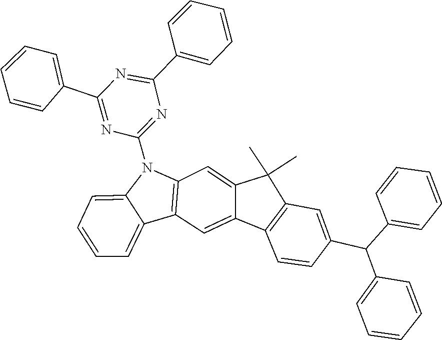 [SMILES: CC1(C)c2cc(C(c3ccccc3)c3ccccc3)ccc2-c2cc3c4ccccc4n(-c4nc(-c5ccccc5)nc(-c5ccccc5)n4)c3cc21]

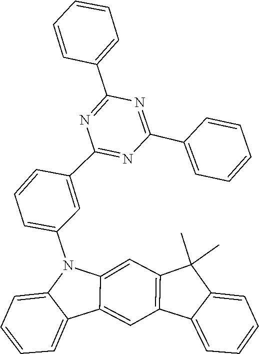 [SMILES: CC1(C)c2ccccc2-c2cc3c4ccccc4n(-c4cccc(-c5nc(-c6ccccc6)nc(-c6ccccc6)n5)c4)c3cc21]

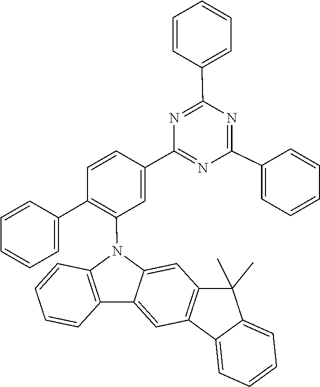 [SMILES: CC1(C)c2ccccc2-c2cc3c4ccccc4n(-c4cc(-c5nc(-c6ccccc6)nc(-c6ccccc6)n5)ccc4-c4ccccc4)c3cc21]

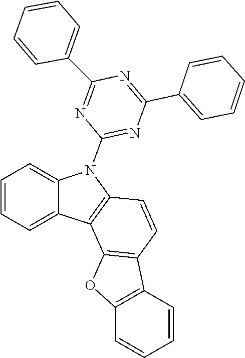 [SMILES: c1ccc(-c2nc(-c3ccccc3)nc(-n3c4ccccc4c4c5oc6ccccc6c5ccc43)n2)cc1]

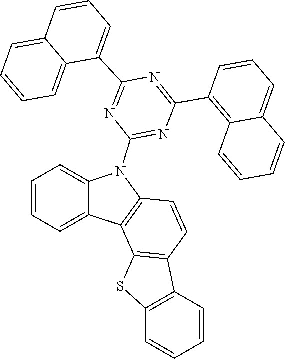 [SMILES: c1ccc2c(-c3nc(-c4cccc5ccccc45)nc(-n4c5ccccc5c5c6sc7ccccc7c6ccc54)n3)cccc2c1]

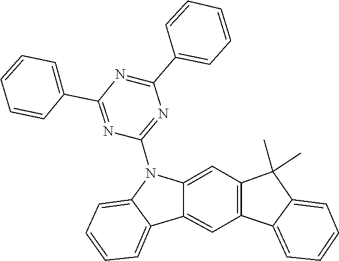 [SMILES: CC1(C)c2ccccc2-c2cc3c4ccccc4n(-c4nc(-c5ccccc5)nc(-c5ccccc5)n4)c3cc21]

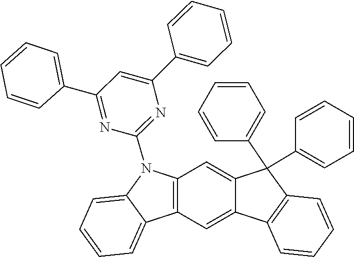 [SMILES: c1ccc(-c2cc(-c3ccccc3)nc(-n3c4ccccc4c4cc5c(cc43)C(c3ccccc3)(c3ccccc3)c3ccccc3-5)n2)cc1]

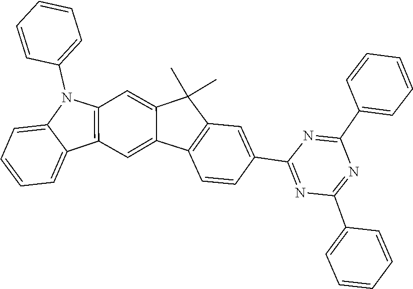 [SMILES: CC1(C)c2cc(-c3nc(-c4ccccc4)nc(-c4ccccc4)n3)ccc2-c2cc3c4ccccc4n(-c4ccccc4)c3cc21]

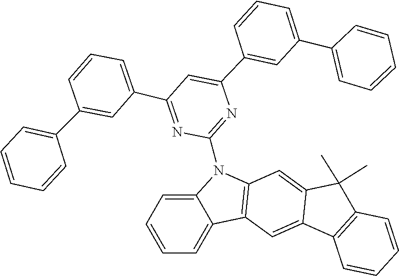 [SMILES: CC1(C)c2ccccc2-c2cc3c4ccccc4n(-c4nc(-c5cccc(-c6ccccc6)c5)cc(-c5cccc(-c6ccccc6)c5)n4)c3cc21]

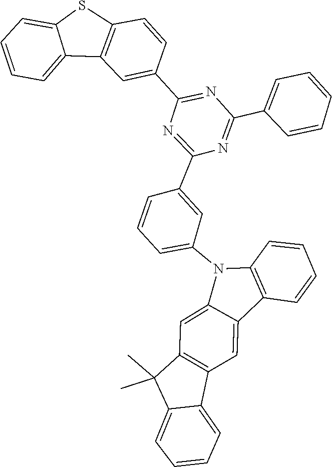 [SMILES: CC1(C)c2ccccc2-c2cc3c4ccccc4n(-c4cccc(-c5nc(-c6ccccc6)nc(-c6ccc7sc8ccccc8c7c6)n5)c4)c3cc21]

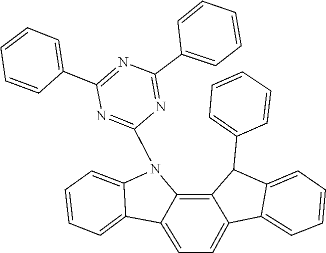 [SMILES: c1ccc(-c2nc(-c3ccccc3)nc(-n3c4ccccc4c4ccc5c(c43)C(c3ccccc3)c3ccccc3-5)n2)cc1]

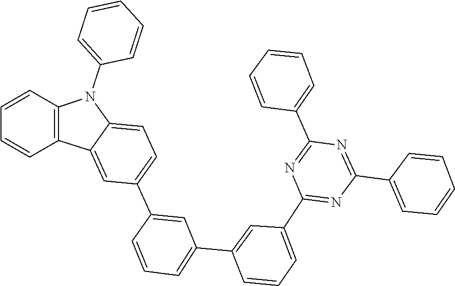 [SMILES: c1ccc(-c2nc(-c3ccccc3)nc(-c3cccc(-c4cccc(-c5ccc6c(c5)c5ccccc5n6-c5ccccc5)c4)c3)n2)cc1]

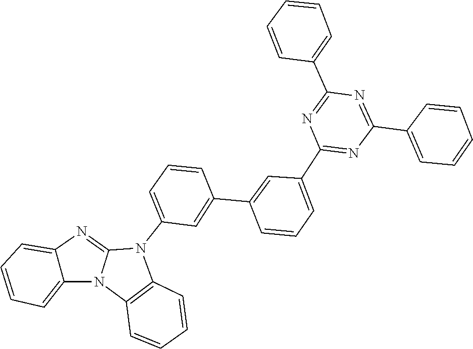 [SMILES: c1ccc(-c2nc(-c3ccccc3)nc(-c3cccc(-c4cccc(-n5c6ccccc6n6c7ccccc7nc56)c4)c3)n2)cc1]

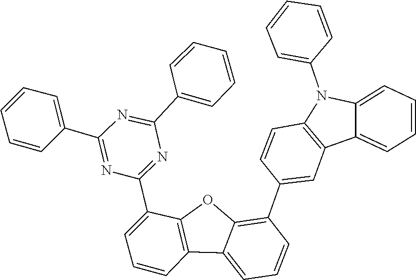 [SMILES: c1ccc(-c2nc(-c3ccccc3)nc(-c3cccc4c3oc3c(-c5ccc6c(c5)c5ccccc5n6-c5ccccc5)cccc34)n2)cc1]

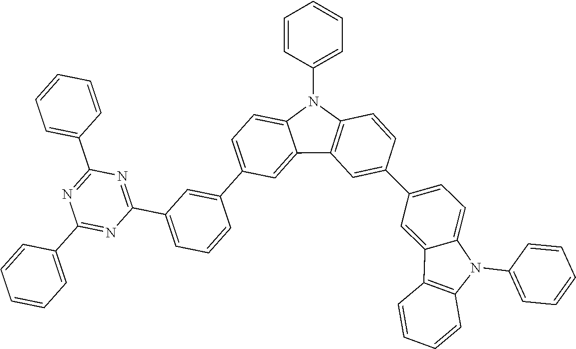 [SMILES: c1ccc(-c2nc(-c3ccccc3)nc(-c3cccc(-c4ccc5c(c4)c4cc(-c6ccc7c(c6)c6ccccc6n7-c6ccccc6)ccc4n5-c4ccccc4)c3)n2)cc1]

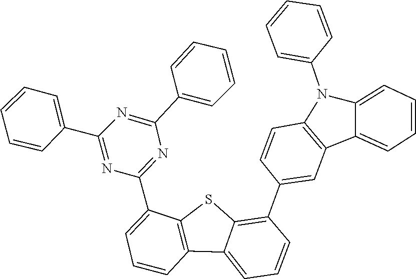 [SMILES: c1ccc(-c2nc(-c3ccccc3)nc(-c3cccc4c3sc3c(-c5ccc6c(c5)c5ccccc5n6-c5ccccc5)cccc34)n2)cc1]

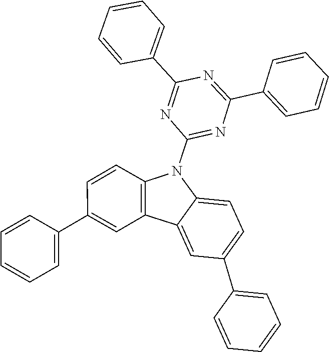 [SMILES: c1ccc(-c2ccc3c(c2)c2cc(-c4ccccc4)ccc2n3-c2nc(-c3ccccc3)nc(-c3ccccc3)n2)cc1]

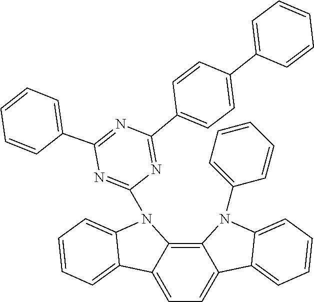 [SMILES: c1ccc(-c2ccc(-c3nc(-c4ccccc4)nc(-n4c5ccccc5c5ccc6c7ccccc7n(-c7ccccc7)c6c54)n3)cc2)cc1]

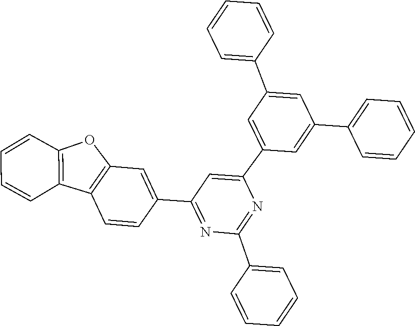 [SMILES: c1ccc(-c2cc(-c3ccccc3)cc(-c3cc(-c4ccc5c(c4)oc4ccccc45)nc(-c4ccccc4)n3)c2)cc1]

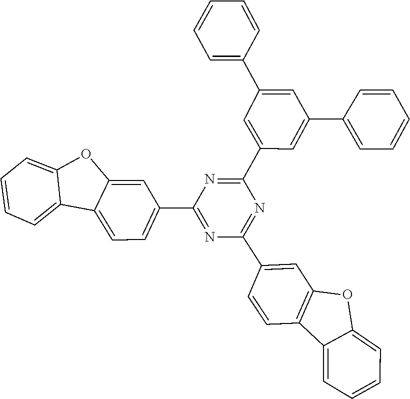 [SMILES: c1ccc(-c2cc(-c3ccccc3)cc(-c3nc(-c4ccc5c(c4)oc4ccccc45)nc(-c4ccc5c(c4)oc4ccccc45)n3)c2)cc1]

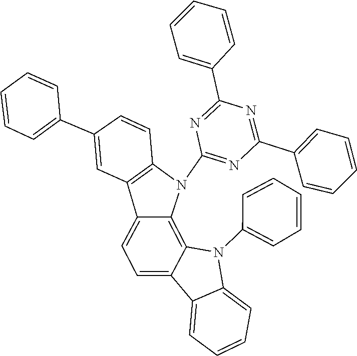 [SMILES: c1ccc(-c2ccc3c(c2)c2ccc4c5ccccc5n(-c5ccccc5)c4c2n3-c2nc(-c3ccccc3)nc(-c3ccccc3)n2)cc1]

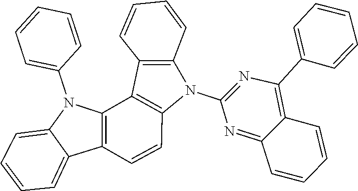 [SMILES: c1ccc(-c2nc(-n3c4ccccc4c4c3ccc3c5ccccc5n(-c5ccccc5)c34)nc3ccccc23)cc1]

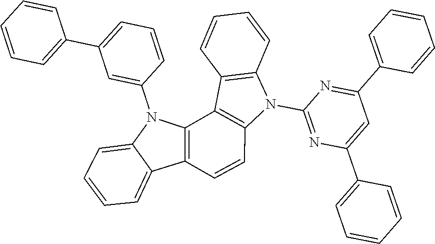 [SMILES: c1ccc(-c2cccc(-n3c4ccccc4c4ccc5c(c6ccccc6n5-c5nc(-c6ccccc6)cc(-c6ccccc6)n5)c43)c2)cc1]

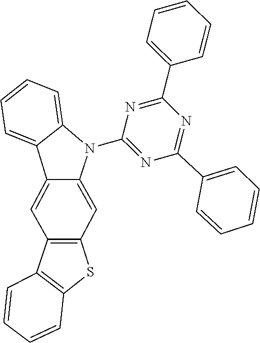 [SMILES: c1ccc(-c2nc(-c3ccccc3)nc(-n3c4ccccc4c4cc5c(cc43)sc3ccccc35)n2)cc1]

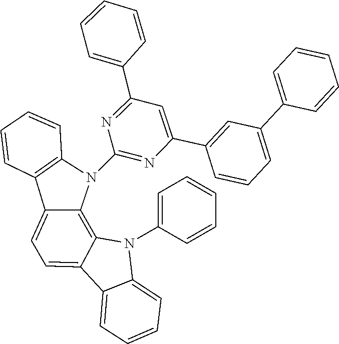 [SMILES: c1ccc(-c2cccc(-c3cc(-c4ccccc4)nc(-n4c5ccccc5c5ccc6c7ccccc7n(-c7ccccc7)c6c54)n3)c2)cc1]

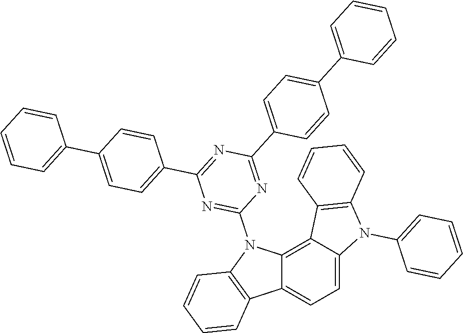 [SMILES: c1ccc(-c2ccc(-c3nc(-c4ccc(-c5ccccc5)cc4)nc(-n4c5ccccc5c5ccc6c(c7ccccc7n6-c6ccccc6)c54)n3)cc2)cc1]

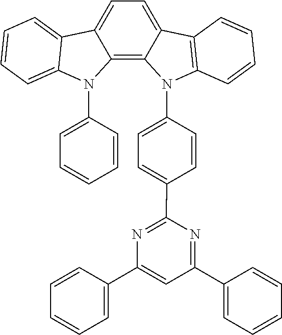 [SMILES: c1ccc(-c2cc(-c3ccccc3)nc(-c3ccc(-n4c5ccccc5c5ccc6c7ccccc7n(-c7ccccc7)c6c54)cc3)n2)cc1]

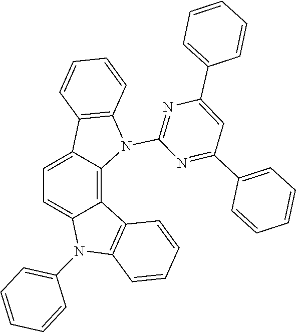 [SMILES: c1ccc(-c2cc(-c3ccccc3)nc(-n3c4ccccc4c4ccc5c(c6ccccc6n5-c5ccccc5)c43)n2)cc1]

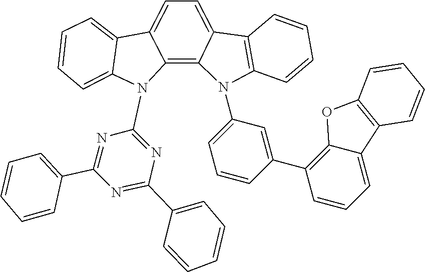 [SMILES: c1ccc(-c2nc(-c3ccccc3)nc(-n3c4ccccc4c4ccc5c6ccccc6n(-c6cccc(-c7cccc8c7oc7ccccc78)c6)c5c43)n2)cc1]

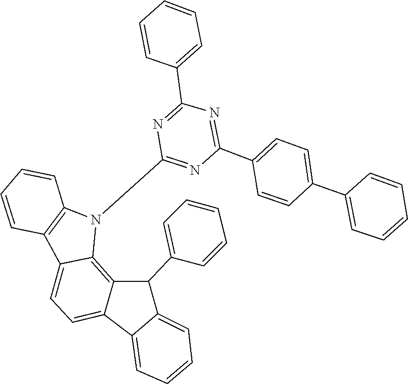 [SMILES: c1ccc(-c2ccc(-c3nc(-c4ccccc4)nc(-n4c5ccccc5c5ccc6c(c54)C(c4ccccc4)c4ccccc4-6)n3)cc2)cc1]

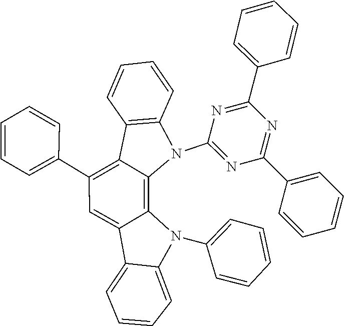 [SMILES: c1ccc(-c2nc(-c3ccccc3)nc(-n3c4ccccc4c4c(-c5ccccc5)cc5c6ccccc6n(-c6ccccc6)c5c43)n2)cc1]

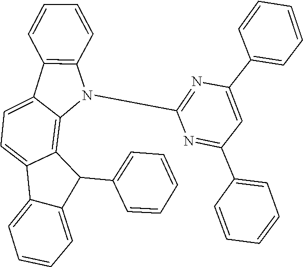 [SMILES: c1ccc(-c2cc(-c3ccccc3)nc(-n3c4ccccc4c4ccc5c(c43)C(c3ccccc3)c3ccccc3-5)n2)cc1]

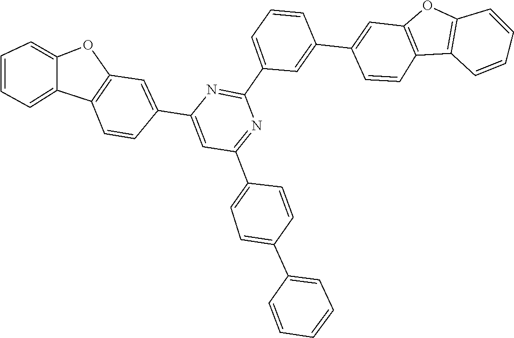 [SMILES: c1ccc(-c2ccc(-c3cc(-c4ccc5c(c4)oc4ccccc45)nc(-c4cccc(-c5ccc6c(c5)oc5ccccc56)c4)n3)cc2)cc1]

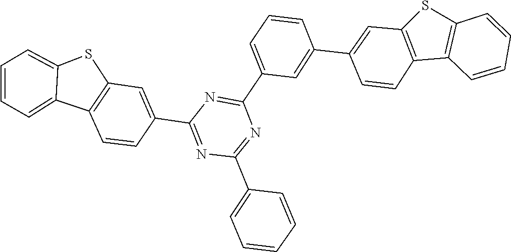 [SMILES: c1ccc(-c2nc(-c3cccc(-c4ccc5c(c4)sc4ccccc45)c3)nc(-c3ccc4c(c3)sc3ccccc34)n2)cc1]

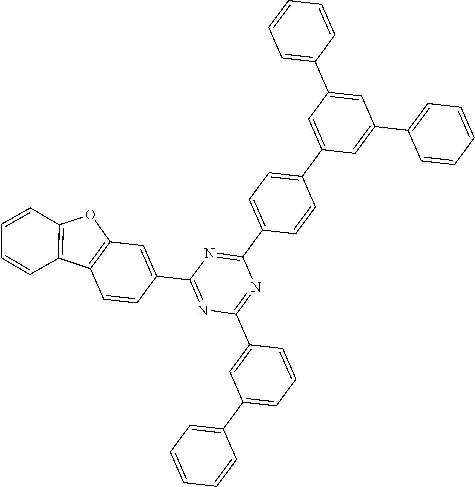 [SMILES: c1ccc(-c2cc(-c3ccccc3)cc(-c3ccc(-c4nc(-c5cccc(-c6ccccc6)c5)nc(-c5ccc6c(c5)oc5ccccc56)n4)cc3)c2)cc1]

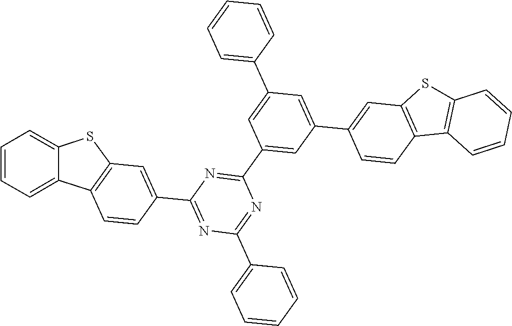 [SMILES: c1ccc(-c2cc(-c3ccc4c(c3)sc3ccccc34)cc(-c3nc(-c4ccccc4)nc(-c4ccc5c(c4)sc4ccccc45)n3)c2)cc1]

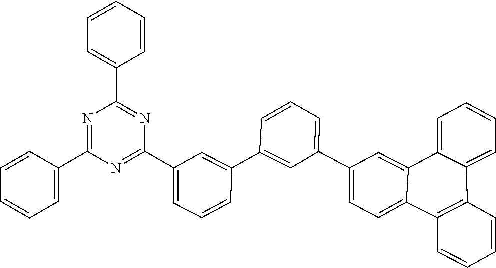 [SMILES: c1ccc(-c2nc(-c3ccccc3)nc(-c3cccc(-c4cccc(-c5ccc6c7ccccc7c7ccccc7c6c5)c4)c3)n2)cc1]

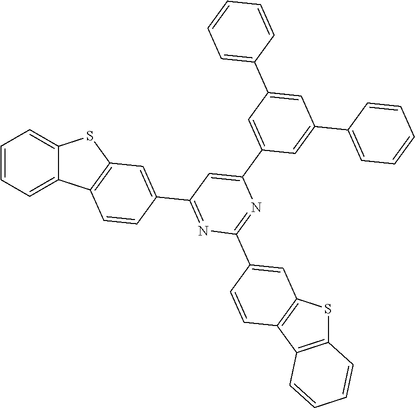 [SMILES: c1ccc(-c2cc(-c3ccccc3)cc(-c3cc(-c4ccc5c(c4)sc4ccccc45)nc(-c4ccc5c(c4)sc4ccccc45)n3)c2)cc1]

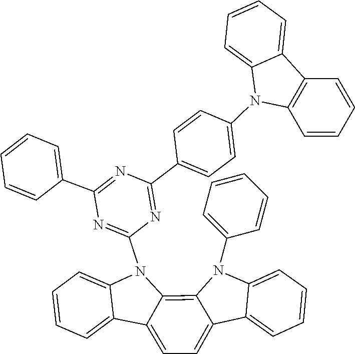 [SMILES: c1ccc(-c2nc(-c3ccc(-n4c5ccccc5c5ccccc54)cc3)nc(-n3c4ccccc4c4ccc5c6ccccc6n(-c6ccccc6)c5c43)n2)cc1]

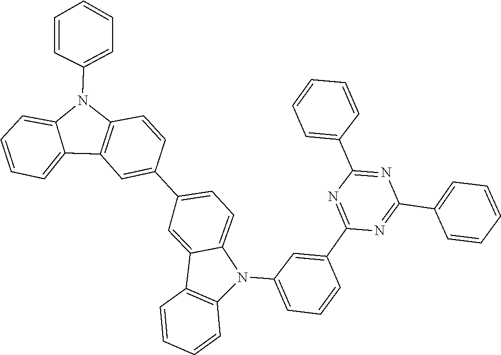 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

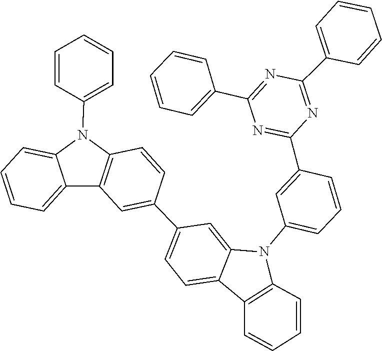 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1]

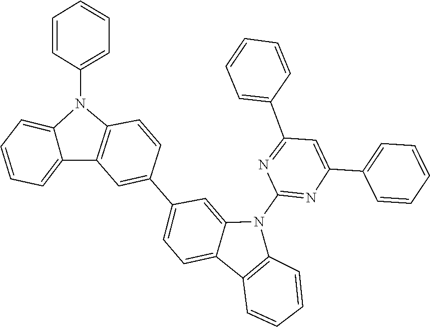 [SMILES: c1ccc(-c2cc(-c3ccccc3)nc(-n3c4ccccc4c4ccc(-c5ccc6c(c5)c5ccccc5n6-c5ccccc5)cc43)n2)cc1]

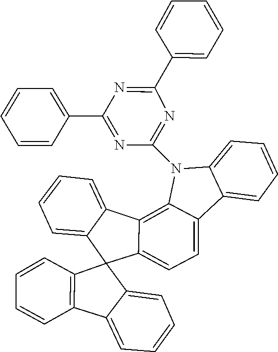 [SMILES: c1ccc(-c2nc(-c3ccccc3)nc(-n3c4ccccc4c4ccc5c(c43)-c3ccccc3C53c4ccccc4-c4ccccc43)n2)cc1]

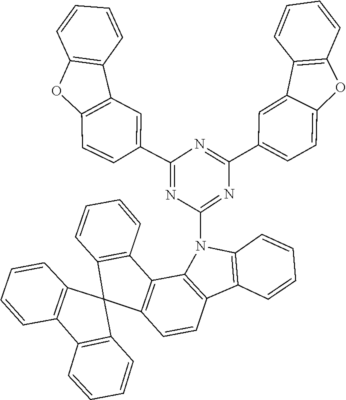 [SMILES: c1ccc2c(c1)-c1ccccc1C21c2ccccc2-c2c1ccc1c3ccccc3n(-c3nc(-c4ccc5oc6ccccc6c5c4)nc(-c4ccc5oc6ccccc6c5c4)n3)c21]

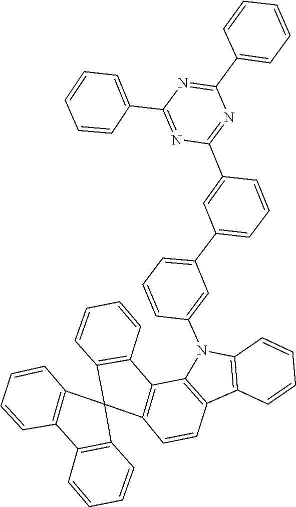 [SMILES: c1ccc(-c2nc(-c3ccccc3)nc(-c3cccc(-c4cccc(-n5c6ccccc6c6ccc7c(c65)-c5ccccc5C75c6ccccc6-c6ccccc65)c4)c3)n2)cc1]

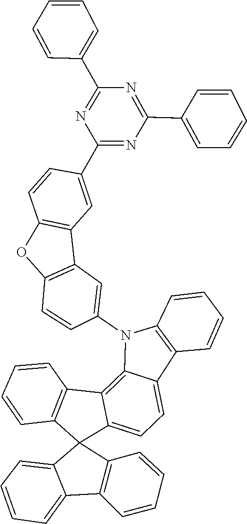 [SMILES: c1ccc(-c2nc(-c3ccccc3)nc(-c3ccc4oc5ccc(-n6c7ccccc7c7ccc8c(c76)-c6ccccc6C86c7ccccc7-c7ccccc76)cc5c4c3)n2)cc1]

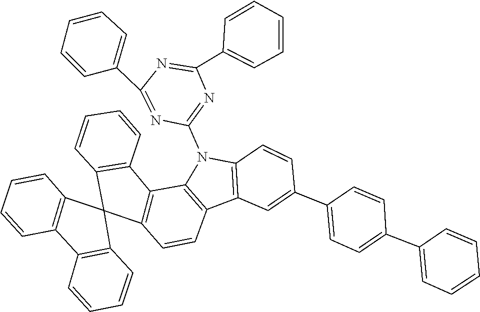 [SMILES: c1ccc(-c2ccc(-c3ccc4c(c3)c3ccc5c(c3n4-c3nc(-c4ccccc4)nc(-c4ccccc4)n3)-c3ccccc3C53c4ccccc4-c4ccccc43)cc2)cc1]

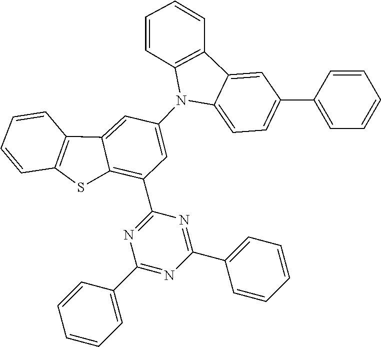 [SMILES: c1ccc(-c2ccc3c(c2)c2ccccc2n3-c2cc(-c3nc(-c4ccccc4)nc(-c4ccccc4)n3)c3sc4ccccc4c3c2)cc1]

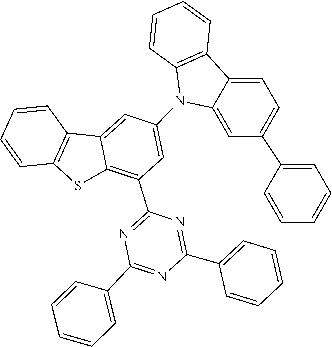 [SMILES: c1ccc(-c2ccc3c4ccccc4n(-c4cc(-c5nc(-c6ccccc6)nc(-c6ccccc6)n5)c5sc6ccccc6c5c4)c3c2)cc1]

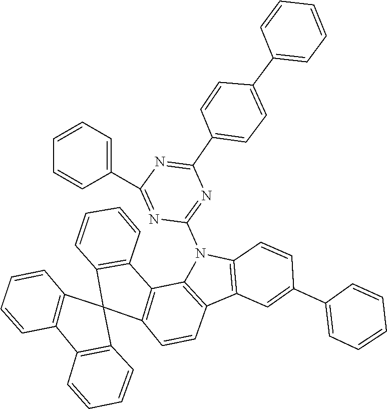 [SMILES: c1ccc(-c2ccc(-c3nc(-c4ccccc4)nc(-n4c5ccc(-c6ccccc6)cc5c5ccc6c(c54)-c4ccccc4C64c5ccccc5-c5ccccc54)n3)cc2)cc1]

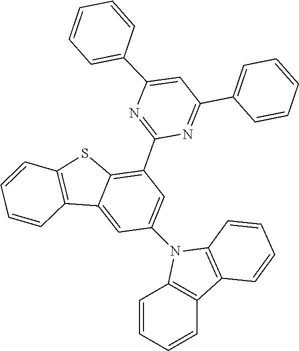 [SMILES: c1ccc(-c2cc(-c3ccccc3)nc(-c3cc(-n4c5ccccc5c5ccccc54)cc4c3sc3ccccc34)n2)cc1]

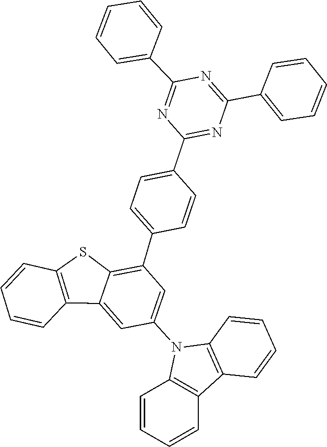 [SMILES: c1ccc(-c2nc(-c3ccccc3)nc(-c3ccc(-c4cc(-n5c6ccccc6c6ccccc65)cc5c4sc4ccccc45)cc3)n2)cc1]

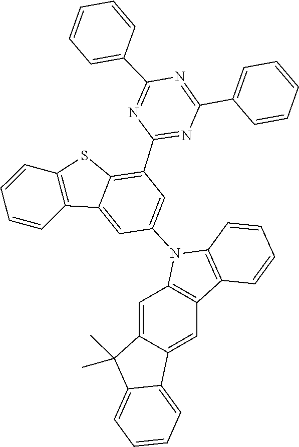 [SMILES: CC1(C)c2ccccc2-c2cc3c4ccccc4n(-c4cc(-c5nc(-c6ccccc6)nc(-c6ccccc6)n5)c5sc6ccccc6c5c4)c3cc21]

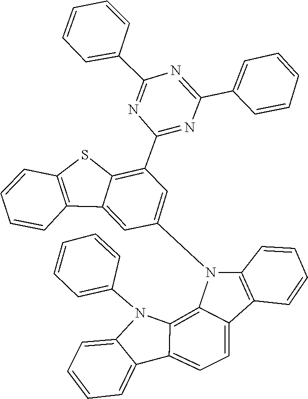 [SMILES: c1ccc(-c2nc(-c3ccccc3)nc(-c3cc(-n4c5ccccc5c5ccc6c7ccccc7n(-c7ccccc7)c6c54)cc4c3sc3ccccc34)n2)cc1]

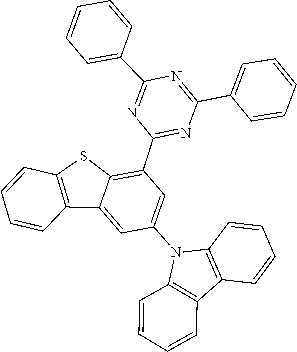 [SMILES: c1ccc(-c2nc(-c3ccccc3)nc(-c3cc(-n4c5ccccc5c5ccccc54)cc4c3sc3ccccc34)n2)cc1]

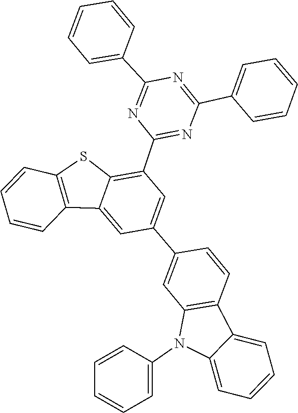 [SMILES: c1ccc(-c2nc(-c3ccccc3)nc(-c3cc(-c4ccc5c6ccccc6n(-c6ccccc6)c5c4)cc4c3sc3ccccc34)n2)cc1]

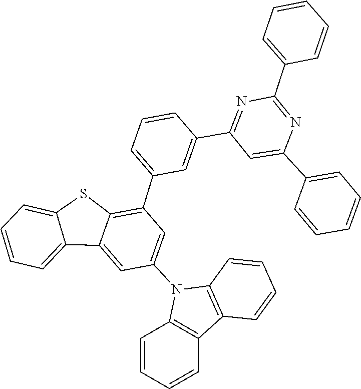 [SMILES: c1ccc(-c2cc(-c3cccc(-c4cc(-n5c6ccccc6c6ccccc65)cc5c4sc4ccccc45)c3)nc(-c3ccccc3)n2)cc1]

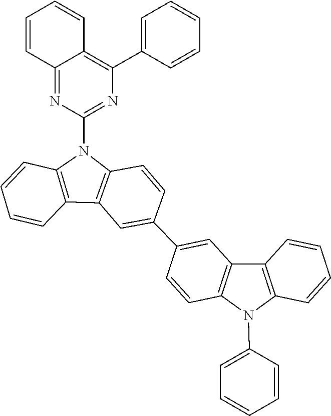 [SMILES: c1ccc(-c2nc(-n3c4ccccc4c4cc(-c5ccc6c(c5)c5ccccc5n6-c5ccccc5)ccc43)nc3ccccc23)cc1]

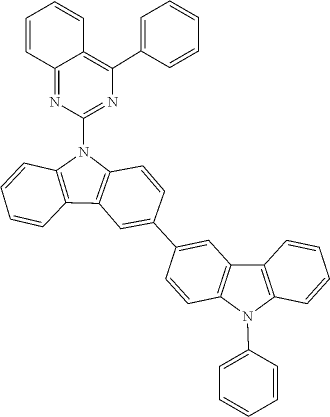 [SMILES: c1ccc(-c2nc(-n3c4ccccc4c4cc(-c5ccc6c(c5)c5ccccc5n6-c5ccccc5)ccc43)nc3ccccc23)cc1]

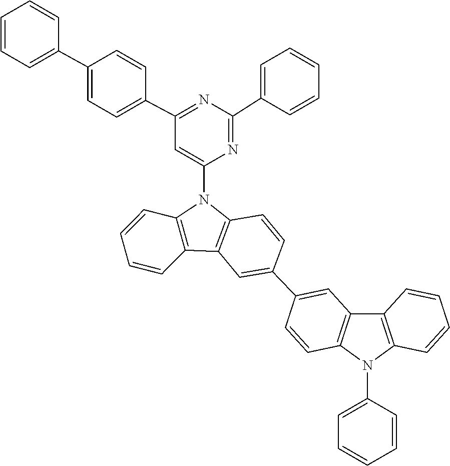 [SMILES: c1ccc(-c2ccc(-c3cc(-n4c5ccccc5c5cc(-c6ccc7c(c6)c6ccccc6n7-c6ccccc6)ccc54)nc(-c4ccccc4)n3)cc2)cc1]

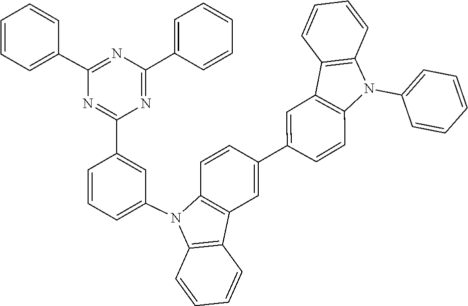 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

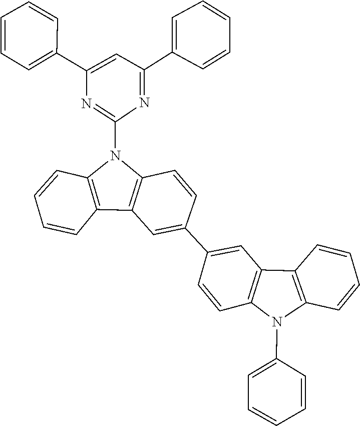 [SMILES: c1ccc(-c2cc(-c3ccccc3)nc(-n3c4ccccc4c4cc(-c5ccc6c(c5)c5ccccc5n6-c5ccccc5)ccc43)n2)cc1]

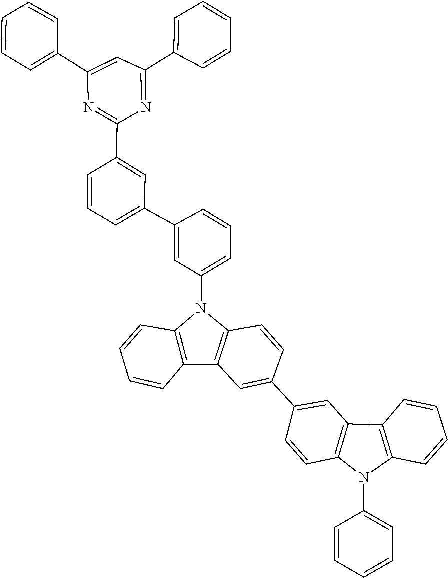 [SMILES: c1ccc(-c2cc(-c3ccccc3)nc(-c3cccc(-c4cccc(-n5c6ccccc6c6cc(-c7ccc8c(c7)c7ccccc7n8-c7ccccc7)ccc65)c4)c3)n2)cc1]

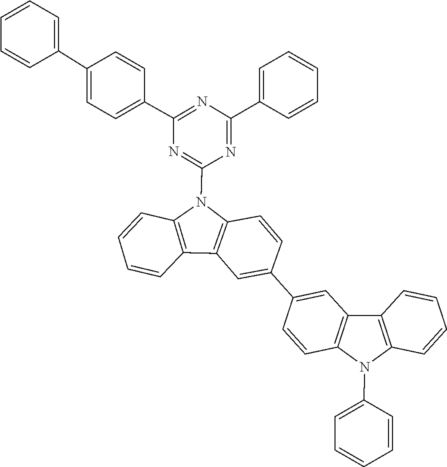 [SMILES: c1ccc(-c2ccc(-c3nc(-c4ccccc4)nc(-n4c5ccccc5c5cc(-c6ccc7c(c6)c6ccccc6n7-c6ccccc6)ccc54)n3)cc2)cc1]

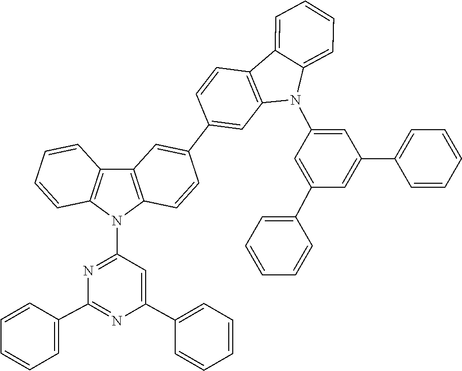 [SMILES: c1ccc(-c2cc(-c3ccccc3)cc(-n3c4ccccc4c4ccc(-c5ccc6c(c5)c5ccccc5n6-c5cc(-c6ccccc6)nc(-c6ccccc6)n5)cc43)c2)cc1]

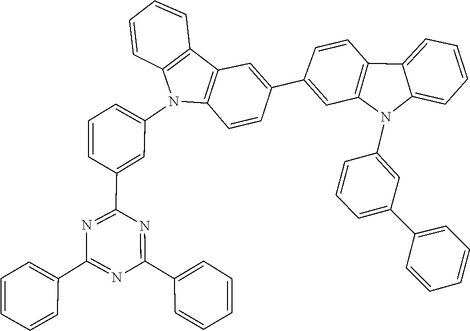 [SMILES: c1ccc(-c2cccc(-n3c4ccccc4c4ccc(-c5ccc6c(c5)c5ccccc5n6-c5cccc(-c6nc(-c7ccccc7)nc(-c7ccccc7)n6)c5)cc43)c2)cc1]

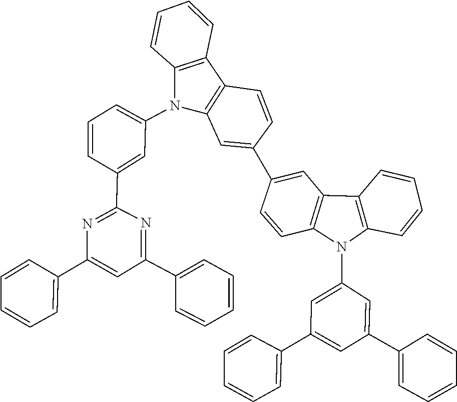 [SMILES: c1ccc(-c2cc(-c3ccccc3)cc(-n3c4ccccc4c4cc(-c5ccc6c7ccccc7n(-c7cccc(-c8nc(-c9ccccc9)cc(-c9ccccc9)n8)c7)c6c5)ccc43)c2)cc1]

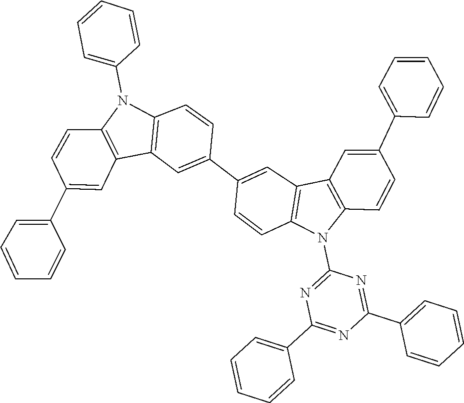 [SMILES: c1ccc(-c2ccc3c(c2)c2cc(-c4ccc5c(c4)c4cc(-c6ccccc6)ccc4n5-c4nc(-c5ccccc5)nc(-c5ccccc5)n4)ccc2n3-c2ccccc2)cc1]

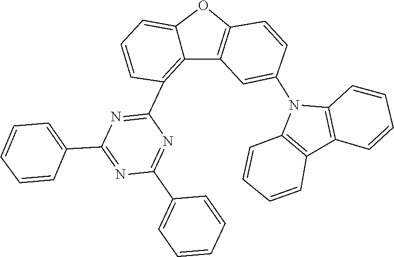 [SMILES: c1ccc(-c2nc(-c3ccccc3)nc(-c3cccc4oc5ccc(-n6c7ccccc7c7ccccc76)cc5c34)n2)cc1]

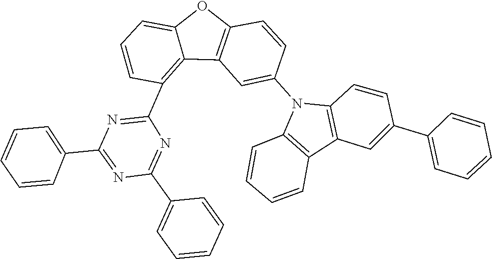 [SMILES: c1ccc(-c2ccc3c(c2)c2ccccc2n3-c2ccc3oc4cccc(-c5nc(-c6ccccc6)nc(-c6ccccc6)n5)c4c3c2)cc1]

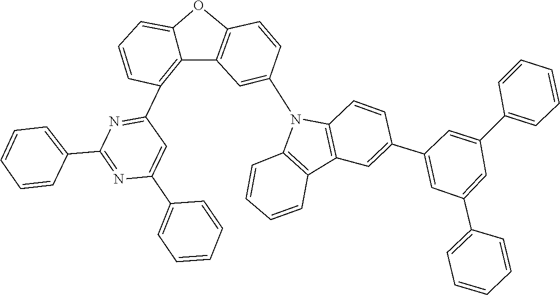 [SMILES: c1ccc(-c2cc(-c3ccccc3)cc(-c3ccc4c(c3)c3ccccc3n4-c3ccc4oc5cccc(-c6cc(-c7ccccc7)nc(-c7ccccc7)n6)c5c4c3)c2)cc1]

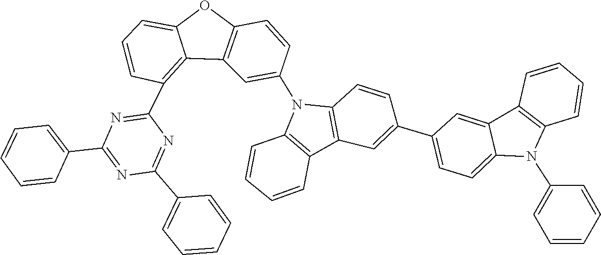 [SMILES: c1ccc(-c2nc(-c3ccccc3)nc(-c3cccc4oc5ccc(-n6c7ccccc7c7cc(-c8ccc9c(c8)c8ccccc8n9-c8ccccc8)ccc76)cc5c34)n2)cc1]

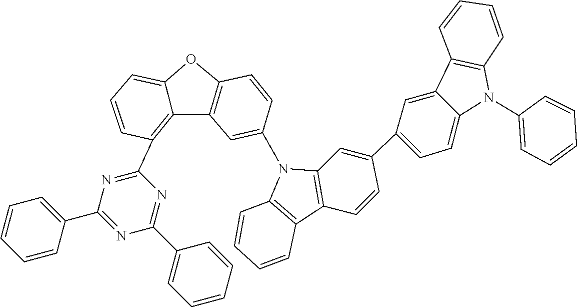 [SMILES: c1ccc(-c2nc(-c3ccccc3)nc(-c3cccc4oc5ccc(-n6c7ccccc7c7ccc(-c8ccc9c(c8)c8ccccc8n9-c8ccccc8)cc76)cc5c34)n2)cc1]

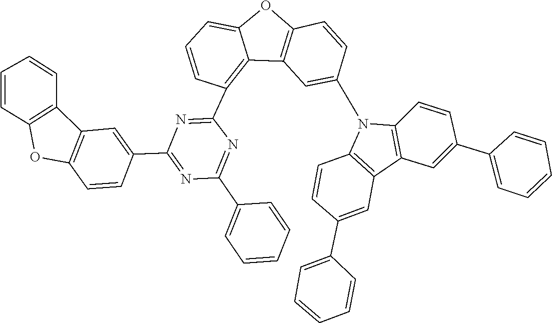 [SMILES: c1ccc(-c2ccc3c(c2)c2cc(-c4ccccc4)ccc2n3-c2ccc3oc4cccc(-c5nc(-c6ccccc6)nc(-c6ccc7oc8ccccc8c7c6)n5)c4c3c2)cc1]

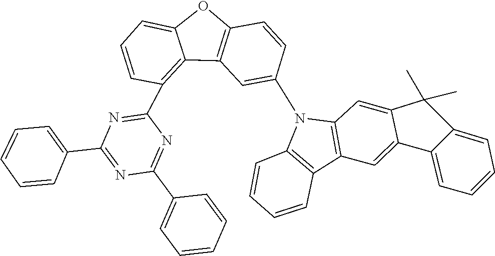 [SMILES: CC1(C)c2ccccc2-c2cc3c4ccccc4n(-c4ccc5oc6cccc(-c7nc(-c8ccccc8)nc(-c8ccccc8)n7)c6c5c4)c3cc21]

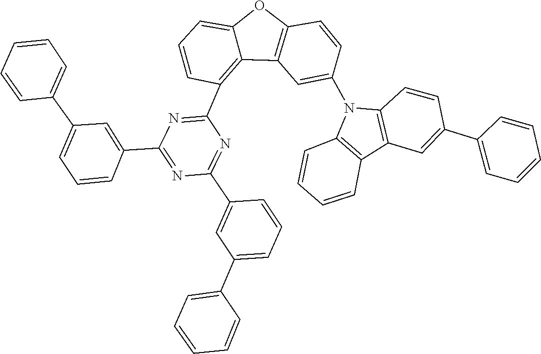 [SMILES: c1ccc(-c2cccc(-c3nc(-c4cccc(-c5ccccc5)c4)nc(-c4cccc5oc6ccc(-n7c8ccccc8c8cc(-c9ccccc9)ccc87)cc6c45)n3)c2)cc1]

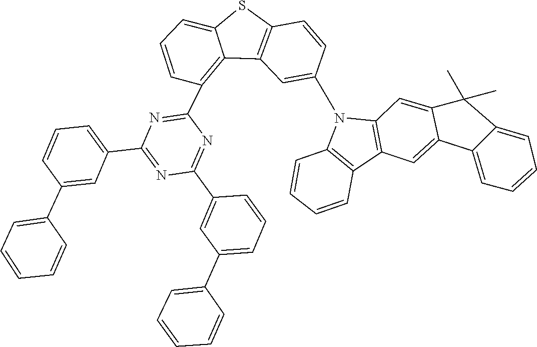 [SMILES: CC1(C)c2ccccc2-c2cc3c4ccccc4n(-c4ccc5sc6cccc(-c7nc(-c8cccc(-c9ccccc9)c8)nc(-c8cccc(-c9ccccc9)c8)n7)c6c5c4)c3cc21]

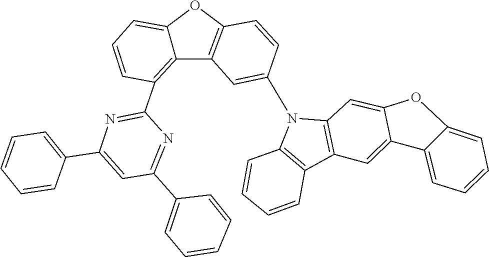 [SMILES: c1ccc(-c2cc(-c3ccccc3)nc(-c3cccc4oc5ccc(-n6c7ccccc7c7cc8c(cc76)oc6ccccc68)cc5c34)n2)cc1]

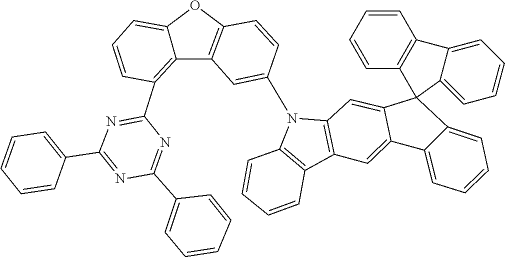 [SMILES: c1ccc(-c2nc(-c3ccccc3)nc(-c3cccc4oc5ccc(-n6c7ccccc7c7cc8c(cc76)C6(c7ccccc7-c7ccccc76)c6ccccc6-8)cc5c34)n2)cc1]

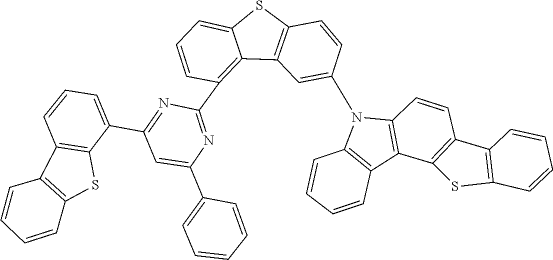 [SMILES: c1ccc(-c2cc(-c3cccc4c3sc3ccccc34)nc(-c3cccc4sc5ccc(-n6c7ccccc7c7c8sc9ccccc9c8ccc76)cc5c34)n2)cc1]

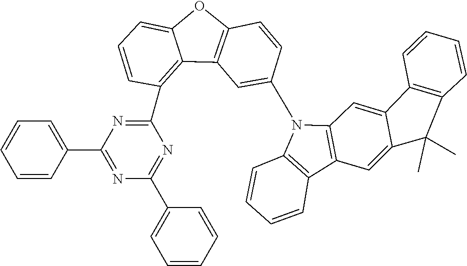 [SMILES: CC1(C)c2ccccc2-c2cc3c(cc21)c1ccccc1n3-c1ccc2oc3cccc(-c4nc(-c5ccccc5)nc(-c5ccccc5)n4)c3c2c1]

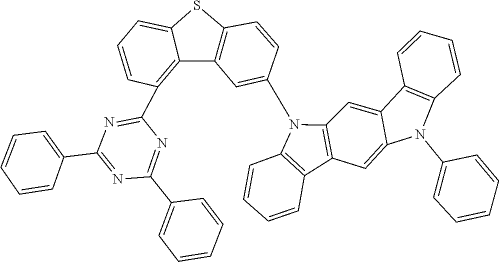 [SMILES: c1ccc(-c2nc(-c3ccccc3)nc(-c3cccc4sc5ccc(-n6c7ccccc7c7cc8c(cc76)c6ccccc6n8-c6ccccc6)cc5c34)n2)cc1]

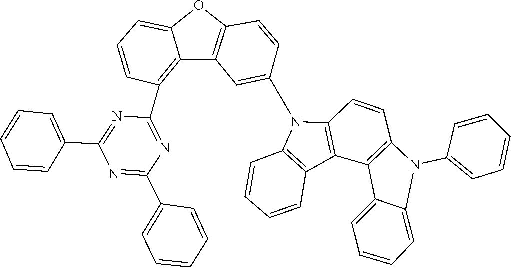 [SMILES: c1ccc(-c2nc(-c3ccccc3)nc(-c3cccc4oc5ccc(-n6c7ccccc7c7c8c9ccccc9n(-c9ccccc9)c8ccc76)cc5c34)n2)cc1]

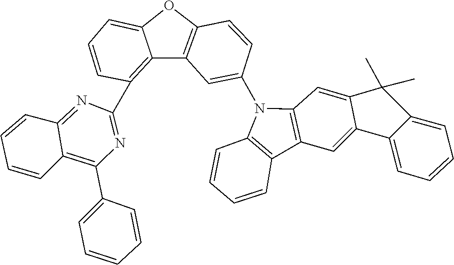 [SMILES: CC1(C)c2ccccc2-c2cc3c4ccccc4n(-c4ccc5oc6cccc(-c7nc(-c8ccccc8)c8ccccc8n7)c6c5c4)c3cc21]

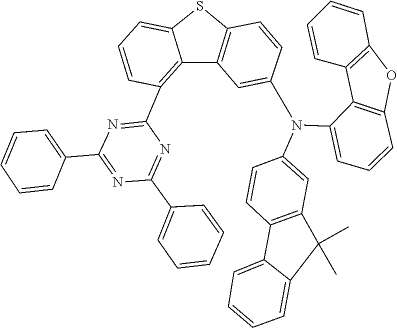 [SMILES: CC1(C)c2ccccc2-c2ccc(N(c3ccc4sc5cccc(-c6nc(-c7ccccc7)nc(-c7ccccc7)n6)c5c4c3)c3cccc4oc5ccccc5c34)cc21]

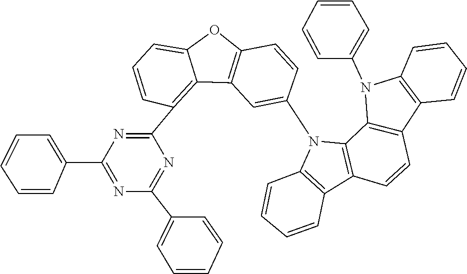 [SMILES: c1ccc(-c2nc(-c3ccccc3)nc(-c3cccc4oc5ccc(-n6c7ccccc7c7ccc8c9ccccc9n(-c9ccccc9)c8c76)cc5c34)n2)cc1]

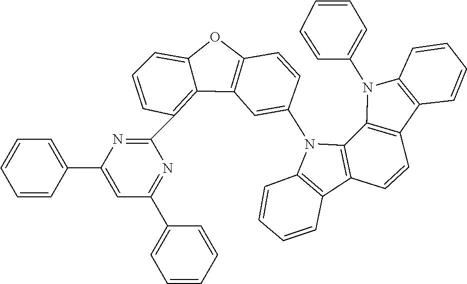 [SMILES: c1ccc(-c2cc(-c3ccccc3)nc(-c3cccc4oc5ccc(-n6c7ccccc7c7ccc8c9ccccc9n(-c9ccccc9)c8c76)cc5c34)n2)cc1]

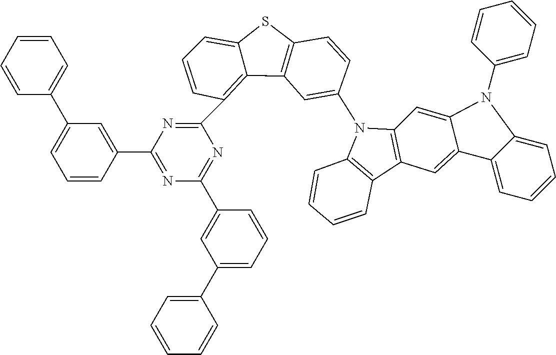 [SMILES: c1ccc(-c2cccc(-c3nc(-c4cccc(-c5ccccc5)c4)nc(-c4cccc5sc6ccc(-n7c8ccccc8c8cc9c%10ccccc%10n(-c%10ccccc%10)c9cc87)cc6c45)n3)c2)cc1]

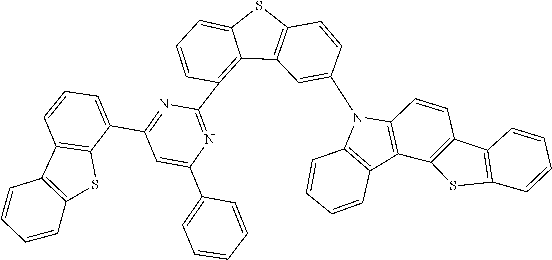 [SMILES: c1ccc(-c2cc(-c3cccc4c3sc3ccccc34)nc(-c3cccc4sc5ccc(-n6c7ccccc7c7c8sc9ccccc9c8ccc76)cc5c34)n2)cc1]

The table that follows shows examples of suitable hole-transporting matrix materials that can be used together with the materials of the invention.

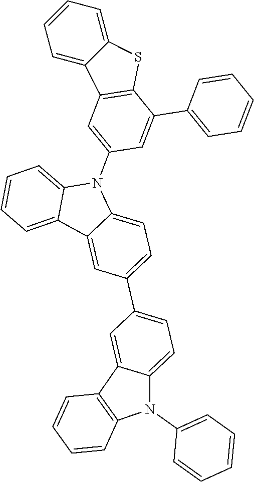 [SMILES: c1ccc(-c2cc(-n3c4ccccc4c4cc(-c5ccc6c(c5)c5ccccc5n6-c5ccccc5)ccc43)cc3c2sc2ccccc23)cc1]

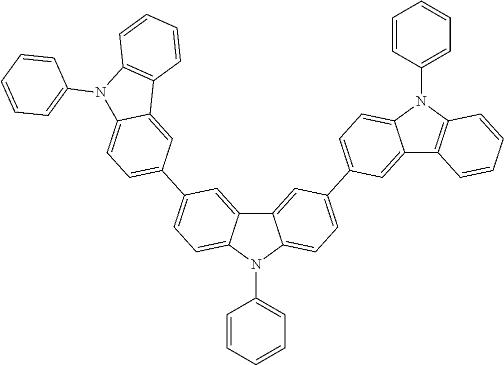 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

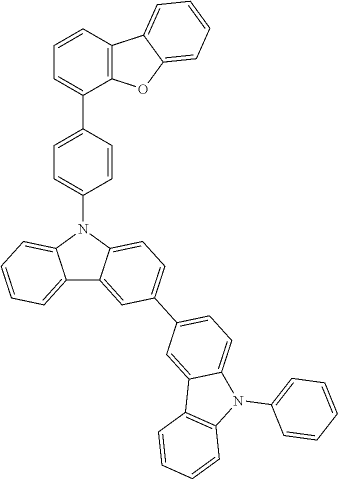 [SMILES: c1ccc(-n2c3ccccc3c3cc(-c4ccc5c(c4)c4ccccc4n5-c4ccc(-c5cccc6c5oc5ccccc56)cc4)ccc32)cc1]

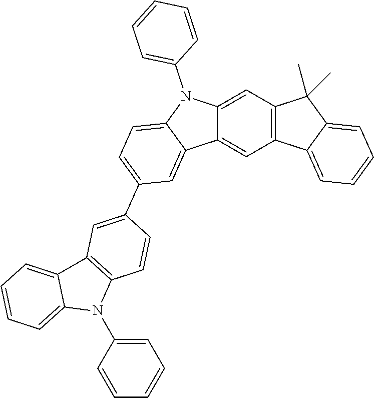 [SMILES: CC1(C)c2ccccc2-c2cc3c4cc(-c5ccc6c(c5)c5ccccc5n6-c5ccccc5)ccc4n(-c4ccccc4)c3cc21]

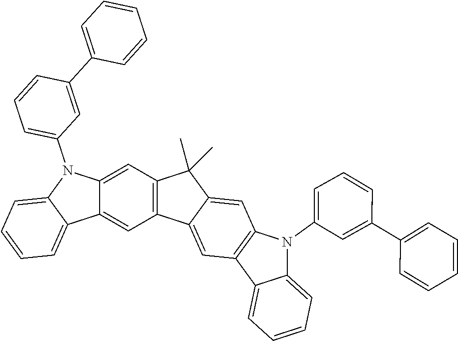 [SMILES: CC1(C)c2cc3c(cc2-c2cc4c5ccccc5n(-c5cccc(-c6ccccc6)c5)c4cc21)c1ccccc1n3-c1cccc(-c2ccccc2)c1]

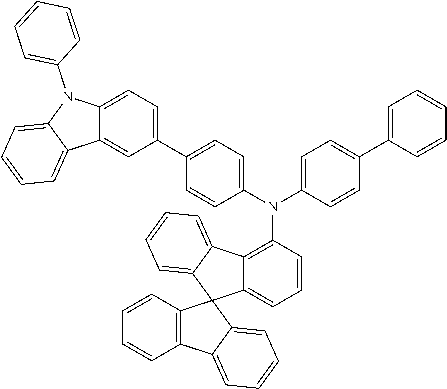 [SMILES: c1ccc(-c2ccc(N(c3ccc(-c4ccc5c(c4)c4ccccc4n5-c4ccccc4)cc3)c3cccc4c3-c3ccccc3C43c4ccccc4-c4ccccc43)cc2)cc1]

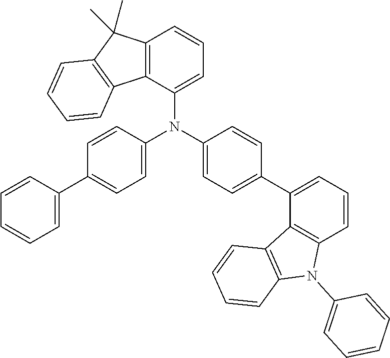 [SMILES: CC1(C)c2ccccc2-c2c(N(c3ccc(-c4ccccc4)cc3)c3ccc(-c4cccc5c4c4ccccc4n5-c4ccccc4)cc3)cccc21]

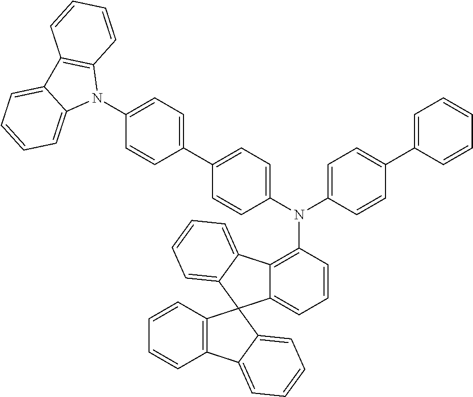 [SMILES: c1ccc(-c2ccc(N(c3ccc(-c4ccc(-n5c6ccccc6c6ccccc65)cc4)cc3)c3cccc4c3-c3ccccc3C43c4ccccc4-c4ccccc43)cc2)cc1]

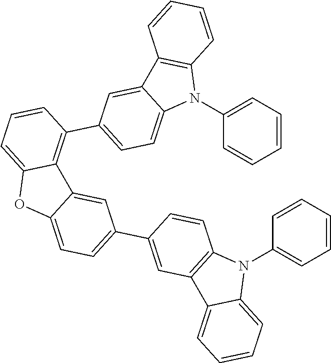 [SMILES: c1ccc(-n2c3ccccc3c3cc(-c4ccc5oc6cccc(-c7ccc8c(c7)c7ccccc7n8-c7ccccc7)c6c5c4)ccc32)cc1]

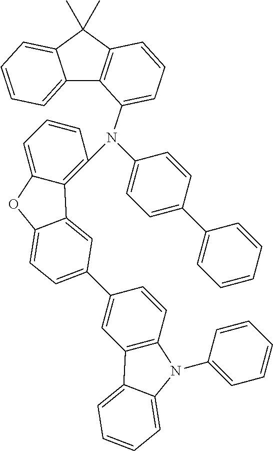 [SMILES: CC1(C)c2ccccc2-c2c(N(c3ccc(-c4ccccc4)cc3)c3cccc4oc5ccc(-c6ccc7c(c6)c6ccccc6n7-c6ccccc6)cc5c34)cccc21]

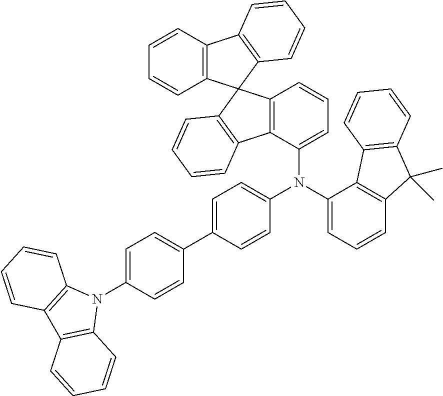 [SMILES: CC1(C)c2ccccc2-c2c(N(c3ccc(-c4ccc(-n5c6ccccc6c6ccccc65)cc4)cc3)c3cccc4c3-c3ccccc3C43c4ccccc4-c4ccccc43)cccc21]

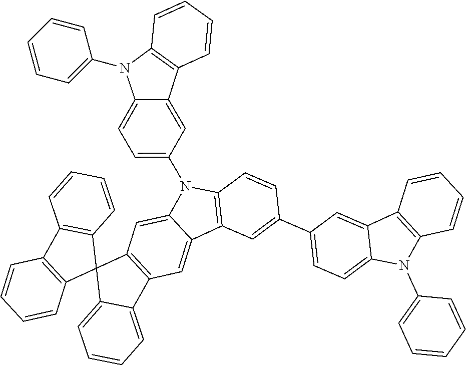 [SMILES: c1ccc(-n2c3ccccc3c3cc(-c4ccc5c(c4)c4cc6c(cc4n5-c4ccc5c(c4)c4ccccc4n5-c4ccccc4)C4(c5ccccc5-c5ccccc54)c4ccccc4-6)ccc32)cc1]

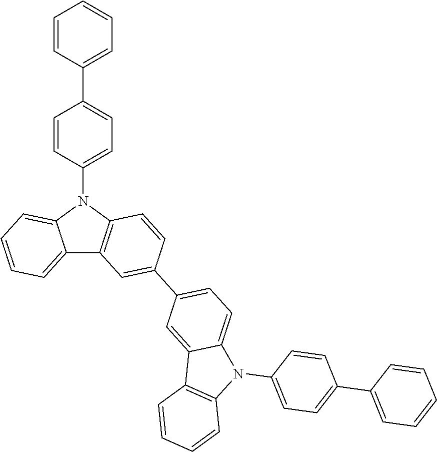 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

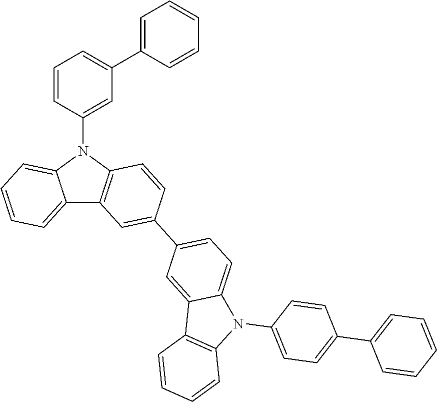 [SMILES: c1ccc(-c2ccc(-n3c4ccccc4c4cc(-c5ccc6c(c5)c5ccccc5n6-c5cccc(-c6ccccc6)c5)ccc43)cc2)cc1]

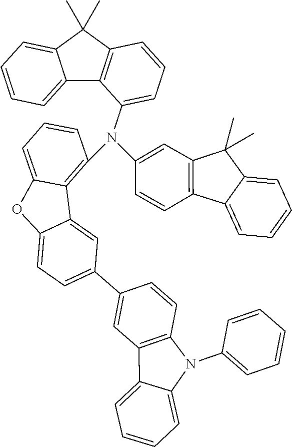 [SMILES: CC1(C)c2ccccc2-c2ccc(N(c3cccc4c3-c3ccccc3C4(C)C)c3cccc4oc5ccc(-c6ccc7c(c6)c6ccccc6n7-c6ccccc6)cc5c34)cc21]

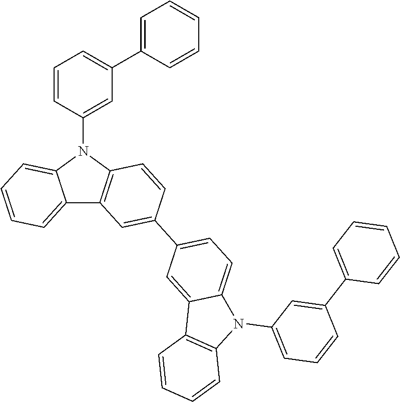 [SMILES: c1ccc(-c2cccc(-n3c4ccccc4c4cc(-c5ccc6c(c5)c5ccccc5n6-c5cccc(-c6ccccc6)c5)ccc43)c2)cc1]

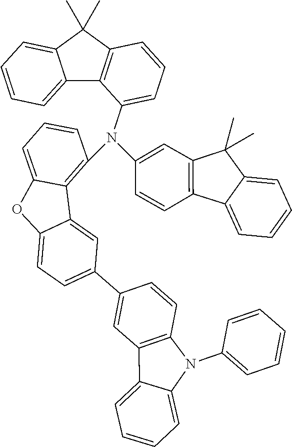 [SMILES: CC1(C)c2ccccc2-c2ccc(N(c3cccc4c3-c3ccccc3C4(C)C)c3cccc4oc5ccc(-c6ccc7c(c6)c6ccccc6n7-c6ccccc6)cc5c34)cc21]

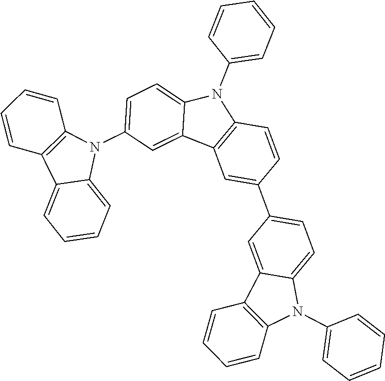 [SMILES: c1ccc(-n2c3ccccc3c3cc(-c4ccc5c(c4)c4cc(-n6c7ccccc7c7ccccc76)ccc4n5-c4ccccc4)ccc32)cc1]

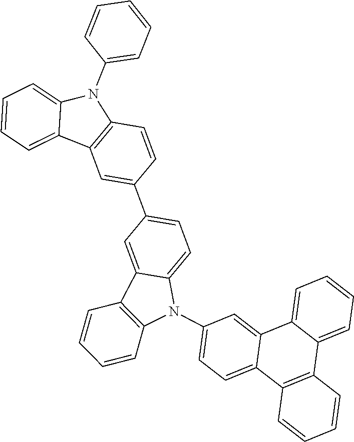 [SMILES: c1ccc(-n2c3ccccc3c3cc(-c4ccc5c(c4)c4ccccc4n5-c4ccc5c6ccccc6c6ccccc6c5c4)ccc32)cc1]

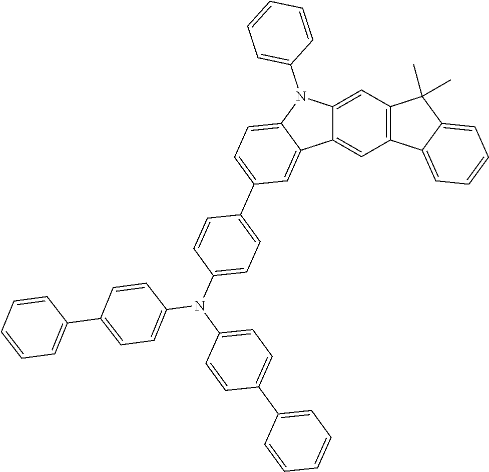 [SMILES: CC1(C)c2ccccc2-c2cc3c4cc(-c5ccc(N(c6ccc(-c7ccccc7)cc6)c6ccc(-c7ccccc7)cc6)cc5)ccc4n(-c4ccccc4)c3cc21]

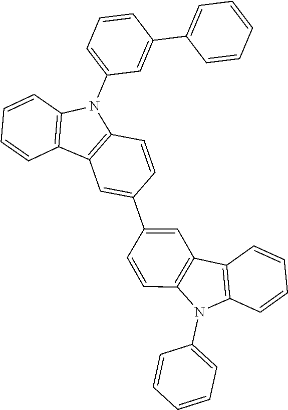 [SMILES: c1ccc(-c2cccc(-n3c4ccccc4c4cc(-c5ccc6c(c5)c5ccccc5n6-c5ccccc5)ccc43)c2)cc1]

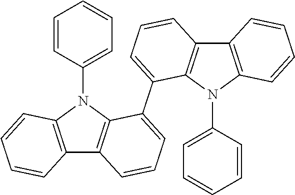 [SMILES: c1ccc(-n2c3ccccc3c3cccc(-c4cccc5c6ccccc6n(-c6ccccc6)c45)c32)cc1]

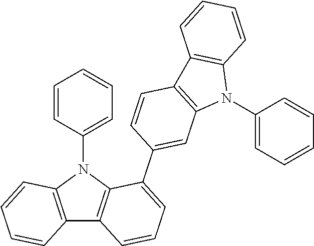 [SMILES: c1ccc(-n2c3ccccc3c3ccc(-c4cccc5c6ccccc6n(-c6ccccc6)c45)cc32)cc1]

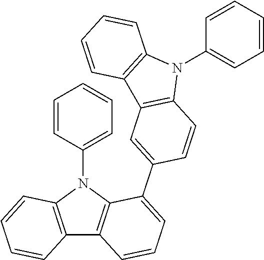 [SMILES: c1ccc(-n2c3ccccc3c3cc(-c4cccc5c6ccccc6n(-c6ccccc6)c45)ccc32)cc1]

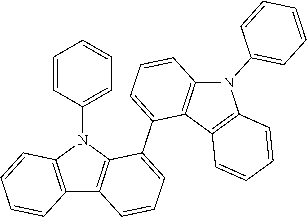 [SMILES: c1ccc(-n2c3ccccc3c3c(-c4cccc5c6ccccc6n(-c6ccccc6)c45)cccc32)cc1]

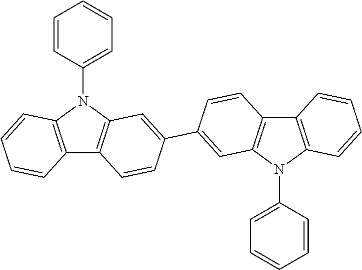 [SMILES: c1ccc(-n2c3ccccc3c3ccc(-c4ccc5c6ccccc6n(-c6ccccc6)c5c4)cc32)cc1]

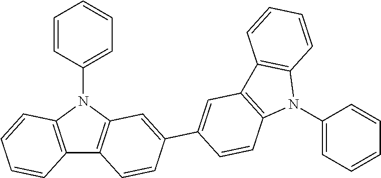 [SMILES: c1ccc(-n2c3ccccc3c3cc(-c4ccc5c6ccccc6n(-c6ccccc6)c5c4)ccc32)cc1]

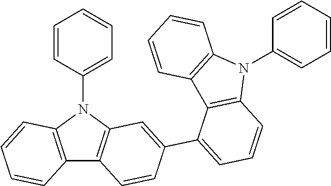 [SMILES: c1ccc(-n2c3ccccc3c3ccc(-c4cccc5c4c4ccccc4n5-c4ccccc4)cc32)cc1]

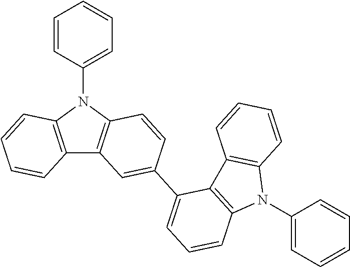 [SMILES: c1ccc(-n2c3ccccc3c3cc(-c4cccc5c4c4ccccc4n5-c4ccccc4)ccc32)cc1]

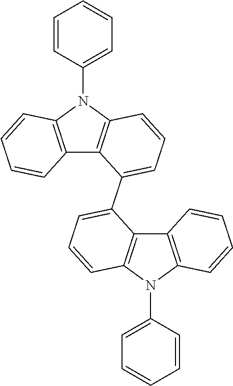 [SMILES: c1ccc(-n2c3ccccc3c3c(-c4cccc5c4c4ccccc4n5-c4ccccc4)cccc32)cc1]

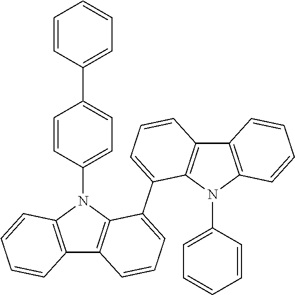 [SMILES: c1ccc(-c2ccc(-n3c4ccccc4c4cccc(-c5cccc6c7ccccc7n(-c7ccccc7)c56)c43)cc2)cc1]

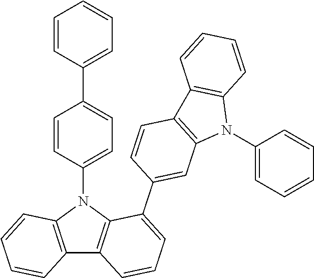 [SMILES: c1ccc(-c2ccc(-n3c4ccccc4c4cccc(-c5ccc6c7ccccc7n(-c7ccccc7)c6c5)c43)cc2)cc1]

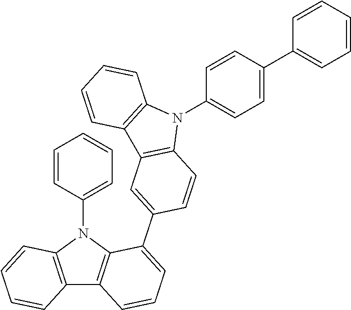 [SMILES: c1ccc(-c2ccc(-n3c4ccccc4c4cc(-c5cccc6c7ccccc7n(-c7ccccc7)c56)ccc43)cc2)cc1]

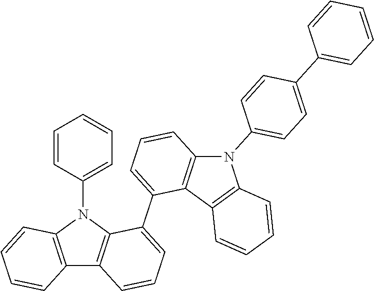 [SMILES: c1ccc(-c2ccc(-n3c4ccccc4c4c(-c5cccc6c7ccccc7n(-c7ccccc7)c56)cccc43)cc2)cc1]

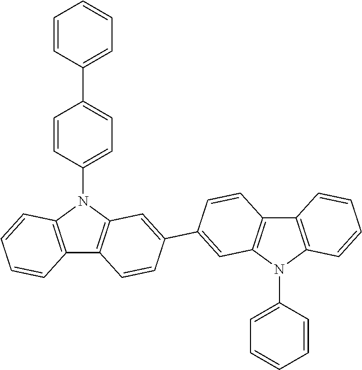 [SMILES: c1ccc(-c2ccc(-n3c4ccccc4c4ccc(-c5ccc6c7ccccc7n(-c7ccccc7)c6c5)cc43)cc2)cc1]

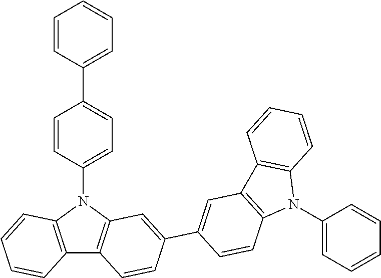 [SMILES: c1ccc(-c2ccc(-n3c4ccccc4c4ccc(-c5ccc6c(c5)c5ccccc5n6-c5ccccc5)cc43)cc2)cc1]

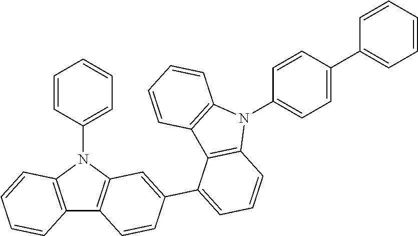 [SMILES: c1ccc(-c2ccc(-n3c4ccccc4c4c(-c5ccc6c7ccccc7n(-c7ccccc7)c6c5)cccc43)cc2)cc1]

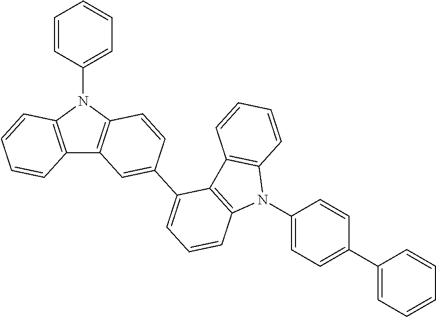 [SMILES: c1ccc(-c2ccc(-n3c4ccccc4c4c(-c5ccc6c(c5)c5ccccc5n6-c5ccccc5)cccc43)cc2)cc1]

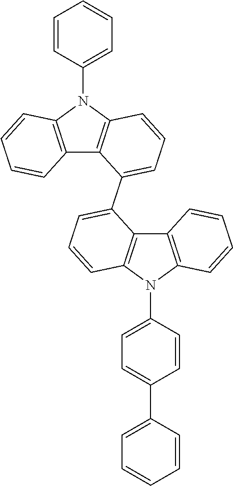 [SMILES: c1ccc(-c2ccc(-n3c4ccccc4c4c(-c5cccc6c5c5ccccc5n6-c5ccccc5)cccc43)cc2)cc1]

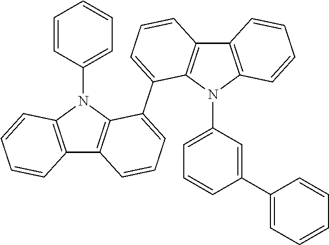 [SMILES: c1ccc(-c2cccc(-n3c4ccccc4c4cccc(-c5cccc6c7ccccc7n(-c7ccccc7)c56)c43)c2)cc1]

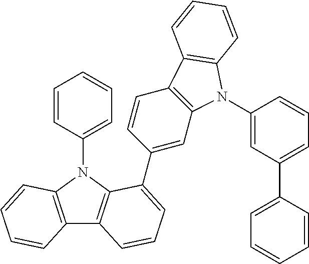 [SMILES: c1ccc(-c2cccc(-n3c4ccccc4c4ccc(-c5cccc6c7ccccc7n(-c7ccccc7)c56)cc43)c2)cc1]

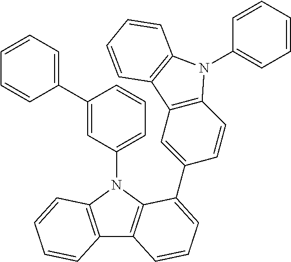 [SMILES: c1ccc(-c2cccc(-n3c4ccccc4c4cccc(-c5ccc6c(c5)c5ccccc5n6-c5ccccc5)c43)c2)cc1]

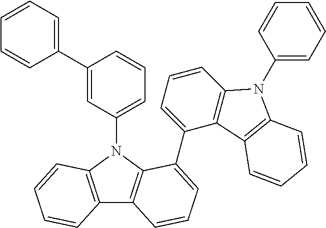 [SMILES: c1ccc(-c2cccc(-n3c4ccccc4c4cccc(-c5cccc6c5c5ccccc5n6-c5ccccc5)c43)c2)cc1]

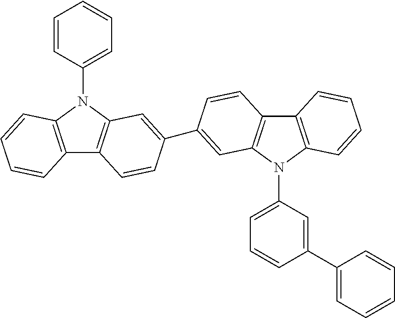 [SMILES: c1ccc(-c2cccc(-n3c4ccccc4c4ccc(-c5ccc6c7ccccc7n(-c7ccccc7)c6c5)cc43)c2)cc1]

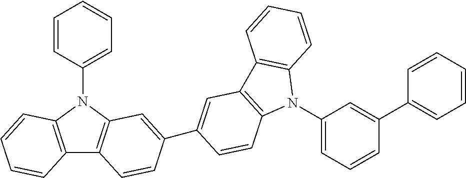 [SMILES: c1ccc(-c2cccc(-n3c4ccccc4c4cc(-c5ccc6c7ccccc7n(-c7ccccc7)c6c5)ccc43)c2)cc1]

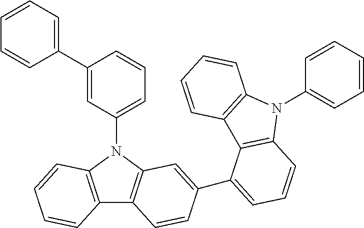 [SMILES: c1ccc(-c2cccc(-n3c4ccccc4c4ccc(-c5cccc6c5c5ccccc5n6-c5ccccc5)cc43)c2)cc1]

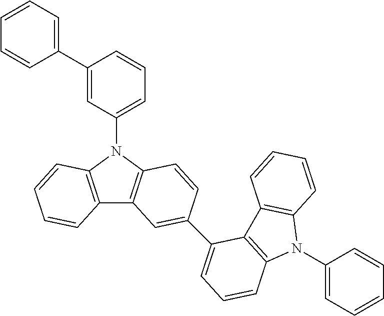 [SMILES: c1ccc(-c2cccc(-n3c4ccccc4c4cc(-c5cccc6c5c5ccccc5n6-c5ccccc5)ccc43)c2)cc1]

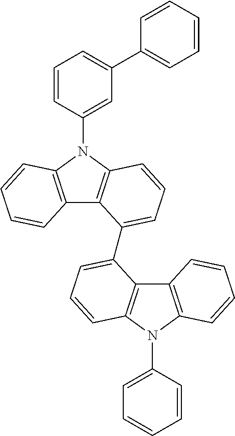 [SMILES: c1ccc(-c2cccc(-n3c4ccccc4c4c(-c5cccc6c5c5ccccc5n6-c5ccccc5)cccc43)c2)cc1]

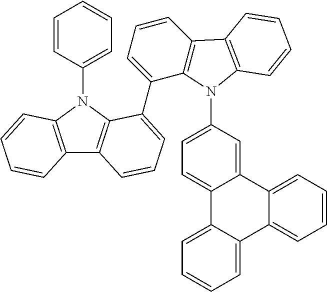 [SMILES: c1ccc(-n2c3ccccc3c3cccc(-c4cccc5c6ccccc6n(-c6ccc7c8ccccc8c8ccccc8c7c6)c45)c32)cc1]

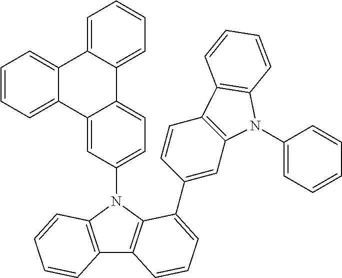 [SMILES: c1ccc(-n2c3ccccc3c3ccc(-c4cccc5c6ccccc6n(-c6ccc7c8ccccc8c8ccccc8c7c6)c45)cc32)cc1]

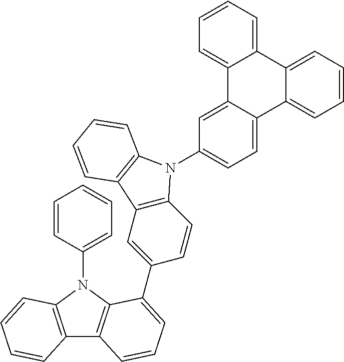 [SMILES: c1ccc(-n2c3ccccc3c3cccc(-c4ccc5c(c4)c4ccccc4n5-c4ccc5c6ccccc6c6ccccc6c5c4)c32)cc1]

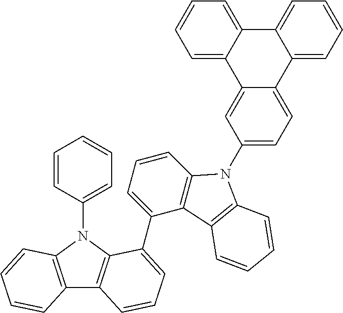 [SMILES: c1ccc(-n2c3ccccc3c3cccc(-c4cccc5c4c4ccccc4n5-c4ccc5c6ccccc6c6ccccc6c5c4)c32)cc1]

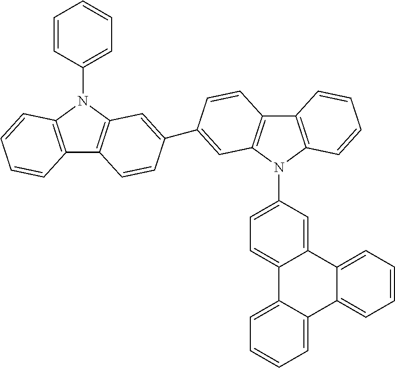 [SMILES: c1ccc(-n2c3ccccc3c3ccc(-c4ccc5c6ccccc6n(-c6ccc7c8ccccc8c8ccccc8c7c6)c5c4)cc32)cc1]

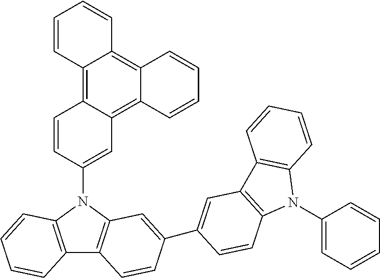 [SMILES: c1ccc(-n2c3ccccc3c3cc(-c4ccc5c6ccccc6n(-c6ccc7c8ccccc8c8ccccc8c7c6)c5c4)ccc32)cc1]

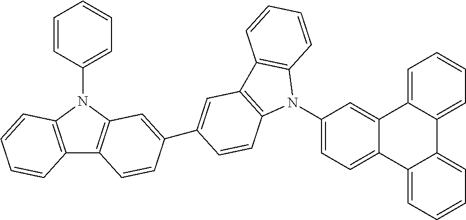 [SMILES: c1ccc(-n2c3ccccc3c3ccc(-c4ccc5c(c4)c4ccccc4n5-c4ccc5c6ccccc6c6ccccc6c5c4)cc32)cc1]

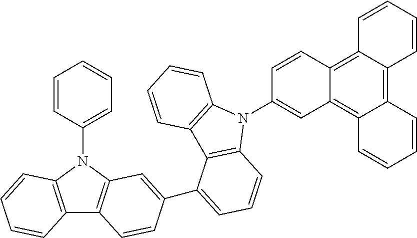 [SMILES: c1ccc(-n2c3ccccc3c3ccc(-c4cccc5c4c4ccccc4n5-c4ccc5c6ccccc6c6ccccc6c5c4)cc32)cc1]

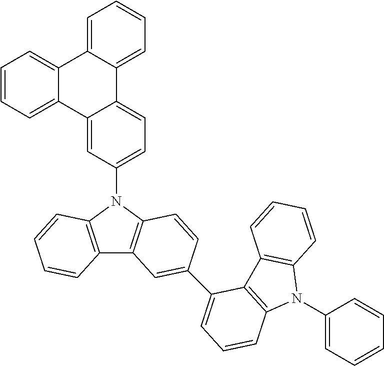 [SMILES: c1ccc(-n2c3ccccc3c3c(-c4ccc5c(c4)c4ccccc4n5-c4ccc5c6ccccc6c6ccccc6c5c4)cccc32)cc1]

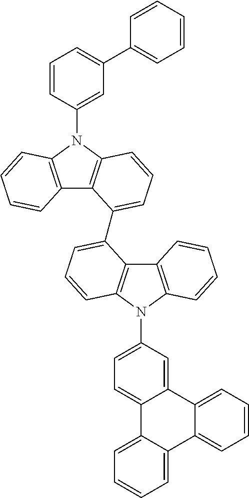 [SMILES: c1ccc(-c2cccc(-n3c4ccccc4c4c(-c5cccc6c5c5ccccc5n6-c5ccc6c7ccccc7c7ccccc7c6c5)cccc43)c2)cc1]

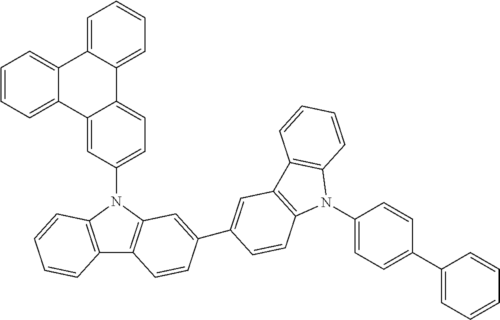 [SMILES: c1ccc(-c2ccc(-n3c4ccccc4c4cc(-c5ccc6c7ccccc7n(-c7ccc8c9ccccc9c9ccccc9c8c7)c6c5)ccc43)cc2)cc1]

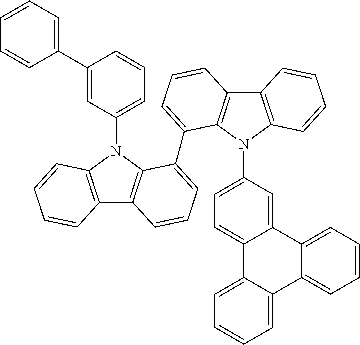 [SMILES: c1ccc(-c2cccc(-n3c4ccccc4c4cccc(-c5cccc6c7ccccc7n(-c7ccc8c9ccccc9c9ccccc9c8c7)c56)c43)c2)cc1]

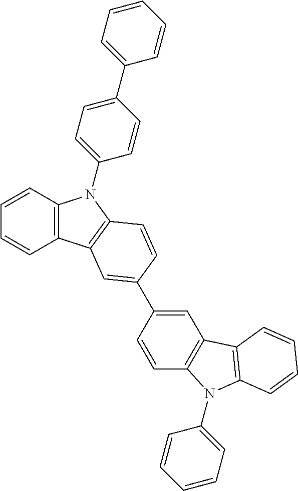 [SMILES: c1ccc(-c2ccc(-n3c4ccccc4c4cc(-c5ccc6c(c5)c5ccccc5n6-c5ccccc5)ccc43)cc2)cc1]

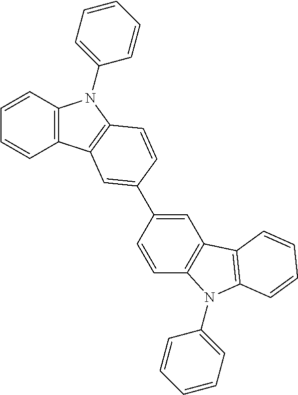 [SMILES: c1ccc(-n2c3ccccc3c3cc(-c4ccc5c(c4)c4ccccc4n5-c4ccccc4)ccc32)cc1]

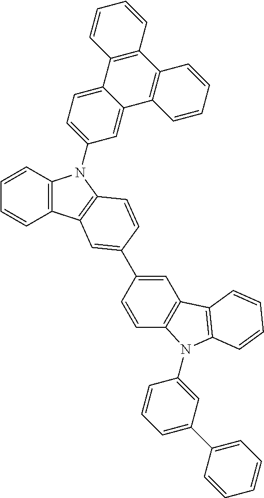 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1]

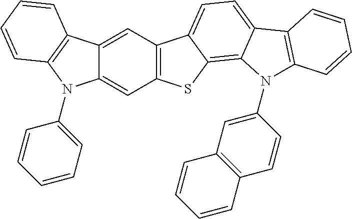 [SMILES: c1ccc(-n2c3ccccc3c3cc4c(cc32)sc2c4ccc3c4ccccc4n(-c4ccc5ccccc5c4)c32)cc1]

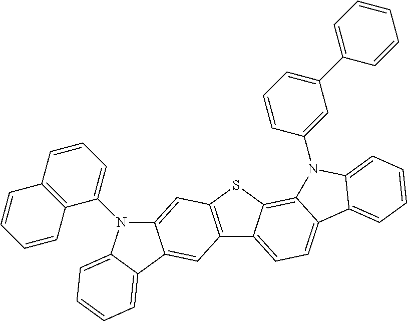 [SMILES: c1ccc(-c2cccc(-n3c4ccccc4c4ccc5c6cc7c8ccccc8n(-c8cccc9ccccc89)c7cc6sc5c43)c2)cc1]

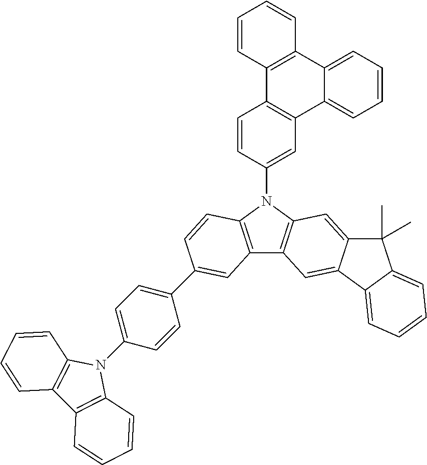 [SMILES: CC1(C)c2ccccc2-c2cc3c4cc(-c5ccc(-n6c7ccccc7c7ccccc76)cc5)ccc4n(-c4ccc5c6ccccc6c6ccccc6c5c4)c3cc21]

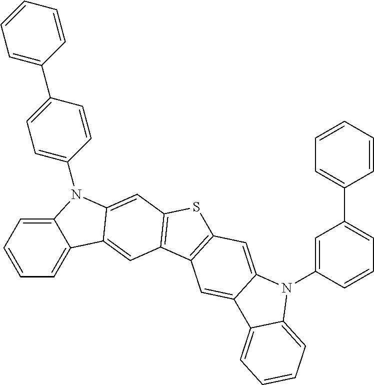 [SMILES: c1ccc(-c2ccc(-n3c4ccccc4c4cc5c(cc43)sc3cc4c(cc35)c3ccccc3n4-c3cccc(-c4ccccc4)c3)cc2)cc1]

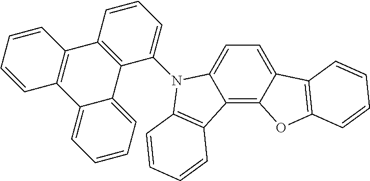 [SMILES: c1ccc2c(c1)oc1c2ccc2c1c1ccccc1n2-c1cccc2c3ccccc3c3ccccc3c12]

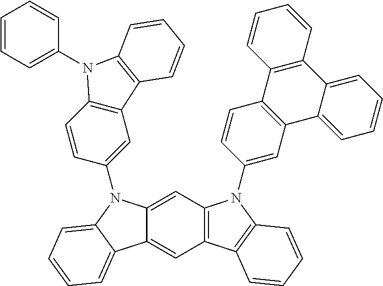 [SMILES: c1ccc(-n2c3ccccc3c3cc(-n4c5ccccc5c5cc6c7ccccc7n(-c7ccc8c9ccccc9c9ccccc9c8c7)c6cc54)ccc32)cc1]

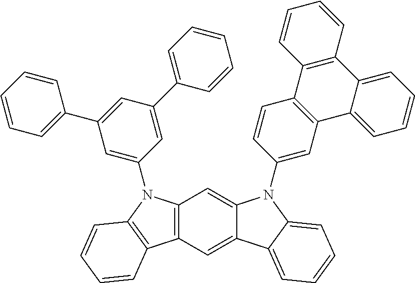 [SMILES: c1ccc(-c2cc(-c3ccccc3)cc(-n3c4ccccc4c4cc5c6ccccc6n(-c6ccc7c8ccccc8c8ccccc8c7c6)c5cc43)c2)cc1]

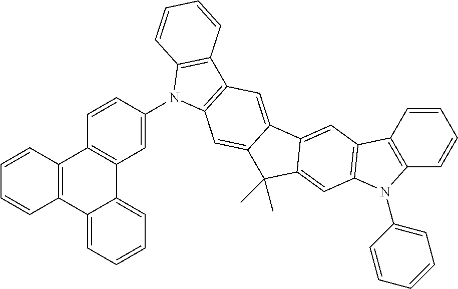 [SMILES: CC1(C)c2cc3c(cc2-c2cc4c5ccccc5n(-c5ccc6c7ccccc7c7ccccc7c6c5)c4cc21)c1ccccc1n3-c1ccccc1]

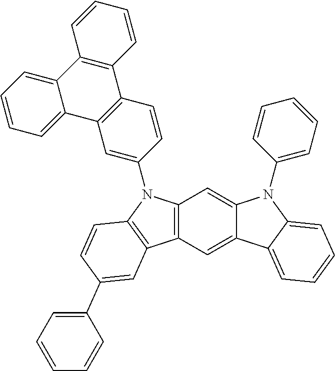 [SMILES: c1ccc(-c2ccc3c(c2)c2cc4c5ccccc5n(-c5ccccc5)c4cc2n3-c2ccc3c4ccccc4c4ccccc4c3c2)cc1]

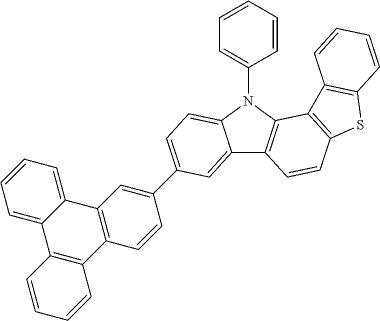 [SMILES: c1ccc(-n2c3ccc(-c4ccc5c6ccccc6c6ccccc6c5c4)cc3c3ccc4sc5ccccc5c4c32)cc1]

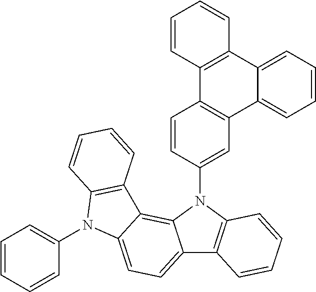 [SMILES: c1ccc(-n2c3ccccc3c3c2ccc2c4ccccc4n(-c4ccc5c6ccccc6c6ccccc6c5c4)c23)cc1]

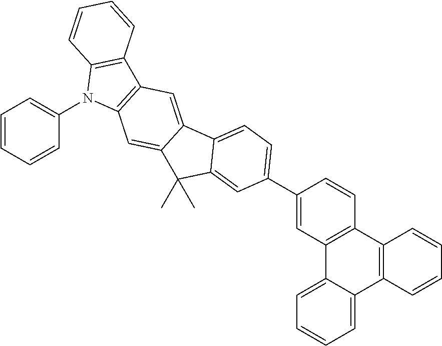 [SMILES: CC1(C)c2cc(-c3ccc4c5ccccc5c5ccccc5c4c3)ccc2-c2cc3c4ccccc4n(-c4ccccc4)c3cc21]

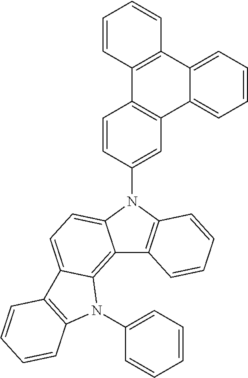 [SMILES: c1ccc(-n2c3ccccc3c3ccc4c(c5ccccc5n4-c4ccc5c6ccccc6c6ccccc6c5c4)c32)cc1]

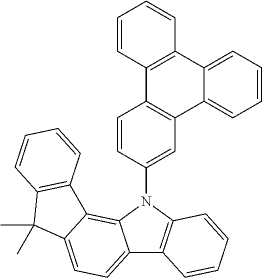 [SMILES: CC1(C)c2ccccc2-c2c1ccc1c3ccccc3n(-c3ccc4c5ccccc5c5ccccc5c4c3)c21]

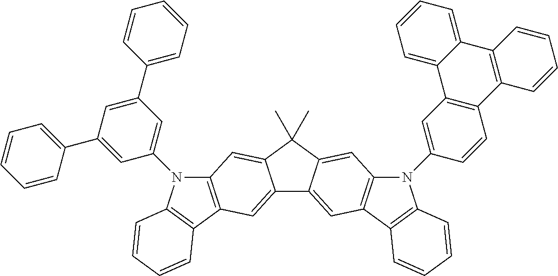 [SMILES: CC1(C)c2cc3c(cc2-c2cc4c5ccccc5n(-c5ccc6c7ccccc7c7ccccc7c6c5)c4cc21)c1ccccc1n3-c1cc(-c2ccccc2)cc(-c2ccccc2)c1]

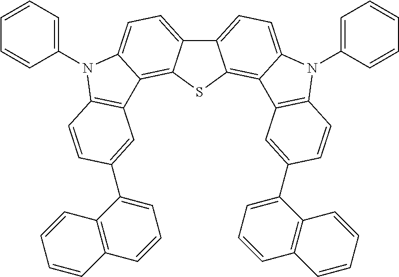 [SMILES: c1ccc(-n2c3ccc(-c4cccc5ccccc45)cc3c3c4sc5c(ccc6c5c5cc(-c7cccc8ccccc78)ccc5n6-c5ccccc5)c4ccc32)cc1]

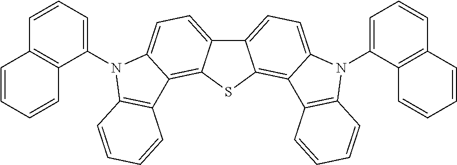 [SMILES: c1ccc2c(-n3c4ccccc4c4c5sc6c(ccc7c6c6ccccc6n7-c6cccc7ccccc67)c5ccc43)cccc2c1]

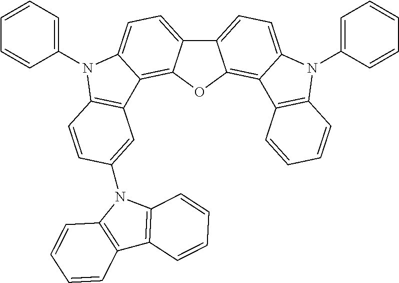 [SMILES: c1ccc(-n2c3ccccc3c3c4oc5c(ccc6c5c5cc(-n7c8ccccc8c8ccccc87)ccc5n6-c5ccccc5)c4ccc32)cc1]

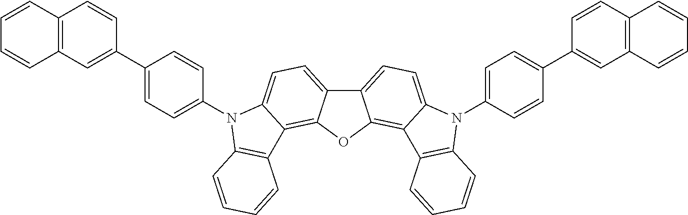 [SMILES: c1ccc2cc(-c3ccc(-n4c5ccccc5c5c6oc7c(ccc8c7c7ccccc7n8-c7ccc(-c8ccc9ccccc9c8)cc7)c6ccc54)cc3)ccc2c1]

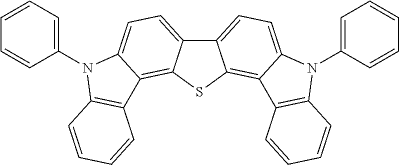 [SMILES: c1ccc(-n2c3ccccc3c3c4sc5c(ccc6c5c5ccccc5n6-c5ccccc5)c4ccc32)cc1]

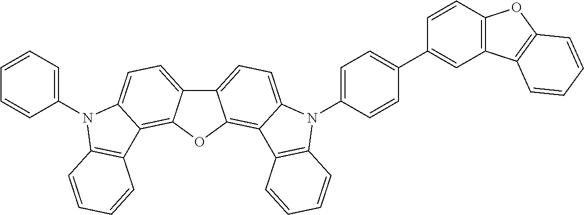 [SMILES: c1ccc(-n2c3ccccc3c3c4oc5c(ccc6c5c5ccccc5n6-c5ccc(-c6ccc7oc8ccccc8c7c6)cc5)c4ccc32)cc1]

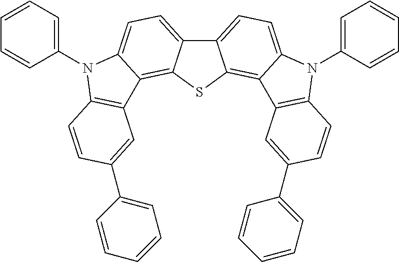 [SMILES: c1ccc(-c2ccc3c(c2)c2c4sc5c(ccc6c5c5cc(-c7ccccc7)ccc5n6-c5ccccc5)c4ccc2n3-c2ccccc2)cc1]

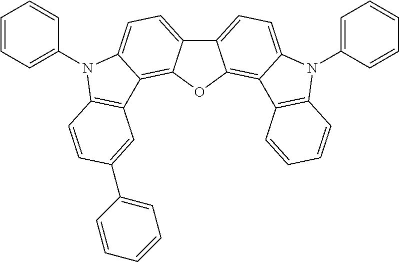 [SMILES: c1ccc(-c2ccc3c(c2)c2c4oc5c(ccc6c5c5ccccc5n6-c5ccccc5)c4ccc2n3-c2ccccc2)cc1]

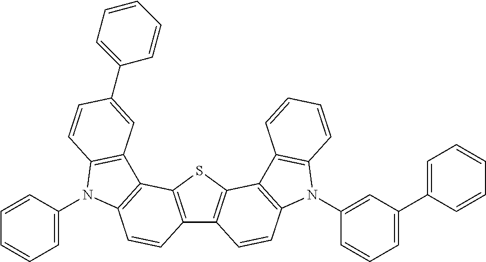 [SMILES: c1ccc(-c2cccc(-n3c4ccccc4c4c5sc6c(ccc7c6c6cc(-c8ccccc8)ccc6n7-c6ccccc6)c5ccc43)c2)cc1]

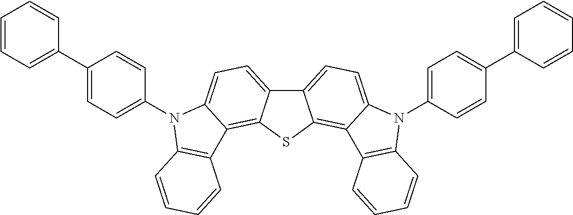 [SMILES: c1ccc(-c2ccc(-n3c4ccccc4c4c5sc6c(ccc7c6c6ccccc6n7-c6ccc(-c7ccccc7)cc6)c5ccc43)cc2)cc1]

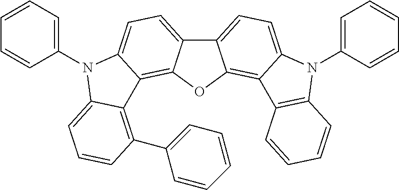 [SMILES: c1ccc(-c2cccc3c2c2c4oc5c(ccc6c5c5ccccc5n6-c5ccccc5)c4ccc2n3-c2ccccc2)cc1]

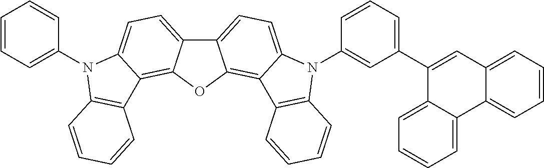 [SMILES: c1ccc(-n2c3ccccc3c3c4oc5c(ccc6c5c5ccccc5n6-c5cccc(-c6cc7ccccc7c7ccccc67)c5)c4ccc32)cc1]

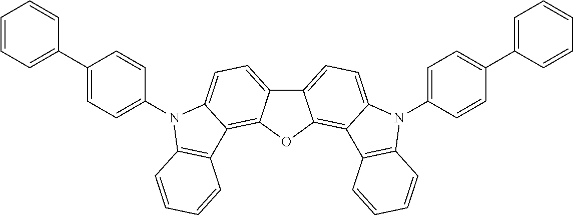 [SMILES: c1ccc(-c2ccc(-n3c4ccccc4c4c5oc6c(ccc7c6c6ccccc6n7-c6ccc(-c7ccccc7)cc6)c5ccc43)cc2)cc1]

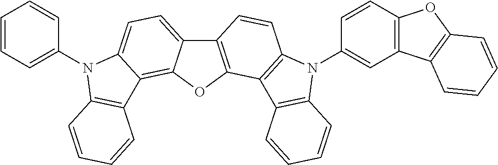 [SMILES: c1ccc(-n2c3ccccc3c3c4oc5c(ccc6c5c5ccccc5n6-c5ccc6oc7ccccc7c6c5)c4ccc32)cc1]

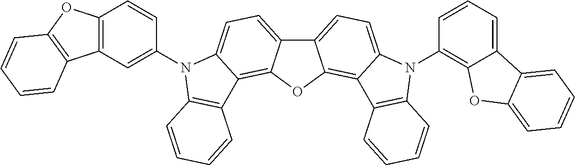 [SMILES: c1ccc2c(c1)oc1ccc(-n3c4ccccc4c4c5oc6c(ccc7c6c6ccccc6n7-c6cccc7c6oc6ccccc67)c5ccc43)cc12]

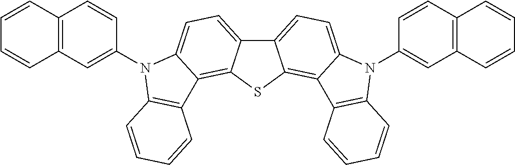 [SMILES: c1ccc2cc(-n3c4ccccc4c4c5sc6c(ccc7c6c6ccccc6n7-c6ccc7ccccc7c6)c5ccc43)ccc2c1]

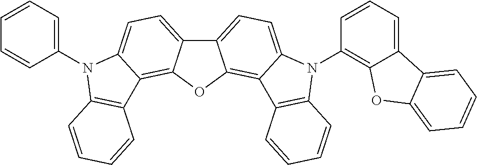 [SMILES: c1ccc(-n2c3ccccc3c3c4oc5c(ccc6c5c5ccccc5n6-c5cccc6c5oc5ccccc56)c4ccc32)cc1]

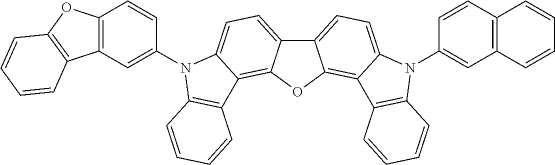 [SMILES: c1ccc2cc(-n3c4ccccc4c4c5oc6c(ccc7c6c6ccccc6n7-c6ccc7oc8ccccc8c7c6)c5ccc43)ccc2c1]

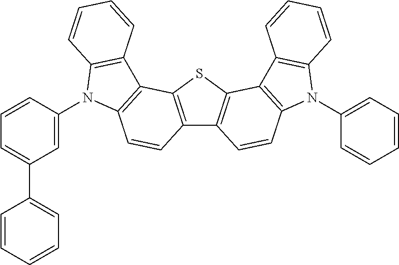 [SMILES: c1ccc(-c2cccc(-n3c4ccccc4c4c5sc6c(ccc7c6c6ccccc6n7-c6ccccc6)c5ccc43)c2)cc1]

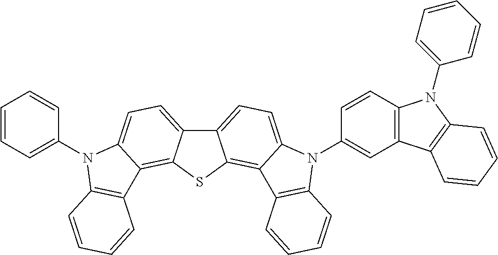 [SMILES: c1ccc(-n2c3ccccc3c3cc(-n4c5ccccc5c5c6sc7c(ccc8c7c7ccccc7n8-c7ccccc7)c6ccc54)ccc32)cc1]

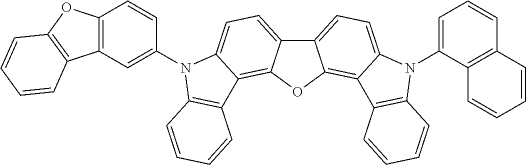 [SMILES: c1ccc2c(-n3c4ccccc4c4c5oc6c(ccc7c6c6ccccc6n7-c6ccc7oc8ccccc8c7c6)c5ccc43)cccc2c1]

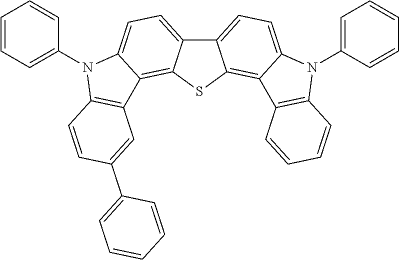 [SMILES: c1ccc(-c2ccc3c(c2)c2c4sc5c(ccc6c5c5ccccc5n6-c5ccccc5)c4ccc2n3-c2ccccc2)cc1]

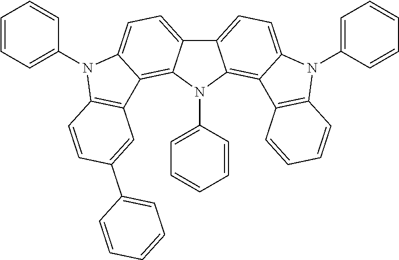 [SMILES: c1ccc(-c2ccc3c(c2)c2c(ccc4c5ccc6c(c7ccccc7n6-c6ccccc6)c5n(-c5ccccc5)c42)n3-c2ccccc2)cc1]

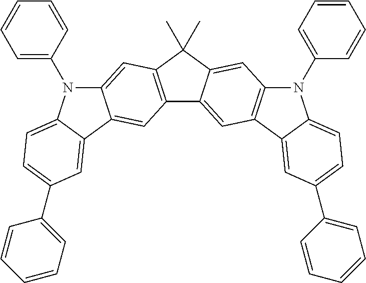 [SMILES: CC1(C)c2cc3c(cc2-c2cc4c5cc(-c6ccccc6)ccc5n(-c5ccccc5)c4cc21)c1cc(-c2ccccc2)ccc1n3-c1ccccc1]

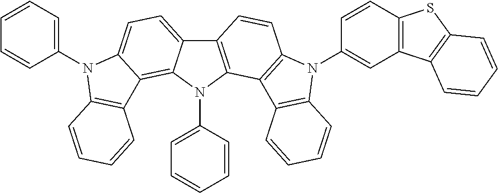 [SMILES: c1ccc(-n2c3ccccc3c3c2ccc2c4ccc5c(c6ccccc6n5-c5ccc6sc7ccccc7c6c5)c4n(-c4ccccc4)c23)cc1]

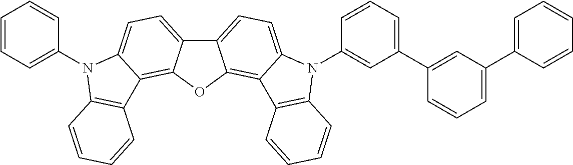 [SMILES: c1ccc(-c2cccc(-c3cccc(-n4c5ccccc5c5c6oc7c(ccc8c7c7ccccc7n8-c7ccccc7)c6ccc54)c3)c2)cc1]

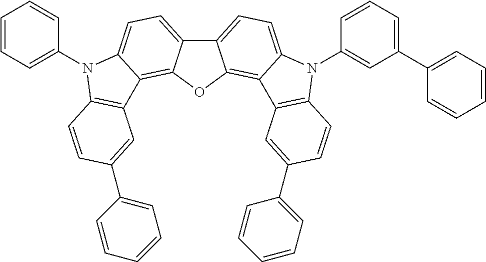 [SMILES: c1ccc(-c2cccc(-n3c4ccc(-c5ccccc5)cc4c4c5oc6c(ccc7c6c6cc(-c8ccccc8)ccc6n7-c6ccccc6)c5ccc43)c2)cc1]

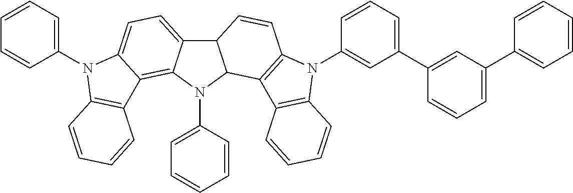 [SMILES: C1=CC2c3ccc4c(c3N(c3ccccc3)C2c2c1n(-c1cccc(-c3cccc(-c5ccccc5)c3)c1)c1ccccc21)c1ccccc1n4-c1ccccc1]

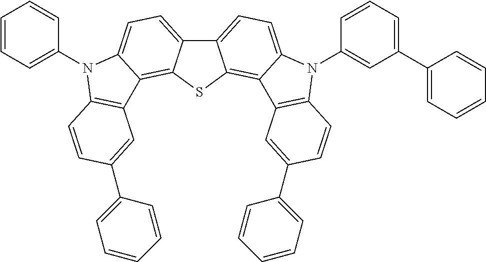 [SMILES: c1ccc(-c2cccc(-n3c4ccc(-c5ccccc5)cc4c4c5sc6c(ccc7c6c6cc(-c8ccccc8)ccc6n7-c6ccccc6)c5ccc43)c2)cc1]

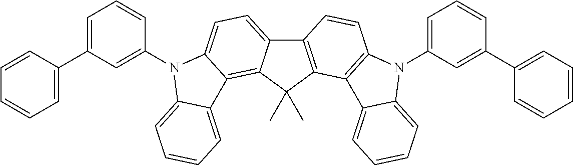 [SMILES: CC1(C)c2c(ccc3c2c2ccccc2n3-c2cccc(-c3ccccc3)c2)-c2ccc3c(c21)c1ccccc1n3-c1cccc(-c2ccccc2)c1]

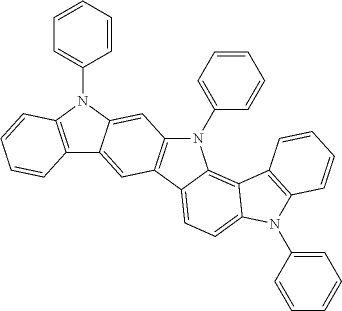 [SMILES: c1ccc(-n2c3ccccc3c3cc4c5ccc6c(c7ccccc7n6-c6ccccc6)c5n(-c5ccccc5)c4cc32)cc1]

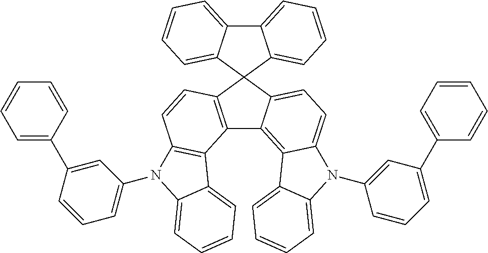 [SMILES: c1ccc(-c2cccc(-n3c4ccccc4c4c5c(ccc43)C3(c4ccccc4-c4ccccc43)c3ccc4c(c3-5)c3ccccc3n4-c3cccc(-c4ccccc4)c3)c2)cc1]

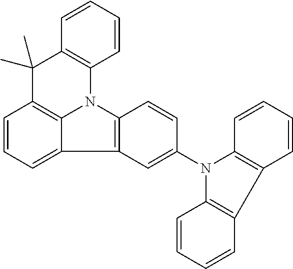 [SMILES: CC1(C)c2ccccc2-n2c3ccc(-n4c5ccccc5c5ccccc54)cc3c3cccc1c32]

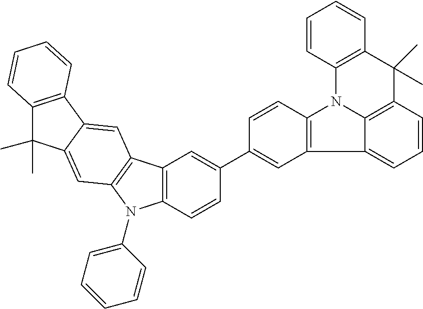

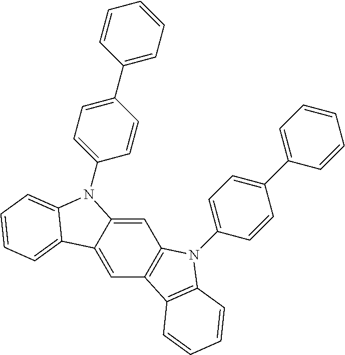 [SMILES: c1ccc(-c2ccc(-n3c4ccccc4c4cc5c6ccccc6n(-c6ccc(-c7ccccc7)cc6)c5cc43)cc2)cc1]

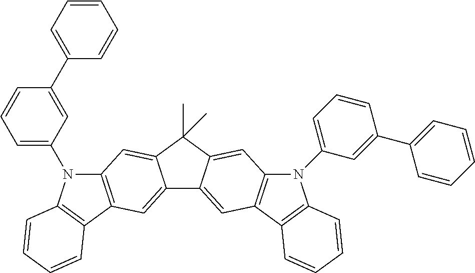 [SMILES: CC1(C)c2cc3c(cc2-c2cc4c5ccccc5n(-c5cccc(-c6ccccc6)c5)c4cc21)c1ccccc1n3-c1cccc(-c2ccccc2)c1]

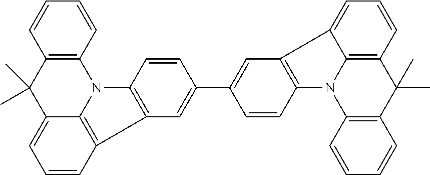

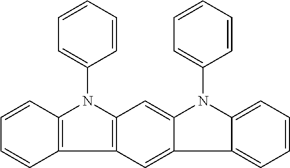 [SMILES: c1ccc(-n2c3ccccc3c3cc4c5ccccc5n(-c5ccccc5)c4cc32)cc1]

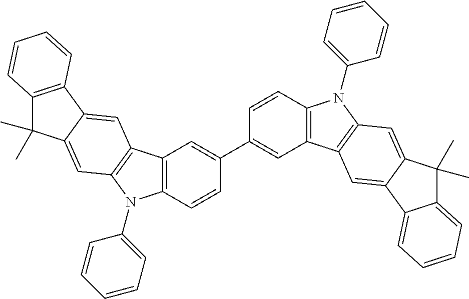

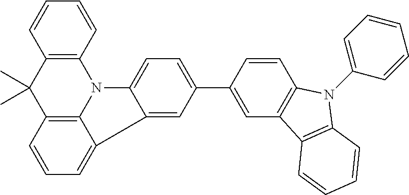 [SMILES: CC1(C)c2ccccc2-n2c3ccc(-c4ccc5c(c4)c4ccccc4n5-c4ccccc4)cc3c3cccc1c32]

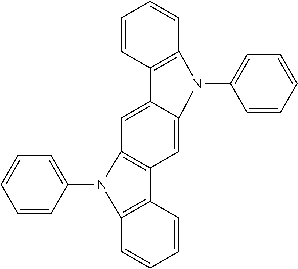 [SMILES: c1ccc(-n2c3ccccc3c3cc4c(cc32)c2ccccc2n4-c2ccccc2)cc1]

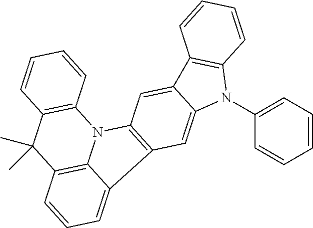 [SMILES: CC1(C)c2ccccc2-n2c3cc4c5ccccc5n(-c5ccccc5)c4cc3c3cccc1c32]

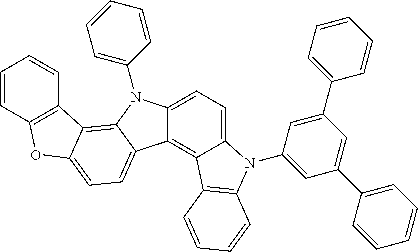 [SMILES: c1ccc(-c2cc(-c3ccccc3)cc(-n3c4ccccc4c4c5c6ccc7oc8ccccc8c7c6n(-c6ccccc6)c5ccc43)c2)cc1]

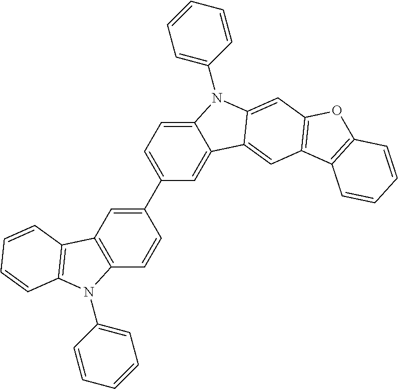 [SMILES: c1ccc(-n2c3ccccc3c3cc(-c4ccc5c(c4)c4cc6c(cc4n5-c4ccccc4)oc4ccccc46)ccc32)cc1]

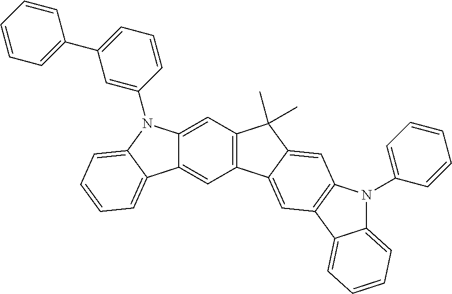 [SMILES: CC1(C)c2cc3c(cc2-c2cc4c5ccccc5n(-c5cccc(-c6ccccc6)c5)c4cc21)c1ccccc1n3-c1ccccc1]

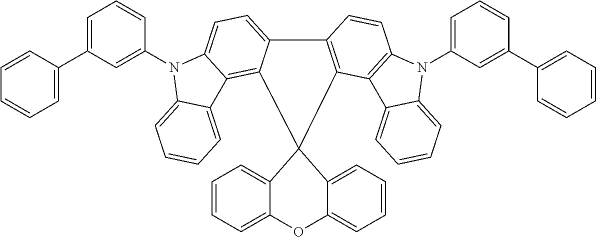 [SMILES: c1ccc(-c2cccc(-n3c4ccccc4c4c5c(ccc43)-c3ccc4c(c3C53c5ccccc5Oc5ccccc53)c3ccccc3n4-c3cccc(-c4ccccc4)c3)c2)cc1]

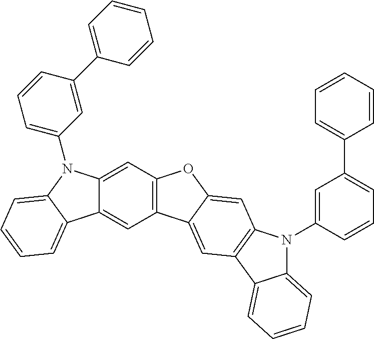 [SMILES: c1ccc(-c2cccc(-n3c4ccccc4c4cc5c(cc43)oc3cc4c(cc35)c3ccccc3n4-c3cccc(-c4ccccc4)c3)c2)cc1]

The table that follows shows examples of suitable wide-bandgap matrix materials that can be used together with the materials of the invention.

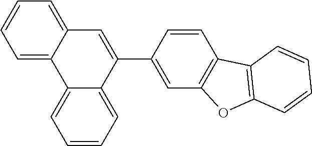 [SMILES: c1ccc2c(c1)cc(-c1ccc3c(c1)oc1ccccc13)c1ccccc12]

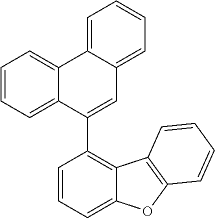 [SMILES: c1ccc2c(c1)cc(-c1cccc3oc4ccccc4c13)c1ccccc12]

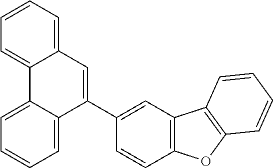 [SMILES: c1ccc2c(c1)cc(-c1ccc3oc4ccccc4c3c1)c1ccccc12]

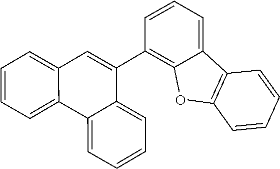 [SMILES: c1ccc2c(c1)cc(-c1cccc3c1oc1ccccc13)c1ccccc12]

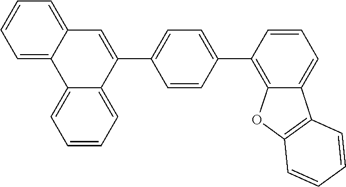 [SMILES: c1ccc2c(c1)cc(-c1ccc(-c3cccc4c3oc3ccccc34)cc1)c1ccccc12]

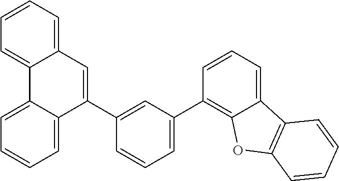 [SMILES: c1cc(-c2cc3ccccc3c3ccccc23)cc(-c2cccc3c2oc2ccccc23)c1]

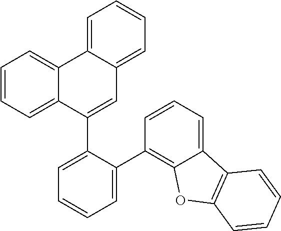 [SMILES: c1ccc(-c2cccc3c2oc2ccccc23)c(-c2cc3ccccc3c3ccccc23)c1]

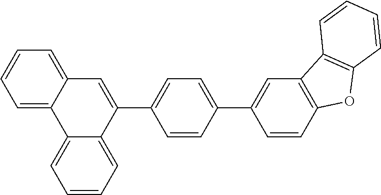 [SMILES: c1ccc2c(c1)cc(-c1ccc(-c3ccc4oc5ccccc5c4c3)cc1)c1ccccc12]

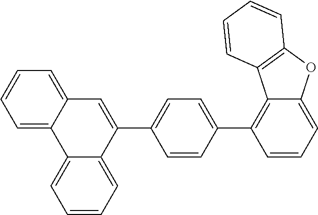 [SMILES: c1ccc2c(c1)cc(-c1ccc(-c3cccc4oc5ccccc5c34)cc1)c1ccccc12]

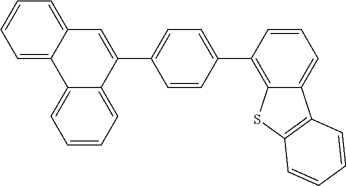 [SMILES: c1ccc2c(c1)cc(-c1ccc(-c3cccc4c3sc3ccccc34)cc1)c1ccccc12]

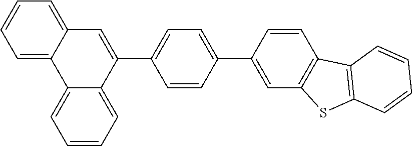 [SMILES: c1ccc2c(c1)cc(-c1ccc(-c3ccc4c(c3)sc3ccccc34)cc1)c1ccccc12]

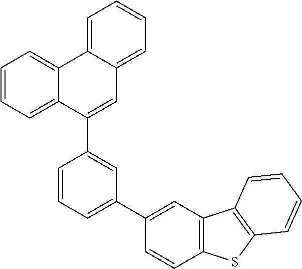 [SMILES: c1cc(-c2ccc3sc4ccccc4c3c2)cc(-c2cc3ccccc3c3ccccc23)c1]

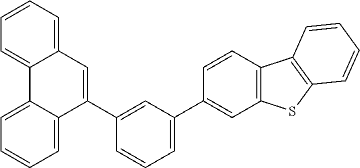 [SMILES: c1cc(-c2ccc3c(c2)sc2ccccc23)cc(-c2cc3ccccc3c3ccccc23)c1]

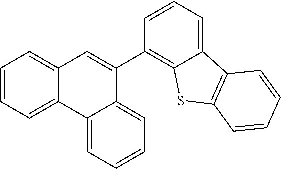 [SMILES: c1ccc2c(c1)cc(-c1cccc3c1sc1ccccc13)c1ccccc12]

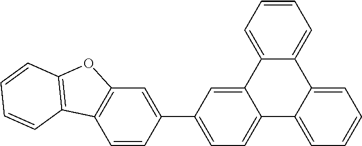 [SMILES: c1ccc2c(c1)oc1cc(-c3ccc4c5ccccc5c5ccccc5c4c3)ccc12]

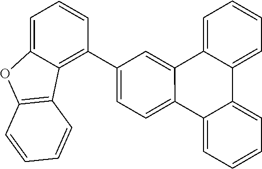 [SMILES: c1ccc2c(c1)oc1cccc(-c3ccc4c5ccccc5c5ccccc5c4c3)c12]

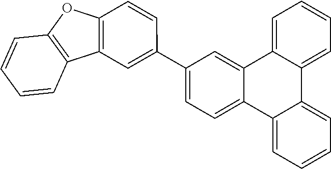 [SMILES: c1ccc2c(c1)oc1ccc(-c3ccc4c5ccccc5c5ccccc5c4c3)cc12]

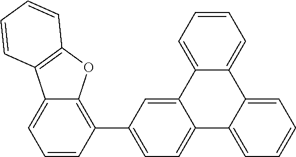 [SMILES: c1ccc2c(c1)oc1c(-c3ccc4c5ccccc5c5ccccc5c4c3)cccc12]

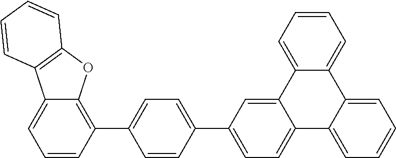 [SMILES: c1ccc2c(c1)oc1c(-c3ccc(-c4ccc5c6ccccc6c6ccccc6c5c4)cc3)cccc12]

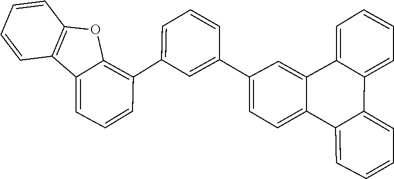 [SMILES: c1cc(-c2ccc3c4ccccc4c4ccccc4c3c2)cc(-c2cccc3c2oc2ccccc23)c1]

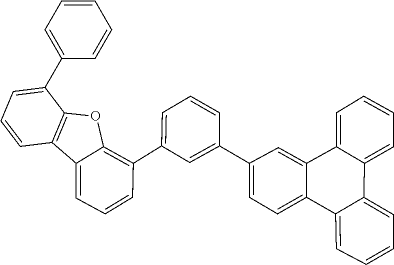 [SMILES: c1ccc(-c2cccc3c2oc2c(-c4cccc(-c5ccc6c7ccccc7c7ccccc7c6c5)c4)cccc23)cc1]

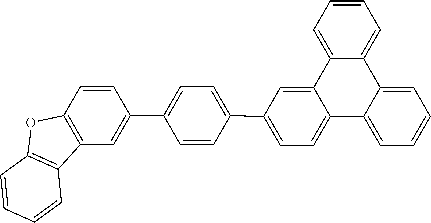 [SMILES: c1ccc2c(c1)oc1ccc(-c3ccc(-c4ccc5c6ccccc6c6ccccc6c5c4)cc3)cc12]

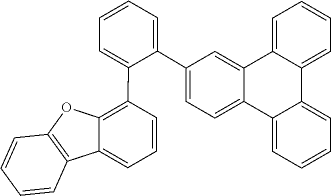 [SMILES: c1ccc(-c2cccc3c2oc2ccccc23)c(-c2ccc3c4ccccc4c4ccccc4c3c2)c1]

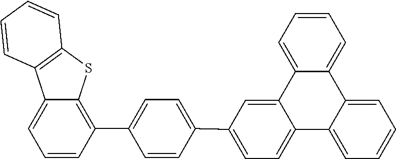 [SMILES: c1ccc2c(c1)sc1c(-c3ccc(-c4ccc5c6ccccc6c6ccccc6c5c4)cc3)cccc12]

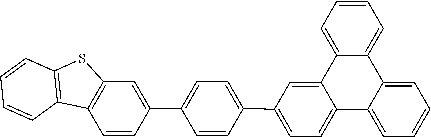 [SMILES: c1ccc2c(c1)sc1cc(-c3ccc(-c4ccc5c6ccccc6c6ccccc6c5c4)cc3)ccc12]

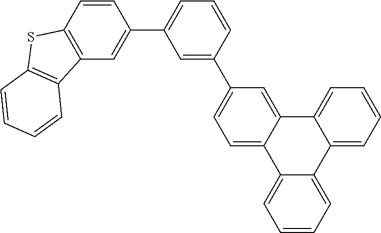 [SMILES: c1cc(-c2ccc3sc4ccccc4c3c2)cc(-c2ccc3c4ccccc4c4ccccc4c3c2)c1]

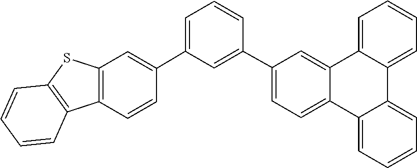 [SMILES: c1cc(-c2ccc3c(c2)sc2ccccc23)cc(-c2ccc3c4ccccc4c4ccccc4c3c2)c1]

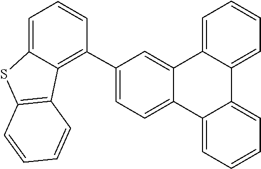 [SMILES: c1ccc2c(c1)sc1cccc(-c3ccc4c5ccccc5c5ccccc5c4c3)c12]

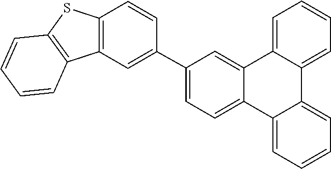 [SMILES: c1ccc2c(c1)sc1ccc(-c3ccc4c5ccccc5c5ccccc5c4c3)cc12]

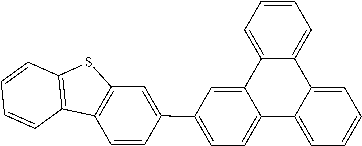 [SMILES: c1ccc2c(c1)sc1cc(-c3ccc4c5ccccc5c5ccccc5c4c3)ccc12]

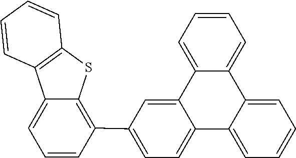 [SMILES: c1ccc2c(c1)sc1c(-c3ccc4c5ccccc5c5ccccc5c4c3)cccc12]

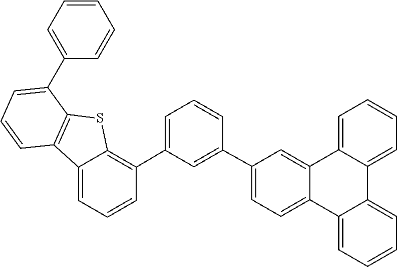 [SMILES: c1ccc(-c2cccc3c2sc2c(-c4cccc(-c5ccc6c7ccccc7c7ccccc7c6c5)c4)cccc23)cc1]

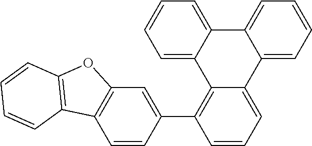 [SMILES: c1ccc2c(c1)oc1cc(-c3cccc4c5ccccc5c5ccccc5c34)ccc12]

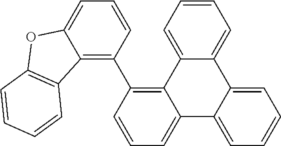 [SMILES: c1ccc2c(c1)oc1cccc(-c3cccc4c5ccccc5c5ccccc5c34)c12]

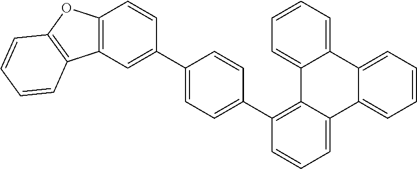 [SMILES: c1ccc2c(c1)oc1ccc(-c3ccc(-c4cccc5c6ccccc6c6ccccc6c45)cc3)cc12]

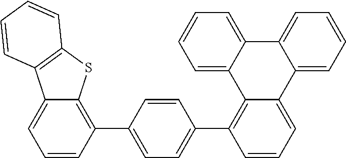 [SMILES: c1ccc2c(c1)sc1c(-c3ccc(-c4cccc5c6ccccc6c6ccccc6c45)cc3)cccc12]

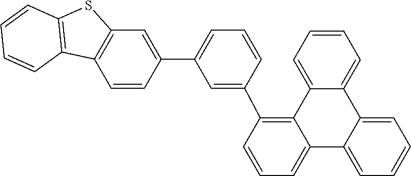 [SMILES: c1cc(-c2ccc3c(c2)sc2ccccc23)cc(-c2cccc3c4ccccc4c4ccccc4c23)c1]

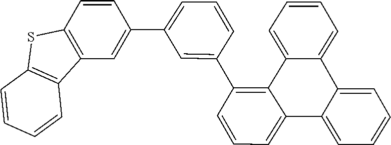 [SMILES: c1cc(-c2ccc3sc4ccccc4c3c2)cc(-c2cccc3c4ccccc4c4ccccc4c23)c1]

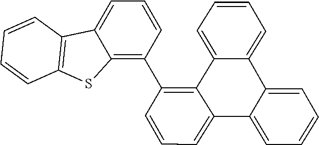 [SMILES: c1ccc2c(c1)sc1c(-c3cccc4c5ccccc5c5ccccc5c34)cccc12]

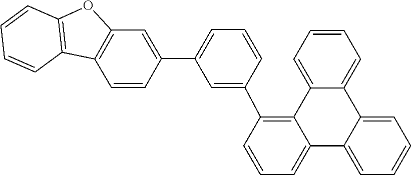 [SMILES: c1cc(-c2ccc3c(c2)oc2ccccc23)cc(-c2cccc3c4ccccc4c4ccccc4c23)c1]

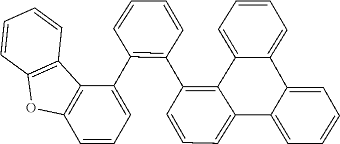 [SMILES: c1ccc(-c2cccc3c4ccccc4c4ccccc4c23)c(-c2cccc3oc4ccccc4c23)c1]

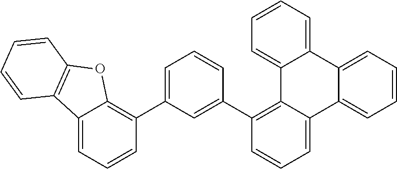 [SMILES: c1cc(-c2cccc3c2oc2ccccc23)cc(-c2cccc3c4ccccc4c4ccccc4c23)c1]

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186 and WO 2018/041769, and as yet unpublished patent applications EP 17182995.5, EP 17205103.9, EP 17206950.2 and EP 18156388.3. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

Examples of phosphorescent dopants are adduced below.

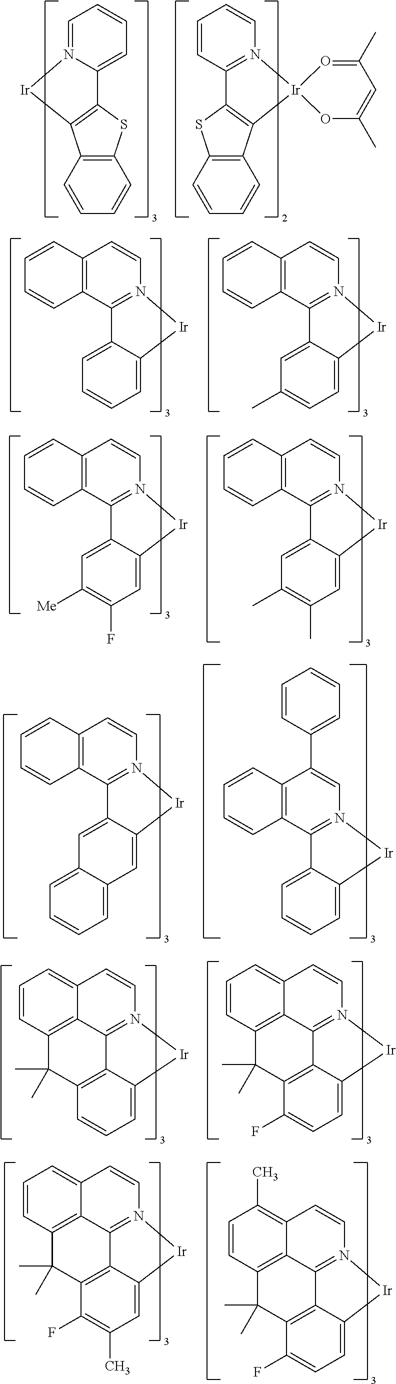

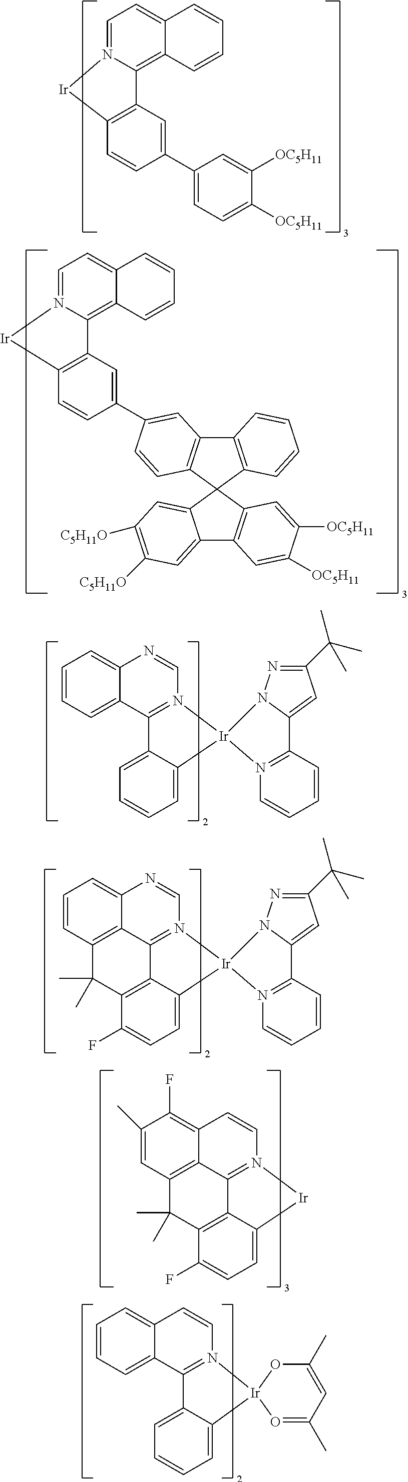

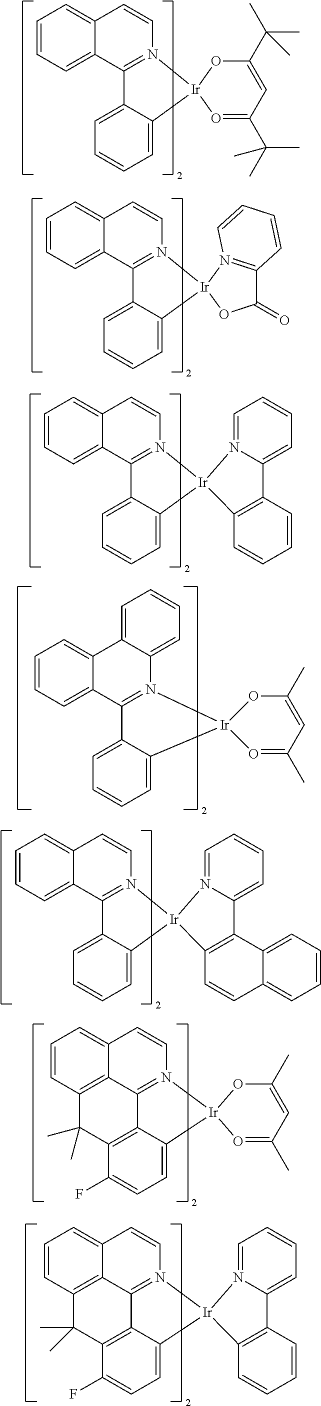

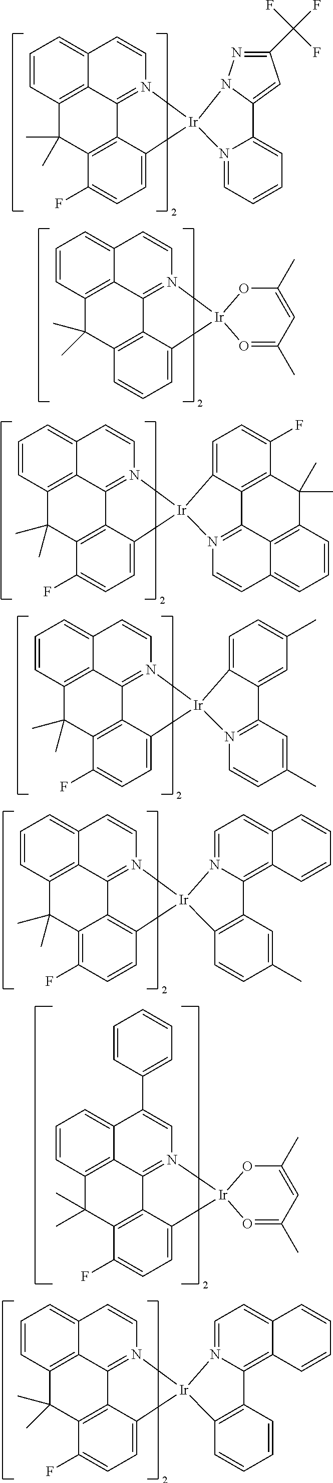

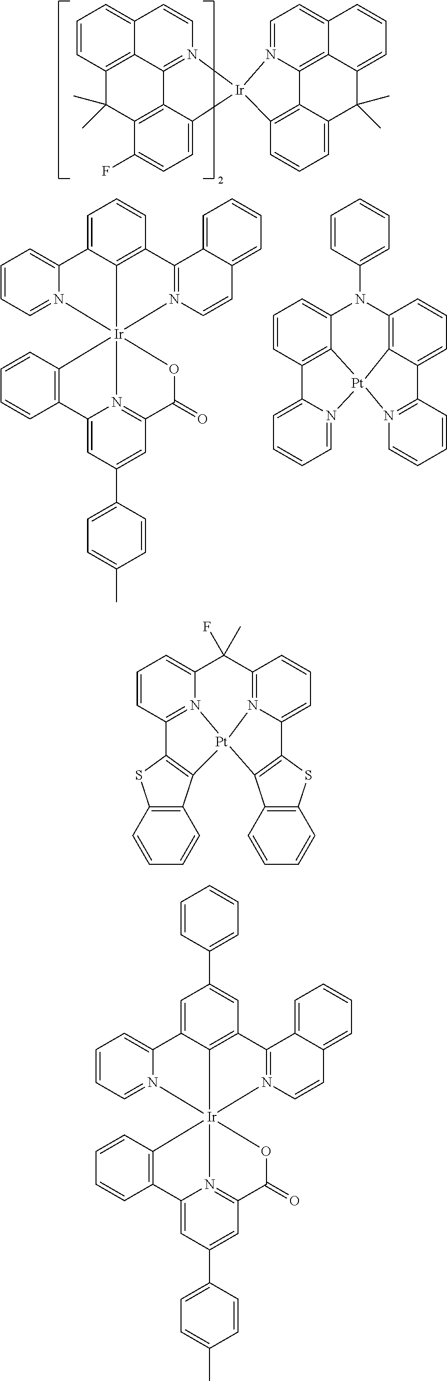

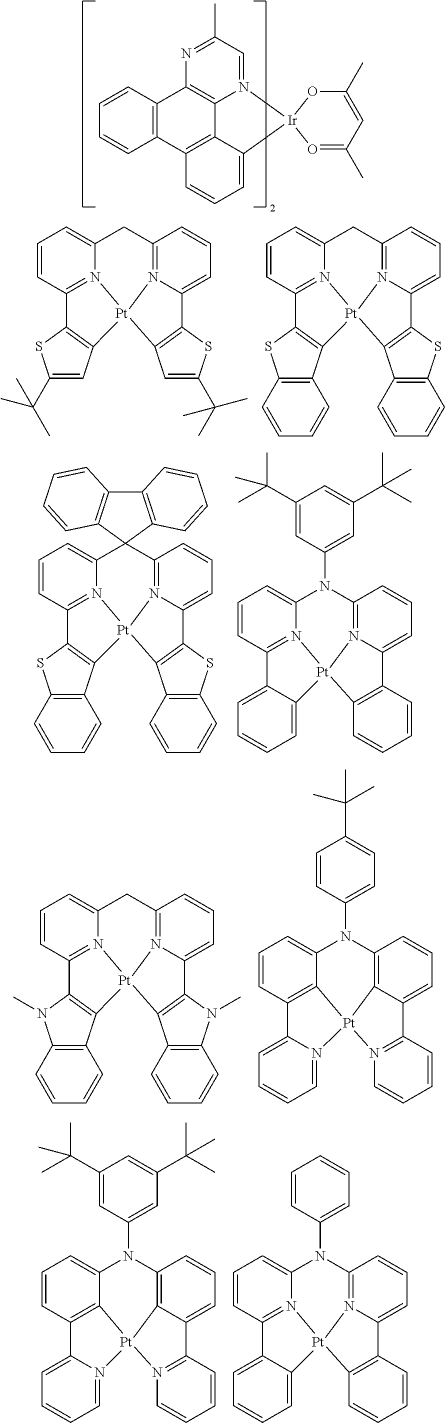

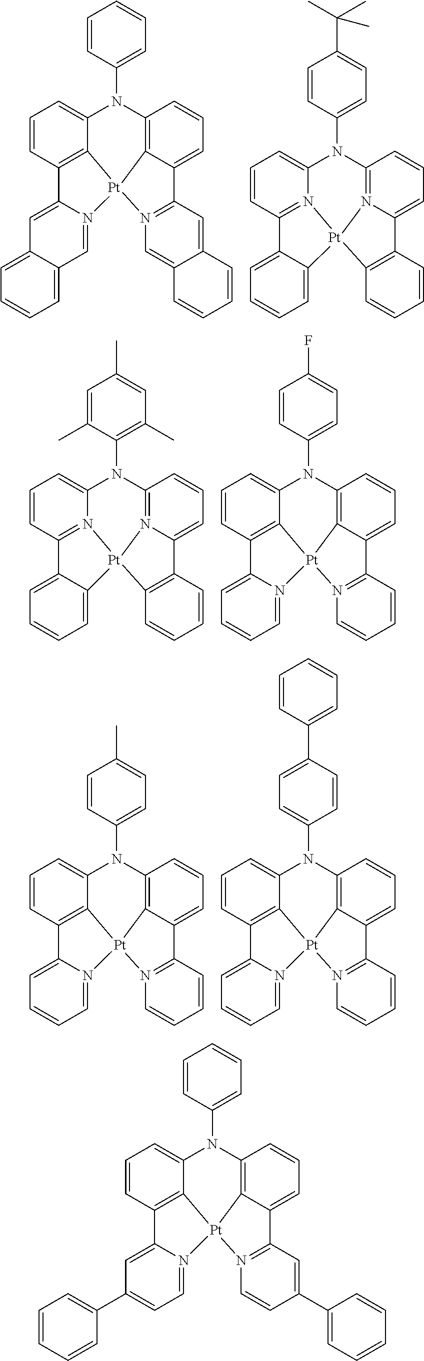

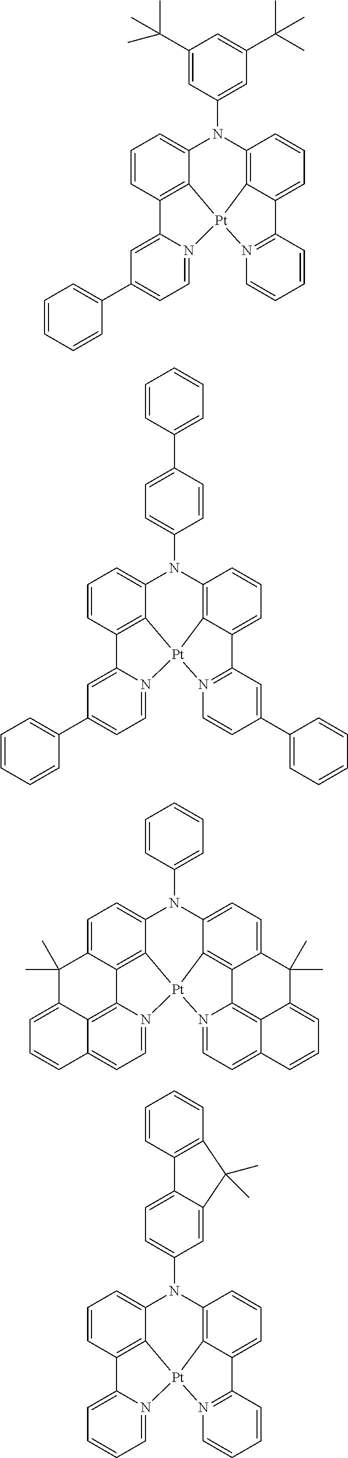

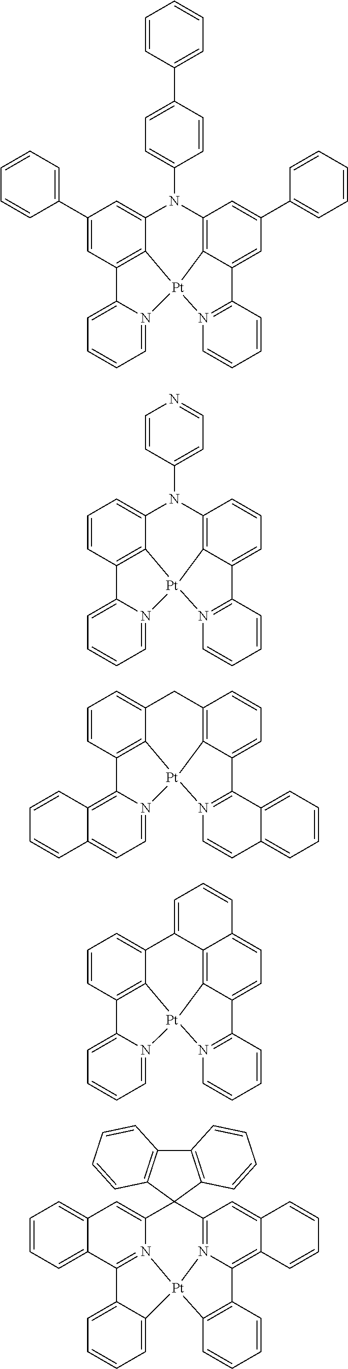

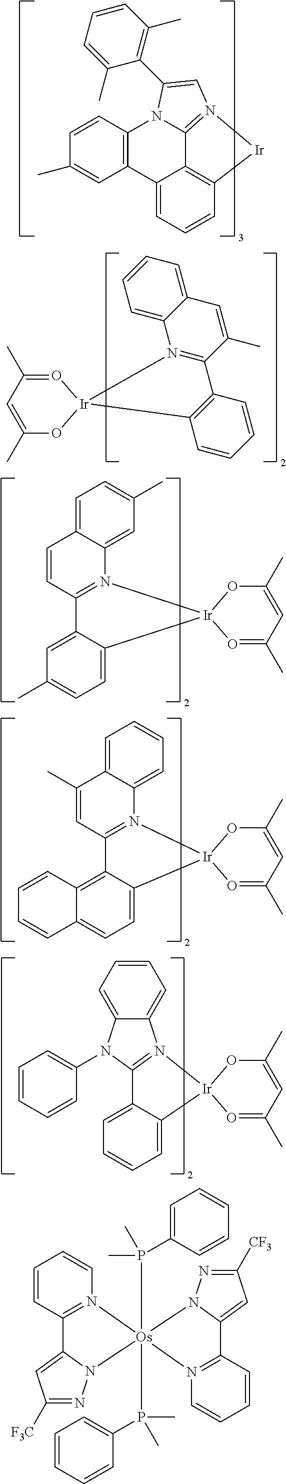

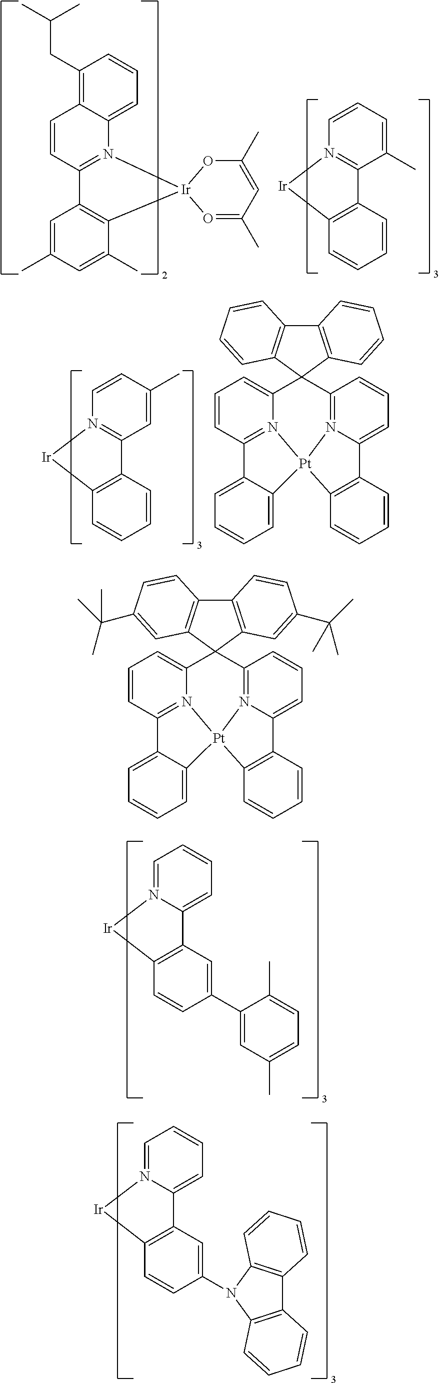

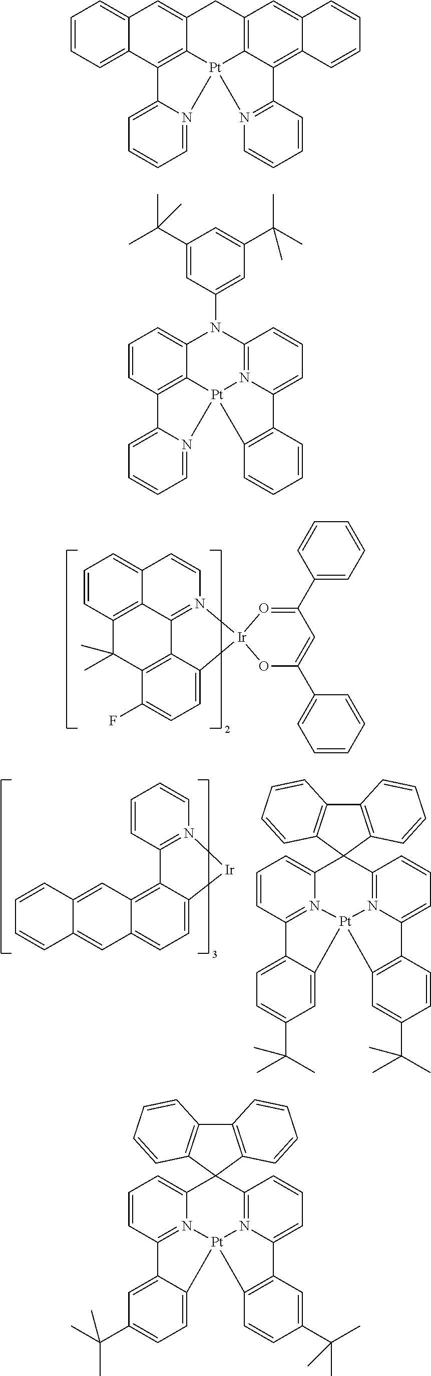

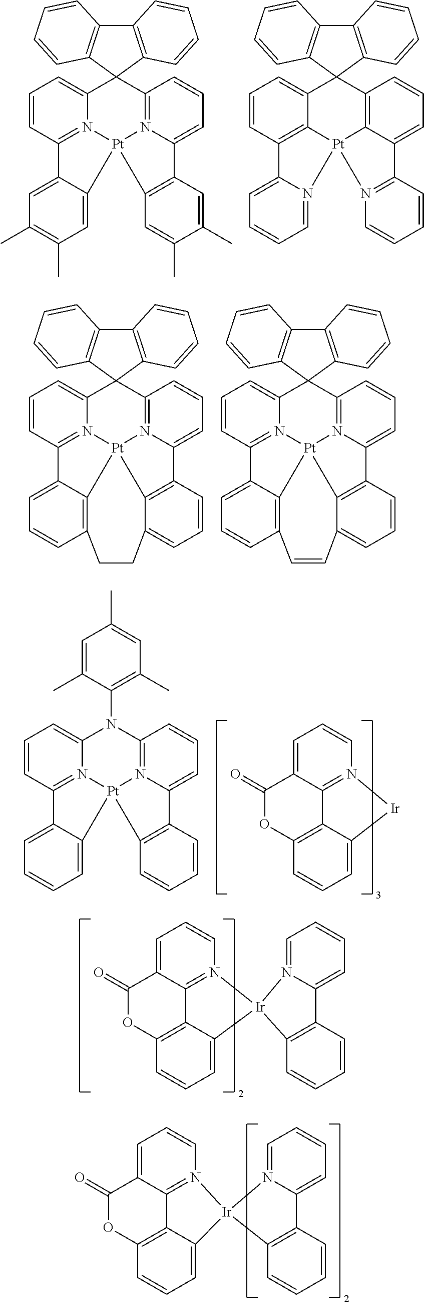

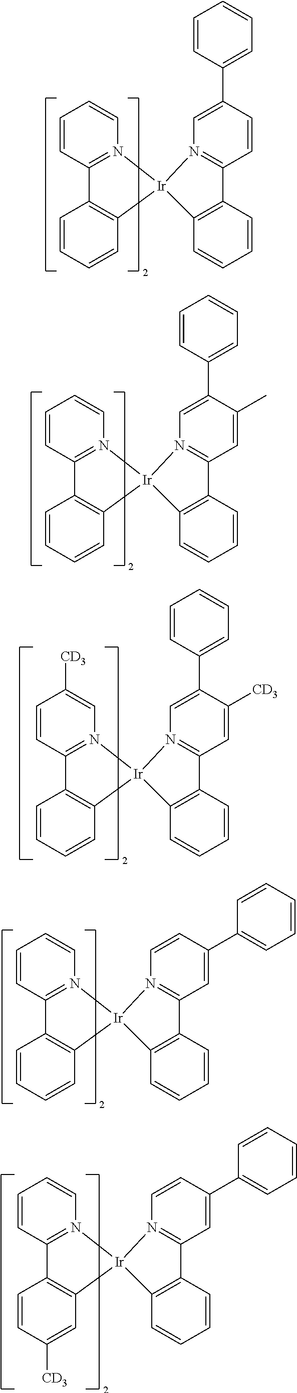

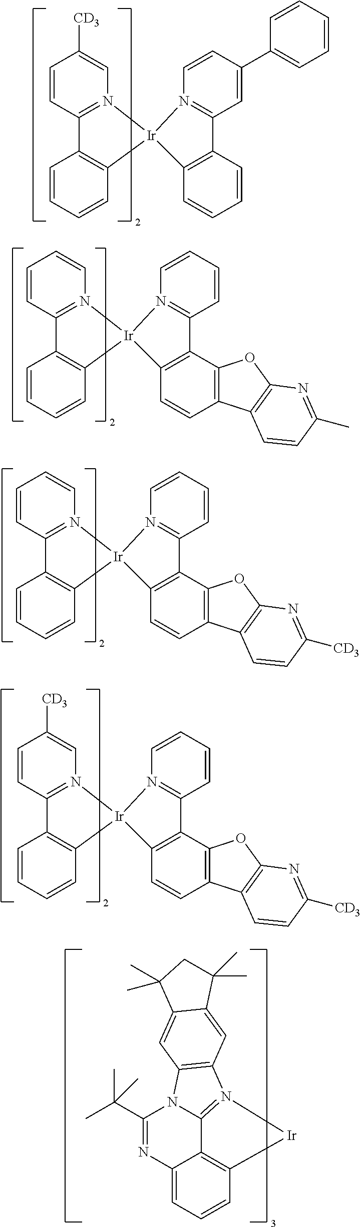

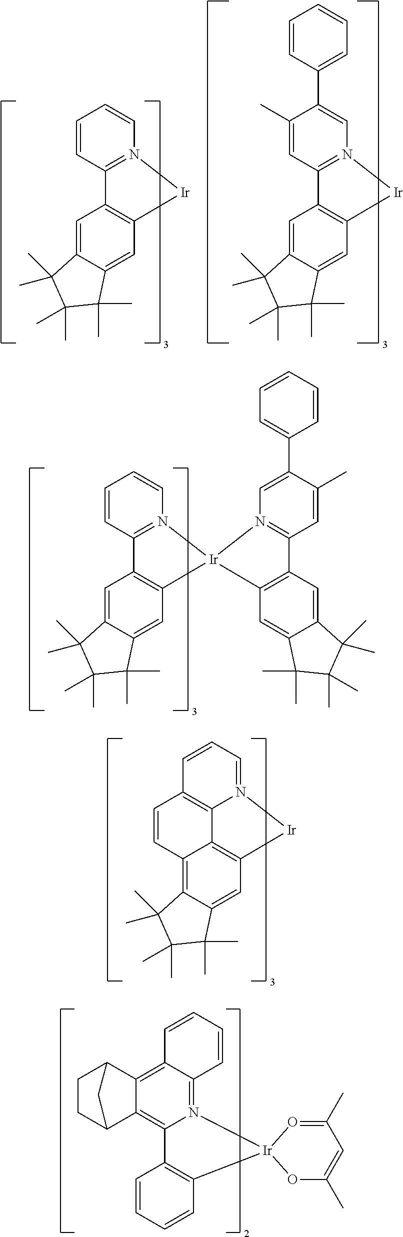

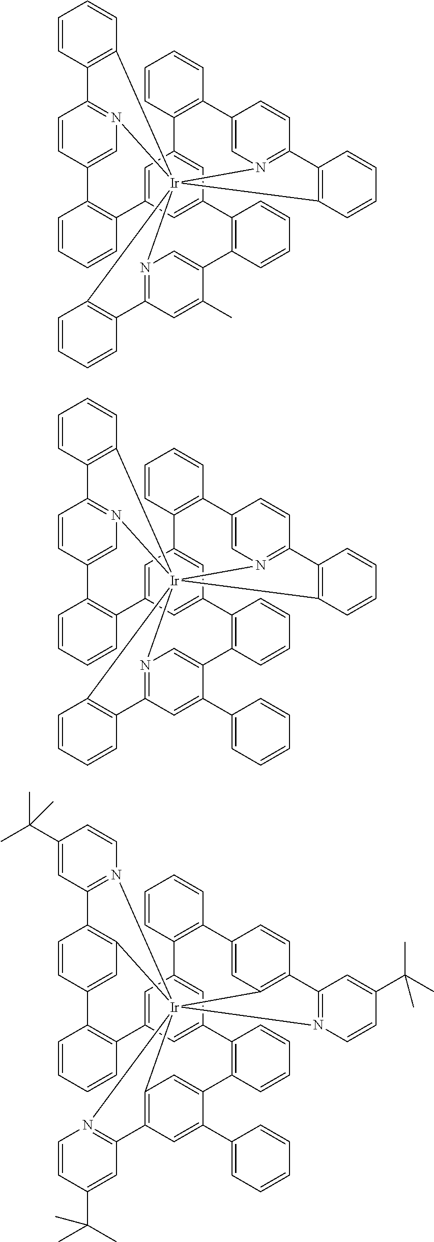

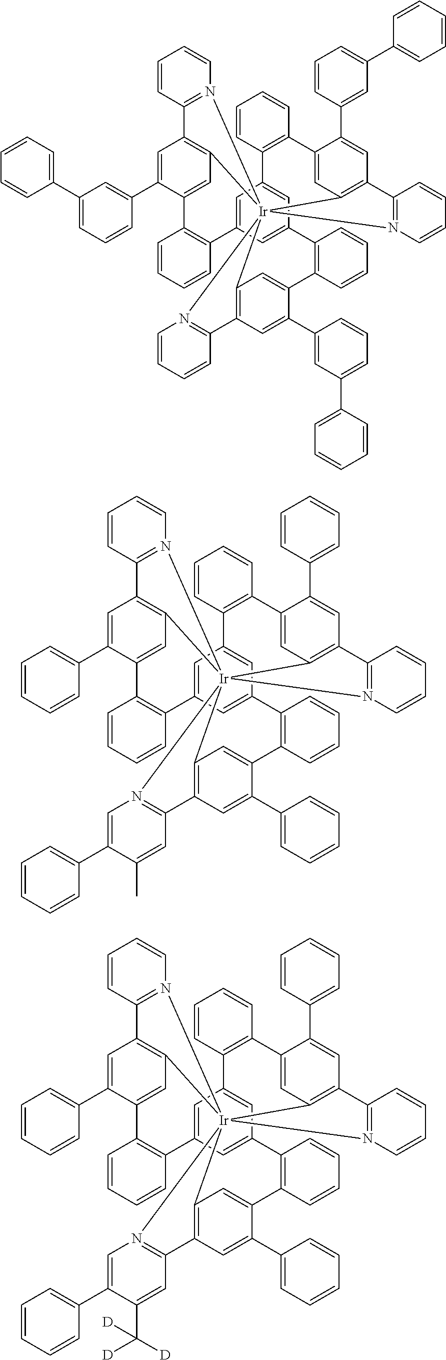

The compounds of the invention are especially also suitable as matrix materials for phosphorescent emitters in organic electroluminescent devices, as described, for example, in WO 98/24271, US 2011/0248247 and US 2012/0223633. In these multicolour display components, an additional blue emission layer is applied by vapour deposition over the full area to all pixels, including those having a colour other than blue.

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds of formula (1) or the above-recited preferred embodiments.

Additionally preferred is an organic electroluminescent device characterized in that one or more layers are coated by a sublimation process. In this case, the materials are applied by vapour deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapour phase deposition) method or with the aid of a carrier gas sublimation. In this case, the materials are applied at a pressure between 10⁻⁵ mbar and 1 bar. A special case of this method is the OVJP (organic vapour jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

In addition, hybrid methods are possible, in which, for example, one or more layers are applied from solution and one or more further layers are applied by vapour deposition.

These methods are known in general terms to those skilled in the art and can be applied by those skilled in the art without exercising inventive skill to organic electroluminescent devices comprising the compounds of the invention.

The compounds of the invention and the organic electroluminescent devices of the invention are notable for one or more of the following surprising advantages over the prior art:
1. The use of the compounds of the invention as matrix material for phosphorescent emitters leads to an improvement in lifetime. This is especially true with respect to use of lactams as matrix material, which have a similar structure to the compounds of the invention but do not contain a Y group.
2. The compounds of the invention lead to high efficiencies. This is especially true when the compounds are used as matrix material for a phosphorescent emitter.
3. The compounds of the invention lead to low operating voltages. This is especially true when the compounds are used as matrix material for a phosphorescent emitter.

The invention is illustrated in more detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

### Synthesis Examples

The syntheses which follow, unless stated otherwise, are conducted under a protective gas atmosphere in dried solvents. The solvents and reagents can be purchased, for example, from Sigma-ALDRICH or ABCR. The respective figures in square brackets or the numbers quoted for individual compounds relate to the CAS numbers of the compounds known from the literature.

### Preparation of the Synthons:

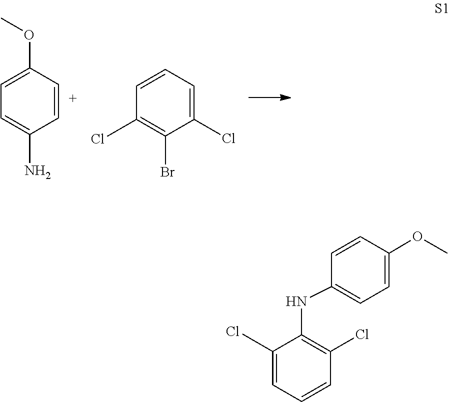

An initial charge of para-anisidine [104-94-9] (136.2 g, 1.11 mol), 1-bromo-2,6-dichlorobenzene [19393-92-1] (250.2 g, 1.11 mol) and sodium tert-butoxide (214.7 g, 2.23 mol) together with toluene (1500 ml) in a 4 l four-neck flask is inertized with argon for 30 min. Subsequently, Pd(dppf)Cl₂×DCM [95464-05-4] (4.51 g, 5.53 mmol) is added and the reaction mixture is stirred under reflux for 18 h. The mixture is then worked up by extraction with water and toluene, and the organic phase is dried over Na₂SO₄ and filtered through a silica gel bed. The filtrate is concentrated by rotary evaporation, and the crude product obtained is purified further by vacuum distillation. Yield: 218.3 g (813 mmol, 74%), yellow semicrystalline oily solid.

In an analogous manner, it is possible to prepare the following compounds: The catalyst system used here, rather than Pd(dppf)Cl₂×DCM, may also be Pd(OAc)₂/S-Phos [657408-07-6]. Purification can be effected not only by distillation but also using column chromatography, or recrystallization can be effected using standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methylpyrrolidone etc. The yields are typically in the range between 40% and 85%.

| Reactant 1 | Reactant 2 | Product |
|---|---|---|
| 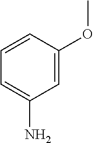 CAS-536-90-3 | 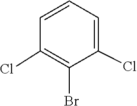 CAS-19393-92-1 | 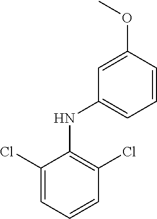 S2 |
| 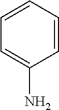 CAS-62-53-3 | 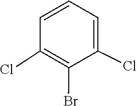 CAS-19393-92-1 | 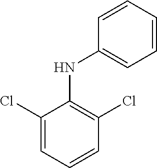 S3 |
| 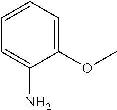 CAS-90-04-0 | 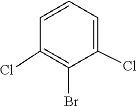 CAS-19393-92-1 | 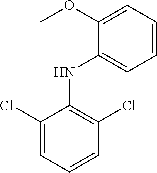 S4 |
| 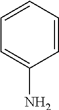 CAS-62-53-3 | 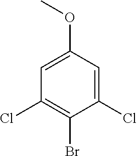 CAS-174913-20-3 | 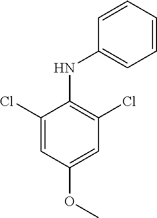 S5 |
| 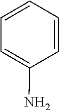 CAS-62-53-3 | 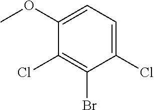 CAS-174913-16-7 | 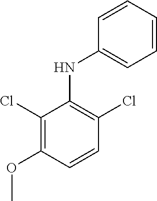 S6 |
| 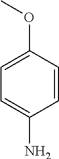 CAS-104-94-9 | 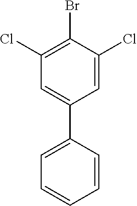 [SMILES: Clc1cc(-c2ccccc2)cc(Cl)c1Br] | 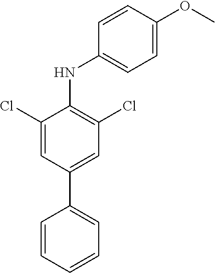 S7 |
| 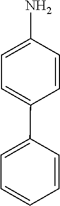 [SMILES: Nc1ccc(-c2ccccc2)cc1] CAS-92-67-1 | 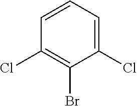 CAS-19393-92-1 | 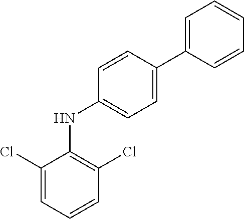 S8 |
| 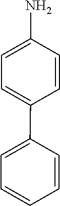 [SMILES: Nc1ccc(-c2ccccc2)cc1] CAS-92-67-1 | 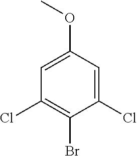 CAS-174913-20-3 | 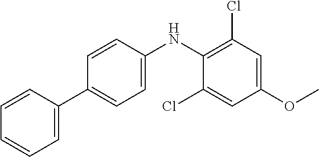 S9 |
| 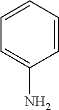 CAS-62-53-3 | 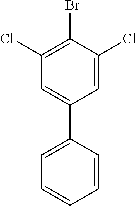 [SMILES: Clc1cc(-c2ccccc2)cc(Cl)c1Br] | 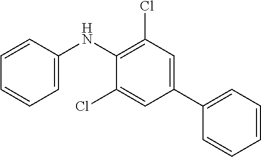 S10 |

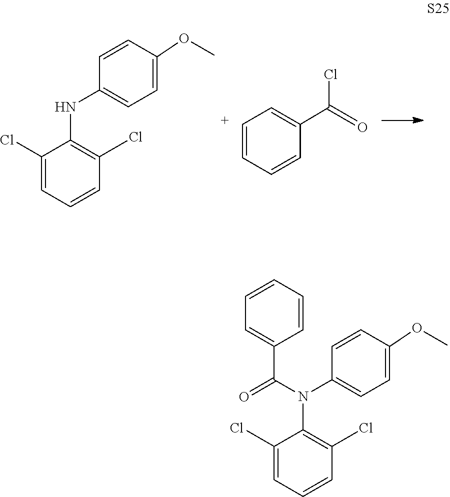 [SMILES: COc1ccc(Nc2c(Cl)cccc2Cl)cc1 ; O=C(Cl)c1ccccc1 ; COc1ccc(N(C(=O)c2ccccc2)c2c(Cl)cccc2Cl)cc1]

An initial charge of S1 (217.7 g, 812 mmol) in pyridine (1000 ml) is inertized with argon. Subsequently, benzoyl chloride [98-88-4] (150 ml, 1.29 mol) is added dropwise. After the addition has ended, the mixture is stirred under reflux for 15 h. The mixture is allowed to cool down to room temperature, and the precipitated solids are filtered off with suction and washed four times with water. The crude product is suspended in ethanol and stirred under reflux for 3 h. After cooling, the precipitated solids are filtered off with suction and washed with ethanol. Yield: 284 g (763 mmol, 94%) of white solid, 98% by ¹H NMR.

In an analogous manner, it is possible to prepare the following compounds: Purification can also be effected using column chromatography, or recrystallization can be effected using standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methylpyrrolidone etc. The yields are typically in the range between 40% and 85%.

| Reactant 1 | Reactant 2 | Product |
|---|---|---|
| 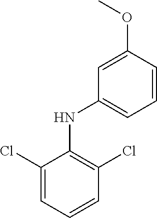 S2 | 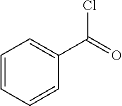 | 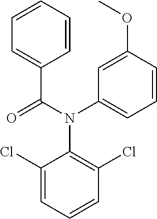 S26 |
| 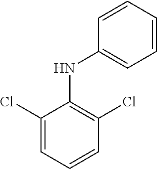 S3 | 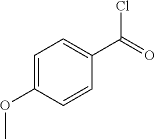 CAS-100-07-2 | 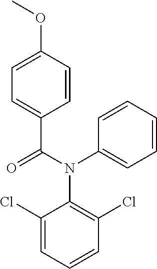 S27 |
| 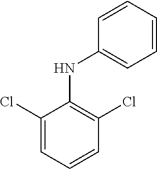 S3 | 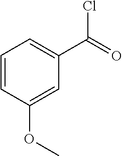 CAS-1711-05-3 | 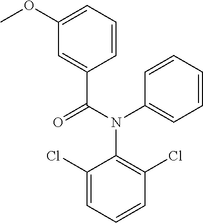 S28 |
| 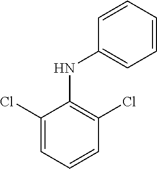 S3 | 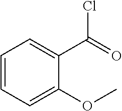 CAS-21615-34-9 | 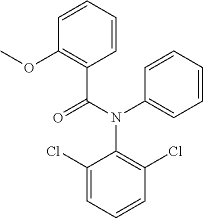 S29 |
| 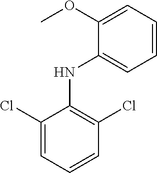 S4 | 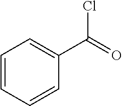 | 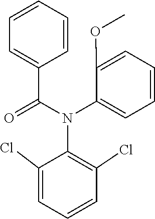 S30 |
| 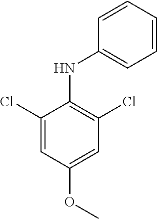 S5 | 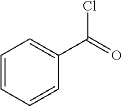 | 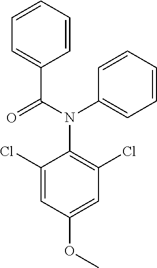 S31 |
| 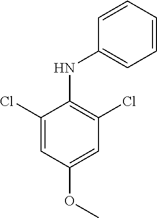 S5 | 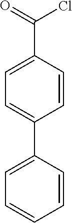 [SMILES: O=C(Cl)c1ccc(-c2ccccc2)cc1] CAS-14002-51-8 | 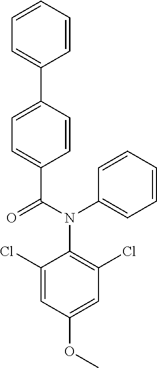 S32 |
| 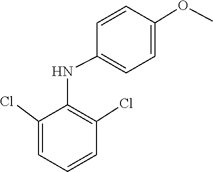 S1 | 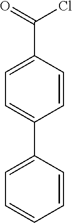 [SMILES: O=C(Cl)c1ccc(-c2ccccc2)cc1] CAS-14002-51-8 | 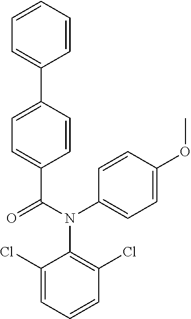 S33 |
| 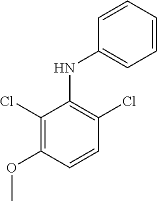 S6 | 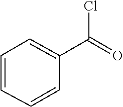 | 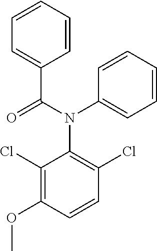 S34 |
| 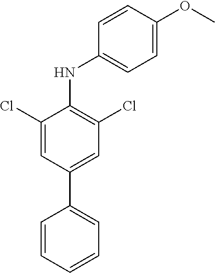 S7 | 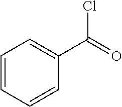 | 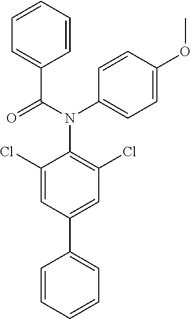 S35 |
| 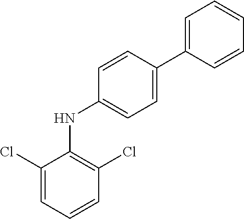 S8 | 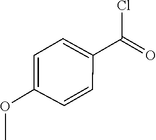 CAS-100-07-2 | 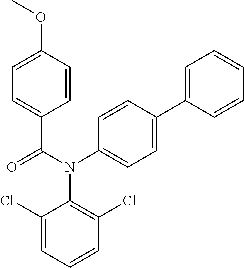 S36 |
| 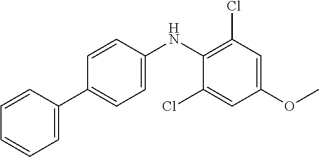 S9 | 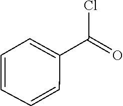 | 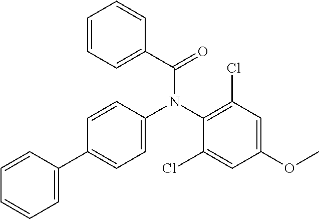 S37 |
| 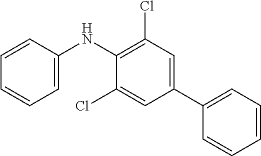 S10 | 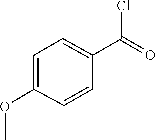 CAS-100-07-2 | 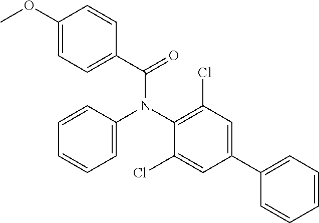 S38 |

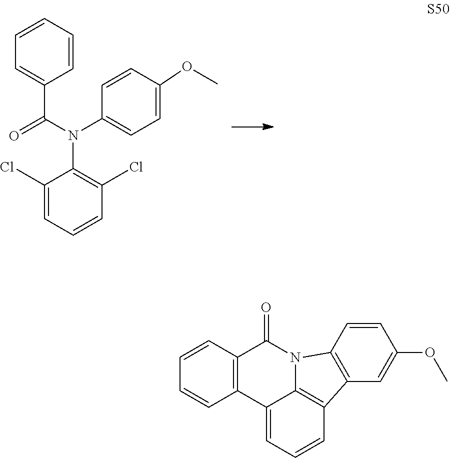 [SMILES: COc1ccc(N(C(=O)c2ccccc2)c2c(Cl)cccc2Cl)cc1 ; COc1ccc2c(c1)c1cccc3c4ccccc4c(=O)n2c31]

An initial charge of S25 (181.2 g, 486.8 mmol) and potassium carbonate (202.5 g, 1.46 mol) in N,N-dimethylacetamide (1800 ml) is inertized with argon for 30 min. Subsequently, 1,3-bis(2,6-diisopropylphenyl)imidazolium chloride [CAS-250285-32-6] (8.55 g, 19.47 mmol) and palladium(II) acetate (2.18 g, 9.69 mmol) are added, and the reaction mixture is heated to reflux and stirred at this temperature for 72 h. After cooling to room temperature, the solvent is drawn off on a rotary evaporator, the residue is stirred with ethanol/water (1:1; 1000 ml), and the solids are filtered off with suction and washed with water and ethanol. The crude product is recrystallized from ethyl acetate. Yield: 49.4 g (165 mmol, 34%) of brown solid; about 95% by ¹H NMR.

In an analogous manner, it is possible to prepare the following compounds: The ligand used here, rather than 1,3-bis(2,6-diisopropylphenyl)imidazolium chloride [CAS-250285-32-6], may also be 1,3-bis(2,4,6-trimethylphenyl)imidazolium chloride [141556-45-8], tricyclohexylphosphine [2622-14-2] or tri-tert-butylphosphine [13716-12-6]. Purification can be effected not only by distillation but also using column chromatography, or recrystallization can be effected using standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methylpyrrolidone etc. The yields are typically in the range between 10% and 50%.

| Reactant | Product(s) |
|---|---|
| 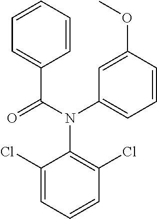 S26 | 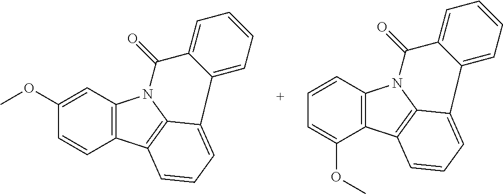 [SMILES: COc1ccc2c3cccc4c5ccccc5c(=O)n(c2c1)c43 ; COc1cccc2c1c1cccc3c4ccccc4c(=O)n2c31] S51 S52 Separation of the isomers via column chromatography |
| 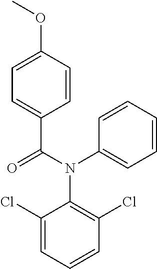 S27 | 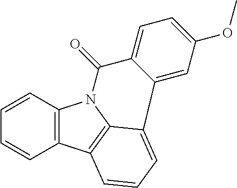 [SMILES: COc1ccc2c(=O)n3c4ccccc4c4cccc(c2c1)c43] S53 |
| 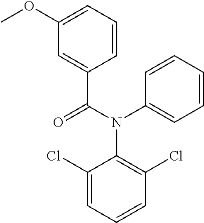 S28 | 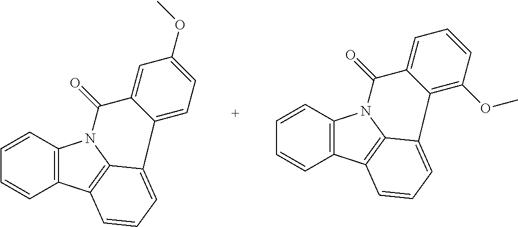 [SMILES: COc1ccc2c(c1)c(=O)n1c3ccccc3c3cccc2c31 ; COc1cccc2c(=O)n3c4ccccc4c4cccc(c12)c43] S54 S55 Separation of the isomers via column chromatography |
| 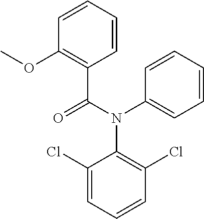 S29 | 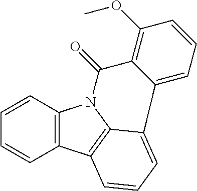 [SMILES: COc1cccc2c1c(=O)n1c3ccccc3c3cccc2c31] S56 |
| 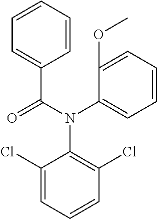 S30 | 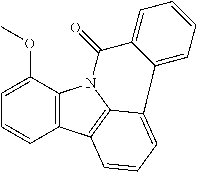 [SMILES: COc1cccc2c3cccc4c5ccccc5c(=O)n(c12)c43] S57 |
| 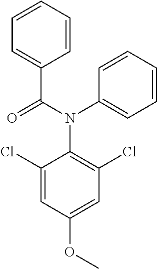 S31 | 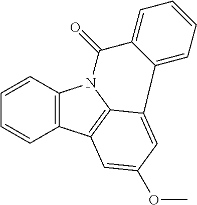 [SMILES: COc1cc2c3ccccc3c(=O)n3c4ccccc4c(c1)c23] S58 |
| 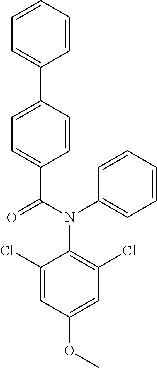 S32 | 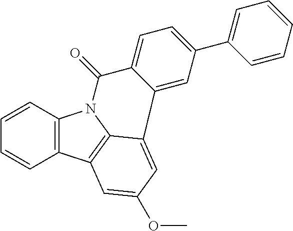 [SMILES: COc1cc2c3cc(-c4ccccc4)ccc3c(=O)n3c4ccccc4c(c1)c23] S59 |
| 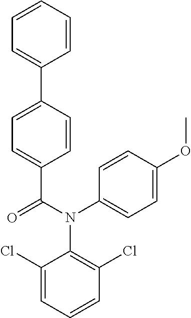 S33 | 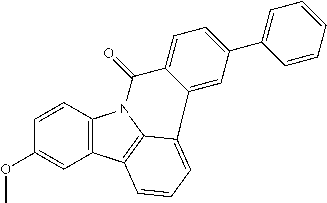 [SMILES: COc1ccc2c(c1)c1cccc3c4cc(-c5ccccc5)ccc4c(=O)n2c31] S60 |
| 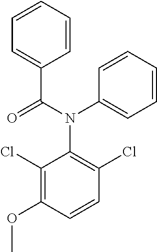 S34 | 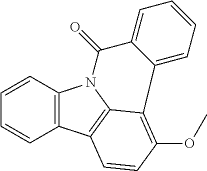 [SMILES: COc1ccc2c3ccccc3n3c(=O)c4ccccc4c1c23] S61 |
| 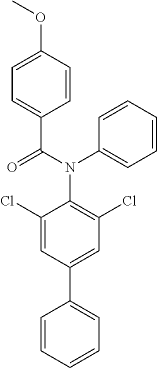 S38 | 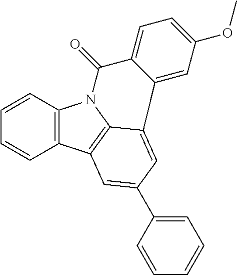 [SMILES: COc1ccc2c(=O)n3c4ccccc4c4cc(-c5ccccc5)cc(c2c1)c43] S62 |
| 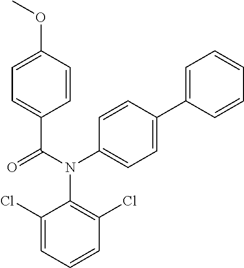 S36 | 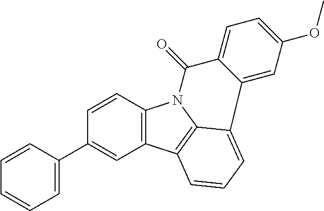 [SMILES: COc1ccc2c(=O)n3c4ccc(-c5ccccc5)cc4c4cccc(c2c1)c43] S63 |
| 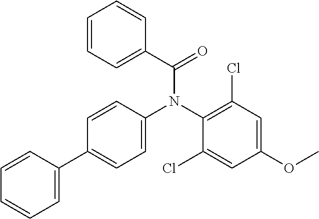 S37 | 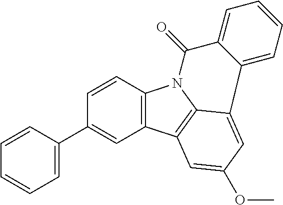 [SMILES: COc1cc2c3ccccc3c(=O)n3c4ccc(-c5ccccc5)cc4c(c1)c23] S64 |
| 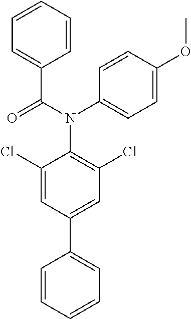 S35 | 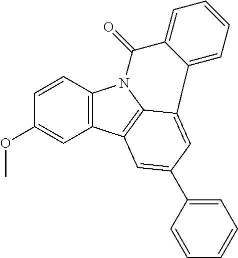 [SMILES: COc1ccc2c(c1)c1cc(-c3ccccc3)cc3c4ccccc4c(=O)n2c31] S65 |

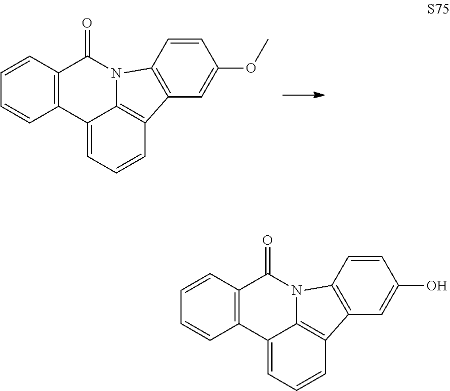 [SMILES: COc1ccc2c(c1)c1cccc3c4ccccc4c(=O)n2c31 ; O=c1c2ccccc2c2cccc3c4cc(O)ccc4n1c23]

An initial charge of S50 (36.84 g, 123.1 mmol) in a flask under an argon atmosphere in dichloromethane (1200 ml) is cooled in an ice bath to 000. Subsequently, boron tribromide [10294-33-4] (24.0 ml, 252.9 mmol) is added dropwise, and then the mixture is gradually warmed up to room temperature. Subsequently, the mixture is quenched by dropwise addition of methanol and the solvent is drawn off on a rotary evaporator. Methanol (300 ml) is twice added and removed again by rotary evaporation. The brown solids are admixed with 400 ml of methanol and heated under reflux. After cooling, the solids are filtered off with suction and washed with methanol. The crude product is subjected to hot extraction with n-butyl acetate.

Yield: 28.5 g (100 mmol; 81%) of beige solid; 95% by ¹H NMR.

In an analogous manner, it is possible to prepare the following compounds: Purification can also be effected using column chromatography, or recrystallization can be effected using standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methylpyrrolidone etc. The yields are typically in the range between 50% and 90%.

| Reactant | Product(s) |
|---|---|
| 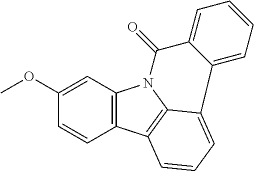 S51 | 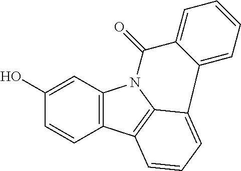 S76 |
| 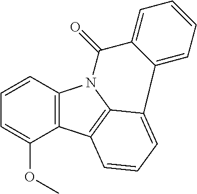 S52 | 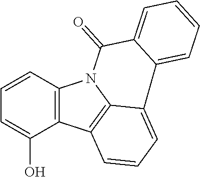 S77 |
| 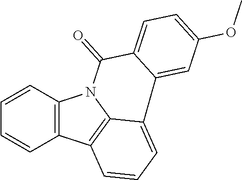 S53 | 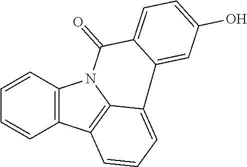 S78 |
| 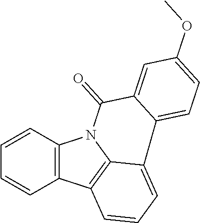 S54 | 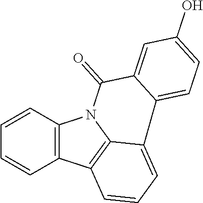 S79 |
| 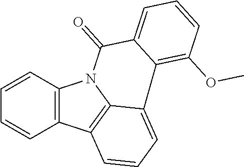 S55 | 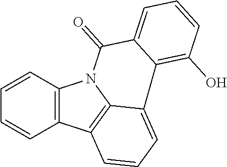 S80 |
| 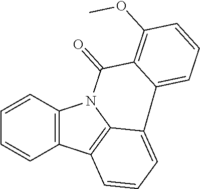 S56 | 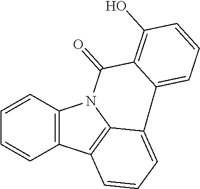 S81 |
| 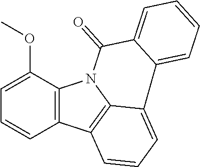 S57 | 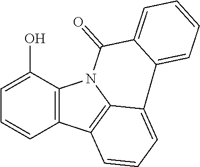 S82 |
| 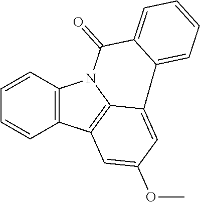 S58 | 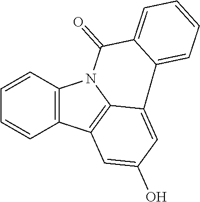 S83 |
| 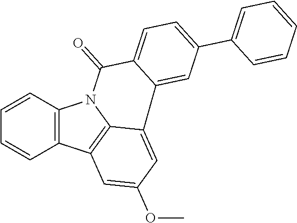 S59 | 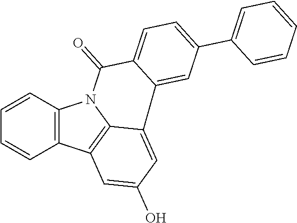 S84 |
| 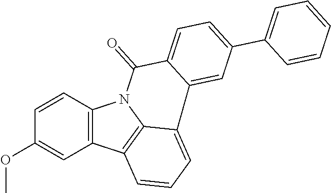 S60 | 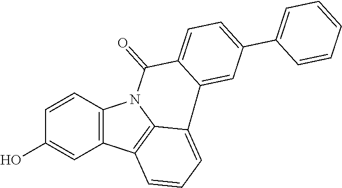 S85 |
| 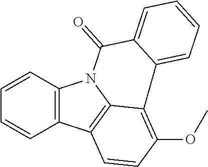 S61 | 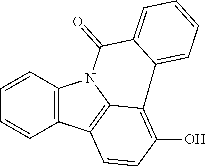 S86 |
| 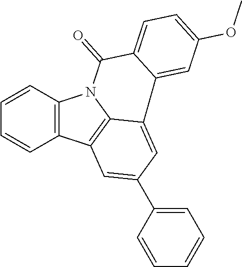 S62 | 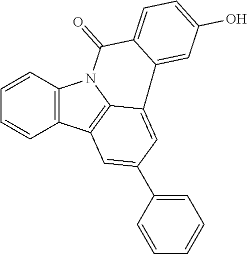 S87 |
| 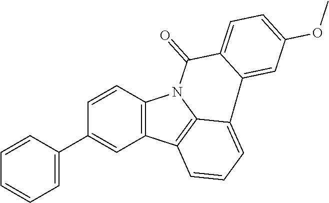 S63 | 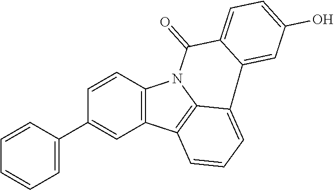 S88 |
| 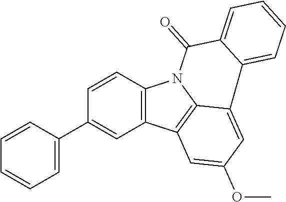 S64 | 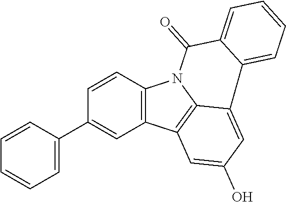 S89 |
| 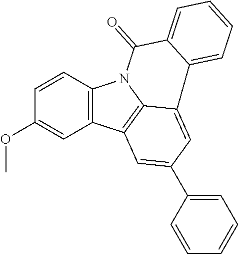 S65 | 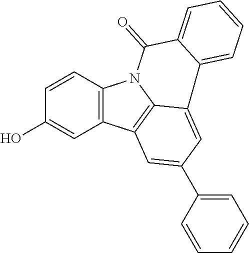 S90 |

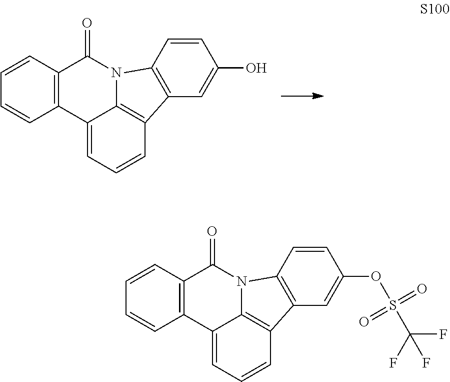 [SMILES: O=c1c2ccccc2c2cccc3c4cc(O)ccc4n1c23 ; O=c1c2ccccc2c2cccc3c4cc(OS(=O)(=O)C(F)(F)F)ccc4n1c23]

An initial charge of S75 (27.84 g, 97.6 mmol) in pyridine (500 ml) is cooled in an ice bath to 0° C. Subsequently, trifluoromethanesulfonic anhydride [358-23-6] (50.4 ml, 293.4 mmol) is gradually added dropwise at such a rate that the internal temperature does not rise above 10° C. Subsequently, the mixture is left to warm up to room temperature overnight. The pyridine is drawn off on a rotary evaporator and the residue is worked up by extraction with dichloromethane and 1 mol/I HCl. The organic phase is washed four times with water, dried over Na₂SO₄ and concentrated to 300 ml. The precipitated solids are filtered off with suction and washed with dichloromethane and ethanol. Yield: 38.7 g (92.7 mmol, 95%) of beige solid, 95% by ¹H NMR.

In an analogous manner, it is possible to prepare the following compounds: Purification can also be effected using column chromatography, or recrystallization can be effected using standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methylpyrrolidone etc. The yields are typically in the range between 55% and 97%.

| Reactant | Product(s) |
|---|---|
| 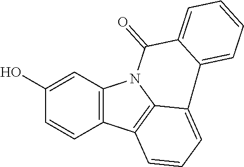 S76 | 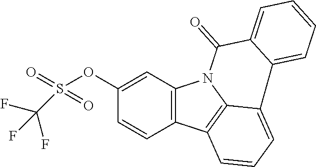 S101 |
| 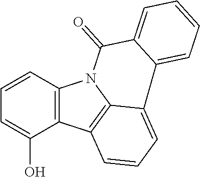 S77 | 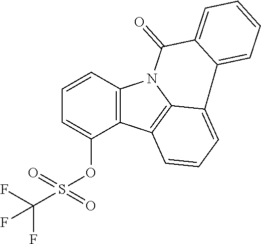 S102 |
| 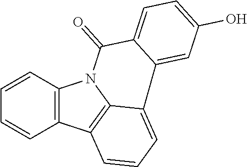 S78 | 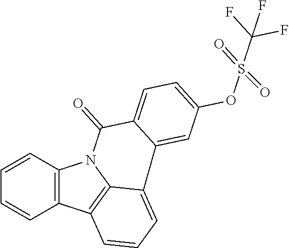 S103 |
| 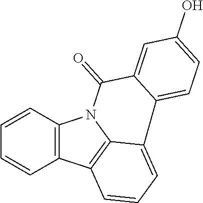 S79 | 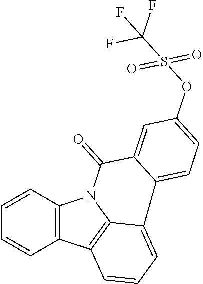 S104 |
| 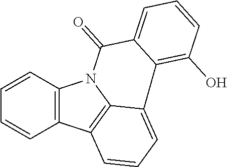 S80 | 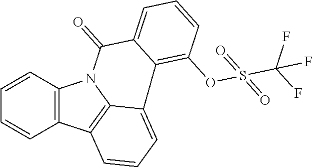 S105 |
| 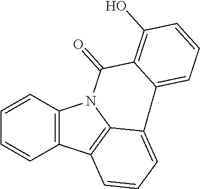 S81 | 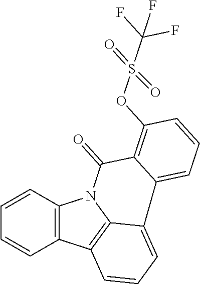 S106 |
| 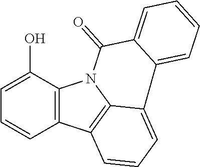 S82 | 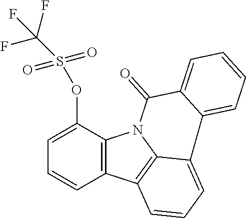 S107 |
| 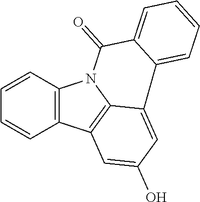 S83 | 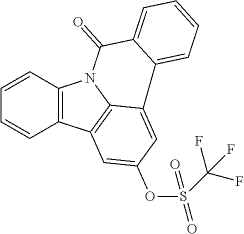 S108 |
| 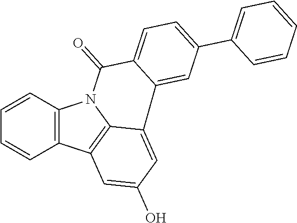 S84 | 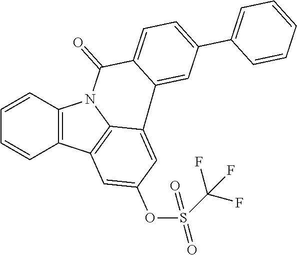 S109 |
| 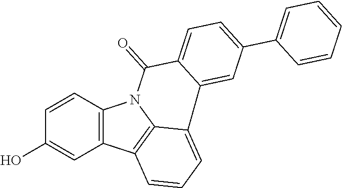 S85 | 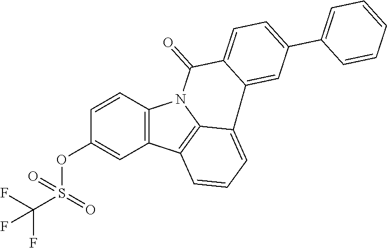 S110 |
| 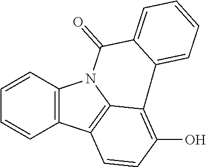 S86 | 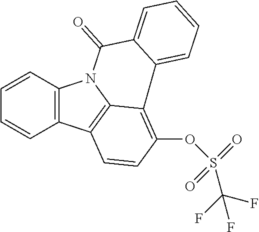 S111 |
| 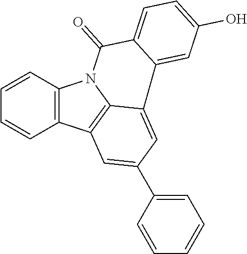 S87 | 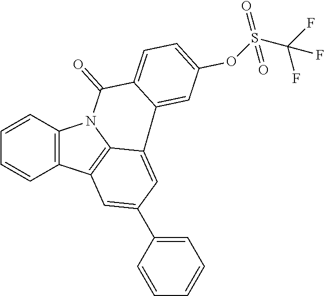 S112 |
| 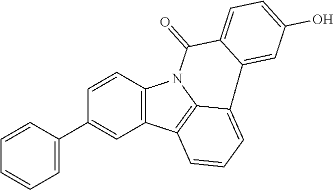 S88 | 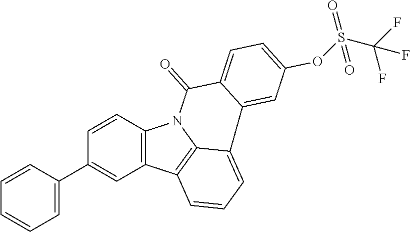 S113 |
| 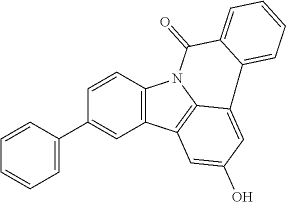 S89 | 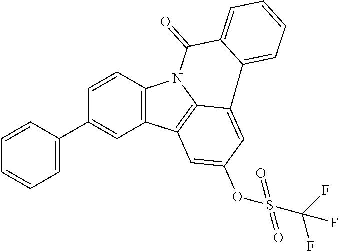 S114 |
| 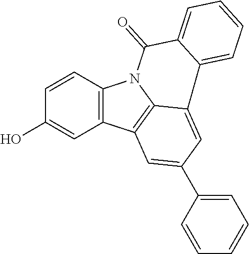 S90 | 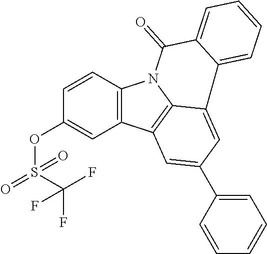 S115 |

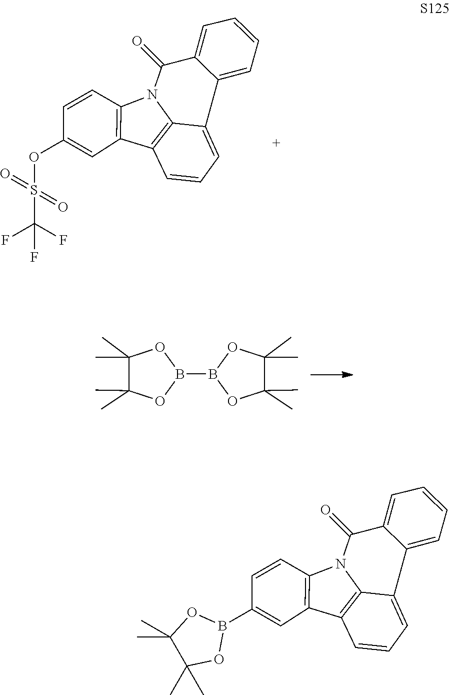

An initial charge of S1 (40.2 g, 96.2 mmol), bis(pinacolato)diboron [73183-34-3] (26.93 g, 105.0 mmol) and potassium acetate (28.82 g, 293.6 mmol) in 1,4-dioxane (600 ml) is inertized with argon for 2 min. Subsequently, X-Phos [564483-18-7] (456 mg, 0.96 mmol) and Pd₂(dba)₃ [51364-51-3](435 mg, 0.48 mmol) are added and the reaction mixture is stirred under reflux for 16 h. After cooling, the solvent is removed by rotary evaporation and the residue is worked up by extraction with dichloromethane/water. The organic phase is dried over Na₂SO₄, ethyl acetate is added, and the dichloromethane is removed by rotary evaporation on a rotary evaporator down to 500 mbar. The precipitated solids are filtered off with suction and washed with ethyl acetate. Yield: 30.8 g (77.9 mmol, 81%) of beige solid, 98% by ¹H NMR.

In an analogous manner, it is possible to prepare the following compounds: The ligand used here may also be S-Phos rather than X-Phos. Purification can be effected using column chromatography, or recrystallization can be effected using standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methylpyrrolidone etc. The yields are typically in the range between 60% and 95%.

| Reactant | Product(s) |
|---|---|
| 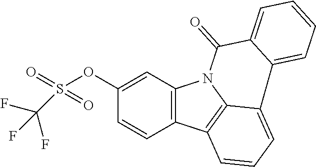 S101 | 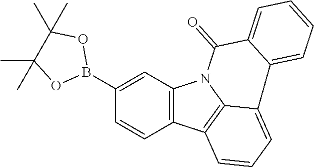 S126 |
| 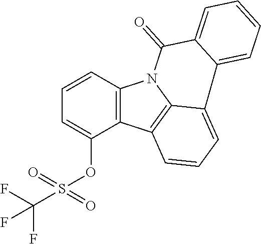 S102 | 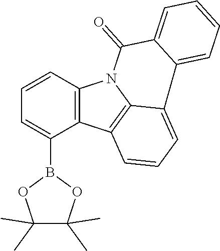 S127 |
| 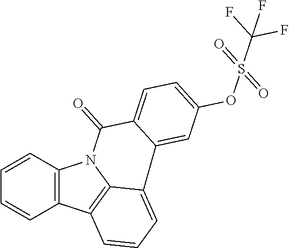 S103 | 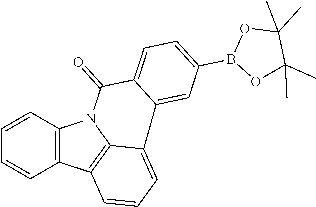 S128 |
| 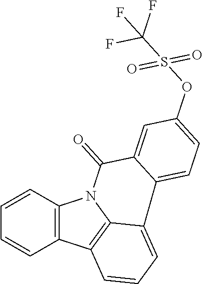 S104 | 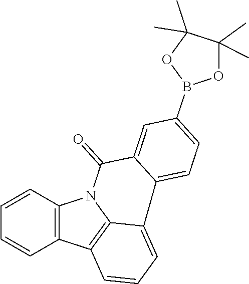 S129 |
| 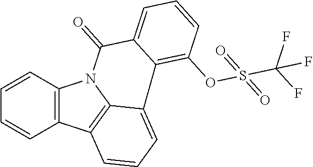 S105 | 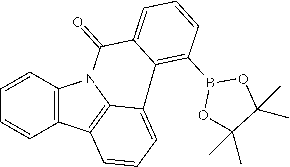 S130 |
| 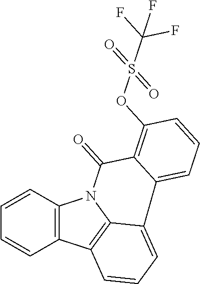 S106 | 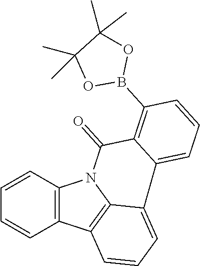 S131 |
| 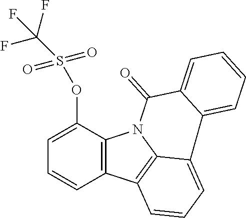 S107 | 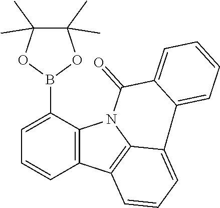 S132 |
| 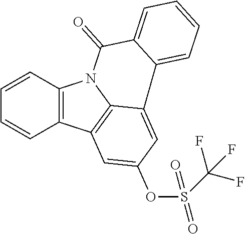 S108 | 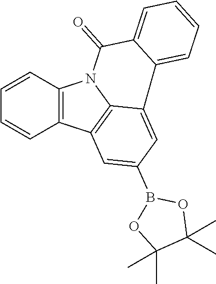 S133 |
| 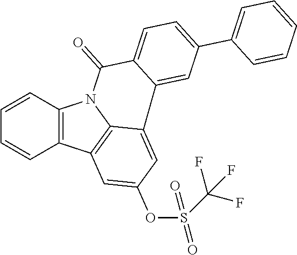 S109 | 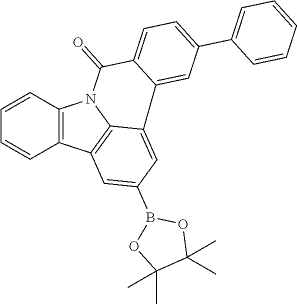 S134 |
| 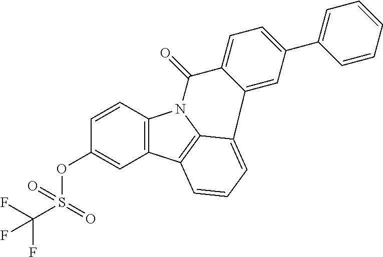 S110 | 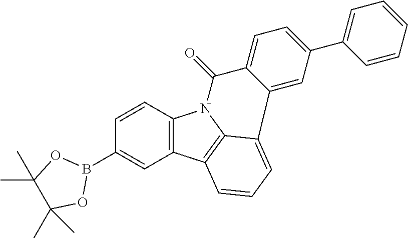 S135 |
| 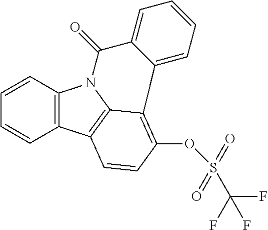 S111 | 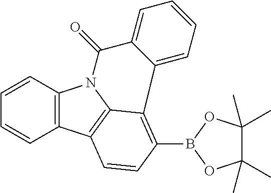 S136 |
| 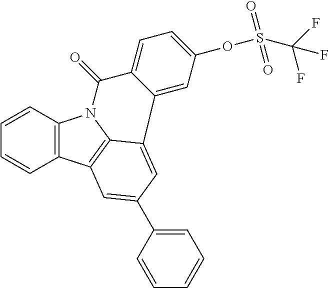 S112 | 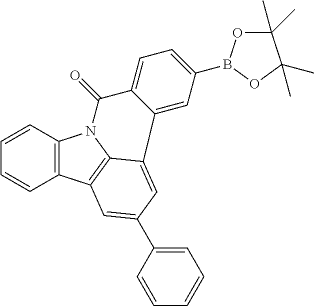 S137 |
| 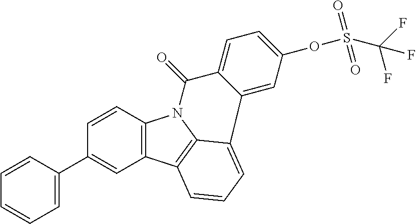 S113 | 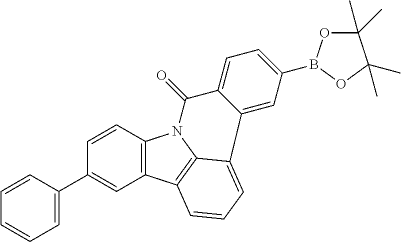 S138 |
| 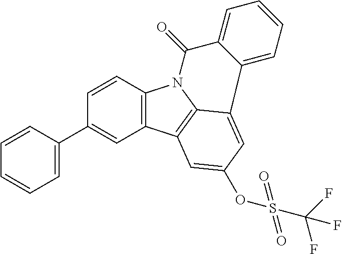 S114 | 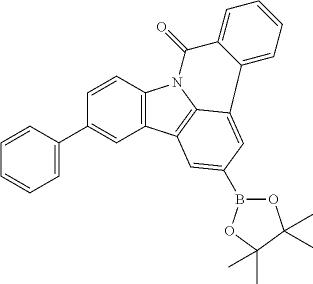 S139 |
| 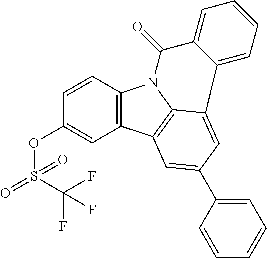 S115 | 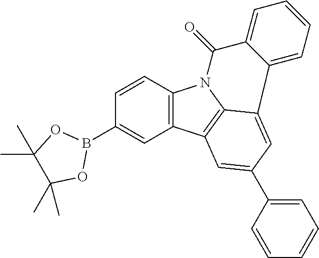 S140 |

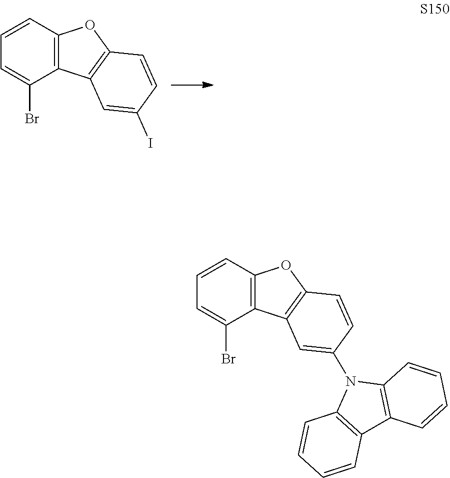 [SMILES: Brc1cccc2oc3ccc(I)cc3c12 ; Brc1cccc2oc3ccc(-n4c5ccccc5c5ccccc54)cc3c12]

Under an inert atmosphere, an initial charge of 1-bromo-8-iododibenzofuran [CAS-1822311-11-4] (37.28 g, 100 mmol), carbazole [86-74-8] (16.71 g, 100 mmol) of potassium carbonate (34.55 g, 250 mmol) and copper powder (1.27 g, 20.0 mmol) in DMF (350 ml) is inertized with argon for a further 15 min and then stirred at 130° C. for 32 h. The mixture is left to cool down to room temperature, filtered through a Celite bed and washed through twice with 200 ml of DMF, and the filtrate is concentrated to dryness on a rotary evaporator. The residue is worked up by extraction with dichloromethane/water, and the organic phase is washed twice with water and once with saturated NaCl solution and dried over Na₂SO₄. 150 ml of ethanol are added, dichloromethane is drawn off on a rotary evaporator down to 500 mbar, and the precipitated solids are filtered off with suction and washed with ethanol. Yield: 29.2 g (71.1 mmol, 71%) of grey solid, 97% by ¹H NMR.

In an analogous manner, it is possible to prepare the following compounds: Purification can be effected using column chromatography, or recrystallization can be effected using standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methylpyrrolidone etc. The yields are typically in the range between 20% and 80%.

| Reactant 1 | Reactant 2 | Product |
|---|---|---|
| 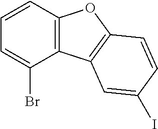 [SMILES: Brc1cccc2oc3ccc(I)cc3c12] CAS-1822311-11-4 | 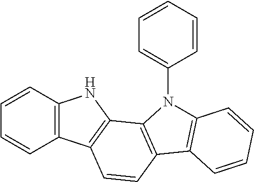 [SMILES: c1ccc(-n2c3ccccc3c3ccc4c5ccccc5[nH]c4c32)cc1] CAS-1024598-06-8 | 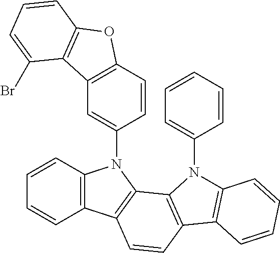 S151 |
| 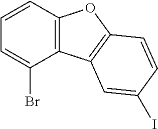 [SMILES: Brc1cccc2oc3ccc(I)cc3c12] CAS-1822311-11-4 | 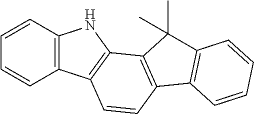 [SMILES: CC1(C)c2ccccc2-c2ccc3c([nH]c4ccccc43)c21] CAS-1329054-41-2 | 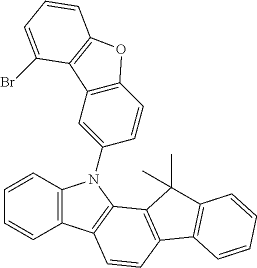 S152 |
| 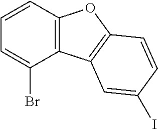 [SMILES: Brc1cccc2oc3ccc(I)cc3c12] CAS-1822311-11-4 | 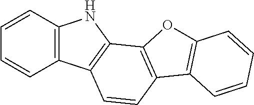 [SMILES: c1ccc2c(c1)[nH]c1c2ccc2c3ccccc3oc21] CAS-1338919-70-2 | 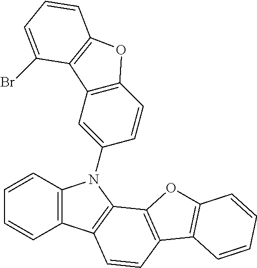 S153 |
| 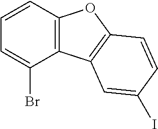 [SMILES: Brc1cccc2oc3ccc(I)cc3c12] CAS-1822311-11-4 | 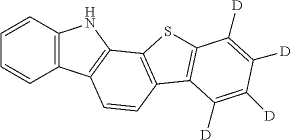 [SMILES: [2H]c1c([2H])c([2H])c2c(sc3c2ccc2c4ccccc4[nH]c23)c1[2H]] CAS-1447708-61-3 | 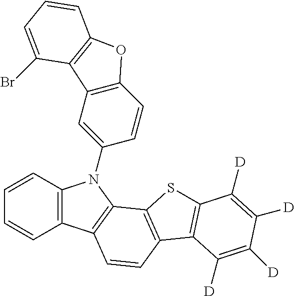 S154 |
| 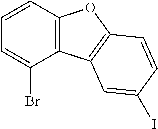 [SMILES: Brc1cccc2oc3ccc(I)cc3c12] CAS-1822311-11-4 | 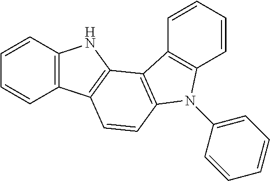 [SMILES: c1ccc(-n2c3ccccc3c3c4[nH]c5ccccc5c4ccc32)cc1] CAS-1247053-55-9 | 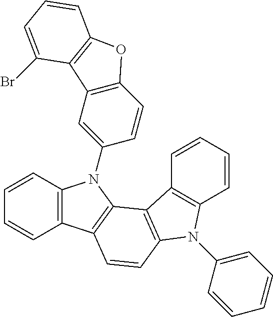 S155 |
| 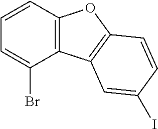 [SMILES: Brc1cccc2oc3ccc(I)cc3c12] CAS-1822311-11-4 | 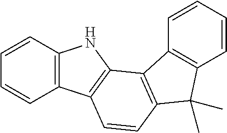 [SMILES: CC1(C)c2ccccc2-c2c1ccc1c2[nH]c2ccccc21] CAS-1616231-39-0 | 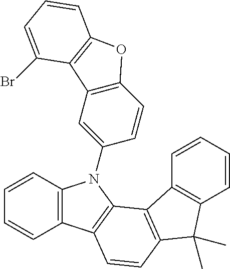 S156 |
| 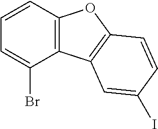 [SMILES: Brc1cccc2oc3ccc(I)cc3c12] CAS-1822311-11-4 | 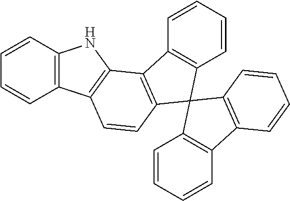 [SMILES: c1ccc2c(c1)-c1ccccc1C21c2ccccc2-c2c1ccc1c2[nH]c2ccccc21] CAS-1615703-28-0 | 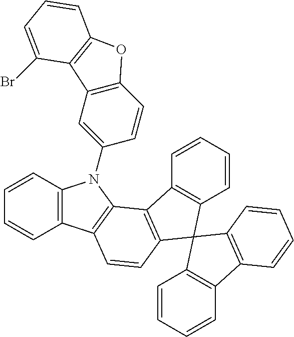 S157 |
| 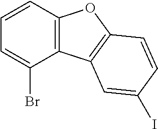 [SMILES: Brc1cccc2oc3ccc(I)cc3c12] CAS-1822311-11-4 | 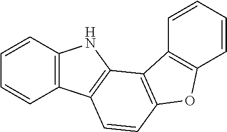 [SMILES: c1ccc2c(c1)[nH]c1c2ccc2oc3ccccc3c21] CAS-1246308-85-9 | 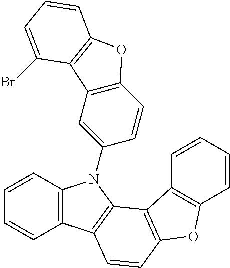 S158 |
| 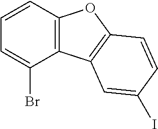 [SMILES: Brc1cccc2oc3ccc(I)cc3c12] CAS-1822311-11-4 | 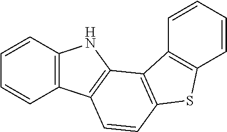 [SMILES: c1ccc2c(c1)[nH]c1c2ccc2sc3ccccc3c21] CAS-206447-68-9 | 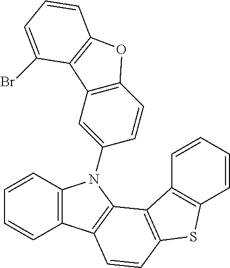 S159 |
| 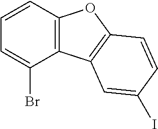 [SMILES: Brc1cccc2oc3ccc(I)cc3c12] CAS-1822311-11-4 | 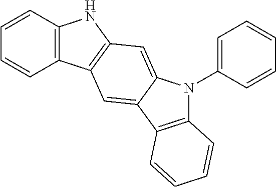 [SMILES: c1ccc(-n2c3ccccc3c3cc4c(cc32)[nH]c2ccccc24)cc1] CAS-1448296-00-1 | 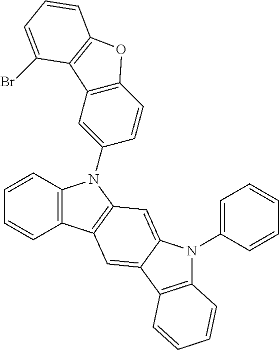 S160 |
| 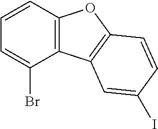 [SMILES: Brc1cccc2oc3ccc(I)cc3c12] CAS-1822311-11-4 | 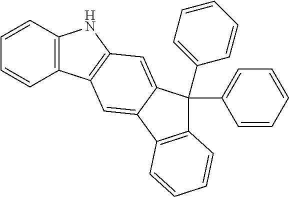 [SMILES: c1ccc(C2(c3ccccc3)c3ccccc3-c3cc4c(cc32)[nH]c2ccccc24)cc1] CAS-1257220-52-2 | 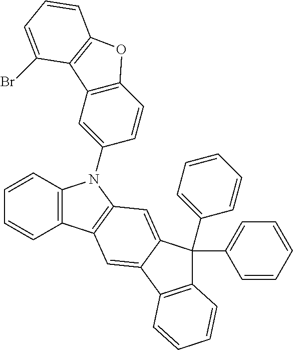 S161 |
| 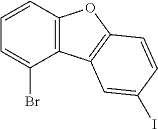 [SMILES: Brc1cccc2oc3ccc(I)cc3c12] CAS-1822311-11-4 | 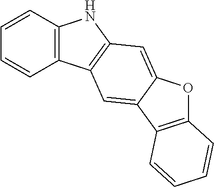 [SMILES: c1ccc2c(c1)[nH]c1cc3oc4ccccc4c3cc12] CAS-1246308-83-7 | 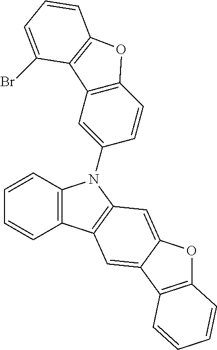 S162 |
| 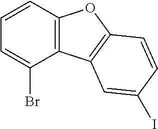 [SMILES: Brc1cccc2oc3ccc(I)cc3c12] CAS-1822311-11-4 | 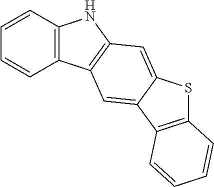 [SMILES: c1ccc2c(c1)[nH]c1cc3sc4ccccc4c3cc12] CAS-1255309-04-6 | 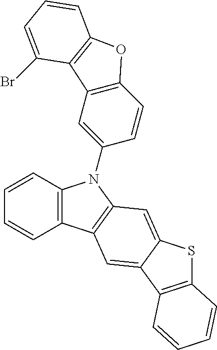 S163 |
| 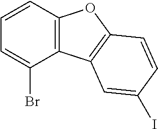 [SMILES: Brc1cccc2oc3ccc(I)cc3c12] CAS-1822311-11-4 | 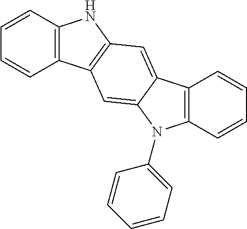 [SMILES: c1ccc(-n2c3ccccc3c3cc4[nH]c5ccccc5c4cc32)cc1] CAS-1316311-27-9 | 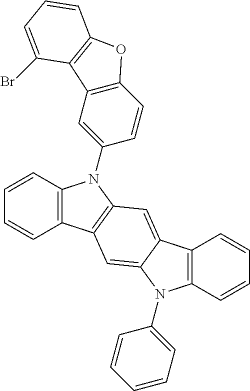 S164 |
| 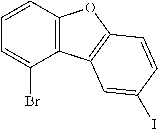 [SMILES: Brc1cccc2oc3ccc(I)cc3c12] CAS-1822311-11-4 | 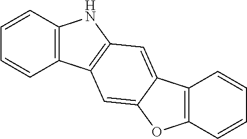 [SMILES: c1ccc2c(c1)[nH]c1cc3c(cc12)oc1ccccc13] CAS-1199350-22-5 | 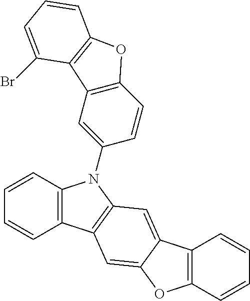 S165 |
| 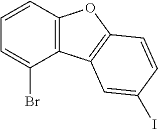 [SMILES: Brc1cccc2oc3ccc(I)cc3c12] CAS-1822311-11-4 | 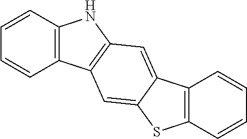 [SMILES: c1ccc2c(c1)[nH]c1cc3c(cc12)sc1ccccc13] CAS-1255309-10-4 | 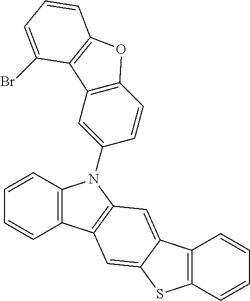 S166 |
| 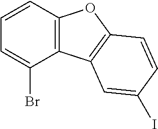 [SMILES: Brc1cccc2oc3ccc(I)cc3c12] CAS-1822311-11-4 | 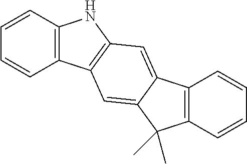 [SMILES: CC1(C)c2ccccc2-c2cc3[nH]c4ccccc4c3cc21] CAS-1260228-95-2 | 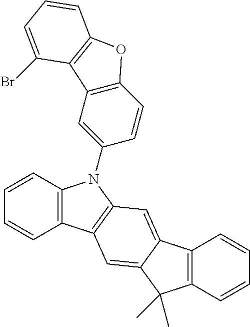 S167 |
| 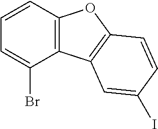 [SMILES: Brc1cccc2oc3ccc(I)cc3c12] CAS-1822311-11-4 | 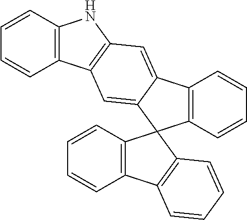 [SMILES: c1ccc2c(c1)-c1ccccc1C21c2ccccc2-c2cc3[nH]c4ccccc4c3cc21] CAS-1219841-59-4 | 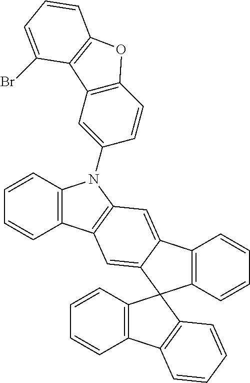 S168 |
| 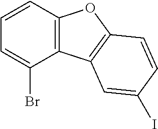 [SMILES: Brc1cccc2oc3ccc(I)cc3c12] CAS-1822311-11-4 | 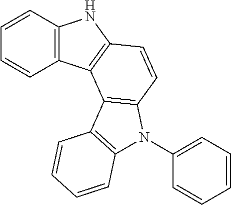 [SMILES: c1ccc(-n2c3ccccc3c3c4c(ccc32)[nH]c2ccccc24)cc1] CAS-1637752-63-6 | 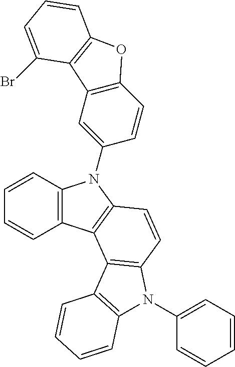 S169 |
| 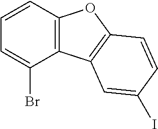 [SMILES: Brc1cccc2oc3ccc(I)cc3c12] CAS-1822311-11-4 | 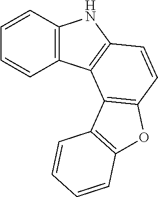 [SMILES: c1ccc2c(c1)[nH]c1ccc3oc4ccccc4c3c12] CAS-1622290-43-0 | 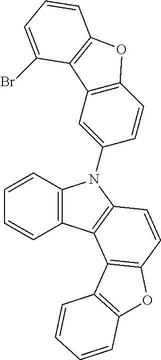 S170 |
| 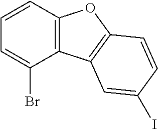 [SMILES: Brc1cccc2oc3ccc(I)cc3c12] CAS-1822311-11-4 | 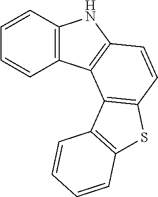 [SMILES: c1ccc2c(c1)[nH]c1ccc3sc4ccccc4c3c12] CAS-1255309-17-1 | 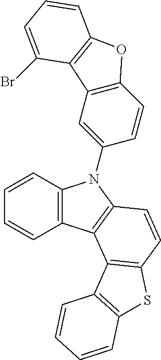 S171 |
| 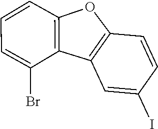 [SMILES: Brc1cccc2oc3ccc(I)cc3c12] CAS-1822311-11-4 | 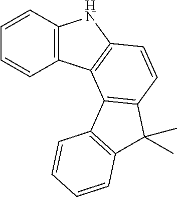 [SMILES: CC1(C)c2ccccc2-c2c1ccc1[nH]c3ccccc3c21] CAS-1623813-70-6 | 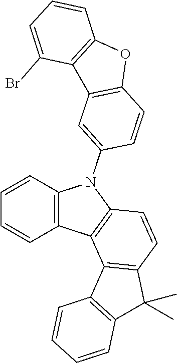 S172 |
| 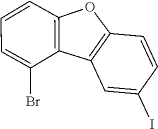 [SMILES: Brc1cccc2oc3ccc(I)cc3c12] CAS-1822311-11-4 | 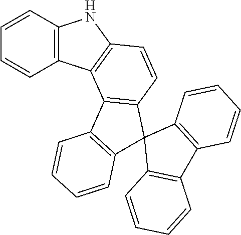 [SMILES: c1ccc2c(c1)-c1ccccc1C21c2ccccc2-c2c1ccc1[nH]c3ccccc3c21] CAS-1936530-01-6 | 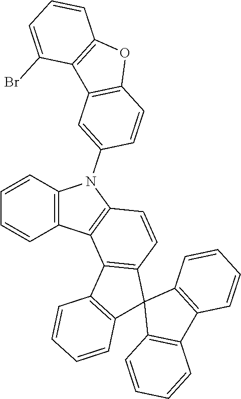 S173 |
| 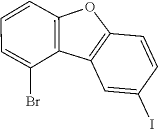 [SMILES: Brc1cccc2oc3ccc(I)cc3c12] CAS-1822311-11-4 | 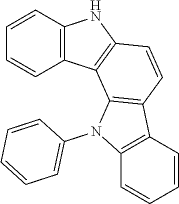 [SMILES: c1ccc(-n2c3ccccc3c3ccc4[nH]c5ccccc5c4c32)cc1] CAS-1346571-68-3 | 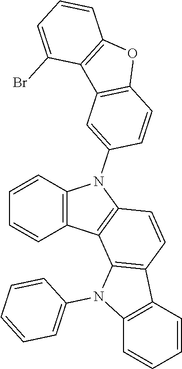 S174 |
| 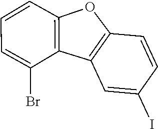 [SMILES: Brc1cccc2oc3ccc(I)cc3c12] CAS-1822311-11-4 | 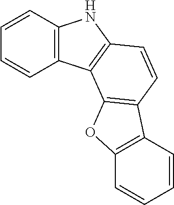 [SMILES: c1ccc2c(c1)[nH]c1ccc3c4ccccc4oc3c12] CAS-1199616-66-4 | 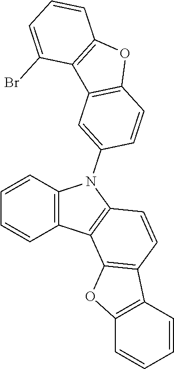 S175 |
| 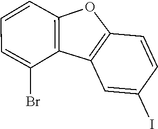 [SMILES: Brc1cccc2oc3ccc(I)cc3c12] CAS-1822311-11-4 | 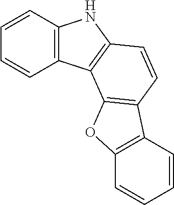 [SMILES: c1ccc2c(c1)[nH]c1ccc3c4ccccc4oc3c12] CAS-1255308-97-4 | 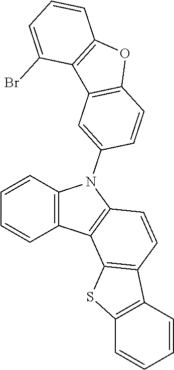 S176 |
| 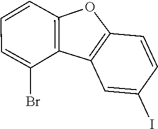 [SMILES: Brc1cccc2oc3ccc(I)cc3c12] CAS-1822311-11-4 | 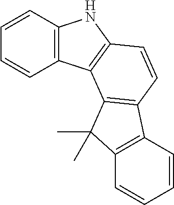 [SMILES: CC1(C)c2ccccc2-c2ccc3[nH]c4ccccc4c3c21] CAS-1346645-54-2 | 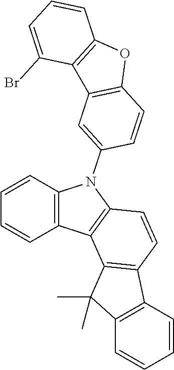 S177 |
| 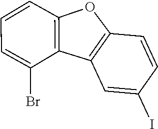 [SMILES: Brc1cccc2oc3ccc(I)cc3c12] CAS-1822311-11-4 | 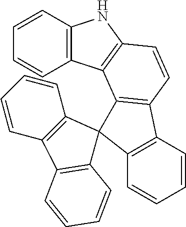 [SMILES: c1ccc2c(c1)-c1ccccc1C21c2ccccc2-c2ccc3[nH]c4ccccc4c3c21] CAS-2055578-08-8 | 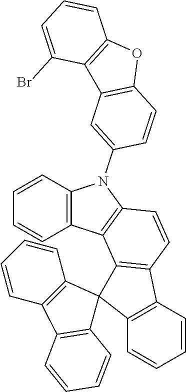 S178 |
| 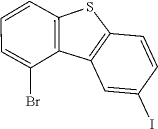 [SMILES: Brc1cccc2sc3ccc(I)cc3c12] CAS-1822311-12-5 | 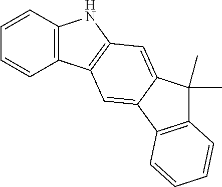 [SMILES: CC1(C)c2ccccc2-c2cc3c(cc21)[nH]c1ccccc13] CAS-1257220-47-5 | 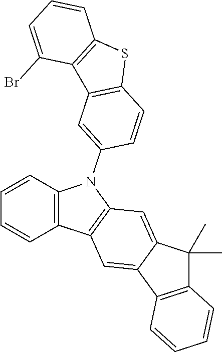 S179 |
| 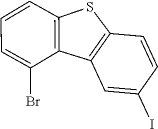 [SMILES: Brc1cccc2sc3ccc(I)cc3c12] CAS-1822311-12-5 | 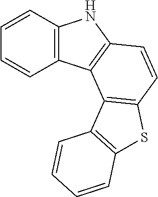 [SMILES: c1ccc2c(c1)[nH]c1ccc3sc4ccccc4c3c12] CAS-1255309-17-1 | 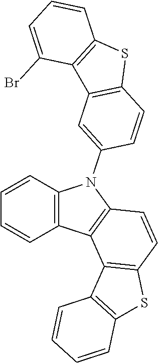 S180 |
| 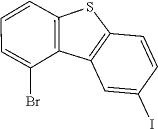 [SMILES: Brc1cccc2sc3ccc(I)cc3c12] CAS-1822311-12-5 | 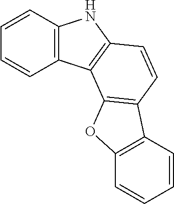 [SMILES: c1ccc2c(c1)[nH]c1ccc3c4ccccc4oc3c12] CAS-1199616-66-4 | 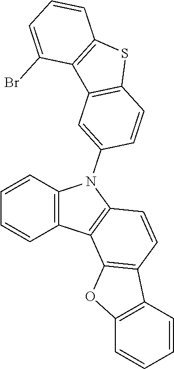 S181 |
| 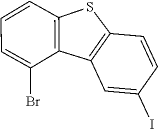 [SMILES: Brc1cccc2sc3ccc(I)cc3c12] CAS-1822311-12-5 | 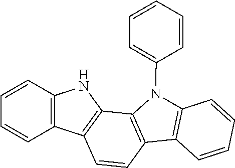 [SMILES: c1ccc(-n2c3ccccc3c3ccc4c5ccccc5[nH]c4c32)cc1] CAS-1024598-06-8 | 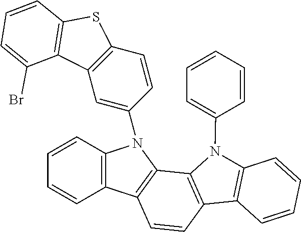 S182 |
| 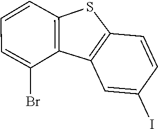 [SMILES: Brc1cccc2sc3ccc(I)cc3c12] CAS-1822311-12-5 | 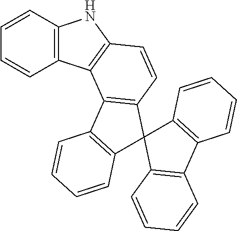 [SMILES: c1ccc2c(c1)-c1ccccc1C21c2ccccc2-c2c1ccc1[nH]c3ccccc3c21] CAS-1936530-01-6 | 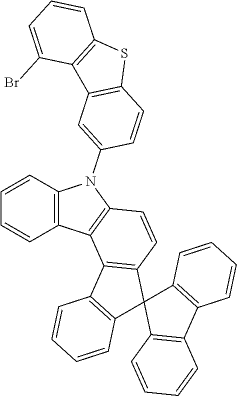 S183 |
| 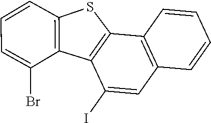 [SMILES: Brc1cccc2sc3c4ccccc4cc(I)c3c12] CAS-1883821-22-4 | 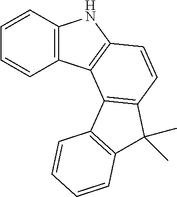 [SMILES: CC1(C)c2ccccc2-c2c1ccc1[nH]c3ccccc3c21] CAS-1623813-70-6 | 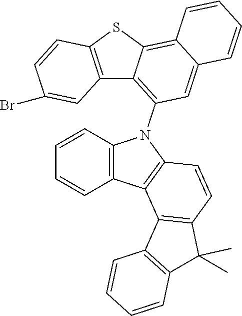 S184 |
| 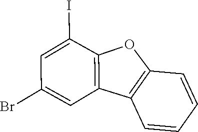 [SMILES: Brc1cc(I)c2oc3ccccc3c2c1] CAS-1401068-25-4 | 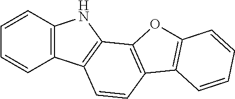 [SMILES: c1ccc2c(c1)[nH]c1c2ccc2c3ccccc3oc21] CAS-1338919-70-2 | 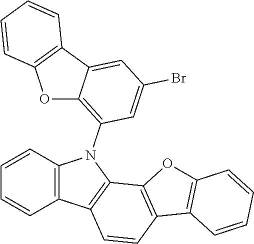 S185 |
| 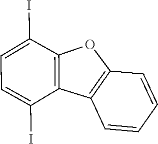 [SMILES: Ic1ccc(I)c2c1oc1ccccc12] CAS-1923736-35-9 | 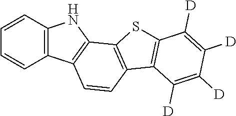 [SMILES: [2H]c1c([2H])c([2H])c2c(sc3c2ccc2c4ccccc4[nH]c23)c1[2H]] CAS-1447708-61-3 | 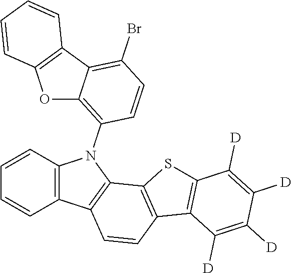 S186 |
| 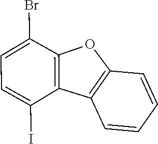 [SMILES: Brc1ccc(I)c2c1oc1ccccc12] CAS-2096506-51-1 | 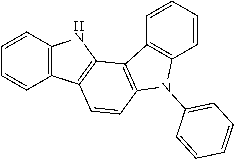 [SMILES: c1ccc(-n2c3ccccc3c3c4[nH]c5ccccc5c4ccc32)cc1] CAS-1247053-55-9 | 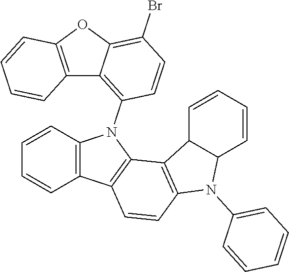 S187 |
| 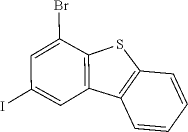 [SMILES: Brc1cc(I)cc2c1sc1ccccc12] CAS-1913276-15-9 | 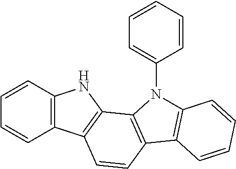 [SMILES: c1ccc(-n2c3ccccc3c3ccc4c5ccccc5[nH]c4c32)cc1] CAS-1024598-06-8 | 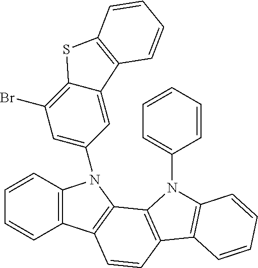 S188 |
| 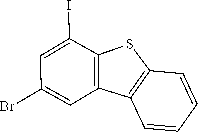 [SMILES: Brc1cc(I)c2sc3ccccc3c2c1] CAS-1923736-34-8 | 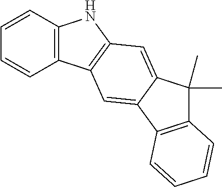 [SMILES: CC1(C)c2ccccc2-c2cc3c(cc21)[nH]c1ccccc13] CAS-1257220-47-5 | 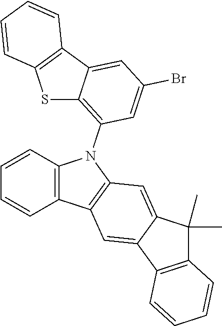 S189 |
| 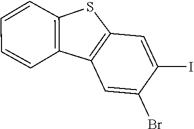 [SMILES: Brc1cc2c(cc1I)sc1ccccc12] CAS-1549979-42-1 | 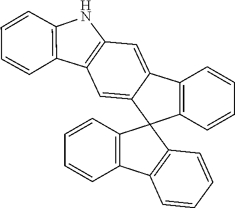 [SMILES: c1ccc2c(c1)-c1ccccc1C21c2ccccc2-c2cc3[nH]c4ccccc4c3cc21] CAS-1219841-59-4 | 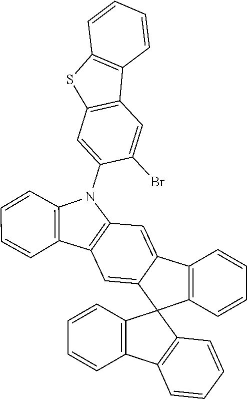 S190 |
| 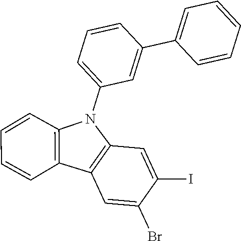 [SMILES: Brc1cc2c3ccccc3n(-c3cccc(-c4ccccc4)c3)c2cc1I] CAS-2086719-53-9 | 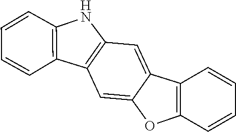 [SMILES: c1ccc2c(c1)[nH]c1cc3c(cc12)oc1ccccc13] CAS-1199350-22-5 | 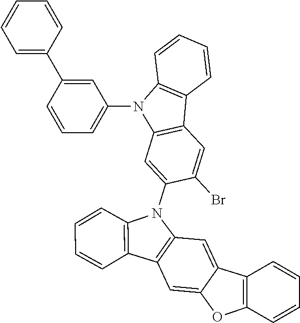 S191 |
| 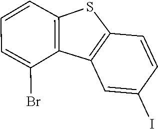 [SMILES: Brc1cccc2sc3ccc(I)cc3c12] CAS-1822311-12-5 | 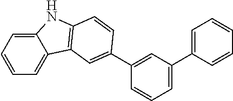 [SMILES: c1ccc(-c2cccc(-c3ccc4[nH]c5ccccc5c4c3)c2)cc1] CAS-1643526-99-1 | 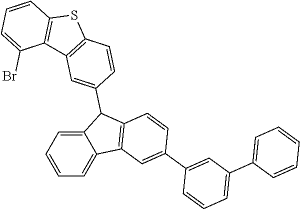 S192 |
| 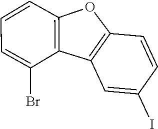 [SMILES: Brc1cccc2oc3ccc(I)cc3c12] CAS-1822311-11-4 | 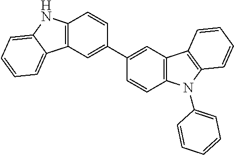 [SMILES: c1ccc(-n2c3ccccc3c3cc(-c4ccc5[nH]c6ccccc6c5c4)ccc32)cc1] CAS-1060735-14-9 | 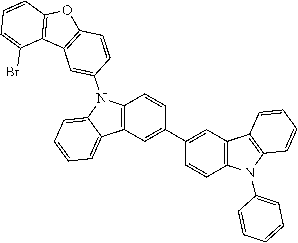 S193 |
| 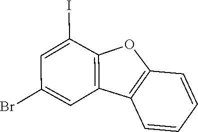 [SMILES: Brc1cc(I)c2oc3ccccc3c2c1] CAS-1401068-25-4 | 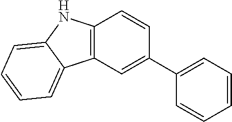 [SMILES: c1ccc(-c2ccc3[nH]c4ccccc4c3c2)cc1] CAS-103012-26-6 | 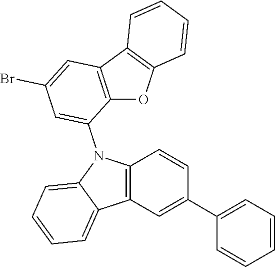 S194 |
| 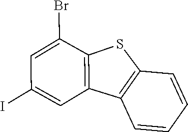 [SMILES: Brc1cc(I)cc2c1sc1ccccc12] CAS-1913276-15-9 | 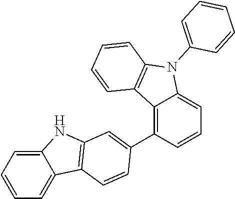 [SMILES: c1ccc(-n2c3ccccc3c3c(-c4ccc5c(c4)[nH]c4ccccc45)cccc32)cc1] CAS-2160600-08-6 | 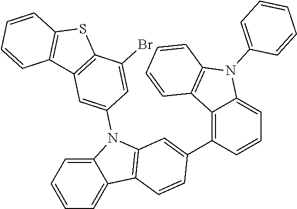 S195 |
| 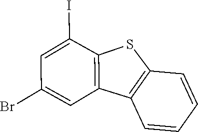 [SMILES: Brc1cc(I)c2sc3ccccc3c2c1] CAS-1923736-34-8 | 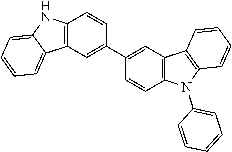 [SMILES: c1ccc(-n2c3ccccc3c3cc(-c4ccc5[nH]c6ccccc6c5c4)ccc32)cc1] CAS-1060735-14-9 | 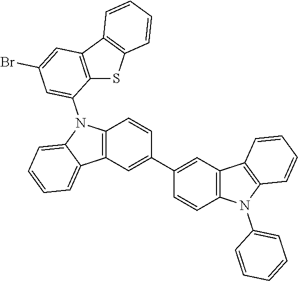 S196 |

### Preparation of the Products:

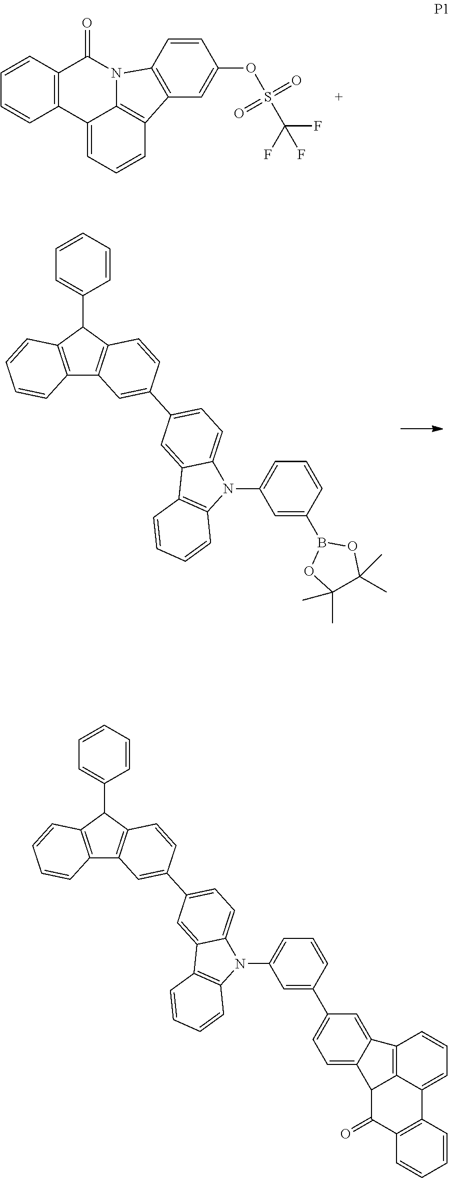 [SMILES: O=c1c2ccccc2c2cccc3c4cc(OS(=O)(=O)C(F)(F)F)ccc4n1c23 ; CC1(C)OB(c2cccc(-n3c4ccccc4c4cc(-c5ccc6c(c5)-c5ccccc5C6c5ccccc5)ccc43)c2)OC1(C)C ; O=C1c2ccccc2-c2cccc3c2C1c1ccc(-c2cccc(-n4c5ccccc5c5cc(-c6ccc7c(c6)-c6ccccc6C7c6ccccc6)ccc54)c2)cc1-3]

To an initial charge, in a flask, of S100 (15.02 g, 36.0 mmol), 9-phenyl-9′-[3-(4,4,5,5-tetramethyl-[1,3,2]dioxaborolan-2-yl)-phenyl]-9H,9′H-[3,3′]bicarbazolyl [2088364-11-6] (26.41 g, 43.3 mmol) and tripotassium phosphate (15.54 g) are added tetrahydrofuran (400 ml) and water (100 ml), and the mixture is inertized with argon for 30 min. Subsequently, palladium(II) acetate [3375-31-3] (204.3 mg) and X-Phos [564483-18-7] (905 mg) are added and the mixture is heated under reflux for 20 h. After cooling, the precipitated solids are filtered off with suction and washed twice with water and twice with THF. The crude product is subjected to hot extraction four times with toluene and finally sublimed under high vacuum. Yield: 15.6 g (20.7 mmol, 58%) of yellow solid, purity: >99.9% by HPLC.

In an analogous manner, it is possible to prepare the following compounds: The phosphine ligand used here may also be S-Phos [657408-07-6] rather than X-Phos, or the catalyst system (palladium source and ligand) used may be bis(triphenylphosphine)palladium chloride [13965-03-2]. Purification can also be effected using column chromatography, or recrystallization or hot extraction using other standard solvents such as ethanol, butanol, acetone, ethyl acetate, acetonitrile, toluene, xylene, dichloromethane, methanol, tetrahydrofuran, n-butyl acetate, 1,4-dioxane, or recrystallization using high boilers such as dimethyl sulfoxide, N,N-dimethylformamide, N,N-dimethylacetamide, N-methylpyrrolidone, etc. The yields are typically in the range between 15% and 75%.

| Reactant | | |
|---|---|---|
| 1 | Reactant 2 | Product |
| S100 | 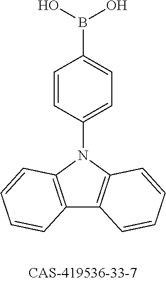 [SMILES: OB(O)c1ccc(-n2c3ccccc3c3ccccc32)cc1] | 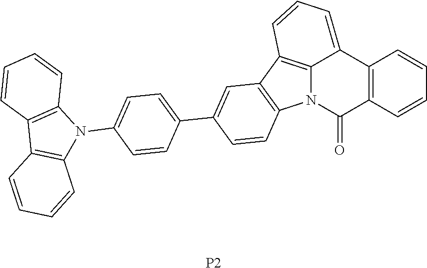 [SMILES: O=c1c2ccccc2c2cccc3c4cc(-c5ccc(-n6c7ccccc7c7ccccc76)cc5)ccc4n1c23 ; "P2"] |
| S100 | 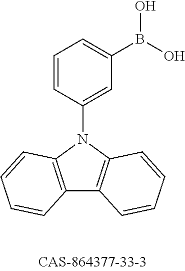 [SMILES: OB(O)c1cccc(-n2c3ccccc3c3ccccc32)c1] | 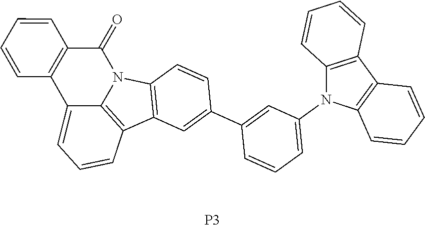 [SMILES: O=c1c2ccccc2c2cccc3c4cc(-c5cccc(-n6c7ccccc7c7ccccc76)c5)ccc4n1c23 ; "P3"] |
| S100 | 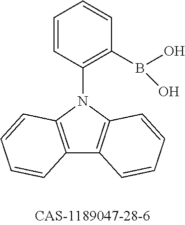 [SMILES: OB(O)c1ccccc1-n1c2ccccc2c2ccccc21] | 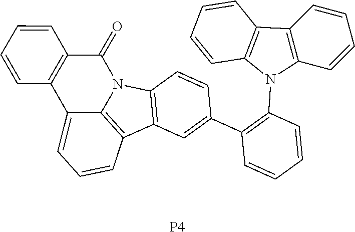 |
| S100 | 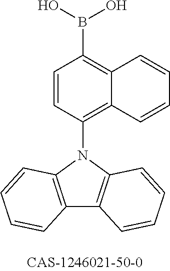 [SMILES: OB(O)c1ccc(-n2c3ccccc3c3ccccc32)c2ccccc12] | 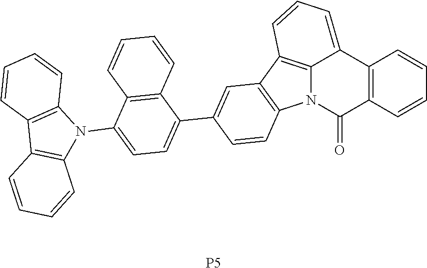 [SMILES: O=c1c2ccccc2c2cccc3c4cc(-c5ccc(-n6c7ccccc7c7ccccc76)c6ccccc56)ccc4n1c23 ; "P5"] |
| S100 | 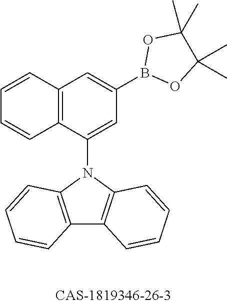 [SMILES: CC1(C)OB(c2cc(-n3c4ccccc4c4ccccc43)c3ccccc3c2)OC1(C)C] | 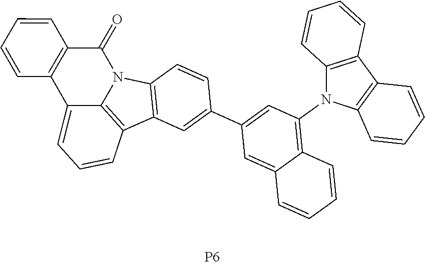 [SMILES: O=c1c2ccccc2c2cccc3c4cc(-c5cc(-n6c7ccccc7c7ccccc76)c6ccccc6c5)ccc4n1c23 ; "P6"] |
| S100 | 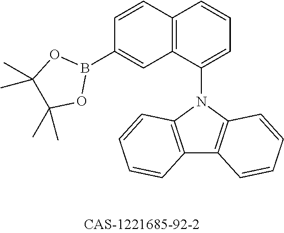 [SMILES: CC1(C)OB(c2ccc3cccc(-n4c5ccccc5c5ccccc54)c3c2)OC1(C)C] | 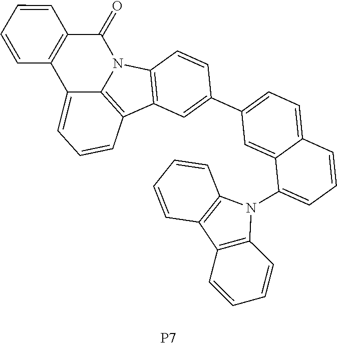 [SMILES: O=c1c2ccccc2c2cccc3c4cc(-c5ccc6cccc(-n7c8ccccc8c8ccccc87)c6c5)ccc4n1c23 ; "P7"] |
| S100 | 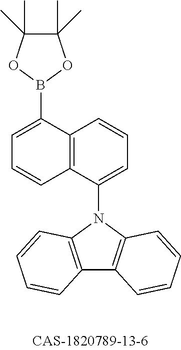 [SMILES: CC1(C)OB(c2cccc3c(-n4c5ccccc5c5ccccc54)cccc23)OC1(C)C] | 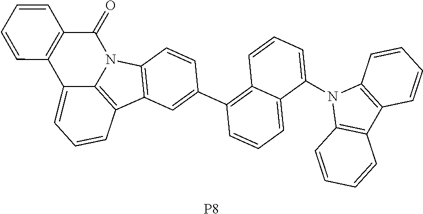 [SMILES: O=c1c2ccccc2c2cccc3c4cc(-c5cccc6c(-n7c8ccccc8c8ccccc87)cccc56)ccc4n1c23 ; "P8"] |
| S100 | 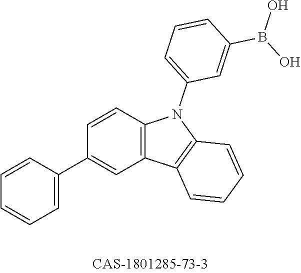 [SMILES: OB(O)c1cccc(-n2c3ccccc3c3cc(-c4ccccc4)ccc32)c1] | 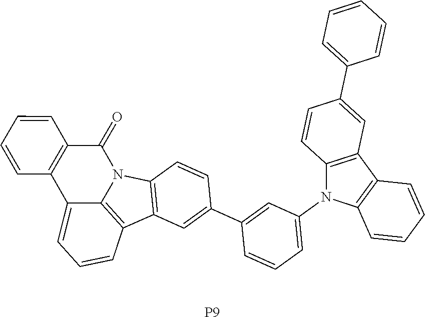 |
| S100 | 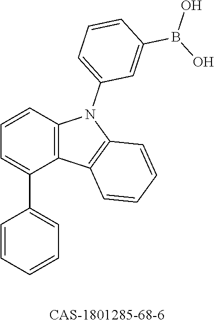 [SMILES: OB(O)c1cccc(-n2c3ccccc3c3c(-c4ccccc4)cccc32)c1] | 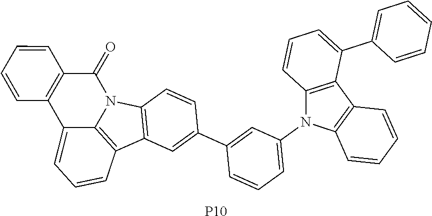 |
| S100 | 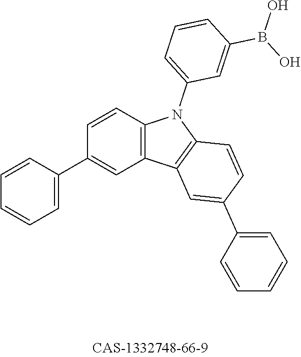 [SMILES: OB(O)c1cccc(-n2c3ccc(-c4ccccc4)cc3c3cc(-c4ccccc4)ccc32)c1] | 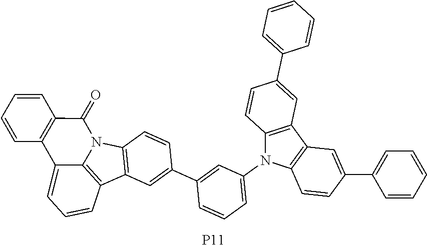 |
| S100 | 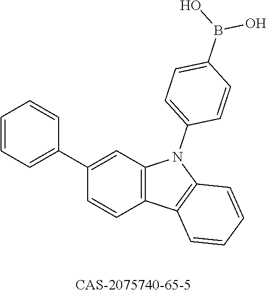 [SMILES: OB(O)c1ccc(-n2c3ccccc3c3ccc(-c4ccccc4)cc32)cc1] | 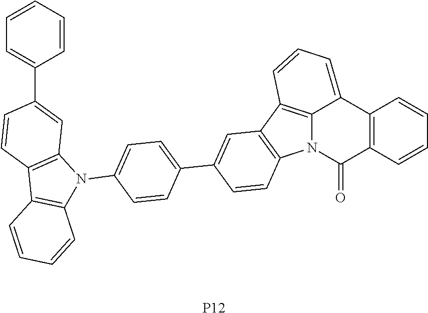 [SMILES: O=c1c2ccccc2c2cccc3c4cc(-c5ccc(-n6c7ccccc7c7ccc(-c8ccccc8)cc76)cc5)ccc4n1c23 ; "P12"] |
| S100 | 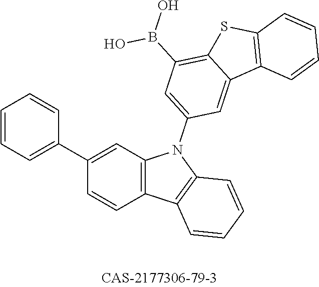 [SMILES: OB(O)c1cc(-n2c3ccccc3c3ccc(-c4ccccc4)cc32)cc2c1sc1ccccc12] | 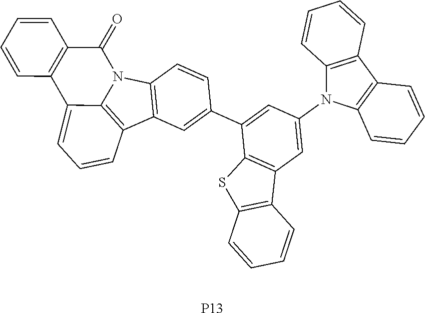 [SMILES: O=c1c2ccccc2c2cccc3c4cc(-c5cc(-n6c7ccccc7c7ccccc76)cc6c5sc5ccccc56)ccc4n1c23 ; "P13"] |
| S100 | 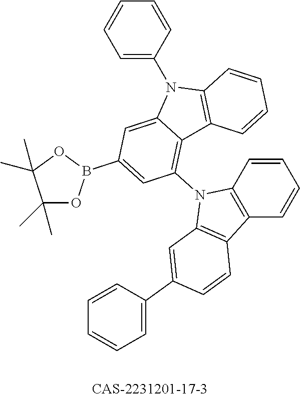 [SMILES: CC1(C)OB(c2cc(-n3c4ccccc4c4ccc(-c5ccccc5)cc43)c3c4ccccc4n(-c4ccccc4)c3c2)OC1(C)C] | 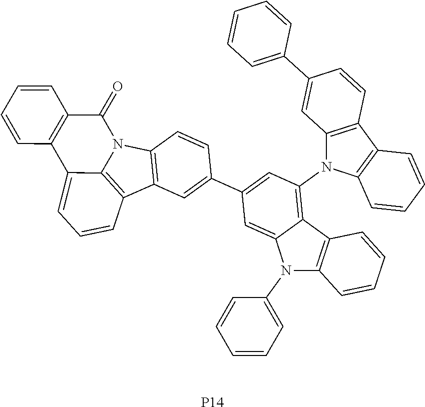 [SMILES: O=c1c2ccccc2c2cccc3c4cc(-c5cc(-n6c7ccccc7c7ccc(-c8ccccc8)cc76)c6c7ccccc7n(-c7ccccc7)c6c5)ccc4n1c23 ; "P14"] |
| S100 | 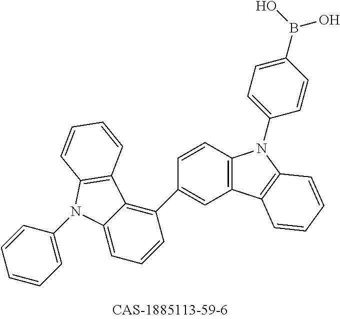 [SMILES: OB(O)c1ccc(-n2c3ccccc3c3cc(-c4cccc5c4c4ccccc4n5-c4ccccc4)ccc32)cc1] | 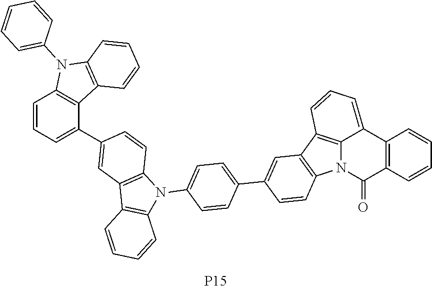 |
| S100 | 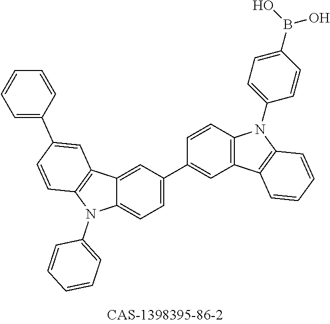 [SMILES: OB(O)c1ccc(-n2c3ccccc3c3cc(-c4ccc5c(c4)c4cc(-c6ccccc6)ccc4n5-c4ccccc4)ccc32)cc1] | 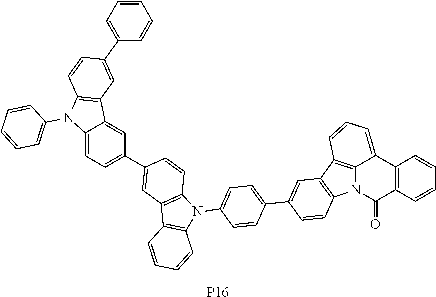 |
| S100 | 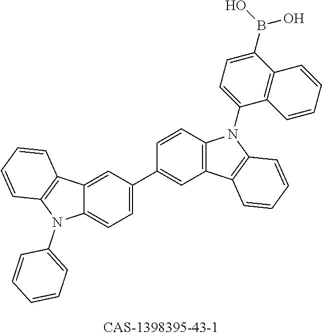 [SMILES: OB(O)c1ccc(-n2c3ccccc3c3cc(-c4ccc5c(c4)c4ccccc4n5-c4ccccc4)ccc32)c2ccccc12] | 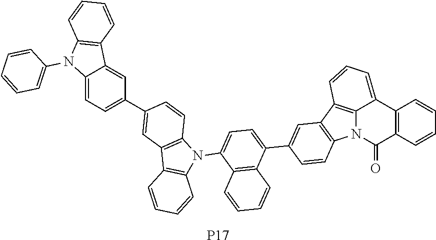 |
| S100 | 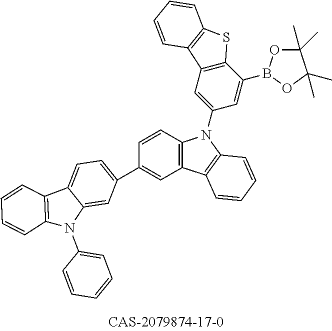 [SMILES: CC1(C)OB(c2cc(-n3c4ccccc4c4cc(-c5ccc6c7ccccc7n(-c7ccccc7)c6c5)ccc43)cc3c2sc2ccccc23)OC1(C)C] | 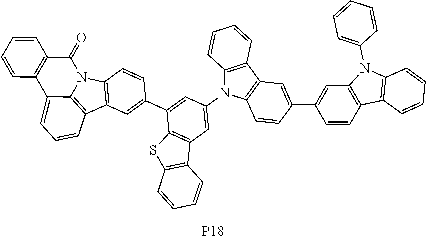 [SMILES: O=c1c2ccccc2c2cccc3c4cc(-c5cc(-n6c7ccccc7c7cc(-c8ccc9c%10ccccc%10n(-c%10ccccc%10)c9c8)ccc76)cc6c5sc5ccccc56)ccc4n1c23 ; "P18"] |
| S100 | 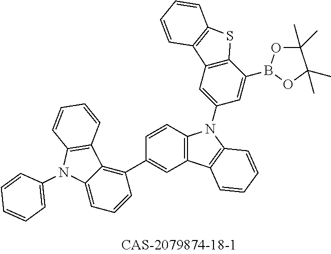 [SMILES: CC1(C)OB(c2cc(-n3c4ccccc4c4cc(-c5cccc6c5c5ccccc5n6-c5ccccc5)ccc43)cc3c2sc2ccccc23)OC1(C)C] | 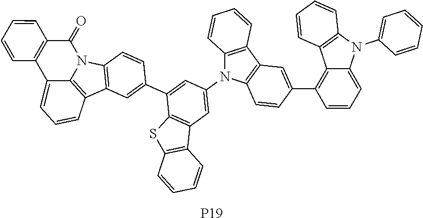 [SMILES: O=c1c2ccccc2c2cccc3c4cc(-c5cc(-n6c7ccccc7c7cc(-c8cccc9c8c8ccccc8n9-c8ccccc8)ccc76)cc6c5sc5ccccc56)ccc4n1c23 ; "P19"] |
| S100 | 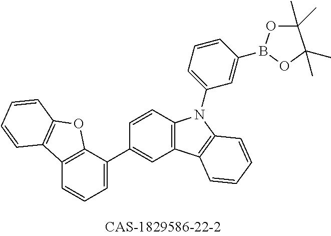 [SMILES: CC1(C)OB(c2cccc(-n3c4ccccc4c4cc(-c5cccc6c5oc5ccccc56)ccc43)c2)OC1(C)C] | 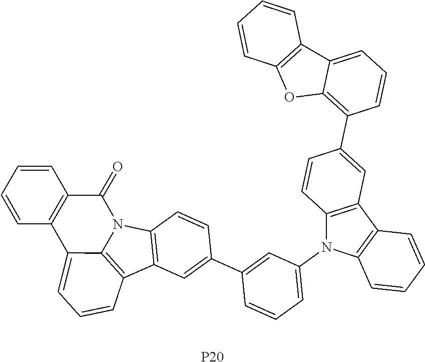 |
| S100 | 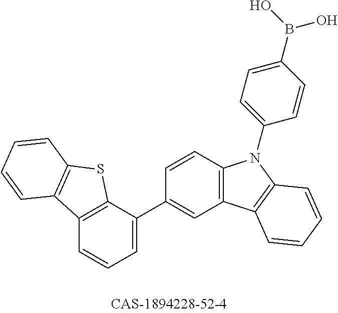 [SMILES: OB(O)c1ccc(-n2c3ccccc3c3cc(-c4cccc5c4sc4ccccc45)ccc32)cc1] | 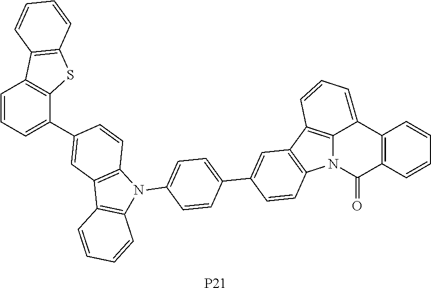 |
| S101 | 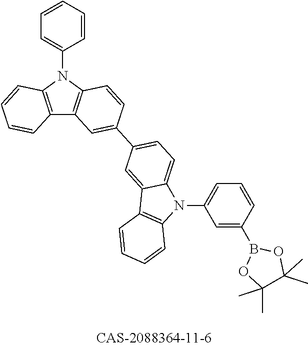 [SMILES: CC1(C)OB(c2cccc(-n3c4ccccc4c4cc(-c5ccc6c(c5)c5ccccc5n6-c5ccccc5)ccc43)c2)OC1(C)C] | 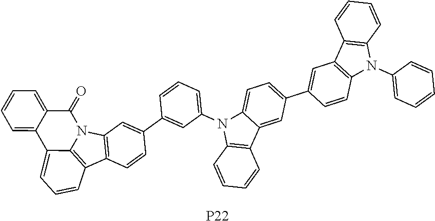 |
| S102 | 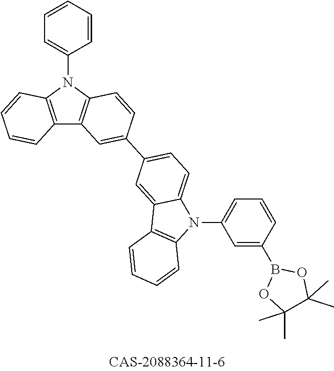 [SMILES: CC1(C)OB(c2cccc(-n3c4ccccc4c4cc(-c5ccc6c(c5)c5ccccc5n6-c5ccccc5)ccc43)c2)OC1(C)C] | 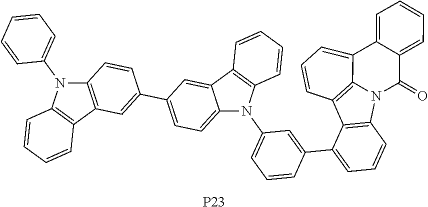 |
| S103 | 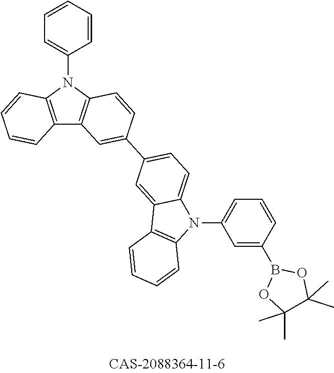 [SMILES: CC1(C)OB(c2cccc(-n3c4ccccc4c4cc(-c5ccc6c(c5)c5ccccc5n6-c5ccccc5)ccc43)c2)OC1(C)C] | 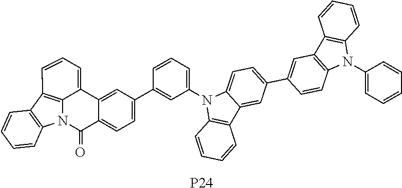 |
| S104 | 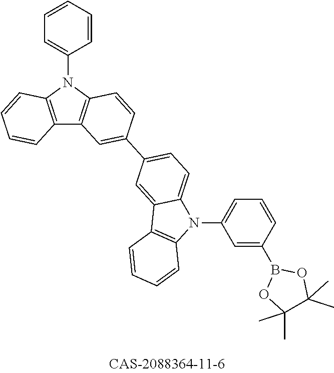 [SMILES: CC1(C)OB(c2cccc(-n3c4ccccc4c4cc(-c5ccc6c(c5)c5ccccc5n6-c5ccccc5)ccc43)c2)OC1(C)C] | 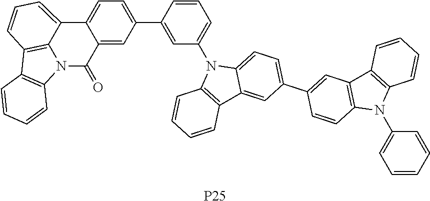 |
| S105 | 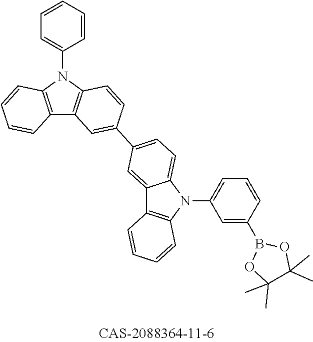 [SMILES: CC1(C)OB(c2cccc(-n3c4ccccc4c4cc(-c5ccc6c(c5)c5ccccc5n6-c5ccccc5)ccc43)c2)OC1(C)C] | 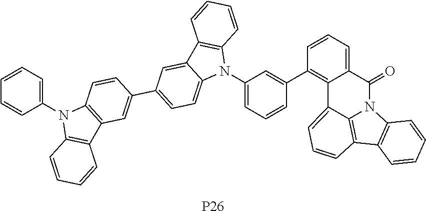 |
| S106 | 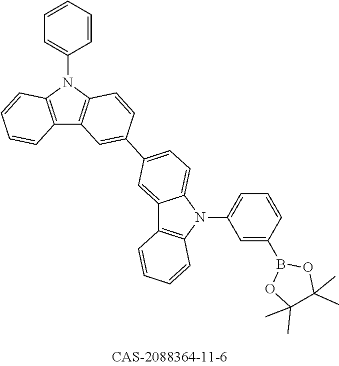 [SMILES: CC1(C)OB(c2cccc(-n3c4ccccc4c4cc(-c5ccc6c(c5)c5ccccc5n6-c5ccccc5)ccc43)c2)OC1(C)C] | 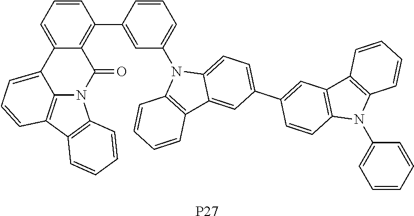 |
| S107 | 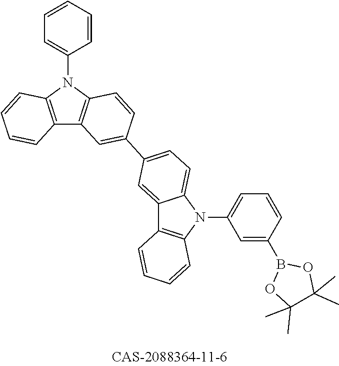 [SMILES: CC1(C)OB(c2cccc(-n3c4ccccc4c4cc(-c5ccc6c(c5)c5ccccc5n6-c5ccccc5)ccc43)c2)OC1(C)C] | 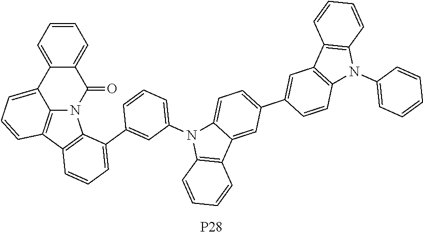 |
| S108 | 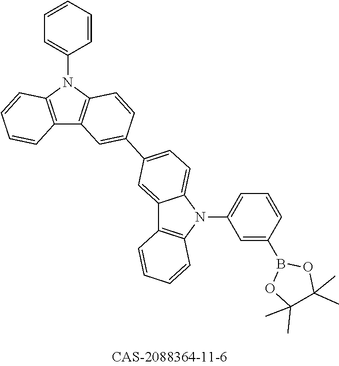 [SMILES: CC1(C)OB(c2cccc(-n3c4ccccc4c4cc(-c5ccc6c(c5)c5ccccc5n6-c5ccccc5)ccc43)c2)OC1(C)C] | 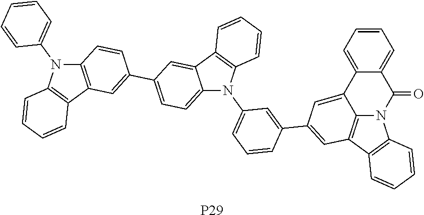 |
| S109 | 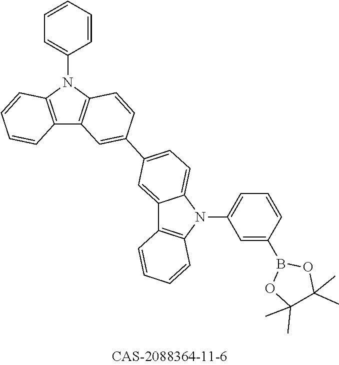 [SMILES: CC1(C)OB(c2cccc(-n3c4ccccc4c4cc(-c5ccc6c(c5)c5ccccc5n6-c5ccccc5)ccc43)c2)OC1(C)C] | 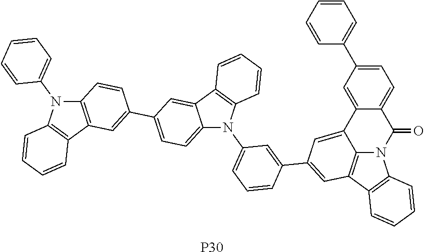 |
| S110 | 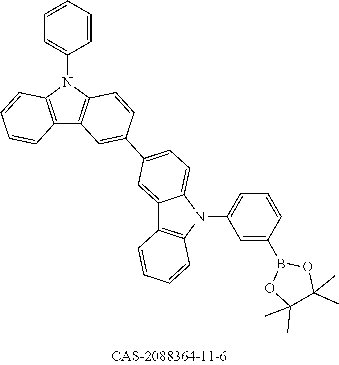 [SMILES: CC1(C)OB(c2cccc(-n3c4ccccc4c4cc(-c5ccc6c(c5)c5ccccc5n6-c5ccccc5)ccc43)c2)OC1(C)C] | 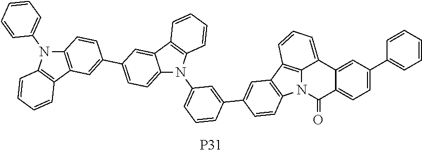 |
| S111 | 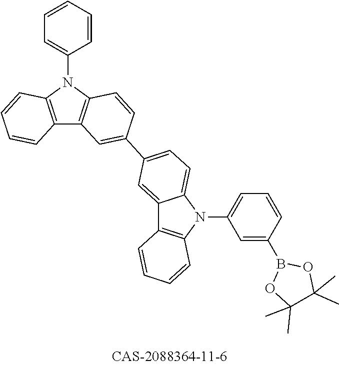 [SMILES: CC1(C)OB(c2cccc(-n3c4ccccc4c4cc(-c5ccc6c(c5)c5ccccc5n6-c5ccccc5)ccc43)c2)OC1(C)C] | 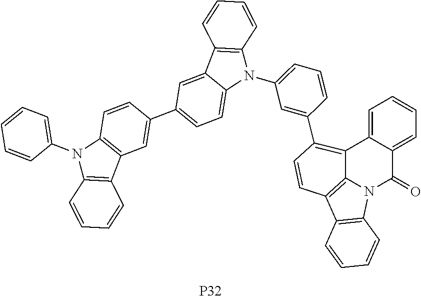 |
| S112 | 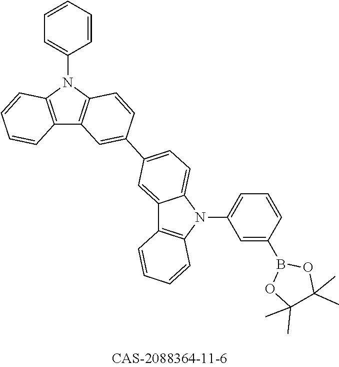 [SMILES: CC1(C)OB(c2cccc(-n3c4ccccc4c4cc(-c5ccc6c(c5)c5ccccc5n6-c5ccccc5)ccc43)c2)OC1(C)C] | 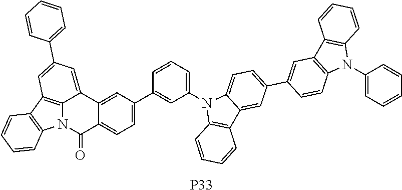 |
| S113 | 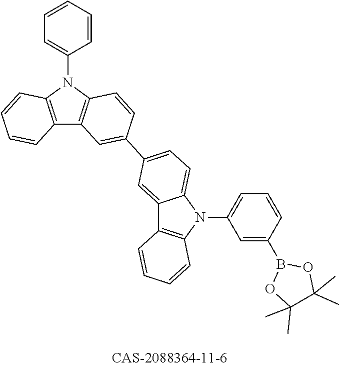 [SMILES: CC1(C)OB(c2cccc(-n3c4ccccc4c4cc(-c5ccc6c(c5)c5ccccc5n6-c5ccccc5)ccc43)c2)OC1(C)C] | 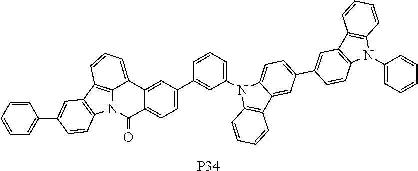 |
| S114 | 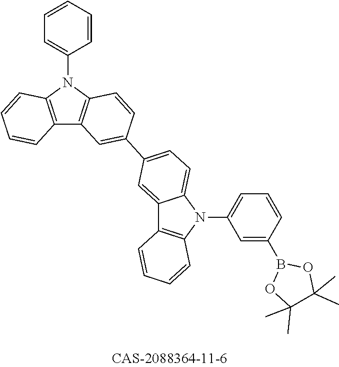 [SMILES: CC1(C)OB(c2cccc(-n3c4ccccc4c4cc(-c5ccc6c(c5)c5ccccc5n6-c5ccccc5)ccc43)c2)OC1(C)C] | 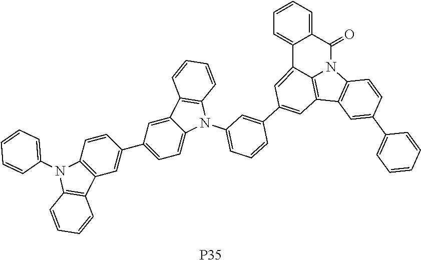 |
| S115 | 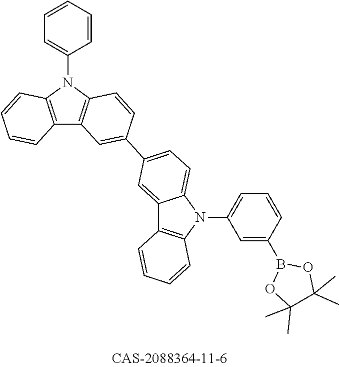 [SMILES: CC1(C)OB(c2cccc(-n3c4ccccc4c4cc(-c5ccc6c(c5)c5ccccc5n6-c5ccccc5)ccc43)c2)OC1(C)C] | 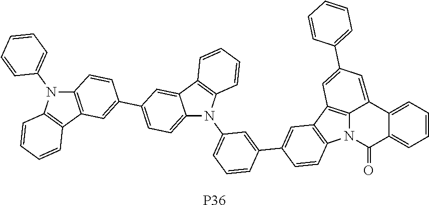 |
| S101 | 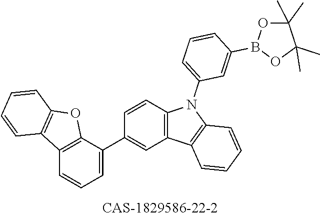 [SMILES: CC1(C)OB(c2cccc(-n3c4ccccc4c4cc(-c5cccc6c5oc5ccccc56)ccc43)c2)OC1(C)C] | 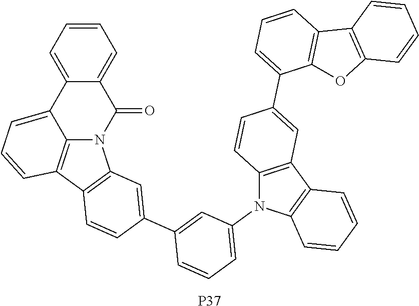 |
| S102 | 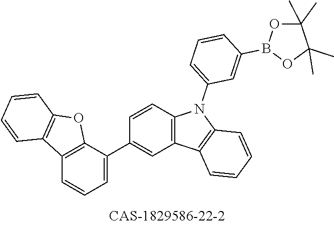 [SMILES: CC1(C)OB(c2cccc(-n3c4ccccc4c4cc(-c5cccc6c5oc5ccccc56)ccc43)c2)OC1(C)C] | 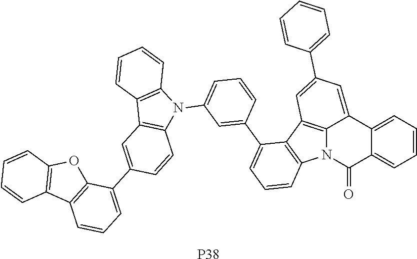 |
| S103 | 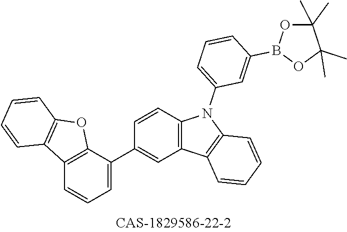 [SMILES: CC1(C)OB(c2cccc(-n3c4ccccc4c4cc(-c5cccc6c5oc5ccccc56)ccc43)c2)OC1(C)C] | 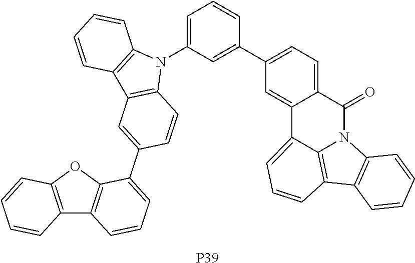 |
| S104 | 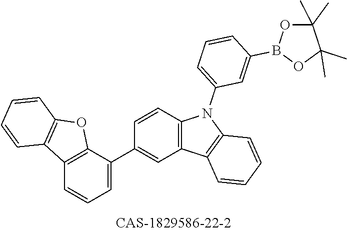 [SMILES: CC1(C)OB(c2cccc(-n3c4ccccc4c4cc(-c5cccc6c5oc5ccccc56)ccc43)c2)OC1(C)C] | 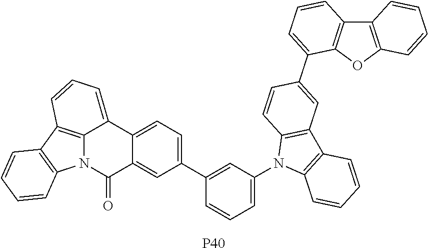 |
| S105 | 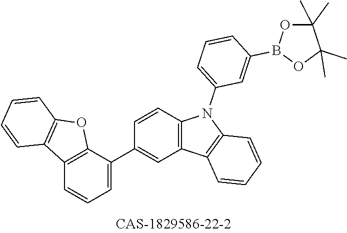 [SMILES: CC1(C)OB(c2cccc(-n3c4ccccc4c4cc(-c5cccc6c5oc5ccccc56)ccc43)c2)OC1(C)C] | 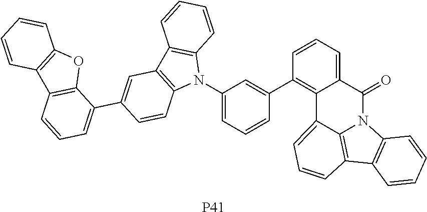 |
| S106 | 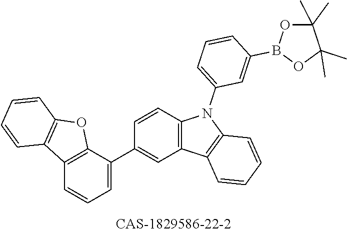 [SMILES: CC1(C)OB(c2cccc(-n3c4ccccc4c4cc(-c5cccc6c5oc5ccccc56)ccc43)c2)OC1(C)C] | 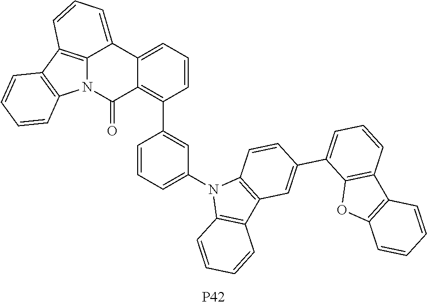 [SMILES: O=c1c2c(-c3cccc(-n4c5ccccc5c5cc(-c6cccc7c6oc6ccccc67)ccc54)c3)cccc2c2cccc3c4ccccc4n1c23 ; "P42"] |
| S107 | 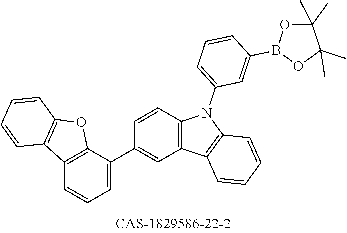 [SMILES: CC1(C)OB(c2cccc(-n3c4ccccc4c4cc(-c5cccc6c5oc5ccccc56)ccc43)c2)OC1(C)C] | 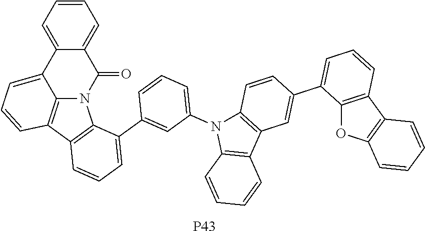 |
| S108 | 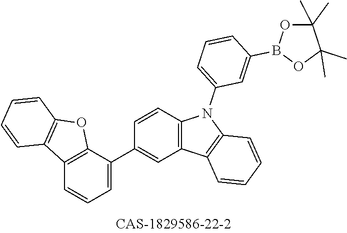 [SMILES: CC1(C)OB(c2cccc(-n3c4ccccc4c4cc(-c5cccc6c5oc5ccccc56)ccc43)c2)OC1(C)C] | 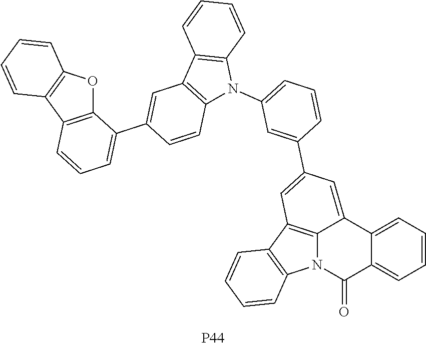 |
| S109 | 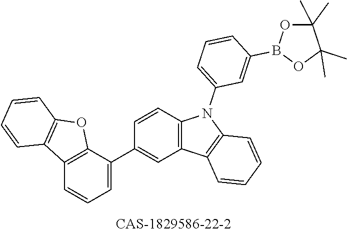 [SMILES: CC1(C)OB(c2cccc(-n3c4ccccc4c4cc(-c5cccc6c5oc5ccccc56)ccc43)c2)OC1(C)C] | 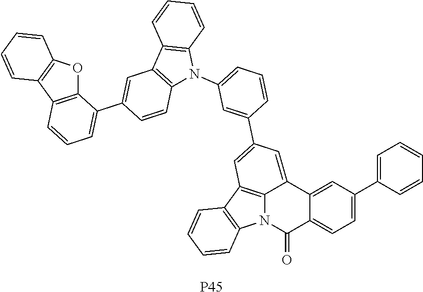 |
| S110 | 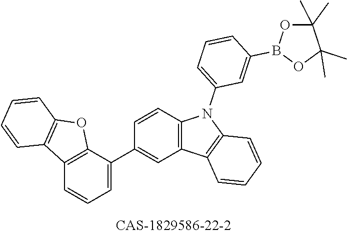 [SMILES: CC1(C)OB(c2cccc(-n3c4ccccc4c4cc(-c5cccc6c5oc5ccccc56)ccc43)c2)OC1(C)C] | 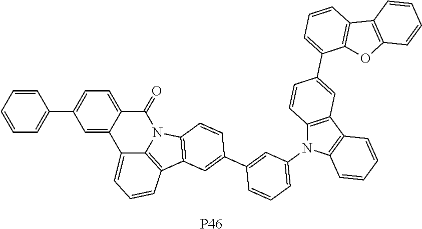 |
| S111 | 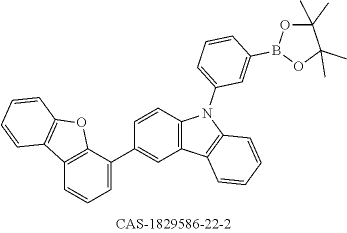 [SMILES: CC1(C)OB(c2cccc(-n3c4ccccc4c4cc(-c5cccc6c5oc5ccccc56)ccc43)c2)OC1(C)C] | 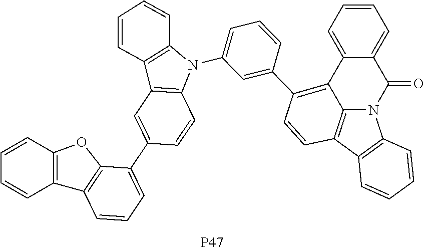 [SMILES: O=c1c2ccccc2c2c(-c3cccc(-n4c5ccccc5c5cc(-c6cccc7c6oc6ccccc67)ccc54)c3)ccc3c4ccccc4n1c32 ; "P47"] |
| S112 | 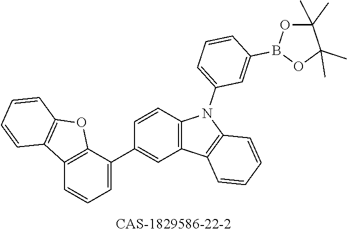 [SMILES: CC1(C)OB(c2cccc(-n3c4ccccc4c4cc(-c5cccc6c5oc5ccccc56)ccc43)c2)OC1(C)C] | 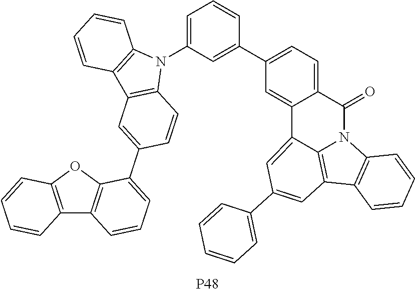 [SMILES: O=c1c2ccc(-c3cccc(-n4c5ccccc5c5cc(-c6cccc7c6oc6ccccc67)ccc54)c3)cc2c2cc(-c3ccccc3)cc3c4ccccc4n1c23 ; "P48"] |
| S113 | 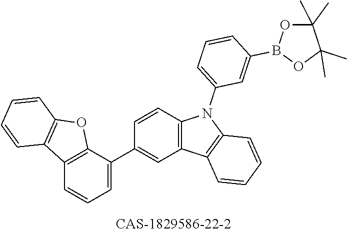 [SMILES: CC1(C)OB(c2cccc(-n3c4ccccc4c4cc(-c5cccc6c5oc5ccccc56)ccc43)c2)OC1(C)C] | 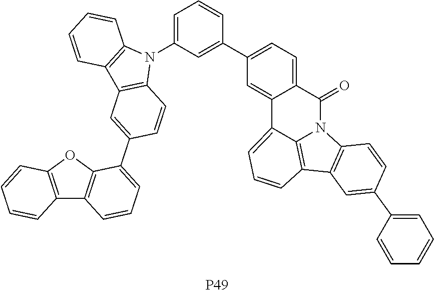 |
| S114 | 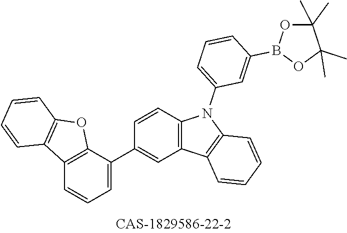 [SMILES: CC1(C)OB(c2cccc(-n3c4ccccc4c4cc(-c5cccc6c5oc5ccccc56)ccc43)c2)OC1(C)C] | 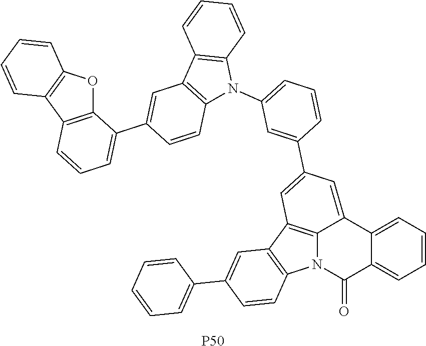 [SMILES: O=c1c2ccccc2c2cc(-c3cccc(-n4c5ccccc5c5cc(-c6cccc7c6oc6ccccc67)ccc54)c3)cc3c4cc(-c5ccccc5)ccc4n1c23 ; "P50"] |
| S115 | 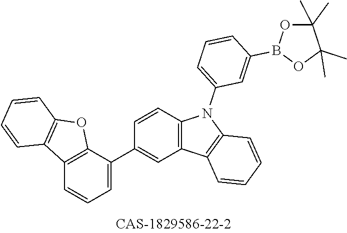 [SMILES: CC1(C)OB(c2cccc(-n3c4ccccc4c4cc(-c5cccc6c5oc5ccccc56)ccc43)c2)OC1(C)C] | 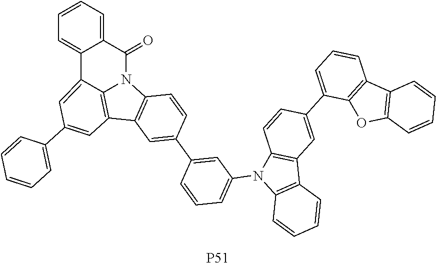 |
| S103 | 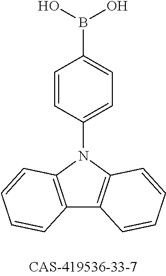 [SMILES: OB(O)c1ccc(-n2c3ccccc3c3ccccc32)cc1] | 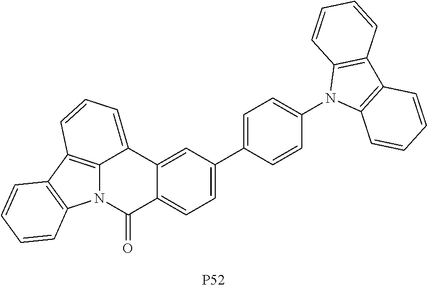 |
| S103 | 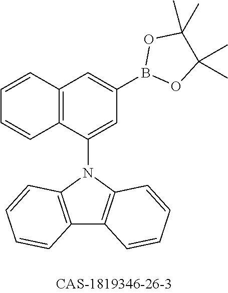 [SMILES: CC1(C)OB(c2cc(-n3c4ccccc4c4ccccc43)c3ccccc3c2)OC1(C)C] | 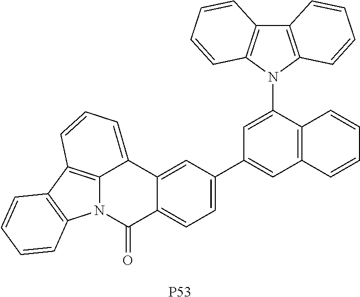 [SMILES: "P53" ; O=c1c2ccc(-c3cc(-n4c5ccccc5c5ccccc54)c4ccccc4c3)cc2c2cccc3c4ccccc4n1c23] |
| S103 | 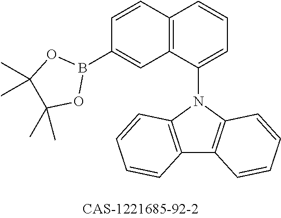 [SMILES: CC1(C)OB(c2ccc3cccc(-n4c5ccccc5c5ccccc54)c3c2)OC1(C)C] | 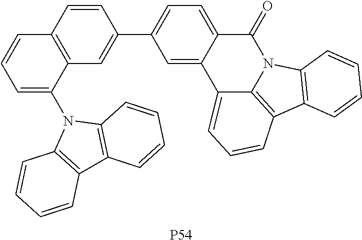 |
| S103 | 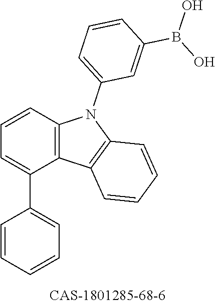 [SMILES: OB(O)c1cccc(-n2c3ccccc3c3c(-c4ccccc4)cccc32)c1] | 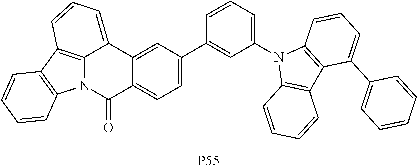 |
| S103 | 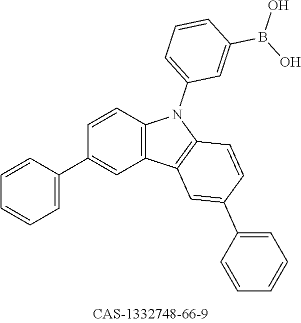 [SMILES: OB(O)c1cccc(-n2c3ccc(-c4ccccc4)cc3c3cc(-c4ccccc4)ccc32)c1] | 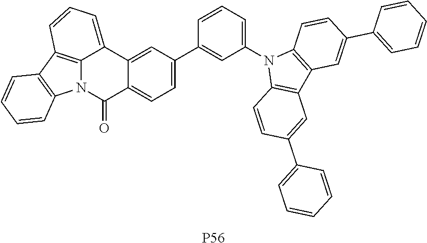 |
| S103 | 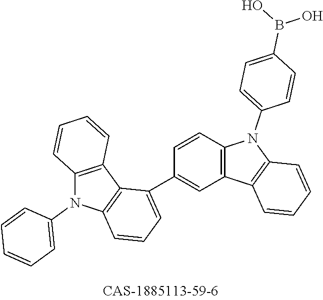 [SMILES: OB(O)c1ccc(-n2c3ccccc3c3cc(-c4cccc5c4c4ccccc4n5-c4ccccc4)ccc32)cc1] | 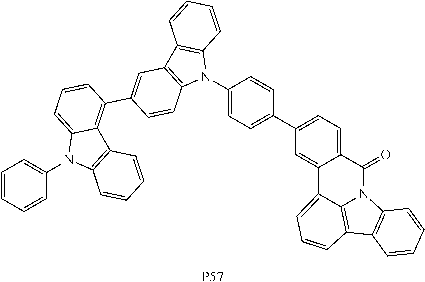 |
| S103 | 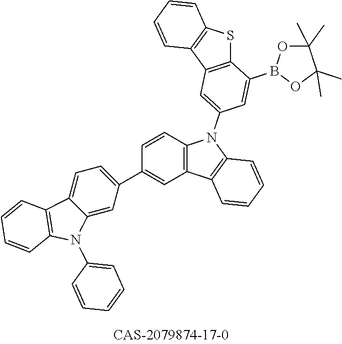 [SMILES: CC1(C)OB(c2cc(-n3c4ccccc4c4cc(-c5ccc6c7ccccc7n(-c7ccccc7)c6c5)ccc43)cc3c2sc2ccccc23)OC1(C)C] | 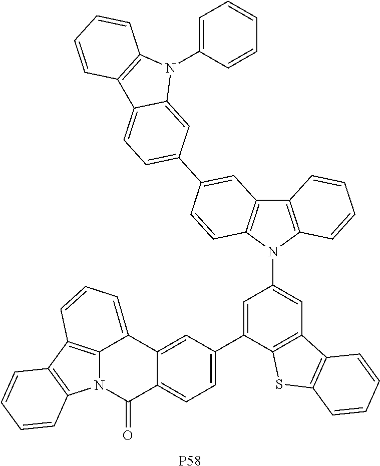 [SMILES: O=c1c2ccc(-c3cc(-n4c5ccccc5c5cc(-c6ccc7c8ccccc8n(-c8ccccc8)c7c6)ccc54)cc4c3sc3ccccc34)cc2c2cccc3c4ccccc4n1c23 ; "P58"] |
| S103 | 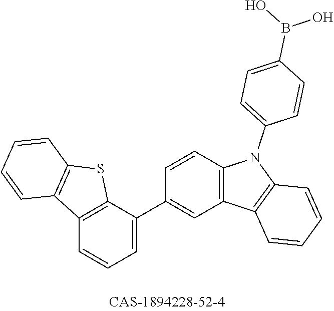 [SMILES: OB(O)c1ccc(-n2c3ccccc3c3cc(-c4cccc5c4sc4ccccc45)ccc32)cc1] | 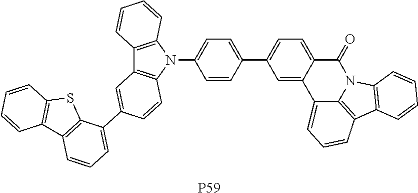 [SMILES: O=c1c2ccc(-c3ccc(-n4c5ccccc5c5cc(-c6cccc7c6sc6ccccc67)ccc54)cc3)cc2c2cccc3c4ccccc4n1c23 ; "P59"] |
| S108 | 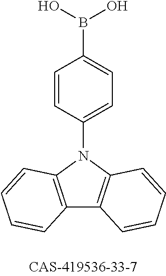 [SMILES: OB(O)c1ccc(-n2c3ccccc3c3ccccc32)cc1] | 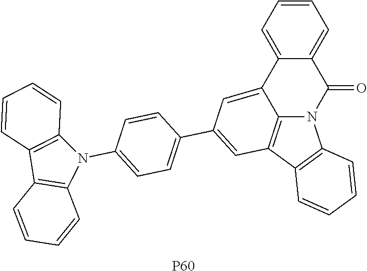 |
| S108 | 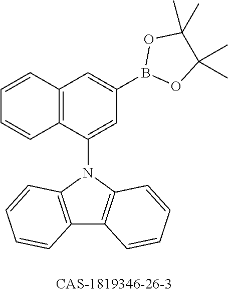 [SMILES: CC1(C)OB(c2cc(-n3c4ccccc4c4ccccc43)c3ccccc3c2)OC1(C)C] | 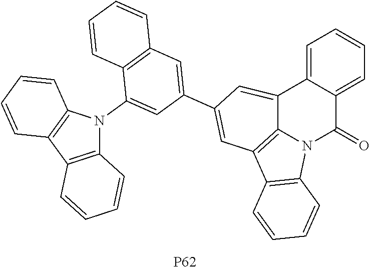 |
| S108 | 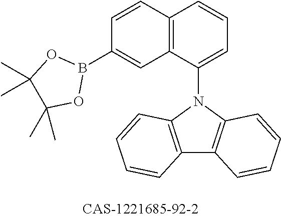 [SMILES: CC1(C)OB(c2ccc3cccc(-n4c5ccccc5c5ccccc54)c3c2)OC1(C)C] | 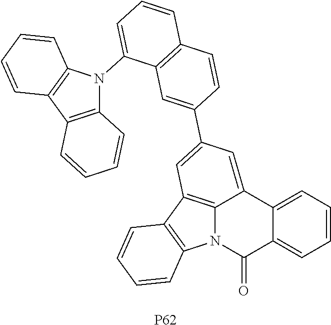 [SMILES: "P62" ; O=c1c2ccccc2c2cc(-c3ccc4cccc(-n5c6ccccc6c6ccccc65)c4c3)cc3c4ccccc4n1c23] |
| S108 | 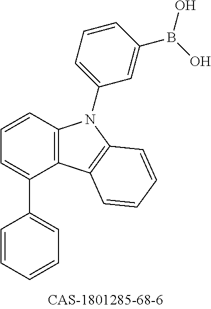 [SMILES: OB(O)c1cccc(-n2c3ccccc3c3c(-c4ccccc4)cccc32)c1] | 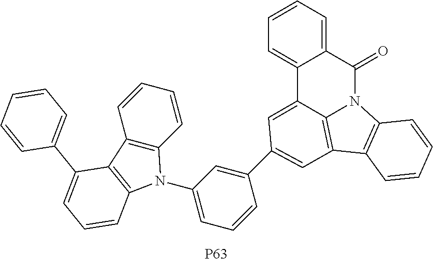 |
| S108 | 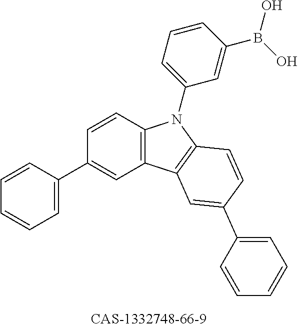 [SMILES: OB(O)c1cccc(-n2c3ccc(-c4ccccc4)cc3c3cc(-c4ccccc4)ccc32)c1] | 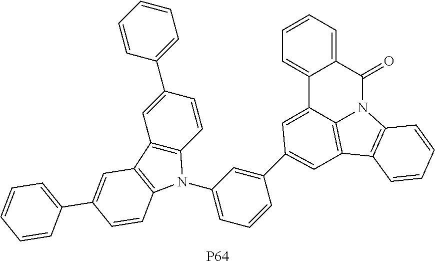 [SMILES: O=c1c2ccccc2c2cc(-c3cccc(-n4c5ccc(-c6ccccc6)cc5c5cc(-c6ccccc6)ccc54)c3)cc3c4ccccc4n1c23 ; "P64"] |
| S108 | 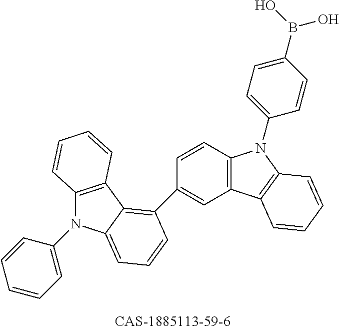 [SMILES: OB(O)c1ccc(-n2c3ccccc3c3cc(-c4cccc5c4c4ccccc4n5-c4ccccc4)ccc32)cc1] | 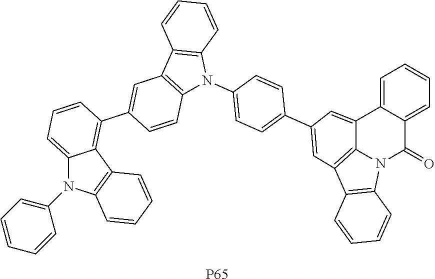 [SMILES: O=c1c2ccccc2c2cc(-c3ccc(-n4c5ccccc5c5cc(-c6cccc7c6c6ccccc6n7-c6ccccc6)ccc54)cc3)cc3c4ccccc4n1c23 ; "P65"] |
| S108 | 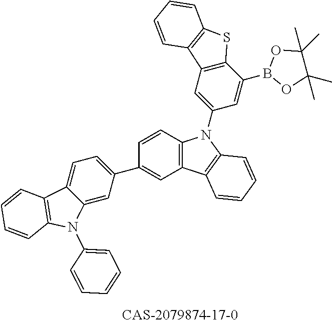 [SMILES: CC1(C)OB(c2cc(-n3c4ccccc4c4cc(-c5ccc6c7ccccc7n(-c7ccccc7)c6c5)ccc43)cc3c2sc2ccccc23)OC1(C)C] | 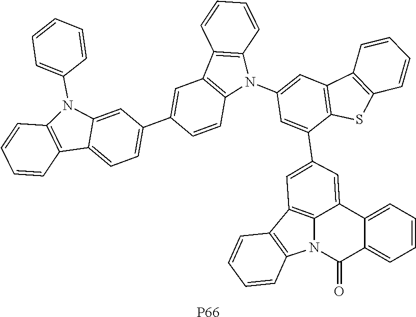 |
| S108 | 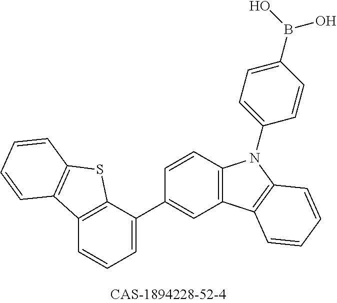 [SMILES: OB(O)c1ccc(-n2c3ccccc3c3cc(-c4cccc5c4sc4ccccc45)ccc32)cc1] | 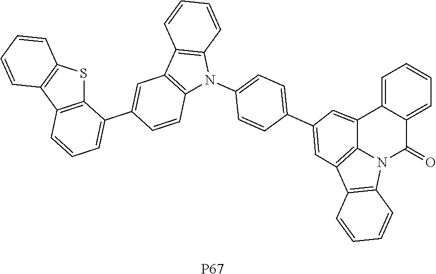 |
| S110 | 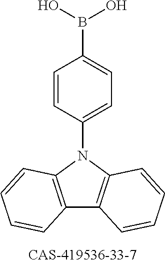 [SMILES: OB(O)c1ccc(-n2c3ccccc3c3ccccc32)cc1] | 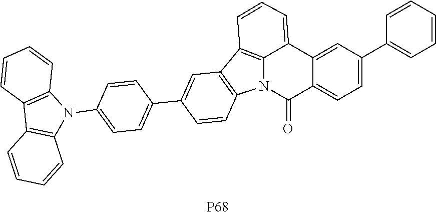 |
| S110 | 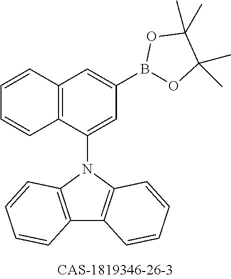 [SMILES: CC1(C)OB(c2cc(-n3c4ccccc4c4ccccc43)c3ccccc3c2)OC1(C)C] | 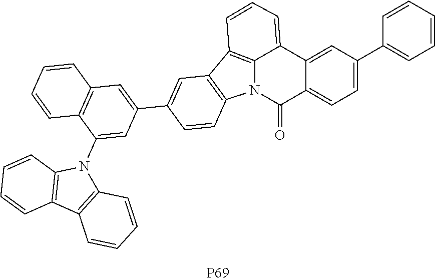 |
| S110 | 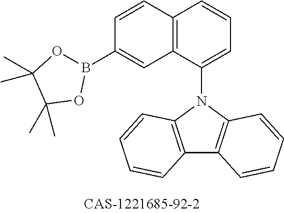 [SMILES: CC1(C)OB(c2ccc3cccc(-n4c5ccccc5c5ccccc54)c3c2)OC1(C)C] | 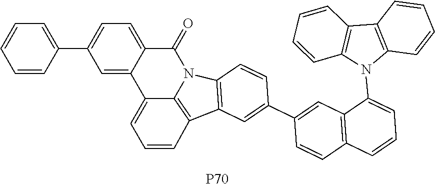 |
| S110 | 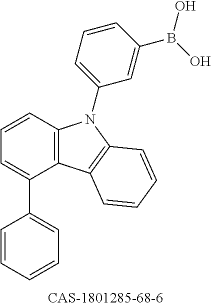 [SMILES: OB(O)c1cccc(-n2c3ccccc3c3c(-c4ccccc4)cccc32)c1] | 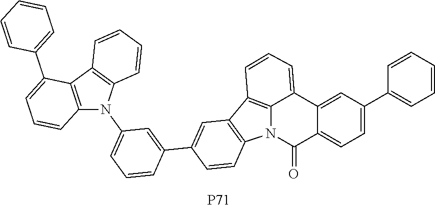 |
| S110 | 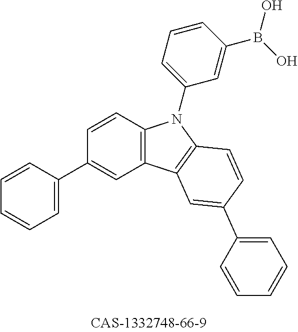 [SMILES: OB(O)c1cccc(-n2c3ccc(-c4ccccc4)cc3c3cc(-c4ccccc4)ccc32)c1] | 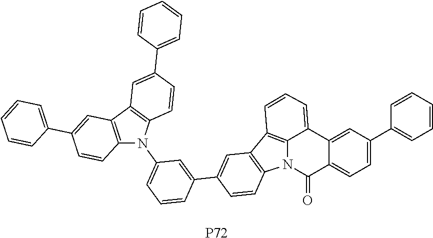 [SMILES: "P72" ; O=c1c2ccc(-c3ccccc3)cc2c2cccc3c4cc(-c5cccc(-n6c7ccc(-c8ccccc8)cc7c7cc(-c8ccccc8)ccc76)c5)ccc4n1c23] |
| S110 | 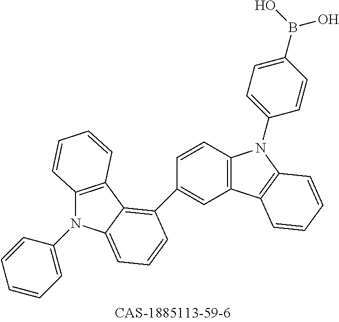 [SMILES: OB(O)c1ccc(-n2c3ccccc3c3cc(-c4cccc5c4c4ccccc4n5-c4ccccc4)ccc32)cc1] | 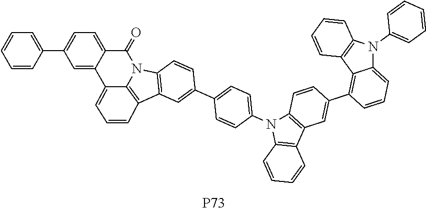 |
| S110 | 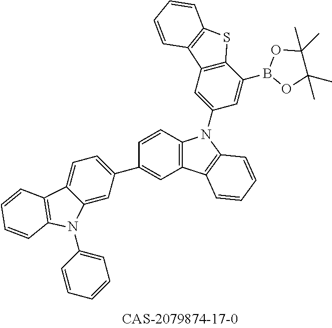 [SMILES: CC1(C)OB(c2cc(-n3c4ccccc4c4cc(-c5ccc6c7ccccc7n(-c7ccccc7)c6c5)ccc43)cc3c2sc2ccccc23)OC1(C)C] | 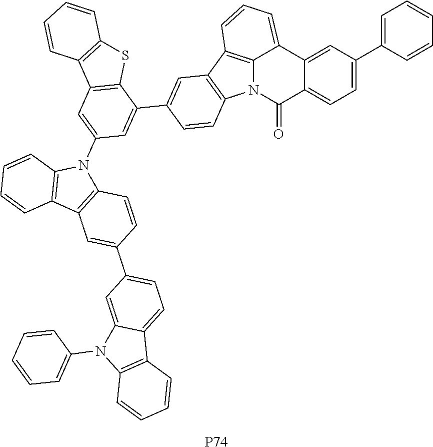 [SMILES: O=c1c2ccc(-c3ccccc3)cc2c2cccc3c4cc(-c5cc(-n6c7ccccc7c7cc(-c8ccc9c%10ccccc%10n(-c%10ccccc%10)c9c8)ccc76)cc6c5sc5ccccc56)ccc4n1c23 ; "P74"] |
| S110 | 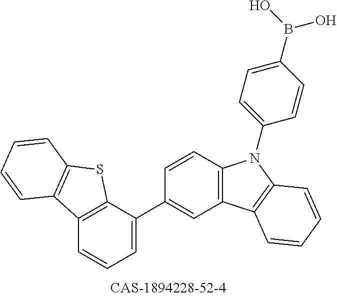 [SMILES: OB(O)c1ccc(-n2c3ccccc3c3cc(-c4cccc5c4sc4ccccc45)ccc32)cc1] | 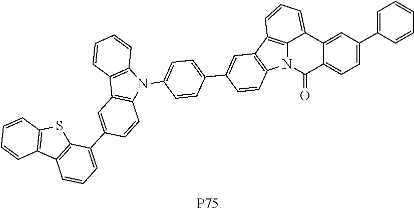 [SMILES: O=c1c2ccc(-c3ccccc3)cc2c2cccc3c4cc(-c5ccc(-n6c7ccccc7c7cc(-c8cccc9c8sc8ccccc89)ccc76)cc5)ccc4n1c23 ; "P75"] |
| S114 | 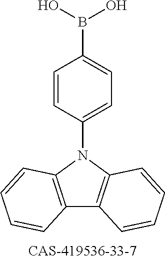 [SMILES: OB(O)c1ccc(-n2c3ccccc3c3ccccc32)cc1] | 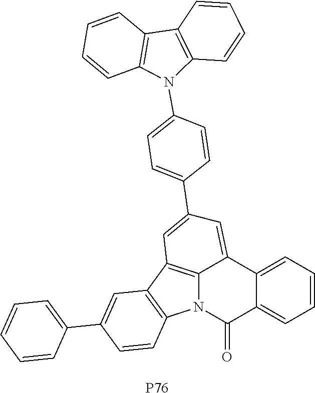 [SMILES: "P76" ; O=c1c2ccccc2c2cc(-c3ccc(-n4c5ccccc5c5ccccc54)cc3)cc3c4cc(-c5ccccc5)ccc4n1c23] |
| S114 | 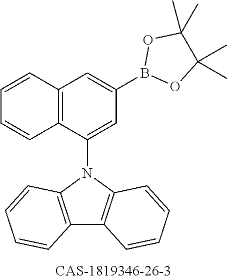 [SMILES: CC1(C)OB(c2cc(-n3c4ccccc4c4ccccc43)c3ccccc3c2)OC1(C)C] | 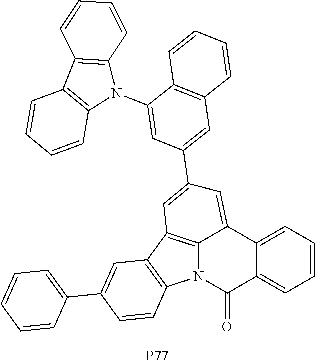 [SMILES: "P77" ; O=c1c2ccccc2c2cc(-c3cc(-n4c5ccccc5c5ccccc54)c4ccccc4c3)cc3c4cc(-c5ccccc5)ccc4n1c23] |
| S114 | 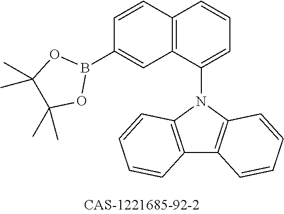 [SMILES: CC1(C)OB(c2ccc3cccc(-n4c5ccccc5c5ccccc54)c3c2)OC1(C)C] | 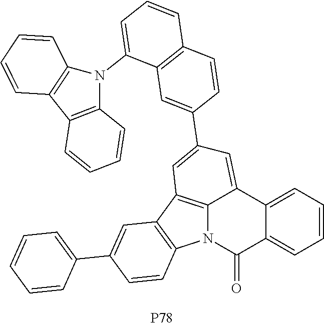 [SMILES: "P78" ; O=c1c2ccccc2c2cc(-c3ccc4cccc(-n5c6ccccc6c6ccccc65)c4c3)cc3c4cc(-c5ccccc5)ccc4n1c23] |
| S114 | 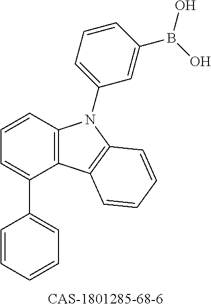 [SMILES: OB(O)c1cccc(-n2c3ccccc3c3c(-c4ccccc4)cccc32)c1] | 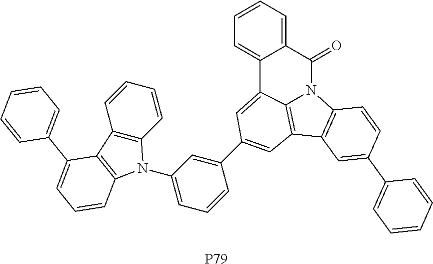 |
| S114 | 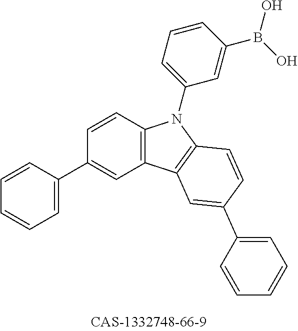 [SMILES: OB(O)c1cccc(-n2c3ccc(-c4ccccc4)cc3c3cc(-c4ccccc4)ccc32)c1] | 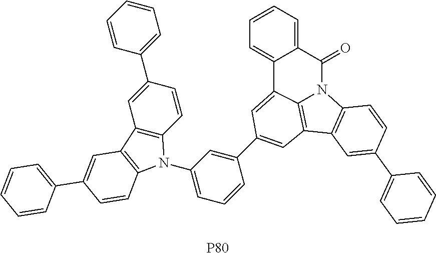 [SMILES: O=c1c2ccccc2c2cc(-c3cccc(-n4c5ccc(-c6ccccc6)cc5c5cc(-c6ccccc6)ccc54)c3)cc3c4cc(-c5ccccc5)ccc4n1c23 ; "P80"] |
| S114 | 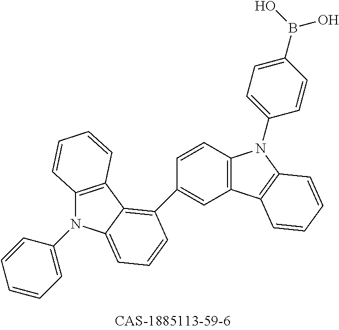 [SMILES: OB(O)c1ccc(-n2c3ccccc3c3cc(-c4cccc5c4c4ccccc4n5-c4ccccc4)ccc32)cc1] | 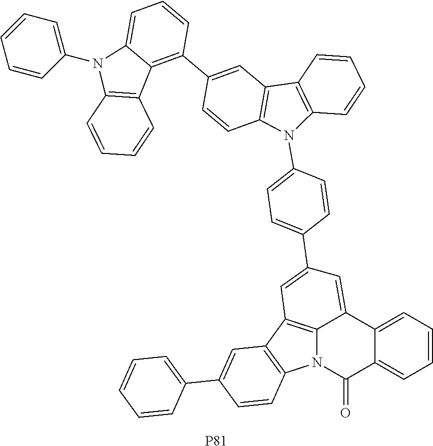 [SMILES: "P81" ; O=c1c2ccccc2c2cc(-c3ccc(-n4c5ccccc5c5cc(-c6cccc7c6c6ccccc6n7-c6ccccc6)ccc54)cc3)cc3c4cc(-c5ccccc5)ccc4n1c23] |
| S114 | 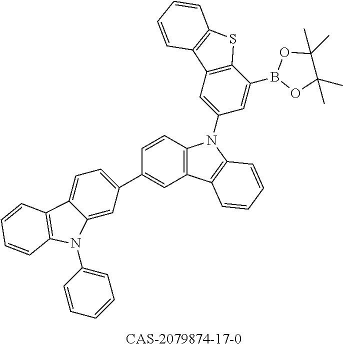 [SMILES: CC1(C)OB(c2cc(-n3c4ccccc4c4cc(-c5ccc6c7ccccc7n(-c7ccccc7)c6c5)ccc43)cc3c2sc2ccccc23)OC1(C)C] | 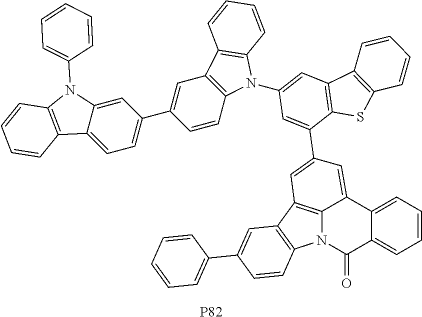 |
| S114 | 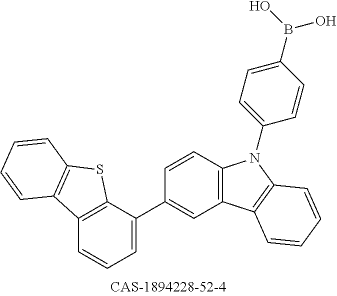 [SMILES: OB(O)c1ccc(-n2c3ccccc3c3cc(-c4cccc5c4sc4ccccc45)ccc32)cc1] | 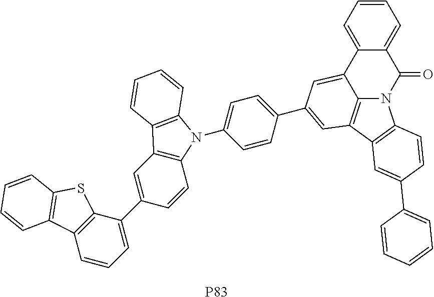 [SMILES: "P83" ; O=c1c2ccccc2c2cc(-c3ccc(-n4c5ccccc5c5cc(-c6cccc7c6sc6ccccc67)ccc54)cc3)cc3c4cc(-c5ccccc5)ccc4n1c23] |
| S115 | 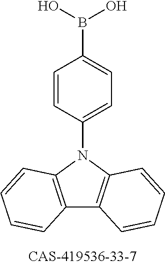 [SMILES: OB(O)c1ccc(-n2c3ccccc3c3ccccc32)cc1] | 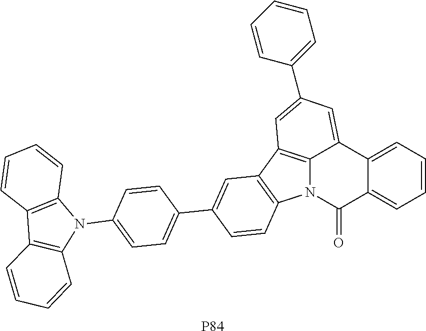 [SMILES: "P84" ; O=c1c2ccccc2c2cc(-c3ccccc3)cc3c4cc(-c5ccc(-n6c7ccccc7c7ccccc76)cc5)ccc4n1c23] |
| S115 | 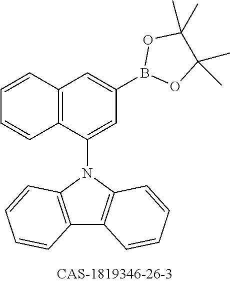 [SMILES: CC1(C)OB(c2cc(-n3c4ccccc4c4ccccc43)c3ccccc3c2)OC1(C)C] | 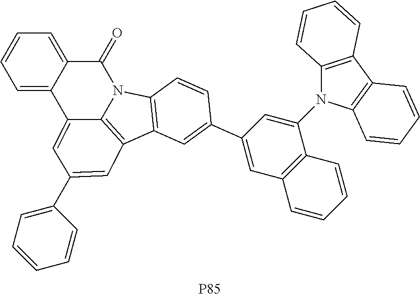 |
| S115 | 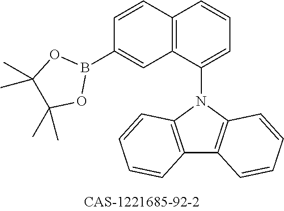 [SMILES: CC1(C)OB(c2ccc3cccc(-n4c5ccccc5c5ccccc54)c3c2)OC1(C)C] | 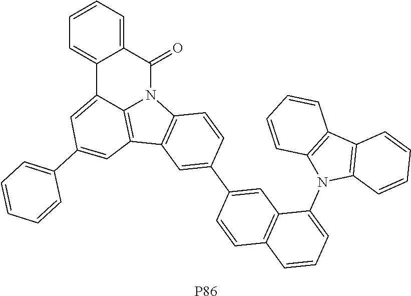 [SMILES: "P86" ; O=c1c2ccccc2c2cc(-c3ccccc3)cc3c4cc(-c5ccc6cccc(-n7c8ccccc8c8ccccc87)c6c5)ccc4n1c23] |
| S115 | 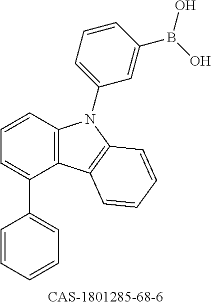 [SMILES: OB(O)c1cccc(-n2c3ccccc3c3c(-c4ccccc4)cccc32)c1] | 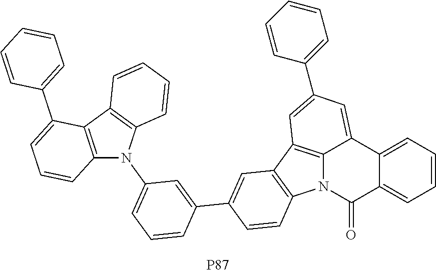 |
| S115 | 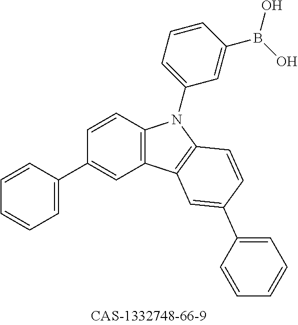 [SMILES: OB(O)c1cccc(-n2c3ccc(-c4ccccc4)cc3c3cc(-c4ccccc4)ccc32)c1] | 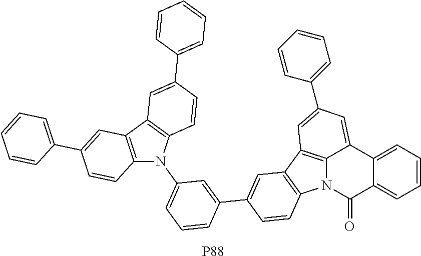 [SMILES: "P88" ; O=c1c2ccccc2c2cc(-c3ccccc3)cc3c4cc(-c5cccc(-n6c7ccc(-c8ccccc8)cc7c7cc(-c8ccccc8)ccc76)c5)ccc4n1c23] |
| S115 | 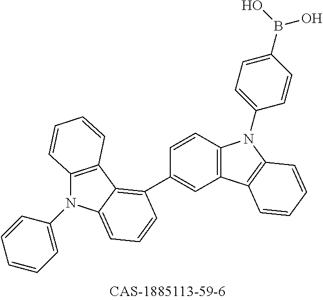 [SMILES: OB(O)c1ccc(-n2c3ccccc3c3cc(-c4cccc5c4c4ccccc4n5-c4ccccc4)ccc32)cc1] | 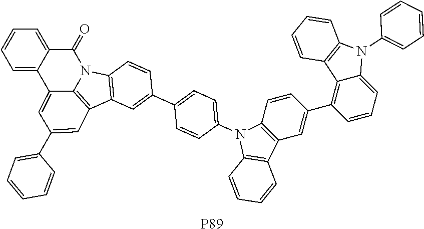 [SMILES: "P89" ; O=c1c2ccccc2c2cc(-c3ccccc3)cc3c4cc(-c5ccc(-n6c7ccccc7c7cc(-c8cccc9c8c8ccccc8n9-c8ccccc8)ccc76)cc5)ccc4n1c23] |
| S115 | 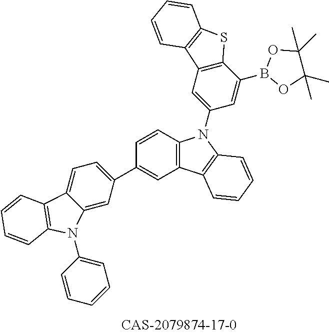 [SMILES: CC1(C)OB(c2cc(-n3c4ccccc4c4cc(-c5ccc6c7ccccc7n(-c7ccccc7)c6c5)ccc43)cc3c2sc2ccccc23)OC1(C)C] | 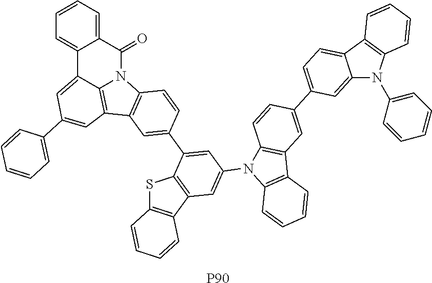 [SMILES: "P90" ; O=c1c2ccccc2c2cc(-c3ccccc3)cc3c4cc(-c5cc(-n6c7ccccc7c7cc(-c8ccc9c%10ccccc%10n(-c%10ccccc%10)c9c8)ccc76)cc6c5sc5ccccc56)ccc4n1c23] |
| S115 | 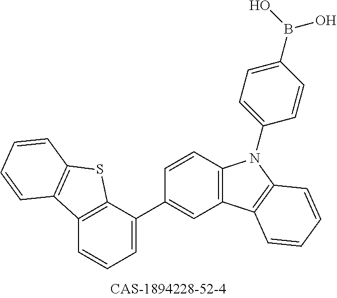 [SMILES: OB(O)c1ccc(-n2c3ccccc3c3cc(-c4cccc5c4sc4ccccc45)ccc32)cc1] | 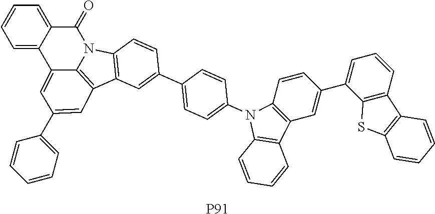 [SMILES: "P91" ; O=c1c2ccccc2c2cc(-c3ccccc3)cc3c4cc(-c5ccc(-n6c7ccccc7c7cc(-c8cccc9c8sc8ccccc89)ccc76)cc5)ccc4n1c23] |

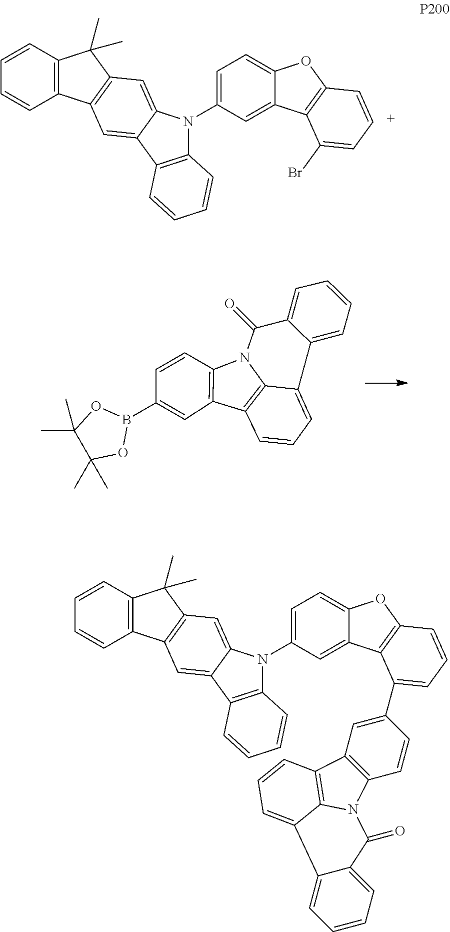 [SMILES: CC1(C)c2ccccc2-c2cc3c4ccccc4n(-c4ccc5oc6cccc(Br)c6c5c4)c3cc21 ; CC1(C)OB(c2ccc3c(c2)c2cccc4c5ccccc5c(=O)n3c42)OC1(C)C ; CC1(C)c2ccccc2-c2cc3c4ccccc4n(-c4ccc5oc6cccc(-c7ccc8c(c7)c7cccc9c%10ccccc%10c(=O)n8c97)c6c5c4)c3cc21]

An initial charge of 5-(9-bromo-2-dibenzofuranyl)-5,7-dihydro-7,7-dimethylindeno[2,1-b]carbazole [2226483-41-4] (21.24 g, 40.2 mmol), S125 (15.89 g, 40.2 mmol) and sodium carbonate (8.51 g, 80.3 mmol) in toluene (200 ml), 1,4-dioxane (200 ml) and water (100 ml) is inertized with argon for 20 min. Subsequently, tetrakis(triphenylphosphine)palladium(0) (928 mg, 0.80 mmol) is added and the reaction mixture is stirred under reflux for 32 h. After cooling, the precipitated solids are filtered off with suction and washed with ethanol. The crude product is twice subjected to hot extraction with toluene, then recrystallized three times from dimethylacetamide and finally sublimed under high vacuum. Yield: 15.8 g (22.1 mmol, 55%) of yellow solid, purity: >99.9% by HPLC.

Catalyst System for the Conversion of Cl Rather than Br:

For the conversion of Cl rather than bromine, the phosphine ligand used is X-Phos [564483-18-7] or S-Phos [657408-07-6] rather than tetrakis(triphenylphosphine)palladium(0), or the palladium source used is Pd(OAc)₂ [3375-31-3] or Pd₂(dba)₃ [51364-51-3]. Alternatively, the catalyst system used may also be Pd—X-Phos-G3 [1445085-55-1]. It may also be advantageous for the conversion of bromine to use one of the latter catalyst systems.

Purification can also be effected using column chromatography, or recrystallization or hot extraction using other standard solvents such as ethanol, butanol, acetone, ethyl acetate, acetonitrile, toluene, xylene, dichloromethane, methanol, tetrahydrofuran, n-butyl acetate, 1,4-dioxane, or recrystallization using high boilers such as dimethyl sulfoxide, N,N-dimethylformamide, N,N-dimethylacetamide, N-methylpyrrolidone, etc. The yields are typically in the range between 13% and 75%.

| Reactant | | |
|---|---|---|
| 1 | Reactant 2 | Product |
| S125 | 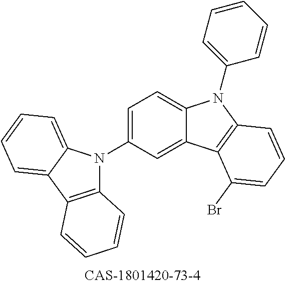 [SMILES: Brc1cccc2c1c1cc(-n3c4ccccc4c4ccccc43)ccc1n2-c1ccccc1] | 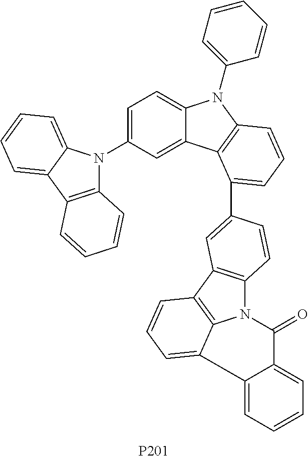 [SMILES: "P201" ; O=c1c2ccccc2c2cccc3c4cc(-c5cccc6c5c5cc(-n7c8ccccc8c8ccccc87)ccc5n6-c5ccccc5)ccc4n1c23] |
| S125 | 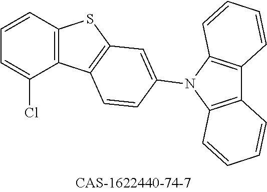 [SMILES: Clc1cccc2sc3cc(-n4c5ccccc5c5ccccc54)ccc3c12] | 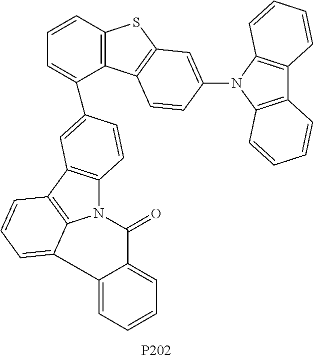 [SMILES: "P202" ; O=c1c2ccccc2c2cccc3c4cc(-c5cccc6sc7cc(-n8c9ccccc9c9ccccc98)ccc7c56)ccc4n1c23] |
| S125 | 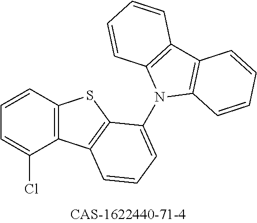 [SMILES: Clc1cccc2sc3c(-n4c5ccccc5c5ccccc54)cccc3c12] | 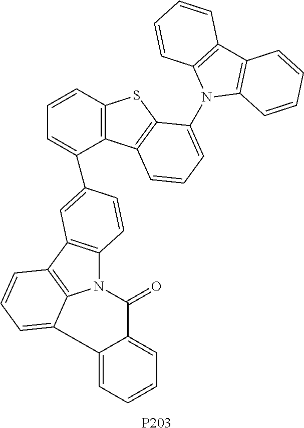 [SMILES: "P203" ; O=c1c2ccccc2c2cccc3c4cc(-c5cccc6sc7c(-n8c9ccccc9c9ccccc98)cccc7c56)ccc4n1c23] |
| S125 | 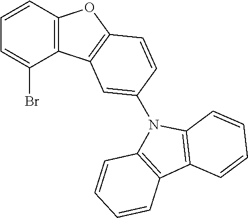 [SMILES: Brc1cccc2oc3ccc(-n4c5ccccc5c5ccccc54)cc3c12] | 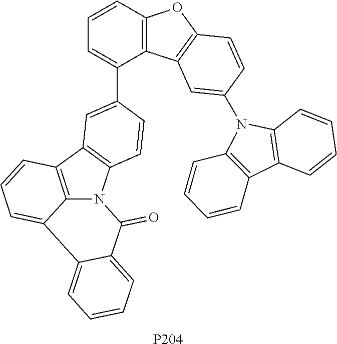 [SMILES: "P204" ; O=c1c2ccccc2c2cccc3c4cc(-c5cccc6oc7ccc(-n8c9ccccc9c9ccccc98)cc7c56)ccc4n1c23] |
| S125 | 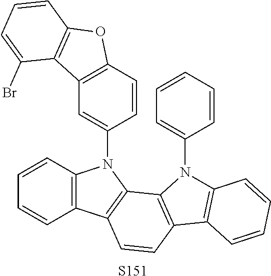 [SMILES: Brc1cccc2oc3ccc(-n4c5ccccc5c5ccc6c7ccccc7n(-c7ccccc7)c6c54)cc3c12 ; "S151"] | 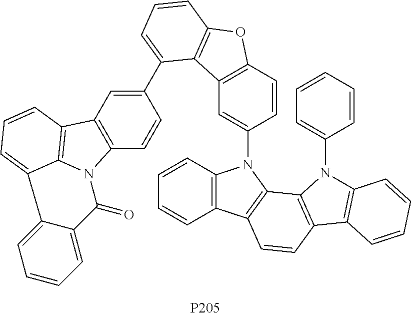 [SMILES: "P205" ; O=c1c2ccccc2c2cccc3c4cc(-c5cccc6oc7ccc(-n8c9ccccc9c9ccc%10c%11ccccc%11n(-c%11ccccc%11)c%10c98)cc7c56)ccc4n1c23] |
| S125 | 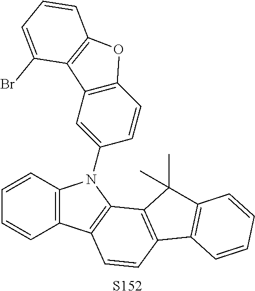 [SMILES: CC1(C)c2ccccc2-c2ccc3c4ccccc4n(-c4ccc5oc6cccc(Br)c6c5c4)c3c21 ; "S152"] | 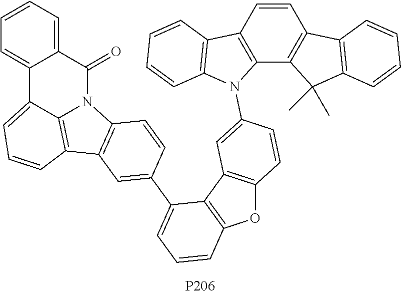 [SMILES: "P206" ; CC1(C)c2ccccc2-c2ccc3c4ccccc4n(-c4ccc5oc6cccc(-c7ccc8c(c7)c7cccc9c%10ccccc%10c(=O)n8c97)c6c5c4)c3c21] |
| S125 | 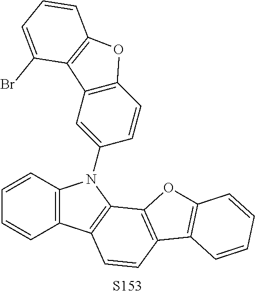 [SMILES: Brc1cccc2oc3ccc(-n4c5ccccc5c5ccc6c7ccccc7oc6c54)cc3c12 ; "S153"] | 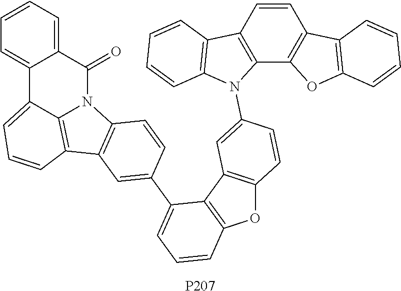 [SMILES: "P207" ; O=c1c2ccccc2c2cccc3c4cc(-c5cccc6oc7ccc(-n8c9ccccc9c9ccc%10c%11ccccc%11oc%10c98)cc7c56)ccc4n1c23] |
| S125 | 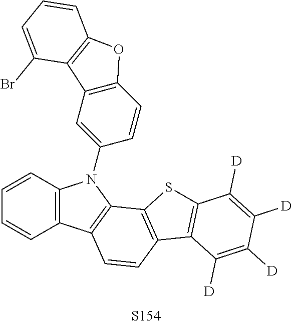 [SMILES: [2H]c1c([2H])c([2H])c2c(sc3c2ccc2c4ccccc4n(-c4ccc5oc6cccc(Br)c6c5c4)c23)c1[2H] ; "S154"] | 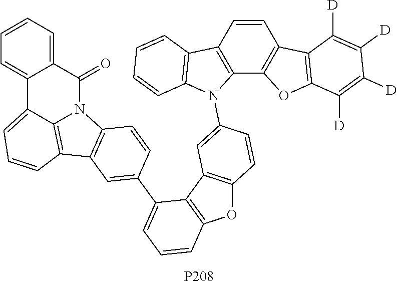 [SMILES: "P208" ; [2H]c1c([2H])c([2H])c2c(oc3c2ccc2c4ccccc4n(-c4ccc5oc6cccc(-c7ccc8c(c7)c7cccc9c%10ccccc%10c(=O)n8c97)c6c5c4)c23)c1[2H]] |
| S125 | 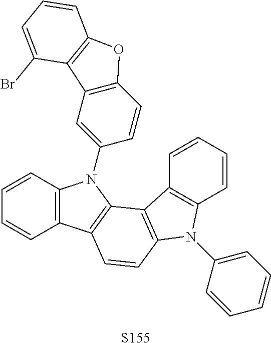 [SMILES: Brc1cccc2oc3ccc(-n4c5ccccc5c5ccc6c(c7ccccc7n6-c6ccccc6)c54)cc3c12 ; "S155"] | 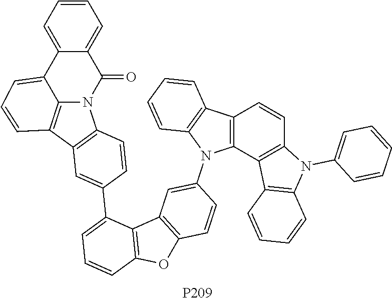 [SMILES: "P209" ; O=c1c2ccccc2c2cccc3c4cc(-c5cccc6oc7ccc(-n8c9ccccc9c9ccc%10c(c%11ccccc%11n%10-c%10ccccc%10)c98)cc7c56)ccc4n1c23] |
| S125 | 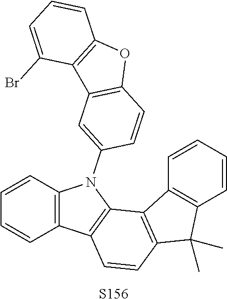 [SMILES: CC1(C)c2ccccc2-c2c1ccc1c3ccccc3n(-c3ccc4oc5cccc(Br)c5c4c3)c21 ; "S156"] | 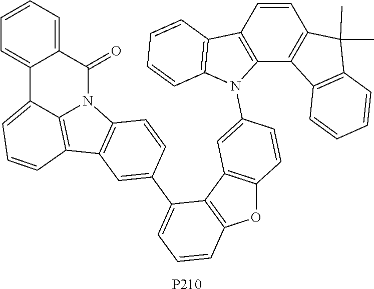 [SMILES: "P210" ; CC1(C)c2ccccc2-c2c1ccc1c3ccccc3n(-c3ccc4oc5cccc(-c6ccc7c(c6)c6cccc8c9ccccc9c(=O)n7c86)c5c4c3)c21] |
| S125 | 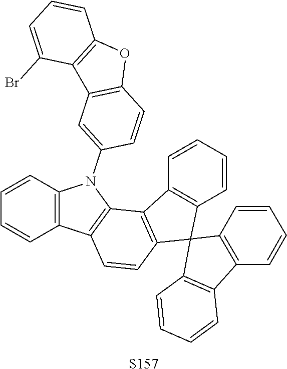 [SMILES: Brc1cccc2oc3ccc(-n4c5ccccc5c5ccc6c(c54)-c4ccccc4C64c5ccccc5-c5ccccc54)cc3c12 ; "S157"] | 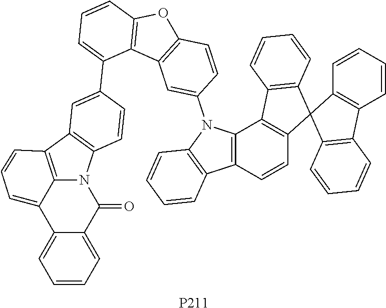 [SMILES: "P211" ; O=c1c2ccccc2c2cccc3c4cc(-c5cccc6oc7ccc(-n8c9ccccc9c9ccc%10c(c98)-c8ccccc8C%108c9ccccc9-c9ccccc98)cc7c56)ccc4n1c23] |
| S125 | 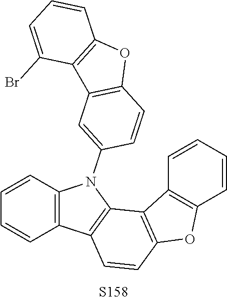 [SMILES: Brc1cccc2oc3ccc(-n4c5ccccc5c5ccc6oc7ccccc7c6c54)cc3c12 ; "S158"] | 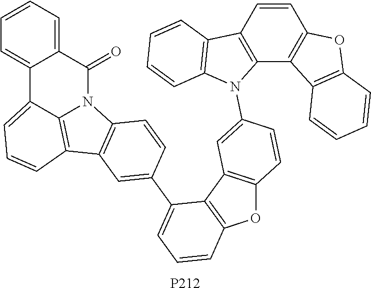 [SMILES: "P212" ; O=c1c2ccccc2c2cccc3c4cc(-c5cccc6oc7ccc(-n8c9ccccc9c9ccc%10oc%11ccccc%11c%10c98)cc7c56)ccc4n1c23] |
| S125 | 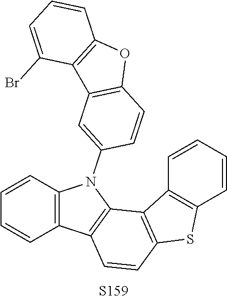 [SMILES: Brc1cccc2oc3ccc(-n4c5ccccc5c5ccc6sc7ccccc7c6c54)cc3c12 ; "S159"] | 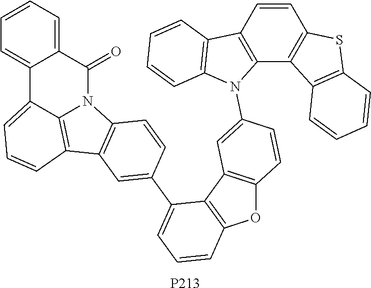 [SMILES: "P213" ; O=c1c2ccccc2c2cccc3c4cc(-c5cccc6oc7ccc(-n8c9ccccc9c9ccc%10sc%11ccccc%11c%10c98)cc7c56)ccc4n1c23] |
| S125 | 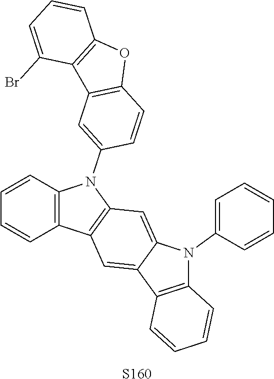 [SMILES: Brc1cccc2oc3ccc(-n4c5ccccc5c5cc6c7ccccc7n(-c7ccccc7)c6cc54)cc3c12 ; "S160"] | 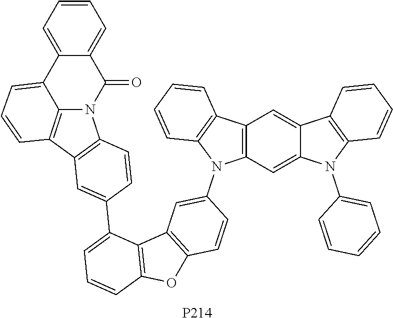 [SMILES: "P214" ; O=c1c2ccccc2c2cccc3c4cc(-c5cccc6oc7ccc(-n8c9ccccc9c9cc%10c%11ccccc%11n(-c%11ccccc%11)c%10cc98)cc7c56)ccc4n1c23] |
| S125 | 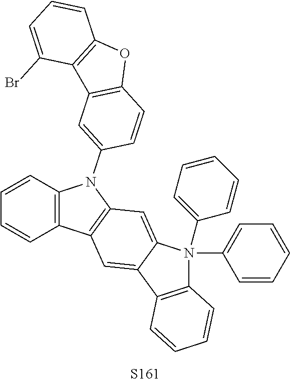 | 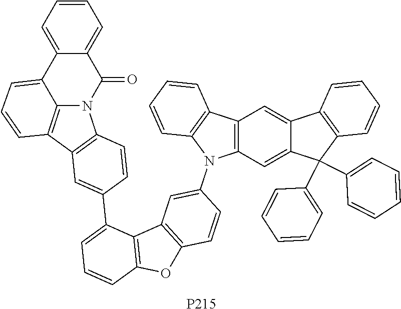 [SMILES: "P215" ; O=c1c2ccccc2c2cccc3c4cc(-c5cccc6oc7ccc(-n8c9ccccc9c9cc%10c(cc98)C(c8ccccc8)(c8ccccc8)c8ccccc8-%10)cc7c56)ccc4n1c23] |
| S125 | 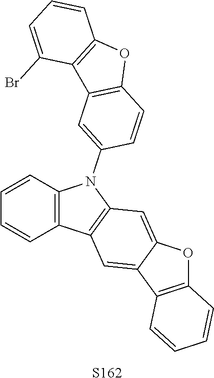 [SMILES: Brc1cccc2oc3ccc(-n4c5ccccc5c5cc6c(cc54)oc4ccccc46)cc3c12 ; "S162"] | 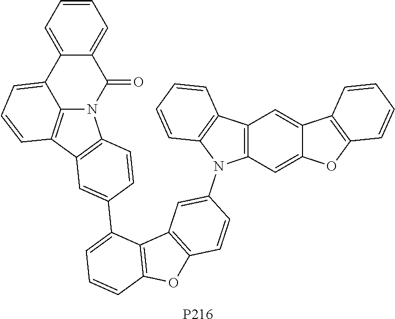 [SMILES: "P216" ; O=c1c2ccccc2c2cccc3c4cc(-c5cccc6oc7ccc(-n8c9ccccc9c9cc%10c(cc98)oc8ccccc8%10)cc7c56)ccc4n1c23] |
| S125 | 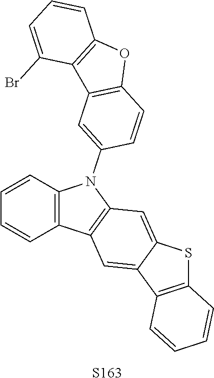 [SMILES: Brc1cccc2oc3ccc(-n4c5ccccc5c5cc6c(cc54)sc4ccccc46)cc3c12 ; "S163"] | 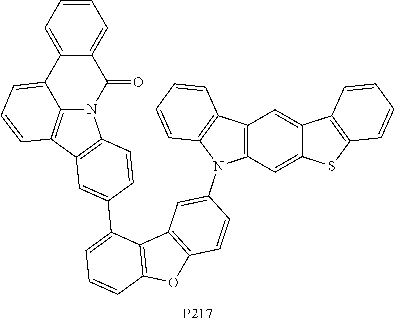 [SMILES: "P217" ; O=c1c2ccccc2c2cccc3c4cc(-c5cccc6oc7ccc(-n8c9ccccc9c9cc%10c(cc98)sc8ccccc8%10)cc7c56)ccc4n1c23] |
| S125 | 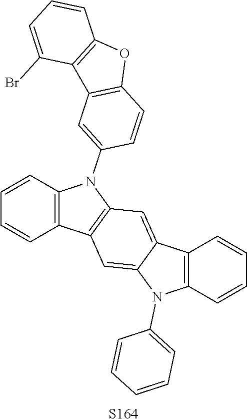 [SMILES: Brc1cccc2oc3ccc(-n4c5ccccc5c5cc6c(cc54)c4ccccc4n6-c4ccccc4)cc3c12 ; "S164"] | 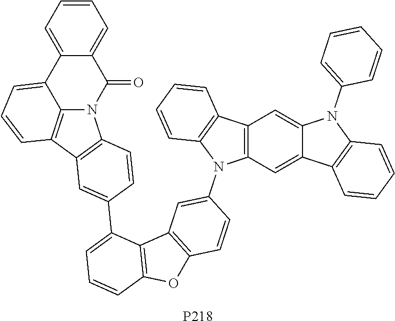 [SMILES: "P218" ; O=c1c2ccccc2c2cccc3c4cc(-c5cccc6oc7ccc(-n8c9ccccc9c9cc%10c(cc98)c8ccccc8n%10-c8ccccc8)cc7c56)ccc4n1c23] |
| S125 | 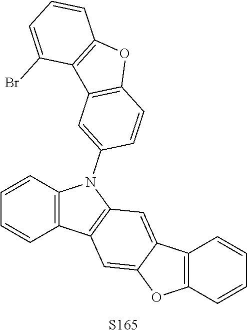 [SMILES: Brc1cccc2oc3ccc(-n4c5ccccc5c5cc6oc7ccccc7c6cc54)cc3c12 ; "S165"] | 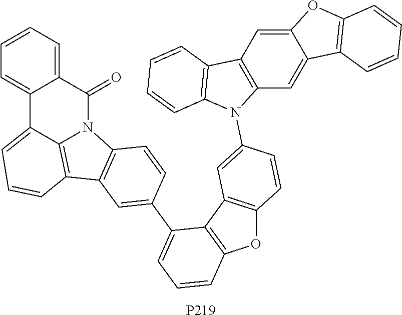 [SMILES: "P219" ; O=c1c2ccccc2c2cccc3c4cc(-c5cccc6oc7ccc(-n8c9ccccc9c9cc%10oc%11ccccc%11c%10cc98)cc7c56)ccc4n1c23] |
| S125 | 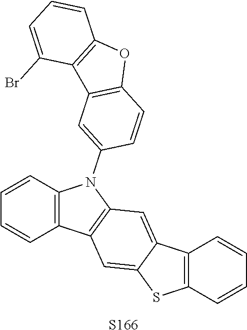 [SMILES: Brc1cccc2oc3ccc(-n4c5ccccc5c5cc6sc7ccccc7c6cc54)cc3c12 ; "S166"] | 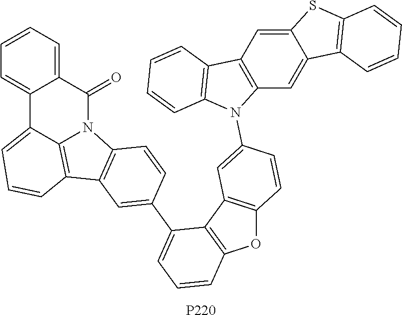 [SMILES: "P220" ; O=c1c2ccccc2c2cccc3c4cc(-c5cccc6oc7ccc(-n8c9ccccc9c9cc%10sc%11ccccc%11c%10cc98)cc7c56)ccc4n1c23] |
| S125 | 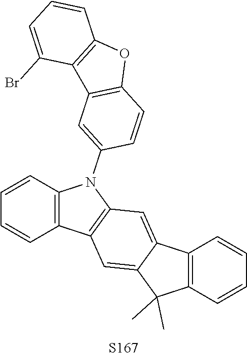 [SMILES: CC1(C)c2ccccc2-c2cc3c(cc21)c1ccccc1n3-c1ccc2oc3cccc(Br)c3c2c1 ; "S167"] | 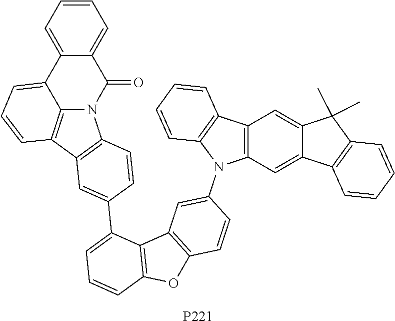 [SMILES: "P221" ; CC1(C)c2ccccc2-c2cc3c(cc21)c1ccccc1n3-c1ccc2oc3cccc(-c4ccc5c(c4)c4cccc6c7ccccc7c(=O)n5c64)c3c2c1] |
| S125 | 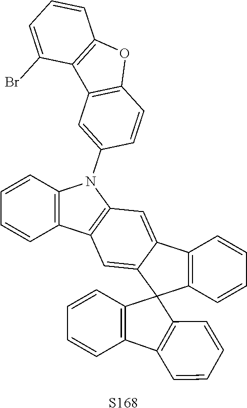 [SMILES: Brc1cccc2oc3ccc(-n4c5ccccc5c5cc6c(cc54)-c4ccccc4C64c5ccccc5-c5ccccc54)cc3c12 ; "S168"] | 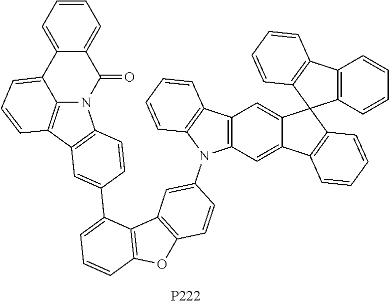 [SMILES: "P222" ; O=c1c2ccccc2c2cccc3c4cc(-c5cccc6oc7ccc(-n8c9ccccc9c9cc%10c(cc98)-c8ccccc8C%108c9ccccc9-c9ccccc98)cc7c56)ccc4n1c23] |
| S125 | 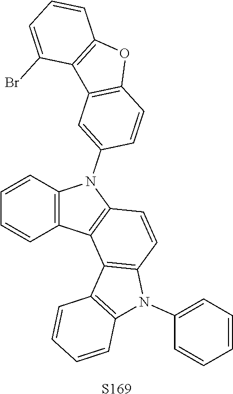 [SMILES: Brc1cccc2oc3ccc(-n4c5ccccc5c5c6c7ccccc7n(-c7ccccc7)c6ccc54)cc3c12 ; "S169"] | 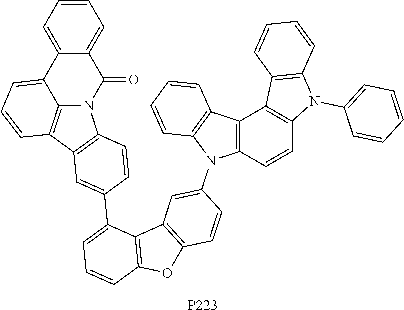 [SMILES: "P223" ; O=c1c2ccccc2c2cccc3c4cc(-c5cccc6oc7ccc(-n8c9ccccc9c9c%10c%11ccccc%11n(-c%11ccccc%11)c%10ccc98)cc7c56)ccc4n1c23] |
| S125 | 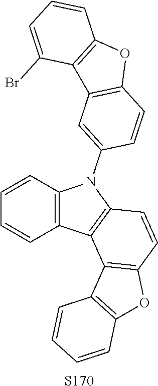 [SMILES: Brc1cccc2oc3ccc(-n4c5ccccc5c5c6c(ccc54)oc4ccccc46)cc3c12 ; "S170"] | 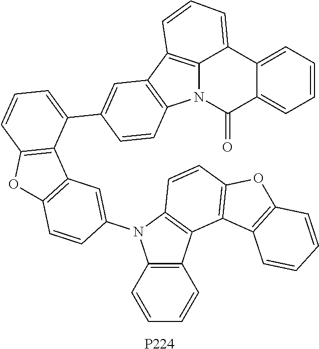 [SMILES: "P224" ; O=c1c2ccccc2c2cccc3c4cc(-c5cccc6oc7ccc(-n8c9ccccc9c9c%10c(ccc98)oc8ccccc8%10)cc7c56)ccc4n1c23] |
| S125 | 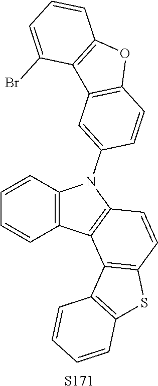 [SMILES: Brc1cccc2oc3ccc(-n4c5ccccc5c5c6c(ccc54)sc4ccccc46)cc3c12 ; "S171"] | 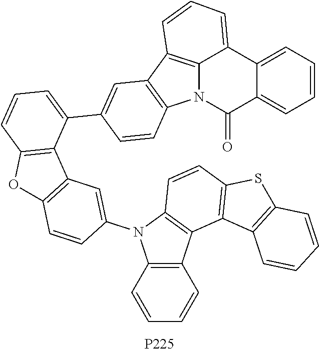 [SMILES: "P225" ; O=c1c2ccccc2c2cccc3c4cc(-c5cccc6oc7ccc(-n8c9ccccc9c9c%10c(ccc98)sc8ccccc8%10)cc7c56)ccc4n1c23] |
| S125 | 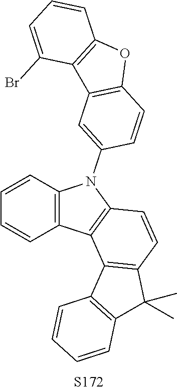 [SMILES: CC1(C)c2ccccc2-c2c1ccc1c2c2ccccc2n1-c1ccc2oc3cccc(Br)c3c2c1 ; "S172"] | 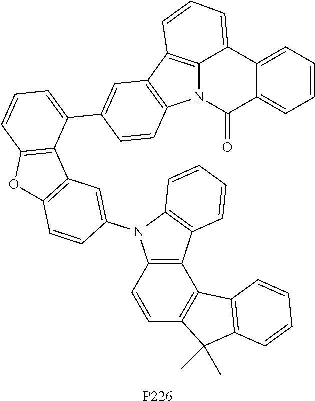 [SMILES: "P226" ; CC1(C)c2ccccc2-c2c1ccc1c2c2ccccc2n1-c1ccc2oc3cccc(-c4ccc5c(c4)c4cccc6c7ccccc7c(=O)n5c64)c3c2c1] |
| S125 | 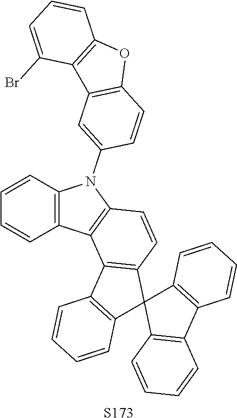 [SMILES: Brc1cccc2oc3ccc(-n4c5ccccc5c5c6c(ccc54)C4(c5ccccc5-c5ccccc54)c4ccccc4-6)cc3c12 ; "S173"] | 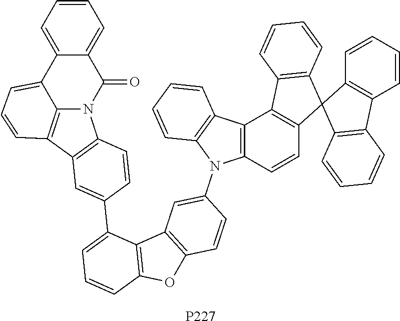 [SMILES: "P227" ; O=c1c2ccccc2c2cccc3c4cc(-c5cccc6oc7ccc(-n8c9ccccc9c9c%10c(ccc98)C8(c9ccccc9-c9ccccc98)c8ccccc8-%10)cc7c56)ccc4n1c23] |
| S125 | 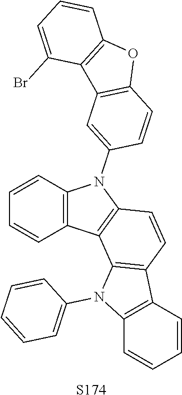 [SMILES: Brc1cccc2oc3ccc(-n4c5ccccc5c5c4ccc4c6ccccc6n(-c6ccccc6)c45)cc3c12 ; "S174"] | 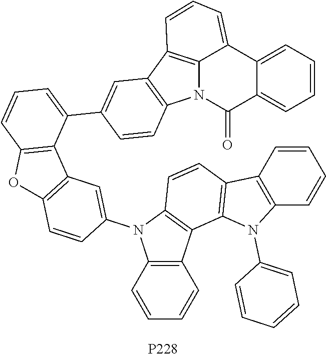 [SMILES: "P228" ; O=c1c2ccccc2c2cccc3c4cc(-c5cccc6oc7ccc(-n8c9ccccc9c9c8ccc8c%10ccccc%10n(-c%10ccccc%10)c89)cc7c56)ccc4n1c23] |
| S125 | 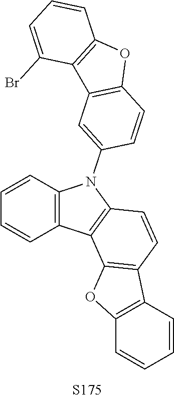 [SMILES: Brc1cccc2oc3ccc(-n4c5ccccc5c5c6oc7ccccc7c6ccc54)cc3c12 ; "S175"] | 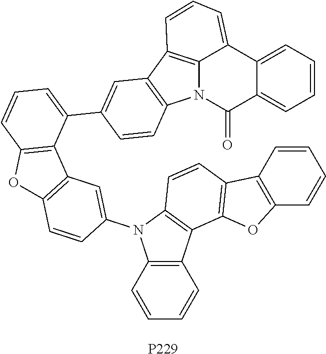 [SMILES: "P229" ; O=c1c2ccccc2c2cccc3c4cc(-c5cccc6oc7ccc(-n8c9ccccc9c9c%10oc%11ccccc%11c%10ccc98)cc7c56)ccc4n1c23] |
| S125 | 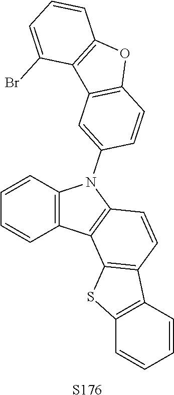 [SMILES: Brc1cccc2oc3ccc(-n4c5ccccc5c5c6sc7ccccc7c6ccc54)cc3c12 ; "S176"] | 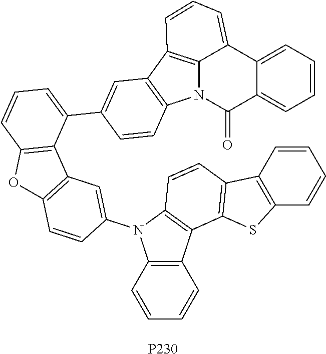 [SMILES: "P230" ; O=c1c2ccccc2c2cccc3c4cc(-c5cccc6oc7ccc(-n8c9ccccc9c9c%10sc%11ccccc%11c%10ccc98)cc7c56)ccc4n1c23] |
| S125 | 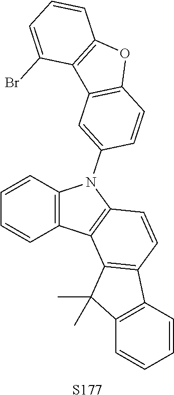 [SMILES: CC1(C)c2ccccc2-c2ccc3c(c21)c1ccccc1n3-c1ccc2oc3cccc(Br)c3c2c1 ; "S177"] | 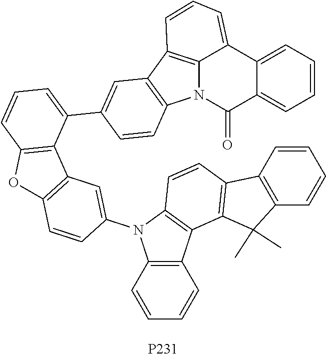 [SMILES: "P231" ; CC1(C)c2ccccc2-c2ccc3c(c21)c1ccccc1n3-c1ccc2oc3cccc(-c4ccc5c(c4)c4cccc6c7ccccc7c(=O)n5c64)c3c2c1] |
| S125 | 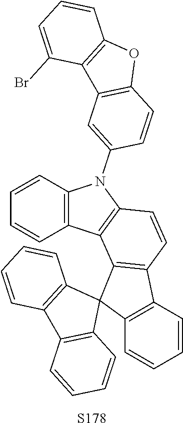 [SMILES: Brc1cccc2oc3ccc(-n4c5ccccc5c5c6c(ccc54)-c4ccccc4C64c5ccccc5-c5ccccc54)cc3c12 ; "S178"] | 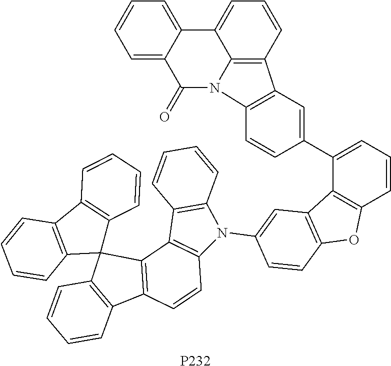 [SMILES: "P232" ; O=c1c2ccccc2c2cccc3c4cc(-c5cccc6oc7ccc(-n8c9ccccc9c9c%10c(ccc98)-c8ccccc8C%108c9ccccc9-c9ccccc98)cc7c56)ccc4n1c23] |
| S125 | 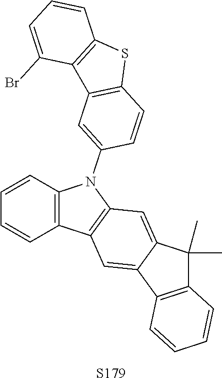 [SMILES: CC1(C)c2ccccc2-c2cc3c4ccccc4n(-c4ccc5sc6cccc(Br)c6c5c4)c3cc21 ; "S179"] | 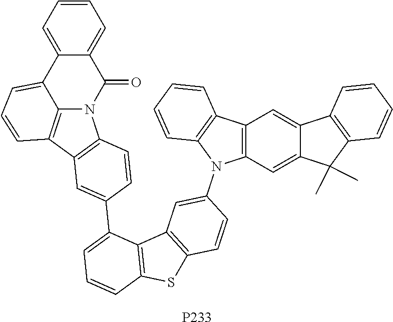 [SMILES: "P233" ; CC1(C)c2ccccc2-c2cc3c4ccccc4n(-c4ccc5sc6cccc(-c7ccc8c(c7)c7cccc9c%10ccccc%10c(=O)n8c97)c6c5c4)c3cc21] |
| S125 | 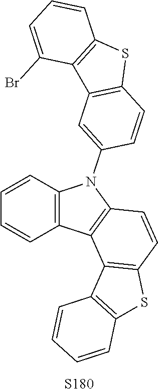 [SMILES: Brc1cccc2sc3ccc(-n4c5ccccc5c5c6c(ccc54)sc4ccccc46)cc3c12 ; "S180"] | 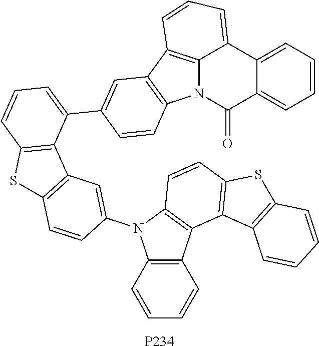 [SMILES: "P234" ; O=c1c2ccccc2c2cccc3c4cc(-c5cccc6sc7ccc(-n8c9ccccc9c9c%10c(ccc98)sc8ccccc8%10)cc7c56)ccc4n1c23] |
| S125 | 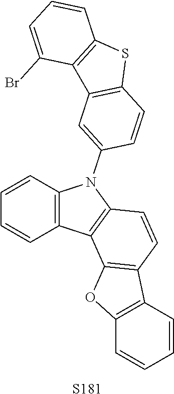 [SMILES: Brc1cccc2sc3ccc(-n4c5ccccc5c5c6oc7ccccc7c6ccc54)cc3c12 ; "S181"] | 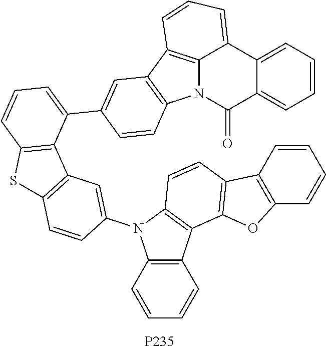 [SMILES: "P235" ; O=c1c2ccccc2c2cccc3c4cc(-c5cccc6sc7ccc(-n8c9ccccc9c9c%10oc%11ccccc%11c%10ccc98)cc7c56)ccc4n1c23] |
| S125 | 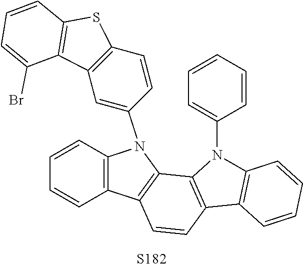 [SMILES: Brc1cccc2sc3ccc(-n4c5ccccc5c5ccc6c7ccccc7n(-c7ccccc7)c6c54)cc3c12 ; "S182"] | 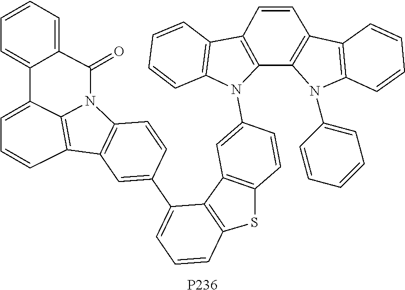 [SMILES: "P236" ; O=c1c2ccccc2c2cccc3c4cc(-c5cccc6sc7ccc(-n8c9ccccc9c9ccc%10c%11ccccc%11n(-c%11ccccc%11)c%10c98)cc7c56)ccc4n1c23] |
| S125 | 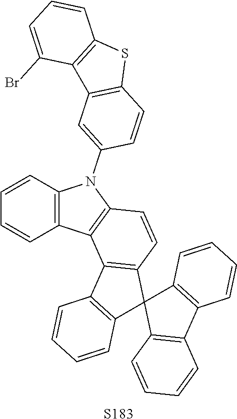 [SMILES: Brc1cccc2sc3ccc(-n4c5ccccc5c5c6c(ccc54)C4(c5ccccc5-c5ccccc54)c4ccccc4-6)cc3c12 ; "S183"] | 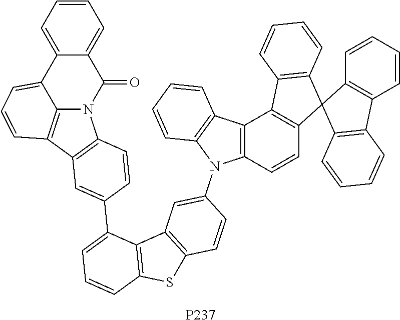 [SMILES: "P237" ; O=c1c2ccccc2c2cccc3c4cc(-c5cccc6sc7ccc(-n8c9ccccc9c9c%10c(ccc98)C8(c9ccccc9-c9ccccc98)c8ccccc8-%10)cc7c56)ccc4n1c23] |
| S125 | 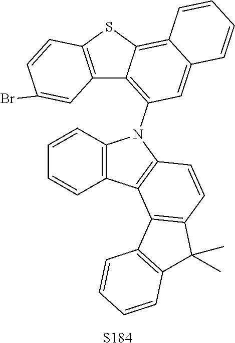 [SMILES: CC1(C)c2ccccc2-c2c1ccc1c2c2ccccc2n1-c1cc2ccccc2c2sc3ccc(Br)cc3c12 ; "S184"] | 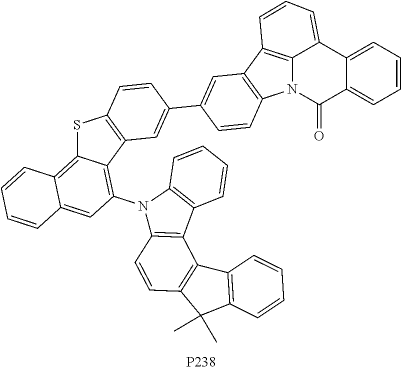 [SMILES: "P238" ; CC1(C)c2ccccc2-c2c1ccc1c2c2ccccc2n1-c1cc2ccccc2c2sc3ccc(-c4ccc5c(c4)c4cccc6c7ccccc7c(=O)n5c64)cc3c12] |
| S125 | 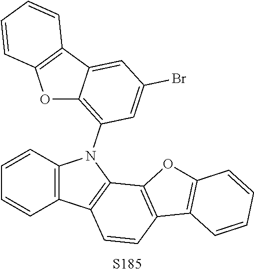 [SMILES: Brc1cc(-n2c3ccccc3c3ccc4c5ccccc5oc4c32)c2oc3ccccc3c2c1 ; "S185"] | 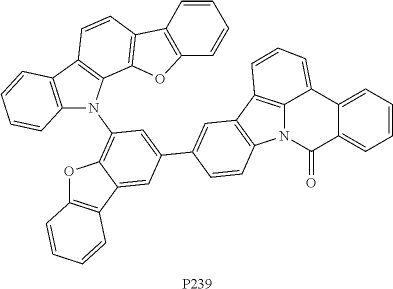 [SMILES: "P239" ; O=c1c2ccccc2c2cccc3c4cc(-c5cc(-n6c7ccccc7c7ccc8c9ccccc9oc8c76)c6oc7ccccc7c6c5)ccc4n1c23] |
| S125 | 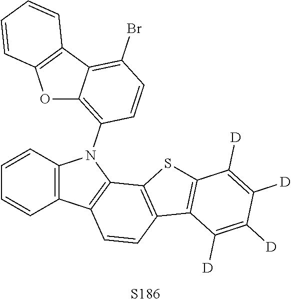 [SMILES: [2H]c1c([2H])c([2H])c2c(sc3c2ccc2c4ccccc4n(-c4ccc(Br)c5c4oc4ccccc45)c23)c1[2H] ; "S186"] | 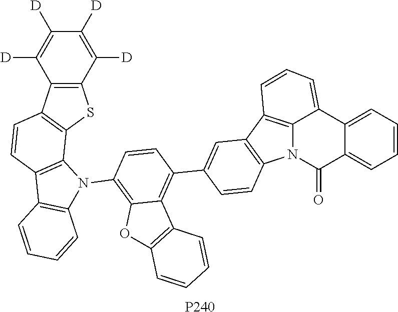 [SMILES: "P240" ; [2H]c1c([2H])c([2H])c2c(sc3c2ccc2c4ccccc4n(-c4ccc(-c5ccc6c(c5)c5cccc7c8ccccc8c(=O)n6c75)c5c4oc4ccccc45)c23)c1[2H]] |
| S125 | 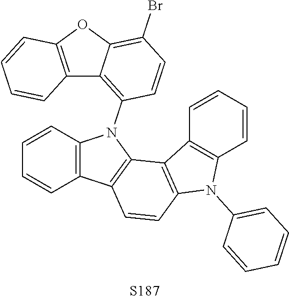 [SMILES: Brc1ccc(-n2c3ccccc3c3ccc4c(c5ccccc5n4-c4ccccc4)c32)c2c1oc1ccccc12 ; "S187"] | 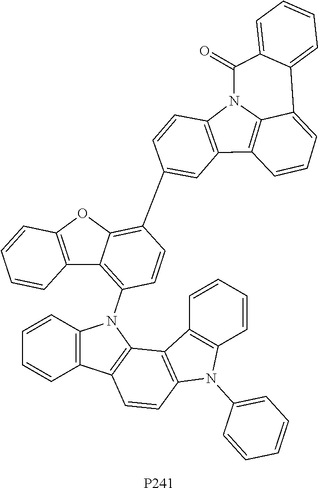 [SMILES: "P241" ; O=c1c2ccccc2c2cccc3c4cc(-c5ccc(-n6c7ccccc7c7ccc8c(c9ccccc9n8-c8ccccc8)c76)c6c5oc5ccccc56)ccc4n1c23] |
| S125 | 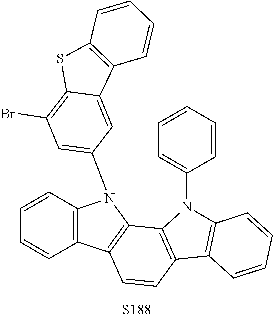 [SMILES: Brc1cc(-n2c3ccccc3c3ccc4c5ccccc5n(-c5ccccc5)c4c32)cc2c1sc1ccccc12 ; "S188"] | 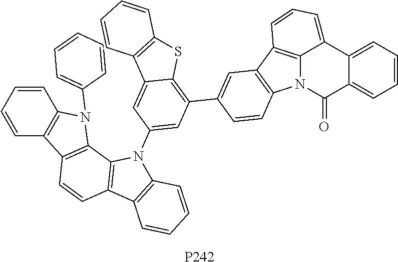 [SMILES: "P242" ; O=c1c2ccccc2c2cccc3c4cc(-c5cc(-n6c7ccccc7c7ccc8c9ccccc9n(-c9ccccc9)c8c76)cc6c5sc5ccccc56)ccc4n1c23] |
| S125 | 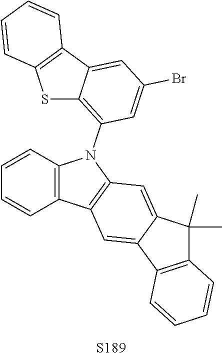 [SMILES: CC1(C)c2ccccc2-c2cc3c4ccccc4n(-c4cc(Br)cc5c4sc4ccccc45)c3cc21 ; "S189"] | 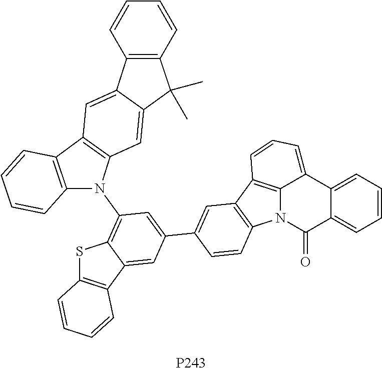 [SMILES: "P243" ; CC1(C)c2ccccc2-c2cc3c4ccccc4n(-c4cc(-c5ccc6c(c5)c5cccc7c8ccccc8c(=O)n6c75)cc5c4sc4ccccc45)c3cc21] |
| S125 | 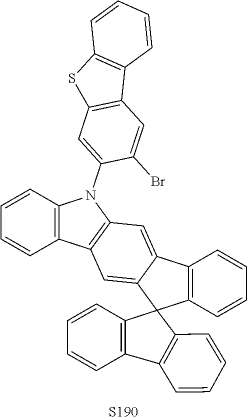 [SMILES: Brc1cc2c(cc1-n1c3ccccc3c3cc4c(cc31)-c1ccccc1C41c3ccccc3-c3ccccc31)sc1ccccc12 ; "S190"] | 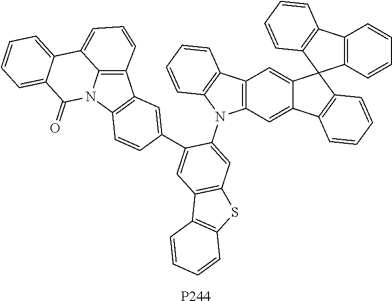 [SMILES: "P244" ; O=c1c2ccccc2c2cccc3c4cc(-c5cc6c(cc5-n5c7ccccc7c7cc8c(cc75)-c5ccccc5C85c7ccccc7-c7ccccc75)sc5ccccc56)ccc4n1c23] |
| S125 | 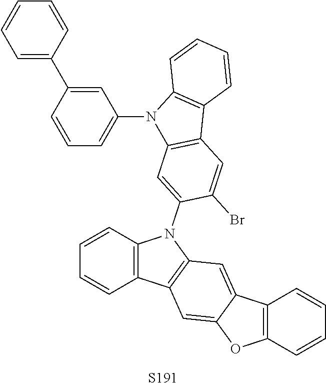 [SMILES: Brc1cc2c3ccccc3n(-c3cccc(-c4ccccc4)c3)c2cc1-n1c2ccccc2c2cc3oc4ccccc4c3cc21 ; "S191"] | 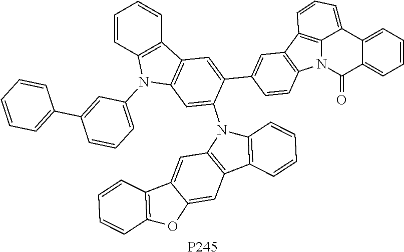 [SMILES: "P245" ; O=c1c2ccccc2c2cccc3c4cc(-c5cc6c7ccccc7n(-c7cccc(-c8ccccc8)c7)c6cc5-n5c6ccccc6c6cc7oc8ccccc8c7cc65)ccc4n1c23] |
| S125 | 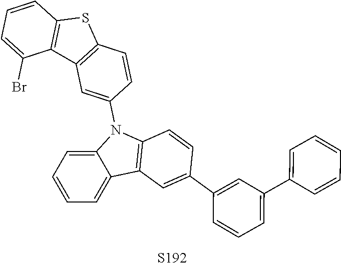 [SMILES: Brc1cccc2sc3ccc(-n4c5ccccc5c5cc(-c6cccc(-c7ccccc7)c6)ccc54)cc3c12 ; "S192"] | 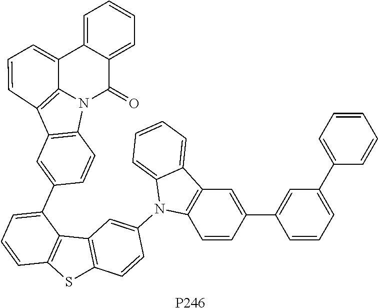 [SMILES: "P246" ; O=c1c2ccccc2c2cccc3c4cc(-c5cccc6sc7ccc(-n8c9ccccc9c9cc(-c%10cccc(-c%11ccccc%11)c%10)ccc98)cc7c56)ccc4n1c23] |
| S125 | 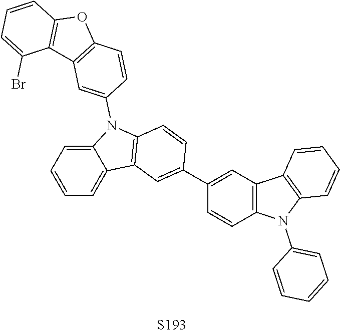 [SMILES: "S193" ; Brc1cccc2oc3ccc(-n4c5ccccc5c5cc(-c6ccc7c(c6)c6ccccc6n7-c6ccccc6)ccc54)cc3c12] | 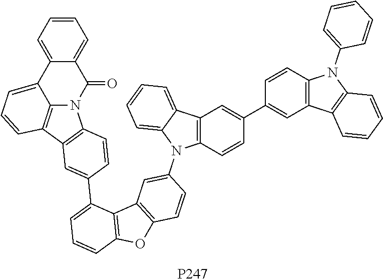 |
| S125 | 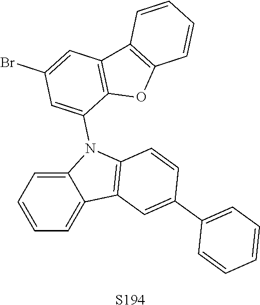 [SMILES: Brc1cc(-n2c3ccccc3c3cc(-c4ccccc4)ccc32)c2oc3ccccc3c2c1 ; "S194"] | 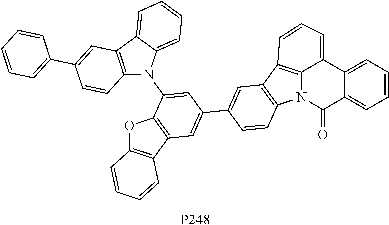 [SMILES: "P248" ; O=c1c2ccccc2c2cccc3c4cc(-c5cc(-n6c7ccccc7c7cc(-c8ccccc8)ccc76)c6oc7ccccc7c6c5)ccc4n1c23] |
| S125 | 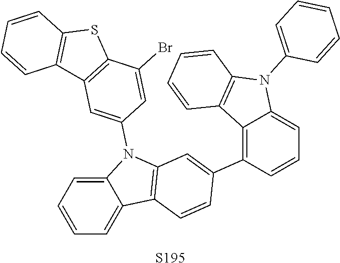 [SMILES: Brc1cc(-n2c3ccccc3c3ccc(-c4cccc5c4c4ccccc4n5-c4ccccc4)cc32)cc2c1sc1ccccc12 ; "S195"] | 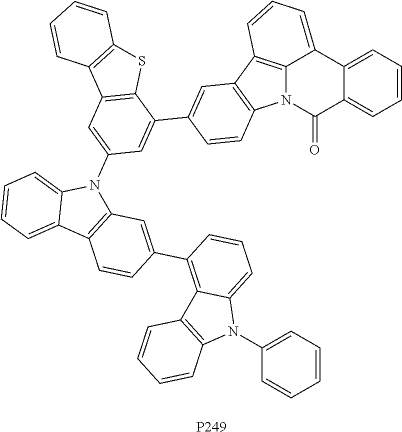 |
| S125 | 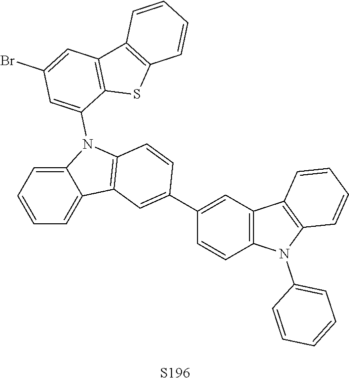 [SMILES: Brc1cc(-n2c3ccccc3c3cc(-c4ccc5c(c4)c4ccccc4n5-c4ccccc4)ccc32)c2sc3ccccc3c2c1 ; "S196"] | 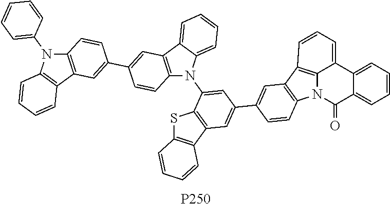 |
| S126 | 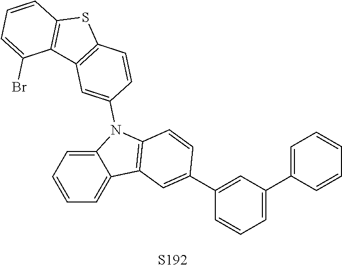 [SMILES: Brc1cccc2sc3ccc(-n4c5ccccc5c5cc(-c6cccc(-c7ccccc7)c6)ccc54)cc3c12 ; "S192"] | 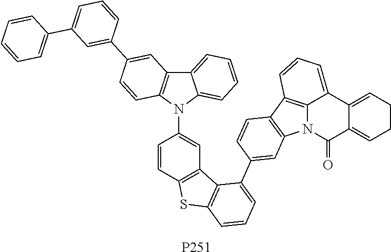 [SMILES: O=c1c2c(c3cccc4c5ccc(-c6cccc7sc8ccc(-n9c%10ccccc%10c%10cc(-c%11cccc(-c%12ccccc%12)c%11)ccc%109)cc8c67)cc5n1c34)=CCCC=2 ; "P251"] |
| S127 | 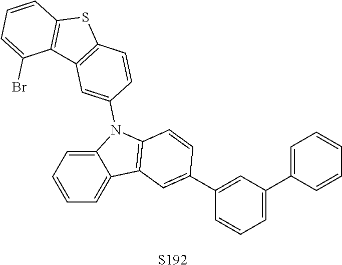 [SMILES: Brc1cccc2sc3ccc(-n4c5ccccc5c5cc(-c6cccc(-c7ccccc7)c6)ccc54)cc3c12 ; "S192"] | 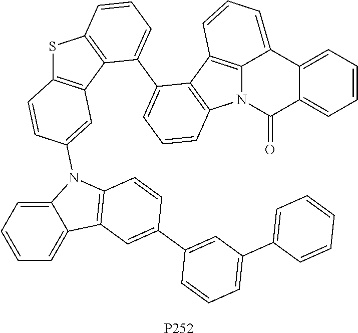 [SMILES: "P252" ; O=c1c2ccccc2c2cccc3c4c(-c5cccc6sc7ccc(-n8c9ccccc9c9cc(-c%10cccc(-c%11ccccc%11)c%10)ccc98)cc7c56)cccc4n1c23] |
| S128 | 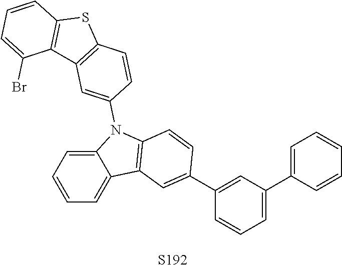 [SMILES: Brc1cccc2sc3ccc(-n4c5ccccc5c5cc(-c6cccc(-c7ccccc7)c6)ccc54)cc3c12 ; "S192"] | 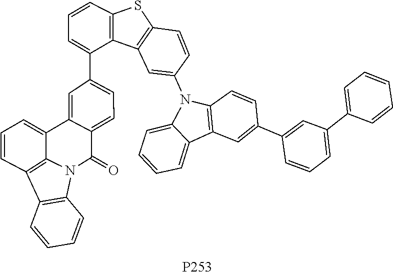 |
| S129 | 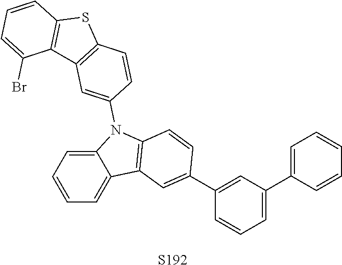 [SMILES: Brc1cccc2sc3ccc(-n4c5ccccc5c5cc(-c6cccc(-c7ccccc7)c6)ccc54)cc3c12 ; "S192"] | 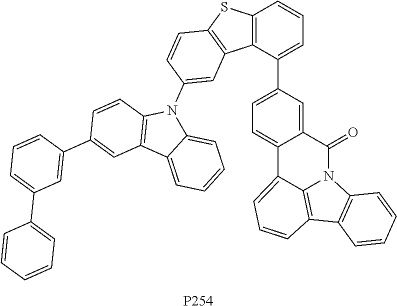 [SMILES: "P254" ; O=c1c2cc(-c3cccc4sc5ccc(-n6c7ccccc7c7cc(-c8cccc(-c9ccccc9)c8)ccc76)cc5c34)ccc2c2cccc3c4ccccc4n1c23] |
| S130 | 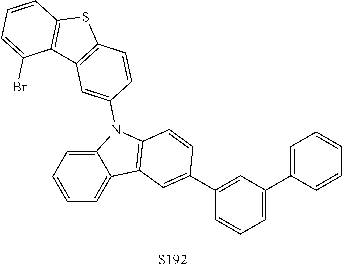 [SMILES: Brc1cccc2sc3ccc(-n4c5ccccc5c5cc(-c6cccc(-c7ccccc7)c6)ccc54)cc3c12 ; "S192"] | 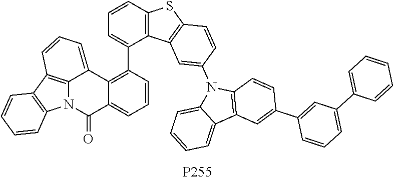 [SMILES: O=c1c2cccc(-c3cccc4sc5ccc(-n6c7ccccc7c7cc(-c8cccc(-c9ccccc9)c8)ccc76)cc5c34)c2c2cccc3c4ccccc4n1c32 ; "P255"] |
| S131 | 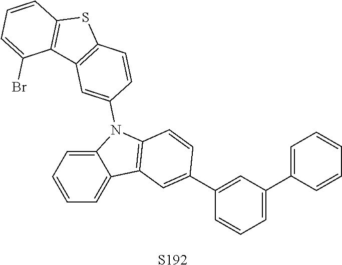 [SMILES: Brc1cccc2sc3ccc(-n4c5ccccc5c5cc(-c6cccc(-c7ccccc7)c6)ccc54)cc3c12 ; "S192"] | 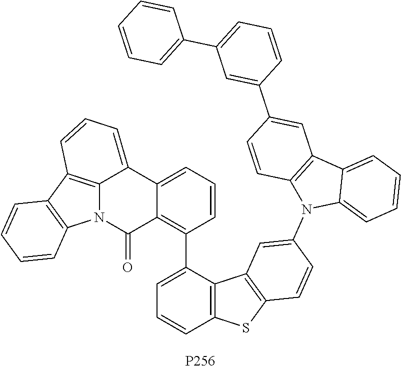 |
| S132 | 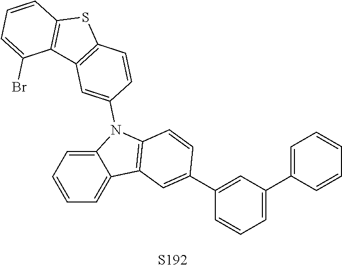 [SMILES: Brc1cccc2sc3ccc(-n4c5ccccc5c5cc(-c6cccc(-c7ccccc7)c6)ccc54)cc3c12 ; "S192"] | 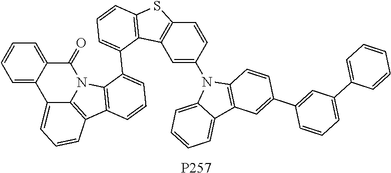 |
| S133 | 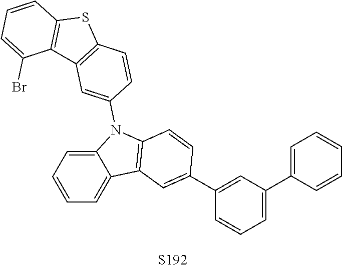 [SMILES: Brc1cccc2sc3ccc(-n4c5ccccc5c5cc(-c6cccc(-c7ccccc7)c6)ccc54)cc3c12 ; "S192"] | 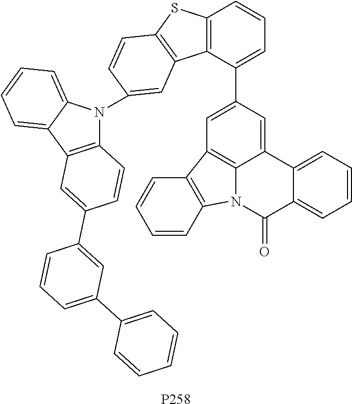 |
| S134 | 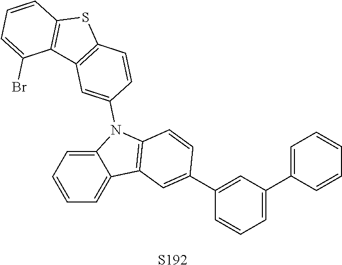 [SMILES: Brc1cccc2sc3ccc(-n4c5ccccc5c5cc(-c6cccc(-c7ccccc7)c6)ccc54)cc3c12 ; "S192"] | 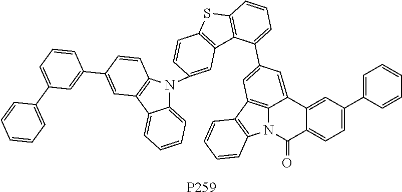 [SMILES: O=c1c2ccc(-c3ccccc3)cc2c2cc(-c3cccc4sc5ccc(-n6c7ccccc7c7cc(-c8cccc(-c9ccccc9)c8)ccc76)cc5c34)cc3c4ccccc4n1c23 ; "P259"] |
| S135 | 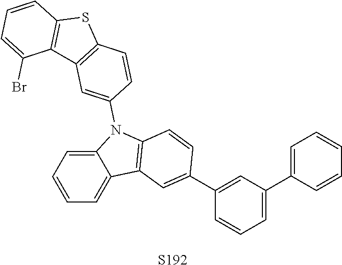 [SMILES: Brc1cccc2sc3ccc(-n4c5ccccc5c5cc(-c6cccc(-c7ccccc7)c6)ccc54)cc3c12 ; "S192"] | 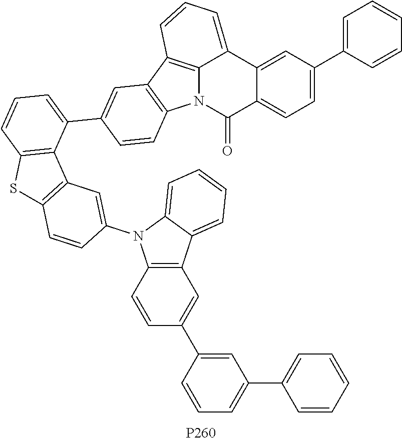 |
| S136 | 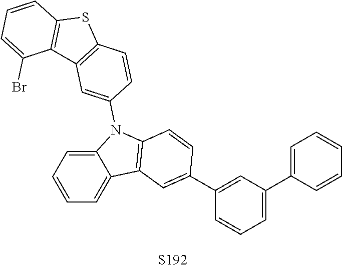 [SMILES: Brc1cccc2sc3ccc(-n4c5ccccc5c5cc(-c6cccc(-c7ccccc7)c6)ccc54)cc3c12 ; "S192"] | 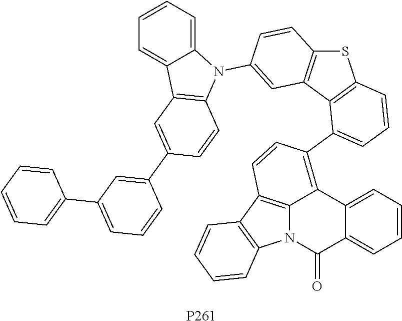 |
| S137 | 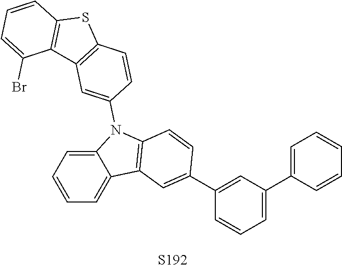 [SMILES: Brc1cccc2sc3ccc(-n4c5ccccc5c5cc(-c6cccc(-c7ccccc7)c6)ccc54)cc3c12 ; "S192"] | 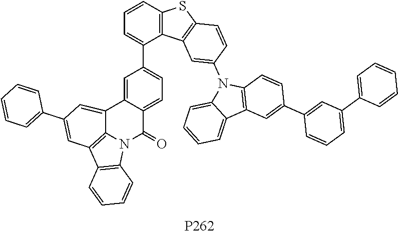 [SMILES: O=c1c2ccc(-c3cccc4sc5ccc(-n6c7ccccc7c7cc(-c8cccc(-c9ccccc9)c8)ccc76)cc5c34)cc2c2cc(-c3ccccc3)cc3c4ccccc4n1c23 ; "P262"] |
| S138 | 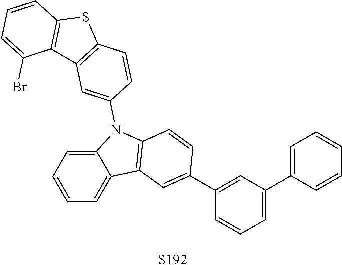 [SMILES: Brc1cccc2sc3ccc(-n4c5ccccc5c5cc(-c6cccc(-c7ccccc7)c6)ccc54)cc3c12 ; "S192"] | 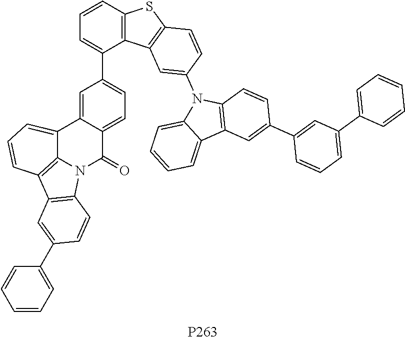 [SMILES: O=c1c2ccc(-c3cccc4sc5ccc(-n6c7ccccc7c7cc(-c8cccc(-c9ccccc9)c8)ccc76)cc5c34)cc2c2cccc3c4cc(-c5ccccc5)ccc4n1c23 ; "P263"] |
| S139 | 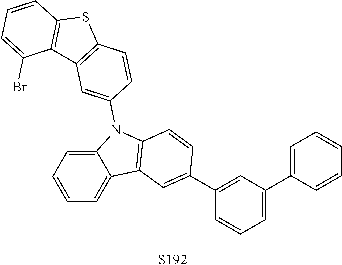 [SMILES: Brc1cccc2sc3ccc(-n4c5ccccc5c5cc(-c6cccc(-c7ccccc7)c6)ccc54)cc3c12 ; "S192"] | 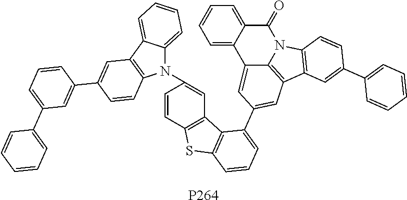 [SMILES: O=c1c2ccccc2c2cc(-c3cccc4sc5ccc(-n6c7ccccc7c7cc(-c8cccc(-c9ccccc9)c8)ccc76)cc5c34)cc3c4cc(-c5ccccc5)ccc4n1c23 ; "P264"] |
| S140 | 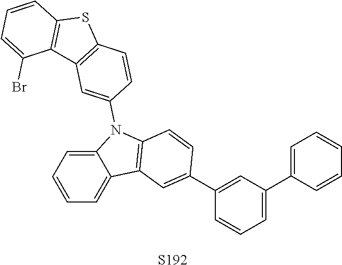 [SMILES: Brc1cccc2sc3ccc(-n4c5ccccc5c5cc(-c6cccc(-c7ccccc7)c6)ccc54)cc3c12 ; "S192"] | 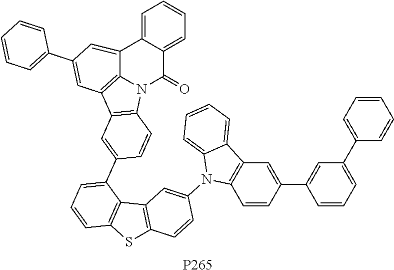 |
| S126 | 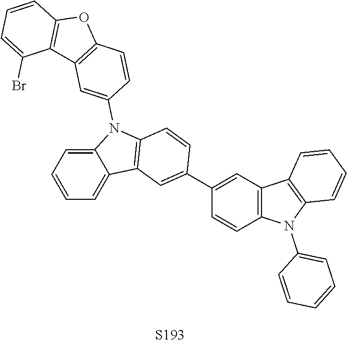 [SMILES: Brc1cccc2oc3ccc(-n4c5ccccc5c5cc(-c6ccc7c(c6)c6ccccc6n7-c6ccccc6)ccc54)cc3c12 ; "S193"] | 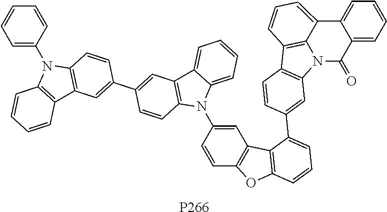 [SMILES: O=c1c2ccccc2c2cccc3c4ccc(-c5cccc6oc7ccc(-n8c9ccccc9c9cc(-c%10ccc%11c(c%10)c%10ccccc%10n%11-c%10ccccc%10)ccc98)cc7c56)cc4n1c23 ; "P266"] |
| S127 | 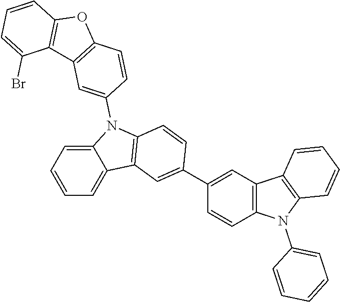 [SMILES: Brc1cccc2oc3ccc(-n4c5ccccc5c5cc(-c6ccc7c(c6)c6ccccc6n7-c6ccccc6)ccc54)cc3c12] | 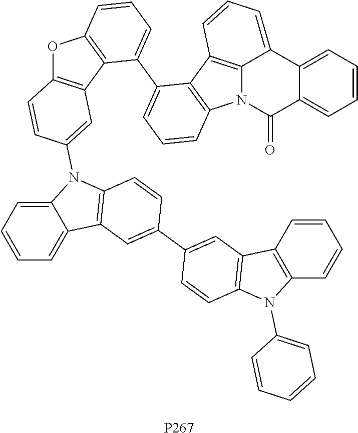 [SMILES: O=c1c2ccccc2c2cccc3c4c(-c5cccc6oc7ccc(-n8c9ccccc9c9cc(-c%10ccc%11c(c%10)c%10ccccc%10n%11-c%10ccccc%10)ccc98)cc7c56)cccc4n1c23 ; "P267"] |
| S128 | 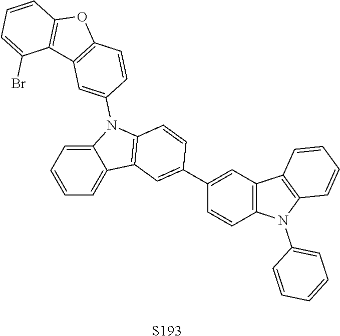 [SMILES: Brc1cccc2oc3ccc(-n4c5ccccc5c5cc(-c6ccc7c(c6)c6ccccc6n7-c6ccccc6)ccc54)cc3c12 ; "S193"] | 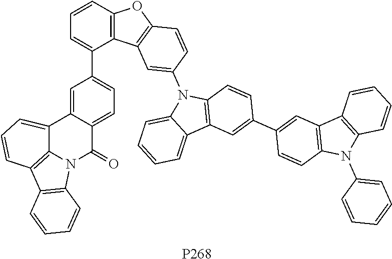 [SMILES: O=c1c2ccc(-c3cccc4oc5ccc(-n6c7ccccc7c7cc(-c8ccc9c(c8)c8ccccc8n9-c8ccccc8)ccc76)cc5c34)cc2c2cccc3c4ccccc4n1c23 ; "P268"] |
| S129 | 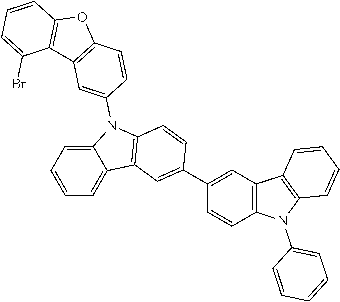 [SMILES: Brc1cccc2oc3ccc(-n4c5ccccc5c5cc(-c6ccc7c(c6)c6ccccc6n7-c6ccccc6)ccc54)cc3c12] | 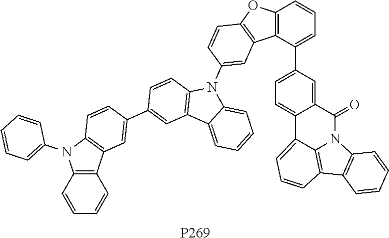 [SMILES: O=c1c2cc(-c3cccc4oc5ccc(-n6c7ccccc7c7cc(-c8ccc9c(c8)c8ccccc8n9-c8ccccc8)ccc76)cc5c34)ccc2c2cccc3c4ccccc4n1c23 ; "P269"] |
| S130 | 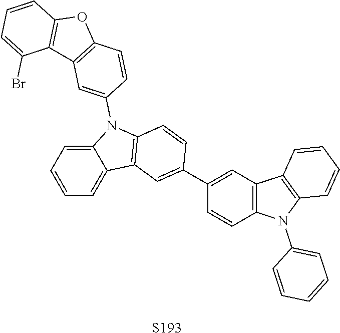 [SMILES: Brc1cccc2oc3ccc(-n4c5ccccc5c5cc(-c6ccc7c(c6)c6ccccc6n7-c6ccccc6)ccc54)cc3c12 ; "S193"] | 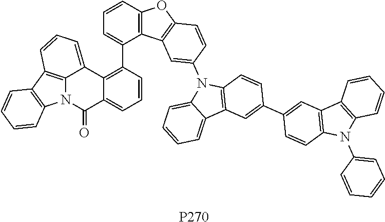 [SMILES: O=c1c2cccc(-c3cccc4oc5ccc(-n6c7ccccc7c7cc(-c8ccc9c(c8)c8ccccc8n9-c8ccccc8)ccc76)cc5c34)c2c2cccc3c4ccccc4n1c32 ; "P270"] |
| S131 | 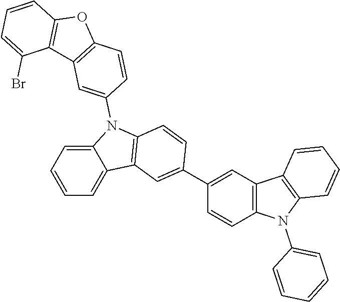 [SMILES: Brc1cccc2oc3ccc(-n4c5ccccc5c5cc(-c6ccc7c(c6)c6ccccc6n7-c6ccccc6)ccc54)cc3c12] | 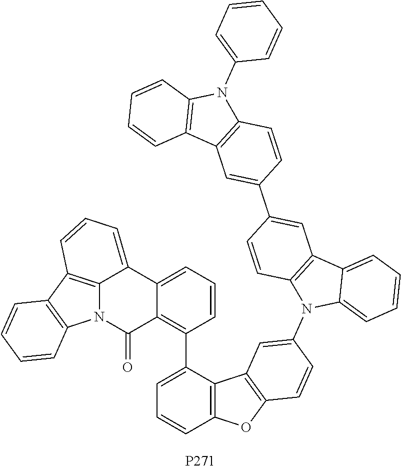 |
| S132 | 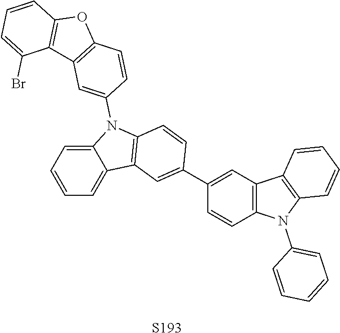 [SMILES: Brc1cccc2oc3ccc(-n4c5ccccc5c5cc(-c6ccc7c(c6)c6ccccc6n7-c6ccccc6)ccc54)cc3c12 ; "S193"] | 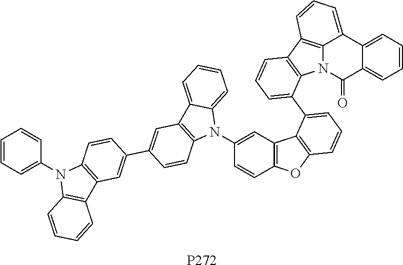 [SMILES: O=c1c2ccccc2c2cccc3c4cccc(-c5cccc6oc7ccc(-n8c9ccccc9c9cc(-c%10ccc%11c(c%10)c%10ccccc%10n%11-c%10ccccc%10)ccc98)cc7c56)c4n1c23 ; "P272"] |
| S133 | 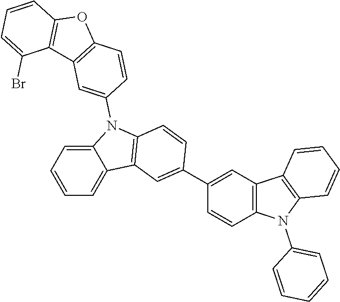 [SMILES: Brc1cccc2oc3ccc(-n4c5ccccc5c5cc(-c6ccc7c(c6)c6ccccc6n7-c6ccccc6)ccc54)cc3c12] | 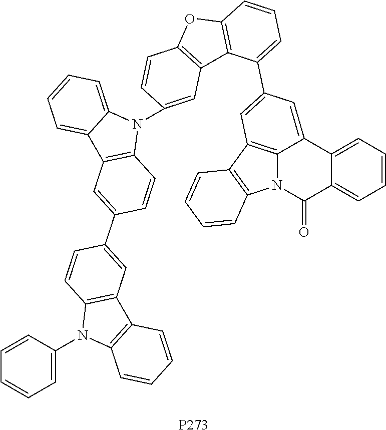 [SMILES: O=c1c2ccccc2c2cc(-c3cccc4oc5ccc(-n6c7ccccc7c7cc(-c8ccc9c(c8)c8ccccc8n9-c8ccccc8)ccc76)cc5c34)cc3c4ccccc4n1c23 ; "P273"] |
| S134 | 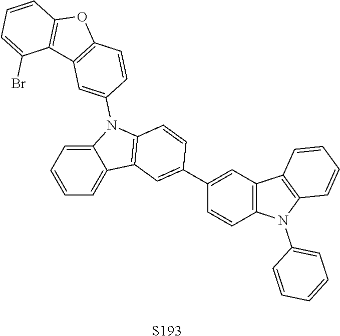 [SMILES: Brc1cccc2oc3ccc(-n4c5ccccc5c5cc(-c6ccc7c(c6)c6ccccc6n7-c6ccccc6)ccc54)cc3c12 ; "S193"] | 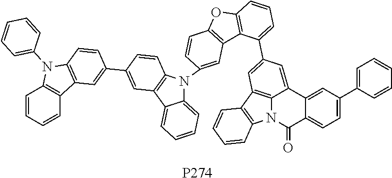 [SMILES: O=c1c2ccc(-c3ccccc3)cc2c2cc(-c3cccc4oc5ccc(-n6c7ccccc7c7cc(-c8ccc9c(c8)c8ccccc8n9-c8ccccc8)ccc76)cc5c34)cc3c4ccccc4n1c23 ; "P274"] |
| S135 | 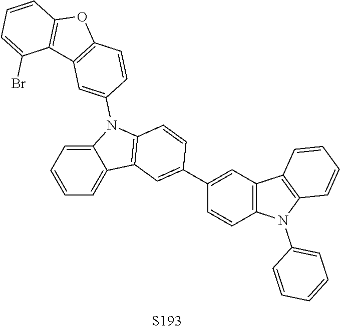 [SMILES: Brc1cccc2oc3ccc(-n4c5ccccc5c5cc(-c6ccc7c(c6)c6ccccc6n7-c6ccccc6)ccc54)cc3c12 ; "S193"] | 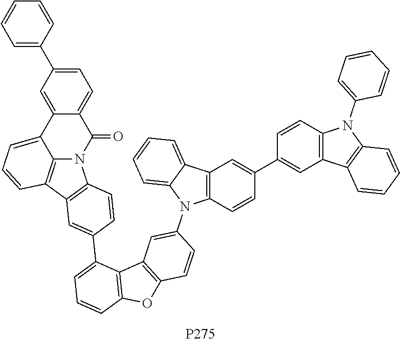 [SMILES: O=c1c2ccc(-c3ccccc3)cc2c2cccc3c4cc(-c5cccc6oc7ccc(-n8c9ccccc9c9cc(-c%10ccc%11c(c%10)c%10ccccc%10n%11-c%10ccccc%10)ccc98)cc7c56)ccc4n1c23 ; "P275"] |
| S136 | 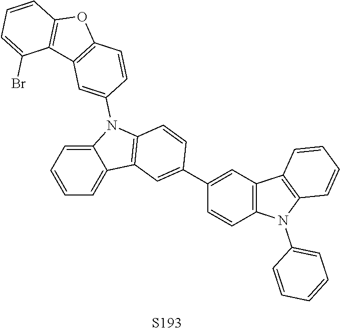 [SMILES: Brc1cccc2oc3ccc(-n4c5ccccc5c5cc(-c6ccc7c(c6)c6ccccc6n7-c6ccccc6)ccc54)cc3c12 ; "S193"] | 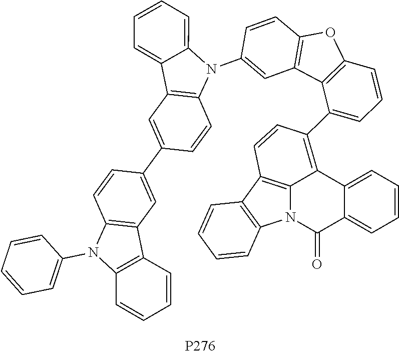 [SMILES: O=c1c2ccccc2c2c(-c3cccc4oc5ccc(-n6c7ccccc7c7cc(-c8ccc9c(c8)c8ccccc8n9-c8ccccc8)ccc76)cc5c34)ccc3c4ccccc4n1c32 ; "P276"] |
| S137 | 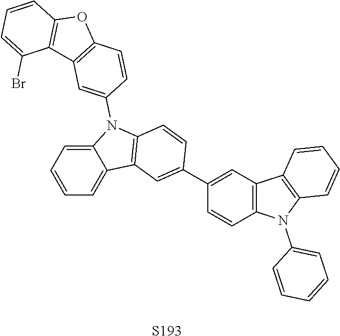 [SMILES: Brc1cccc2oc3ccc(-n4c5ccccc5c5cc(-c6ccc7c(c6)c6ccccc6n7-c6ccccc6)ccc54)cc3c12 ; "S193"] | 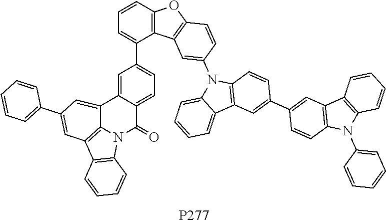 [SMILES: O=c1c2ccc(-c3cccc4oc5ccc(-n6c7ccccc7c7cc(-c8ccc9c(c8)c8ccccc8n9-c8ccccc8)ccc76)cc5c34)cc2c2cc(-c3ccccc3)cc3c4ccccc4n1c23 ; "P277"] |
| S138 | 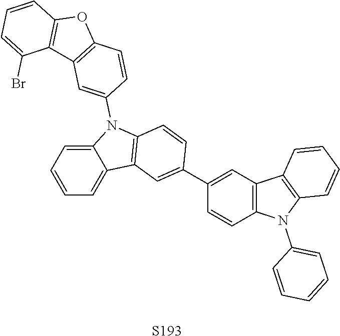 [SMILES: Brc1cccc2oc3ccc(-n4c5ccccc5c5cc(-c6ccc7c(c6)c6ccccc6n7-c6ccccc6)ccc54)cc3c12 ; "S193"] | 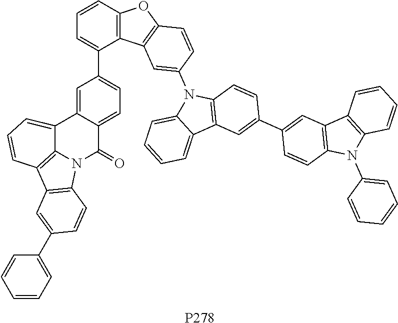 [SMILES: O=c1c2ccc(-c3cccc4oc5ccc(-n6c7ccccc7c7cc(-c8ccc9c(c8)c8ccccc8n9-c8ccccc8)ccc76)cc5c34)cc2c2cccc3c4cc(-c5ccccc5)ccc4n1c23 ; "P278"] |
| S139 | 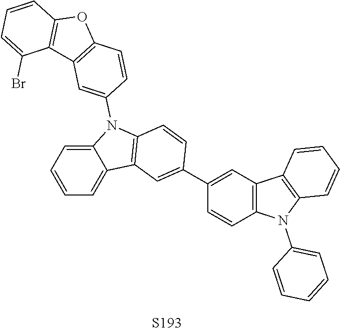 [SMILES: Brc1cccc2oc3ccc(-n4c5ccccc5c5cc(-c6ccc7c(c6)c6ccccc6n7-c6ccccc6)ccc54)cc3c12 ; "S193"] | 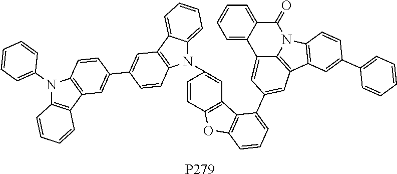 [SMILES: O=c1c2ccccc2c2cc(-c3cccc4oc5ccc(-n6c7ccccc7c7cc(-c8ccc9c(c8)c8ccccc8n9-c8ccccc8)ccc76)cc5c34)cc3c4cc(-c5ccccc5)ccc4n1c23 ; "P279"] |
| S140 | 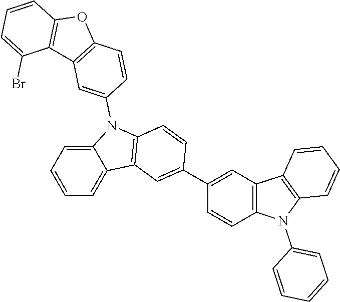 [SMILES: Brc1cccc2oc3ccc(-n4c5ccccc5c5cc(-c6ccc7c(c6)c6ccccc6n7-c6ccccc6)ccc54)cc3c12] | 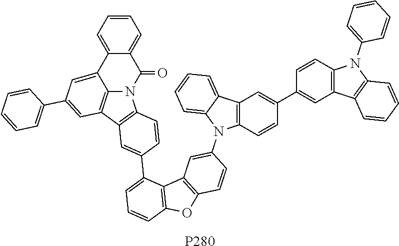 |
| S128 | 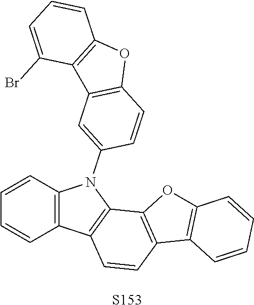 [SMILES: Brc1cccc2oc3ccc(-n4c5ccccc5c5ccc6c7ccccc7oc6c54)cc3c12 ; "S153"] | 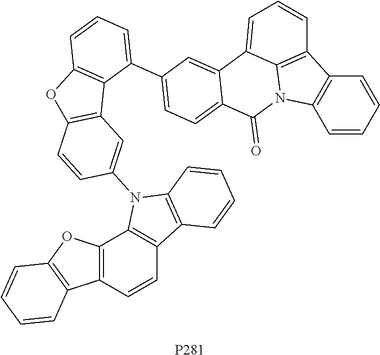 [SMILES: O=c1c2ccc(-c3cccc4oc5ccc(-n6c7ccccc7c7ccc8c9ccccc9oc8c76)cc5c34)cc2c2cccc3c4ccccc4n1c23 ; "P281"] |
| S128 | 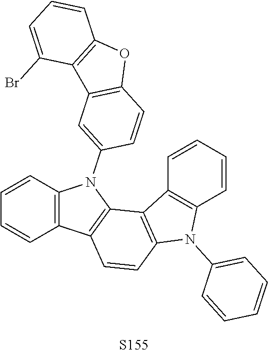 [SMILES: Brc1cccc2oc3ccc(-n4c5ccccc5c5ccc6c(c7ccccc7n6-c6ccccc6)c54)cc3c12 ; "S155"] | 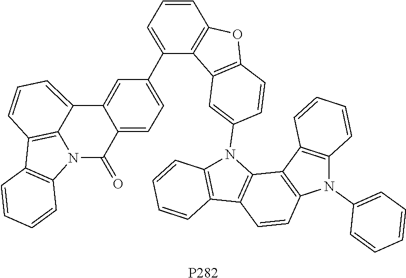 [SMILES: O=c1c2ccc(-c3cccc4oc5ccc(-n6c7ccccc7c7ccc8c(c9ccccc9n8-c8ccccc8)c76)cc5c34)cc2c2cccc3c4ccccc4n1c23 ; "P282"] |
| S128 | 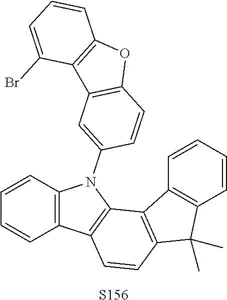 [SMILES: CC1(C)c2ccccc2-c2c1ccc1c3ccccc3n(-c3ccc4oc5cccc(Br)c5c4c3)c21 ; "S156"] | 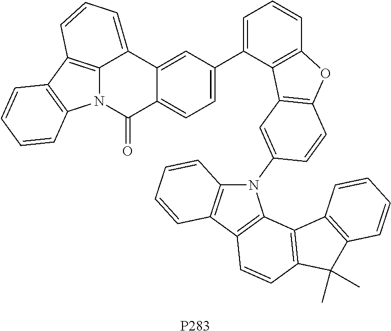 [SMILES: CC1(C)c2ccccc2-c2c1ccc1c3ccccc3n(-c3ccc4oc5cccc(-c6ccc7c(=O)n8c9ccccc9c9cccc(c7c6)c98)c5c4c3)c21 ; "P283"] |
| S128 | 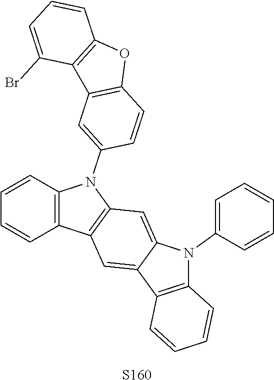 [SMILES: Brc1cccc2oc3ccc(-n4c5ccccc5c5cc6c7ccccc7n(-c7ccccc7)c6cc54)cc3c12 ; "S160"] | 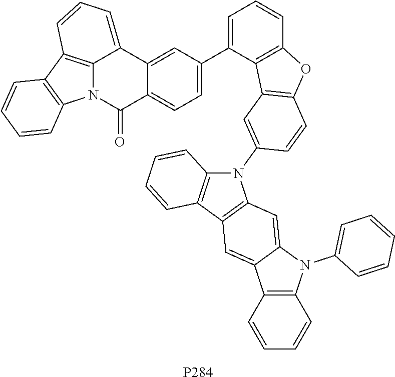 [SMILES: O=c1c2ccc(-c3cccc4oc5ccc(-n6c7ccccc7c7cc8c9ccccc9n(-c9ccccc9)c8cc76)cc5c34)cc2c2cccc3c4ccccc4n1c23 ; "P284"] |
| S128 | 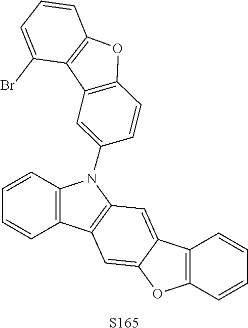 [SMILES: Brc1cccc2oc3ccc(-n4c5ccccc5c5cc6oc7ccccc7c6cc54)cc3c12 ; "S165"] | 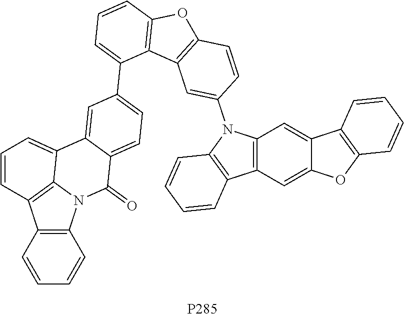 [SMILES: O=c1c2ccc(-c3cccc4oc5ccc(-n6c7ccccc7c7cc8oc9ccccc9c8cc76)cc5c34)cc2c2cccc3c4ccccc4n1c23 ; "P285"] |
| S128 | 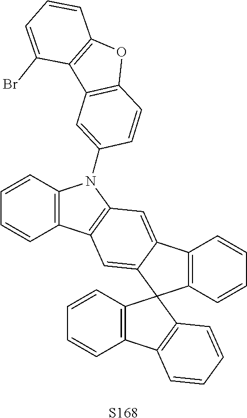 [SMILES: Brc1cccc2oc3ccc(-n4c5ccccc5c5cc6c(cc54)-c4ccccc4C64c5ccccc5-c5ccccc54)cc3c12 ; "S168"] | 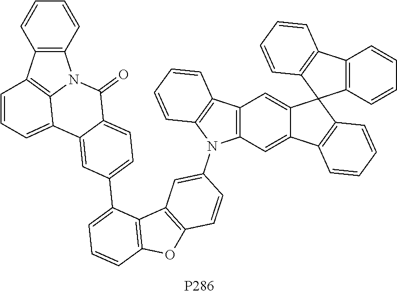 |
| S128 | 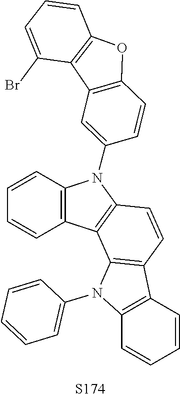 [SMILES: Brc1cccc2oc3ccc(-n4c5ccccc5c5c4ccc4c6ccccc6n(-c6ccccc6)c45)cc3c12 ; "S174"] | 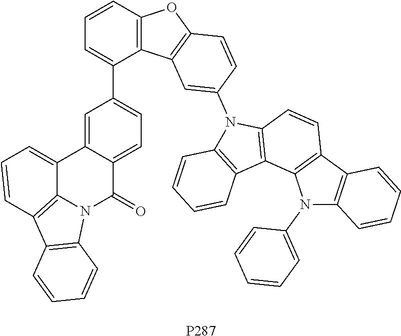 [SMILES: O=c1c2ccc(-c3cccc4oc5ccc(-n6c7ccccc7c7c6ccc6c8ccccc8n(-c8ccccc8)c67)cc5c34)cc2c2cccc3c4ccccc4n1c23 ; "P287"] |
| S128 | 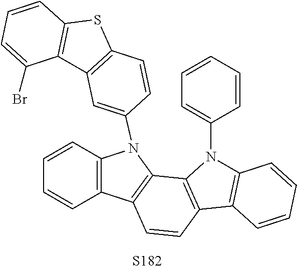 [SMILES: Brc1cccc2sc3ccc(-n4c5ccccc5c5ccc6c7ccccc7n(-c7ccccc7)c6c54)cc3c12 ; "S182"] | 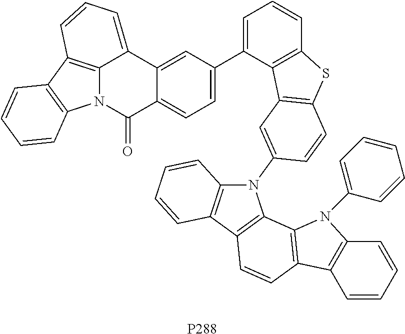 [SMILES: O=c1c2ccc(-c3cccc4sc5ccc(-n6c7ccccc7c7ccc8c9ccccc9n(-c9ccccc9)c8c76)cc5c34)cc2c2cccc3c4ccccc4n1c23 ; "P288"] |
| S128 | 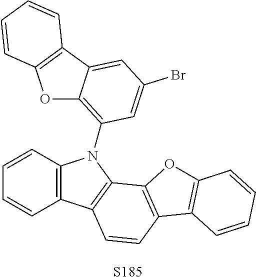 [SMILES: Brc1cc(-n2c3ccccc3c3ccc4c5ccccc5oc4c32)c2oc3ccccc3c2c1 ; "S185"] | 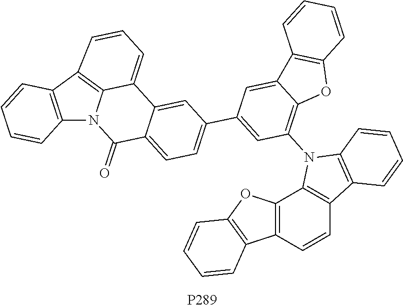 |
| S128 | 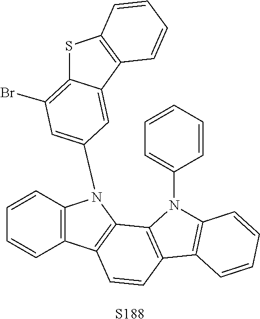 [SMILES: Brc1cc(-n2c3ccccc3c3ccc4c5ccccc5n(-c5ccccc5)c4c32)cc2c1sc1ccccc12 ; "S188"] | 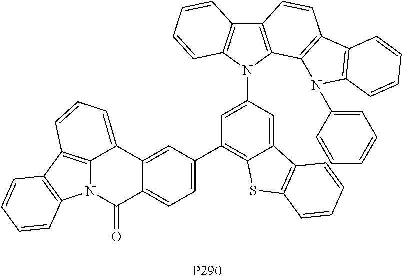 [SMILES: O=c1c2ccc(-c3cc(-n4c5ccccc5c5ccc6c7ccccc7n(-c7ccccc7)c6c54)cc4c3sc3ccccc34)cc2c2cccc3c4ccccc4n1c23 ; "P290"] |
| S128 | 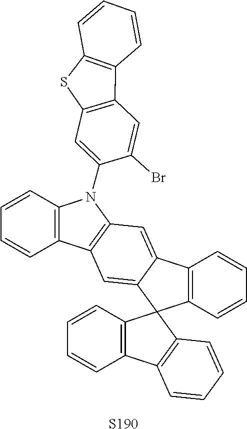 [SMILES: Brc1cc2c(cc1-n1c3ccccc3c3cc4c(cc31)-c1ccccc1C41c3ccccc3-c3ccccc31)sc1ccccc12 ; "S190"] | 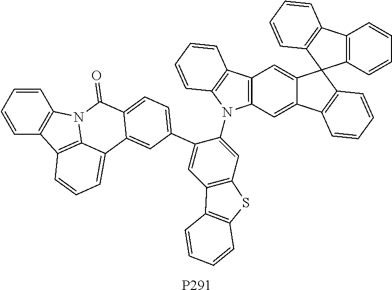 |
| S128 | 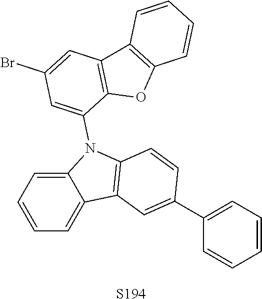 [SMILES: Brc1cc(-n2c3ccccc3c3cc(-c4ccccc4)ccc32)c2oc3ccccc3c2c1 ; "S194"] | 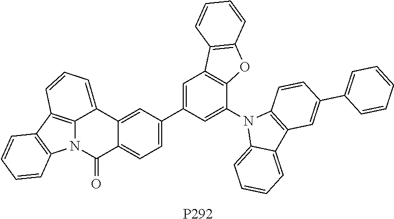 [SMILES: O=c1c2ccc(-c3cc(-n4c5ccccc5c5cc(-c6ccccc6)ccc54)c4oc5ccccc5c4c3)cc2c2cccc3c4ccccc4n1c23 ; "P292"] |
| S133 | 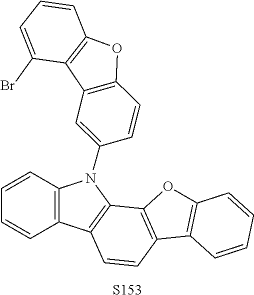 [SMILES: Brc1cccc2oc3ccc(-n4c5ccccc5c5ccc6c7ccccc7oc6c54)cc3c12 ; "S153"] | 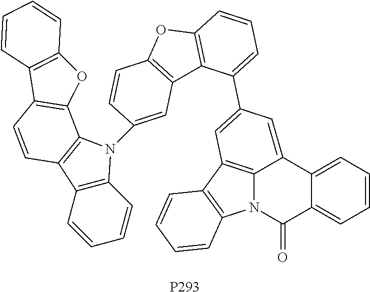 |
| S133 | 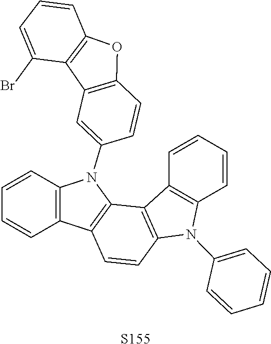 [SMILES: Brc1cccc2oc3ccc(-n4c5ccccc5c5ccc6c(c7ccccc7n6-c6ccccc6)c54)cc3c12 ; "S155"] | 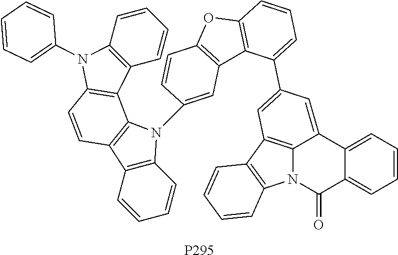 |
| S133 | 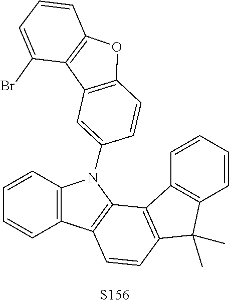 [SMILES: CC1(C)c2ccccc2-c2c1ccc1c3ccccc3n(-c3ccc4oc5cccc(Br)c5c4c3)c21 ; "S156"] | 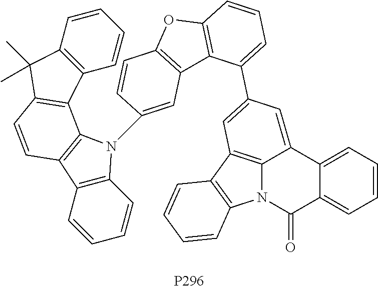 |
| S133 | 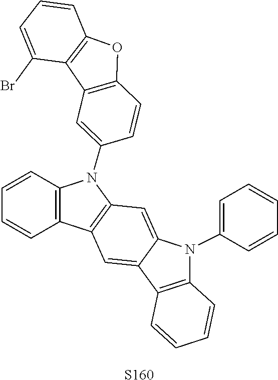 [SMILES: Brc1cccc2oc3ccc(-n4c5ccccc5c5cc6c7ccccc7n(-c7ccccc7)c6cc54)cc3c12 ; "S160"] | 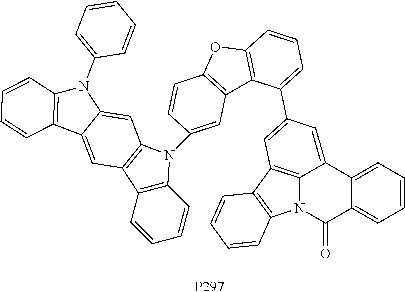 [SMILES: O=c1c2ccccc2c2cc(-c3cccc4oc5ccc(-n6c7ccccc7c7cc8c9ccccc9n(-c9ccccc9)c8cc76)cc5c34)cc3c4ccccc4n1c23 ; "P297"] |
| S133 | 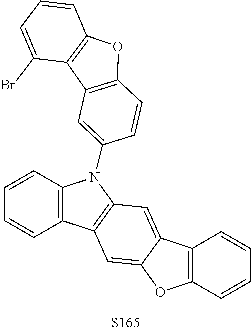 [SMILES: Brc1cccc2oc3ccc(-n4c5ccccc5c5cc6oc7ccccc7c6cc54)cc3c12 ; "S165"] | 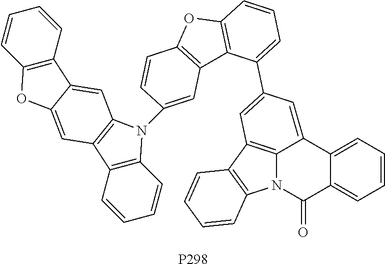 |
| S133 | 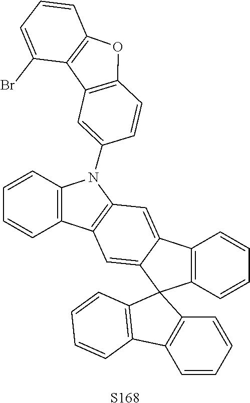 [SMILES: Brc1cccc2oc3ccc(-n4c5ccccc5c5cc6c(cc54)-c4ccccc4C64c5ccccc5-c5ccccc54)cc3c12 ; "S168"] | 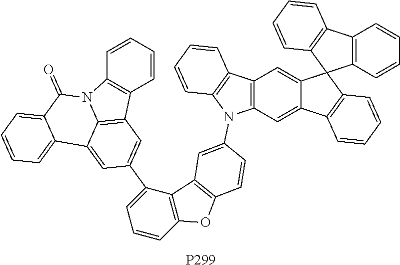 |
| S133 | 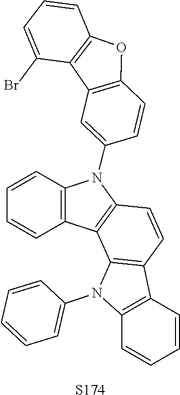 [SMILES: Brc1cccc2oc3ccc(-n4c5ccccc5c5c4ccc4c6ccccc6n(-c6ccccc6)c45)cc3c12 ; "S174"] | 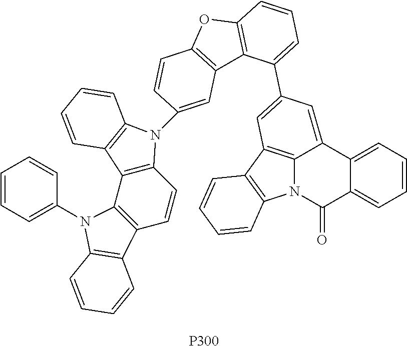 [SMILES: O=c1c2ccccc2c2cc(-c3cccc4oc5ccc(-n6c7ccccc7c7c6ccc6c8ccccc8n(-c8ccccc8)c67)cc5c34)cc3c4ccccc4n1c23 ; "P300"] |
| S133 | 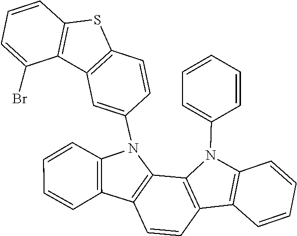 [SMILES: Brc1cccc2sc3ccc(-n4c5ccccc5c5ccc6c7ccccc7n(-c7ccccc7)c6c54)cc3c12] | 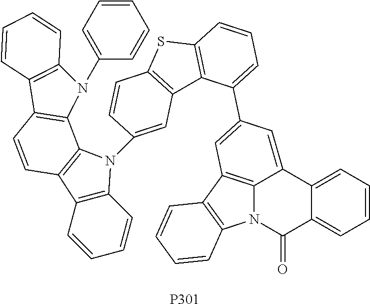 |
| S133 | 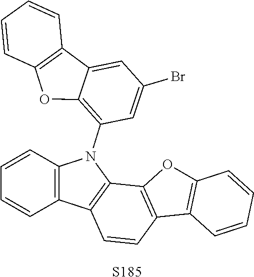 [SMILES: Brc1cc(-n2c3ccccc3c3ccc4c5ccccc5oc4c32)c2oc3ccccc3c2c1 ; "S185"] | 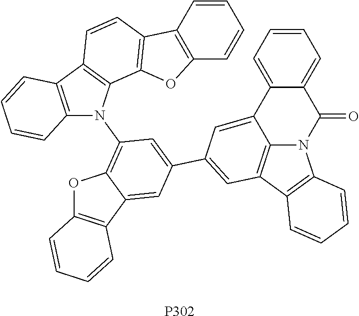 [SMILES: O=c1c2ccccc2c2cc(-c3cc(-n4c5ccccc5c5ccc6c7ccccc7oc6c54)c4oc5ccccc5c4c3)cc3c4ccccc4n1c23 ; "P302"] |
| S133 | 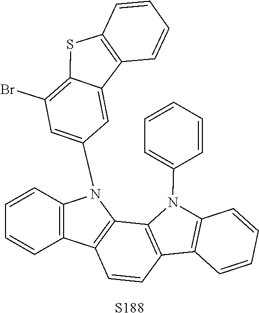 [SMILES: Brc1cc(-n2c3ccccc3c3ccc4c5ccccc5n(-c5ccccc5)c4c32)cc2c1sc1ccccc12 ; "S188"] | 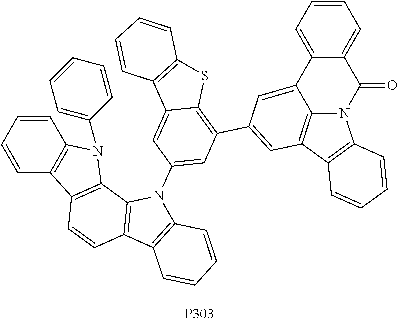 |
| S133 | 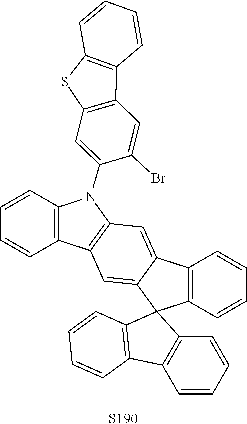 [SMILES: Brc1cc2c(cc1-n1c3ccccc3c3cc4c(cc31)-c1ccccc1C41c3ccccc3-c3ccccc31)sc1ccccc12 ; "S190"] | 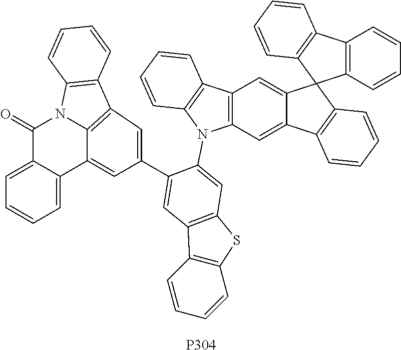 |
| S133 | 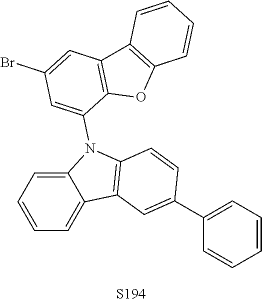 [SMILES: Brc1cc(-n2c3ccccc3c3cc(-c4ccccc4)ccc32)c2oc3ccccc3c2c1 ; "S194"] | 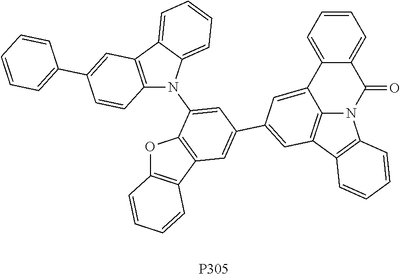 [SMILES: O=c1c2ccccc2c2cc(-c3cc(-n4c5ccccc5c5cc(-c6ccccc6)ccc54)c4oc5ccccc5c4c3)cc3c4ccccc4n1c23 ; "P305"] |
| S135 | 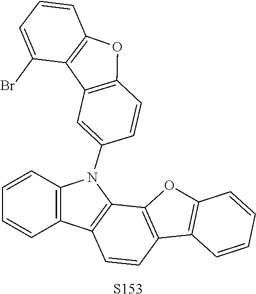 [SMILES: Brc1cccc2oc3ccc(-n4c5ccccc5c5ccc6c7ccccc7oc6c54)cc3c12 ; "S153"] | 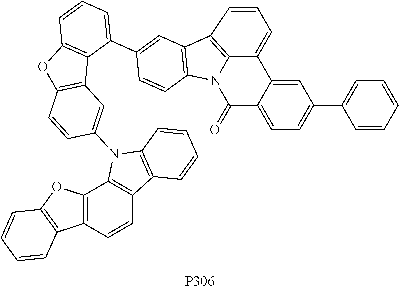 [SMILES: O=c1c2ccc(-c3ccccc3)cc2c2cccc3c4cc(-c5cccc6oc7ccc(-n8c9ccccc9c9ccc%10c%11ccccc%11oc%10c98)cc7c56)ccc4n1c23 ; "P306"] |
| S135 | 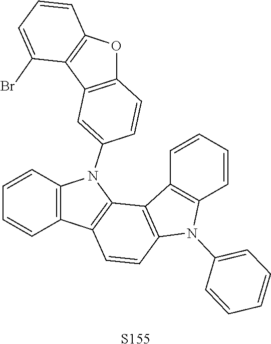 [SMILES: Brc1cccc2oc3ccc(-n4c5ccccc5c5ccc6c(c7ccccc7n6-c6ccccc6)c54)cc3c12 ; "S155"] | 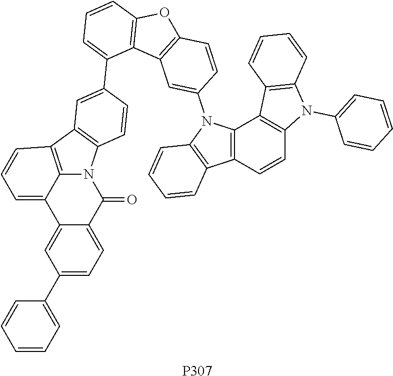 [SMILES: O=c1c2ccc(-c3ccccc3)cc2c2cccc3c4cc(-c5cccc6oc7ccc(-n8c9ccccc9c9ccc%10c(c%11ccccc%11n%10-c%10ccccc%10)c98)cc7c56)ccc4n1c23 ; "P307"] |
| S135 | 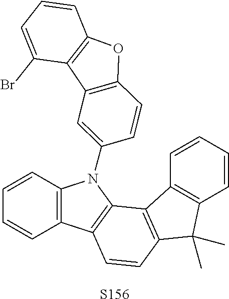 [SMILES: CC1(C)c2ccccc2-c2c1ccc1c3ccccc3n(-c3ccc4oc5cccc(Br)c5c4c3)c21 ; "S156"] | 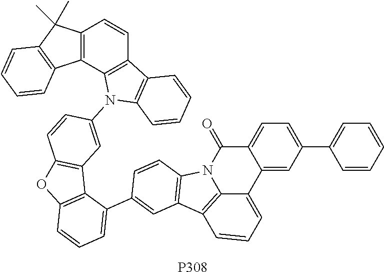 [SMILES: CC1(C)c2ccccc2-c2c1ccc1c3ccccc3n(-c3ccc4oc5cccc(-c6ccc7c(c6)c6cccc8c9cc(-c%10ccccc%10)ccc9c(=O)n7c86)c5c4c3)c21 ; "P308"] |
| S135 | 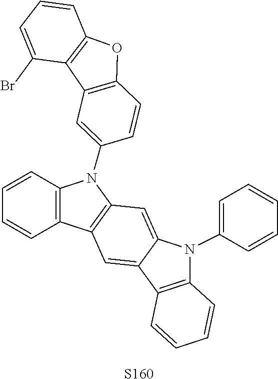 [SMILES: Brc1cccc2oc3ccc(-n4c5ccccc5c5cc6c7ccccc7n(-c7ccccc7)c6cc54)cc3c12 ; "S160"] | 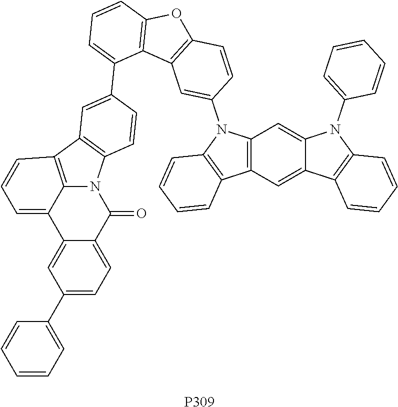 [SMILES: O=c1c2ccc(-c3ccccc3)cc2c2cccc3c4cc(-c5cccc6oc7ccc(-n8c9ccccc9c9cc%10c%11ccccc%11n(-c%11ccccc%11)c%10cc98)cc7c56)ccc4n1c23 ; "P309"] |
| S135 | 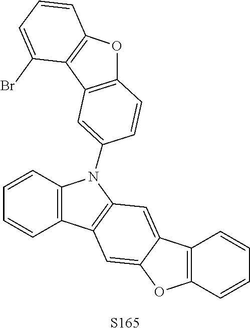 [SMILES: Brc1cccc2oc3ccc(-n4c5ccccc5c5cc6oc7ccccc7c6cc54)cc3c12 ; "S165"] | 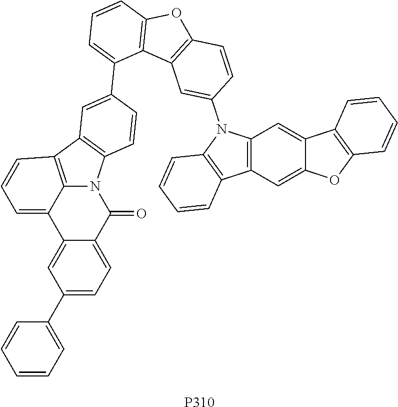 [SMILES: O=c1c2ccc(-c3ccccc3)cc2c2cccc3c4cc(-c5cccc6oc7ccc(-n8c9ccccc9c9cc%10oc%11ccccc%11c%10cc98)cc7c56)ccc4n1c23 ; "P310"] |
| S135 | 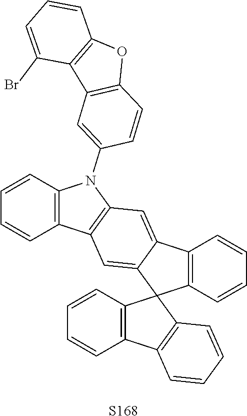 [SMILES: Brc1cccc2oc3ccc(-n4c5ccccc5c5cc6c(cc54)-c4ccccc4C64c5ccccc5-c5ccccc54)cc3c12 ; "S168"] | 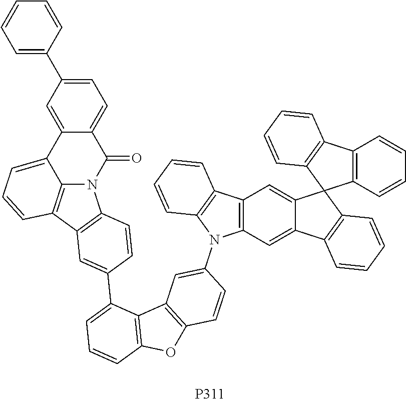 [SMILES: O=c1c2ccc(-c3ccccc3)cc2c2cccc3c4cc(-c5cccc6oc7ccc(-n8c9ccccc9c9cc%10c(cc98)-c8ccccc8C%108c9ccccc9-c9ccccc98)cc7c56)ccc4n1c23 ; "P311"] |
| S135 | 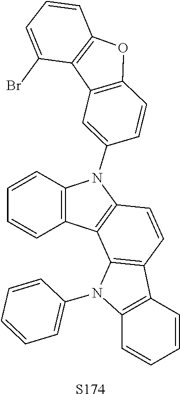 [SMILES: Brc1cccc2oc3ccc(-n4c5ccccc5c5c4ccc4c6ccccc6n(-c6ccccc6)c45)cc3c12 ; "S174"] | 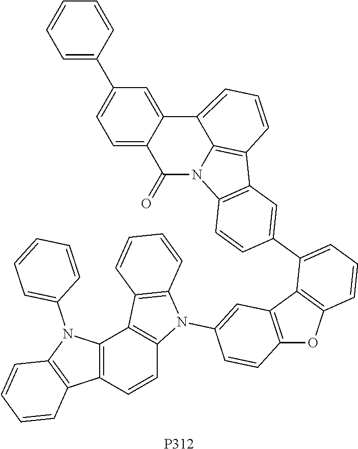 [SMILES: O=c1c2ccc(-c3ccccc3)cc2c2cccc3c4cc(-c5cccc6oc7ccc(-n8c9ccccc9c9c8ccc8c%10ccccc%10n(-c%10ccccc%10)c89)cc7c56)ccc4n1c23 ; "P312"] |
| S135 | 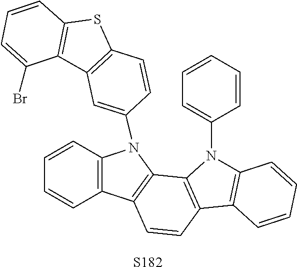 [SMILES: Brc1cccc2sc3ccc(-n4c5ccccc5c5ccc6c7ccccc7n(-c7ccccc7)c6c54)cc3c12 ; "S182"] | 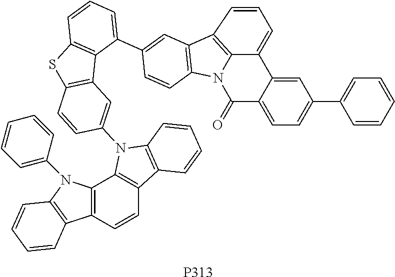 [SMILES: O=c1c2ccc(-c3ccccc3)cc2c2cccc3c4cc(-c5cccc6sc7ccc(-n8c9ccccc9c9ccc%10c%11ccccc%11n(-c%11ccccc%11)c%10c98)cc7c56)ccc4n1c23 ; "P313"] |
| S135 | 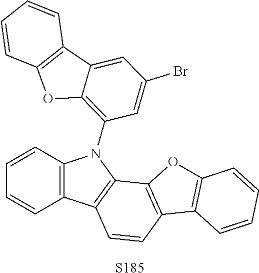 [SMILES: Brc1cc(-n2c3ccccc3c3ccc4c5ccccc5oc4c32)c2oc3ccccc3c2c1 ; "S185"] | 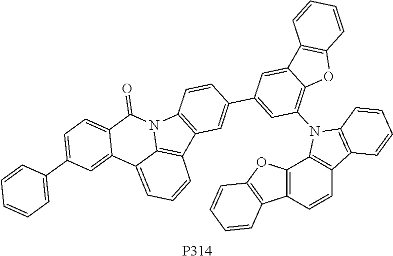 [SMILES: O=c1c2ccc(-c3ccccc3)cc2c2cccc3c4cc(-c5cc(-n6c7ccccc7c7ccc8c9ccccc9oc8c76)c6oc7ccccc7c6c5)ccc4n1c23 ; "P314"] |
| S135 | 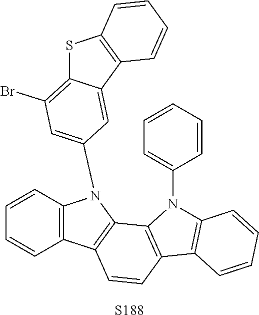 [SMILES: Brc1cc(-n2c3ccccc3c3ccc4c5ccccc5n(-c5ccccc5)c4c32)cc2c1sc1ccccc12 ; "S188"] | 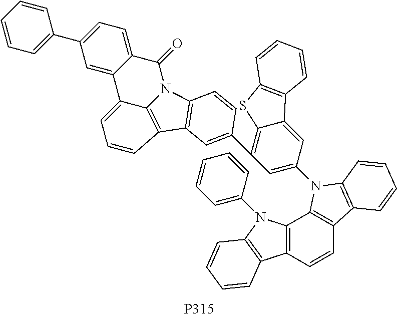 [SMILES: O=c1c2ccc(-c3ccccc3)cc2c2cccc3c4cc(-c5cc(-n6c7ccccc7c7ccc8c9ccccc9n(-c9ccccc9)c8c76)cc6c5sc5ccccc56)ccc4n1c23 ; "P315"] |
| S135 | 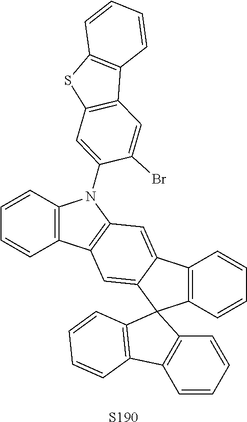 [SMILES: Brc1cc2c(cc1-n1c3ccccc3c3cc4c(cc31)-c1ccccc1C41c3ccccc3-c3ccccc31)sc1ccccc12 ; "S190"] | 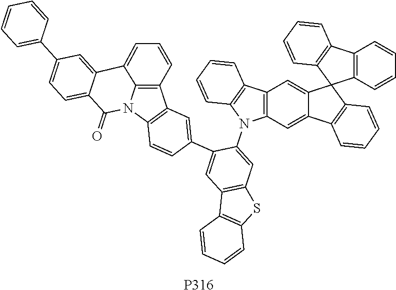 [SMILES: O=c1c2ccc(-c3ccccc3)cc2c2cccc3c4cc(-c5cc6c(cc5-n5c7ccccc7c7cc8c(cc75)-c5ccccc5C85c7ccccc7-c7ccccc75)sc5ccccc56)ccc4n1c23 ; "P316"] |
| S135 | 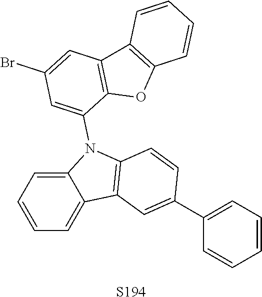 [SMILES: Brc1cc(-n2c3ccccc3c3cc(-c4ccccc4)ccc32)c2oc3ccccc3c2c1 ; "S194"] | 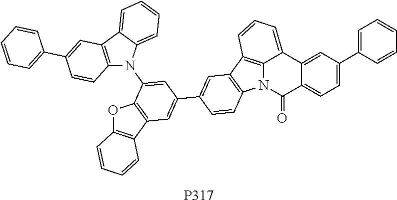 [SMILES: O=c1c2ccc(-c3ccccc3)cc2c2cccc3c4cc(-c5cc(-n6c7ccccc7c7cc(-c8ccccc8)ccc76)c6oc7ccccc7c6c5)ccc4n1c23 ; "P317"] |
| S139 | 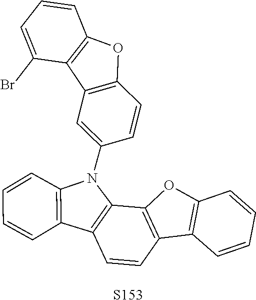 [SMILES: Brc1cccc2oc3ccc(-n4c5ccccc5c5ccc6c7ccccc7oc6c54)cc3c12 ; "S153"] | 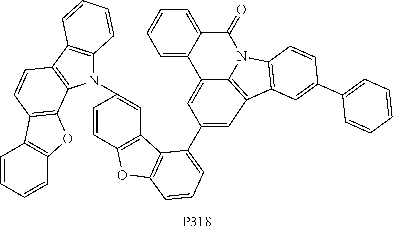 |
| S139 | 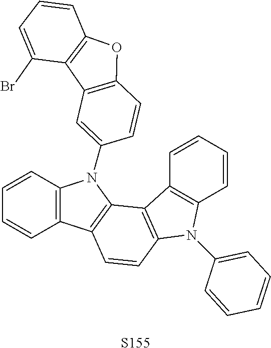 [SMILES: Brc1cccc2oc3ccc(-n4c5ccccc5c5ccc6c(c7ccccc7n6-c6ccccc6)c54)cc3c12 ; "S155"] | 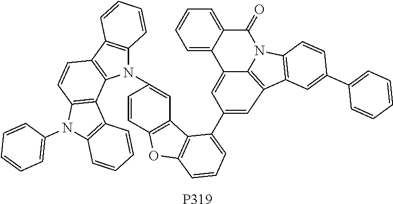 |
| S139 | 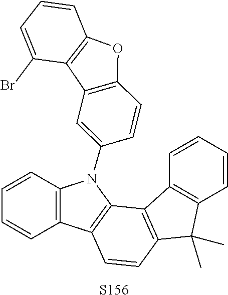 [SMILES: CC1(C)c2ccccc2-c2c1ccc1c3ccccc3n(-c3ccc4oc5cccc(Br)c5c4c3)c21 ; "S156"] | 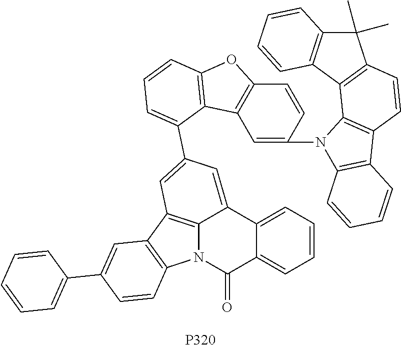 [SMILES: CC1(C)c2ccccc2-c2c1ccc1c3ccccc3n(-c3ccc4oc5cccc(-c6cc7c8ccccc8c(=O)n8c9ccc(-c%10ccccc%10)cc9c(c6)c78)c5c4c3)c21 ; "P320"] |
| S139 | 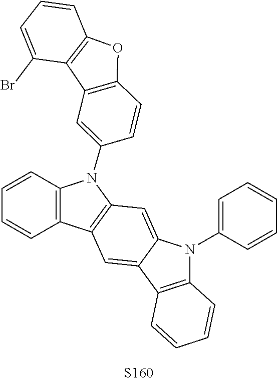 [SMILES: Brc1cccc2oc3ccc(-n4c5ccccc5c5cc6c7ccccc7n(-c7ccccc7)c6cc54)cc3c12 ; "S160"] | 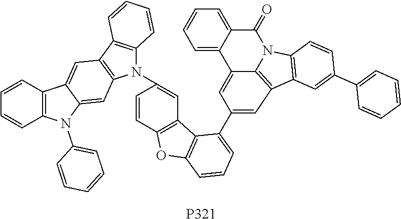 [SMILES: O=c1c2ccccc2c2cc(-c3cccc4oc5ccc(-n6c7ccccc7c7cc8c9ccccc9n(-c9ccccc9)c8cc76)cc5c34)cc3c4cc(-c5ccccc5)ccc4n1c23 ; "P321"] |
| S139 | 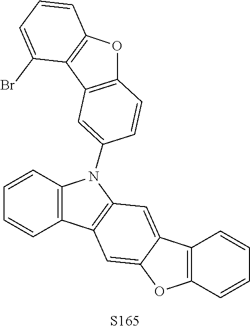 [SMILES: Brc1cccc2oc3ccc(-n4c5ccccc5c5cc6oc7ccccc7c6cc54)cc3c12 ; "S165"] | 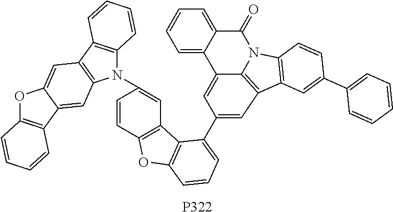 |
| S139 | 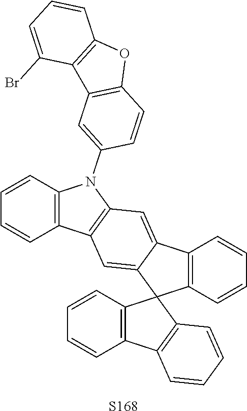 [SMILES: Brc1cccc2oc3ccc(-n4c5ccccc5c5cc6c(cc54)-c4ccccc4C64c5ccccc5-c5ccccc54)cc3c12 ; "S168"] | 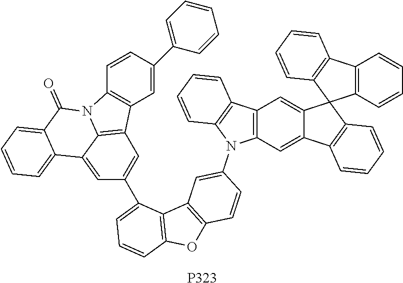 |
| S139 | 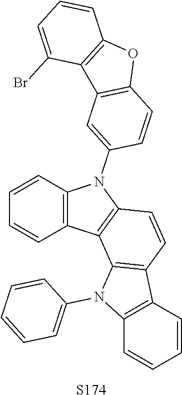 [SMILES: Brc1cccc2oc3ccc(-n4c5ccccc5c5c4ccc4c6ccccc6n(-c6ccccc6)c45)cc3c12 ; "S174"] | 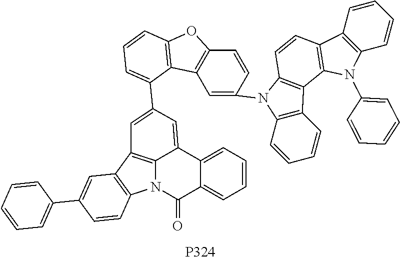 |
| S139 | 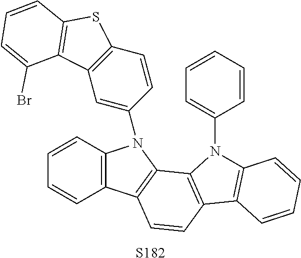 [SMILES: Brc1cccc2sc3ccc(-n4c5ccccc5c5ccc6c7ccccc7n(-c7ccccc7)c6c54)cc3c12 ; "S182"] | 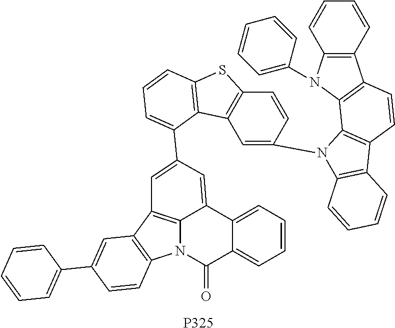 |
| S139 | 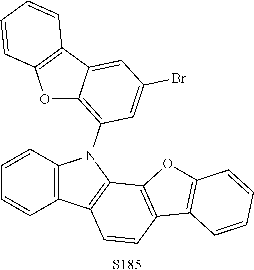 [SMILES: Brc1cc(-n2c3ccccc3c3ccc4c5ccccc5oc4c32)c2oc3ccccc3c2c1 ; "S185"] | 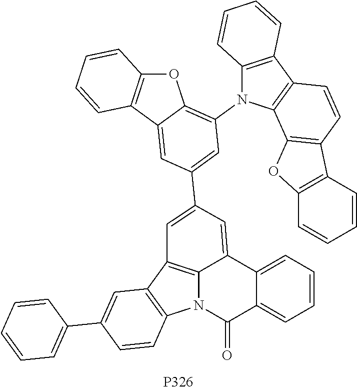 |
| S139 | 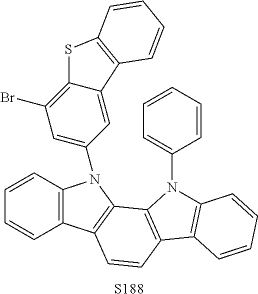 [SMILES: Brc1cc(-n2c3ccccc3c3ccc4c5ccccc5n(-c5ccccc5)c4c32)cc2c1sc1ccccc12 ; "S188"] | 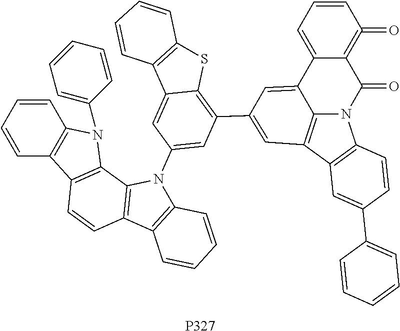 |
| S139 | 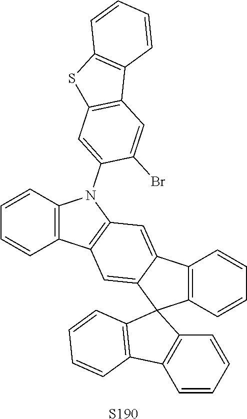 [SMILES: Brc1cc2c(cc1-n1c3ccccc3c3cc4c(cc31)-c1ccccc1C41c3ccccc3-c3ccccc31)sc1ccccc12 ; "S190"] | 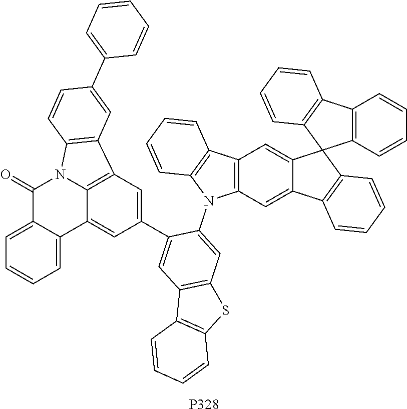 |
| S139 | 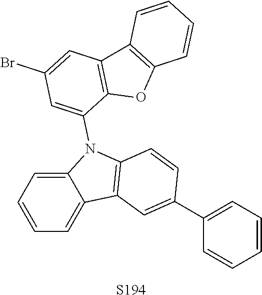 [SMILES: Brc1cc(-n2c3ccccc3c3cc(-c4ccccc4)ccc32)c2oc3ccccc3c2c1 ; "S194"] | 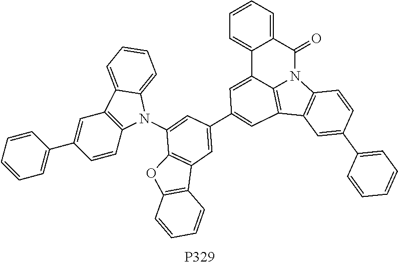 |
| S140 | 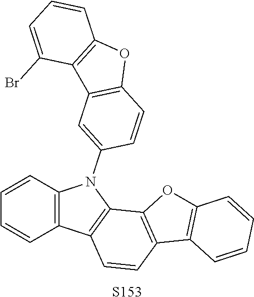 [SMILES: Brc1cccc2oc3ccc(-n4c5ccccc5c5ccc6c7ccccc7oc6c54)cc3c12 ; "S153"] | 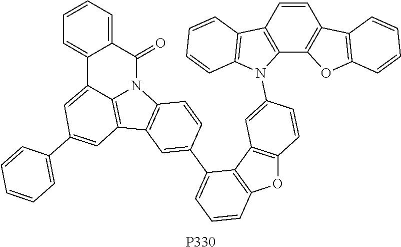 |
| S140 | 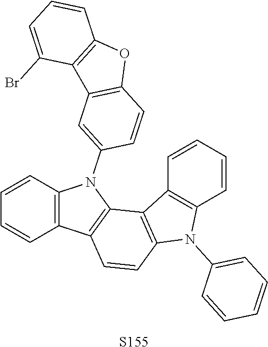 [SMILES: Brc1cccc2oc3ccc(-n4c5ccccc5c5ccc6c(c7ccccc7n6-c6ccccc6)c54)cc3c12 ; "S155"] | 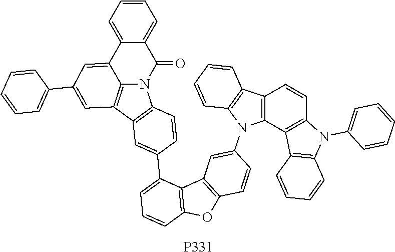 |
| S140 | 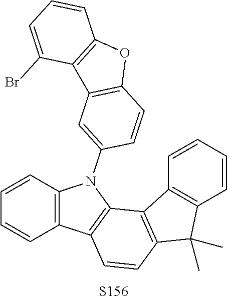 [SMILES: CC1(C)c2ccccc2-c2c1ccc1c3ccccc3n(-c3ccc4oc5cccc(Br)c5c4c3)c21 ; "S156"] | 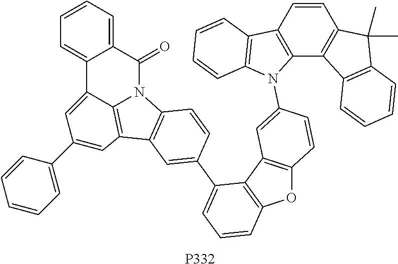 |
| S140 | 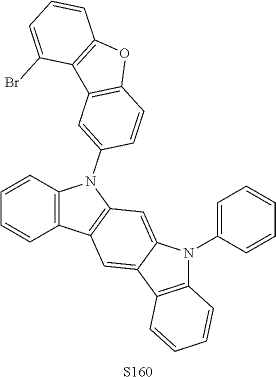 [SMILES: Brc1cccc2oc3ccc(-n4c5ccccc5c5cc6c7ccccc7n(-c7ccccc7)c6cc54)cc3c12 ; "S160"] | 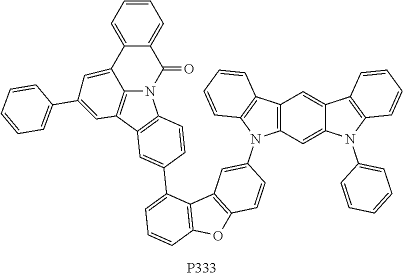 |
| S140 | 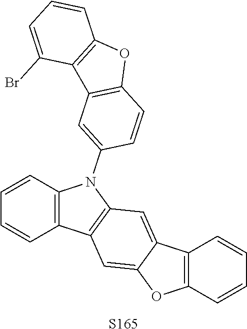 [SMILES: Brc1cccc2oc3ccc(-n4c5ccccc5c5cc6oc7ccccc7c6cc54)cc3c12 ; "S165"] | 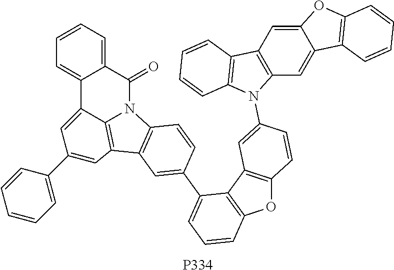 |
| S140 | 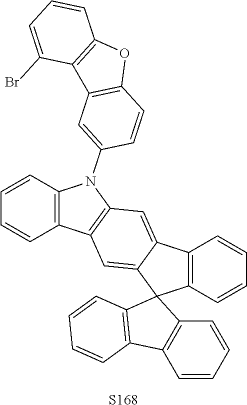 [SMILES: Brc1cccc2oc3ccc(-n4c5ccccc5c5cc6c(cc54)-c4ccccc4C64c5ccccc5-c5ccccc54)cc3c12 ; "S168"] | 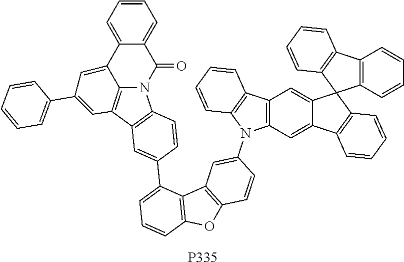 |
| S140 | 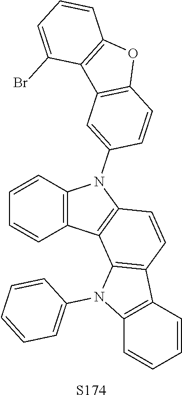 [SMILES: Brc1cccc2oc3ccc(-n4c5ccccc5c5c4ccc4c6ccccc6n(-c6ccccc6)c45)cc3c12 ; "S174"] | 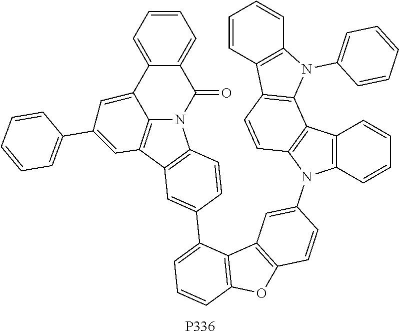 |
| S140 | 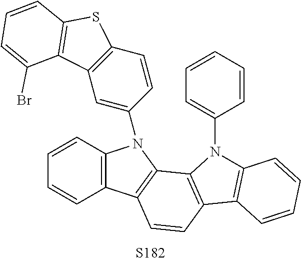 [SMILES: Brc1cccc2sc3ccc(-n4c5ccccc5c5ccc6c7ccccc7n(-c7ccccc7)c6c54)cc3c12 ; "S182"] | 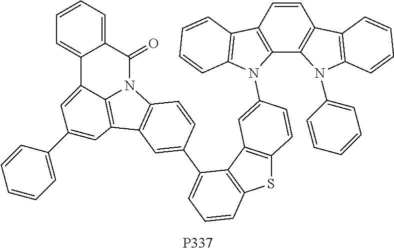 |
| S140 | 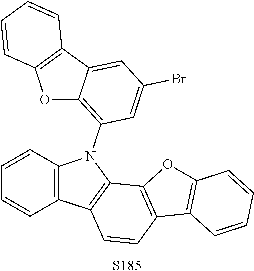 [SMILES: Brc1cc(-n2c3ccccc3c3ccc4c5ccccc5oc4c32)c2oc3ccccc3c2c1 ; "S185"] | 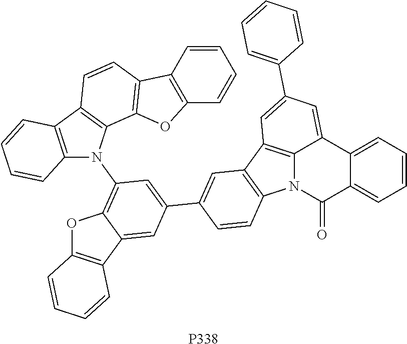 [SMILES: O=c1c2ccccc2c2cc(-c3ccccc3)cc3c4cc(-c5cc(-n6c7ccccc7c7ccc8c9ccccc9oc8c76)c6oc7ccccc7c6c5)ccc4n1c23 ; "P338"] |
| S140 | 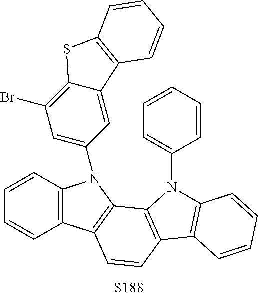 [SMILES: Brc1cc(-n2c3ccccc3c3ccc4c5ccccc5n(-c5ccccc5)c4c32)cc2c1sc1ccccc12 ; "S188"] | 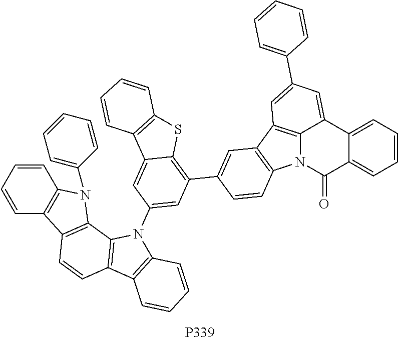 |
| S140 | 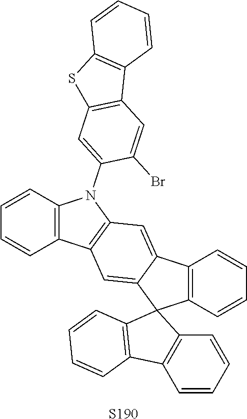 [SMILES: Brc1cc2c(cc1-n1c3ccccc3c3cc4c(cc31)-c1ccccc1C41c3ccccc3-c3ccccc31)sc1ccccc12 ; "S190"] | 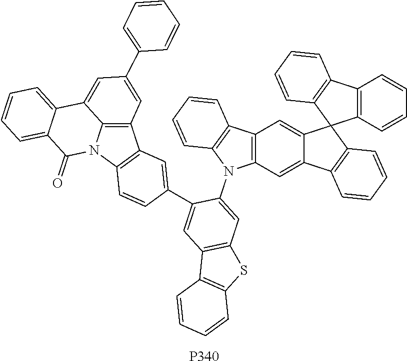 |
| S140 | 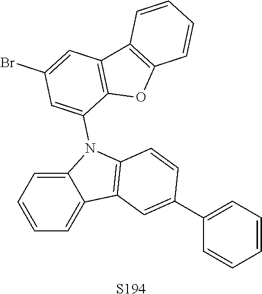 [SMILES: Brc1cc(-n2c3ccccc3c3cc(-c4ccccc4)ccc32)c2oc3ccccc3c2c1 ; "S194"] | 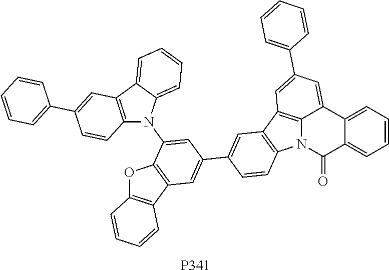 [SMILES: O=c1c2ccccc2c2cc(-c3ccccc3)cc3c4cc(-c5cc(-n6c7ccccc7c7cc(-c8ccccc8)ccc76)c6oc7ccccc7c6c5)ccc4n1c23 ; "P341"] |
| S128 | 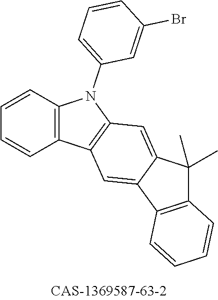 [SMILES: CC1(C)c2ccccc2-c2cc3c4ccccc4n(-c4cccc(Br)c4)c3cc21] | 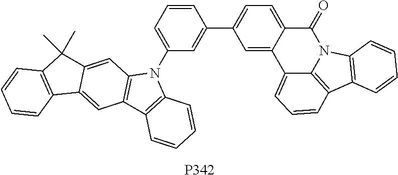 |
| S128 | 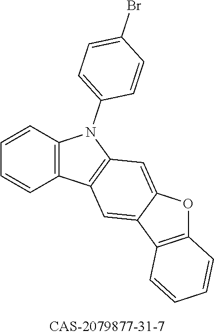 [SMILES: Brc1ccc(-n2c3ccccc3c3cc4c(cc32)oc2ccccc24)cc1] | 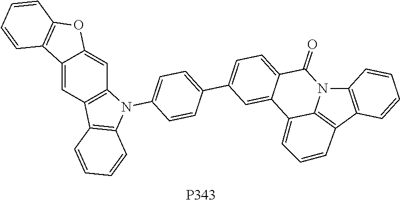 [SMILES: O=c1c2ccc(-c3ccc(-n4c5ccccc5c5cc6c(cc54)oc4ccccc46)cc3)cc2c2cccc3c4ccccc4n1c23 ; "P343"] |
| S128 | 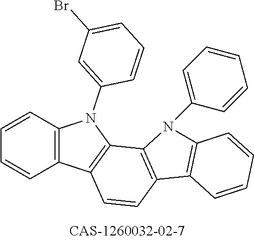 [SMILES: Brc1cccc(-n2c3ccccc3c3ccc4c5ccccc5n(-c5ccccc5)c4c32)c1] | 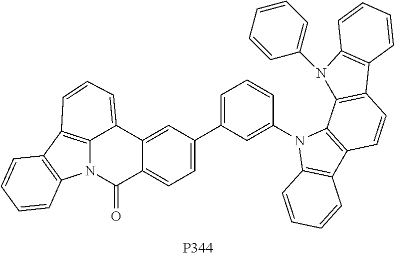 [SMILES: O=c1c2ccc(-c3cccc(-n4c5ccccc5c5ccc6c7ccccc7n(-c7ccccc7)c6c54)c3)cc2c2cccc3c4ccccc4n1c23 ; "P344"] |
| S128 | 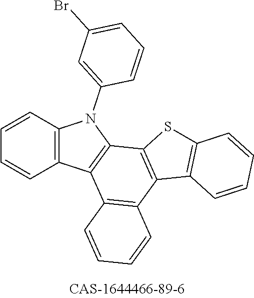 [SMILES: Brc1cccc(-n2c3ccccc3c3c4ccccc4c4c5ccccc5sc4c32)c1] | 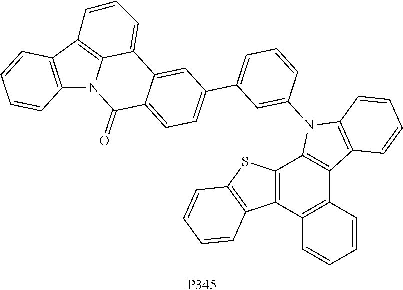 |
| S128 | 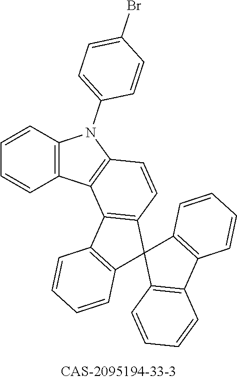 [SMILES: Brc1ccc(-n2c3ccccc3c3c4c(ccc32)C2(c3ccccc3-c3ccccc32)c2ccccc2-4)cc1] | 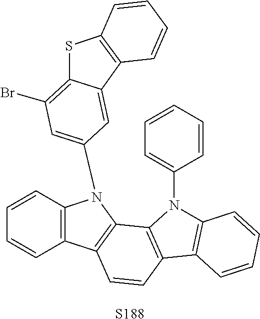 [SMILES: Brc1cc(-n2c3ccccc3c3ccc4c5ccccc5n(-c5ccccc5)c4c32)cc2c1sc1ccccc12 ; "S188"] |
| S128 | 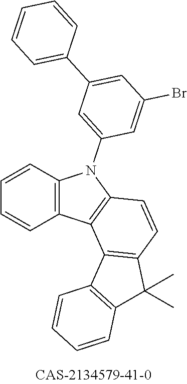 [SMILES: CC1(C)c2ccccc2-c2c1ccc1c2c2ccccc2n1-c1cc(Br)cc(-c2ccccc2)c1] | 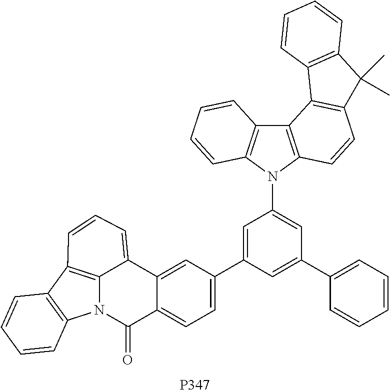 [SMILES: CC1(C)c2ccccc2-c2c1ccc1c2c2ccccc2n1-c1cc(-c2ccccc2)cc(-c2ccc3c(=O)n4c5ccccc5c5cccc(c3c2)c54)c1 ; "P347"] |
| S128 | 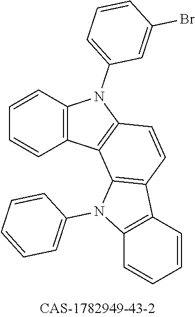 [SMILES: Brc1cccc(-n2c3ccccc3c3c2ccc2c4ccccc4n(-c4ccccc4)c23)c1] | 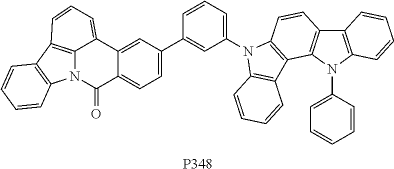 [SMILES: O=c1c2ccc(-c3cccc(-n4c5ccccc5c5c4ccc4c6ccccc6n(-c6ccccc6)c45)c3)cc2c2cccc3c4ccccc4n1c23 ; "P348"] |
| S128 | 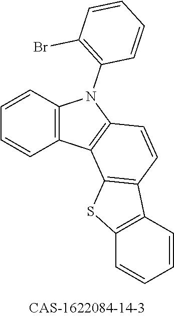 [SMILES: Brc1ccccc1-n1c2ccccc2c2c3sc4ccccc4c3ccc21] | 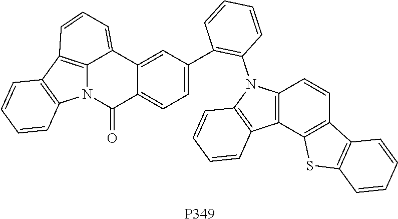 [SMILES: O=c1c2ccc(-c3ccccc3-n3c4ccccc4c4c5sc6ccccc6c5ccc43)cc2c2cccc3c4ccccc4n1c23 ; "P349"] |
| S128 | 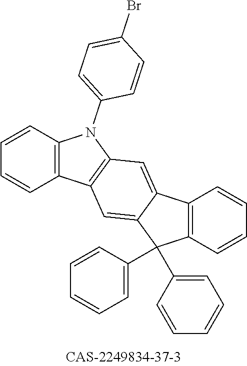 [SMILES: Brc1ccc(-n2c3ccccc3c3cc4c(cc32)-c2ccccc2C4(c2ccccc2)c2ccccc2)cc1] | 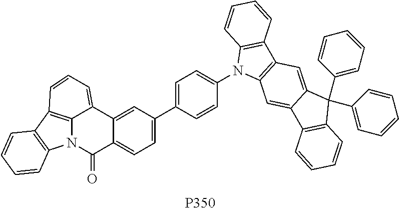 [SMILES: O=c1c2ccc(-c3ccc(-n4c5ccccc5c5cc6c(cc54)-c4ccccc4C6(c4ccccc4)c4ccccc4)cc3)cc2c2cccc3c4ccccc4n1c23 ; "P350"] |
| S128 | 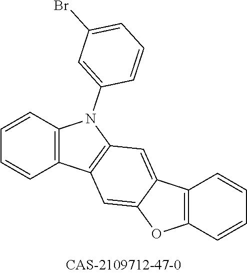 [SMILES: Brc1cccc(-n2c3ccccc3c3cc4oc5ccccc5c4cc32)c1] | 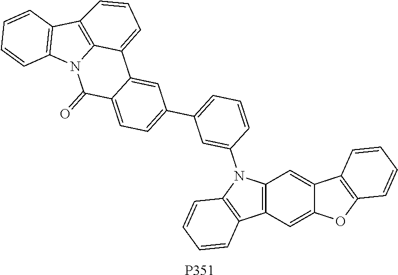 |
| S128 | 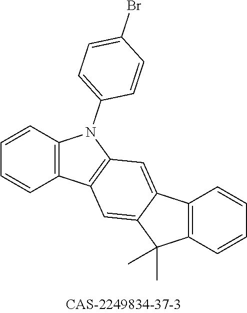 [SMILES: CC1(C)c2ccccc2-c2cc3c(cc21)c1ccccc1n3-c1ccc(Br)cc1] | 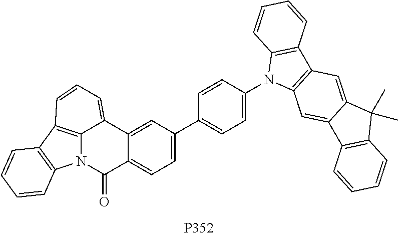 [SMILES: CC1(C)c2ccccc2-c2cc3c(cc21)c1ccccc1n3-c1ccc(-c2ccc3c(=O)n4c5ccccc5c5cccc(c3c2)c54)cc1 ; "P352"] |
| S128 | 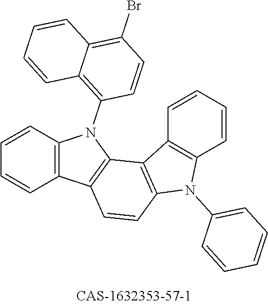 [SMILES: Brc1ccc(-n2c3ccccc3c3ccc4c(c5ccccc5n4-c4ccccc4)c32)c2ccccc12] | 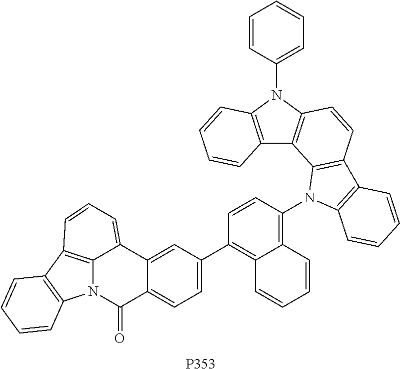 [SMILES: O=c1c2ccc(-c3ccc(-n4c5ccccc5c5ccc6c(c7ccccc7n6-c6ccccc6)c54)c4ccccc34)cc2c2cccc3c4ccccc4n1c23 ; "P353"] |
| S128 | 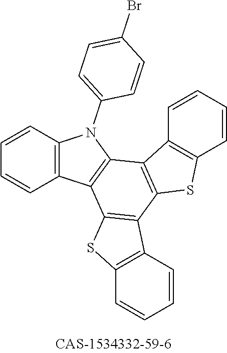 [SMILES: Brc1ccc(-n2c3ccccc3c3c4sc5ccccc5c4c4sc5ccccc5c4c32)cc1] | 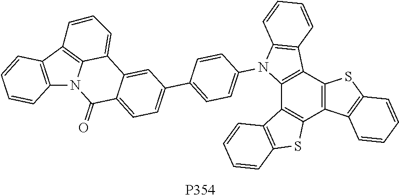 [SMILES: O=c1c2ccc(-c3ccc(-n4c5ccccc5c5c6sc7ccccc7c6c6sc7ccccc7c6c54)cc3)cc2c2cccc3c4ccccc4n1c23 ; "P354"] |
| S128 | 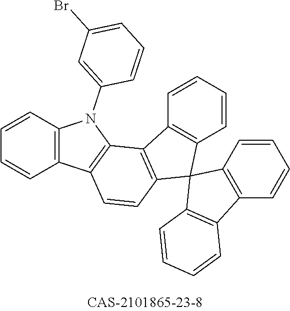 [SMILES: Brc1cccc(-n2c3ccccc3c3ccc4c(c32)-c2ccccc2C42c3ccccc3-c3ccccc32)c1] | 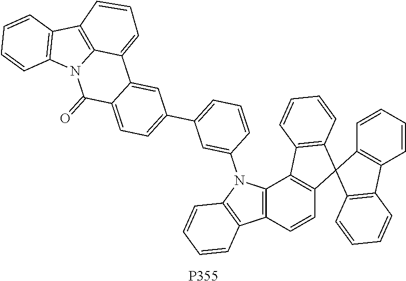 |
| S133 | 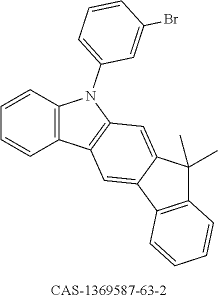 [SMILES: CC1(C)c2ccccc2-c2cc3c4ccccc4n(-c4cccc(Br)c4)c3cc21] | 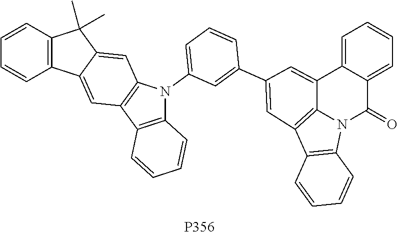 [SMILES: CC1(C)c2ccccc2-c2cc3c4ccccc4n(-c4cccc(-c5cc6c7ccccc7c(=O)n7c8ccccc8c(c5)c67)c4)c3cc21 ; "P356"] |
| S133 | 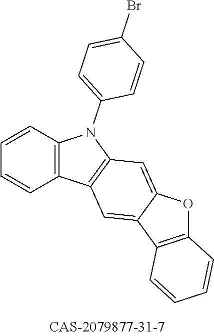 [SMILES: Brc1ccc(-n2c3ccccc3c3cc4c(cc32)oc2ccccc24)cc1] | 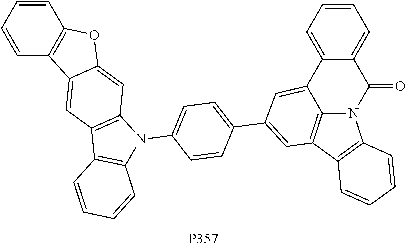 [SMILES: O=c1c2ccccc2c2cc(-c3ccc(-n4c5ccccc5c5cc6c(cc54)oc4ccccc46)cc3)cc3c4ccccc4n1c23 ; "P357"] |
| S133 | 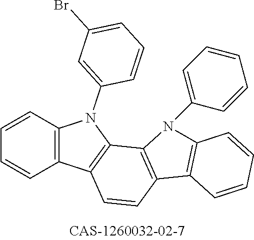 [SMILES: Brc1cccc(-n2c3ccccc3c3ccc4c5ccccc5n(-c5ccccc5)c4c32)c1] | 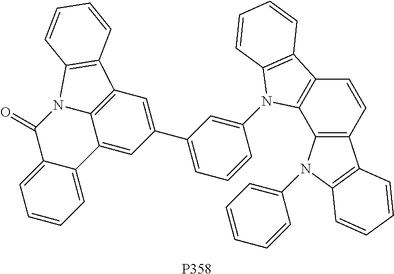 [SMILES: O=c1c2ccccc2c2cc(-c3cccc(-n4c5ccccc5c5ccc6c7ccccc7n(-c7ccccc7)c6c54)c3)cc3c4ccccc4n1c23 ; "P358"] |
| S133 | 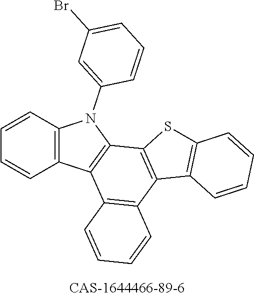 [SMILES: Brc1cccc(-n2c3ccccc3c3c4ccccc4c4c5ccccc5sc4c32)c1] | 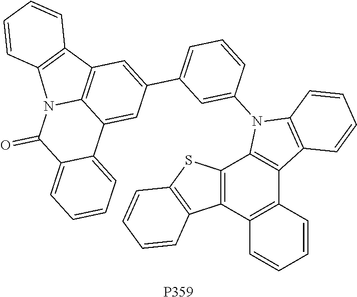 [SMILES: O=c1c2ccccc2c2cc(-c3cccc(-n4c5ccccc5c5c6ccccc6c6c7ccccc7sc6c54)c3)cc3c4ccccc4n1c23 ; "P359"] |
| S133 | 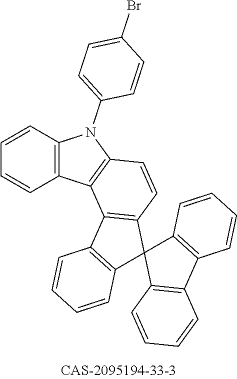 [SMILES: Brc1ccc(-n2c3ccccc3c3c4c(ccc32)C2(c3ccccc3-c3ccccc32)c2ccccc2-4)cc1] | 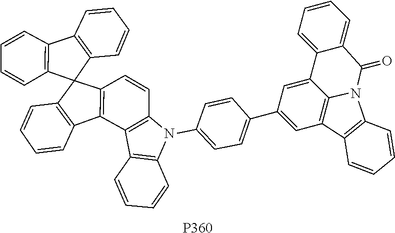 [SMILES: O=c1c2ccccc2c2cc(-c3ccc(-n4c5ccccc5c5c6c(ccc54)C4(c5ccccc5-c5ccccc54)c4ccccc4-6)cc3)cc3c4ccccc4n1c23 ; "P360"] |
| S133 | 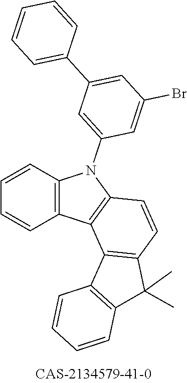 [SMILES: CC1(C)c2ccccc2-c2c1ccc1c2c2ccccc2n1-c1cc(Br)cc(-c2ccccc2)c1] | 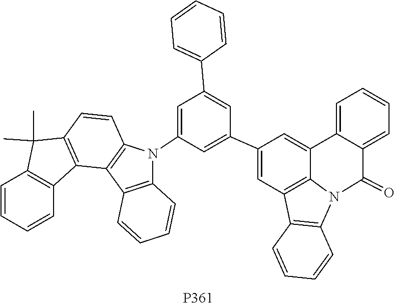 [SMILES: CC1(C)c2ccccc2-c2c1ccc1c2c2ccccc2n1-c1cc(-c2ccccc2)cc(-c2cc3c4ccccc4c(=O)n4c5ccccc5c(c2)c34)c1 ; "P361"] |
| S133 | 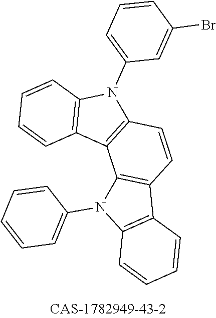 [SMILES: Brc1cccc(-n2c3ccccc3c3c2ccc2c4ccccc4n(-c4ccccc4)c23)c1] | 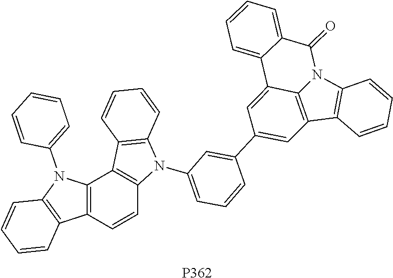 [SMILES: O=c1c2ccccc2c2cc(-c3cccc(-n4c5ccccc5c5c4ccc4c6ccccc6n(-c6ccccc6)c45)c3)cc3c4ccccc4n1c23 ; "P362"] |
| S133 | 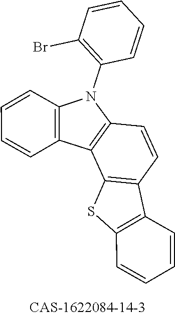 [SMILES: Brc1ccccc1-n1c2ccccc2c2c3sc4ccccc4c3ccc21] | 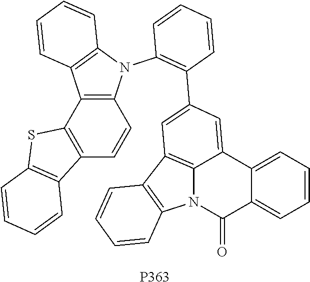 |
| S133 | 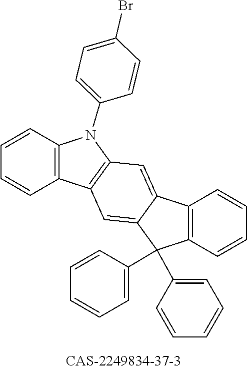 [SMILES: Brc1ccc(-n2c3ccccc3c3cc4c(cc32)-c2ccccc2C4(c2ccccc2)c2ccccc2)cc1] | 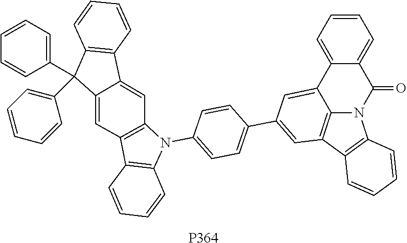 [SMILES: O=c1c2ccccc2c2cc(-c3ccc(-n4c5ccccc5c5cc6c(cc54)-c4ccccc4C6(c4ccccc4)c4ccccc4)cc3)cc3c4ccccc4n1c23 ; "P364"] |
| S133 | 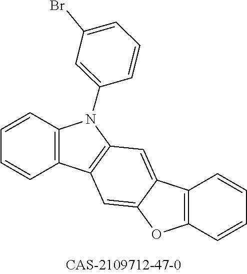 [SMILES: Brc1cccc(-n2c3ccccc3c3cc4oc5ccccc5c4cc32)c1] | 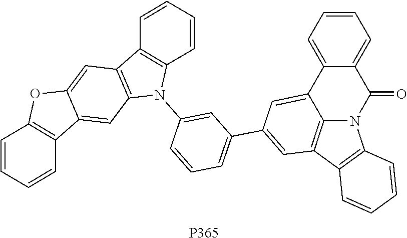 [SMILES: O=c1c2ccccc2c2cc(-c3cccc(-n4c5ccccc5c5cc6oc7ccccc7c6cc54)c3)cc3c4ccccc4n1c23 ; "P365"] |
| S133 | 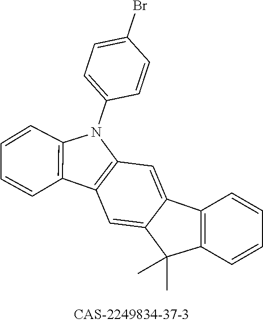 [SMILES: CC1(C)c2ccccc2-c2cc3c(cc21)c1ccccc1n3-c1ccc(Br)cc1] | 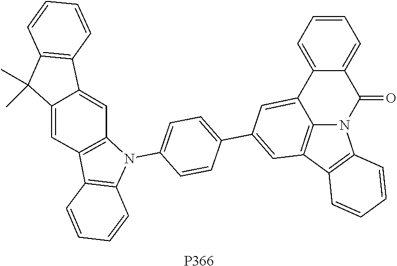 [SMILES: CC1(C)c2ccccc2-c2cc3c(cc21)c1ccccc1n3-c1ccc(-c2cc3c4ccccc4c(=O)n4c5ccccc5c(c2)c34)cc1 ; "P366"] |
| S133 | 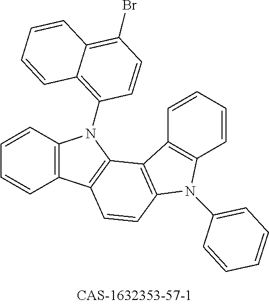 [SMILES: Brc1ccc(-n2c3ccccc3c3ccc4c(c5ccccc5n4-c4ccccc4)c32)c2ccccc12] | 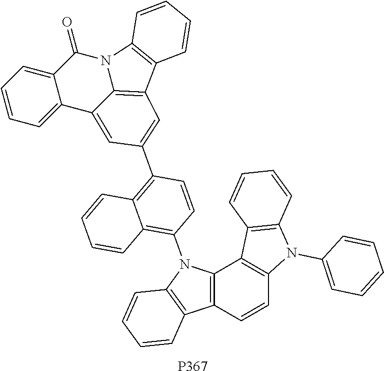 |
| S133 | 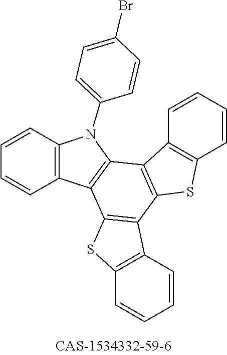 [SMILES: Brc1ccc(-n2c3ccccc3c3c4sc5ccccc5c4c4sc5ccccc5c4c32)cc1] | 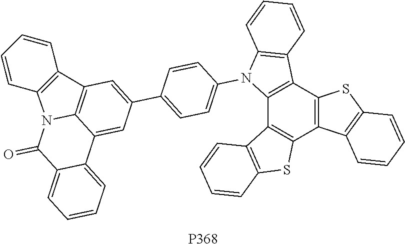 [SMILES: O=c1c2ccccc2c2cc(-c3ccc(-n4c5ccccc5c5c6sc7ccccc7c6c6sc7ccccc7c6c54)cc3)cc3c4ccccc4n1c23 ; "P368"] |
| S133 | 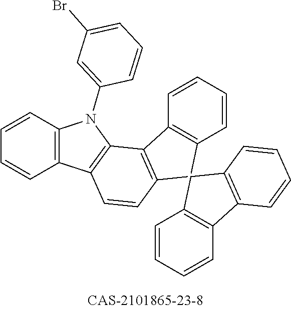 | 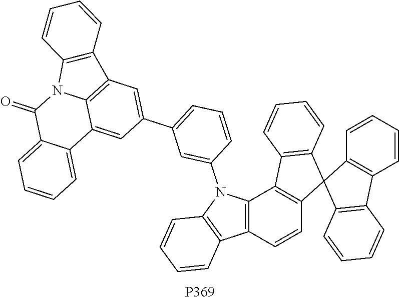 |
| S135 | 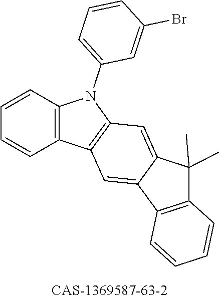 [SMILES: CC1(C)c2ccccc2-c2cc3c4ccccc4n(-c4cccc(Br)c4)c3cc21] | 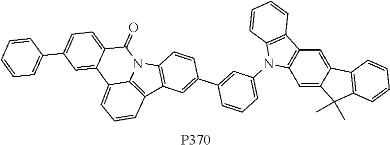 [SMILES: CC1(C)c2ccccc2-c2cc3c4ccccc4n(-c4cccc(-c5ccc6c(c5)c5cccc7c8cc(-c9ccccc9)ccc8c(=O)n6c75)c4)c3cc21 ; "P370"] |
| S135 | 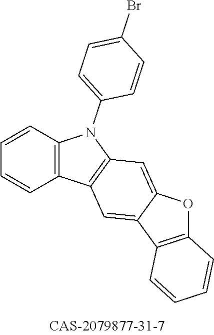 [SMILES: Brc1ccc(-n2c3ccccc3c3cc4c(cc32)oc2ccccc24)cc1] | 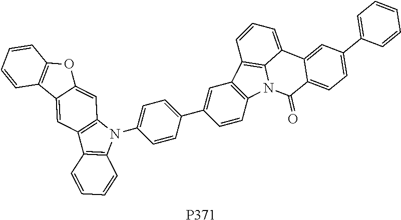 [SMILES: O=c1c2ccc(-c3ccccc3)cc2c2cccc3c4cc(-c5ccc(-n6c7ccccc7c7cc8c(cc76)oc6ccccc68)cc5)ccc4n1c23 ; "P371"] |
| S135 | 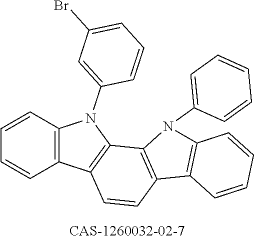 [SMILES: Brc1cccc(-n2c3ccccc3c3ccc4c5ccccc5n(-c5ccccc5)c4c32)c1] | 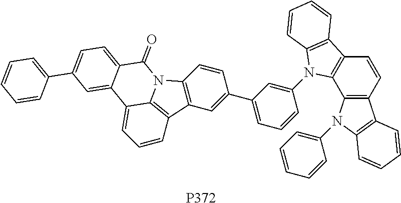 [SMILES: O=c1c2ccc(-c3ccccc3)cc2c2cccc3c4cc(-c5cccc(-n6c7ccccc7c7ccc8c9ccccc9n(-c9ccccc9)c8c76)c5)ccc4n1c23 ; "P372"] |
| S135 | 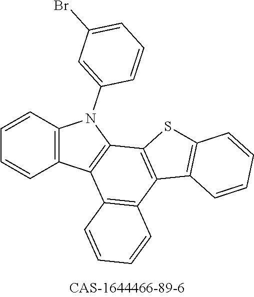 [SMILES: Brc1cccc(-n2c3ccccc3c3c4ccccc4c4c5ccccc5sc4c32)c1] | 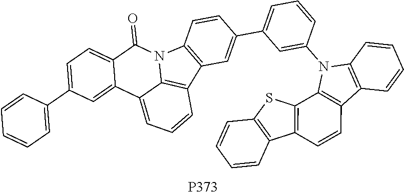 [SMILES: O=c1c2ccc(-c3ccccc3)cc2c2cccc3c4cc(-c5cccc(-n6c7ccccc7c7ccc8c9ccccc9sc8c76)c5)ccc4n1c23 ; "P373"] |
| S135 | 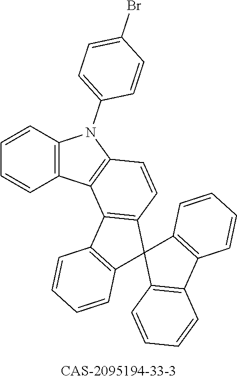 [SMILES: Brc1ccc(-n2c3ccccc3c3c4c(ccc32)C2(c3ccccc3-c3ccccc32)c2ccccc2-4)cc1] | 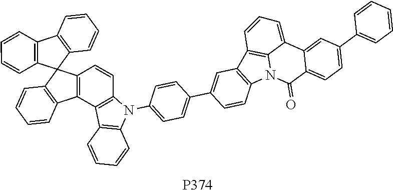 [SMILES: O=c1c2ccc(-c3ccccc3)cc2c2cccc3c4cc(-c5ccc(-n6c7ccccc7c7c8c(ccc76)C6(c7ccccc7-c7ccccc76)c6ccccc6-8)cc5)ccc4n1c23 ; "P374"] |
| S135 | 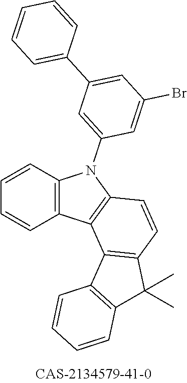 [SMILES: CC1(C)c2ccccc2-c2c1ccc1c2c2ccccc2n1-c1cc(Br)cc(-c2ccccc2)c1] | 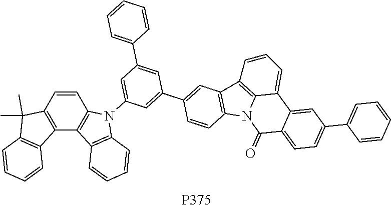 [SMILES: CC1(C)c2ccccc2-c2c1ccc1c2c2ccccc2n1-c1cc(-c2ccccc2)cc(-c2ccc3c(c2)c2cccc4c5cc(-c6ccccc6)ccc5c(=O)n3c42)c1 ; "P375"] |
| S135 | 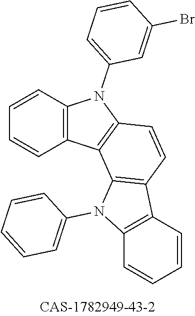 [SMILES: Brc1cccc(-n2c3ccccc3c3c2ccc2c4ccccc4n(-c4ccccc4)c23)c1] | 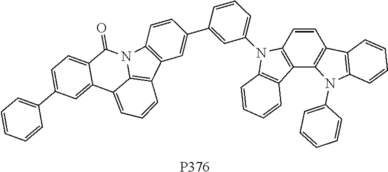 [SMILES: O=c1c2ccc(-c3ccccc3)cc2c2cccc3c4cc(-c5cccc(-n6c7ccccc7c7c6ccc6c8ccccc8n(-c8ccccc8)c67)c5)ccc4n1c23 ; "P376"] |
| S135 | 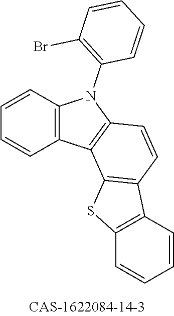 [SMILES: Brc1ccccc1-n1c2ccccc2c2c3sc4ccccc4c3ccc21] | 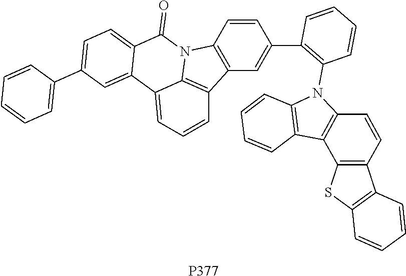 [SMILES: O=c1c2ccc(-c3ccccc3)cc2c2cccc3c4cc(-c5ccccc5-n5c6ccccc6c6c7sc8ccccc8c7ccc65)ccc4n1c23 ; "P377"] |
| S135 | 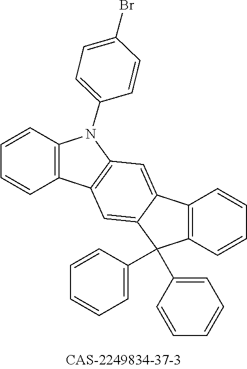 [SMILES: Brc1ccc(-n2c3ccccc3c3cc4c(cc32)-c2ccccc2C4(c2ccccc2)c2ccccc2)cc1] | 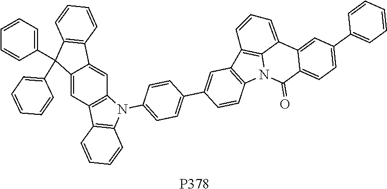 [SMILES: O=c1c2ccc(-c3ccccc3)cc2c2cccc3c4cc(-c5ccc(-n6c7ccccc7c7cc8c(cc76)-c6ccccc6C8(c6ccccc6)c6ccccc6)cc5)ccc4n1c23 ; "P378"] |
| S135 | 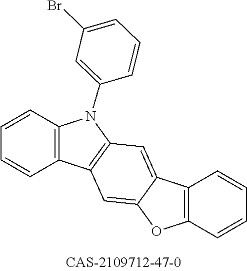 [SMILES: Brc1cccc(-n2c3ccccc3c3cc4oc5ccccc5c4cc32)c1] | 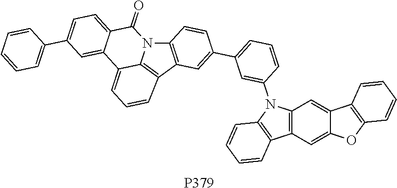 [SMILES: O=c1c2ccc(-c3ccccc3)cc2c2cccc3c4cc(-c5cccc(-n6c7ccccc7c7cc8oc9ccccc9c8cc76)c5)ccc4n1c23 ; "P379"] |
| S135 | 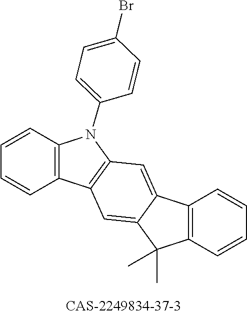 [SMILES: CC1(C)c2ccccc2-c2cc3c(cc21)c1ccccc1n3-c1ccc(Br)cc1] | 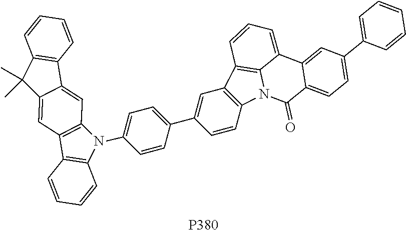 [SMILES: CC1(C)c2ccccc2-c2cc3c(cc21)c1ccccc1n3-c1ccc(-c2ccc3c(c2)c2cccc4c5cc(-c6ccccc6)ccc5c(=O)n3c42)cc1 ; "P380"] |
| S135 | 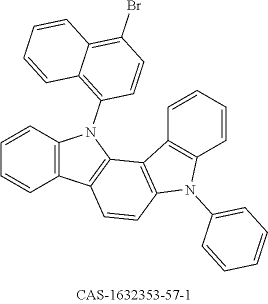 [SMILES: Brc1ccc(-n2c3ccccc3c3ccc4c(c5ccccc5n4-c4ccccc4)c32)c2ccccc12] | 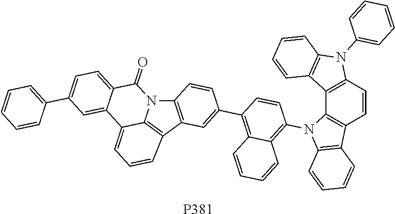 [SMILES: O=c1c2ccc(-c3ccccc3)cc2c2cccc3c4cc(-c5ccc(-n6c7ccccc7c7ccc8c(c9ccccc9n8-c8ccccc8)c76)c6ccccc56)ccc4n1c23 ; "P381"] |
| S135 | 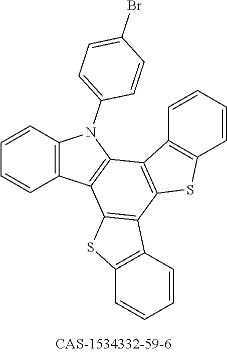 [SMILES: Brc1ccc(-n2c3ccccc3c3c4sc5ccccc5c4c4sc5ccccc5c4c32)cc1] | 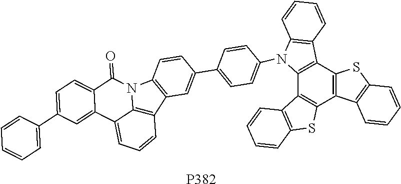 [SMILES: O=c1c2ccc(-c3ccccc3)cc2c2cccc3c4cc(-c5ccc(-n6c7ccccc7c7c8sc9ccccc9c8c8sc9ccccc9c8c76)cc5)ccc4n1c23 ; "P382"] |
| S135 | 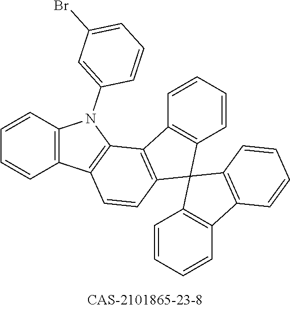 [SMILES: Brc1cccc(-n2c3ccccc3c3ccc4c(c32)-c2ccccc2C42c3ccccc3-c3ccccc32)c1] | 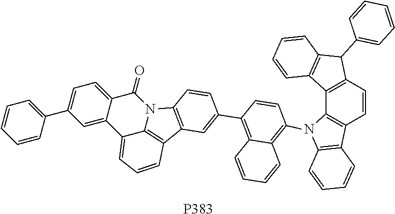 [SMILES: O=c1c2ccc(-c3ccccc3)cc2c2cccc3c4cc(-c5ccc(-n6c7ccccc7c7ccc8c(c76)-c6ccccc6C8c6ccccc6)c6ccccc56)ccc4n1c23 ; "P383"] |
| S125 | 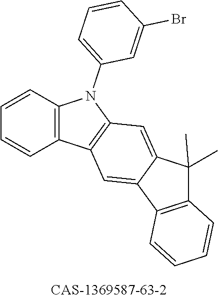 [SMILES: CC1(C)c2ccccc2-c2cc3c4ccccc4n(-c4cccc(Br)c4)c3cc21] | 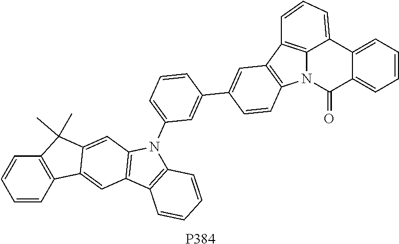 |
| S125 | 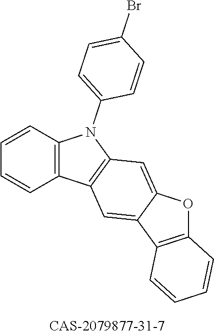 [SMILES: Brc1ccc(-n2c3ccccc3c3cc4c(cc32)oc2ccccc24)cc1] | 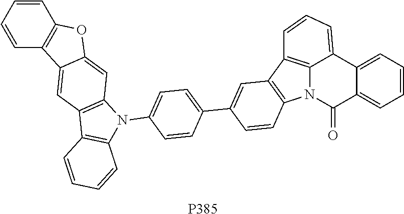 [SMILES: O=c1c2ccccc2c2cccc3c4cc(-c5ccc(-n6c7ccccc7c7cc8c(cc76)oc6ccccc68)cc5)ccc4n1c23 ; "P385"] |
| S125 | 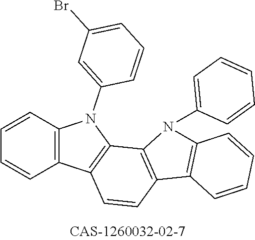 [SMILES: Brc1cccc(-n2c3ccccc3c3ccc4c5ccccc5n(-c5ccccc5)c4c32)c1] | 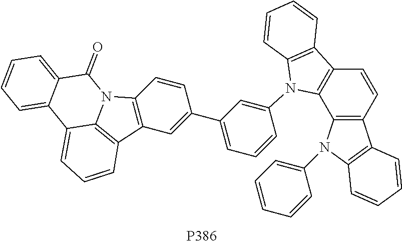 [SMILES: O=c1c2ccccc2c2cccc3c4cc(-c5cccc(-n6c7ccccc7c7ccc8c9ccccc9n(-c9ccccc9)c8c76)c5)ccc4n1c23 ; "P386"] |
| S125 | 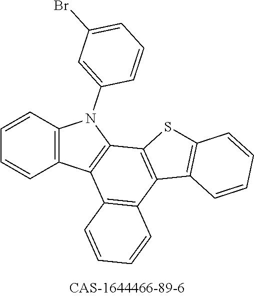 [SMILES: Brc1cccc(-n2c3ccccc3c3c4ccccc4c4c5ccccc5sc4c32)c1] | 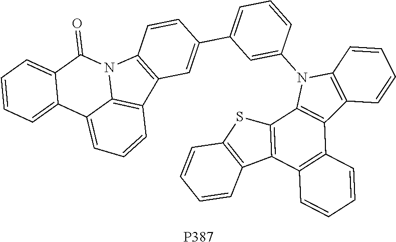 |
| S125 | 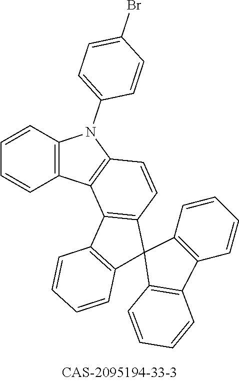 [SMILES: Brc1ccc(-n2c3ccccc3c3c4c(ccc32)C2(c3ccccc3-c3ccccc32)c2ccccc2-4)cc1] | 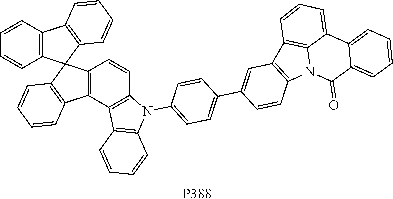 [SMILES: O=c1c2ccccc2c2cccc3c4cc(-c5ccc(-n6c7ccccc7c7c8c(ccc76)C6(c7ccccc7-c7ccccc76)c6ccccc6-8)cc5)ccc4n1c23 ; "P388"] |
| S125 | 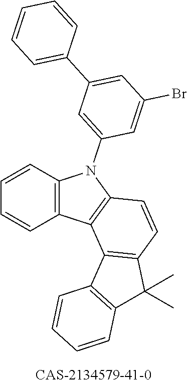 [SMILES: CC1(C)c2ccccc2-c2c1ccc1c2c2ccccc2n1-c1cc(Br)cc(-c2ccccc2)c1] | 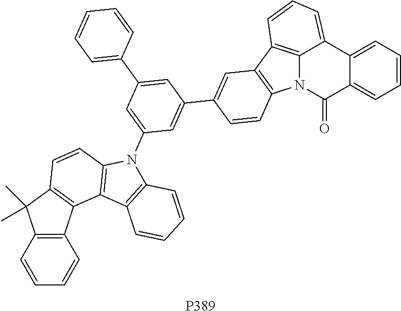 [SMILES: CC1(C)c2ccccc2-c2c1ccc1c2c2ccccc2n1-c1cc(-c2ccccc2)cc(-c2ccc3c(c2)c2cccc4c5ccccc5c(=O)n3c42)c1 ; "P389"] |
| S125 | 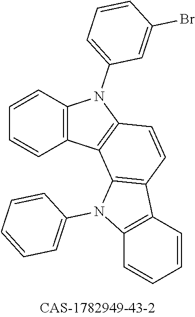 [SMILES: Brc1cccc(-n2c3ccccc3c3c2ccc2c4ccccc4n(-c4ccccc4)c23)c1] | 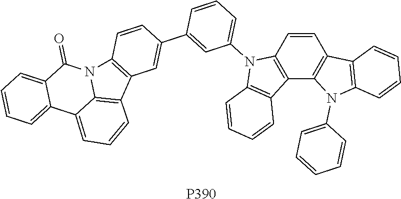 [SMILES: O=c1c2ccccc2c2cccc3c4cc(-c5cccc(-n6c7ccccc7c7c6ccc6c8ccccc8n(-c8ccccc8)c67)c5)ccc4n1c23 ; "P390"] |
| S125 | 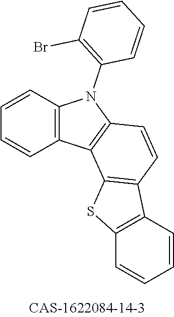 [SMILES: Brc1ccccc1-n1c2ccccc2c2c3sc4ccccc4c3ccc21] | 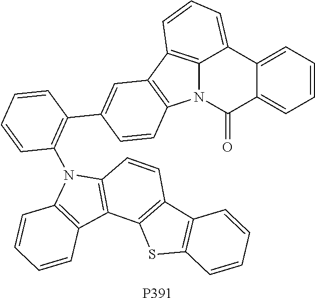 |
| S125 | 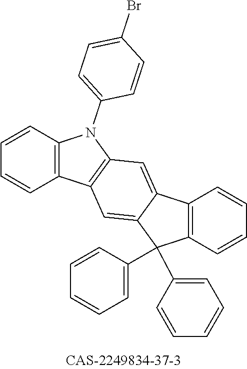 [SMILES: Brc1ccc(-n2c3ccccc3c3cc4c(cc32)-c2ccccc2C4(c2ccccc2)c2ccccc2)cc1] | 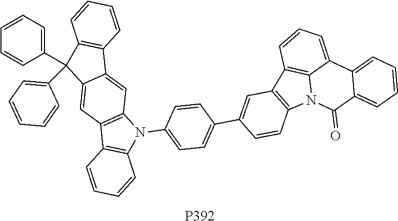 [SMILES: O=c1c2ccccc2c2cccc3c4cc(-c5ccc(-n6c7ccccc7c7cc8c(cc76)-c6ccccc6C8(c6ccccc6)c6ccccc6)cc5)ccc4n1c23 ; "P392"] |
| S125 | 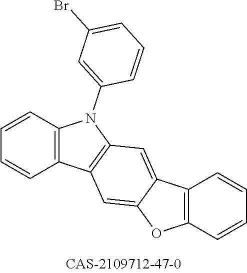 [SMILES: Brc1cccc(-n2c3ccccc3c3cc4oc5ccccc5c4cc32)c1] | 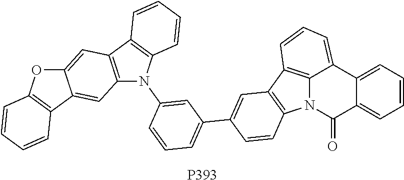 |
| S125 | 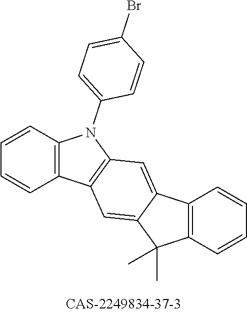 [SMILES: CC1(C)c2ccccc2-c2cc3c(cc21)c1ccccc1n3-c1ccc(Br)cc1] | 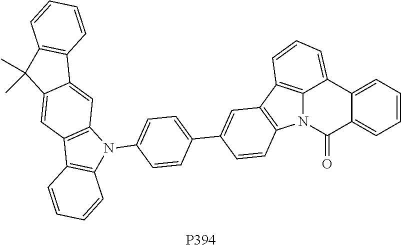 [SMILES: CC1(C)c2ccccc2-c2cc3c(cc21)c1ccccc1n3-c1ccc(-c2ccc3c(c2)c2cccc4c5ccccc5c(=O)n3c42)cc1 ; "P394"] |
| S125 | 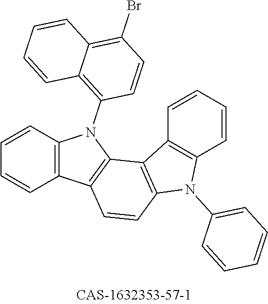 [SMILES: Brc1ccc(-n2c3ccccc3c3ccc4c(c5ccccc5n4-c4ccccc4)c32)c2ccccc12] | 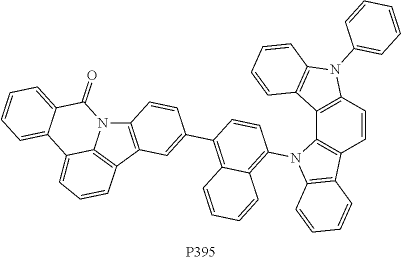 [SMILES: O=c1c2ccccc2c2cccc3c4cc(-c5ccc(-n6c7ccccc7c7ccc8c(c9ccccc9n8-c8ccccc8)c76)c6ccccc56)ccc4n1c23 ; "P395"] |
| S125 | 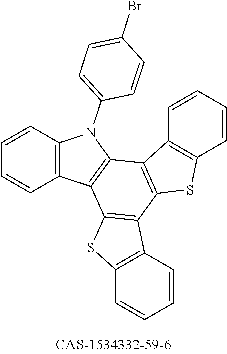 [SMILES: Brc1ccc(-n2c3ccccc3c3c4sc5ccccc5c4c4sc5ccccc5c4c32)cc1] | 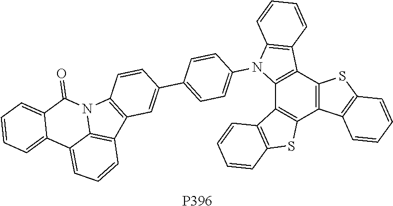 [SMILES: O=c1c2ccccc2c2cccc3c4cc(-c5ccc(-n6c7ccccc7c7c8sc9ccccc9c8c8sc9ccccc9c8c76)cc5)ccc4n1c23 ; "P396"] |
| S125 | 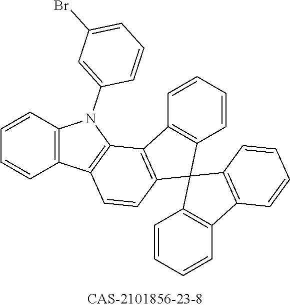 [SMILES: Brc1cccc(-n2c3ccccc3c3ccc4c(c32)-c2ccccc2C42c3ccccc3-c3ccccc32)c1] | 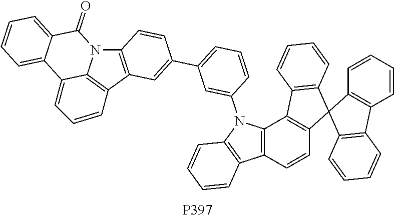 |

### Production of the OLEDs

Examples C1 to 118 which follow (see Table 1) present the use of the materials of the invention in OLEDs.

Pretreatment for Examples C1-118: Glass plaques coated with structured ITO (indium tin oxide) of thickness 50 nm are treated prior to coating, first with an oxygen plasma, followed by an argon plasma. These plasma-treated glass plaques form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminium layer of thickness 100 nm. The exact structure of the OLEDs can be found in table 1. The materials required for production of the OLEDs are shown in Table 3. The device data of the OLEDs are listed in Table 2. Examples C1 to C4 are comparative examples according to the prior art, examples 11 to 118 show data of OLEDs of the invention.

All materials are applied by thermal vapour deposition in a vacuum chamber. In this case, the emission layer always consists of at least one matrix material (host material) and an emitting dopant (emitter) which is added to the matrix material(s) in a particular proportion by volume by co-evaporation. Details given in such a form as SoA1:CoH1:TEG1 (45%:45%:10%) mean here that the material SoA1 is present in the layer in a proportion by volume of 45%, CoH1 in a proportion by volume of 45% and TEG1 in a proportion by volume of 10%. Analogously, the electron transport layer may also consist of a mixture of two materials.

The OLEDs are characterized in a standard manner. For this purpose, the electroluminescence spectra and the current efficiency (SE, measured in cd/A) as a function of luminance, calculated from current-voltage-luminance characteristics assuming Lambertian radiation characteristics, and the lifetime are measured. The electroluminescence spectra are determined at a current density of 10 mA/cm², and the CIE 1931 x and y colour coordinates are calculated therefrom. The lifetime LT is defined as the time after which the luminance drops from the starting luminance to a certain proportion L1 in the course of operation with constant current density j₀. A figure of L1=80% in Table 2 means that the lifetime reported in the LT column corresponds to the time after which the luminance falls to 80% of its starting value.

### Use of Materials of the Invention in OLEDs

The materials of the invention can be used in the emission layer in phosphorescent green OLEDs. Inventive compounds P1, P9, P13, P28, P35, P67, P88, P205, P213, P218, P229, P247, P251, P332, P393 with or without CoH1 or CoH2 are used in Examples 11 to 118 as matrix material in the emission layer. The examples are elucidated in detail hereinafter, in order to illustrate the advantages of the OLEDs of the invention.

### Use of Materials of the Invention in the Emission Layer of Phosphorescent OLEDs

The use of the inventive compounds P1 and P9 as matrix material in the emission layer (Examples 11 to 14) can achieve a distinct improvement in lifetime compared to the prior art compounds (Examples C1 to C4). By combination of P1, P9, P13, P28, P35, P67, P88, P205, P213, P218, P229, P247, P251, P332, P393 with CoH1 or with CoH2 and TEG1 or TEG2, it is additionally possible to further distinctly enhance the lifetime.

TABLE 1
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| C1 | HATCN | SpMA1 | SpMA2 | SoA1:TEG1 | ST2 | ST2:LiQ | LiQ 1 nm |
| | 5 nm | 230 nm | 20 nm | (88%:12%) | 10 nm | (50%:50%) | |
| | | | | 30 nm | | 30 nm | |
| C2 | HATCN | SpMA1 | SpMA2 | SoA2:TEG1 | ST2 | ST2:LiQ | LiQ 1 nm |
| | 5 nm | 230 nm | 20 nm | (88%:12%) | 10 nm | (50%:50%) | |
| | | | | 30 nm | | 30 nm | |
| I1 | HATCN | SpMA1 | SpMA2 | P1:TEG1 | ST2 | ST2:LiQ | LiQ 1 nm |
| | 5 nm | 230 nm | 20 nm | (88%:12%) | 10 nm | (50%:50%) | |
| | | | | 30 nm | | 30 nm | |
| I2 | HATCN | SpMA1 | SpMA2 | P9:TEG1 | ST2 | ST2:LiQ | LiQ 1 nm |
| | 5 nm | 230 nm | 20 nm | (88%:12%) | 10 nm | (50%:50%) | |
| | | | | 30 nm | | 30 nm | |
| C3 | HATCN | SpMA1 | SpMA2 | SoA1:CoH1:TEG1 | ST2 | ST2:LiQ | LiQ 1 nm |
| | 5 nm | 230 nm | 20 nm | (59%:29%:12%) | 10 nm | (50%:50%) | |
| | | | | 30 nm | | 30 nm | |
| C4 | HATCN | SpMA1 | SpMA2 | SoA1:CoH1:TEG1 | ST2 | ST2:LiQ | LiQ 1 nm |
| | 5 nm | 230 nm | 20 nm | (59%:29%:12%) | 10 nm | (50%:50%) | |
| | | | | 30 nm | | 30 nm | |
| I3 | HATCN | SpMA1 | SpMA2 | P1:CoH1:TEG1 | ST2 | ST2:LiQ | LiQ 1 nm |
| | 5 nm | 230 nm | 20 nm | (59%:29%:12%) | 10 nm | (50%:50%) | |
| | | | | 30 nm | | 30 nm | |
| I4 | HATCN | SpMA1 | SpMA2 | P9:CoH1:TEG1 | ST2 | ST2:LiQ | LiQ 1 nm |
| | 5 nm | 230 nm | 20 nm | (59%:29%:12%) | 10 nm | (50%:50%) | |
| | | | | 30 nm | | 30 nm | |
| I5 | HATCN | SpMA1 | SpMA2 | P1:CoH2:TEG2 | ST2 | ST2:LiQ | LiQ 1 nm |
| | 5 nm | 230 nm | 20 nm | (44%:44%:12%) | 10 nm | (50%:50%) | |
| | | | | 30 nm | | 30 nm | |
| I6 | HATCN | SpMA1 | SpMA2 | P13:CoH1:TEG1 | ST2 | ST2:LiQ | LiQ 1 nm |
| | 5 nm | 230 nm | 20 nm | (59%:29%:12%) | 10 nm | (50%:50%) | |
| | | | | 30 nm | | 30 nm | |
| I7 | HATCN | SpMA1 | SpMA2 | P28:CoH2:TEG2 | ST2 | ST2:LiQ | LiQ 1 nm |
| | 5 nm | 230 nm | 20 nm | (44%:44%:12%) | 10 nm | (50%:50%) | |
| | | | | 30 nm | | 30 nm | |
| I8 | HATCN | SpMA1 | SpMA2 | P35:CoH2:TEG2 | ST2 | ST2:LiQ | LiQ 1 nm |
| | 5 nm | 230 nm | 20 nm | (44%:44%:12%) | 10 nm | (50%:50%) | |
| | | | | 30 nm | | 30 nm | |
| I9 | HATCN | SpMA1 | SpMA2 | P67:CoH1:TEG1 | ST2 | ST2:LiQ | LiQ 1 nm |
| | 5 nm | 230 nm | 20 nm | (59%:29%:12%) | 10 nm | (50%:50%) | |
| | | | | 30 nm | | 30 nm | |
| I10 | HATCN | SpMA1 | SpMA2 | P88:CoH1:TEG1 | ST2 | ST2:LiQ | LiQ 1 nm |
| | 5 nm | 230 nm | 20 nm | (59%:29%:12%) | 10 nm | (50%:50%) | |
| | | | | 30 nm | | 30 nm | |
| I11 | HATCN | SpMA1 | SpMA2 | P205:CoH2:TEG2 | ST2 | ST2:LiQ | LiQ 1 nm |
| | 5 nm | 230 nm | 20 nm | (44%:44%:12%) | 10 nm | (50%:50%) | |
| | | | | 30 nm | | 30 nm | |
| I12 | HATCN | SpMA1 | SpMA2 | P213:CoH1:TEG1 | ST2 | ST2:LiQ | LiQ 1 nm |
| | 5 nm | 230 nm | 20 nm | (59%:29%:12%) | 10 nm | (50%:50%) | |
| | | | | 30 nm | | 30 nm | |
| I13 | HATCN | SpMA1 | SpMA2 | P218:CoH2:TEG2 | ST2 | ST2:LiQ | LiQ 1 nm |
| | 5 nm | 230 nm | 20 nm | (44%:44%:12%) | 10 nm | (50%:50%) | |
| | | | | 30 nm | | 30 nm | |
| I14 | HATCN | SpMA1 | SpMA2 | P229:CoH1:TEG1 | ST2 | ST2:LiQ | LiQ 1 nm |
| | 5 nm | 230 nm | 20 nm | (59%:29%:12%) | 10 nm | (50%:50%) | |
| | | | | 30 nm | | 30 nm | |
| I15 | HATCN | SpMA1 | SpMA2 | P247:CoH2:TEG2 | ST2 | ST2:LiQ | LiQ 1 nm |
| | 5 nm | 230 nm | 20 nm | (44%:44%:12%) | 10 nm | (50%:50%) | |
| | | | | 30 nm | | 30 nm | |
| I16 | HATCN | SpMA1 | SpMA2 | P251:CoH1:TEG1 | ST2 | ST2:LiQ | LiQ 1 nm |
| | 5 nm | 230 nm | 20 nm | (59%:29%:12%) | 10 nm | (50%:50%) | |
| | | | | 30 nm | | 30 nm | |
| I17 | HATCN | SpMA1 | SpMA2 | P332:CoH2:TEG2 | ST2 | ST2:LiQ | LiQ 1 nm |
| | 5 nm | 230 nm | 20 nm | (44%:44%:12%) | 10 nm | (50%:50%) | |
| | | | | 30 nm | | 30 nm | |
| I18 | HATCN | SpMA1 | SpMA2 | P393:CoH1:TEG1 | ST2 | ST2:LiQ | LiQ 1 nm |
| | 5 nm | 230 nm | 20 nm | (59%:29%:12%) | 10 nm | (50%:50%) | |
| | | | | 30 nm | | 30 nm | |

TABLE 2
Device data of the OLEDs

| | CIE x/y at | j0 | L1 | LT |
|---|---|---|---|---|
| Ex. | 10 mA/cm2 | (mA/cm²) | (%) | (h) |
| C1 | 0.34/0.62 | 40 | 80 | 100 |
| C2 | 0.34/0.62 | 40 | 80 | 105 |
| I1 | 0.34/0.62 | 40 | 80 | 260 |
| I2 | 0.36/0.61 | 40 | 80 | 130 |
| C3 | 0.34/0.62 | 40 | 80 | 180 |
| C4 | 0.35/0.62 | 40 | 80 | 140 |
| I3 | 0.34/0.62 | 40 | 80 | 400 |
| I4 | 0.35/0.62 | 40 | 80 | 240 |
| I5 | 0.35/0.63 | 40 | 80 | 520 |
| I6 | 0.35/0.62 | 40 | 80 | 220 |
| I7 | 0.35/0.63 | 40 | 80 | 480 |
| I8 | 0.35/0.63 | 40 | 80 | 500 |
| I9 | 0.35/0.62 | 40 | 80 | 210 |
| I10 | 0.35/0.62 | 40 | 80 | 220 |
| I11 | 0.35/0.63 | 40 | 80 | 420 |
| I12 | 0.35/0.62 | 40 | 80 | 205 |
| I13 | 0.35/0.63 | 40 | 80 | 450 |
| I13 | 0.35/0.62 | 40 | 80 | 210 |
| I15 | 0.35/0.63 | 40 | 80 | 495 |
| I16 | 0.35/0.62 | 40 | 80 | 230 |
| I17 | 0.35/0.63 | 40 | 80 | 490 |
| I18 | 0.35/0.62 | 40 | 80 | 210 |

TABLE 3
Structural formulae of the materials for the OLEDs 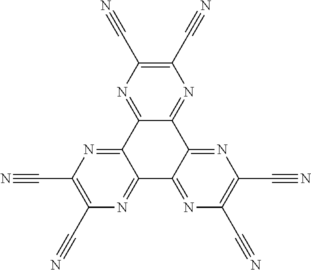 [SMILES: N#Cc1nc2c3nc(C#N)c(C#N)nc3c3nc(C#N)c(C#N)nc3c2nc1C#N]
HATCN 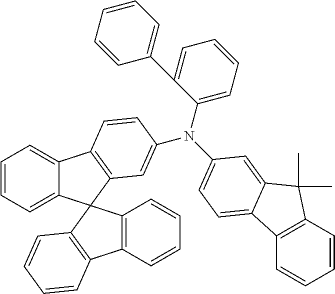 [SMILES: CC1(C)c2ccccc2-c2ccc(N(c3ccc4c(c3)C3(c5ccccc5-c5ccccc53)c3ccccc3-4)c3ccccc3-c3ccccc3)cc21]
SpMA1 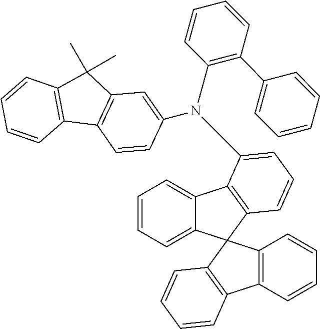 [SMILES: CC1(C)c2ccccc2-c2ccc(N(c3ccccc3-c3ccccc3)c3cccc4c3-c3ccccc3C43c4ccccc4-c4ccccc43)cc21]
SpMA2 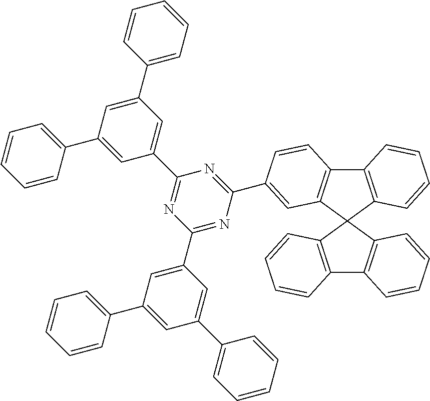 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]
ST2 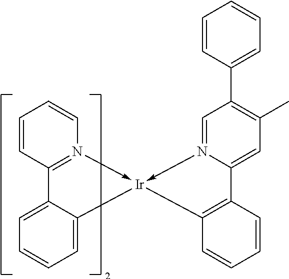
TEG1 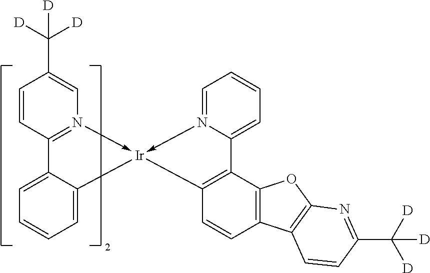
TEG2 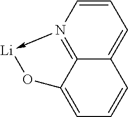
LiQ

1. Compound of formula (1) 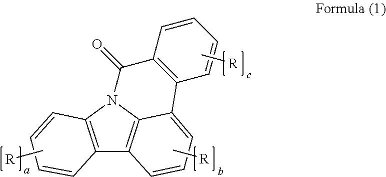
with the proviso that at least one R radical that represents a Y group is present, and where the symbols and indices used are as follows:
Y is the same or different at each instance and is a group of the formula (2), 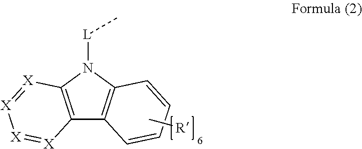
where the dotted bond indicates the linkage of this group in the formula (1);
X is the same or different at each instance and is CR′ or two adjacent X are a group of the formula (3), and the remaining X are the same or different at each instance and are CR′, 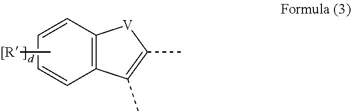
where the dotted bonds indicate the linkage of this group in the formula (2);
V is NR′, C(R′)₂, O or S;
L is an aromatic or heteroaromatic ring system which has 5 to 30 aromatic ring atoms and may be substituted by one or more R′ radicals;
R, R′ is the same or different at each instance and is H, D, F, Cl, Br, I, N(Ar′)₂, N(R¹)₂, OAr′, SAr′, CN, NO₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by Si(R¹)₂, C═O, NR¹, O, S or CONR¹, or an aromatic or heteroaromatic ring system which has 5 to 60 aromatic ring atoms, preferably 5 to 40 aromatic ring atoms, and may be substituted in each case by one or more R¹ radicals; at the same time, two R radicals together may also form an aliphatic, heteroaliphatic, aromatic or heteroaromatic ring system; in addition, two R′ radicals together may also form an aliphatic, heteroaliphatic, aromatic or heteroaromatic ring system; with the proviso that at least one R radical is a Y group;
Ar′ is the same or different at each instance and is an aromatic or heteroaromatic ring system which has 5 to 40 aromatic ring atoms and may be substituted by one or more R radicals;
R¹ is the same or different at each instance and is H, D, F, Cl, Br, I, N(R²)₂, CN, NO₂, OR², SR², Si(R²)₃, B(OR²)₂, COOR², C(═O)R², P(═O)(R²)₂, S(═O)R², S(═O)₂R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may each be substituted by one or more R² radicals, where one or more nonadjacent CH₂ groups may be replaced by Si(R²)₂, C═O, NR², O, S or CONR² and where one or more hydrogen atoms in the alkyl, alkenyl or alkynyl group may be replaced by D, F, Cl, Br, I or CN, or an aromatic or heteroaromatic ring system which has 5 to 40 aromatic ring atoms and may be substituted in each case by one or more R² radicals; at the same time, two or more R¹ radicals together may form an aliphatic, heteroaliphatic, aromatic or heteroaromatic ring system;
R² is the same or different at each instance and is H, D, F, CN or an aliphatic, aromatic or heteroaromatic organic radical, especially a hydrocarbyl radical, having 1 to 20 carbon atoms, in which one or more hydrogen atoms may also be replaced by F;
a is 0, 1, 2, 3 or 4;
b is the same or different at each instance and is 0, 1, 2 or 3;
c is 0, 1, 2, 3 or 4;
d is the same or different at each instance and is 0, 1, 2, 3 or 4;
with the proviso that a+b+c≥1;
with exclusion of the following compound from the invention:

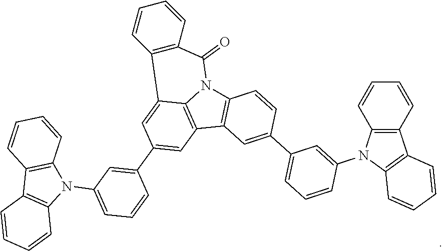 [SMILES: O=c1c2ccccc2c2cc(-c3cccc(-n4c5ccccc5c5ccccc54)c3)cc3c4cc(-c5cccc(-n6c7ccccc7c7ccccc76)c5)ccc4n1c23]

2. Compound according to claim 1, selected from the compounds of the formulae (4) to (6) 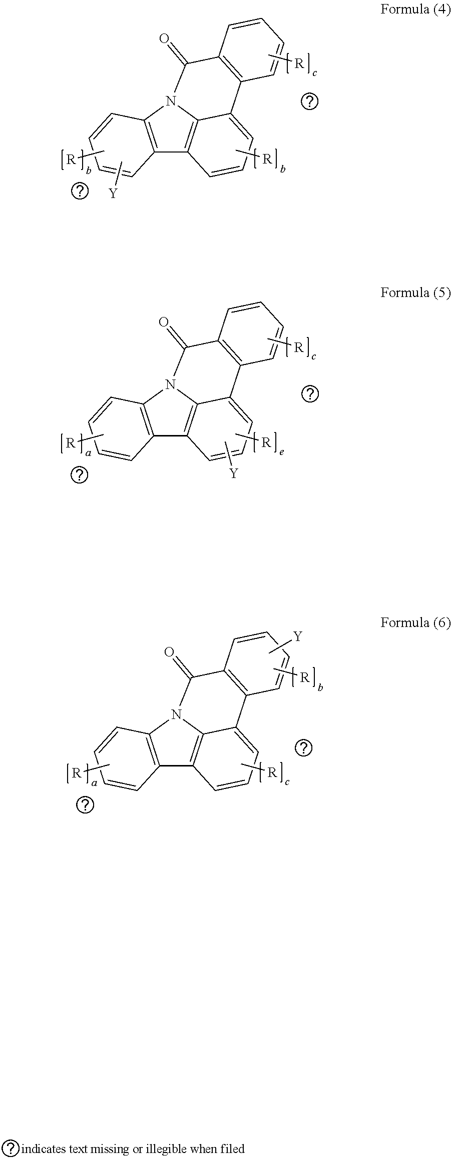
where the symbols and indices have the definitions given in claim 1 and in addition:
e is 0, 1 or 2.

3. Compound according to claim 1, selected from the compounds of the formulae (4a) to (6a) 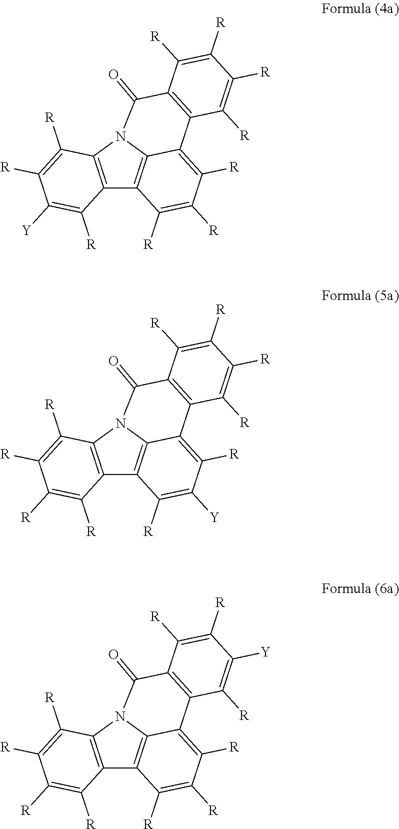
where the symbols have the definitions given in claim 1.

4. Compound according to claim 1, selected from the compounds of the formulae (4b) to (6b) 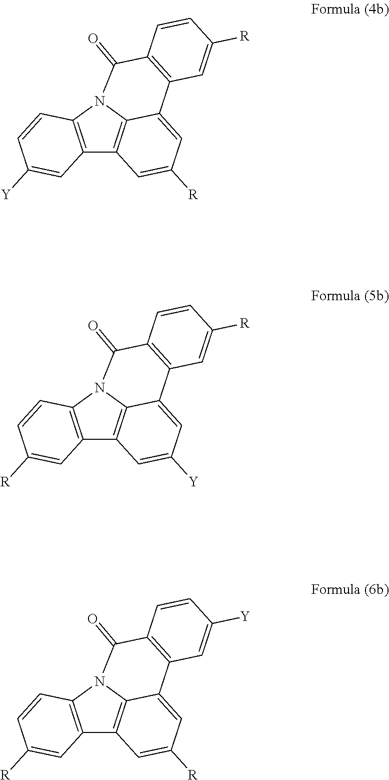
where the symbols have the definitions given in claim 1.

5. Compound according to claim 1, wherein exactly one or two R radicals are a Y group.

6. Compound according to claim 1, wherein L is the same or different at each instance and is an aromatic or heteroaromatic ring system which has 6 to 24 aromatic ring atoms and may be substituted by one or more R′ radicals, where L, when L is a heteroaromatic ring system, does not contain any electron-deficient heteroaryl groups.

7. Compound according to claim 1, wherein L is the same or different at each instance and is selected from the group consisting of benzene, biphenyl, terphenyl, quaterphenyl, fluorene, spirobifluorene, naphthalene, carbazole, dibenzofuran, dibenzothiophene, indenocarbazole, indolocarbazole, phenanthrene, triphenylene or a combination of two or three of these groups, where these groups may each be substituted by one or more R′ radicals.

8. Compound according to claim 1, wherein the Y group is the same or different at each instance and is selected from the groups of the following formulae (Y-1) to (Y-8): 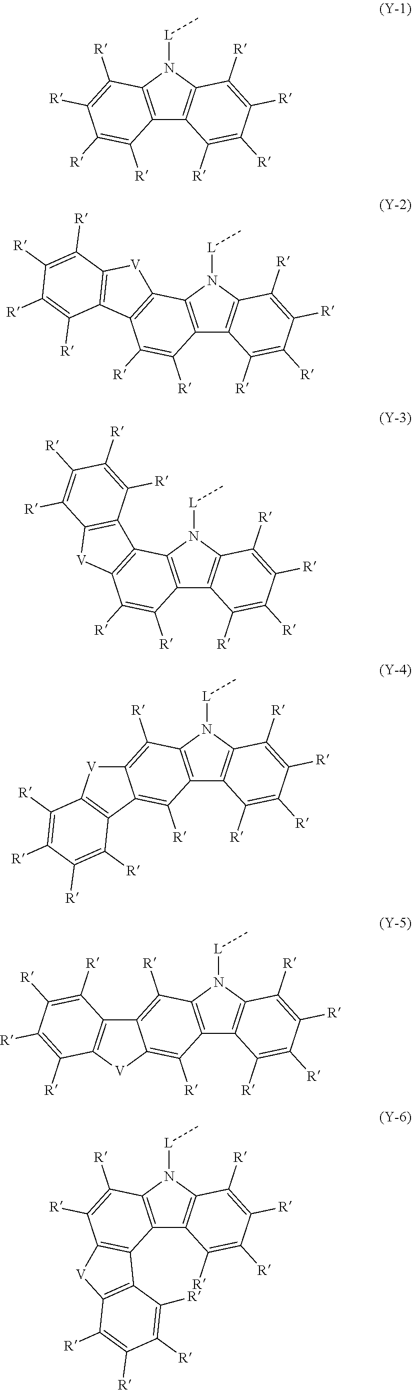 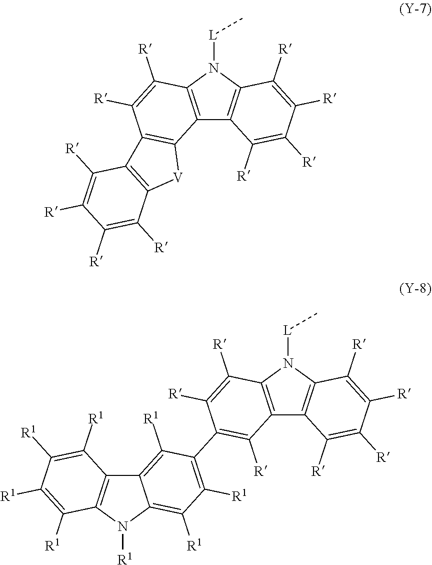
where the symbols have the definitions given in claim 1.

9. Compound according to claim 1, wherein the Y group is the same or different at each instance and is selected from the structures of the formulae (Y-1a) to (Y-8a) 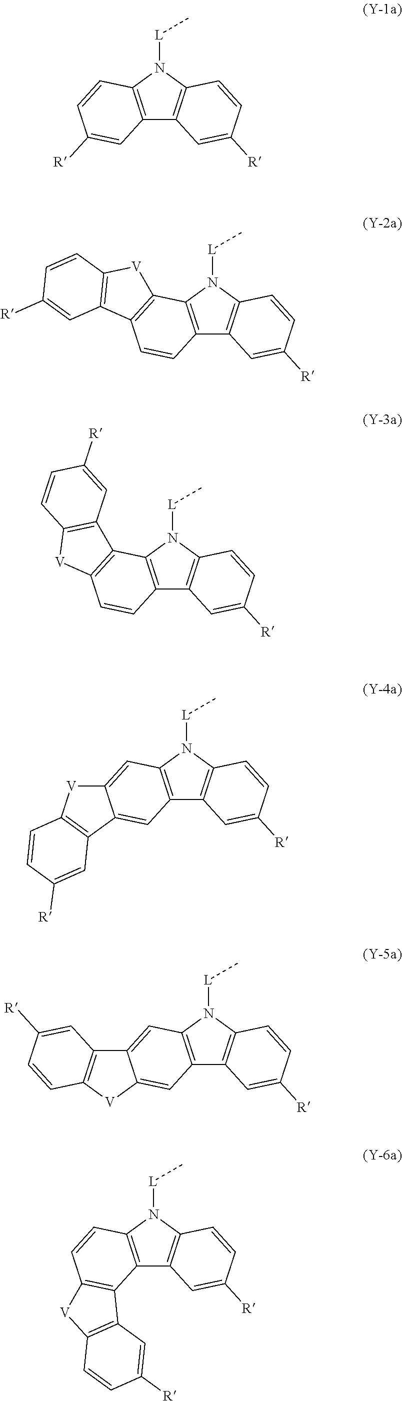 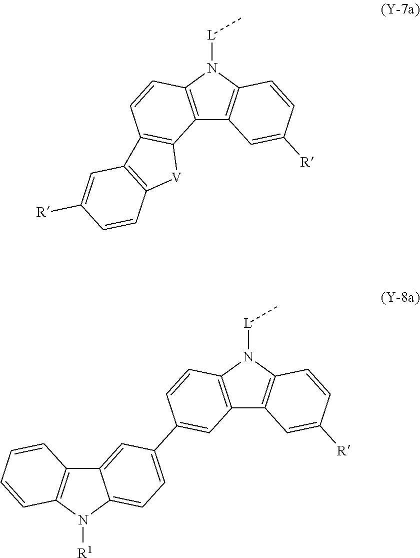
where the symbols have the definitions given in claim 1.

10. Compound according to claim 1, wherein R and R′ are the same or different at each instance and are selected from the group consisting of H, D, F, N(Ar′)₂, CN, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may each be substituted by one or more R¹ radicals, but is preferably unsubstituted, and where one or more nonadjacent CH₂ groups may be replaced by O, or an aromatic or heteroaromatic ring system which has 6 to 30 aromatic ring atoms and may be substituted in each case by one or more R¹ radicals; at the same time, two R radicals together may also form an aliphatic ring system; in addition, two R′ radicals together may also form an aliphatic or aromatic ring system.

11. Process for preparing a compound according to claim 1, characterized by the following synthesis steps:
a) synthesizing the lactam base skeleton that bears a reactive leaving group rather than the Y group; and
b) introducing the Y group by a coupling reaction.

12. Formulation comprising at least one compound according to claim 1 and at least one further compound and/or at least one solvent.

13. Use of a compound according to claim 1 in an electronic device.

14. Electronic device comprising at least one compound according to claim 1.

15. Electronic device according to claim 14 which is an organic electroluminescent device, wherein the compound according is used in an emitting layer as matrix material for phosphorescent emitters or for emitters that exhibit TADF and/or in an electron transport layer and/or in a hole blocker layer and/or in a hole transport layer and/or in an exciton blocker layer.
